(19) United States
(12) Patent Application Publication
(10) Pub. No.: US 2023/0226214 A1
(43) Pub. Date: Jul. 20, 2023

(54) BIFUNCTIONAL BRIDGING COMPOSITIONS FOR VIRAL TRANSDUCTION

(71) Applicant: Lycia Therapeutics, Inc., South San Francisco, CA (US)

(72) Inventors: Tigran Arvid AIVAZIAN, San Diego, CA (US); Richard James GLYNNE, La Jolla, CA (US); Dallas Clifford JONES, San Diego, CA (US); Stephanie Ann PINKERTON, San Marcos, CA (US); Darrin Anthony LINDHOUT, Mountain View, CA (US); Sarah M. MCWHIRTER, Albany, CA (US); Richard G. YAU, San Francisco, CA (US)

(21) Appl. No.: 18/012,840

(22) PCT Filed: Jun. 24, 2021
(86) PCT No.: PCT/US2021/039010

§ 371 Date: Dec. 23, 2022

Related U.S. Application Data
(60) Provisional application No. 63/043,769, filed on Jun. 24, 2020.
(60) Provisional application No. 63/043,772, filed on Jun. 24, 2020.
(60) Provisional application No. 63/135,547, filed on Jan. 8, 2021.
(60) Provisional application No. 63/214,169, filed on Jun. 23, 2021.

(51) Int. Cl. A61K 47/69; C07K 14/005; C12N 15/86; A61K 47/68; C07K 16/42; A61K 47/54; C07K 16/28; A61K 39/00

(52) U.S. Cl. CPC A61K 47/6901; C07K 14/005; C12N 15/86; A61K 47/6843; C07K 16/42; A61K 47/549; A61K 47/6889; A61K 47/6849; C07K 16/2863; A61K 2039/505; C12N 2750/14122; C07K 2317/622; C07K 2317/77; C12N 2750/14143; C07K 2317/76; C07K 2317/21; C12N 2750/14171; A61K 2039/545; C07K 2317/92; C07K 2317/24; C12N 2750/14145; C07K 2317/55

(57) ABSTRACT

This disclosure provides compositions and methods for delivering a viral composition to cells, e.g., for cell surface receptor-mediated uptake, and enhanced viral transduction. Viral transduction can be achieved via a bifunctional bridging composition that includes a moiety that binds to a cell surface receptor ligand and a linked bridging moiety that binds to a viral composition. Also provided are modified viral compositions comprising a bridging composition specifically bound via its bridging moiety to the viral composition. Modified viral compositions and methods for reducing levels or titers of neutralizing antibodies in a subject in need of viral therapy, e.g., gene therapy, are provided. In some embodiments, the modified viral composition includes empty viral particles that bind and internalize neutralizing autoantibodies. Modified viral compositions including empty viral particles can be administered prior to viral therapy. Also provided are pharmaceutical compositions and kits including a bifunctional bridging composition and/or modified viral compositions.

### 1. CROSS-REFERENCE TO RELATED APPLICATIONS

This application claims the benefit of U.S. Provisional Application No. 63/043,769, filed Jun. 24, 2020, which is hereby incorporated in its entirety by reference.

This application claims the benefit of U.S. Provisional Application No. 63/043,772, filed Jun. 24, 2020, which is hereby incorporated in its entirety by reference.

This application claims the benefit of U.S. Provisional Application No. 63/135,547, filed Jan. 8, 2021, which is hereby incorporated in its entirety by reference.

This application claims the benefit of U.S. Provisional Application No. 63/214,169, filed Jun. 23, 2021, which is hereby incorporated in its entirety by reference.

### 2. INTRODUCTION

### 2.1. Field of the Invention

Provided herein are bridging compositions, modified viral compositions and associated uses thereof. For example, provided herein are modified viral compositions comprising bridging composition presented herein specifically bound to viral compositions, for example, virus particles, virus capsids, or viral proteins, for example, capsid proteins or envelope proteins, and associated uses thereof.

### 2.2. Background of the Invention

A variety of viruses are used in the pharmaceutical and biotechnology industries. For example, adeno-associated virus (AAV) is a popular and versatile viral vector used in gene therapy. While viral vectors have broad potential and are widely implemented in therapeutic, manufacturing and research areas, there remains a need for viral vectors, particles and proteins, and methods of using the same, that exhibit or provide improved characteristics.

### 3. SUMMARY OF THE INVENTION

This disclosure provides compositions and methods for delivering a viral composition to for viral transduction. Viral transduction can be achieved via a bifunctional bridging composition that includes a moiety that binds to a cell surface receptor ligand and a linked bridging moiety that binds to a viral composition. Also provided are modified viral compositions comprising a bridging composition specifically bound via its bridging moiety to the viral composition. Upon binding of the cell surface receptor binding moiety to a target receptor present on a target cell, the bound modified viral composition is internalized into the cell. The viral composition can be a virus (virus particle), a virus capsid, virus envelope, or a viral protein (e.g., a viral capsid protein or viral envelope protein). In certain embodiments, the modified viral composition comprises a virus particle that comprises a polynucleotide that optionally comprises a transgene.

10008 The inventors demonstrated that exemplary bridging compositions including, e.g., an anti-AAV antibody and a M6PR binding moiety, can bridge to viral particles (e.g., AAV particles) and provide for cell surface receptor-mediated uptake, and enhanced viral transduction relative to unmodified viral particles alone. See e.g., FIGS. 25 and 29.

Aspects of this disclosure include modified viral compositions and methods for reducing levels of neutralizing antibodies in a subject in need of viral therapy, e.g., gene therapy. In some embodiments, the modified viral composition includes empty viral particles that can bind to and internalize autoantibodies that can neutralize a target viral particle, in some embodiments, the method includes administering the modified viral composition including empty viral particles prior to the onset of the viral therapy. The subject can be a human who has previously undergone viral therapy.

Also provided are pharmaceutical compositions and kits including a bifunctional bridging composition and/or modified viral compositions.

### 4. BRIEF DESCRIPTION OF THE SEVERAL VIEWS OF THE DRAWINGS

These and other features, aspects, and advantages of the present invention will become better understood with regard to the following description, and accompanying drawings.

FIG. 1 is a schematic of showing the binding of an exemplary viral particle (e.g., AAV) to an exemplary bridging composition (e.g., anti-AAV antibody-ligand conjugate), followed by cell surface receptor mediated internalization of the viral particle into the target cell.

FIG. 2 shows native mass spectrometry (MS) analysis of deglycosylated matuzumab and matuzumab-(Compound A) conjugate.

FIG. 3 shows native MS analysis of deglycosylated matuzumab and matuzumab-(Compound 1-7) conjugate.

FIG. 4 shows native MS analysis of deglycosylated atezolizumab and atezolizumab-(Compound A) conjugate.

FIG. 5 shows native MS analysis of deglycosylated cetuximab and cetuximab-(Compound A) conjugate.

FIG. 6 shows native MS analysis of deglycosylated cetuximab and cetuximab-(Compound I-7) conjugate.

FIGS. 7N-7B shows native MS analysis of deglycosylated anti-PD-L1 antibody (29E·2A3) and anti-PD-L1 antibody (29E·2A3)-(Compound A) conjugate.

FIG. 8 shows native MS analysis of deglycosylated IgG2a-UNLB and gG2a-UNLB-(Compound I-7) conjugate.

FIGS. 9A and 98 show western blot analyses of AAV8 particle-ligand conjugates indicating the presence of AAV proteins at the expected molecular weights (see FIG. 9B) and specific binding of an anti-MCP antibody to these conjugates (see FIG. 9A), thereby confirming the successful conjugation of Compound I-7 to the AAV8 particle. FIG. 9A: Western blot with anti-IMP antibody. FIG. 9B: Western Blot analysis with anti-AAV antibody. Lane 1: Mtz-UNLB. Lane 2: Mtz-Compound I-7 DAR 8. Lane 3: AAV8 Capsid. Lane 4: AAV8 Capsid+100,000× Compound I-7. Lane 5: AAV8 Capsid+50,000× Compound I-7, Lane 6: AAV8 Capsid+25,000× Compound I-7. Lane 7: AAV8 Capsid+10,000× Compound I-7. Mtz-UNLB refers to unlabeled matuzumab. Mtz-Compound I-7 DAR8 refers to matuzumab conjugated to Compound I-7 (DAR8).

FIGS. 10A and 10B illustrate that an AAV8 capsid conjugate containing a green fluorescence protein (GFP) transgene ((AAV8-CMV-GFP)-Compound I-7 conjugate) exhibits greater transduction efficiency than AAV8-CMV-GFP control alone. Data in FIG. 10A shows the transduction efficiency measured in human 2V6·11 cells, which express MPR on their cell surface, as a percentage of GFP positive cells, whereas FIG. 10B shows mean fluorescence intensity (MFI) of the GFP positive 2V6·11 cells.

FIG. 11 shows that an AAV8 capsid conjugate with Compound I-7 that includes a luciferase transgene exhibits greater transduction efficiency than unlabeled AAV8-luciferase in human 2V6·11 cells as measured by luciferase activity. RLU: relative light units, MOI: Multiplicity of Infection.

FIGS. 12A and 12B. AAV8 Capsid conjugates shows transduction efficiency in a transduction-resistant human Jurkat cell line as percentage of GFP-positive cells (FIG. 12A) or as Mean Fluorescence Intensity (MFI, FIG. 12B). “10 k” and “25 k” indicate the molar ratio of Compound I-7 to AAV8 (10,000:1 and 25,000:1, respectively) employed in the conjugation reaction.

FIGS. 13A-13D. ADK8 NAb does not reduce transduction efficiency of AAV8 capsid conjugate in human 2V6·11 cells (FIGS. 13A and 138) nor Jurkat cells (FIGS. 13C and 13D). GFP: Green Fluorescence Protein. MFI: Mean Fluorescence Intensity. Data shown as percentage of GFP-positive cells (FIGS. 13A and 13C) or MFI (FIGS. 13B and 13D). “10 k” and “25 k” indicate molar the ratio of Compound I-7 to AAV8 (10,000:1 and 25,000:1, respectively) employed in the conjugation reaction.

FIG. 14 shows a graph illustrating that AAV$ particle conjugate with ligand Compound I-7 retains its ability to bind to neutralizing antibody, ADK8. ADK8 neutralizing Ab (Nab) binding to GFP-AAV8 alone and to the AAV8 particle conjugate were measured using an ELISA kit (Progen) according to manufacturer's instructions. The data shown in FIG. 14 indicates that the AAV8 particle-conjugate retains an ability to bind to ADK8 similar to that of AAV8 alone.

FIG. 15. Increased transduction efficiency of AAV8 particle conjugate is dependent on the ell surface expression of M6PR. Utilizing a human K562 cell line that either expresses M6PR on the cell surface (M6PR^{POS}) and a companion K562 cell line where CPR has been deleted (M6PR^{NEG}) in transduction studies demonstrates the increased transduction efficiency of the AAV8 particle conjugated to Compound I-7 only observed in the M6PR^{POS} K562 cell line.

FIGS. 16A-16D. Increased transduction efficiency of AAV8 particle conjugate is not observed when conjugated to inactive enantiomer of Compound I-7. FIGS. 16A and 16C show transduction efficiency of 2V6·11 cells with AAV8 conjugated to the active enantiomer of Compound I-7 and FIGS. 16B and 16D show transduction efficiency of human 2V6·11 cells with AAV8 conjugated to the inactive enantiomer of Compound I-7. FIGS. 16A and 16B show the transduction efficiency as a percentage of GFP positive cells, whereas FIGS. 16C and 16D show mean fluorescence intensity (MM.

FIGS. 17A-17F. Binding affinities for M6PR of matuzumab conjugated to unlabeled control (FIG. 17A), Compound I-7 (FIG. 17B), Compound I-8 (FIG. 17C), Compound I-9 (FIG. 17D), compound 1-11 (FIG. 17E) and Compound I-12 (FIG. 17F) to M6PR. Binding to M6PR was determined by ELISA. Compound I-7 (dar8) and Compound I-11 (dar4) showed the highest and lowest binding affinity, respectively. RFU: Relative fluorescence units.

FIGS. 18A-18C. Serum pharmacokinetic (PK) analysis shows that individual rIgG1 antibody conjugates with compounds having relatively weaker binding affinity to M6PR can exhibit longer half-life, and therefore can be useful for tuning desired PK properties. Intracellular levels of anti-IgG2a conjugate Compound I-7 (dar8) and (dar4) (FIG. 18A), anti-IgG2a conjugate Compound I-10 and anti-IgG2a conjugate Compound I-11 (FIG. 1813), and anti-IgG2a conjugate Compound I-9 and anti-IgG2a conjugate Compound I-12 (FIG. 18C) in mouse serum were measured at 0.5, 1, 2, 6, and 24 hours using ELISA.

FIG. 19. Intracellular uptake of anti-IgG2a conjugates over time in human Jurkat cells. Conjugates were detected using Alexa488-conjugated antibodies, and intracellular levels of fluorescence were determined by FACS after 1 h and 24 h.

FIG. 20. Intracellular uptake of anti-IgG2a conjugates into human Jurkat cells at 10 nM after 24 hours as a percentage of the update of anti-IgG2a conjugate Compound I-7 dar8.

FIGS. 21A and 21B. AAV8 particles conjugated to Compound I-7 (ITX-16590) and Compound I-124 (ITX-22701) exhibit greater transduction efficiency than unconjugated AAV8. Data in FIG. 21A shows the transduction efficiency as a percentage of GFP positive cells. FIG. 21B shows mean fluorescence intensity (MFI).

FIGS. 22A and 22B. Transgene expression of conjugated and unconjugated AAV8 particles. Data in FIG. 22A shows luciferase expression (RLU) of human 2v6·11 cells transduced with conjugated or unconjugated AAV8 particles in a range of dilutions of pooled human serum. FIG. 22B is an enlargement of FIG. 22A for the 1 to 100 dilution range.

FIGS. 23A and 23B. Bioluminescence imaging of mice dosed with unconjugated AAV8-luciferase (FIG. 23A) or AAV8-luciferase conjugated to GalNAc (FIG. 23B).

FIG. 24: Bioluminescence imaging of mice dosed with unconjugated AAV8-luciferase (top), AAV8-luciferase conjugated to GalNAc (bottom), and AAV8-luciferase conjugated to GalNAc enantiomer (right) at doses of 1×10¹¹, 3×10¹⁰, or 1×10¹⁰ vg per mouse.

FIG. 25. Anti-AAV antibody compound conjugate can bridge AAV particle and cell surface binding moiety and increase transduction efficiency. FIG. 25 shows transduction efficiency of AAV8-luciferase in the presence of Compound I-7 conjugated and unconjugated anti-AAV8 antibody (ADK8) in human 2V6·11 cells.

FIG. 26 shows a schematic of an exemplary bifunctional compound that includes an antibody linked to two IGF-2 polypeptides, Site specific covalent linkages to the antibody can be achieved via conjugation of a chemoselective bivalent linker, such as 6-malemidocaproic acid sulfo-NHS. The linker can be installed on an IGF-2 polypeptide via e.g., NHS chemistry and coupling of the linker to an N-terminal amino group or sidechain lysine group of the IGF-2 polypeptide. The maleimide chemoselective ligation group of the linker shown in FIG. 1 is reactive with cysteine residues on the antibody, such as a cysteine residue engineered into the antibody at desirable site-specific locations, e.g., L443C. It is understood that a variety of cell surface receptor ligand-linker compounds described herein can also be configured as shown in FIG. 26 via site specific conjugations.

FIG. 27 shows a schematic of an exemplary bifunctional compound that includes an antibody fused to four IGF-2 polypeptides at the C-terminals of the immunoglobulin heavy and light chains of the antibody. The bifunctional compound can be a fusion protein where IGF-2 polypeptides are incorporated into the architecture of an antibody at a variety of suitable sites.

FIGS. 28A-28C shows cell uptake of exemplary bridging compositions with conjugates of antibody omalizumab to glycan ligands for M6PR (mannose-6-phosphate ligand (M6P) or mannose-6-phosphonate analog (M6Pn, e.g., Compound I-7) or IGF-2 polypeptide (designed “IGF2” in the graph legend) versus unconjugated omalizumab (UNLB) in three different cell types, FIG. 28A shows uptake in human Jurkat cells. FIG. 28B shows uptake in mouse C2C12 cells. FIG. 28C shows uptake in mouse fibroblasts. The cellular uptake is compared to two different omalizumab conjugates having glycan ligands for M6PR (i.e., a linked mannose-6-phosphate glycan conjugate designated “M6P”, or to unconjugated omalizumab (i.e., “UNLB”). Each of the omalizumab compositions tested was fluorescently labelled with an Alexa 488 fluorophore reagent dye to provide for assessment of cellular uptake via mean fluorescent intensity (MFI) of cells using flow cytometry.

FIG. 29 shows the results of a cellular uptake assay illustrating that exemplary bifunctional compound IGF-2-omalizumab (“IGF2”) is internalized into wild type human K562 cells having M6PR but not into K562 M6PR-knockout (KO) cells, Similar results were observed for omalizumab conjugates with the glycan ligands for M6PR (“M6P” or “1\46Pn” (Compound I-7)). “UNLB” is the control unconjugated omalizumab. Cellular uptake was assessed using flow cytometry to determine mean fluorescent intensity (MN) of cells.

FIG. 30. Increasing concentration of Compound I-7-αIgG2a conjugate can clear ADK8 antibody and increase in vitro AAV8-luciferase transduction in human Jurkat cells (25 k/well for 72 hours). aIgG2a alone does not promote clearing of ADK8.

FIG. 31 illustrates that increased transduction efficiency was observed for AAV9 conjugated to Compound I-7 compared to unconjugated AAV9 (“AAV9-Unlabeled”) at all but the highest molar ratio of Compound I-7 to AAV9 tested. “10K,” “50K,” “100K,” and “200K” indicate the molar ratio of Compound I-7 to AAV9. The molar ratio of Compound I-7 to AAV9 of 100,000:1 (“100K”) shows the best transduction.

FIGS. 32A-32D show improved transduction efficiency of the AAV8 conjugates compared to unconjugated AAV8 in all four human primary cell lines tested. Transduction with AAV8 Luciferase conjugated to Compound I-7 (“AAV8 Luc-Cmpd I-7”) resulted in increased transgene expression compared to unconjugated AAV8 Luciferase (“AAV8 Luc”) in primary human fibroblasts (FIG. 32A), primacy human endothelial cells (FIG. 32B), primary human hepatocytes (FIG. 32C), and primary human skeletal muscle cells (FIG. 32D). Luciferase expression was particularly high in fibroblasts (FIG. 32A) and hepatocytes (FIG. 32C) transduced with AAV8 particle conjugates, demonstrating that conjugation improves transduction efficiency and transgene expression especially well in these human cell types.

### 5. DETAILED DESCRIPTION OF THE INVENTION

### 5.1. Compositions for Viral Transduction

As summarized above, this disclosure provides compositions and methods for delivering a viral composition to cells, e.g., for viral transduction. Viral transduction can be achieved using a bifunctional bridging composition that includes a moiety that binds to a cell surface receptor ligand. The bifunctional bridging composition can include a bridging moiety attached to (e.g., fused or conjugated to, either directly or indirectly, e.g., via linker) the cell surface binding moiety, wherein the bridging moiety binds to a viral composition, for example, a virus s particle), a virus capsid, virus envelope, or a viral protein (e.g., a viral capsid protein or viral envelope protein), In curtain aspects, provided herein are modified viral compositions comprising bridging compositions specifically bound to viral compositions. In certain embodiments, a modified viral composition comprises a virus particle that comprises a polynucleotide that optionally comprises a transgene.

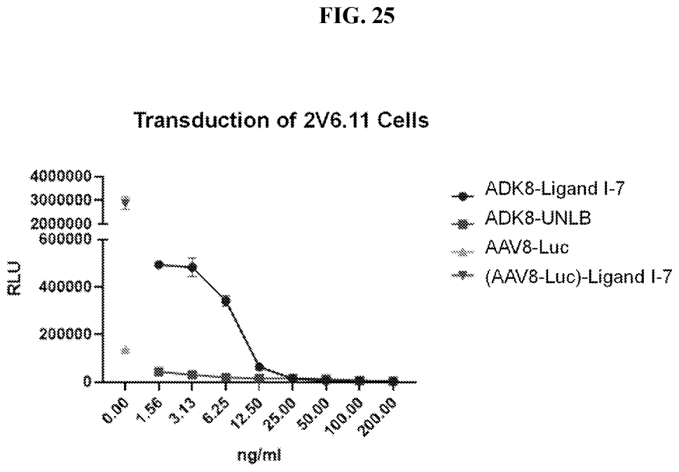

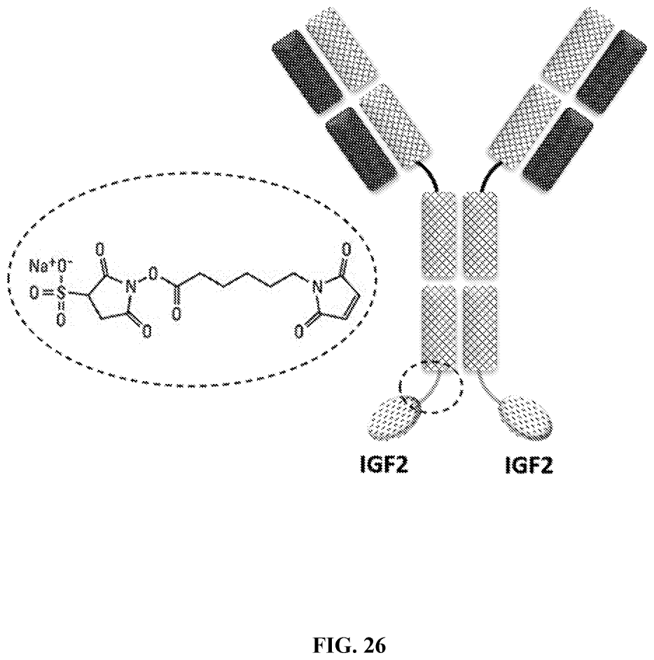

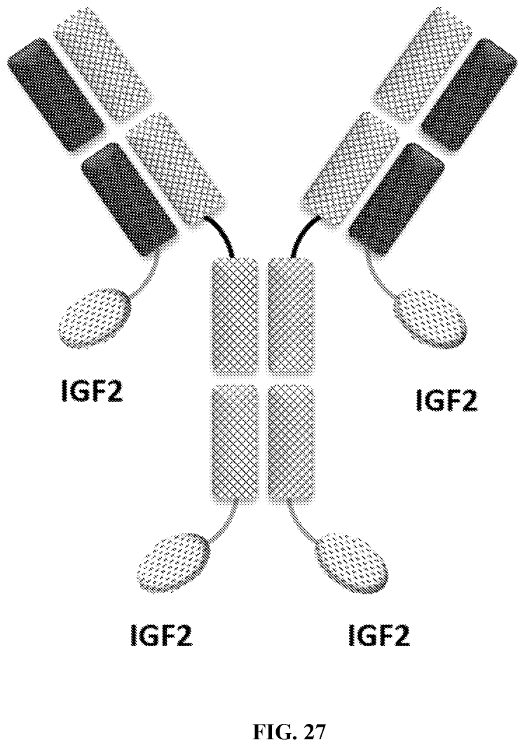

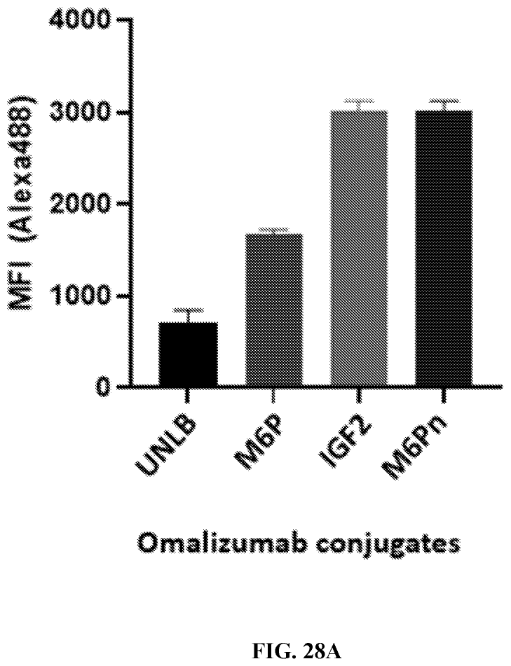

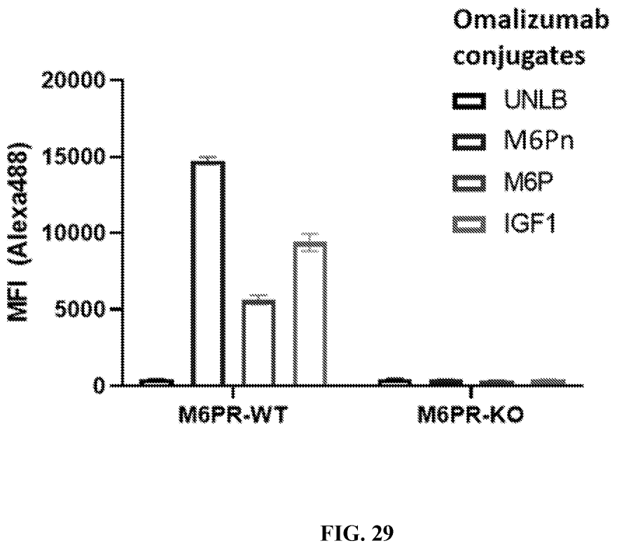

Accordingly, aspects of this disclosure include a modified viral composition that finds use in the methods of viral transduction. The modified viral composition can include a bridging composition and a viral composition to which the bridging moiety of the bridging composition is capable of binding non-covalently, e.g., in a complex. The inventors have demonstrated that upon binding of the cell surface receptor binding moiety (e.g., ligand) to a target receptor present on a target cell, the modified viral composition is internalized into the cell. The modified viral compositions can provide for a higher transduction efficiency as compared to an unmodified virus composition, e.g., a viral particle not bound to a bifunctional bridging composition. See e.g., FIGS. 25 and 29.

In certain embodiments, upon binding to a cell surface receptor present on a cell, a modified viral composition internalizes into the cell. In certain embodiments, the cell surface receptor is an endocytic receptor that mediates endocytosis of the modified viral composition into the endocytic pathway of the cell. In particular embodiments, the cell surface receptor is a M6P receptor (M6PR). Iii other embodiments, the cell surface receptor is a folate receptor, e.g., a folate receptor 1 (FRα), or 2 (FRβ) receptor, or an asialoglycoprotein receptor.

In certain embodiments, the cell surface receptor is an endocytic receptor that mediates endocytosis of molecules into a cell, where the molecules are directed to the cell's endocytic pathway. The modified viral composition comprises a cell surface receptor binding moiety that binds to (e.g., is a ligand of) an endocytic receptor. Without wishing to be bound by theory or mechanism, upon binding of a modified viral composition comprising an endocytic receptor ligand to a cell surface endocytic receptor, the modified viral composition may be internalized into the cell, where it becomes contained within endosomes, and may then be translocated or directed via the endocytic pathway to other locations or vesicles (e.g., to the trans-golgi network, to lysosomes or recycled back to the cell surface).

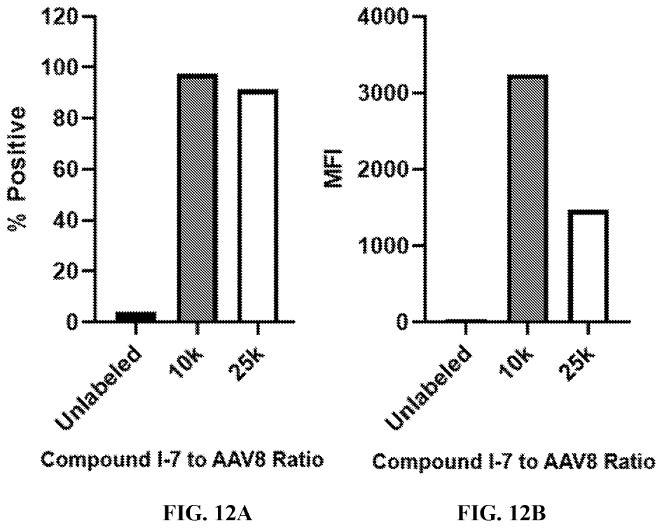

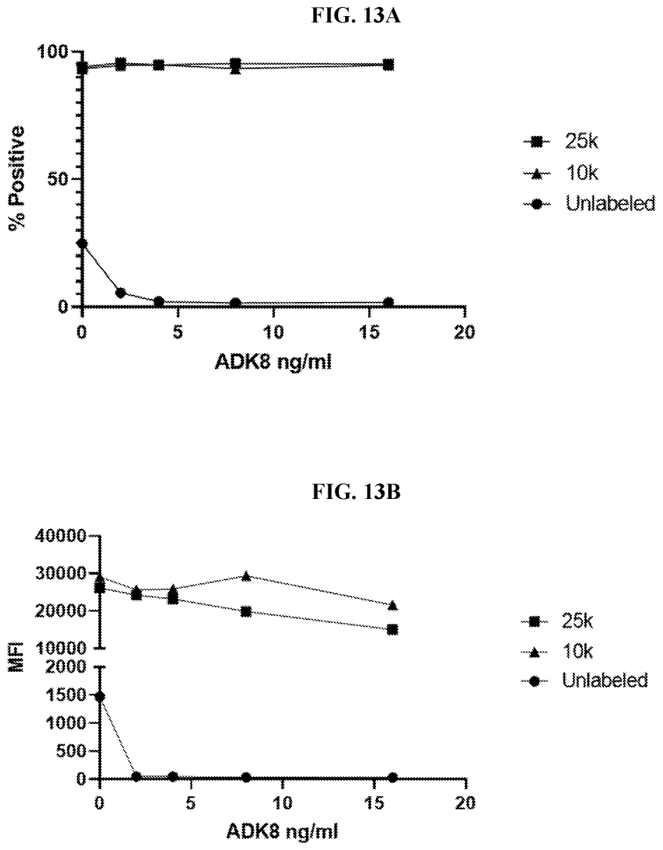

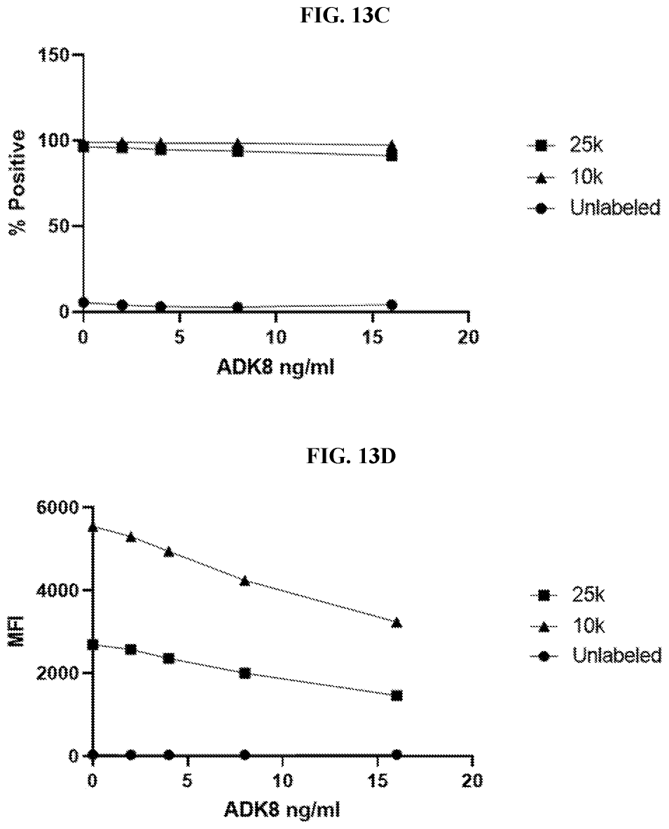

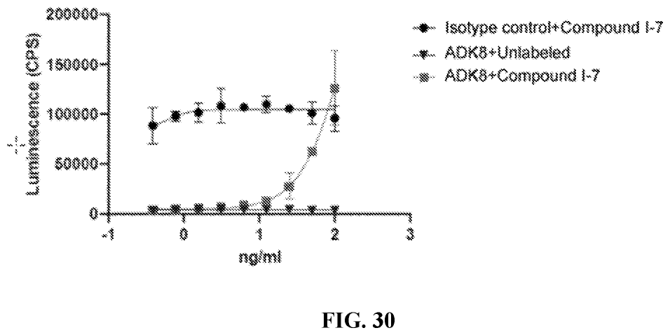

The inventors have demonstrated that neutralizing antibody (NAb) for exemplary modified viral compositions (e.g., compound I-7 modified AAV particles as compared to an unmodified viral particles) does not reduce transgene transduction efficiency of a modified viral composition. In addition, the inventors show that level of a neutralizing Ab can be cleared via cell surface receptor mediated endocytosis to provide for increased viral transduction of an unmodified viral composition. See e.g., FIGS. 13 and 30.

Accordingly, aspects of this disclosure include methods of using modified viral compositions to reduce levels of neutralizing antibodies that can adversely affect the transduction of viral compositions, e.g., for gene therapy applications. In some embodiments, a modified viral composition (e.g., as described herein) including empty viral particles (i.e., no transgene cargo) is administered as a decoy to bind and internalize autoantibodies that neutralize the target viral particle, thereby reducing levels of the neutralizing autoantibody prior to administration of a therapeutic viral composition. Thus, aspects of this disclosure include a modified viral composition that has empty decoy viral particles of a target viral particle serotype.

### 5.2. Bifunctional Bridging Compositions

Aspects of this disclosure include a bifunctional bridging composition that includes a bridging moiety and a cell surface receptor binding moiety.

The bridging moiety and the cell surface receptor binding moiety can be covalently attached to each other directly, or indirectly via an intervening structure, for example a linker. For example, the bridging moiety can be directly conjugated to the cell surface binding moiety, or may be conjugated via a bivalent linker. In another example, the bifunctional bridging composition may comprise a proteinaceous bridging moiety fused directly to a proteinaceous cell surface receptor binding moiety, or fused via an intervening structure, for example spacer polypeptide sequence(s).

In some embodiments, the bifunctional bridging composition is of formula (I):

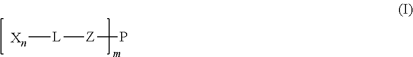

or a pharmaceutically acceptable salt thereof, wherein:

X is a moiety that binds to a cell surface receptor;

L is an optional linker;

n is 1 to 500 (e.g., 1 to 20, 1 to 10, or 1 to 5);

m is 1 to 80;

Z is a residual moiety resulting from the covalent linkage of Y to P, where Y is a moiety that covalently bonds to a bridging moiety (e.g., a chemoselective ligation group); and

P is a bridging moiety (e.g., a polypeptide such as an antibody or antibody fragment).

In some embodiments of formula (I), the bifunctional bridging composition is of formula (Ib):

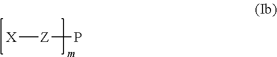

wherein in is an integer from 1 to 80, and Z is a residual moiety resulting from the covalent linkage of Y to P (e.g., a polypeptide such as an antibody or antibody fragment), e.g., via a chemoselective ligation group (e.g., as described herein).

In some embodiments of formula (I)-(Ib), m is an integer of 1 to 20, such as in is 1 to 10, e.g., 2 to 10 or 2 to 6.

In some embodiments of formula (I), n is 1 to 5, such as n is 1 to 3, e.g., n is 1 or 2.

5.2.1. Cell-Surface Receptor Binding Moieties

The bifunctional bridging compositions include a binding moiety for a cell surface receptor that facilitates internalization and delivery of the bifunctional bridging compositions and a bound viral composition to a target cell. By selection of a suitable binding moiety, a variety of cell surface receptors can be targeted by the bifunctional bridging composition of this disclosure.

In some embodiments, the bridging composition binds to an endocytic receptor s a ligand for the endocytic receptor).

Cell surface receptors of interest include, but are not limited to, mannose-6-phosphate receptors, asialoglycoprotein receptors, folate receptors, mannose receptor, low density lipoprotein receptor-related protein 1 (LRP1) receptor, low density lipoprotein receptor (LDLR), FcγRI receptor, transferrin receptor, macrophage scavenger receptor, and G-Protein coupled receptor.

In some embodiments, the cell surface receptor binding moiety is a folate receptor binder, mannose receptor binder, mannose-6-phosphate (M6P) receptor binder, low density lipoprotein receptor-related protein 1 (LRP1) receptor binder, low density lipoprotein receptor (LDLR) binder, FcγRI receptor binder, transferrin receptor binder, macrophage scavenger receptor binder, G-Protein coupled receptor binder, or asialoglycoprotein receptor (ASGPR) binder.

In some embodiments, the cell surface receptor binding moiety is a proteinacious molecule, for example, a moiety that comprises a polypeptide. In some embodiments, the cell surface receptor binding moiety is an antibody or antibody fragment. In some embodiments, the cell surface receptor binding moiety comprises a small molecule, for example, is a small molecule. In some embodiments, the cell surface receptor binding moiety is a glycoprotein. In some embodiments, the cell surface receptor binding moiety comprises a sugar moiety or glycan.

The bridging compositions of this disclosure can be prepared from compounds including a binding moiety (e.g., ligand) that specifically binds to a cell surface receptor via conjugation with a bridging moiety that binds to a viral composition. It is understood that any of the precursor compounds described herein can find use in preparing a bridging composition, and that embodiments of all such bridging composition products and modified viral compositions are meant to be included in this disclosure.

In one aspect, described herein are compounds including a cell surface receptor binding moiety that can be linked to a bridging moiety to prepare a bridging composition of formula (I), and are of formula (Ia):

Xₙ-L-Y (Ia)

or a salt thereof, wherein:

X is a moiety that hinds to a cell surface receptor;

n is 1 to 500;

L is an optional linker of defined length; and

Y comprises a moiety that covalently bonds to a bridging moiety.

The compounds of formula (Ia) can be adapted for use in preparing bridging compositions (e.g., as described herein). Such conjugates can be prepared by conjugation of a chemoselective ligation group of any one of the compounds described herein with a compatible reactive group of a component of a bridging moiety composition (e.g., as described herein). The compatible reactive group of the bridging moiety can be introduced by modification prior to conjugation, or can be a group present in the molecule.

The compounds and conjugates that can be adapted for use in the bridging compositions of this disclosure are described in greater detail below. A particular class of M6PR binding compounds is described. Also described is a particular class of ASGPR binding compounds. Also described is a particular class of folate receptor binding compounds. Linkers (L) and chemoselective ligation groups which find use in the compounds and conjugates are also described.

A variety of other cell surface receptor binding compounds can be adapted for use in the subject bridging compositions, including but not limited to, those described in WO2021/072246, WO2021/072269, US2018/0265534, and WO 2020/132100, the disclosures of which are herein incorporated by reference.

A compound or bridging composition comprising such X (e.g., as described herein), may bind to other receptors, for example, may bind with lower affinity as determined by, e.g., immunoassays or other assays known in the art. In a specific embodiment, X, or a compound as described herein comprising such X specifically binds to the cell surface receptor with an affinity that is at least 2 logs, 2.5 logs, 3 logs, 4 logs or greater than the affinity when X or the compound or the conjugate bind to another cell surface receptor. In a specific embodiment, X, e.g., M6P or an M6P analog or derivative, or a compound as described herein comprising X, specifically binds to a target cell surface receptor with an affinity (K_{d}) of 10 uM or less, such as 1 uM or less, 100 nM or less, or even 1 nM or less.

5.2.1.1 M6PR Binding Moieties

In some embodiments, the cell surface receptor targeted by the bifunctional bridging compositions of this disclosure is an internalizing mannose-6-phosphate receptor (M6PR).

The term “mannose-6-phosphate receptor” and “M6PR” refer to receptors of the family of mannose-6-phosphate receptors. M6PRs are transmembrane glycoprotein receptors that target enzymes to lysosomes in cells. MP6R endogenously transports proteins bearing N-glycans capped with mannose-6-phosphate (M6P) residues to lysosomes, and cycles between endosomes, the cell surface, and the Golgi complex. See, e.g., Ghosh et al., Nat. Rev. Mol. Cell Biol. 2003; 4: 202-213. The family of M6PRs includes the cation independent mannose-6-phosphate receptor (CI-M6PR). The CI-M6PR is also referred to as the insulin-like growth factor 2 receptor (IGF2R) and is encoded in humans by the IGF2R gene (sec NCBI Gene ID: 3482), The CI-M6PR binds insulin-like growth factor 2 (IGF-2) and mannose-6-phosphate (M6P)-tagged proteins.

In some embodiments, the surface M6PR is a human M6PR. In some embodiments, the M6PR is Homo sapiens insulin like growth factor 2 receptor (IGF2R) (see, e.g., NCBI Reference Sequence: NM_000876.3), also referred to as cation-independent mannose-6-phosphate receptor (CI-MPR).

In some embodiments of formula (Ia), X is a moiety that binds to a cell surface M6PR (e.g., M6PR ligand or binding moiety, e.g., as described herein).

5.2.1.1.1 M6P Small Molecule Ligands

The bridging compositions of the disclosure can include a ligand or binding moiety that specifically binds to a cell surface mannose-6-phosphate receptor (M6PR), The M6PR Ligand binding moieties can be linked to a variety of bridging moieties without impacting the specific binding to, and function of, the cell surface M6PR. The inventors have demonstrated that M6PR ligand binding moieties of this disclosure can utilize the functions of cell surface M6PRs in a biological system, e.g., for internalization of a modified viral composition.

In some embodiments, the cell surface receptor binding moiety comprises a sugar moiety, for example, mannose-6-phosphate (M6P) or a variant thereof. Mannose-6-phosphate (M6P) is a naturally occurring ligand for M6PR receptors.

The M6PR binding compounds of formula (Ia) can include a moiety (X) that specifically binds to the cell surface receptor M6PR. For example, a mannose-6-phosphate (M6P) or an M6P analog or derivative (e.g., as described herein), that specifically binds to a cell surface M6PR. The M6PR binding compounds can be monovalent or multivalent (e.g., bivalent or trivalent or of higher valency), where a monovalent compound includes a single M6PR ligand moiety, and a monovalent compound includes two or more such moieties.

In some embodiments, the M6PR binding moiety X includes a mannose sugar ring, or analog thereof, with a hydrophilic head group that is linked via a linking moiety to the 5-position of the ring. The linking moiety can be of 1-6 atoms in length, such as 1-5, 1-4 or 1-3 atoms in length. The hydrophilic head group can be any convenient group that is charged or readily capable of hydrogen bonding or electrostatic interactions under aqueous or physiological conditions. The hydrophilic head group can be a structural or functional mimic of the 6-phosphate group of M6P that has desirable stability. The hydrophilic head group can have a MW of less than 200, such as less than 150 or less than 100. In some embodiments, the hydrophilic head group is a phosphonate. In some embodiments, the hydrophilic head group is a thiophosphonate. In some embodiments, the hydrophilic head group is a phosphate, thiophosphate or dithiophosphate.

In some embodiments, the mannose sugar ring of X is linked to an optionally substituted amyl or heteroaryl group that together provide a moiety having a desirable binding affinity and activity at the M6P receptor of interest. Multiple M6PR binding moieties X can be linked together to provide multivalent binding to the M6PR. The M6PR binding moiety or moieties X can be further linked to any convenient moiety or molecule of interest (e.g., as described herein).

The M6PR binding moiety (X) of the compounds of this disclosure can include a mannose ring or analog thereof described by the following structure:

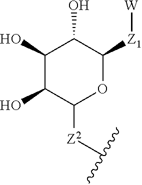

where:

W is a hydrophilic head group;

Z¹ is selected from optionally substituted (C₁-C₃)alkylene and optionally substituted ethenylene;

Z² is selected from O, S, NR²¹ and C(R²²)₂, wherein each R²¹ is independently selected from H, and optionally substituted (C₁-C₆)alkyl, and each R²² is independently selected from halogen (e.g., F) and optionally substituted (C₁-C₆)alkyl.

The mannose ring or analog thereof of the M6PR binding moiety can be incorporated into the bridging compositions of this disclosure by attachment to the Z² group via a linking moiety. It is understood that in the compounds of formula (Ia), the group or linking moiety attached to Z² can, in some cases, be considered to be part of the M6PR binding moiety (X) and provide for desirable binding to the M6PR. In certain other cases, the group or linking moiety attached to Z² can be considered part of the linker L of formula (Ia).

In one aspect, provided herein are cell surface mannose-6-phosphate receptor (M6PR) binding bifunctional compounds of formula (XI):

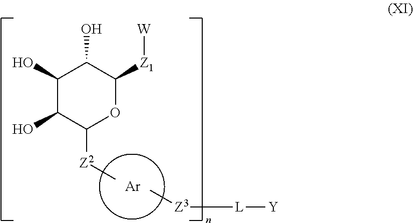

or a salt thereof, wherein:

each W is independently a hydrophilic head group;

each Z¹ is independently selected from optionally substituted (C₁-C₃)alkylene and optionally substituted ethenylene;

each Z² is independently selected from O, S, NR²¹ and C(R²²)₂, wherein each R²¹ is independently selected from H, and optionally substituted (C₁-C₆)alkyl, and each R²¹ is independently selected from H, halogen (e.g., F) and optionally substituted (C₁-C₆)alkyl;

each Ar is independently an optionally substituted aryl or heteroaryl group or linking moiety;

each Z³ is independently a linking moiety;

n is 1 to 500;

L is a linker; and

Y is a bridging moiety.

The Ar group linking moiety of formula (XI) can be a monocyclic aryl or monocyclic heteroaryl group. In some embodiments of formula (XI), Ar is a 5-membered monocyclic heteroaryl group. In some embodiments of formula (XI), Ar is a 6-membered monocyclic aryl or heteroaryl group. The Ar group linking moiety of formula (XI) can be a multicyclic aryl or multicyclic heteroaryl group, such as a bicyclic aryl or bicyclic heteroaryl group. In some embodiments of formula (XI), Ar is a fused bicyclic group. In some embodiments of formula (XI), Ar is a bicyclic group comprising two aryl and/or heteroaryl monocyclic rings connected via a covalent bond. In some embodiments of formula (XI), Ar is a bicyclic an or bicyclic heteroaryl group having two 6-membered rings. In some embodiments of formula (XI), Ar is a bicyclic aryl or bicyclic heteroaryl group having one 6-membered ring that is connected via a covalent bond or fused to a 5-membered ring.

In some embodiments of formula (XI), each Ar is independently selected from optionally substituted phenyl, optionally substituted pyridyl, optionally substituted biphenyl, optionally substituted naphthalene, optionally substituted quinoline, optionally substituted triazole and optionally substituted phenylene-triazole. In some embodiments of formula (XI), Ar is substituted with at least one OH substituent. In some embodiments of formula (XI), Ar is substituted with 1, 2, or more OH groups. In some embodiments of formula (XI), Ar is substituted with at least one optionally substituted (C₁-C₆)alkyl.

In some embodiments of formula (XI), Ar is optionally substituted 1,4-phenylene, optionally substituted 1,3-phenylene, or optionally substituted 2,5-pyridylene.

In some embodiments of formula (XI), the compound is of formula (XIIa) or (XIIb):

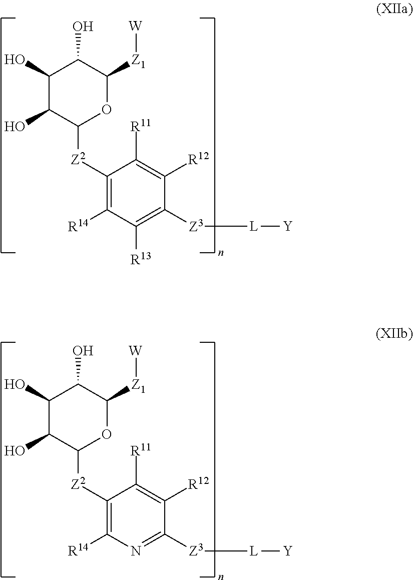

or a salt thereof, wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

In some embodiments of formula (XIIa)-(XIIb), R¹¹ to R¹⁴ are each H. In some embodiments of formula (XIIa)-(XIIb), at least one of R¹¹ to R¹⁴ is OH, such as 1, 2, or more of R¹¹ to R¹⁴ is OH.

In some embodiments of formula (XIIa)-(XIIb), Z³ is selected from a covalent bond, —O—, —NR²—, —NR²³CO—, —CONR²³—, —NR²³CO₂—, —OCONR²³, —NR²³C(═X¹)NR²³—, —CR²⁴═N—, —CR²⁴═N—X², —NR²³SO₂—, and —SO₂NR²³—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (XI)-(XIIb), Z³ is a covalent bond to L.

In some embodiments of formula (XI)-(XIIb), Z³ is optionally substituted amido, urea or thiourea. In some embodiments of formula (XI)-(XIIb), Z³ is

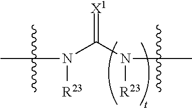

wherein:

X¹ is O or S;

t is 0 or 1; and
each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is an urea or thiourea.

In some embodiments of formula (XI)-(XIIb), Z³ is —N(R²³)SO₂— or —SO₂N(R²³)—.

In some embodiments of formula (XI)-(XIIb), Z³ is —N(R²³)CO— or —CON(R²³)—.

In some embodiments of formula (XI)-(XIIb), Z³ is —NHC(═X¹)NH—, wherein X¹ is O or S. In some embodiments, X¹ is O. In some embodiments, X¹ is S.

In some embodiments of formula (XI)-(XIIb), —Ar—Z³— is selected from:

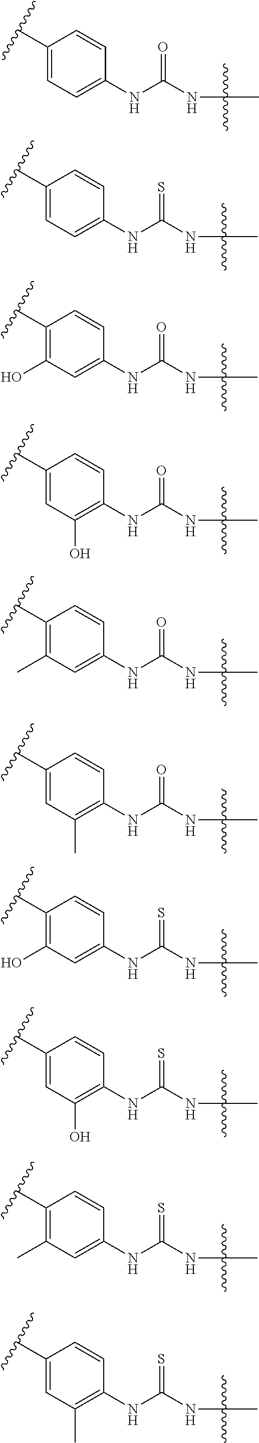

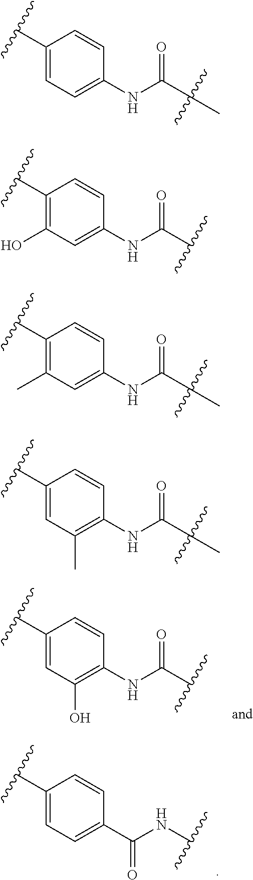 [SMILES: *C(=O)Nc1ccc(*)cc1 ; *C(=O)Nc1ccc(*)c(O)c1 ; *C(=O)Nc1ccc(*)c(C)c1 ; *C(=O)Nc1ccc(*)cc1C ; *C(=O)Nc1ccc(*)cc1O ; *NC(=O)c1ccc(*)cc1]

In some embodiments of formula (XI)-(XIIb), Z³ is optionally substituted triazole. When Z³ is optionally substituted triazole, it can be synthetically derived from click chemistry conjugation of an azido containing precursor and an alkyne containing precursor of the compound. Accordingly, in some embodiments of formula (XIIa)-(XIIb), the compound is of formula (XIIc) or (XIId):

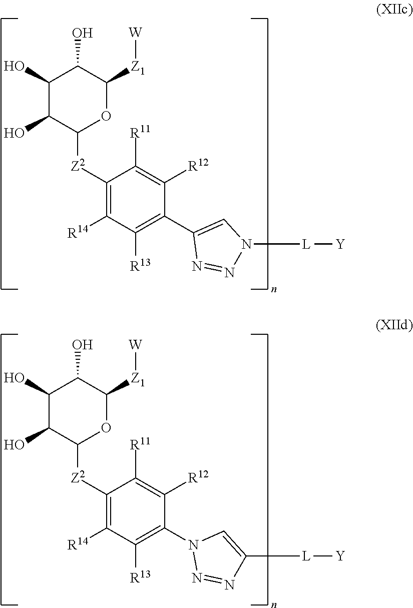

or a salt thereof, wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

In some embodiments of formula (XIIc)-(XIId), R¹¹ to R¹⁴ are each H. In some embodiments of formula (XIIc)-(XIId), at least one of R¹¹ to R¹⁴ is OH, such as 1, 2, or more of R¹¹ to R¹⁴ is OH.

In some embodiments of formula (XIIc)-(XIId), —Ar—Z³— is selected from:

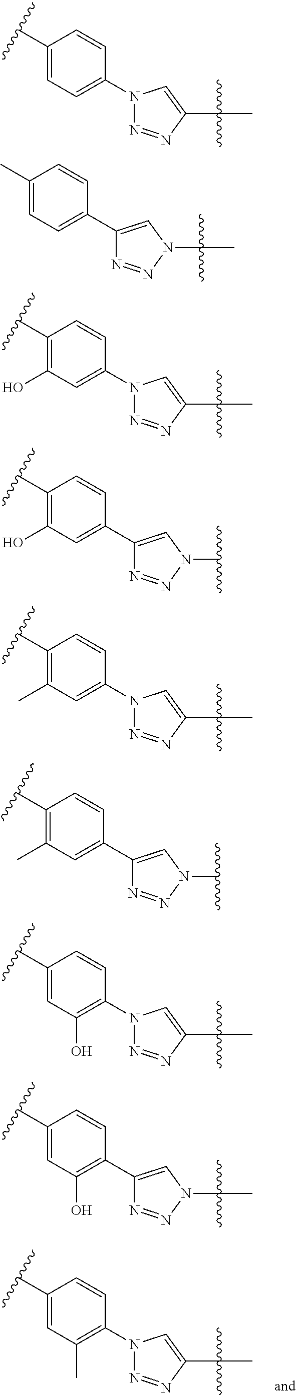

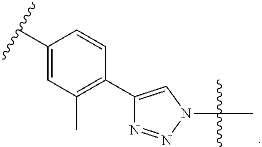 [SMILES: *c1ccc(-c2cn(*)nn2)c(C)c1]

In some embodiments of formula (XI), Ar is an optionally substituted fused bicyclic aryl or heteroaryl. In some embodiments of formula (XI), Ar is optionally substituted naphthalene or optionally substituted quinoline. In some embodiments of formula (XI), the compound is of formula (XIIIa), (XIIIb) or (XIIIb′):

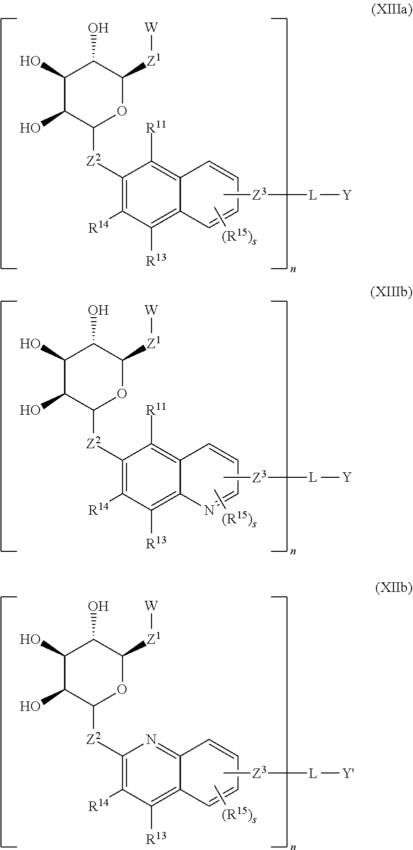

or a salt thereof, wherein:

each R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²), —OCOR²⁵, —COOR²⁵, —CONHR²⁵, and —NHCOR²⁵;

s is 0 to 3; and

each R²⁵ is independently selected from H, and optionally substituted (C₁-C₆)alkyl.

In some embodiments of formula (XIIIa)-(XIIIb′), the compound is of formula (XIIIc)-(XIIIh):

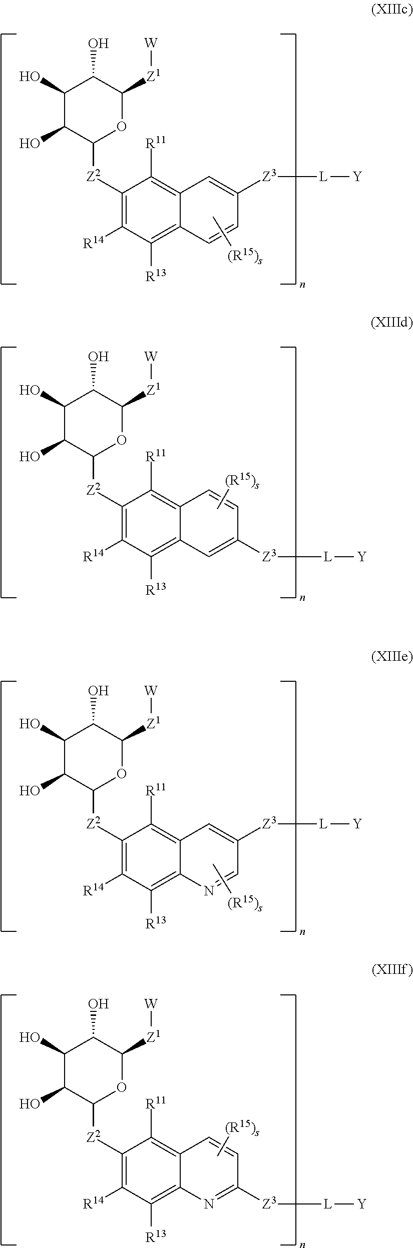

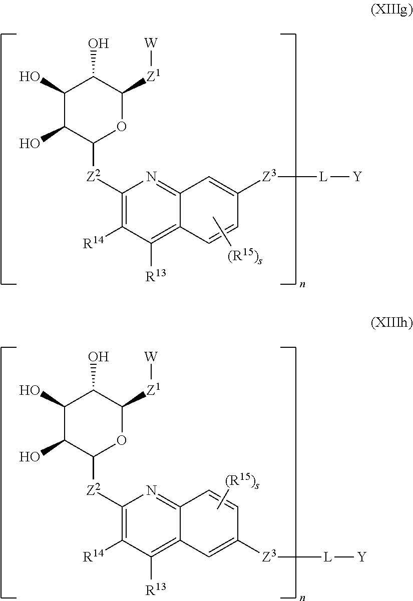

or a salt thereof.

In some embodiments of formula (XIIIa)-(XIIIh), R¹¹ to R¹⁴ are each H and s is 0. In some embodiments of formula (XIIIa)-(XIIIh), at least one of R¹¹ to R¹⁵ is OH, such as 1, 2, or more of R¹¹ to R¹⁵ is OH.

In some embodiments of formula (XIIIa)-(XIIIh), Z³ is selected from a covalent bond, —O—, —NR²³—, —NR²³CO—, —CONR²³—, —NR²³CO₂—, —OCONR²³—, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (XIIIa)-(XIIIh), Z³ is a covalent bond to L.

In some embodiments of formula (XIIIa)-(XIIIh), Z³ is optionally substituted amido, urea or thiourea. In some embodiments of formula (XIIIa)-(XIIIh), Z³ is

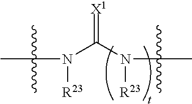

wherein:

X¹ is O or S:

t is 0 or 1; and

each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is O and X¹ is O, such that Z³ is amido. In some embodiments of Z³, t is 1 such that Z³ is an urea or thiourea.

In some embodiments of formula (XIIIa)-(XIIIh), Z³ is —N(R²³)SO₂— or —SO₂N(R²³)—.

In some embodiments of formula (XIIIa)-(XIIIh), Z³ is —N(R²³)CO— or —CON(R²³)—.

In some embodiments of formula (XIIIa)-(XIIIh), Z³ is —NHC(═X¹)NH—, wherein X¹ is O or S. In some embodiments, X¹ is O. In some embodiments, X¹ is S.

In some embodiments of formula (XIIIa)-(XIIIh), Z³ is optionally substituted triazole. When Z³ is optionally substituted triazole, it can synthetically derived from click chemistry conjugation of an azido containing precursor and an alkyne containing precursor of the compound.

In some embodiments of formula (XIIIa)-(XIIIh), —Ar—Z³— is selected from:

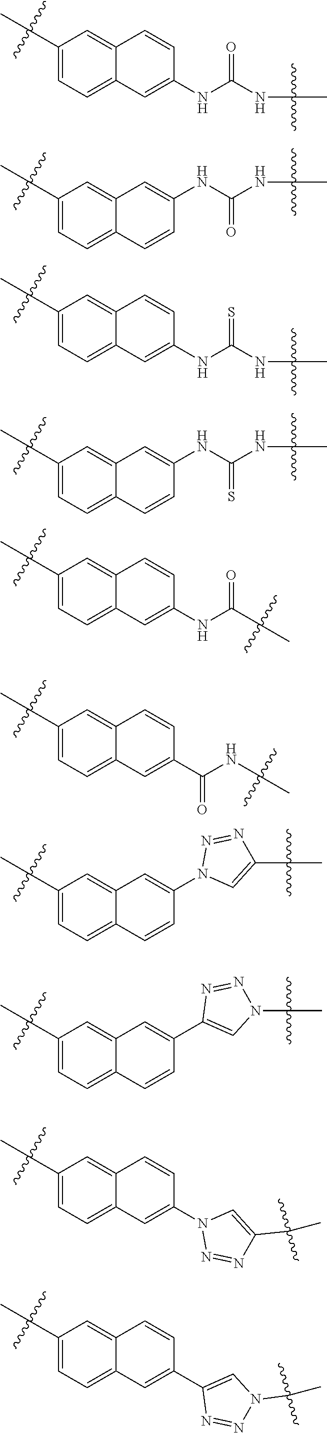

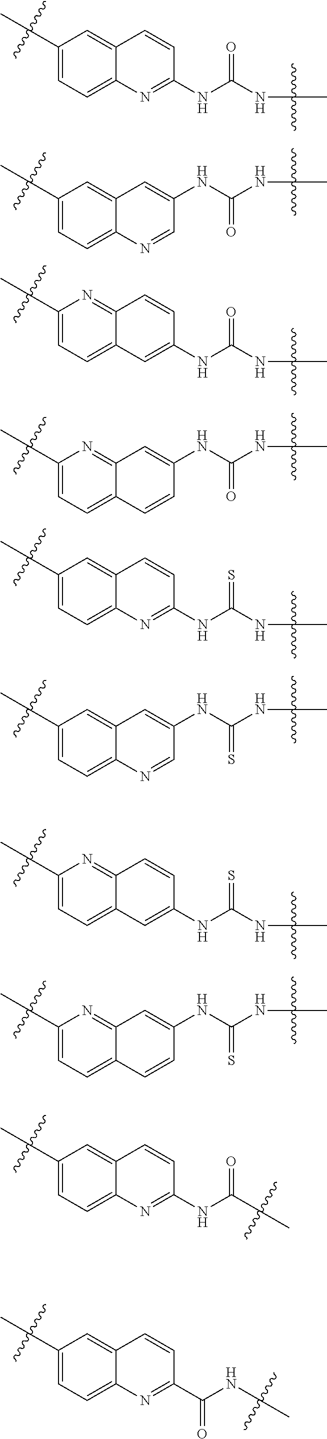

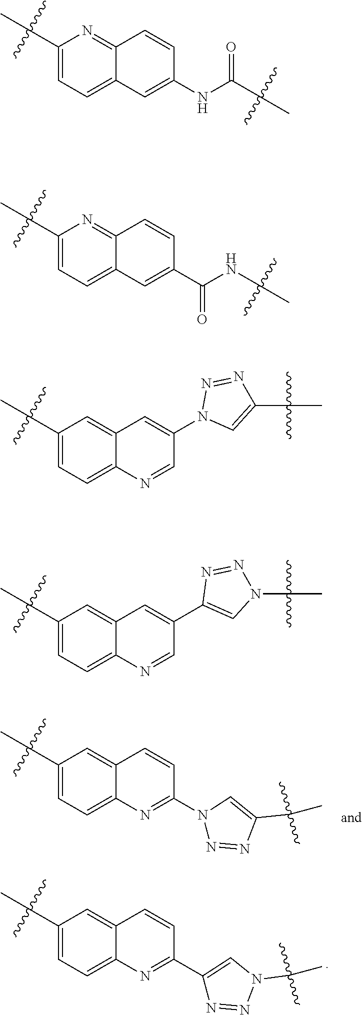 [SMILES: *C(=O)Nc1ccc2nc(*)ccc2c1 ; *NC(=O)c1ccc2nc(*)ccc2c1 ; *c1ccc2ncc(-n3cc(*)nn3)cc2c1 ; *c1ccc2ncc(-c3cn(*)nn3)cc2c1 ; *c1ccc2nc(-n3cc(*)nn3)ccc2c1 ; *c1ccc2nc(-c3cn(*)nn3)ccc2c1]

in some embodiments of formula (XI), Ar is optionally substituted bicyclic aryl or optionally substituted bicyclic heteroaryl and wherein the compound is of formula (XIVa)

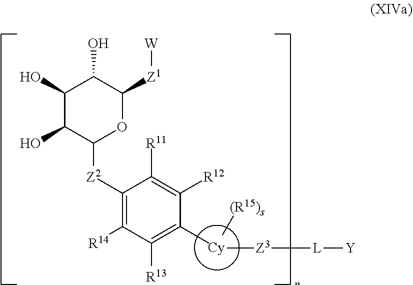

or a salt thereof, wherein:

each Cy is independently monocyclic aryl or monocyclic heteroaryl;

each R¹¹ to R¹⁵ is independently selected from H, halogen. OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

s is to 4; and

each R²⁵ is independently selected from H, and optionally substituted (C₁-C₆)alkyl.

In some embodiments of formula (XIVa), Ar is optionally substituted biphenyl. Cy is optionally substituted phenyl, and the compound is of formula (XIVb):

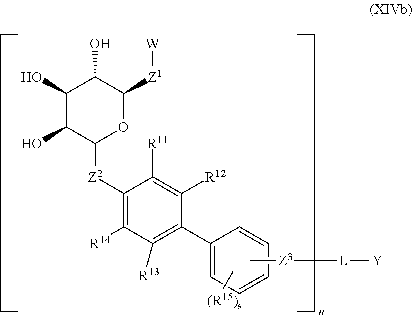

or a salt thereof.

In some embodiments of formula (XIVb), the compound is of formula (XIVc) or (XIVd):

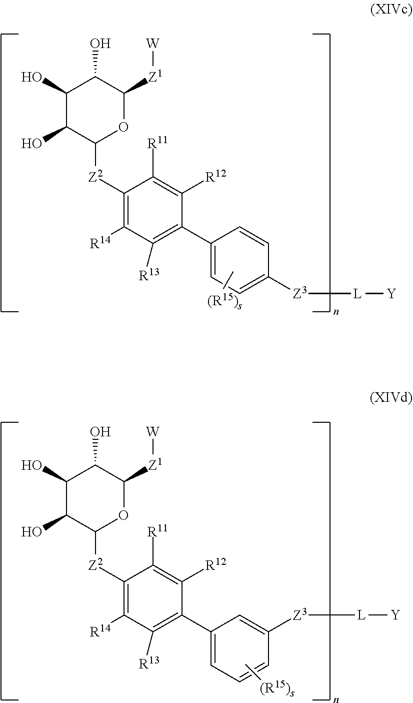

or a salt thereof.

In some embodiments of formula (XI)-(XIVd), Ar is substituted with at least one OH substituent. In some embodiments of formula (XI)-(XIVd), R¹¹ to R¹⁵ are each H. In some embodiments of formula (XI)-(XIVd), at least one of R¹¹ to R¹⁵ is OH, such as 1, 2, or more of R¹¹ to R¹⁵ is OH.

In some embodiments of formula (XI)-(XIVd), Z³ is selected from a covalent bond, —O—, —NR²³—, —NR²³CO—, —CONR²³—, R²³CO₂—, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²¹; and R²³ and R²⁴ are independently selected from H, C₍₁₋₃₎-alkyl (e.g., methyl) and substituted C₍₁₋₃₎-alkyl.

In some embodiments of formula (XI)-(XIVd), Z³ is a covalent bond to L.

In some embodiments of formula (XI)-(XIVd), Z³ is optionally substituted amido, urea or thiourea. In some embodiments of formula (XI)-(XIVd), Z³ is

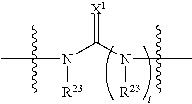

wherein:

X¹ is O or S:

t is 0 or 1; and

each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is an urea or thiourea.

In some embodiments of formula (XI)-(XIVd), Z³ is —NHC(═X¹)NH—, wherein X¹ is O or S. In some embodiments, X¹ is O. In some embodiments, X¹ is S.

In some embodiments of formula (XI)-(XIVd), Z³ is —N(R²³)SO₂— or —SO₂N(R²³)—.

In some embodiments of formula (XI)-(XIVd), Z³ is optionally substituted triazole. When Z³ is optionally substituted triazole, it can be synthetically derived from click chemistry conjugation of an azido containing precursor and an alkyne containing precursor of the compound.

In some embodiments of formula (XI)-(XIVd), —Ar—Z³— is selected from:

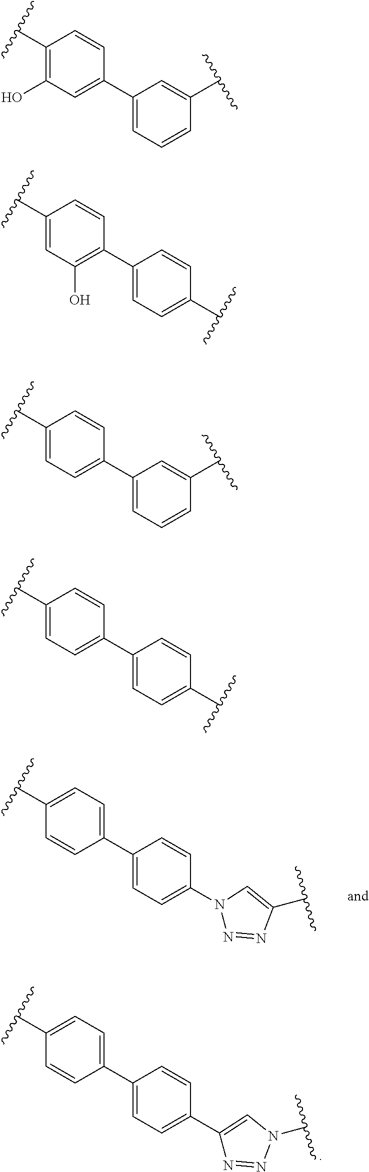 [SMILES: *c1cccc(-c2ccc(*)c(O)c2)c1 ; *c1ccc(-c2ccc(*)cc2O)cc1 ; *c1ccc(-c2cccc(*)c2)cc1 ; *c1ccc(-c2ccc(*)cc2)cc1 ; *c1ccc(-c2ccc(-n3cc(*)nn3)cc2)cc1 ; *c1ccc(-c2ccc(-c3cn(*)nn3)cc2)cc1]

In some embodiments of formula (XI), Ar is optionally substituted monocyclic heteroaryl. In some embodiments of formula (XI), Ar is triazole and wherein the compound is of formula (XVa) or (XVb):

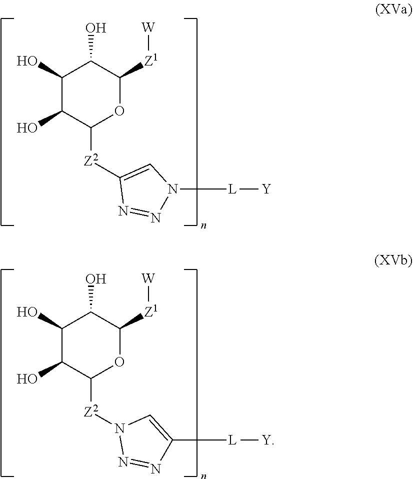

In some embodiments of formula (XVa) or (XVb), Z² is O or S. In some embodiments of formula (XVa) or (XVb), Z² is CH₂.

In some embodiments of formula (XI)-(XVb), n is at least 2, and L is a branched linker that covalently links each Ar group to Y. In some embodiments of formula (XI)-(XVb), n is 2 to 20, such as n is 2 to 10, 2 to 6, e.g., 2 or 3.

In some embodiments of formula (XI)-(XVb), n is 20 to 500 (e.g., 20 to 400, 20 to 300, or 20 to 200, or 50 to 500, or 100 to 500); and L is an a-amino acid polymer (e.g., poly-L-lysine) wherein a multitude of —Ar—Z³-groups are covalently linked to the polymer backbone via sidechain groups (e.g., via conjugation to the sidechain amino groups of lysine residues).

In some embodiments of formula (XI)-(XVb), n is at least 2 and each Z³ linking moiety is separated from every other Z³ linking moiety by a chain of at least 16 consecutive atoms via linker L, such as by a chain of at least 20, at least 25, or at least 30 consecutive atoms, and in some cases by a chain of up to 100 consecutive atoms.

In some embodiments of formula (XI)-(XVb), the compound is of formula (XVI):

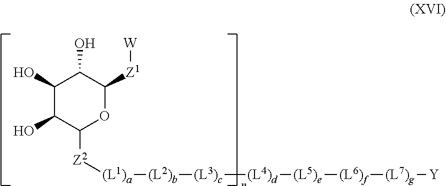

or a salt thereof, wherein:

n is 1 to 500;

each L¹ to L⁷ is independently a linking moiety that together provide a linear or branched linker between the n Z² groups and Y, and wherein -(L¹)ₐ- comprises the linking moiety Ar that is optionally substituted aryl or heteroaryl group;

a is 1 or 2; and

b, c, d, c, f, and g are each independently 0, 1, or 2.

In some embodiments of formula (XVI), the linear or branched linker separates each Z² and Y by a chain of at least 16 consecutive atoms, such as at least 20 consecutive atoms, at least 30 consecutive atoms, or 16 up to 100 consecutive atoms.

In some embodiments of formula (XVI), n is 1 to 20, such as 1 to 10, 1 to 6 or 1 to 5. In some embodiments of formula (XVI), n is at least 2, e.g., n is 2 or 3. In some embodiments of formula (XVI), when d is >0, L⁴ is a branched linking moiety that is covalently linked to each L¹ linking moiety.

In some embodiments of formula (XVI), the compound is of formula (XVIa)

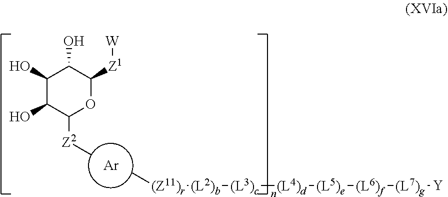

wherein:

Ar is an optionally substituted aryl or heteroaryl group;

Z¹¹ is a linking moiety;

r is 0 or 1; and

n is 1 to 6.

In some embodiments of formula (XVIa), Z¹¹ is a covalent bond, heteroatom, group having a backbone of 1-3 atoms in length (e.g., —NH—, urea, thiourea, ether, amido) or triazole.

In some embodiments of formula (XVIa), Ar is a monocyclic aryl or heteroaryl group. In some embodiments of formula (XVIa), Ar is a bicyclic aryl or heteroaryl group. In some embodiments of formula (XVIa), Ar is a tricyclic aryl or heteroaryl group. In some embodiments of formula (XVIa), Ar is selected from optionally substituted phenyl, optionally substituted biphenyl, optionally substituted naphthalene, optionally substituted triazole, optionally substituted phenyl-triazole, optionally substituted biphenyl-triazole, and optionally substituted naphthalene-triazole. In certain embodiments, Ar is optionally substituted 1,4-phenylene.

In some embodiments of formula (XVIa), Ar substituted with at least one hydroxy.

In some embodiments of formula (XVI)-(XVIa), L¹ or —Ar—(Z¹¹)ᵣ— is selected from:

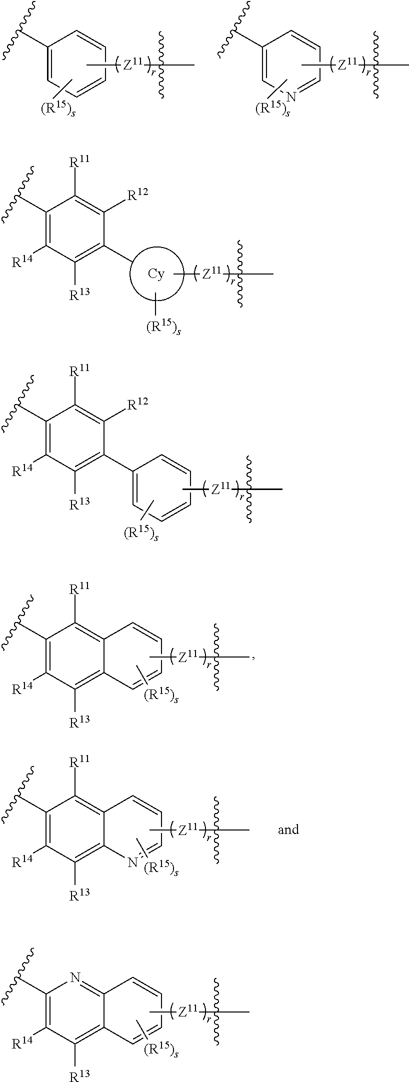

wherein:

Cy is monocyclic aryl or heteroaryl;

r is 0 or 1;

s is 0 to 4 (e.g., 0 to 3, or 0, 1 or 2);

R¹¹ to R¹⁴ and each R¹⁵ are independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵ and —NHCOR²⁵, wherein each R²⁵ is independently selected from H, C₍₁₋₆₎-alkyl and substituted C₍₁₋₆₎-alkyl; and

Z¹¹ is selected from covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X²— and optionally substituted triazole, where X¹ and X² are selected from O, S and NR¹³, wherein R²³ and R²⁴ are independently selected from H, C₍₁₋₃₎-alkyl (e.g., methyl) and substituted C₍₁₋₃₎-alkyl.

In some embodiments, r is 0 and Z¹¹ is absent. In some embodiments, r is 1.

In some embodiments of formula (XVI)-(XVIa), L¹ or —Ar—(Z¹¹)ᵣ— is

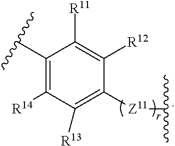

In some embodiments, r is 0 and Z¹¹ is absent. In some embodiments, r is 1.

In some embodiments of formula (XVI)-(XVIa), L¹ or —Ar—(Z¹¹)ᵣ— is

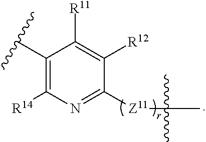

In some embodiments, r is 0 and Z¹¹ is absent. In some embodiments, r is 1.

In some embodiments of formula (XVI)-(XVIa), L¹ or —Ar—(Z¹¹)ᵣ— is

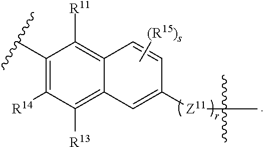

In some embodiments, r is 0 and Z¹¹ is absent. In some embodiments, r is 1.

In some embodiments of formula (XVI)-(XVIa), L¹ or —Ar—(Z¹¹)ᵣ— is

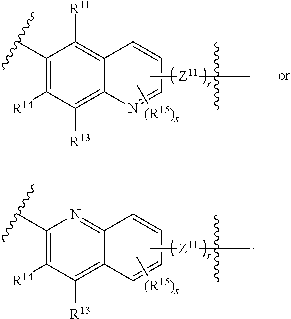

In some embodiments, r is 0 and Z¹¹ is absent. In some embodiments, r is 1.

In some embodiments of formula (XVI)-(XVIa), L¹ or —Ar—(Z¹¹)ᵣ— is selected from:

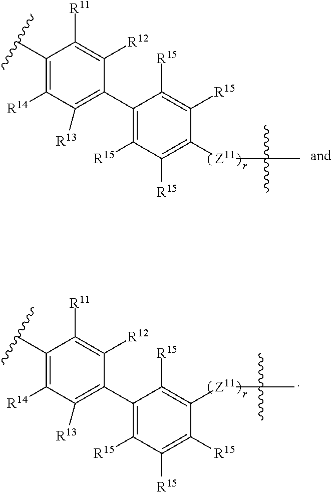

In some embodiments, r is 0 and Z¹¹ is absent. In some embodiments, r is 1 and Z¹¹ is selected from —O—, —NR²³—, —NR²³CO—, CONR²³—, —NR²³CO₂—, —OCONR²³—, —NR²³C(═X¹)NR²³—, —CR²⁴═N—, —CR²⁴═N—X²—, —NR²³SO₂—, and —SO₂NR²³—; wherein X¹ and X² are selected from O, S and NR²³, and each R²³ and R²⁴ is independently selected from H, C₍₁₋₃₎-alkyl (e.g., methyl) and substituted C₍₁₋₃₎-alkyl.

In some embodiments, r is 1 and Z¹¹ is

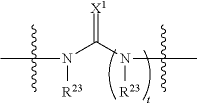

wherein:

X¹ is 0 or S;
t is 0 or 1; and

each R²³ is independently selected from H, C₍₁₋₃₎-alkyl (e.g., methyl) and substituted C₍₁₋₃₎-alkyl. In some embodiments, Z¹¹ is —NHC(═X¹)NH—, wherein X¹ is O or S. In some embodiments, r is 1 and Z¹¹ is triazole.

In some embodiments of formula (XI)-(XVIa), Z³ is —N(R²³)SO₂— or —SO₂N(R²³)—.

In some embodiments of formula (XI)-(XVIa), Z³ is —N(R²³)CO— or —CON(R²³)—.

In some embodiments of formula (XI)-(XVIa), the hydrophilic head group W is charged, e.g., capable of forming a salt under aqueous or physiological conditions. In some embodiments of formula (XI)-(XVIa), the hydrophilic head group W is neutral.

In any one of the embodiments of formula (XI)-(XVIa) described herein, the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)₂H, —NHSO₂NHR³, —NHC(O)NHS(O)₂R³, —NHSO₂R³, —NHSO₃H,

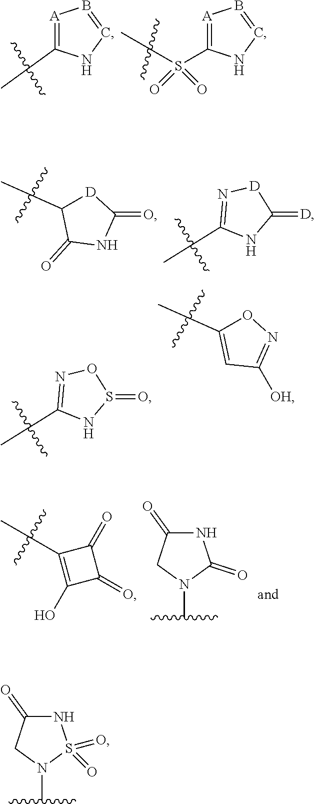

or a salt thereof, wherein:

R¹ and R² are independently hydrogen, SR³, halo, or CN, and R³ and R⁴ are independently H, C₁0.6 alkyl or substituted C₁₋₆-alkyl (e.g., —CF₃ or —CH₂CF₃);

A, B, and C are each independently CH or N; and

D is each independently O or S.

In some embodiments of formula (XI)-(XVIa), the hydrophilic head group W is phosphate or thiophosphate (e.g., —OP═O(OH)₂, —SP═O(OH)₂, —OP═O(SH)(OH), —SP═O(SH)(OH), or —OP═S(OH)₂). In some embodiments of formula (XI)-(XVIa), the hydrophilic head group W is phosphonate or thiophosphonate (e.g., —P═O(OH)₂, —P═S(OH)₂, —P═O(SH)(OH), or —P═S(SH)(OH), or a salt thereof). In some embodiments of formula (XI)-(XVIa), the hydrophilic head group W is sulfonate (e.g., —SO₃H or a salt thereof). In some embodiments of formula (XI)-(XVIa), the hydrophilic head group W is —CO₂H or a salt thereof. In some embodiments of formula (XI)-(XVIa), the hydrophilic head group W is malonate (e.g., —CH(COOH)₂ or a salt thereof).

In some embodiments of formula (XI)-(XVIa), the hydrophilic head group W comprises a 5-membered heterocycle, such as

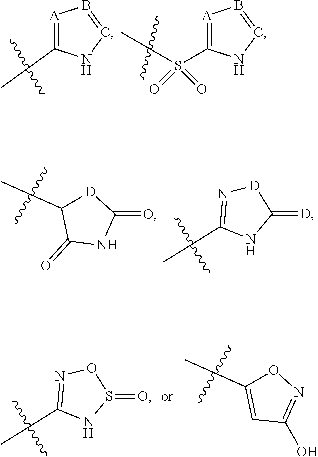

or a salt thereof.

Exemplary hydrophilic head group W are shown in the X groups of Table 1, and the compound Tables.

In some embodiments of formula (XI)-(XVIa), the linking moiety (Z¹) that connects the hydrophilic head group W to the mannose ring is —(CH₂)ⱼ— where j is 1-3. In some embodiments, j is 2. In some embodiments of formula (XI)-(XVIa), the linking moiety (Z¹) that connects the hydrophilic head group W to the mannose ring is —CH═CH.

In some embodiments of formula (XI)-(XVIa), the linking moiety (Z²) that connects the mannose ring to the Ar group is O or S. In some embodiments of formula (XI)-(XVIa), Z² is —NR²¹—, where R²¹ is selected from H. and optionally substituted (C₁-C₆)alkyl. In some embodiments of formula (XI)-(XVIa), Z² is —NH—. In some embodiments of formula (XI)-(XVIa), Z² is —C(R²²)₂—, where each R²² is independently selected from H, halogen (e.g., F) and optionally substituted (C₁-C₆)alkyl. In some embodiments of formula (XI)-(XVIa), Z² is CH₂. In some embodiments of formula (XI)-(XVIa), Z² is —CF₂— or —C(CH₃)₂—.

In some embodiments of formula (XI)-(XVIa), Z¹ is selected from —(CH₂)ⱼ— and —CH═CH—; j is 1 to 3; and Z² is selected from O and CH₂.

In some embodiments of formula (XI)-(XVIa), Z¹ is —(CH₂)ⱼ—; j is 2; and Z² is O.

In some embodiments of formula (XI)-(XVIa), Z¹ is —(CH₂)ⱼ—; j is 2; and Z² is CH₂.

In some embodiments of formula (XI)-(XVIa), Z¹ is —CH═CH—; and Z² is O.

In some embodiments of formula (XI)-(XVIa), Z¹ is —CH═CH—; and Z² is CH₂.

As summarized above, the M6PR binding moiety (X) of the compounds of this disclosure (e.g., of formula (Ia)) can include a mannose ring or analog thereof described by the following structure:

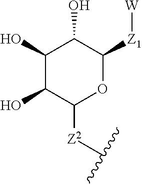

where:

W is a hydrophilic head group:

Z¹ is selected from optionally substituted (C₁-C₃)alkylene and optionally substituted ethenylene;

Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

The mannose ring or analog thereof of the M6PR binding moiety can be incorporated into the compounds of this disclosure by attachment to the Z² group via a linking moiety. It is understood that in the compounds of formula (Ia), the group or linking moiety attached to Z² can, in some cases, be considered to be part of the M6PR binding moiety (X) and provide for desirable binding to the M6PR. See e.g., formula (XI)-(XVIa), where an aryl or heteroaryl linking moiety is attached to the mannose ring or analog via the Z² group. In certain other cases, the group or linking moiety attached to Z² can be considered part of the linker L of formula (Ia).

In some embodiments of the M6PR binding compounds of this disclosure, e.g., a compound of formula (Ia), the M6PR binding moiety X comprises the group of formula (IIIa), (IIIb), (IIc), or (IIId):

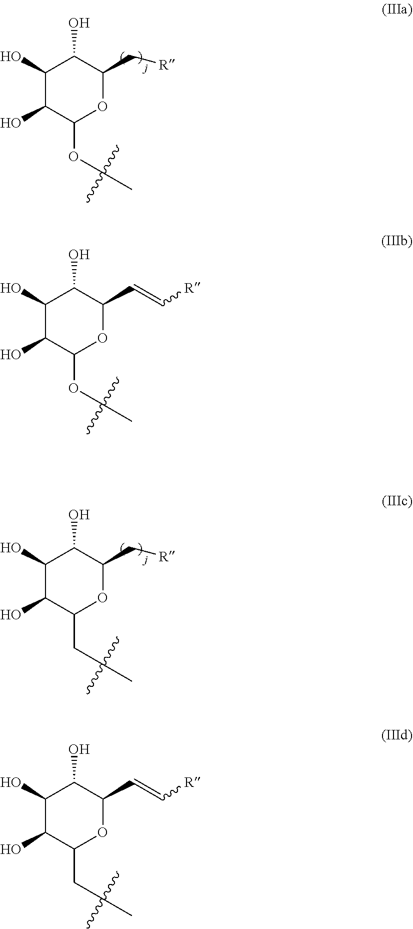

wherein R″ (e.g., a hydrophilic head group) is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R, —NHSO₂R³,

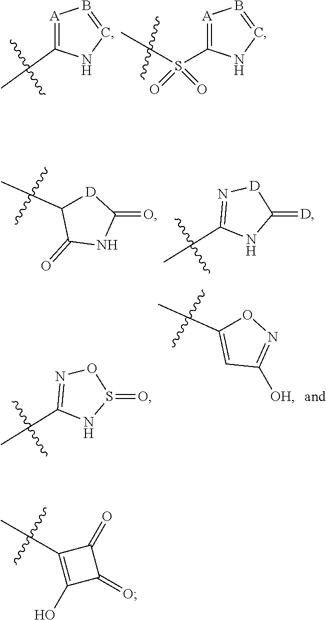

wherein j is an integer of 1 to 3;
wherein R¹ and R² are each independently hydrogen, halo, or CN;
wherein R³ and R⁴ are each independently C₁₋₆ alkyl; and
wherein A, B, and C are each independently CH or N; and D is each independently O or S.

In some embodiments of formula (IIIa), (IIIb), (IIIc), or (IIId), R″ is selected from the group consisting of —OH, —CR¹R²OH, —P═O(OH)₂, P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

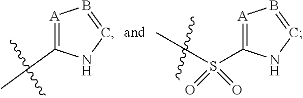

j is an integer of 1 to 3;

R¹ and R² are each independently hydrogen, halo, or CN;

R³ and R⁴ are each independently C₁₋₆ alkyl; and

A, B, and C are each independently CH or N.

In certain embodiments, X comprises the group of formula (IIIa′), (IIIa″), (IIIb′), (IIIb″), (IIIc′), (IIIc″), (IIId′) or (IIId″):

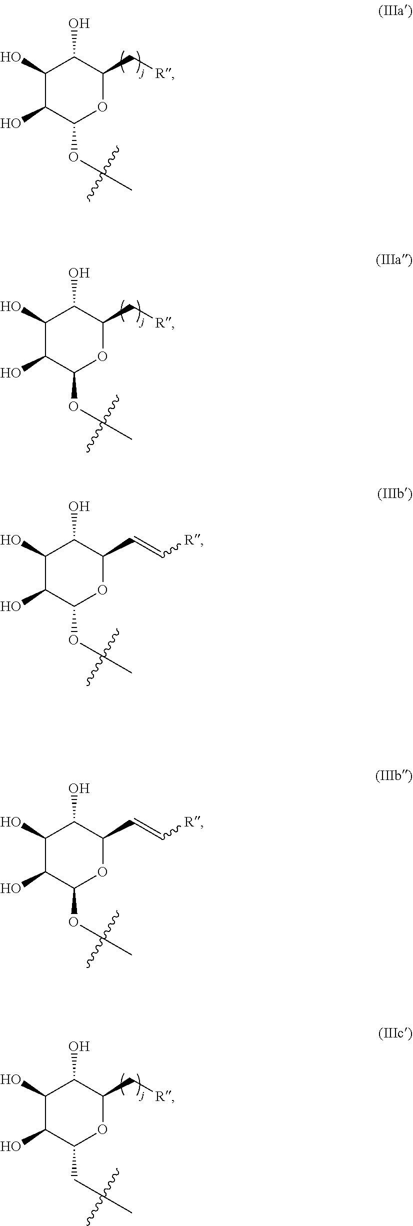

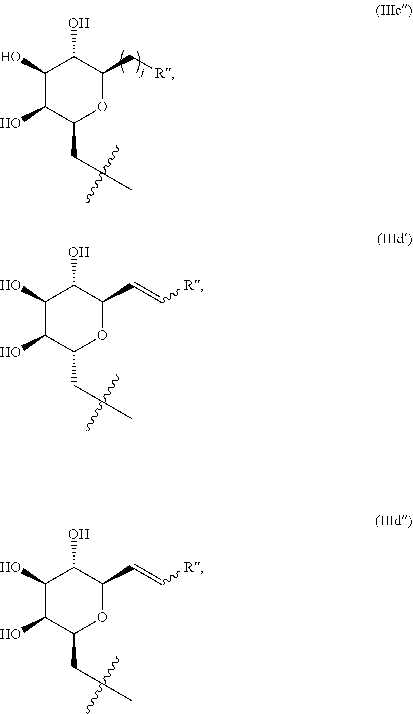

wherein R″ is as defined herein and wherein j is an integer of 1 to 3.

In certain embodiments, X is of formula (IIIa′), (IIIa″), (IIIb′), or (IIIb″). In certain embodiments, X is of formula (IIIc′), (IIIc″), (IIId′) or (IIId″). In certain embodiments, X is of formula (IIIa′) or (IIIa″). In certain embodiments, X is of formula (IIIb′) or (IIIb″). In certain embodiments, X is of formula (IIIc′) or (IIIc″). In certain embodiments, X is of formula (IIId′) or (IIId″). In certain embodiments, X is of formula (IIIa′). In one embodiment, X is of formula (IIIa″). In certain embodiments, X is of formula (IIIb′). In one embodiment, X is of formula (IIIb″). In certain embodiments, X is of formula (IIIc′). In one embodiment, X is of formula (IIIc″). In certain embodiments. X is of formula (IIId′). In one embodiment, X is of formula (IIId″). In certain embodiments, X is of formula (IIIe).

In one embodiment, j is 1 or 2. In another embodiment, j is 2 or 3. In another embodiment, j is 1. In another embodiment, j is 2. In yet another embodiment, j is 3.

In certain embodiments, R″ is selected from the group consisting of —OH, —CR¹R²OH, —P═O(OH)₂, P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

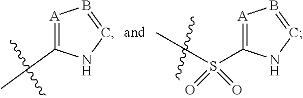

wherein R¹ and R² are each independently hydrogen, halo, or CN;
wherein R³ and R⁴ are each independently C₁₋₆ alkyl; and

wherein A, B, and C are each independently CH or N. In certain embodiments. R″ is not OH.

In certain embodiments, R″ is selected from the group consisting of —OH, —CR¹R²OH, —P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

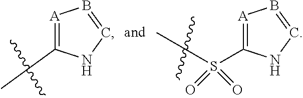

In certain embodiments, R″ is selected from the group consisting of —CR¹R²OH, —P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

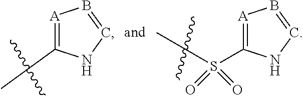

In certain embodiments, R″ is selected from the group consisting of —P═O(OH)₂, P(═O)R¹OH, and —(CR¹R²)—P═O(OH)₂. In certain embodiments, R″ is selected from the group consisting of —SO₂OH, —OSO₂OH, —CONHSO₂R³, —SO₂R³, —SOR³R⁴, —SO₂NH₂, —SO₂NHR³, —SO₂NR³R⁴, and —NHSO₂R³. In certain embodiments, R″ is —OH, or —CR¹R²OH. In certain embodiments, R″ is selected from the group consisting of —COOH, —CONH₂, —CONHR¹, —CONR³R⁴, —CH(COOH)₂, —CR¹R²COOH, and —NHCOR³.

In certain embodiments of formula (Ia), X comprises the group of formula (IIIa-1) or (IIIb-1):

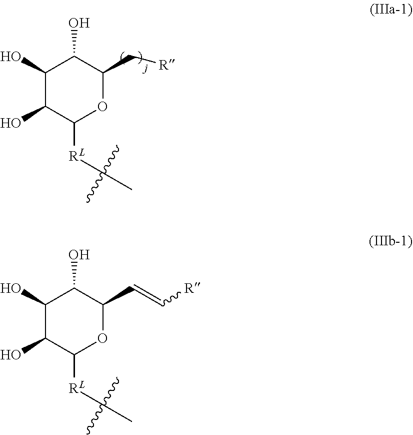

wherein:

R^{L} is —O—, —NH— or —CH₂—;

R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

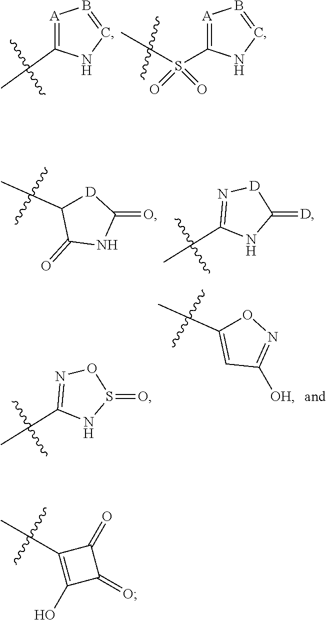

j is an integer of 1 to 3;

R¹ and R² are each independently hydrogen, halo, or CN;

R³ and R⁴ are each independently C₁₋₆ alkyl;

A, B, and C are each independently CH or N; and

D is each independently 0 or S.

In certain embodiments of formula (Ia), wherein X is of formula (IIIa-1) or (IIIb-1), when R^{L} is —O—, R″ is

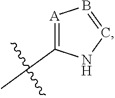

and B and C are N, then j is 2.

In certain embodiments of formula (Ia), wherein X is of formula (IIIa-1) or (IIIb-1), when R^{L} is —O— and R″ is —CR¹R²COOH, R¹ and R² are not both hydrogen.

In certain embodiments of formula (Ia), wherein X is of formula (IIIa-1) or (IIIb-1), when R^{L} is —O—, R″ is

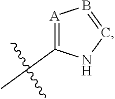

and B and C are N, then j is 2; and when R^{L} is —O— and R″ is —CR¹R²COOH, R¹ and R² are not both hydrogen.

In certain embodiments of the formula (Ia), X is of formula (IIIa-1) or (IIIb-1), R^{L} is —NH— or —CH₂— and R″ and the remaining variables are as described for formula (Ia).

In certain embodiments of the formula (Ia), X is of formula (IIIa-1) or (IIIb-1), and when R′ is —O—, R″ is

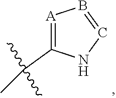

and B and C are N, then j is 2 and provided when R′ is —O—, R″ is —CR¹R²COOH, R¹ and R² are not both hydrogen.

In certain embodiments, provided herein are compounds of the formula (Ia), wherein X is of formula (IIIa-1) or (IIIb-1), wherein R′ is —O—, —NH— or —CH₂— and R″ is selected from the group consisting of —P═O(OH)₂, P(═O)R¹OH, —PH(═O)OH, —(CR¹R²)P═O(OH)₂, —S(O)OH, —OSO₂OH, —CONH(OH), —CONH(OR³)—CONHSO₂R³, —CONHSO₂NR³R⁴, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R²,

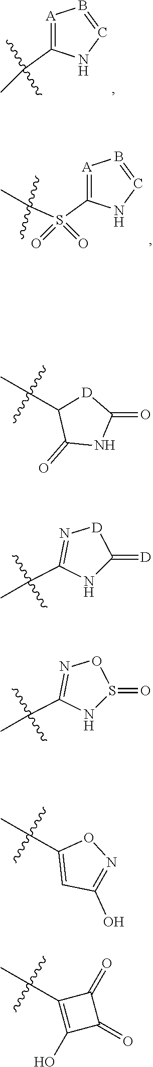

and the remaining variables are as described for formula (Ia).

Exemplary moieties that bind the M6PR (X1 to X27), and synthons which ca be utilized in the preparation of compounds and conjugates of this disclosure that include the M6PR ligand of interest are shown in Table 1. It is understood that a variety of chemoselective ligation groups can be utilized in place of the exemplary groups shown (e.g., Click chemistry group) in the exemplary synthetic precursors of Table 1. For example, the alkyne containing precursors can be conjugated to a linker having a cysteine reactive or lysine reactive chemoselective ligation group suitable for conjugation to a viral composition, or can be replaced with such a suitable cysteine reactive or lysine reactive chemoselective ligation group.

TABLE 1
Exemplary M6PR binding ligands (X)

| Exemplary X for M6PR binding compounds | | |
|---|---|---|
| # | Structure | Exemplary Synthetic precursors |
| X1 | 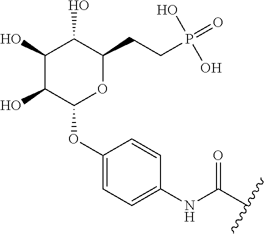 [SMILES: *C(=O)Nc1ccc(O[C@H]2O[C@H](CCP(=O)(O)O)[C@@H](O)[C@H](O)[C@@H]2O)cc1] | 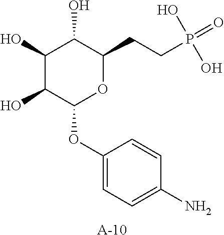 |
| X2 | 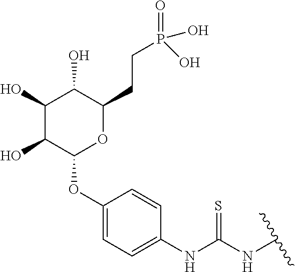 [SMILES: *NC(=S)Nc1ccc(O[C@H]2O[C@H](CCP(=O)(O)O)[C@@H](O)[C@H](O)[C@@H]2O)cc1] | 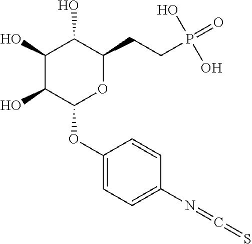 [SMILES: O=P(O)(O)CC[C@H]1O[C@H](Oc2ccc(N=C=S)cc2)[C@@H](O)[C@@H](O)[C@@H]1O] |
| | | 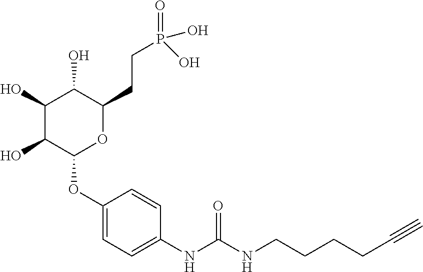 [SMILES: C#CCCCCNC(=O)Nc1ccc(O[C@H]2O[C@H](CCP(=O)(O)O)[C@@H](O)[C@H](O)[C@@H]2O)cc1] |
| X3 | 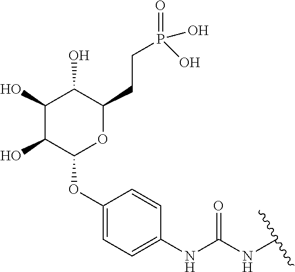 [SMILES: *NC(=O)Nc1ccc(O[C@H]2O[C@H](CCP(=O)(O)O)[C@@H](O)[C@H](O)[C@@H]2O)cc1] | 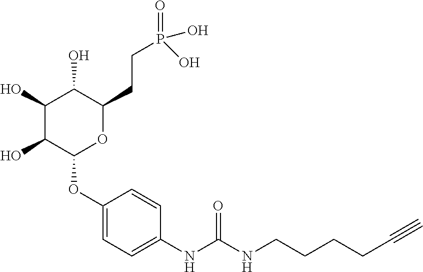 [SMILES: C#CCCCCNC(=O)Nc1ccc(O[C@H]2O[C@H](CCP(=O)(O)O)[C@@H](O)[C@H](O)[C@@H]2O)cc1] |
| X4 | 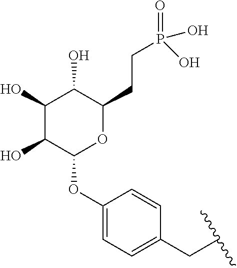 [SMILES: *Cc1ccc(O[C@H]2O[C@H](CCP(=O)(O)O)[C@@H](O)[C@H](O)[C@@H]2O)cc1] | 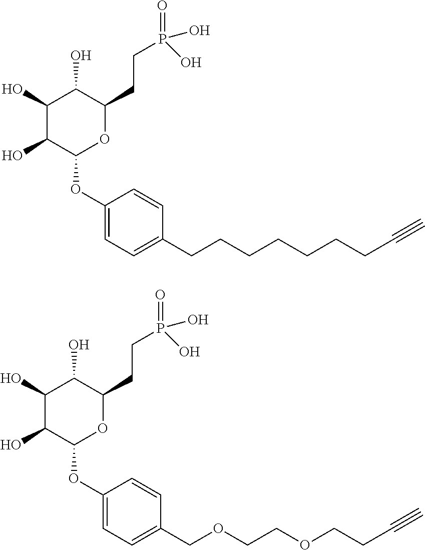 [SMILES: C#CCCCCCCCc1ccc(O[C@H]2O[C@H](CCP(=O)(O)O)[C@@H](O)[C@H](O)[C@@H]2O)cc1 ; C#CCCOCCOCc1ccc(O[C@H]2O[C@H](CCP(=O)(O)O)[C@@H](O)[C@H](O)[C@@H]2O)cc1] |
| X5 | 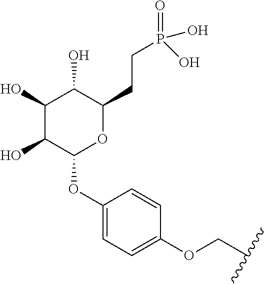 [SMILES: *COc1ccc(O[C@H]2O[C@H](CCP(=O)(O)O)[C@@H](O)[C@H](O)[C@@H]2O)cc1] | 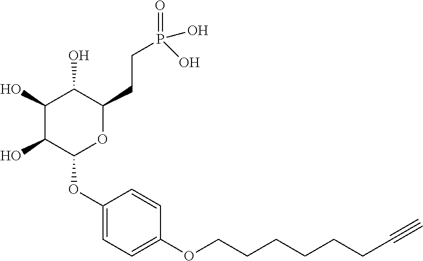 [SMILES: C#CCCCCCCOc1ccc(O[C@H]2O[C@H](CCP(=O)(O)O)[C@@H](O)[C@H](O)[C@@H]2O)cc1] |
| X6 | 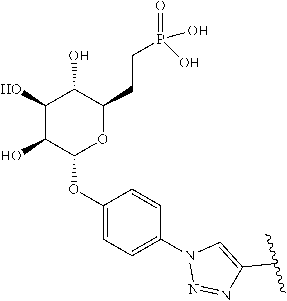 [SMILES: *c1cn(-c2ccc(O[C@H]3O[C@H](CCP(=O)(O)O)[C@@H](O)[C@H](O)[C@@H]3O)cc2)nn1] | 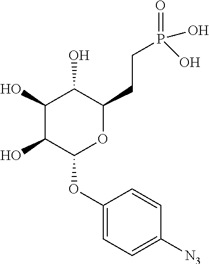 [SMILES: [N-]=[N+]=Nc1ccc(O[C@H]2O[C@H](CCP(=O)(O)O)[C@@H](O)[C@H](O)[C@@H]2O)cc1] |
| X7 | 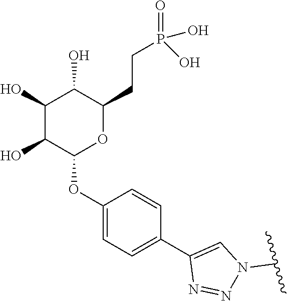 [SMILES: *n1cc(-c2ccc(O[C@H]3O[C@H](CCP(=O)(O)O)[C@@H](O)[C@H](O)[C@@H]3O)cc2)nn1] | 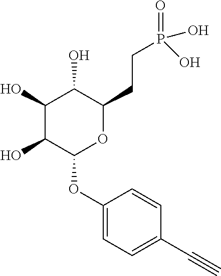 [SMILES: C#Cc1ccc(O[C@H]2O[C@H](CCP(=O)(O)O)[C@@H](O)[C@H](O)[C@@H]2O)cc1] |
| X8 | 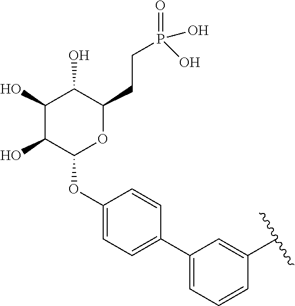 [SMILES: *c1cccc(-c2ccc(O[C@H]3O[C@H](CCP(=O)(O)O)[C@@H](O)[C@H](O)[C@@H]3O)cc2)c1] | 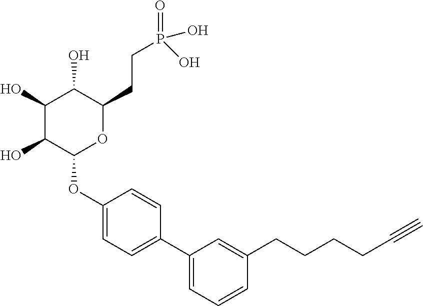 [SMILES: C#CCCCCc1cccc(-c2ccc(O[C@H]3O[C@H](CCP(=O)(O)O)[C@@H](O)[C@H](O)[C@@H]3O)cc2)c1] |
| X9 | 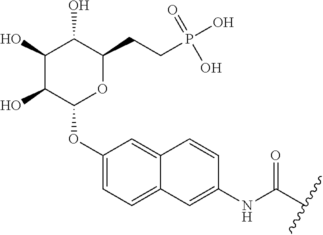 [SMILES: *C(=O)Nc1ccc2cc(O[C@H]3O[C@H](CCP(=O)(O)O)[C@@H](O)[C@H](O)[C@@H]3O)ccc2c1] | 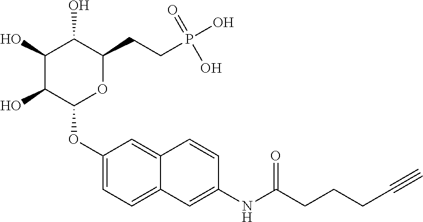 [SMILES: C#CCCCC(=O)Nc1ccc2cc(O[C@H]3O[C@H](CCP(=O)(O)O)[C@@H](O)[C@H](O)[C@@H]3O)ccc2c1] |
| X10 | 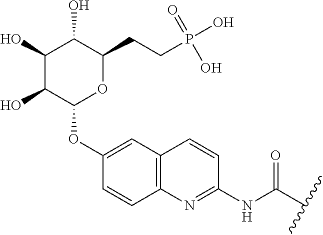 [SMILES: *C(=O)Nc1ccc2cc(O[C@H]3O[C@H](CCP(=O)(O)O)[C@@H](O)[C@H](O)[C@@H]3O)ccc2n1] | 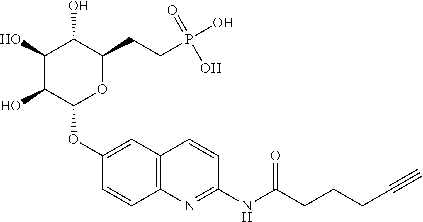 [SMILES: C#CCCCC(=O)Nc1ccc2cc(O[C@H]3O[C@H](CCP(=O)(O)O)[C@@H](O)[C@H](O)[C@@H]3O)ccc2n1] |
| X11 | 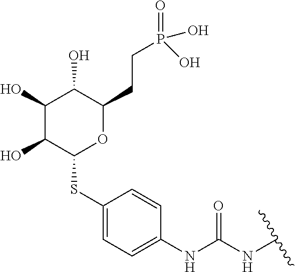 [SMILES: *NC(=O)Nc1ccc(S[C@H]2O[C@H](CCP(=O)(O)O)[C@@H](O)[C@H](O)[C@@H]2O)cc1] | 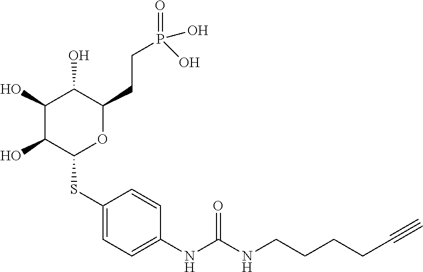 [SMILES: C#CCCCCNC(=O)Nc1ccc(S[C@H]2O[C@H](CCP(=O)(O)O)[C@@H](O)[C@H](O)[C@@H]2O)cc1] |
| X12 | 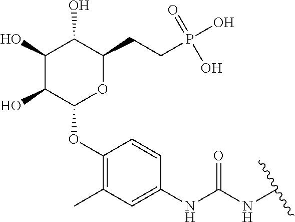 [SMILES: *NC(=O)Nc1ccc(O[C@H]2O[C@H](CCP(=O)(O)O)[C@@H](O)[C@H](O)[C@@H]2O)c(C)c1] | 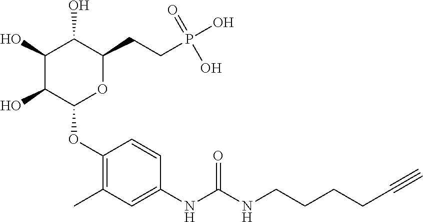 [SMILES: C#CCCCCNC(=O)Nc1ccc(O[C@H]2O[C@H](CCP(=O)(O)O)[C@@H](O)[C@H](O)[C@@H]2O)c(C)c1] |
| X13 | 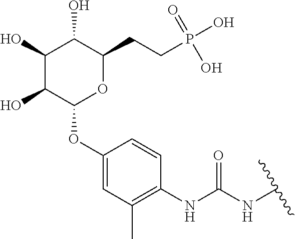 [SMILES: *NC(=O)Nc1ccc(O[C@H]2O[C@H](CCP(=O)(O)O)[C@@H](O)[C@H](O)[C@@H]2O)cc1C] | 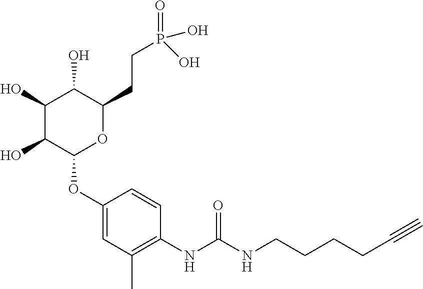 [SMILES: C#CCCCCNC(=O)Nc1ccc(O[C@H]2O[C@H](CCP(=O)(O)O)[C@@H](O)[C@H](O)[C@@H]2O)cc1C] |
| X14 | 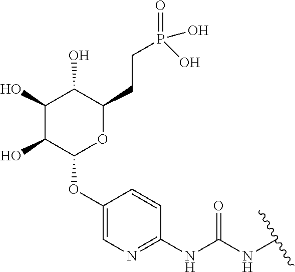 [SMILES: *NC(=O)Nc1ccc(O[C@H]2O[C@H](CCP(=O)(O)O)[C@@H](O)[C@H](O)[C@@H]2O)cn1] | 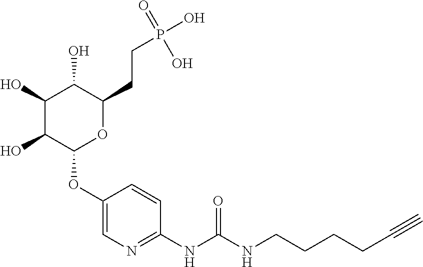 [SMILES: C#CCCCCNC(=O)Nc1ccc(O[C@H]2O[C@H](CCP(=O)(O)O)[C@@H](O)[C@H](O)[C@@H]2O)cn1] |
| X15 | 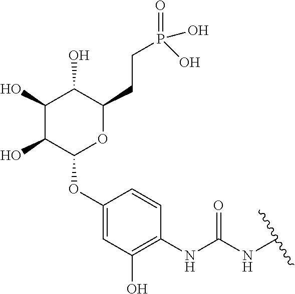 [SMILES: *NC(=O)Nc1ccc(O[C@H]2O[C@H](CCP(=O)(O)O)[C@@H](O)[C@H](O)[C@@H]2O)cc1O] | 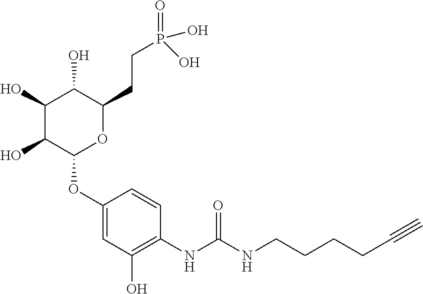 [SMILES: C#CCCCCNC(=O)Nc1ccc(O[C@H]2O[C@H](CCP(=O)(O)O)[C@@H](O)[C@H](O)[C@@H]2O)cc1O] |
| X16 | 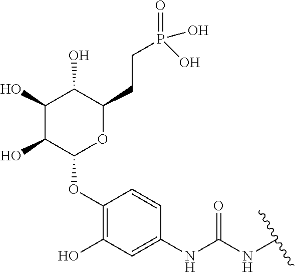 [SMILES: *NC(=O)Nc1ccc(O[C@H]2O[C@H](CCP(=O)(O)O)[C@@H](O)[C@H](O)[C@@H]2O)c(O)c1] | 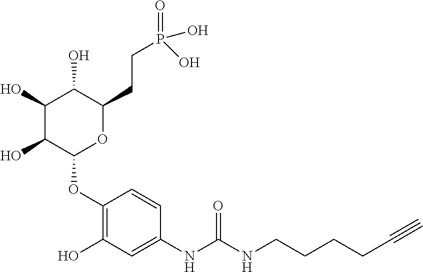 [SMILES: C#CCCCCNC(=O)Nc1ccc(O[C@H]2O[C@H](CCP(=O)(O)O)[C@@H](O)[C@H](O)[C@@H]2O)c(O)c1] |
| X17 | 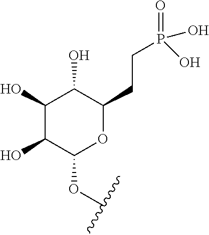 [SMILES: *O[C@H]1O[C@H](CCP(=O)(O)O)[C@@H](O)[C@H](O)[C@@H]1O] | 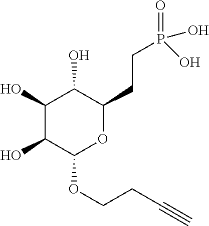 [SMILES: C#CCCO[C@H]1O[C@H](CCP(=O)(O)O)[C@@H](O)[C@H](O)[C@@H]1O] |
| X18 | 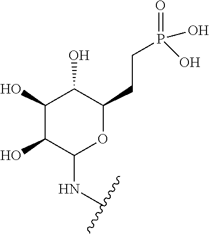 [SMILES: *NC1O[C@H](CCP(=O)(O)O)[C@@H](O)[C@H](O)[C@@H]1O] | 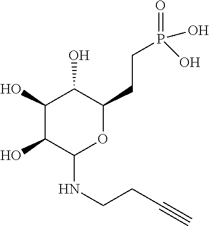 [SMILES: C#CCCNC1O[C@H](CCP(=O)(O)O)[C@@H](O)[C@H](O)[C@@H]1O] |
| X19 | 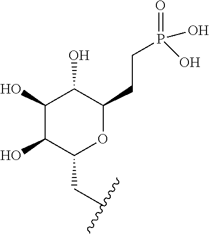 [SMILES: *C[C@H]1O[C@H](CCP(=O)(O)O)[C@@H](O)[C@H](O)[C@@H]1O] | 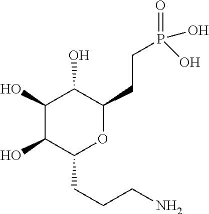 [SMILES: NCCC[C@H]1O[C@H](CCP(=O)(O)O)[C@@H](O)[C@H](O)[C@@H]1O] |
| X20 | 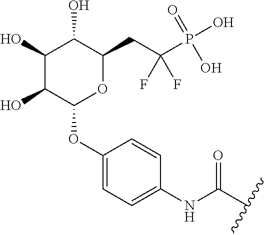 [SMILES: *C(=O)Nc1ccc(O[C@H]2O[C@H](CC(F)(F)P(=O)(O)O)[C@@H](O)[C@H](O)[C@@H]2O)cc1] | 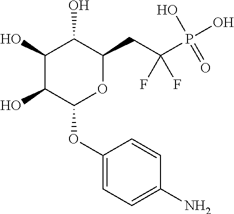 [SMILES: Nc1ccc(O[C@H]2O[C@H](CC(F)(F)P(=O)(O)O)[C@@H](O)[C@H](O)[C@@H]2O)cc1] |
| X21 | 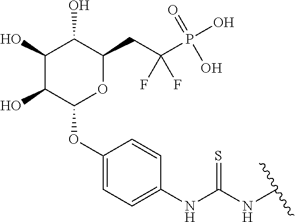 [SMILES: *NC(=S)Nc1ccc(O[C@H]2O[C@H](CC(F)(F)P(=O)(O)O)[C@@H](O)[C@H](O)[C@@H]2O)cc1] | 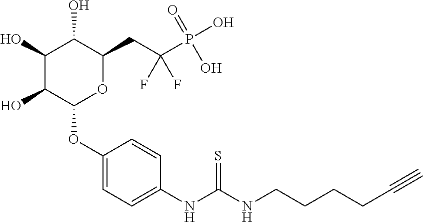 [SMILES: C#CCCCCNC(=S)Nc1ccc(O[C@H]2O[C@H](CC(F)(F)P(=O)(O)O)[C@@H](O)[C@H](O)[C@@H]2O)cc1] |
| X22 | 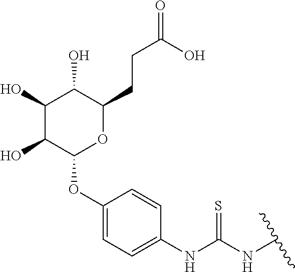 [SMILES: *NC(=S)Nc1ccc(O[C@H]2O[C@H](CCC(=O)O)[C@@H](O)[C@H](O)[C@@H]2O)cc1] | 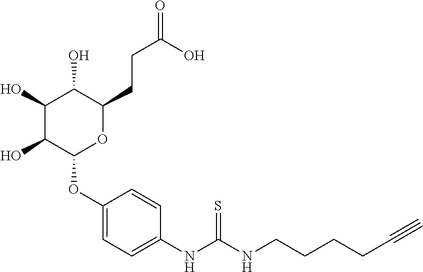 [SMILES: C#CCCCCNC(=S)Nc1ccc(O[C@H]2O[C@H](CCC(=O)O)[C@@H](O)[C@H](O)[C@@H]2O)cc1] |
| X23 | 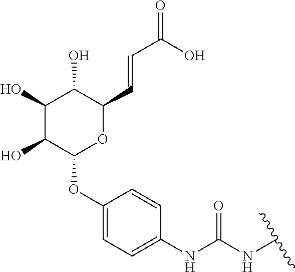 [SMILES: *NC(=O)Nc1ccc(O[C@H]2O[C@H](/C=C/C(=O)O)[C@@H](O)[C@H](O)[C@@H]2O)cc1] | 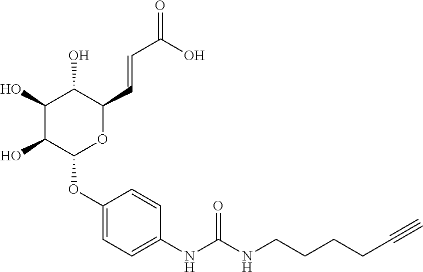 [SMILES: C#CCCCCNC(=O)Nc1ccc(O[C@H]2O[C@H](/C=C/C(=O)O)[C@@H](O)[C@H](O)[C@@H]2O)cc1] |
| X24 | 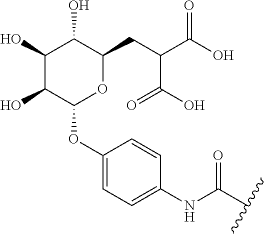 [SMILES: *C(=O)Nc1ccc(O[C@H]2O[C@H](CC(C(=O)O)C(=O)O)[C@@H](O)[C@H](O)[C@@H]2O)cc1] | 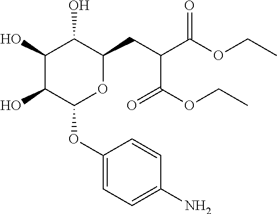 [SMILES: CCOC(=O)C(C[C@H]1O[C@H](Oc2ccc(N)cc2)[C@@H](O)[C@@H](O)[C@@H]1O)C(=O)OCC] |
| X25 | 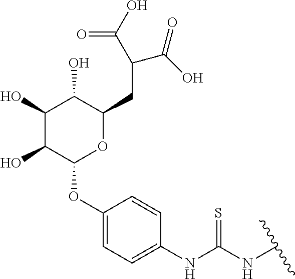 [SMILES: *NC(=S)Nc1ccc(O[C@H]2O[C@H](CC(C(=O)O)C(=O)O)[C@@H](O)[C@H](O)[C@@H]2O)cc1] | 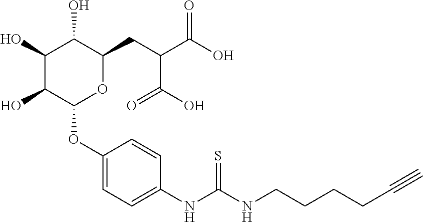 [SMILES: C#CCCCCNC(=S)Nc1ccc(O[C@H]2O[C@H](CC(C(=O)O)C(=O)O)[C@@H](O)[C@H](O)[C@@H]2O)cc1] |
| X26 | 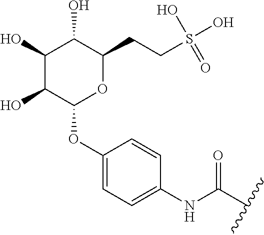 | 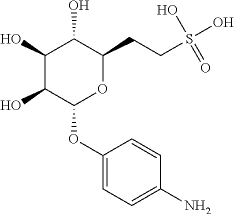 |
| X27 | 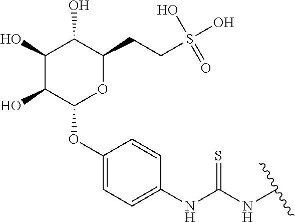 | 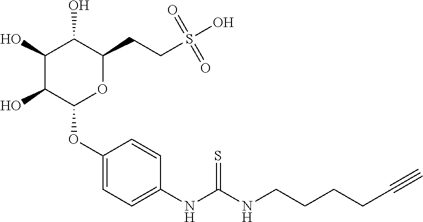 [SMILES: C#CCCCCNC(=S)Nc1ccc(O[C@H]2O[C@H](CCS(=O)(=O)O)[C@@H](O)[C@H](O)[C@@H]2O)cc1] |
| X28 | 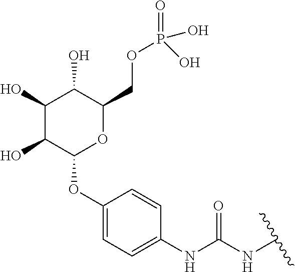 [SMILES: *NC(=O)Nc1ccc(O[C@H]2O[C@H](COP(=O)(O)O)[C@@H](O)[C@H](O)[C@@H]2O)cc1] | 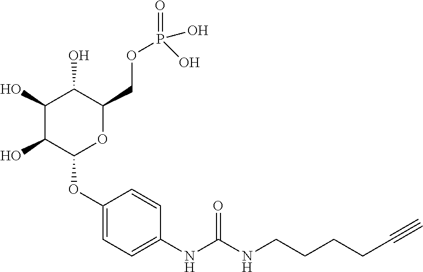 [SMILES: C#CCCCCNC(=O)Nc1ccc(O[C@H]2O[C@H](COP(=O)(O)O)[C@@H](O)[C@H](O)[C@@H]2O)cc1] |
| X29 | 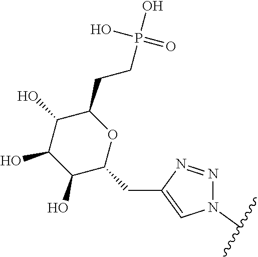 [SMILES: *n1cc(C[C@H]2O[C@H](CCP(=O)(O)O)[C@@H](O)[C@H](O)[C@@H]2O)nn1] | 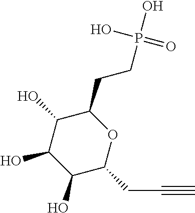 [SMILES: C#CC[C@H]1O[C@H](CCP(=O)(O)O)[C@@H](O)[C@H](O)[C@@H]1O] |
| X30 | 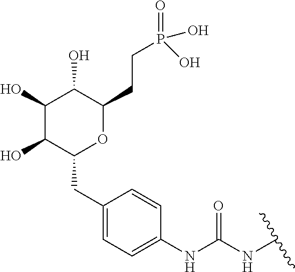 [SMILES: *NC(=O)Nc1ccc(C[C@H]2O[C@H](CCP(=O)(O)O)[C@@H](O)[C@H](O)[C@@H]2O)cc1] | 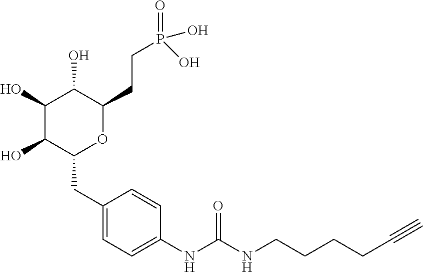 [SMILES: C#CCCCCNC(=O)Nc1ccc(C[C@H]2O[C@H](CCP(=O)(O)O)[C@@H](O)[C@H](O)[C@@H]2O)cc1] |
| X31 | 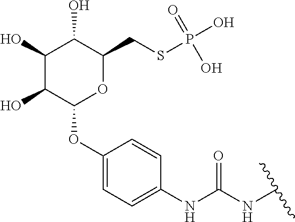 [SMILES: *NC(=O)Nc1ccc(O[C@H]2O[C@H](CSP(=O)(O)O)[C@@H](O)[C@H](O)[C@@H]2O)cc1] | 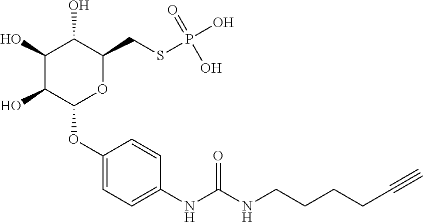 [SMILES: C#CCCCCNC(=O)Nc1ccc(O[C@H]2O[C@H](CSP(=O)(O)O)[C@@H](O)[C@H](O)[C@@H]2O)cc1] |
| X32 | 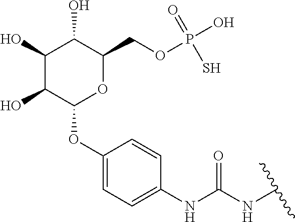 [SMILES: *NC(=O)Nc1ccc(O[C@H]2O[C@H](COP(=O)(O)S)[C@@H](O)[C@H](O)[C@@H]2O)cc1] | 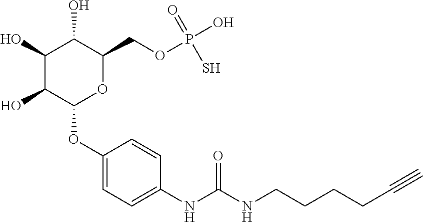 [SMILES: C#CCCCCNC(=O)Nc1ccc(O[C@H]2O[C@H](COP(=O)(O)S)[C@@H](O)[C@H](O)[C@@H]2O)cc1] |
| X32 | 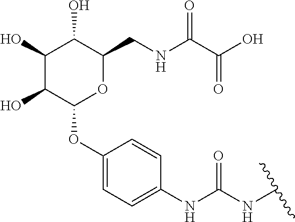 [SMILES: *NC(=O)Nc1ccc(O[C@H]2O[C@H](CNC(=O)C(=O)O)[C@@H](O)[C@H](O)[C@@H]2O)cc1] | 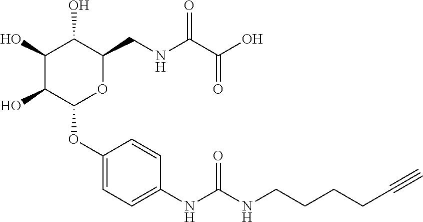 [SMILES: C#CCCCCNC(=O)Nc1ccc(O[C@H]2O[C@H](CNC(=O)C(=O)O)[C@@H](O)[C@H](O)[C@@H]2O)cc1] |
| X33 | 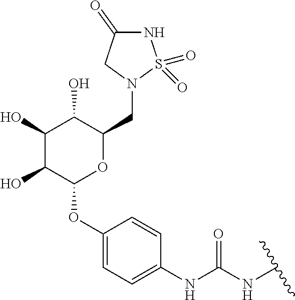 [SMILES: *NC(=O)Nc1ccc(O[C@H]2O[C@H](CN3CC(=O)NS3(=O)=O)[C@@H](O)[C@H](O)[C@@H]2O)cc1] | 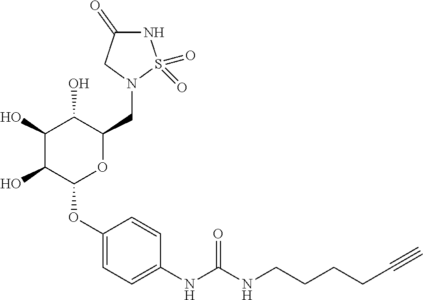 [SMILES: C#CCCCCNC(=O)Nc1ccc(O[C@H]2O[C@H](CN3CC(=O)NS3(=O)=O)[C@@H](O)[C@H](O)[C@@H]2O)cc1] |
| X34 | 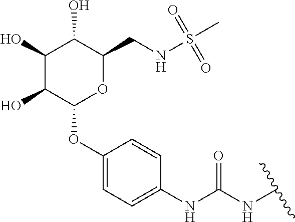 [SMILES: *NC(=O)Nc1ccc(O[C@H]2O[C@H](CNS(C)(=O)=O)[C@@H](O)[C@H](O)[C@@H]2O)cc1] | 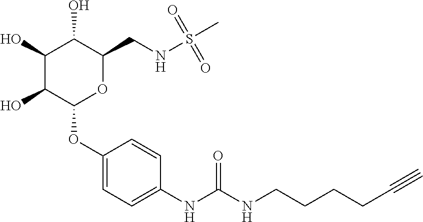 [SMILES: C#CCCCCNC(=O)Nc1ccc(O[C@H]2O[C@H](CNS(C)(=O)=O)[C@@H](O)[C@H](O)[C@@H]2O)cc1] |
| X35 | 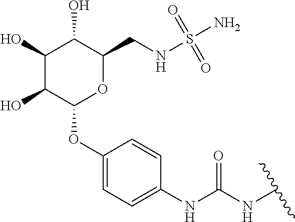 [SMILES: *NC(=O)Nc1ccc(O[C@H]2O[C@H](CNS(N)(=O)=O)[C@@H](O)[C@H](O)[C@@H]2O)cc1] | 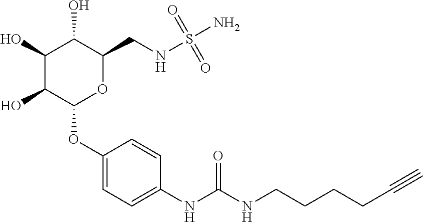 [SMILES: C#CCCCCNC(=O)Nc1ccc(O[C@H]2O[C@H](CNS(N)(=O)=O)[C@@H](O)[C@H](O)[C@@H]2O)cc1] |
| X36 | 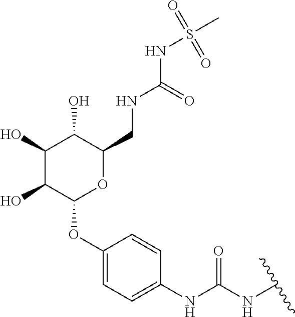 [SMILES: *NC(=O)Nc1ccc(O[C@H]2O[C@H](CNC(=O)NS(C)(=O)=O)[C@@H](O)[C@H](O)[C@@H]2O)cc1] | 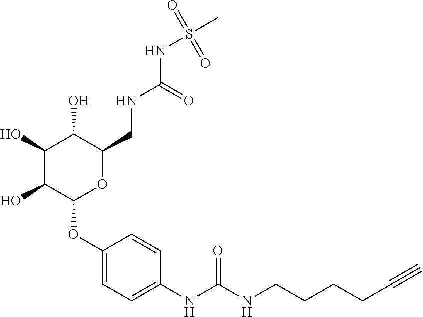 [SMILES: C#CCCCCNC(=O)Nc1ccc(O[C@H]2O[C@H](CNC(=O)NS(C)(=O)=O)[C@@H](O)[C@H](O)[C@@H]2O)cc1] |
| X37 | 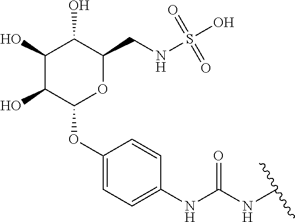 [SMILES: *NC(=O)Nc1ccc(O[C@H]2O[C@H](CNS(=O)(=O)O)[C@@H](O)[C@H](O)[C@@H]2O)cc1] | 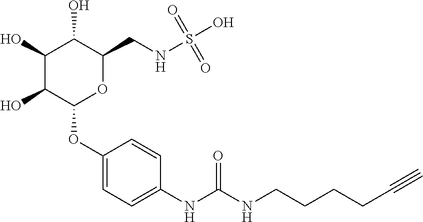 [SMILES: C#CCCCCNC(=O)Nc1ccc(O[C@H]2O[C@H](CNS(=O)(=O)O)[C@@H](O)[C@H](O)[C@@H]2O)cc1] |
| X38 | 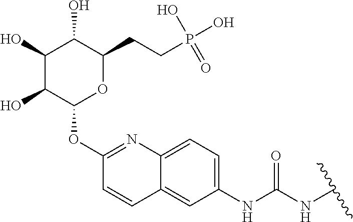 [SMILES: *NC(=O)Nc1ccc2nc(O[C@H]3O[C@H](CCP(=O)(O)O)[C@@H](O)[C@H](O)[C@@H]3O)ccc2c1] | 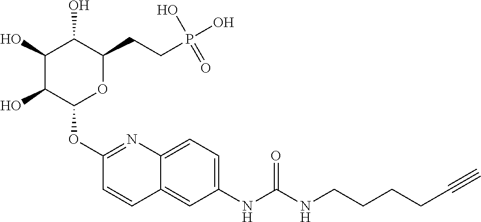 [SMILES: C#CCCCCNC(=O)Nc1ccc2nc(O[C@H]3O[C@H](CCP(=O)(O)O)[C@@H](O)[C@H](O)[C@@H]3O)ccc2c1] |

5.2.1.1.2 IGF-2 Polypeptides

In some embodiments, the cell surface receptor binding moiety comprises a polypeptide that binds a M6PR. In some embodiments, the cell surface receptor binding moiety comprises an insulin-like growth factor 2 (IGF-2) polypeptide sequence that binds CI-M6PR.

Insulin-like growth factor-2 (IGF-2) is a protein hormone encoded by the IGF2 gene and having growth-regulating, insulin-like and mitogenic activity. The terms “IGF-2 polypeptide”, “IGF-2 protein” and “IGF-2 peptide” are used interchangeably herein and refer to polypeptides that include an amino acid sequence corresponding to a naturally occurring IGF-2 protein hormone, variants thereof, truncated versions thereof, and/or a fragment thereof. In general, the IGF-2 polypeptides selected for incorporation into the bifunctional compounds or bridging composition of this disclosure are polypeptides capable of binding to a cell surface receptor and to trigger receptor-mediated endocytosis, thereby facilitating uptake of the viral composition into a lysosome in a cell.

Naturally occurring human IGF-2 hinds to a number of cell surface receptors with varying affinity, such as IGFR1, insulin receptor, and mannose-6-phosphate receptor (M6PR), IGF-2 can exert its biological effect primarily through interactions with the IGF1R and insulin receptor while interaction with the cation-independent M6P receptor (CI-M6PR) is believed to result in the IGF-2 being internalized to the lysosome where it is degraded.

A variety of IGF-2 polypeptides may be adapted for use in the bridging compositions of this disclosure. IGF-2 polypeptides of interest include, for example, Uniprot P01344. In certain embodiments, the amino acid sequence of the full-length, immature IGF2 sequence is used. In certain embodiments, a processed, mature IGF2 sequence is used.

In some embodiments, an IGF-2 polypeptide suitable for incorporation in the bifunctional bridging compositions of this disclosure is one that binds specifically to the CI-M6PR. Particularly useful are mutations, variations and/or truncations in the IGF-2 polypeptide that result in a variant polypeptide which binds the M6PR with a substantially equivalent or higher affinity, while binding other receptors of interest with reduced affinity, relative to a naturally occurring parental or wild type IGF-2 polypeptide. In some embodiments, the IGF-2 polypeptide is a variant IGF-2 polypeptide having enhanced affinity for the CI-M6PR as compared to naturally occurring human IGF-2 polypeptide.

In some embodiments, the IGF-2 polypeptide is a variant IGF-2 polypeptide that has diminished or decreased, or no affinity for the insulin receptor and/or IGF-1 receptor (IGF1R) as compared to a naturally occurring parental IG-F-2 polypeptide. In some embodiments, the IGF-2 peptide polypeptide is a variant having increased affinity for the M6PR as compared to a naturally occurring parental or wild type IGF-2 polypeptide. In some embodiments, the IGF-2 polypeptide has mutations or variations that result in a polypeptide which binds the M6PR with high affinity while no longer binding the other two receptors (insulin receptor and/or IGF1R) with appreciable affinity. In some embodiments, the IGF-2 polypeptide includes a substitution of residues Tyr 27 with Leu, Leu 43 with Val, and/or Ser 26 with Phe which diminishes the affinity of the resulting IGF-2 polypeptide for IGF1R (see e.g., Tones et al. (1995) J. Mol. Biol. 248(2)385-401).

In some embodiments, the IGF-2 polypeptide is a truncated polypeptide missing residues 1-7 of wild type IG-F-2 (e.g., mature human IG-F-2) which results in a relative decrease in affinity for the IGF1R (see e.g., Hashimoto et al. (1995) J. Biol. Chem. 270(30):18013-8). In some embodiments, the IGF-2 polypeptide is a truncated polypeptide missing residues 2-7 of wild type IGF-2 (e.g., mature human IGF-2). In some embodiments, the IGF-2 polypeptide is a C-terminal truncated polypeptide, e.g., a polypeptide missing the residues 62-67 of wild type IGF-2, which results in a lower affinity for the resulting IGF-2 polypeptide for IGF1R (see e.g., Roth et al. (1991) Biochem. Biophys. Res. Commun. 181(2):907-14).

In some embodiments, an IGF-2 polypeptide further contains a deletion or a replacement of amino acids corresponding to positions 2-7 of SEQ ID NO: 1. In some embodiments, an IGF-2 polypeptide further includes a deletion or a replacement of amino acids corresponding to positions 1-7 of SEQ ID NO:1. In some embodiments, an IGF-2 polypeptide further contains a deletion or a replacement of amino acids corresponding to positions 62-67 of SEQ ID NO:1. In some embodiments, an IGF-2 polypeptide further contains an amino acid substitution at a position corresponding to Tyr27, Leu43, or Ser26 of SEQ ID NO: 1. In some embodiments, an IGF-2 polypeptide contains at least an amino acid substitution selected from the group consisting of Tyr27Leu, Leu43Val, Ser26Phe and combinations thereof. In some embodiments, an IGF-2 polypeptide contains amino acids corresponding to positions 48-55 of SEQ ID NO:1. In some embodiments, an IGF-2 polypeptide contains at least three amino acids selected from the group consisting of amino acids corresponding to positions 8, 48, 49, 50, 54, and 55 of SEQ NO:1. In some embodiments, an IGF-2 polypeptide contains, at positions corresponding to positions 54 and 55 of SEQ ID NO:1, amino acids each of which is uncharged or negatively charged at pH 7.4. In some embodiments, the IGF-2 polypeptide has diminished binding affinity for the IGF-1 receptor (IGFR1) relative to the affinity of naturally occurring human IGF-2 for the IGF-1 receptor.

In some embodiments, the IGF-2 polypeptide is a variant IGF-2 polypeptide having diminished or no affinity for the insulin receptor and/or IGFR1 as compared to naturally occurring human polypeptide.

In some embodiments, the IGF-2 polypeptide is an active fragment of a wild type IGF-2 (e.g., mature human IGF-2). In some embodiments, the IGF-2 polypeptide is an active fragment having a sequence of 30 amino residues or less, such as 20 amino acid residues or less, 15 amino residues or less, 12 amino residues or less, or even 10 amino residues or less. In some embodiments, the IGF-2 polypeptide is an active fragment that includes residues 12-20, such as residues 12-20 of a wild type IGF-2 (e.g., mature human IGF-2), or a variant thereof. In some embodiments, the IGF-2 polypeptide is linked to the bifunctional compound via a spacer polypeptide (e.g., as described herein).

In some embodiments, the IGF-2 polypeptide is a variant modified to minimize binding to serum IGF-binding proteins (see e.g., Baxter (2000) Am. J. Physiol Endocrinol Metab, 278(6):967-76) to avoid sequestration of the bifunctional compounds in vivo. The IGF-2 polypeptide can be a variant where amino acid residues necessary for binding of IGF-2 polypeptides to IGF-binding serum proteins in vivo are replaced with variant residues that provide for reduced affinity for the IGF-binding serum proteins while retaining high affinity binding to M6PR, in some embodiments, the IGF-2 polypeptide is a variant including replacement of Phe-26 with Ser (see e.g., Bach et (1993) J. Biol. Chem. 268(13):9246-541), and/or replacement of Glu-9 with Lys.

Accordingly, the bifunctional bridging compositions of this disclosure can specifically bind to an internalizing M6PR cell surface receptor via binding of the IGF-2 polypeptide(s), In particular embodiments, the cell surface M6PR is a human 1146PR (e.g., human CI-M6PR).

In some embodiments, the variant IGF-2 polypeptide includes a replacement of Phe 26 of IGF-2 with Ser that provides for reduced affinity of the resulting variant IGF-2 polypeptide for serum IGFBP-1 and -6 with no effect on binding to the M6P/IGF-2 receptor. In some embodiments, the variant IGF-2 polypeptide includes other substitutions, such as Ser for Phe 19 and/or Lys for Glu 9.

In some embodiments, the bridging compositions or bifunctional compounds is a fusion of the IGF-2 polypeptide and a bridging moiety, e.g., a peptide, protein, or antibody or antibody fragment that specifically binds the viral composition, and the IGF-2 polypeptide is a IGF2 polypeptide variant that confers improved expression and/or secretion of a fusion protein bifunctional compound, compared to a naturally occurring IGF-2 polypeptide. In some embodiments, the IGF-2 polypeptide is a furin-resistant variant IGF-2 polypeptide having an amino acid sequence at least 70% identical to a IGF-2 polypeptide sequence of Table 2A, and a mutation that abolishes at least one furin protease cleavage site. Furin resistant IGF-2 polypeptides of interest include those described in U.S. Pat. No. 9,469,683.

In some embodiments, the IGF-2 polypeptide is a variant that includes amino acids 8-67 of mature human IGF-2 polypeptide. In some embodiments, the IGF-2 polypeptide is a variant that includes an Ala substitution at position Arg37 (e.g., SEQ ID NO: 6), where the IGF-2 polypeptide (i) has diminished binding affinity for the insulin receptor relative to the affinity of naturally-occurring human IGF-2 polypeptide for the insulin receptor, (ii) is resistant to furin cleavage and (iii) binds to the human cation-independent mannose-6-phosphate receptor in a mannose-6-phosphate-independent manner.

In some embodiments, the IGF-2 polypeptide is a variant that includes SEQ ID NO:3. In some embodiments, the IGF-2 polypeptide is a variant that includes one or more of the following modifications with respect to a parent IGF-2 sequence, of Table 2A:

substitution of arginine for glutamic acid at position 6;

deletion of amino acids 1-4 and 6;

deletion of amino acids 1-4, 6 and 7;

deletion of amino acids 1-4 and 6 and substitution of lysine for threonine at position 7;

deletion of amino acids 1-4 and substitution of glycine for glutamic acid at position 6 and substitution of lysine for threonine at position 7;

substitution of leucine for tyrosine at position 27; and

substitution of leucine for valine at position 43.

IGF-2 polypeptides of interest include those described in WO2005/078077, WO2012166653, WO2014/085621, U.S. Pat. Nos. 9,469,683, 10,301,369, 10,660,972 and WO2021/072372, the disclosures of which are incorporated herein by reference in their entirety. The sequences of exemplary IGF-2 polypeptides of interest are shown in Table 2A. In various embodiments, any one or more of the sequence variations, mutations and/or truncations described herein as imparting desirable properties on the IGF-2 polypeptides can be applied to a parental IGF-2 polypeptide sequence (e.g., a sequence of Table 2A) to produce a variant IGF-2 polypeptide of interest. All such variant IGF-2 polypeptide sequences are meant to be encompassed by this disclosure.

TABLE 2A
IGF-2 polypeptide sequences of interest

| SEQ ID | | |
|---|---|---|
| NO: | Name | Sequence |
| 1 | mature human | AYRPSETLCGGELVDTLQFVCGDRGFYFSRPASRVSRRSRGIV |
| | IGF-2 | EECCFRSCDLALLETYCATPAKSE |
| 2 | Δ2-7 IGF-2 | AALCGGELVDTLQFVCGDRGFYFSRPASRVSRRSRGIVEECCF |
| | | RSCDLALLETYCATPAKSE |
| 3 | Δ1-4 IGF-2 | SRTLCGGELVDTLQPVCGDRGFLFSRPASRVSRRSRGIVEECC |
| | | FRSCDLALLETYCATPARSE |
| 4 | 7-67 IGF-2 | TLCGGELVDTLQFVCGDRGFYFSRPASRVSRRSRGIVEECCFR |
| | | SCDLALLETYCATPAKSE |
| 5 | 8-67 IGF-2 | LCGGELVDTLQFVCGDRGFYFSRPASRVSRRSRGIVEECCFRS |
| | | CDLALLETYCATPAKSE |
| 6 | 8-67 IGF-2 | LCGGELVDTLQFVCGDRGFYFSRPASRVSARSRGIVEECCFRS |
| | (R37A) | CDLALLETYCATPAKSE |
| 7 | 12-21 IGF-2 | ELVDTLQFVS |
| | (C21S) (Tc | |
| | peptide) | |
| 8 | 12-21 IGF-2 | ELVDWLQFVS |
| | (T16W, C21S) (T1 | |
| | peptide) | |
| 9 | 12-21 IGF-2 | ELVDYLQFVS |
| | (T16Y, C21S) (T2 | |
| | peptide) | |
| 10 | 12-21 IGF-2 | ELVDTLQFVW |
| | (C21W) (T3 | |
| | peptide) | |
| 11 | 12-21 IGF-2 | ELVDFLQFVS |
| | (T16F, C21S) (T4 | |
| | peptide) | |
| 12 | 12-21 IGF-2 | ELVDTLQFVR |
| | (C21R) (T5 | |
| | peptide) | |
| 13 | (P431 peptide) | VHWDFRQWWOPS |

In some embodiments, the IGF-2 polypeptide has an amino acid sequence that is at least 80, 85, 90, 95, 96, 97, 98 or 99% identical to an IGF2 variant peptide of Table 2A. In some embodiments, the IGF-2 polypeptide comprises an amino acid sequence that is at least 90, 95, or 98% identical to an IGF2 variant peptide selected from SEQ ID NO: 1-6 of Table 2A. In some embodiments, the IGF-2 polypeptide comprises an amino acid sequence that is at least 80%, or 90% identical to an IGF2 variant peptide selected from SEQ ID NO: 7-13 of Table 2A.

In embodiments that utilize IGF-2 polypeptide sequences, the polypeptide may, for example, include the processed, immature IGF-2 polypeptide sequence (e.g., amino acids 1-91 of SEQ ID NO: 14, 15 or 16, the amino acid sequence of mature (without signal peptide) IGF-2 (e.g., amino acids 25-91 of SEQ ID NO: 14, 15 or 16), or any M6PR-binding portion of the IGF-2 polypeptide sequence. Representative IGF-2 polypeptide sequences that may be utilized are well known. See, e.g., Uniprot P01344.

Further Exemplary full-length IGF-2 polypeptide sequences (full-length, immature preprocessed) provided in 2B below.

TABLE 2B
Exemplary IGF-2 Polypeptide Sequences

| SEQ ID | | |
|---|---|---|
| NO. | Name | Sequence |
| 14 | Insulin-like | MGIPMGKSMLVLLTFLAFASCCIAAYRPSETLCGGELVDTLQFVCG |
| | growth factor | DRGFYFSRPASRVSRRSRGIVEECCFRSCDLALLETYCATPAKSERD |
| | II-Isoform 1 | VSTPPTVLPDNFPRYPVGKFFQYDTWKQSTQRLRRGLPALLRARR |
| | | GHVLAKELEAFREAKRHRPLIALPTQDPAHGGAPPEMASNRK |
| 15 | Insulin-like | MGIPMGKSMLVLLTFLAFASCCIAAYRPSETLCGGELVDTLQFVCG |
| | growth factor | DRGFYFRLPGRPASRVSRRSRGIVEECCFRSCDLALLETYCATPAKS |
| | II-Isoform 2 | ERDVSTPPTVLPDNFPRYPVGKFFQYDTWKQSTQRLRRGLPALLR |
| | | ARRGHVLAKELEAFREAKRHRPLIALPTQDPAHGGAPPEMASNRK |
| 16 | Insulin-like | MVSPDPQIIVVAPETELASMQVQRTEDGVTIIQIFWVGRKGELLRR |
| | growth factor | TPVSSAMQTPMGIPMGKSMLVLLTFLAFASCCIAAYRPSETLCGGE |
| | II-Isoform 3 | LVDTLQFVCGDRGFYFSRPASRVSRRSRGIVEECCFRSCDLALLET |
| | | YCATPAKSERDVSTPPTVLPDNFPRYPVGKFFQYDTWKQSTQRLR |
| | | RGLPALLRARRGHVLAKELEAFREAKRHRPLIALPTQDPAHGGAPP |
| | | EMASNRK |

5.2.1.2 ASGPR Binding Moieties

In some embodiments, the cell surface receptor targeted by the bifunctional bridging compositions of this disclosure is an asialoglycoprotein receptor.

The term “asialoglycoprotein receptor” (ASGPR), also known as the Ashwell Morell receptor, means the transmembrane glycoprotein receptor found primarily in hepatocytes which plays an important role in serum glycoprotein homeostasis by mediating the endocytosis and lysosomal degradation of glycoproteins with exposed terminal galactose or N-acetyl-galactosamine (GalNAc) residues. ASGPR cycles between endosomes and the cell surface. In particular embodiments, the ASGPR is Homo sapiens asialoglycoprotein receptor 1 (ASGR1) (see, e.g., NCBI Reference Sequence: NM_01197216).

In some embodiments of formula (Ia), X is a moiety that binds to a cell surface ASGPR. In some embodiments, the cell surface receptor binding moiety binds an ASGPR and comprises a GalNAc sugar moiety, or a variant thereof.

The ASGPR binding moiety (X) of the compounds and bifunctional bridging compositions of this disclosure can be a N-acetylgalactosamine (GalNAc), or an analog or derivative of GalNAc. A variety of ligands capable of binding ASGPR can be adapted for use in the bifunctional bridging compositions.

In certain embodiments of formula (Ia), each X is independently selected from the group consisting of formula (IIIj), formula (IIIk), formula (IIIl), and formula (IIIm):

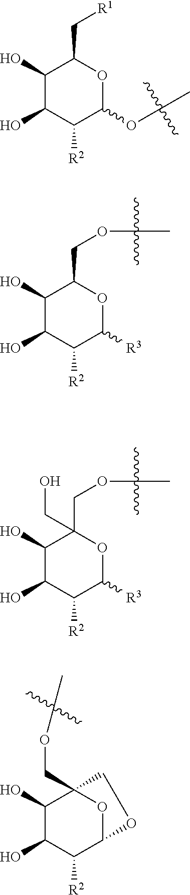

wherein R¹ is —OH, —OC(O)R, or

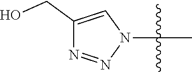

wherein R is C₁₋₆ alkyl;

wherein R² is selected from the group consisting of —NHCOCH₃, —NHCOCF₃, —NHCOCH₂CF₃, —OH, and

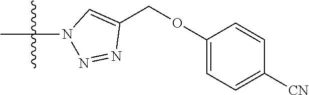 [SMILES: *n1cc(COc2ccc(C#N)cc2)nn1]

and

wherein R³ is selected from the group consisting of —H, —OH, —CH₃, —OCH₃, and —OCH₂CH═CH₂.

In certain embodiments, X is of formula (IIIo)

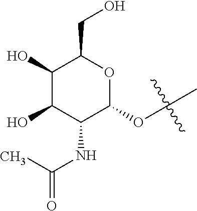 [SMILES: *O[C@H]1O[C@H](CO)[C@H](O)[C@H](O)[C@H]1NC(C)=O]

In certain embodiments, X is of formula:

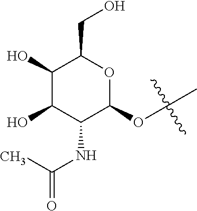 [SMILES: *O[C@@H]1O[C@H](CO)[C@H](O)[C@H](O)[C@H]1NC(C)=O]

In certain embodiments, X is of formula (IIIp)

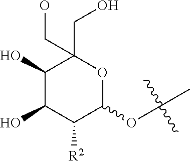

in certain embodiments, X is of formula (IIIo)

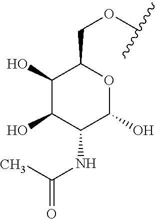 [SMILES: *OC[C@H]1O[C@H](O)[C@H](NC(C)=O)[C@@H](O)[C@H]1O]

In certain embodiments, X is of formula:

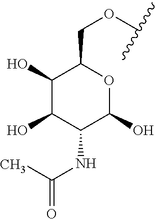 [SMILES: *OC[C@H]1O[C@@H](O)[C@H](NC(C)=O)[C@@H](O)[C@H]1O]

In certain embodiments, X is selected from the group consisting of formula (IIIj′), formula (IIIk′), formula (IIIl′), and formula (IIIm′):

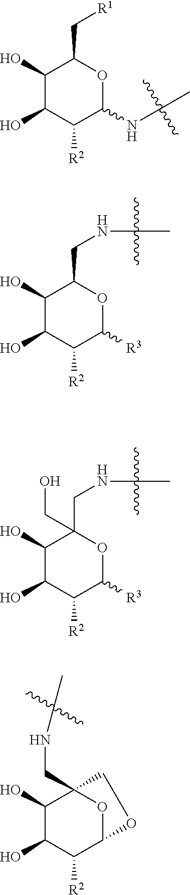

wherein R¹ is —OH, —OC(O)R,

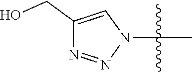 [SMILES: *n1cc(CO)nn1]

or wherein R is C₁₋₆ alkyl;

wherein R² is selected from the group consisting of —NHCOCH₃, —NHCOCF₃, —NHCOCH₂CF₃, —OH, and

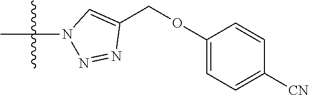 [SMILES: *n1cc(COc2ccc(C#N)cc2)nn1]

and

wherein R³ is selected from the group consisting of —H, —OH, —CH₃, —OCH₃, and —OCH₂CH═CH₂.

In certain embodiments, X is of formula (IIIo′)

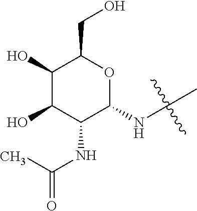 [SMILES: *N[C@H]1O[C@H](CO)[C@H](O)[C@H](O)[C@H]1NC(C)=O]

In certain embodiments, X is of formula (IIIp′)

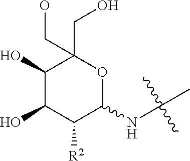

In certain embodiments of the compounds described herein, each X is independently selected from the group consisting of formulas (IIIc), (IIIb), (IIIc), (IIId), (IIIe), (IIIj), (IIIk), (IIIl), (IIIm), (IIIp), (IIIj′), (IIIk′), (IIIl′), (IIIm′), and (IIIp′).

In one embodiment, the compound of formula (Ia) is selected from the compounds of Table 11. In one embodiment, the compound of formula (Ia) is selected from the compounds of Table 12.

Exemplary ASGPR binding compounds of formula (Ia) are shown in Tables 1142.

5.2.1.3 Folate Receptor Binding Moieties

In some embodiments, the cell surface receptor is targeted by the bifunctional bridging compositions of this disclosure is folate receptor.

The term “folate receptor” or “FR” refers to a class of transmembrane glycoprotein receptors, of which there are four members, FRα, FRβ, FRγ and FRδ. Folate receptors can be overexpressed on a vast majority of cancer tissues. The folate receptor binds folate and folic acid derivatives and transports them into the cell by receptor-mediated endocytosis. The folate receptor cycles between endosomes and the cell surface. In particular embodiments, the folate, receptor is Homo sapiens folate receptor alpha (FOLR1) (see, e.g., NCBI Reference Sequence: NM_000802). In particular embodiments, the folate receptor is Homo sapiens folate receptor beta (FOLR2) (see, e.g., NCBI Reference Sequence: NM_000803).

In some embodiments, the folate receptor folate receptor 1 (FRα), or 2 (FRβ). In some embodiments, the cell surface receptor binding moiety binds a folate receptor, e.g., a folate receptor 1 (FRα), or folate receptor 2 (FRβ) and comprises a folic acid moiety or an small molecule anti-folate moiety.

In some embodiments, the folate binding moiety X includes a folate heterocyclic ring, or analog thereof, that is linked via a linking moiety comprising a cyclic group (e.g., aryl, heteroaryl, heterocycle, or cycloalkyl) to an amino acid derivative (e.g., a glutamic acid). The linking moiety can be of 1-10 atoms in length, such as 1-6, or 1-5 atoms in length. The linking moieties cyclic group can be any convenient group including, aryl, (e.g. phenyl), heteroaryl, (e.g., pyridine, thiophene), heterocyclic (e.g., piperidine), cycloalkyl (e.g., cyclohexyl), and bicycloalkyl groups. In some embodiments, the linking moieties cyclic group is aryl. The amino acid derivative can be any convenient amino acid group including, gigantic acid, and aspartic acid.

In some embodiments, the folate heterocyclic ring of X is linked via an optionally substituted aryl or heteroaryl group to an amino acid derivative (e.g., a glutamic acid) that together provide a moiety having a desirable binding affinity and activity at the folate receptor of interest. Multiple folate binding moieties X can be linked together to provide multivalent binding to the folate receptor. The folate binding moiety or moieties X can be further linked to any convenient moiety or molecule of interest (e.g., as described herein). In certain embodiments, the folate binding moiety X includes a glutamic acid moiety that is linked to a molecule of interest via a linker. In certain cases, the folate binding moiety X is linked to the molecule of interest via a linker covalently bonded to the gamma position of the glutamic acid moiety. In other cases, the folate binding moiety X is linked to the molecule of interest via a linker covalently bonded to the alpha position of the glutamic acid moiety.

In some embodiments, the bifunctional bridging compositions of this disclosure (e.g., of formula (Ia)) include a binding moiety for a folate receptor that is of formula (Ic):

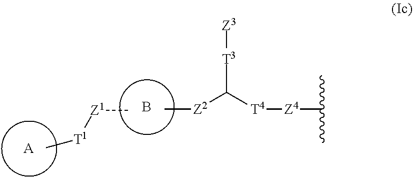

wherein:

A is a ring system of formula (II):

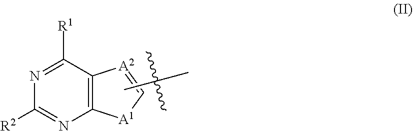

or a tautomer thereof, wherein:

R¹ and R² are independently selected from OH, NR²¹, and optionally substituted (C₁-C₆)alkyl (e.g., —CH₃ or —CH₂OH);

A¹ is selected from —N═CR³—, —CR³═N—, —CR³═CR³—, NR²¹, S, O, and C(R⁴)₂;

A² is selected from N, and CR³;

each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and

each R⁴ is independently selected from H, halogen (e.g., F), and optionally substituted (C₁-C₆)alkyl

T¹ is an optionally substituted (C₁-C₃)alkylene;

Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;

B is a ring system selected from optionally substituted aryl, optionally substituted heteroaryl, optionally substituted heterocycle, optionally substituted cycloalkyl, and optionally substituted bridged bicycle;

Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;

Z³ is carboxyl or carboxyl bioisostere, or a prodrug thereof;

T³ is absent, or is selected from optionally substituted (C₁-C₆)alkylene;

Z⁴ is optionally substituted (C₁-C₆)alkylene (e.g., —CH₂CH₂—), or is absent;

Z⁴ is a linking moiety (e.g., a linking moiety selected from ester, amide, urea, thiourea, sulfonamide, amine, ether, optionally substituted aryl, optionally substituted heterocycle, and optionally substituted heteroaryl);

each R²¹ is independently selected from H, and optionally substituted (C₁-C₆)alkyl; and [symbol] represents the point of attachment to -L-Y (e.g., as described herein).

The folate binding moiety X of formula (Ic) can be incorporated into the bifunctional bridging compositions of this disclosure by attachment of a bridging moiety (Y) to the Z⁴ group via a linking moiety, it is understood that in the bifunctional bridging compositions of formula (Ia) and (Ic), the group or linking moiety attached to Z⁴ can, in some cases, be considered to be part of the folate binding moiety (X) and provide for desirable binding to the folate receptor. In certain other cases, the group or linking moiety attached to Z⁴ can be considered part of the linker L (e.g., of formula (IV) as described herein).

Accordingly, in one embodiment of formula (Ia), provided herein are folate binding compounds of formula (Id):

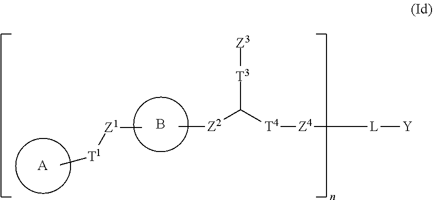

or a salt thereof,
wherein:

T¹ is an optionally substituted (C₁-C₃)alkylene;

Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;

B is a ring system selected from optionally substituted aryl, optionally substituted heteroaryl, optionally substituted heterocycle, optionally substituted cycloalkyl, and optionally substituted bridged bicycle;

Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;

Z³ is carboxyl or carboxyl bioisostere, or a prodrug thereof;

T³ is absent, or is selected from optionally substituted (C₁-C₆)alkylene;

T⁴ is optionally substituted (C₁-C₆)alkylene (e.g., —CH₂CH₂—), or is absent.

Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);

each R²¹ is independently selected from H. and optionally substituted (C₁-C₆)alkyl;

n is 1 to 100:

L is a linker;

Y is a moiety of interest; and

A is a ring system of formula (II):

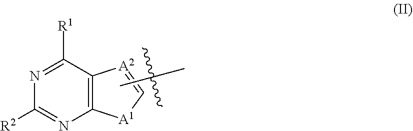

or a tautomer thereof, wherein:

R¹ and R² are independently selected from OH, NR²¹, and optionally substituted (C₁-C₆)alkyl (e.g., —CH₃ or —CH₂OH);

A¹ is selected from —N═CR³—, —CR³═N—, —CR³═CR³—, NR²¹, S, O, and C(R⁴)₂;

A² is selected from N, and CR³;

each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and

each R⁴ is independently selected from H, halogen (e.g., F), and optionally substituted (C₁-C₆)alkyl.

In some embodiments of formula (Id), at least one of following applies:

1) T³ is optionally substituted (C₁-C₆)alkylene (e.g., —CH₂CH₂—);
2) L is a non-cleavable linker;
3) when A is of formula (II-A) or (II-A′), or a tautomer thereof:

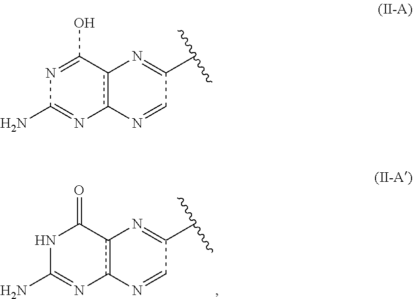

then Z¹ is not NR²¹, and/or B is not 1,4-linked phenyl;

4) when A is of formula (II-B), or a tautomer thereof:

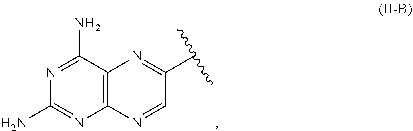 [SMILES: *c1cnc2nc(N)nc(N)c2n1]

then Z¹ is not NR²¹, and/or B is not 1,4-linked phenyl; and/or

5) when A is of formula (II-C) or (II-C′), or a tautomer thereof:

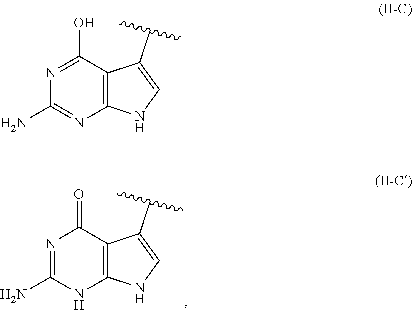 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

In some embodiments of formula (Id), T³ is optionally substituted (C₁-C₆)alkylene. In certain cases, T³ is (C₁-C₆)alkylene, i.e., hexyl, pentyl, butyl, propyl, ethyl or methyl. In certain cases, T³ is (C₁-C₃)alkylene. In certain cases, T³ is —CH₂CH₂CH₂—. In certain cases, T³ is —CH₂CH₂—. In certain cases, T³ is —CH₂—.

In some embodiments of formula (Id), V is absent. Accordingly, in some embodiments, the bifunctional bridging composition is of formula (IIIA):

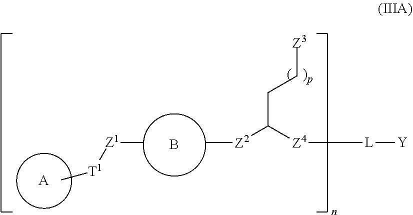

wherein p is 0 or 1.

In certain other embodiments of formula (Id), T⁴ is optionally substituted (C₁-C₆)alkylene. In certain cases, each T⁴ is (C₁-C₆)alkylene, i.e., hexyl, pentyl, butyl, propyl, ethyl or methyl. In certain cases, each T⁴ is (C₁-C₃)alkylene. In certain cases, each T⁴ is —CH₂CH₂CH₂—. In certain cases, each T⁴ is —CH₂CH₂—. In certain cases, each T is —CH₂—.

In some embodiments of formula (Id), T³ is absent. Accordingly, in some embodiments, the bifunctional bridging composition is of formula (IIIB):

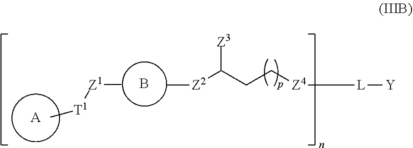

wherein p is 0 or 1.

In certain embodiments of any one of formulae (Id), (IIIA) or (IIIB), Z³ is a carboxyl group, or a prodrug thereof. In certain other embodiments. Z³ is a carboxyl bioisostere, or a prodrug thereof. A carboxyl bioisostere is a group with similar physical or chemical properties to a carboxyl group. In certain cases, the carboxyl bioisostere produces broadly similar biological properties to the corresponding carboxyl group. In certain cases, the carboxyl bioisostere may modify the activity of the compound, and may alter the metabolism of the compound. The subject compounds can include both acyclic and cyclic carboxylic acid bioisosteres. Carboxyl bioisosteres that can be utilized in the subject compounds includes, but is not limited to, hydroxamic acids, phosphonic acids, sulphonic acids, sulfonamides, acylsulfonamides, sulfonylureas, tetrazoles, thiazolidinediones, oxazolidinediones, 5-oxo-1,2,4-oxadiazole, 5-oxo-1,2,4-thiadiazole, 5-thioxo-1,2,4-oxadiazole, isothiazoles, difluorophenols, tetramic acids, squaric acids, 3-hydroxyquinolin-2-ones, and 4-hydroxyquinolin-2-ones. In certain embodiments, the carboxyl bioisostere is a moiety as described in Ballatore et al. 2013, Chem Med Chem., 8(3): 385-395.

In certain embodiments, a prodrug derivative of the carboxyl bioisostere group (Z³) may be incorporated into the compounds. For example, an ester prodrug group (e.g., —CO₂Et, or —CO₂CH₂CH₂—R″, where R″ is a heterocycle, e.g., N-morpholino) is included instead of a carboxylic acid group.

The term “pro-drug” refers to an agent which is converted into the active moiety in vivo by some physiological chemical process (e.g., a prodrug on being brought to the physiological pH is converted to the desired form).

In certain embodiments, the carboxyl bioisostere, or a prodrug thereof, is a moiety of one of the following structures:

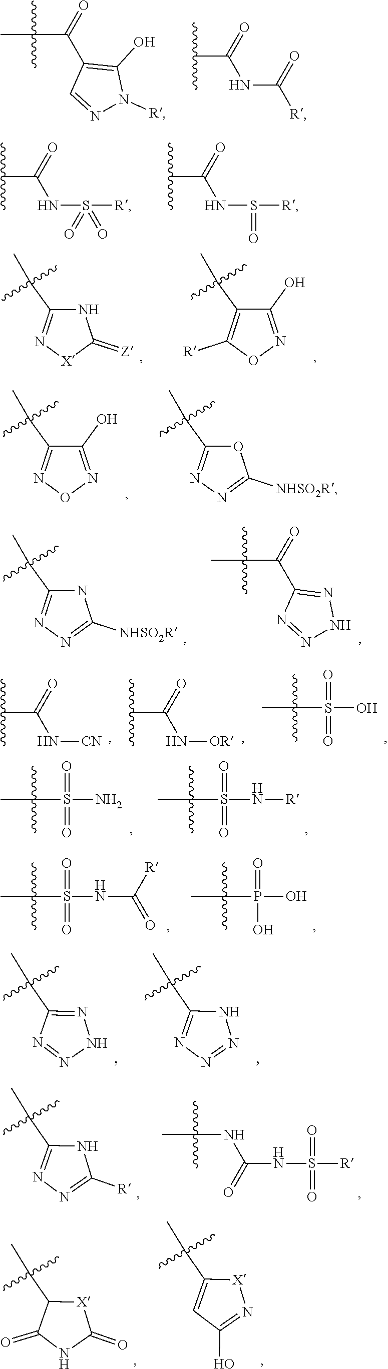

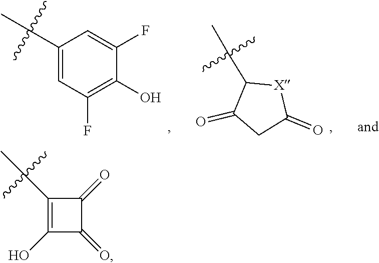

where:
each R′ is independently H or an optionally substituted moiety selected from (C₁₋₁₀)alkyl, (C₂₋₁₀)alkenyl, (C₂₋₁₀)heteroalkyl, (C₃₋₈)cyclic ring selected from cycloalkyl, aryl, heterocycle, or heteroaryl;
each X′ is independently O or S; and

### X″ is NH, O, or CH₂.

In certain embodiments, Z³ is selected from —COOH, —COOR²², —CH₂OH, —CH₂OR²², —CN, and tetrazole, wherein R²² is optionally substituted (C₁-C₆)alkyl. In certain cases, Z³ is —COOH. In certain cases, Z³ is —COOR²², and R²² is optionally substituted (C₁₋₃)alkyl. In certain cases, R²² is methyl, ethyl or propyl. In certain cases, R²² is substituted methyl, ethyl, or propyl. In certain cases, Z³ is —CH₂OH, or —CH₂OR²², and R²² is optionally substituted (C₁₋₃)alkyl. In certain cases, Z³ is —CN. In certain cases, Z³ is tetrazole.

In certain embodiments, Z³ is selected from one of the following structures:

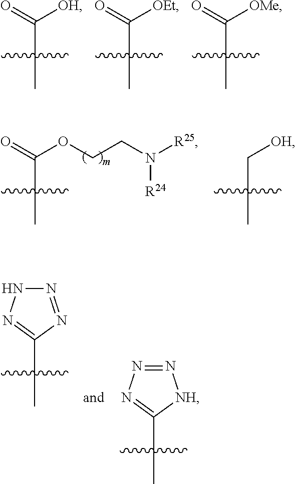

wherein:

R²⁴ and R²⁵ are independently selected from H and optionally substituted (C₁-C₆)alkyl, or R²⁴ and R²⁵ are cyclically linked to provide an optionally substituted 5 or 6-membered heterocycle; and

m is 1 to 5. In certain cases, R²⁴ and R²⁵ are H. In certain embodiments, RN and R^{2S} is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, Z³ is of the following structure:

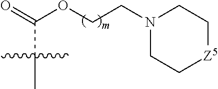

wherein

Z⁵ is O, NH or NR²¹; and

R²¹ is (C₁-C₆)alkyl.

In certain cases, Z⁵ is 0 and m is 1. In certain cases, Z⁵ is NH, and m is 1. In certain cases, Z⁵ is NCH₃, and m is 1.

In some embodiments of any one of formulae (Id), (IIIA) or (IIIB), Z² is a linking moiety (e.g., as described herein). In certain cases, Z² is an optionally substituted amide. In certain cases, Z² is an optionally substituted sulfonamide. In certain cases, Z² is an optionally substituted urea. In certain cases, Z² is an optionally substituted thiourea. In certain embodiments, Z² is —CONR²¹—. In certain cases, Z² is —O—. In certain case, Z² is —S—. In certain cases, Z² is an optionally substituted (C₁-C₆)alkylene. In certain cases, Z² is an amide bioisostere (e.g., as described herein below).

In certain embodiments, Z² is —CONR²¹—, wherein R²¹ is selected from H, and optionally substituted (C₁-C₆)alkyl. In certain cases, R²¹ is H. In certain other cases, R²¹ is optionally substituted (C₁-C₃)alkyl. In certain cases, R²¹ is methyl. In certain cases, R²¹ is ethyl.

In certain embodiments, Z² is —CONR²¹—, —SO₂NR²¹—, —NR²¹CO—, —NR²¹C(═O)NR²¹—, or —NR²¹C(═S)NR²¹, wherein each R²¹ is independently selected from H, and optionally substituted (C₁-C₆)alkyl. In certain cases, each R²¹ is H. In certain other cases, each R²¹ is optionally substituted (C₁-C₃)alkyl. In certain cases, R²¹ is methyl. In certain cases, R²¹ is ethyl.

In certain cases of any one of formulae (Id), (IIIA) or (IIIB), Z⁴ is a linking moiety selected from ester, amide, sulfonamide, urea, thiourea, amine, ether, thioether, optionally substituted aryl, optionally substituted heterocycle, and optionally substituted heteroaryl. In certain cases, Z⁴ is a linking moiety selected from amide or amide bioisostere. In certain cases, Z⁴ is an amine. In certain cases, Z⁴ is an ether. In certain cases, Z⁴ is a thioether. In certain cases, Z⁴ is an optionally substituted aryl. In certain cases, Z⁴ is a 1,4-phenyl group. In certain cases, Z⁴ is an optionally substituted heteroaryl. In certain cases, Z⁴ is a oxadiazole. In certain cases, Z⁴ is a triazole.

In certain cases, Z′ is an amide bioisotere. An amide bioisostere is a group with similar physical or chemical properties to an amide group. In certain cases, the amide bioisostere produces broadly similar biological properties to the corresponding amide group, in certain cases, the amide bioisostere may modify the activity of the compound, and may alter the metabolism of the compound. The subject compounds can include both acyclic and cyclic amide bioisosteres. Amide bioisosteres that can be utilized in the subject compounds includes, but is not limited to, imidazoles, triazoles, thiazoles, oxadiazoles, tetrazoles, indoles, olefins, fluoroalkenes, ureas, esters, thioamides, phosphonamidates, sulfonamides, trifluoro ethylamines, amidines, and carbamates. In some cases, the amide bioisotere is a 5-membered ring heterocycle, e.g., a triazole, an oxadiazole, an imidazole, a tetrazole, or a pyrazole. In certain cases, the amide bioisostere is a six membered heteroaryl, e.g., a pyrazine or a pyridine. In certain cases, the amide bioisostere is a retroinverted, or reverse amide, e.g., —NHC(O)— converted to —C(O)NH—. In certain cases, the amide bioisostere is a urea. In certain cases, the amide bioisostere is a carbamate, in certain cases, the amide bioisostere is an amidine. In certain cases, the amide bioisostere is a thioamide, in certain cases, the amide bioisostere is a trifluoroethylamine. In certain cases, the amide bioisotere is a sulfonamide. In certain cases, the amide bioisostere is a phosphonamidate. In certain cases, the amide bioisostere is an olefin. In certain embodiments, the amide bioisotere is a moiety as described in Kumari et al. 2020, J. Med. Chem., 63: 12290-123.58. In certain embodiments, the amide bioisostere is a Mole ty of one of the following structures:

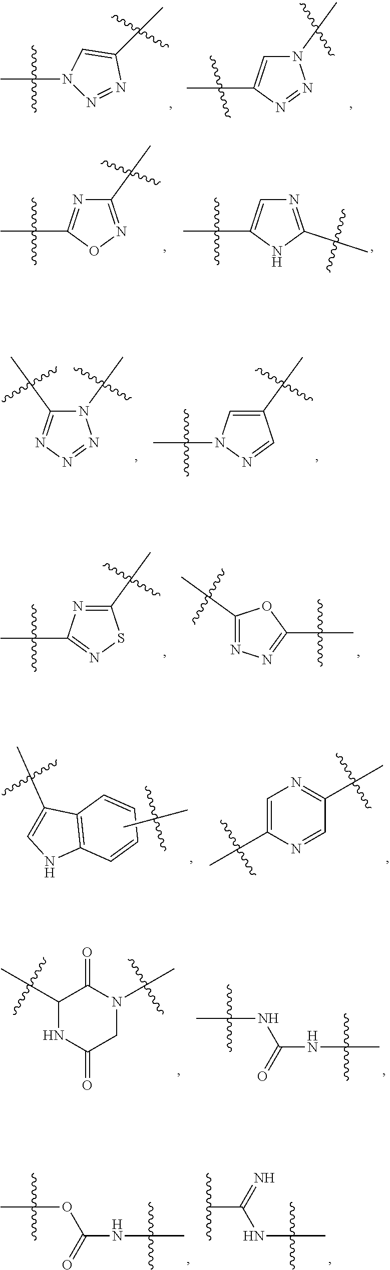

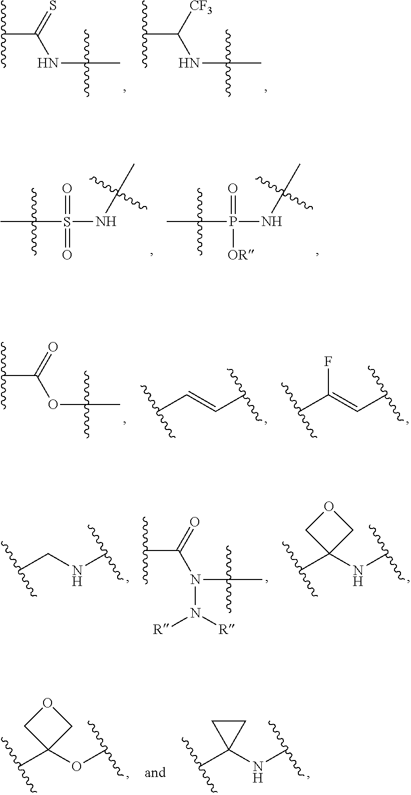

where R″ is an optionally substituted (C₁-C₆)alkyl.

In certain cases, Z⁴ is a linking moiety selected from —CONR²¹—, —O—, —S—, optionally substituted aryl (e.g., 1,4-phenyl) and optionally substituted heteroaryl (e.g., oxadiazole or triazole), wherein R²¹ is selected from H, and optionally substituted (C₁-C₆)alkyl. In certain cases, R²¹ is methyl. In certain cases, R²¹ is ethyl.

In some embodiments, Z⁴ is a linking group selected from:

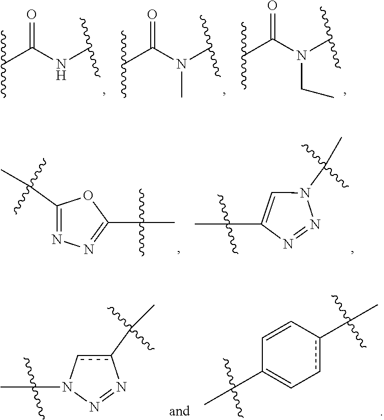

In some embodiments of formula (Id) or (IIIA), —Z²CH(-T³-Z³)T⁴Z⁴— is selected from the following structures:

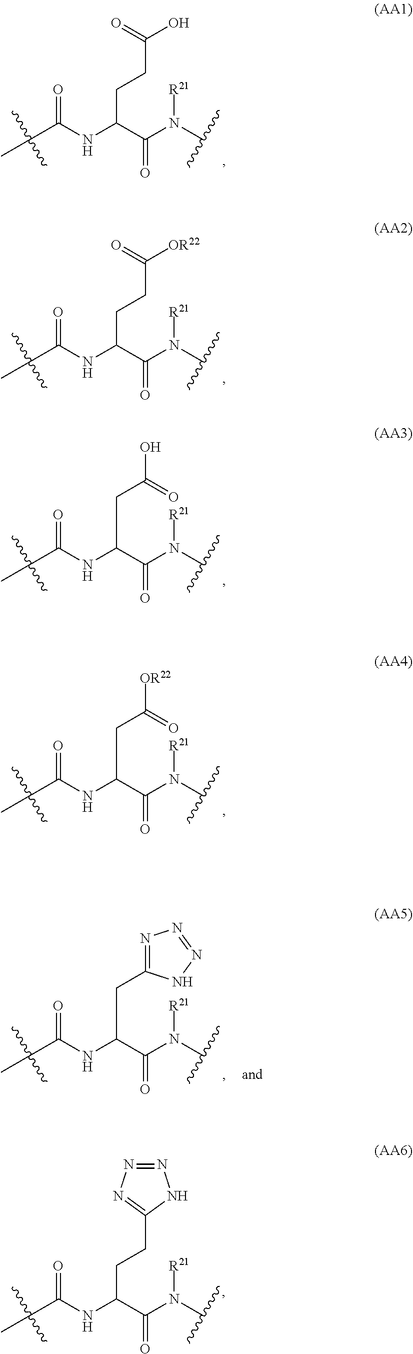

or a tautomer thereof, or a salt thereof.

In some embodiments of form a (Id) or (RIB), —Z²CH(-T³-Z³)T⁴Z⁴— of formula (I) is selected from the following structures:

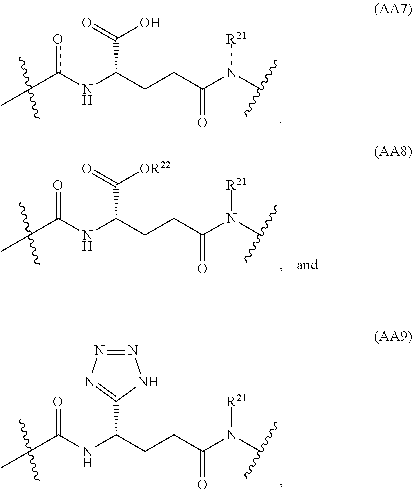

or a tautomer thereof, or a salt thereof.

In certain cases of (AA2), (AA4) or (AA8), R²² is optionally substituted (C₁-C₆)alkyl. In certain cases, R²² is methyl. In certain cases, R²² is ethyl. In some cases, R²² is propyl. In certain cases, R²² is substituted (C₁-C₆)alkyl. In certain cases, R²² is of the formula —(CH₂)mCH₂N(R²⁴)(R²⁵), where R²⁴ and R²⁵ are independently selected from H and optionally substituted (C₁-C₆)alkyl, or R²⁴ and R²⁵ are cyclically linked to provide an optionally substituted 5 or 6-membered heterocycle, and m is 1 to 5. In certain cases, R²⁴ and R²⁵ are H. In certain embodiments, R²⁴ and R²⁵ is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, R²² is of the following structure:

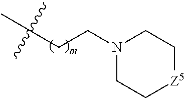

wherein

Z⁵ is O, NH or NR²¹; and R²¹ is (C₁-C₆)alkyl. In certain cases, Z⁵ is O and m is 1. In certain cases, Z⁵ is NH, and m is 1. In certain cases, Z⁵ is NCH₃, and m is 1.

In certain embodiments of any one of (AA1)-(AA9), R²¹ is H, In certain cases, R²¹ is methyl. In certain cases, R²¹ is ethyl. In certain cases, R²¹ is propyl. In certain cases, R²¹ is propargyl.

In some embodiments of formula (Id) or (IIIA), —Z²CH(-T³-Z³)T⁴Z⁴— is of the structure (AA1). In certain cases, —Z²CH(-T³-Z³)T⁴Z⁴— is of the structure (AA2). In certain cases, —Z²CH(-T³-Z³)T⁴Z⁴— is of the structure (AA3). In certain cases, —Z²CH(-T³-Z³)T⁴Z⁴— is of the structure (AA4). In certain cases, —Z²CH(-T³-Z³)T⁴Z⁴— is of the structure (AA5). In certain cases, —Z²CH(-T³-Z³)T⁴Z⁴— is of the structure (AA6).

In certain embodiments of formula (Id) or (IIIB), —Z²CH(-T³-Z³)T⁴Z⁴— is of the structure (AA7). In certain cases, —Z²CH(-T³-Z³)T⁴Z⁴— is of the structure (AA8). In certain other cases, —Z²CH(-T³-Z³)T⁴Z⁴— is of the structure (AA9).

In certain embodiments of the subject compounds, A¹ of ring system A is selected from —N═CR³—, —CR³═N—, or —CR³═CR³—. In certain cases, A¹ of ring system A is N═CR³—. In certain cases, A¹ of ring system A is —CR³═N—. In certain other cases, A¹ of ring system A is —CR³═CR³—.

In some embodiments of the subject bifunctional bridging compositions, A is of formula (IIA):

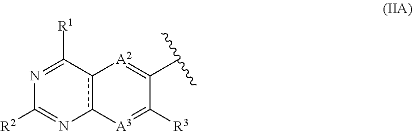

or a tautomer thereof, or a salt thereof, wherein:

A² is selected from N, and CR³;

A³ is independently selected from N, and CR²¹.

In certain embodiments of formula (IIA), A² and A³ are each N. In certain embodiments, A² is N and A³ is CR²¹. In certain cases, A² is CR³ and A³ is N. In certain other embodiments, A² and A³ are each independently CR³.

In certain embodiments of formula (IIA), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR²¹ or —COOR²¹. In certain other cases, R³ is —CONHR²¹, or —NHCOR²¹.

In certain embodiments of formula (IIA), R² is —NH₂. In certain embodiments. R² is optionally substituted (C₁-C₆)alkyl. In certain embodiments, R² is —CH₃. In certain embodiments, R² is —CH₂OH. In certain other embodiments, R² is H.

In certain embodiments of formula (IIA), R¹ is OH. In certain embodiments, R² is NH₂.

In certain embodiments of the subject bifunctional bridging composition, A is selected from:

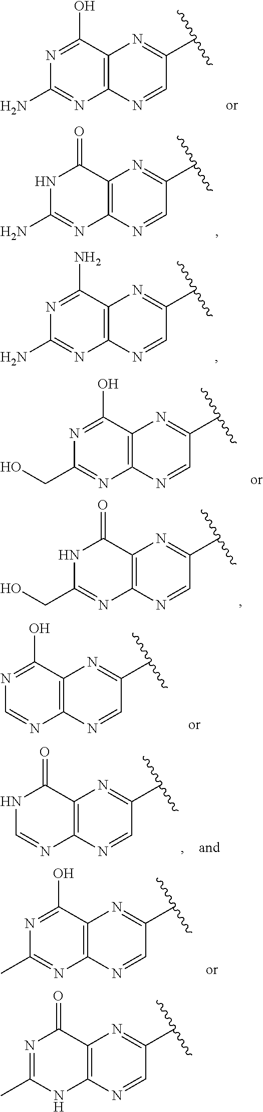

or a tautomer thereof.

In certain embodiments of the subject bifunctional bridging composition, A¹ of ring system A is selected from —NR²¹—, —S—, —O— or —C(R²¹)₂—. In certain cases, A¹ of ring system A is —NR^{<}—. In certain cases, A¹ of ring system A is —S—. In certain cases, A¹ of the ring system A is —O—. In certain other cases. A¹ of ring system A is —C(R²¹)₂—.

In some embodiments of the subject bifunctional bridging composition, A is of formula (IIB) or (IIC):

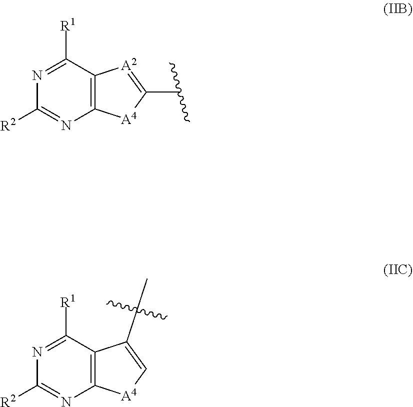

or a tautomer thereof, or a salt thereof, wherein A⁴ is selected from NR²¹, S, and O.

In certain embodiments of formula (IIB). A² is CR³. In certain cases, A² is N. In certain cases of formula (IIB), A⁴ is NR²¹. In certain cases, A⁴ is S. In certain other embodiments. A⁴ is O. In certain embodiments, A² is CR³ and A⁴ is NR²¹.

In certain embodiments of formula (IIB), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR² or —COOR². In certain other cases, R³ is —CONHR²¹, or —NHCOR²¹.

In certain embodiments of formula (IIB), R² is —NH₂. In certain embodiments, R² is optionally substituted (C₁-C₆)alkyl. In certain embodiments, R² is —CH₃. In certain embodiments, R² is —CH₂OH. In certain other embodiments, R² is H.

In certain embodiments of formula (IIB), R¹ is OH. In certain embodiments, R² is NH₂.

In certain embodiments of the subject bifunctional bridging compositions, A is selected from:

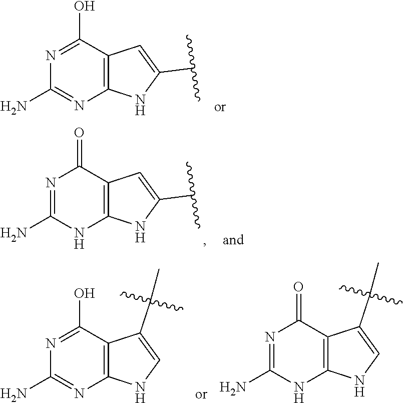

or a tautomer thereof.

In certain embodiments of any one of formulae (Id), (IIIA) or (IIIB), T¹ is CH₂. In certain embodiments, T¹ is CH₂CH₂. In certain other embodiments, T¹ is CH₂CH₂CH₂.

In certain embodiments of any one of formulae (Id), (IIIA) or (IIIB), Z¹ is NR²¹. In certain cases, R²¹ is H. In certain cases, R²¹ is methyl. In certain cases, R²¹ is ethyl. In certain cases, R²¹ is propyl. In certain cases, R²¹ is propargyl.

In certain cases of any one of formulae (Id), (IIIA) or (IIIB), Z¹ is O. In certain other cases. Z¹ is S.

In certain cases of any one of formulae (Id), (IIIA) or (IIIB), Z¹ is substituted methylene. In certain cases of any one of formulae (Id), (IIIA) or (IIIB), Z¹ is methylene substituted with propargyl (i.e., —CH(propargyl)-. In certain cases of any one of formulae (Id), (IIIA) or (IIIB), Z¹ is methylene substituted with (C₁-C₃)alkyl.

In certain embodiments of any one of formulae (Id), (IIIA) or (IIIB), T¹-Z¹ is optionally substituted (C₁-C₆)alkylene. In certain cases, T¹-Z¹ is —CH₂CH₂—. In certain cases, T¹-Z¹ is —CH₂CH₂CH₂CH₂—. In certain cases, T¹-Z¹ is —CH₂CH₂CH₂—. In certain embodiments of any one of formulae (Id), (IIIA) or (IIIB), T¹-Z¹ is —CH₂CH(propargyl)-.

In certain embodiments of the subject bifunctional bridging composition, the B ring system is an optionally substituted aryl. In certain cases, the B ring system is an optionally substituted heteroaryl. In certain cases, the B ring system is an optionally substituted heterocycle. In certain cases, the B ring system is an optionally substituted cycloalkyl. In certain other cases, the B ring system is an optionally substituted bridged bicycle.

In certain embodiments of the subject bifunctional bridging composition, the B ring system is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl (e.g., bicyclo[1.1.1]pentane).

In certain embodiments of the subject bifunctional bridging composition, the B ring system is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

In certain embodiments of the subject bifunctional bridging composition. B—Z² is selected from any one of formulae (BZ1)-(BZ8):

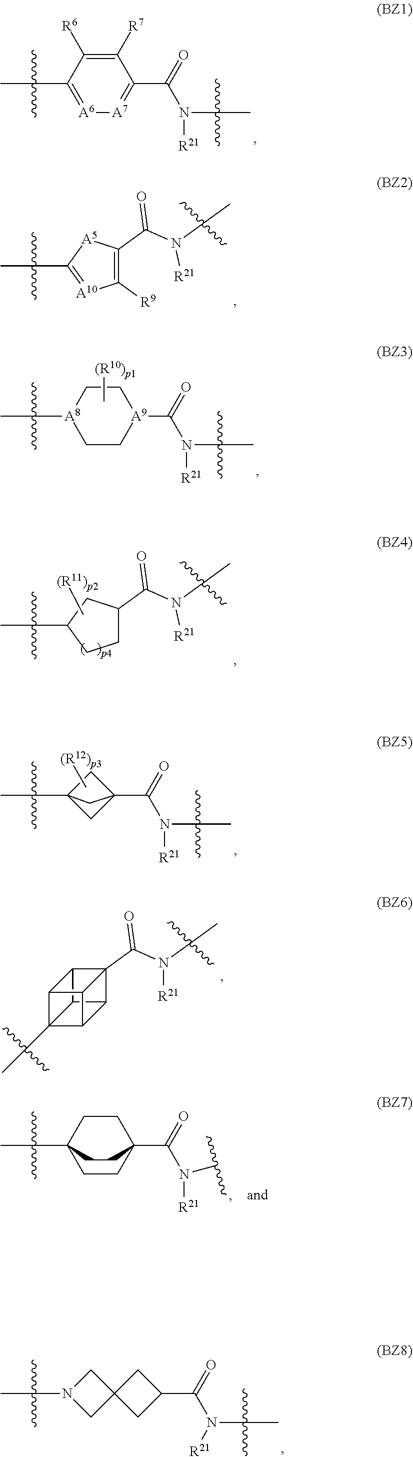

wherein:
A⁵ is selected from NR²¹, S, O, C(R⁵)₂;
A⁶-A⁹ are independently selected from N, and CR⁵; A¹⁰ is selected from N, and CR⁸;
R²¹ is selected from H, and optionally substituted (C₁-C₆)alkyl;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

In certain embodiments of the subject bifunctional bridging composition, B—Z² is of formula (BZ1). In certain embodiments of formula (BZ1), each A⁶ and A⁷ is CR⁵. In certain cases, at least one of A⁶ and A⁷ is N. In certain cases, A⁶ is CR⁵ and A⁷ is N. In certain other cases, A⁶ is N and A⁷ is CR⁵. In certain cases. R⁵ is H. In certain cases, R⁵ is halogen. In certain cases, the halogen is F or Cl. In certain cases, R⁵ is (C₁-C₃)alkyl. In certain cases, R⁵ is methyl. In certain cases, each of R⁶ and R⁷ is H. In certain other cases, at least one of R⁶ and R⁷ is a substituent other than H. In certain cases, at least one of R⁶ and R⁷ is halogen. In certain cases, the halogen is F or Cl. In certain cases, at least one of R⁶ and R⁷ is (C₁-C₃)alkyl. In certain cases, at least one of R⁶ and R⁷ is methyl. In certain embodiments of formula (BZ1), R²¹ is H. In certain other embodiments, R²¹ is (C₁-C₃)alkyl. In certain cases, R²¹ is methyl.

In certain embodiments of the subject bifunctional bridging composition, B—Z² is of formula (BZ2). In certain embodiments of formula (BZ2). A⁵ is NR²¹, where R²¹ is selected from H or (C₁-C₃)alkyl, e.g., methyl. In certain cases, A⁵ is S. In certain cases, A⁵ is O. In certain other cases, A⁵ is C(R⁵)₂. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, A¹⁰ is CR⁸ and each of R⁸ and R⁹ is H. In certain other cases, A¹⁰ is CR⁸ and at least one of R⁸ and R⁹ is a substituent other than H. In certain cases, A¹⁰ is CR⁸ and at least one of R⁸ and R⁹ is halogen. In certain cases, the halogen is F or Cl. In certain cases, at least one of R⁸ and R⁹ is (C₁-C₃)alkyl. In certain cases, A¹⁰ is CR⁸ and at least one of R⁸ and R⁹ is methyl. In certain embodiments of formula (BZ2), R²¹ is H. In certain other embodiments, R²¹ is (C₁-C₃)alkyl. In certain cases, R²¹ is methyl. In certain embodiments of formula (BZ2), A¹⁰ is CR⁸, where R⁸ is selected from H or (C₁-C₃)alkyl, e.g., methyl. In certain embodiments of formula (BZ2), A¹⁰ is CH. In cases of formula (BZ2), A¹⁰ is N. In certain embodiments of formula (BZ2). A⁵ is NR²¹ and A¹⁰ is CR⁸, where R²¹ and R⁸ are independently selected from H or (C₁-C₃)alkyl, e.g., methyl. In certain embodiments of formula (BZ2), A⁵ is NR²¹ and A¹⁰ is N. In certain embodiments of formula (BZ2), A⁵ is S and A¹⁰ is N.

In certain embodiments of the subject bifunctional bridging composition, B—Z² is of formula (BZ3). In certain embodiments of formula (BZ3), each A⁸ and A⁹ is CR⁵. In certain cases, at least one of A⁸ and A⁹ is N. In certain cases, A⁸ is CR⁵ and A⁹ is N. In certain other cases, A⁸ is N and A⁹ is CR⁵. In certain cases, both of A⁸ and A⁹ are N. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, each R¹⁰ is H (or p1 is 0). In certain other cases, p1 is 1 to 10 and at least one R¹⁰ group is a substituent other than H. In certain cases, at least one R¹⁰ group is halogen. In certain cases, the halogen is F or Cl. In certain cases, at least one R¹⁰ group is (C₁-C₃)alkyl. In certain cases, at least one of R¹⁰ group is methyl. In certain embodiments of formula (BZ3), R²¹ is H. In certain other embodiments, R²¹ is (C₁-C₃)alkyl. In certain cases, R²¹ is methyl.

In certain embodiments of the subject bifunctional bridging composition, B—Z² is of formula (BZ4). In certain embodiments of formula (BZ4), p4 is 0, such that the B ring system is cyclobutyl. In certain cases, p4 is 1, such that the B ring system is a cyclopentyl. In certain cases, p4 is 2, such that the B ring system is cyclohexyl. In certain cases, p4 is 3, such that the B ring system is cycloheptyl. In certain other cases, p4 is 4, such that the B ring system is cyclooctyl. In certain cases, each R¹¹ is H (or p2 is 0). In certain other cases, p2 is 1 to 14 and at least one R¹¹ group is a substituent other than H. In certain cases, at least one R¹¹ group is halogen. In certain cases, the halogen is F or Cl. In certain cases, at least one R¹¹ group is (C₁-C₃)alkyl. In certain cases, at least one of R¹¹ group is methyl. In certain embodiments of formula (BZ4), R²¹ is H. In certain other embodiments, R²¹ is (C₁-C₃)alkyl. In certain cases, R²¹ is methyl.

In certain embodiments of the subject bifunctional bridging composition, B—Z² comprises a bicycloalkyl group and is of any of formulae (BZ5)-(BZ8). In certain embodiments of formula (BZ5), each R¹² is H (or p3 is 0). In certain other cases, p3 is 1 to 4 and at least one R¹² group is a substituent other than H. In certain cases, at least one R¹² group is halogen. In certain cases, the halogen is F or Cl. In certain cases, at least one R¹² group is (C₁-C₃)alkyl. In certain cases, at least one of R¹² group is methyl. In certain embodiments of formula (BZ5). R²¹ is H. In certain other embodiments, R²¹ is (C₁-C₃)alkyl. In certain cases, R²¹ is methyl. In certain embodiments of any of formulae (BZ6)-(BZ8), R²¹ is H. In certain other embodiments, R²¹ is (C₁-C₃)alkyl. In certain cases, R²¹ is methyl.

In certain embodiments of the subject bifunctional bridging composition, B—Z² is:

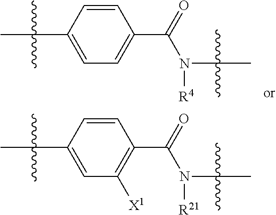

wherein X¹ is halogen. In certain cases, the halogen is F. In certain cases, the halogen is Cl. In certain cases, the halogen is bromide.

In certain embodiments of the subject bifunctional bridging composition, T¹-Z¹—B is selected from:

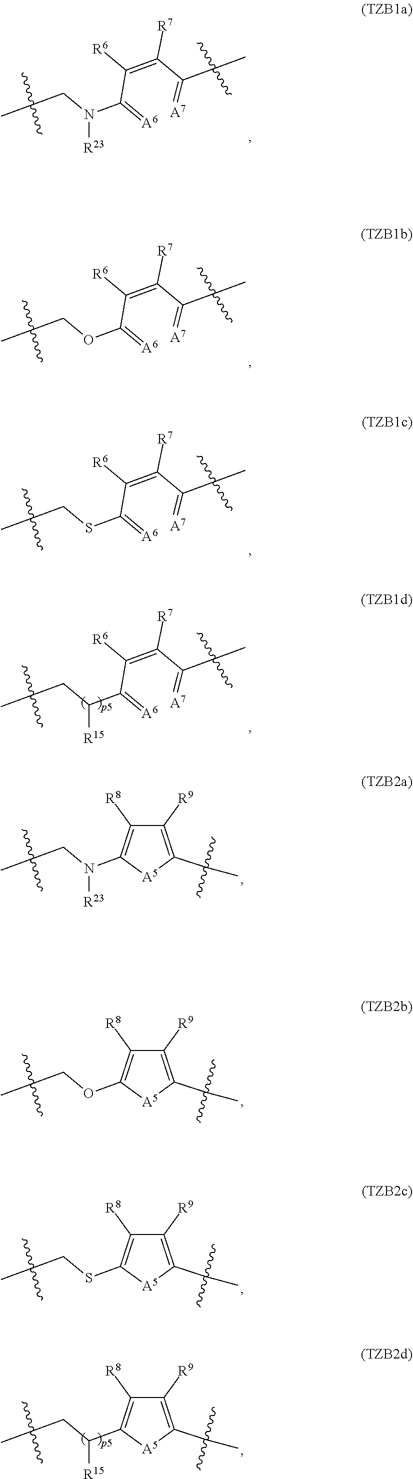

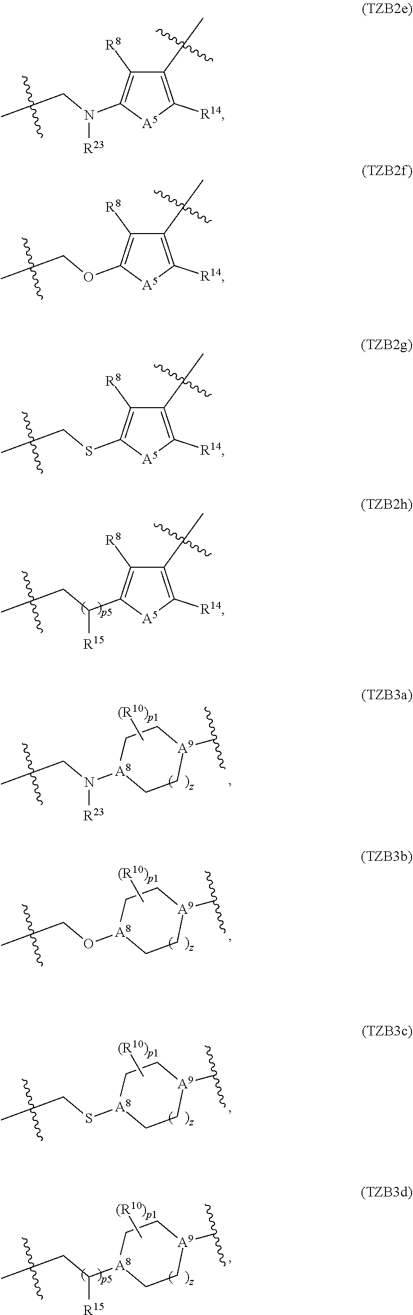

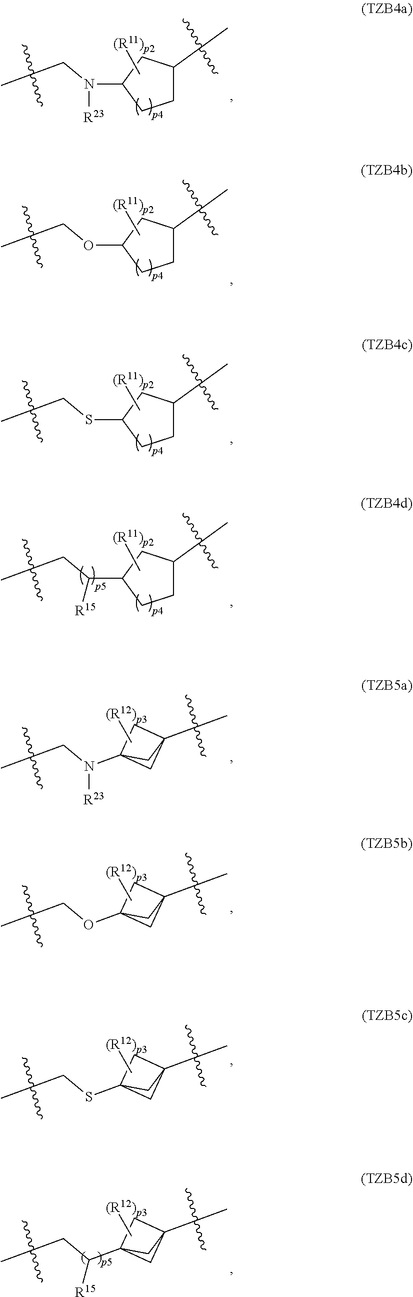

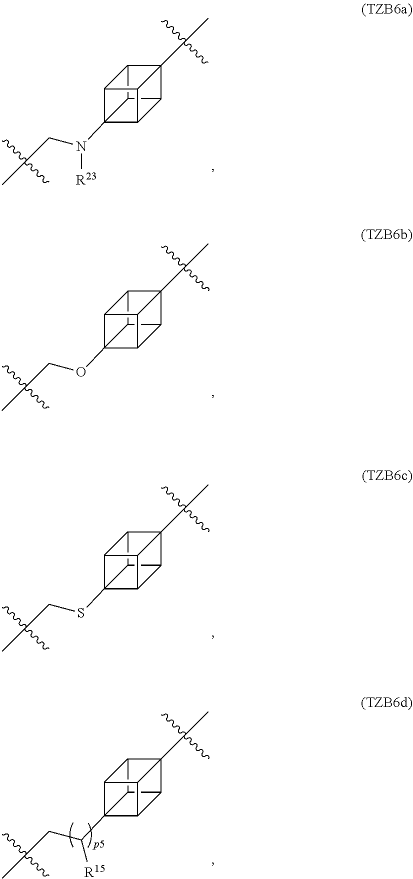

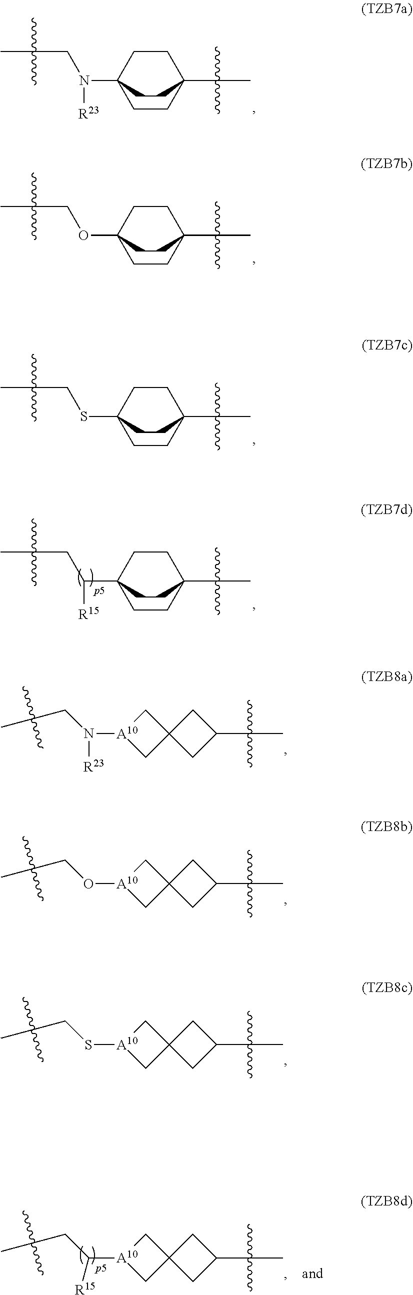

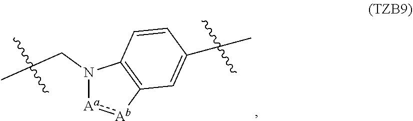

wherein:

A⁵ is selected from NR²¹, S, O, C(R⁵)₂;

A⁶-A¹⁰ are independently selected from N, and CR⁵;

R²³ is H, optionally substituted (C₁-C₆)alkyl, or R²³ forms a 5 or 6 membered cycle together with an atom of the adjacent cycle;
each R⁵ to R¹² and R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

R¹⁵ is H, optionally substituted (C₁-C₆)alkyl, or R¹⁵ forms a 5 or 6 membered cycle together with an atom of the adjacent cycle;

[symbol] is a single bond or a double bond;

wherein when [symbol] is a single bond A^{a} is selected from C(R⁵)₂, and C═O, and A^{b} is selected from C(R⁵)₂, and NR²¹; and

when [symbol] is a double bond A^{a} is CR⁵, and A^{b} is selected from CR⁵ and N

p1 is 0 to 10;

p2 is 0 to 14;

p3 is 0 to 4;

p4 0 to 4; and

p5 is 1 to 3.

In certain embodiments of the subject bifunctional bridging composition, T¹-Z¹—B is of any one of formulae (TZB1a)-(TZB1d), and each of A⁶-A⁷, and R⁶-R⁷ are as defined for formula (BZ1). In certain embodiments of formula (TZB1a) or (TZB1d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent aryl or heteroaryl ring. In certain embodiments R²³ or R¹⁵ forms a fused 6-membered cycle with an atom of the adjacent aryl or heteroaryl ring. In certain embodiments of formula (TZB1d), p5 is 1. In certain embodiments, p5 is 2. In certain other embodiments, p5 is 3.

In certain embodiments of the subject bifunctional bridging composition, T¹-Z¹—B is of any one of formulae (TZB2a)-(TZB2h), and each of A⁵, and R⁸-R⁹ are as defined for formula (BZ2). In certain embodiments, R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent 5-membered ring. In certain embodiments R²³ or R¹⁵ forms a fused 6-membered cycle with an atom of the adjacent 5-membered ring. In certain embodiments of formula (TZB2d) or (TZB2h), p5 is 1. In certain embodiments, p5 is 2. In certain other embodiments, p5 is 3.

In certain embodiments of the subject bifunctional bridging composition, T¹-Z¹—B is of any one of formulae (TZB3a)-(TZB3d), and each of A⁸-A⁹, R¹⁰, z and p1 are as defined for formula (BZ3). In certain embodiments of formula (TZB3a) or (TZB3d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent 6-membered ring. In certain embodiments R²³ or R¹⁵ forms a fused 6-membered cycle with an atom of the adjacent 6-membered ring. In certain embodiments of formula (TZB3d), p5 is 1. In certain embodiments, p5 is 2. In certain other embodiments, p5 is 3.

In certain embodiments of the subject bifunctional bridging composition. T¹-Z¹—B is of any one of formulae (TZB4a)-(TZB4d), and each of R¹¹, p2 and p4 are as defined for formula (BZ4). In certain embodiments of formula (TZB4a) or (TZB4d), R²¹ or R¹⁵ is H. In certain other embodiments, R²¹ or R¹⁵ is optionally substituted (C₁-C₃)alkyl. In certain cases, R²² or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent ring. In certain embodiments R²¹ or R¹⁵ forms a fused 6-membered cycle with an atom of the adjacent ring. In certain embodiments of formula (TZB4d), p5 is 1. In certain embodiments, p5 is 2. In certain other embodiments, p5 is 3.

In certain embodiments of the subject bifunctional bridging composition, T¹-Z¹—B is selected from any one of formulae (TZB5a)-(TZB5d), (TZB6a)-(TZB6d), (TZB7a)-(TZB7d), and (TZB8a)-(TZB8d), and each of R², and p3 are as defined for formula (BZ5). In certain embodiments R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments of formula (TZB4d), (TZB6d), (TZB7d), or (TZB8d), p5 is 1. In certain embodiments, p5 is 2. In certain other embodiments, p5 is 3.

In certain embodiments of the subject bifunctional bridging composition. T¹-Z¹—B is of formula (TZB9). In certain cases, the compound of formula (TZB9) is of any one of the following structures:

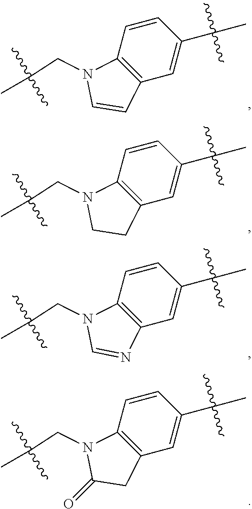

In certain embodiments of the subject bifunctional bridging composition. T¹-Z¹ is optionally substituted (C₁-C₆)alkylene, and A-T¹-Z¹—B— is selected from one of formulae (AB1)-(AB6):

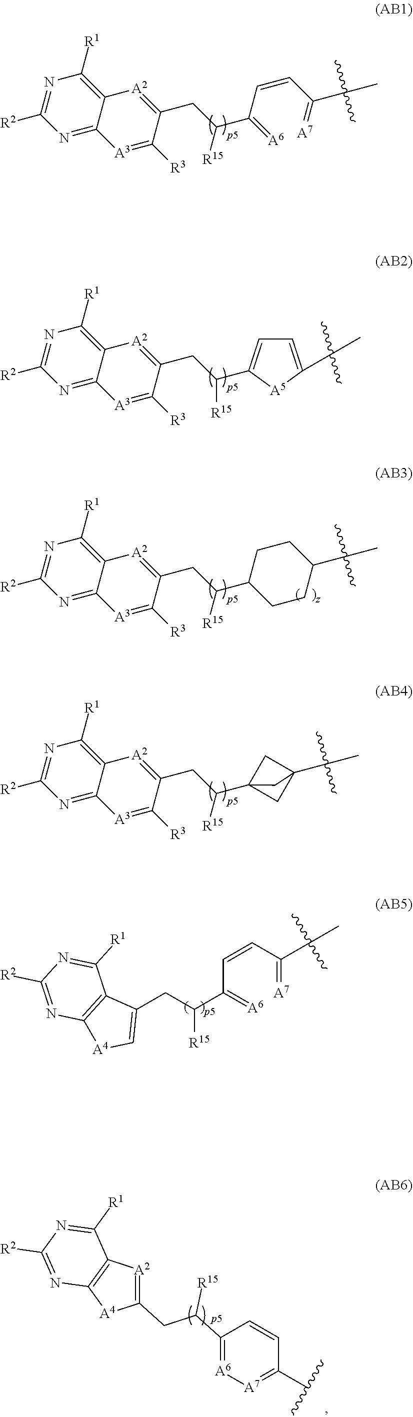

or a tautomer thereof, wherein:

A²-A⁷, R¹-R³ and z are as described herein above;

each R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each p5 is independently 1 to 3.

In certain embodiments of the subject bifunctional bridging composition. A-T¹-Z¹—B— is of formula (AB1), and each of A²-A³, A⁶-A⁷, and R¹-R³ are as described herein. In certain instances, R¹ is OH or NH₂. In certain instances, R² is NH₂, CH, or CH₂OH. In certain instances. R³ is H. In certain instances, both A² and A³ are N. In certain other instances, both A² and A³ are CH. In certain instances, both A⁶ and A⁷ are CH. In certain embodiments of formula (AB1), R¹⁵ is H. In certain other embodiments. R¹⁵ is optionally substituted (C₁-C₃)alkyl. In certain cases, R¹⁵ is methyl. In certain embodiments, R¹⁵ is an alkyne moiety of formula —(CH₂)nCCH, where n is 1 or 2. In certain embodiments R¹⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB1), p5 is 1. In certain embodiments, p5 is 2. In certain other embodiments, p5 is 3.

In certain embodiments of formula (AB1), the bifunctional bridging composition is selected from one of the following:

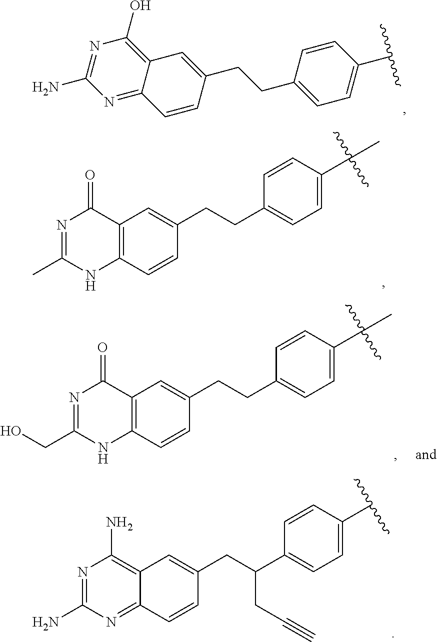 [SMILES: *c1ccc(CCc2ccc3nc(N)nc(O)c3c2)cc1 ; *c1ccc(CCc2ccc3[nH]c(C)nc(=O)c3c2)cc1 ; *c1ccc(CCc2ccc3[nH]c(CO)nc(=O)c3c2)cc1 ; *c1ccc(C(CC#C)Cc2ccc3nc(N)nc(N)c3c2)cc1]

In certain embodiments of the subject bifunctional bridging composition, A-T¹-Z¹—B— is of formula (AB2), and each of A²-A³, A⁵, and R¹-R³ are as described herein. In certain instances, R¹ is OH or NH₂. In certain instances. R² is NH₂, CH₃, or CH₂OH. In certain instances, R³ is H. In certain instances, both A² and A³ are N. In certain other instances, both A² and A³ are CH. In certain instances, A⁵ is S or O. In certain embodiments of formula (AB2), R¹⁵ is H. In certain other embodiments, R¹⁵ is optionally substituted (C₁-C₃)alkyl. In certain cases, R¹⁵ is methyl. In certain embodiments, R¹⁵ is an alkyne moiety of formula —(CH₂)nCCH, where n is 1 or 2. In certain embodiments R¹⁵ forms a fused 5-membered cycle with an atom of the adjacent 5-membered ring. In certain embodiments R¹⁵ forms a fused 6-membered cycle with an atom of the adjacent 5-membered ring. In certain embodiments of formula (AB2), p5 is 1. In certain embodiments, p5 is 2. In certain other embodiments, p5 is 3.

In certain embodiments of the subject bifunctional bridging composition, A-T¹-Z¹—B— is of formula (AB3), and each of A²-A³, R¹-R³ and z are as described herein. In certain instances, R¹ is OH or NH₂. In certain instances, R² is NH₂. CH₃, or CH₂OH. In certain instances, R³ is H. In certain instances, both A² and A³ are N. In certain other instances, both A² and A³ are CH. In certain instances, z is 1. In certain embodiments of formula (AB3), R¹⁵ is H. In certain other embodiments, R¹⁵ is optionally substituted (C₁-C₃)alkyl. In certain cases, R¹⁵ is methyl. In certain embodiments, R¹⁵ is an alkyne moiety of formula —(CH₂)nCCH, where n is 1 or 2. In certain embodiments R¹⁵ forms a fused 5-membered cycle with an atom of the adjacent cycloalkyl ring. In certain embodiments R¹⁵ forms a fused 6-membered cycle with an atom of the adjacent cycloalkyl ring. In certain embodiments of formula (AB1), p5 is 1. In certain embodiments, p5 is 2. In certain other embodiments, p5 is 3.

In certain embodiments of formula (AB3), the bifunctional bridging composition includes the following structure:

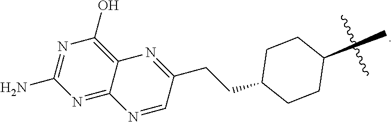 [SMILES: C[C@H]1CC[C@H](CCc2cnc3nc(N)nc(O)c3n2)CC1]

In certain embodiments of the subject bifunctional bridging composition. A-T¹-Z¹—B— is of formula (AB4), and each of A²-A³, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain embodiments of formula (AB4), R¹⁵ is H. In certain other embodiments. R¹⁵ is optionally substituted (C₁-C₃)alkyl. In certain cases, R¹⁵ is methyl. In certain embodiments, R¹⁵ is an alkyne moiety of formula —(CH₂)nCCH, where n is 1 or 2. In certain embodiments of formula (AB4), p5 is 1. In certain embodiments, p5 is 2. In certain other embodiments, p5 is 3.

In certain embodiments of the subject bifunctional bridging composition, A-T¹-Z¹—B— is of formula (AB5) or (AB6), and each of A², A⁴, A⁶-A⁷, and R¹-R² are as described herein. In certain instances, R¹ is OH or NH₂. In certain instances, R² is NH₂, CH₃, or CH₂OH. In certain instances of formula (AB6), A² is CH. In certain other instances of formula (AB5) and (AB6), A⁴ is NH. In certain instances, both A⁶ and A⁷ are CH. In certain instances, A⁶ is CH and A⁷ are N. In certain embodiments of formula (AB5) or (AB6), R¹⁵ is H. In certain other embodiments, R¹⁵ is optionally substituted (C₁-C₃)alkyl. In certain cases, R¹⁵ is methyl. In certain embodiments, R¹⁵ is an alkyne moiety of formula —(CH₂)nCCH, where n is 1 or 2. In certain embodiments R¹⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB5) or (AB6), p5 is 1. In certain embodiments, p5 is 2. In certain other embodiments, p5 is 3.

In certain embodiments, formula (AB5) or (AB6) is selected from the following structures:

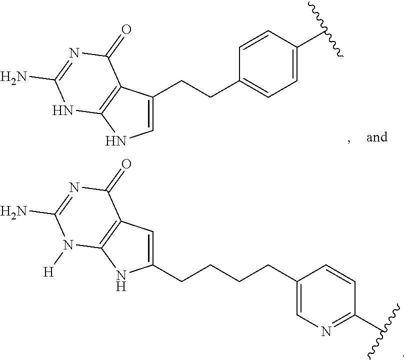

In certain embodiments of the subject bifunctional bridging composition, A-T¹-Z¹—B— is selected from one of formulae (AB7)-(AB12):

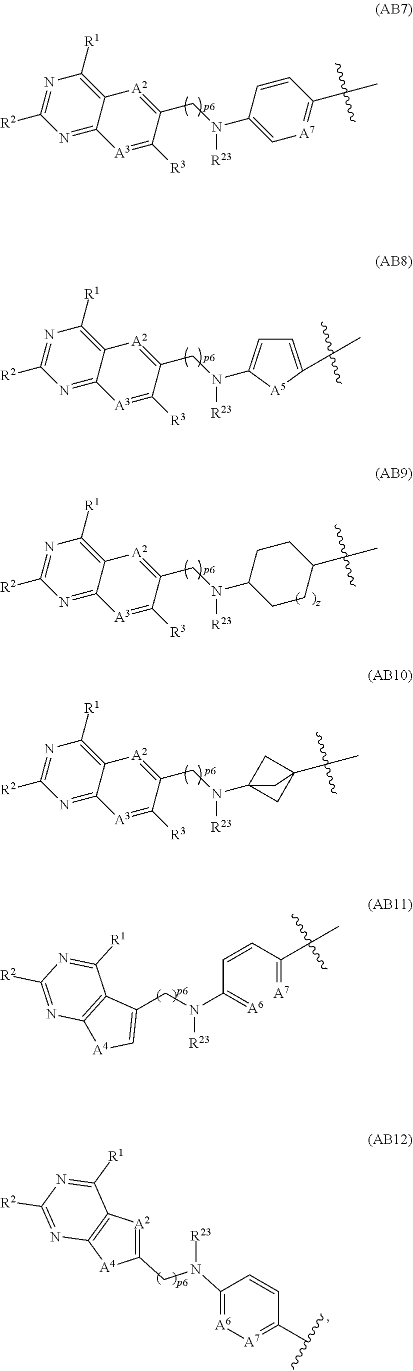

or a tautomer thereof, wherein:

A²-A⁷, R¹-R³ and z are as described herein above;

R²³ is H, optionally substituted (C₁-C₆)alkyl, or R²³ forms a 5 or 6 membered cycle together with an atom of the adjacent cycle;

each p6 is independently 1 to 3.

In certain embodiments of formula (AB7) to (AB12), R²³ is H. In certain other embodiments, R²³ is optionally substituted (C₁-C₃)alkyl. In certain cases, R²³ is methyl. In certain embodiments, R²³ is an alkyne moiety of formula —(CH₂)ₙCCH, where n is 1 or 2. In certain embodiments R²³ forms a fused 5-membered cycle with an atom of the adjacent aryl or heteroaryl ring. In certain embodiments R²³ forms a fused 6-membered cycle with an atom of the adjacent aryl or heteroaryl ring. In certain embodiments of formula (AB7) to (AB12), p6 is 1. In certain embodiments, p6 is 2. In certain other embodiments, p6 is 3.

In certain embodiments of the subject bifunctional bridging composition, A-T¹-Z¹—B— is selected from one of formulae (AB13)-(AB18):

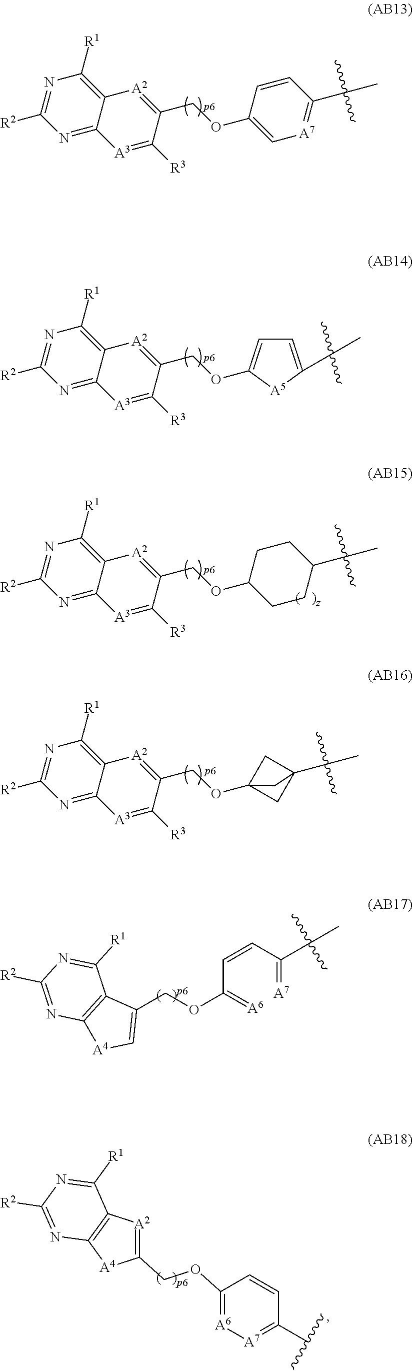

or a tautomer thereof, wherein:

A²-A⁷, R¹-R³ and z are as described herein above; and

each p6 is independently 1 to 3.

In certain embodiments of formula (AB13) to (AB18), p6 is 1. In certain embodiments, p6 is 2. In certain other embodiments, p6 is 3.

In certain embodiments of the subject bifunctional bridging composition, A-T¹-Z¹—B— is selected from one of formulae (AB19)-(AB24):

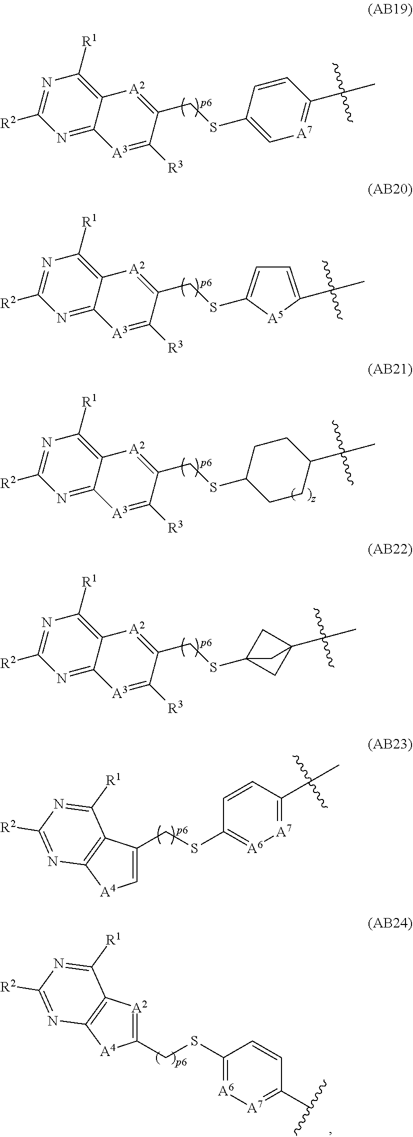

or a tautomer thereof, wherein:

A²-A⁷, R¹-R³ and z are as described herein above; and each p6 is independently 1 to 3.

In certain embodiments of formula (AB19) to (AB24), p6 is 1. In certain embodiments, p6 is 2. In certain other embodiments, p6 is 3.

In some embodiments, the subject bifunctional bridging composition comprises a cell surface folate receptor ligand selected from one of the following structures:

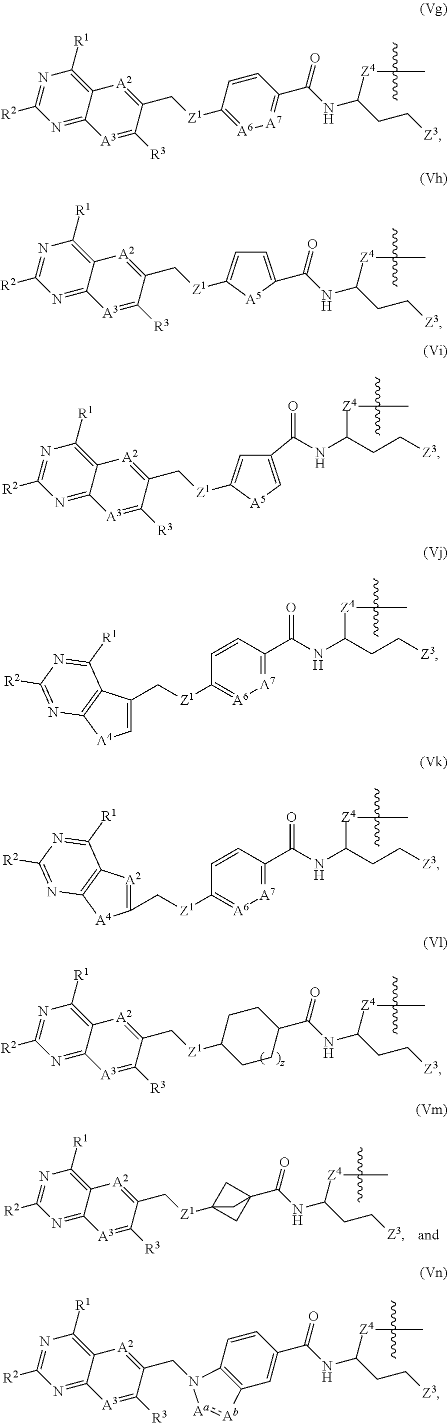

wherein:

A⁵ is selected from NR²¹, S, O, C(R⁵)₂;

A⁶ and A⁷ are independently selected from N, and, CR⁵;

z is 0 to 3;

[symbol] is a single bond or a double bond;
wherein [symbol] is a single bond A^{a} is selected from C(R⁵)₂, and C═O. and A^{b} is selected from C(R⁵)₂, and NR²¹; and

when [symbol] is a double bond A^{a} is CR⁵, and A^{b} is selected from CR⁵ and N; and

wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

In certain embodiments, the subject bifunctional bridging composition comprises a cell surface folate receptor ligand selected from one of the following structures:

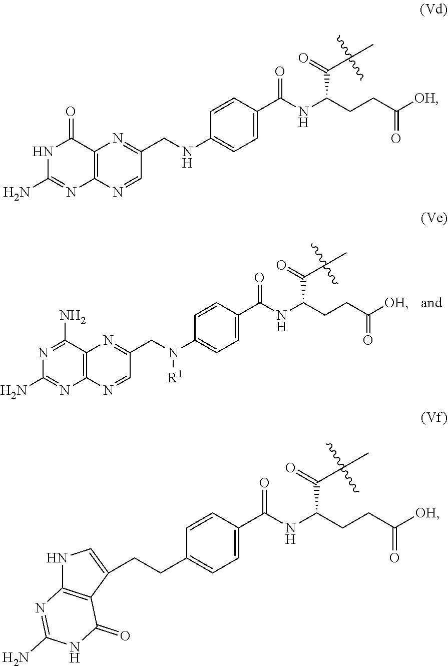

wherein R¹ is —H or —CH₃.

In certain embodiments, the cell surface folate receptor ligand is of formula (Vg) and each of R¹-R³, A²-A³, A⁶-A⁷, Z¹ and Z³-Z⁴ are as described herein above.

In certain embodiments, the cell surface folate receptor ligand is of formula (Vh) or (Vi) and each of R¹-R³, A²-A³, A⁵, Z¹ and Z³-Z⁴ are as described herein above.

In certain embodiments, the cell surface folate receptor ligand is of formula (Vj) or (Vk) and each of R¹-R², A², A⁴, A⁶-A⁷, Z¹ and Z³-Z⁴ are as described herein above.

In certain embodiments, the cell surface folate receptor ligand is of formula (Vl) and each of R¹-R³, A²-A³, z, Z¹ and Z³-Z⁴ are as described herein above.

In certain embodiments, the cell surface folate receptor ligand is of formula (Vm) and each of R¹-R³, A²-A³, Z¹ and Z³-Z⁴ are as described herein above.

In certain embodiments, the cell surface folate receptor ligand is of formula (Vn) and each of R¹-R³, A²-A³, A^{a}-A^{b}, and Z³-Z⁴ are as described herein above.

In some embodiments, the cell surface folate receptor ligand selected from one of the following structures:

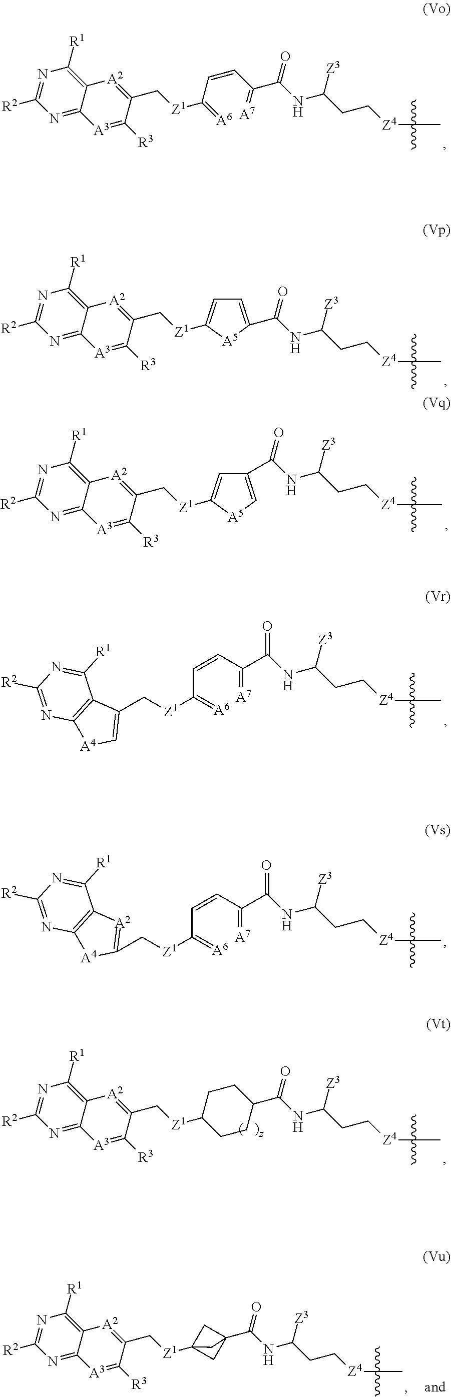

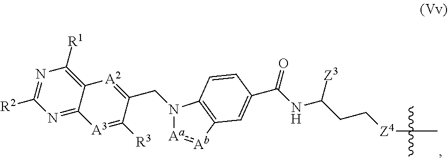

wherein:

A⁵ is selected from NR²¹, S, O, C(R²¹)₂;

A⁶ and A⁷ are each independently selected from N, and, CR²¹;

z is 0 to 3;

[symbol] is a single bond or a double bond;

wherein when [symbol] is a single bond A^{a} is selected from C(R²¹)₂, and C═O, and A^{b} is selected from C(R²¹)₂, and NR²¹; and

when [symbol] is a double bond A^{a} is CR²¹; and A^{b} is selected from CR²¹ and N.

In certain embodiments, the cell surface folate receptor ligand selected from one of the following structures:

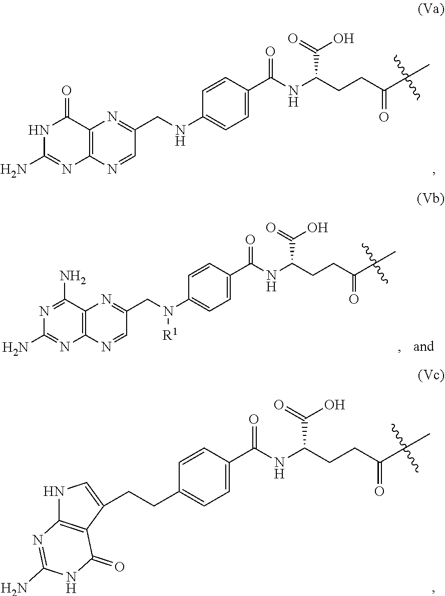

wherein R¹ is —H or —CH₃.

In certain embodiments, the cell surface folate receptor ligand is of formula (Vo) and each of R¹-R³, A²-A³, A⁶-A⁷, Z¹ and Z³-Z⁴ are as described herein above.

In certain embodiments, the cell surface folate receptor ligand is of formula (Vp) or (Vq) and each of R¹-R³, A²-A³, A⁵, Z¹ and Z³-Z⁴ are as described herein above.

In certain embodiments, the cell surface folate receptor ligand is of formula (Vr) or (Vs) and each of R¹-R², A², A⁴, A⁶-A⁷, Z¹ and Z³-Z⁴ are as described herein above.

In certain embodiments, the cell surface folate receptor ligand is of formula (Vt) and each of R¹-R³, A²-A³, z, Z¹ and Z³-Z⁴ are as described herein above.

In certain embodiments, the cell surface folate receptor ligand is of formula (Vu) and each of R¹-R³, A²-A³, Z¹ and Z³-Z⁴ are as described herein above.

In certain embodiments, the cell surface folate receptor ligand is of formula (Vv) and each of R¹-R³, A²-A³, A^{a}-A^{b}, and Z³-Z⁴ are as described herein above.

In certain embodiments, the cell surface folate receptor ligand which can be utilized in the preparation of compounds of this disclosure are shown in tables 3A-3B.

TABLE 3A
Exemplary cell surface folate receptor binding moieties

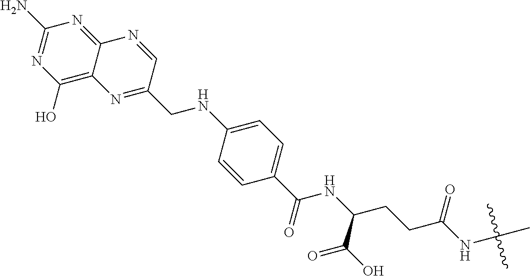 [SMILES: *NC(=O)CC[C@H](NC(=O)c1ccc(NCc2cnc3nc(N)nc(O)c3n2)cc1)C(=O)O]

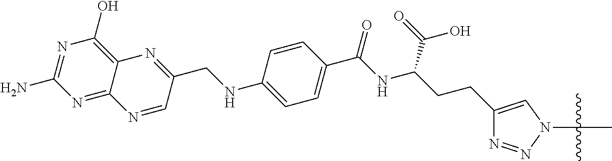 [SMILES: *n1cc(CC[C@H](NC(=O)c2ccc(NCc3cnc4nc(N)nc(O)c4n3)cc2)C(=O)O)nn1]

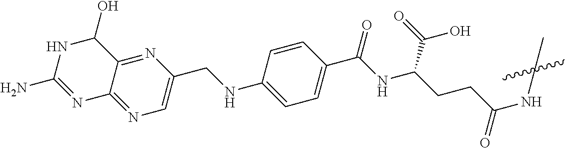 [SMILES: *NC(=O)CC[C@H](NC(=O)c1ccc(NCc2cnc3c(n2)C(O)NC(N)=N3)cc1)C(=O)O]

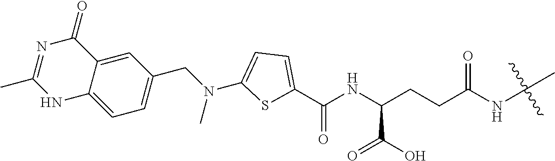 [SMILES: *NC(=O)CC[C@H](NC(=O)c1ccc(N(C)Cc2ccc3[nH]c(C)nc(=O)c3c2)s1)C(=O)O]

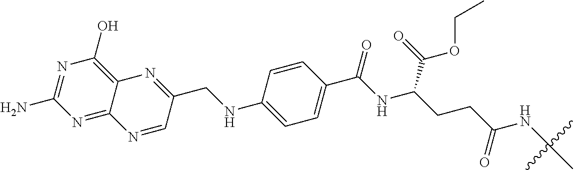 [SMILES: *NC(=O)CC[C@H](NC(=O)c1ccc(NCc2cnc3nc(N)nc(O)c3n2)cc1)C(=O)OCC]

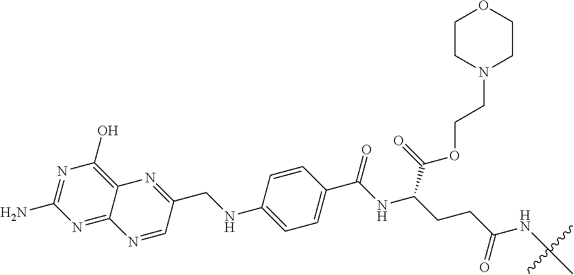 [SMILES: *NC(=O)CC[C@H](NC(=O)c1ccc(NCc2cnc3nc(N)nc(O)c3n2)cc1)C(=O)OCCN1CCOCC1]

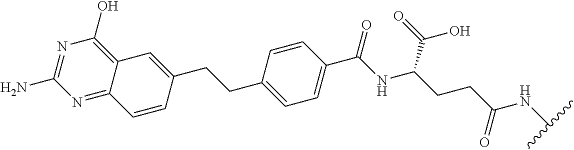 [SMILES: *NC(=O)CC[C@H](NC(=O)c1ccc(CCc2ccc3nc(N)nc(O)c3c2)cc1)C(=O)O]

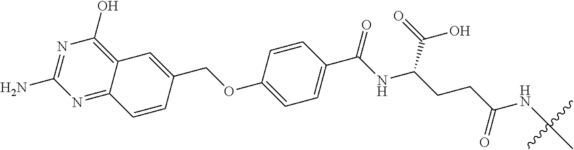 [SMILES: *NC(=O)CC[C@H](NC(=O)c1ccc(OCc2ccc3nc(N)nc(O)c3c2)cc1)C(=O)O]

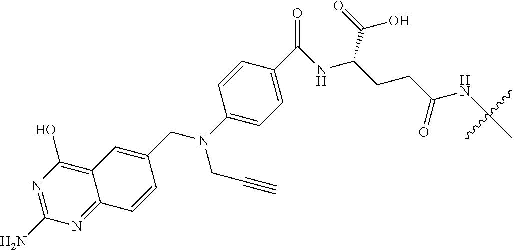 [SMILES: *NC(=O)CC[C@H](NC(=O)c1ccc(N(CC#C)Cc2ccc3nc(N)nc(O)c3c2)cc1)C(=O)O]

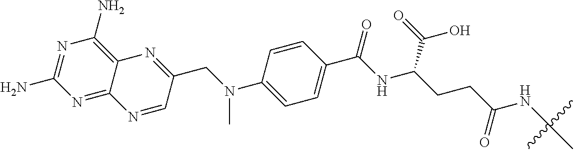 [SMILES: *NC(=O)CC[C@H](NC(=O)c1ccc(N(C)Cc2cnc3nc(N)nc(N)c3n2)cc1)C(=O)O]

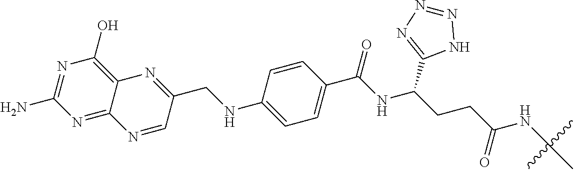 [SMILES: *NC(=O)CC[C@H](NC(=O)c1ccc(NCc2cnc3nc(N)nc(O)c3n2)cc1)c1nnn[nH]1]

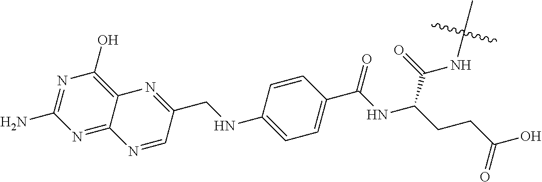 [SMILES: *NC(=O)[C@H](CCC(=O)O)NC(=O)c1ccc(NCc2cnc3nc(N)nc(O)c3n2)cc1]

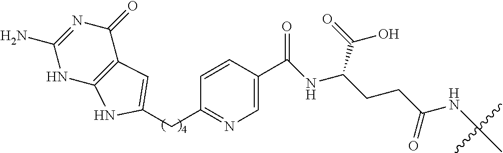

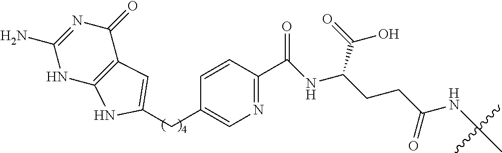

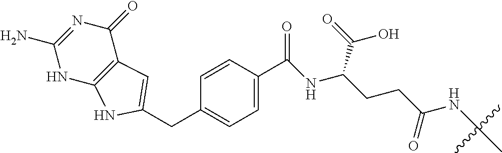 [SMILES: *NC(=O)CC[C@H](NC(=O)c1ccc(Cc2cc3c(=O)nc(N)[nH]c3[nH]2)cc1)C(=O)O]

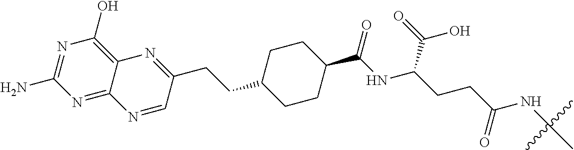

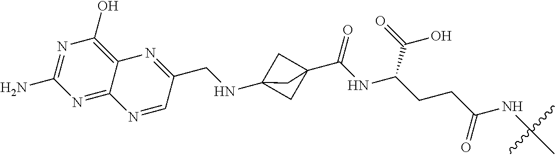 [SMILES: *NC(=O)CC[C@H](NC(=O)C12CC(NCc3cnc4nc(N)nc(O)c4n3)(C1)C2)C(=O)O]

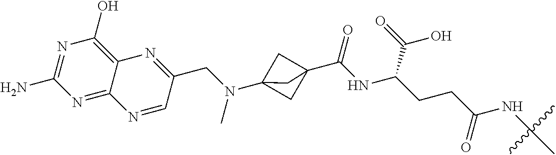 [SMILES: *NC(=O)CC[C@H](NC(=O)C12CC(N(C)Cc3cnc4nc(N)nc(O)c4n3)(C1)C2)C(=O)O]

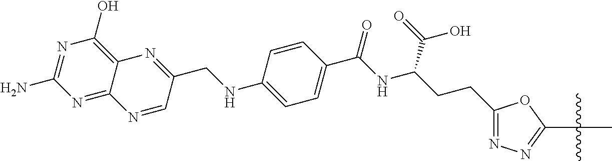 [SMILES: *c1nnc(CC[C@H](NC(=O)c2ccc(NCc3cnc4nc(N)nc(O)c4n3)cc2)C(=O)O)o1]

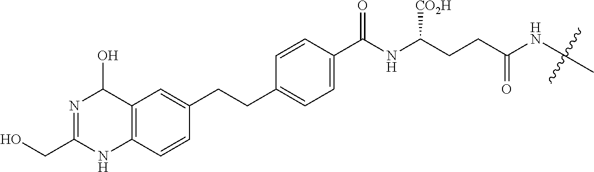 [SMILES: *NC(=O)CC[C@H](NC(=O)c1ccc(CCc2ccc3c(c2)C(O)N=C(CO)N3)cc1)C(=O)O]

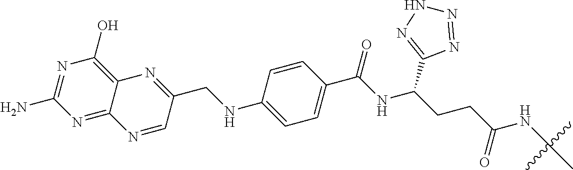 [SMILES: *NC(=O)CC[C@H](NC(=O)c1ccc(NCc2cnc3nc(N)nc(O)c3n2)cc1)c1nn[nH]n1]

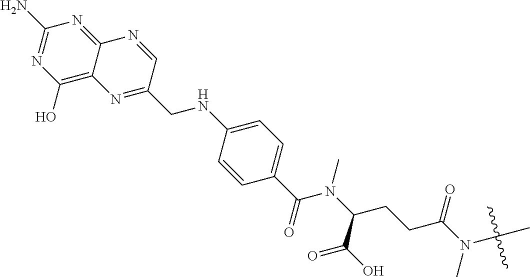 [SMILES: *N(C)C(=O)CC[C@@H](C(=O)O)N(C)C(=O)c1ccc(NCc2cnc3nc(N)nc(O)c3n2)cc1]

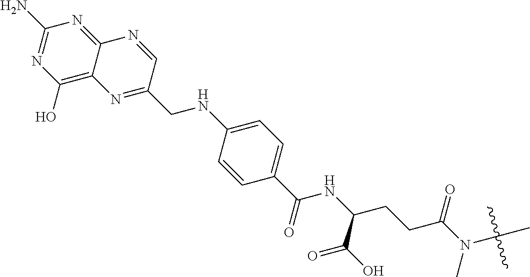 [SMILES: *N(C)C(=O)CC[C@H](NC(=O)c1ccc(NCc2cnc3nc(N)nc(O)c3n2)cc1)C(=O)O]

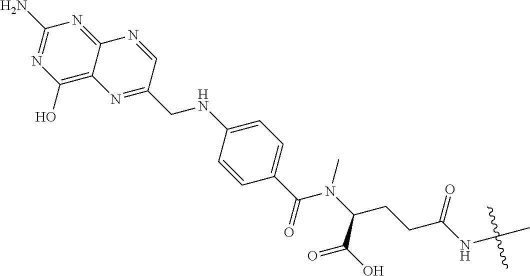 [SMILES: *NC(=O)CC[C@@H](C(=O)O)N(C)C(=O)c1ccc(NCc2cnc3nc(N)nc(O)c3n2)cc1]

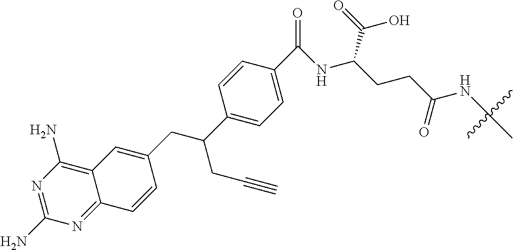 [SMILES: *NC(=O)CC[C@H](NC(=O)c1ccc(C(CC#C)Cc2ccc3nc(N)nc(N)c3c2)cc1)C(=O)O]

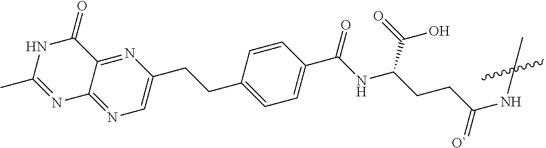

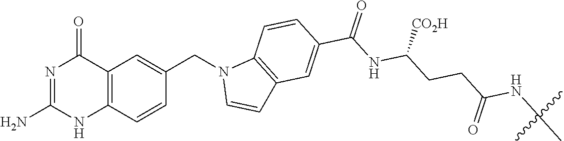 [SMILES: *NC(=O)CC[C@H](NC(=O)c1ccc2c(ccn2Cc2ccc3[nH]c(N)nc(=O)c3c2)c1)C(=O)O]

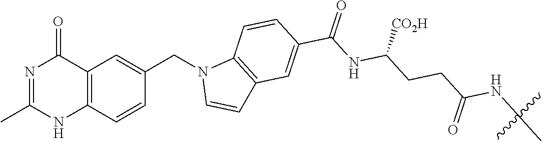 [SMILES: *NC(=O)CC[C@H](NC(=O)c1ccc2c(ccn2Cc2ccc3[nH]c(C)nc(=O)c3c2)c1)C(=O)O]

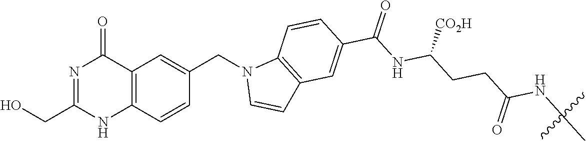 [SMILES: *NC(=O)CC[C@H](NC(=O)c1ccc2c(ccn2Cc2ccc3[nH]c(CO)nc(=O)c3c2)c1)C(=O)O]

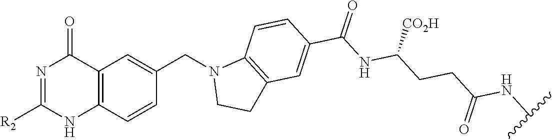

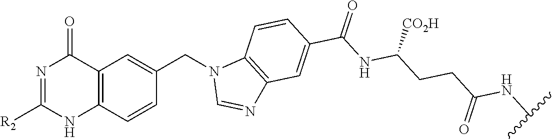

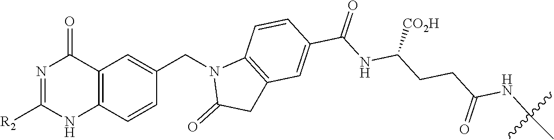

TABLE 3B
cell surface folate receptor binding moieties

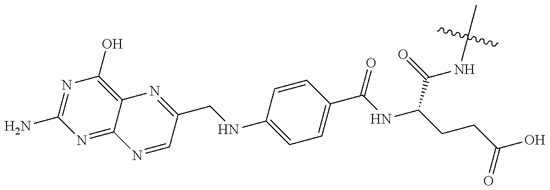 [SMILES: *NC(=O)[C@H](CCC(=O)O)NC(=O)c1ccc(NCc2cnc3nc(N)nc(O)c3n2)cc1]

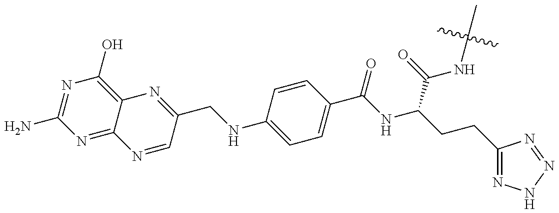 [SMILES: *NC(=O)[C@H](CCc1nn[nH]n1)NC(=O)c1ccc(NCc2cnc3nc(N)nc(O)c3n2)cc1]

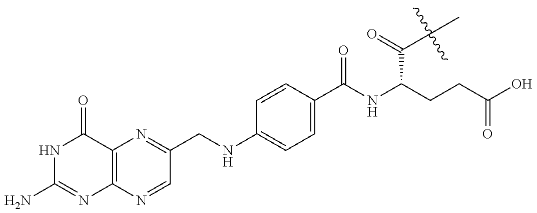 [SMILES: *C(=O)[C@H](CCC(=O)O)NC(=O)c1ccc(NCc2cnc3nc(N)[nH]c(=O)c3n2)cc1]

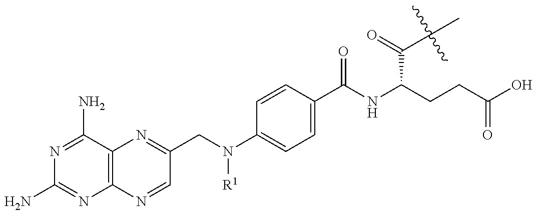

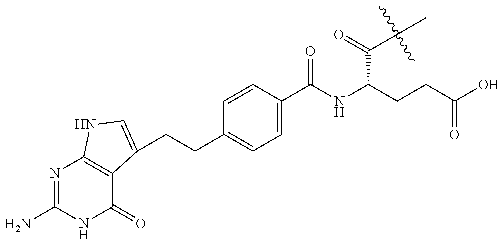 [SMILES: *C(=O)[C@H](CCC(=O)O)NC(=O)c1ccc(CCc2c[nH]c3nc(N)[nH]c(=O)c23)cc1]

In Tables 3A or 3B, the [symbol] can represent the point of attachment to -L-Y.

In certain embodiments of the compound of formula (Id), (IIIA), or (IIIB), n is 1. In certain cases, n is at least 2. In certain other cases, n is 2 to 20, such as 2 to 15, 2 to 10, 2 to 8, 2 to 6, or 2 to 4. In certain cases, n is 2 to 6. In certain other cases, n is 2 or 3.

5.2.2. Bridging Moieties that Bind Virus Composition

As used herein, a “bridging moiety” includes any moiety that specifically binds to a viral composition, for example, a viral particle, viral capsid, viral envelope or viral protein (e.g., a viral capsid protein or envelope protein), wherein the binding is not via a covalent linkage.

Any suitable moiety that binds a viral particle, viral capsid, viral envelope or viral protein (e.g., a viral capsid protein or envelope protein) can be adapted for use in the bifunctional bridging compositions of this disclosure.

In certain embodiments, a bridging moiety is a polypeptide that specifically binds a viral composition. In some embodiments, the bridging moiety is a polypeptide that binds to a viral composition. e.g., a virus particle, virus capsid, virus envelope, or a viral protein, for example, a viral capsid protein or viral envelope protein. In certain aspects, the bridging composition binds the viral capsid protein or a viral envelope protein, when the viral protein is part of a virus particle.

In certain embodiments, a bridging moiety is an antibody or antibody fragment (e.g., an antigen binding fragment of an antibody) that specifically binds a viral composition. In certain embodiments, a bridging moiety that binds a viral protein may also bind a viral particle, for example, via binding to the viral protein incorporated in a viral particle. Likewise, in certain embodiments, a bridging moiety that binds a viral particle may also bind a viral protein even if the viral protein is not incorporated in a viral particle. The viral particle can be an AAV virus particle. The viral protein can be a AAV capsid protein.

In some embodiments, for example, bridging compositions described herein comprise bridging moieties that specifically bind to an AAV composition, e.g., an AAV particle, AAV capsid, or AAV viral protein (e.g., an AAV capsid protein, for example, a VP1, VP2 or VP3 protein.

An antibody or antigen binding fragment that may be utilized in connection with the modified viral compositions provided herein, e.g., in connection with the bridging compositions and bridging moieties presented herein, includes, without limitation, monoclonal antibodies, antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies).

An antibody can be human, humanized, chimeric and/or affinity matured, as well as an antibody from other species, for example, mouse, rabbit, llama, etc. Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived.

Non-limiting examples of functional fragments (e.g., antigen-binding fragments) include single-chain Fvs (scFv) (e.g., including monospecific, bispecific, etc.), Fab fragments, F(ab′) fragments, F(ab)2 fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

In certain embodiments, the antibody or antigen-binding fragment is an IgG1 antibody or antigen-binding fragment. In certain embodiments, the antibody or antigen-binding fragment is an IgG2 antibody or antigen-binding fragment. In some embodiments, the antibody or antigen-binding fragment is a human IgG1 antibody or antigen-binding fragment or a human IgG2 antibody or antigen-binding fragment. In some embodiments, the antibody or antigen-binding fragment comprises a kappa light chain constant region or a lambda light chain constant region. In some embodiments, the antibody or antigen-binding fragment comprises a human kappa light chain constant region or a human lambda light chain constant region.

In certain embodiments, a bridging moiety, for example, an antibody or antigen-binding fragment of an antibody, specifically binds to an AAV1 particle. In another specific embodiment, a bridging moiety, for example, an antibody or antigen-binding fragment of an antibody, specifically binds to an AAV2 particle. In another specific embodiment, a bridging moiety, for example, an antibody or antigen-binding fragment of an antibody, specifically binds to an AAV3 particle. In another specific embodiment bridging moiety, for example, an antibody or antigen-binding fragment of an antibody, specifically binds to an AAV3B particle. In another specific embodiment, a bridging moiety, for example, an antibody or antigen-binding fragment of an antibody, specifically binds to an AAV4 particle. In another specific embodiment, a bridging moiety, for example, an antibody or antigen-binding fragment of an antibody, specifically binds to an AAV5 particle. In another specific embodiment, a bridging moiety, for example, an antibody or antigen-binding fragment of an antibody, specifically binds to an AAV6 particle. In another specific embodiment, a bridging moiety, for example, an antibody or antigen-binding fragment of an antibody, specifically binds to an AAV7 particle. In another specific embodiment, the bridging moiety is, for example, an antibody or antigen-binding fragment of an antibody, specifically binds to an AAV8 particle. In another specific embodiment, a bridging moiety, for example, an antibody or antigen-binding fragment of an antibody, specifically binds to an AAV9 particle. In another specific embodiment, a bridging moiety, for example, an antibody or antigen-binding fragment of an antibody, specifically binds to an AAV10 particle. In another specific embodiment, a bridging moiety, for example, an antibody or antigen-binding fragment of an antibody, specifically binds to an AAV11 particle. In another specific embodiment, a bridging moiety, for example, an antibody or antigen-binding fragment of an antibody, specifically binds to an AAV12 particle. In another specific embodiment, a bridging moiety, for example, an antibody or antigen-binding fragment of an antibody, specifically binds to an AAV13 particle. In another specific embodiment, a bridging moiety, for example, an antibody or antigen-binding fragment of an antibody, specifically binds to an AAV LK03 particle. In another specific embodiment, a bridging moiety, for example, an antibody or antigen-binding fragment of an antibody, specifically binds to an AAV rh10 particle. In another specific embodiment, a bridging moiety, for example, an antibody or antigen-binding fragment of an antibody, specifically binds to an AAV rh74 particle.

In certain embodiments, a bridging moiety, for example, an antibody or antigen-binding fragment of an antibody, specifically binds to an AAV particle of more than one AAV serotype. In specific embodiments, a bridging moiety, for example, an antibody or antigen-binding fragment of an antibody, specifically binds to an AAV particle of 2, 3, 4 or more AAV serotypes. In specific embodiments, a bridging moiety, for example, an antibody or antigen-binding fragment of an antibody, is an anti-AAV antibody that specifically binds multiple AAV serotypes. See, e.g., the anti-AAV VP1/VP2/VP3 monoclonal antibody at Progen Catalogue Number 61058 or LSBio Catalogue Number LS-C84096.

In certain embodiments, a bridging moiety, for example, an antibody or antigen-binding fragment of an antibody, does not comprise a Fc domain. In certain embodiments, a bridging moiety, for example, an antibody or antigen-binding fragment of an antibody, is a single-chain Fv (scFv).

In certain embodiments of the conjugates described herein, when L is bonded through an amide bond to a lysine residue of P, m is an integer from 1 to 80. In certain embodiments of the conjugates described herein, when L is bonded through a thioether bond to a cysteine residue of P, m is an integer from 1 to 8.

In certain embodiments, conjugation to the bridging moiety, e.g., antibody Ab, may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the bridging moiety, for example, antibody, e.g., on the heavy and/or light chains of an antibody that provide reactive thiol groups and do not disrupt polypeptide or antibody folding and assembly or alter polypeptide or antigen binding (see. e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009: 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety. All such methodologies are contemplated for use in connection with making the bridging compositions described herein.

In certain embodiments, the DAR for a bridging composition provided herein ranges from 1 to 80. In certain embodiments, the DAR ranges from 1 to 70. In certain embodiments, the DAR ranges from 1 to 60. In certain embodiments, the DAR ranges from 1 to 50. In certain embodiments, the DAR ranges from 1 to 40. In certain embodiments, the DAR ranges from 1 to 35. In certain embodiments, the DAR ranges from 1 to 30. In certain embodiments, the DAR ranges from 1 to 25. In certain embodiments, the DAR ranges from 1 to 20. In certain embodiments, the DAR ranges from 1 to 18. In certain embodiments, the DAR ranges from 1 to 15. In certain embodiments, the DAR ranges from 1 to 12. In certain embodiments, the DAR ranges from 1 to 10. In certain embodiments, the DAR ranges from 1 to 9. In certain embodiments, the DAR ranges from 1 to 8. In certain embodiments, the DAR ranges from 1 to 7. In certain embodiments, the DAR ranges from 1 to 6. In certain embodiments, the DAR ranges from 1 to 5. In certain embodiments, the DAR ranges from 1 to 4. In certain embodiments, the DAR ranges from 1 to 3. In certain embodiments, the DAR from 2 to 12. In certain embodiments, the DAR from 2 to 10. In certain embodiments, the DAR ranges from 2 to 9. In certain embodiments, the DAR ranges from 2 to 8. In certain embodiments, the DAR ranges from 2 to 7. In certain embodiments, the DAR ranges from 2 to 6. In certain embodiments, the DAR ranges from 2 to 5. In certain embodiments, the DAR ranges from 2 to 4. In certain embodiments, the DAR ranges from 3 to 12. In certain embodiments, the DAR ranges from 3 to 10. In certain embodiments, the DAR ranges from 3 to 9. In certain embodiments, the DAR ranges from 3 to 8. In certain embodiments, the DAR ranges from 3 to 7. In certain embodiments, the DAR ranges from 3 to 6. In certain embodiments, the DAR ranges from 3 to 5. In certain embodiments, the DAR ranges from 3 to 4.

In certain embodiments, the DAR for a bridging composition provided herein provided herein ranges from 1 to about 8; from about 2 to about 6; from about 3 to about 5: from about 3 to about 4; from about 3.1 to about 3.9; from about 3.2 to about 3.8; from about 3.2 to about 3.7: from about 3.2 to about 3.6; from about 3.3 to about 3.8; or from about 3.3 to about 3.7.

In certain embodiments, the DAR for a bridging composition provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9.

In some embodiments, the DAR for a bridging composition provided herein ranges from 2 to 20, 2 to 19, 2 to 18, 2 to 17, 2 to 16, 2 to 15, 2 to 14, or 2 to 13. In some embodiments, the DAR ranges from 3 to 20, 3 to 19, 3 to 18, 3 to 17, 3 to 16, 3 to 15, 3 to 14, or 3 to 13. In some embodiments, the DAR is about 1. In some embodiments, the DAR is about 2. In some embodiments, the DAR is about 3. In some embodiments, the DAR is about 4. In some embodiments, the DAR is about 3.8. In some embodiments, the DAR is about 5. In some embodiments, the DAR is about 6. In some embodiments, the DAR is about 7. In some embodiments, the DAR is about 8. In some embodiments, the DAR is about 9. In some embodiments, the DAR is about 10. In some embodiments, the DAR is about 11. In some embodiments, the DAR is about 12. In some embodiments, the DAR is about 13. In some embodiments, the DAR is about 14. In some embodiments, the DAR for a conjugate provided herein is about 15. In some embodiments, the DAR is about 16. In some embodiments, the DAR is about 17. In some embodiments, the DAR is about 18. In some embodiments, the DAR is about 19. In some embodiments, the DAR is about 20.

In some embodiments, the DAR for a bridging composition provided herein is about 25. In some embodiments, the DAR is about 30. In some embodiments, the DAR is about 35. In some embodiments, the DAR is about 40. In some embodiments, the DAR is about 50. In some embodiments, the DAR is about 60. In some embodiments, the DAR is about 70. In some embodiments, the DAR is about 80.

In certain embodiments, m is an integer from 1 to 80. In certain embodiments, m is an integer from 1 to 8. In certain embodiments, m is an integer from 4 to 8. In certain embodiments, m is 4. In certain embodiments, m is 3. In certain embodiments, m is 2. In certain embodiments, m is 1.

In certain embodiments, fewer than the theoretical maximum of units are conjugated to the bridging moiety, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a bridging composition may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the bridging moiety, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a bridging moiety, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

5.2.3. Linking Moieties

5.2.3.1 Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a component of a bridging moiety and the other for conjugation to a moiety (noted as X) that binds to a cell surface receptor. For example, if the cell surface receptor is a mannose-6-phosphate receptor (M6PR), then the moiety may be mannose-6-phosphate or an analog of a mannose-6-phosphate moiety. When the component of a viral composition Y comprises a polypeptide, the polypeptide conjugation reactive terminus of the linker is in some cases a site that is capable of conjugation to the polypeptide through a cysteine thiol or lysine amine group on the polypeptide, and so it can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In some embodiments of formula (Ia), the linker L comprises one or more straight or branched-chain carbon moieties and polyether (e.g., PEG) moieties, and combinations thereof. In certain embodiments, these linkers optionally have amide linkages, sulfhydryl linkages, or hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments. L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 400 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 200 Å, which comprises an optionally substituted arylene linked to X, optionally substituted heteroarylene linked to X, optionally substituted heterocyclene linked to X, or optionally substituted cycloalkylene linked to X.

In certain embodiments, linker L separates X and Y (or Z) by a chain of 4 to 500 consecutive atoms. In certain embodiments, linker L separates X and Y (or Z) by a chain of 4 to 50 consecutive atoms. In certain embodiments, linker L separates X and Y (or Z) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z) by a chain of 6 to 50 consecutive atoms. In certain embodiments, linker L separates X and Y (or Z) by a chain of 11 to 50 consecutive atoms. In certain embodiments, linker L separates X and Y (or Z) by a chain of 16 to 50 consecutive atoms. In certain embodiments, linker L separates X and Y (or Z) by a chain of 21 to 50 consecutive atoms. In certain embodiments, linker L separates X and Y (or Z) by a chain of 26 to 50 consecutive atoms. In certain embodiments, linker L separates X and Y (or Z) by a chain of 31 to 50 consecutive atoms. In certain embodiments, linker L separates X and Y (or Z) by a chain of 36 to 50 consecutive atoms. In certain embodiments, linker L separates X and Y (or Z) by a chain of 41 to 50 consecutive atoms. In certain embodiments, linker L separates X and Y (or Z) by a chain of 46 to 50 consecutive atoms.

In certain embodiments, linker L separates X and Y (or Z) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L is a chain of 5 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 7 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 10 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 15 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

In certain embodiments, linker L is a chain of 5 to 500 consecutive atoms separating X and Y (or Z) and which comprises an optionally substituted arylene linked to X or optionally substituted heteroarylene linked to X. In certain embodiments, linker L is a chain of 7 to 500 consecutive atoms separating X and Y (or Z) and which comprises an optionally substituted arylene linked to X or optionally substituted heteroarylene linked to X. In certain embodiments, linker L is a chain of 10 to 500 consecutive atoms separating X and Y (or Z) and which comprises an optionally substituted arylene linked to X or optionally substituted heteroarylene linked to X. In certain embodiments, linker L is a chain of 15 to 400 consecutive atoms separating X and Y (or Z) and which comprises an optionally substituted arylene linked to X or optionally substituted heteroarylene linked to X.

In certain embodiments, linker L is a chain of 5 to 500 consecutive atoms separating X and Y (or Z) and which comprises an optionally substituted phenylene linked to X. In certain embodiments, linker L is a chain of 7 to 500 consecutive atoms separating X and Y (or Z) and which comprises an optionally substituted phenylene linked to X. In certain embodiments, linker L is a chain of 10 to 500 consecutive atoms separating X and Y (or Z) and which comprises an optionally substituted phenylene linked to X. In certain embodiments, linker L is a chain of 15 to 400 consecutive atoms separating X and Y (or Z) and which comprises an optionally phenylene linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

It is understood that the linker may be considered as connecting directly to a Z² group of a M6PR binding moiety (X) (e.g., as described herein). In some embodiments of formula (XI), the linker may be considered as connecting directly to the Z³ group. Alternatively, the —Ar—Z³— group of formula (XI) (e.g., as described herein) can be considered part of a linking moiety that connects Z² to Y. The disclosure is meant to include all such configurations of M6PR binding moiety (X) and linker (L).

In some embodiments of formula (Ia), L is a linker of the following formula (IIa):

-[(L¹)ₐ-(L²)_{b}-(L³)_{c}]_{n-(L}⁴)_{d}-(L⁵)ₑ-(L⁶)_{f}-(L⁷)_{g}- (IIa)

wherein:

each L¹ to L⁷ is independently a linking moiety:

a is 1 or 2;

b, c, d, e, f, and g are each independently 0, 1, or 2; and

n is 1 to 500.

In some embodiments of formula (IIa), n is an integer of 1 to 5; wherein when d is 0, n is 1, when d is 1, n is an integer of 1 to 3, and when d is 2, n is an integer of 1 to 5.

In some embodiments of formula (IIa), L¹ comprises an optionally substituted aryl or heteroaryl group or linking moiety, e.g., as described in formula (XI). In some embodiments of formula (IIa), L¹ comprises a monocyclic or bicyclic or tricyclic aryl or heteroaryl group that is optionally substituted (e.g., as described herein). In some embodiments of formula (IIa), L¹ further comprises one or more linking moieties, each independently selected from a C₍₁₋₁₀₎alkyl, —O—, —S—, —NH—, —NHCO—, —CONH—, —NHC(═O)NH—, —NHC(═S)NH—, —NHCO₂—, —OC(═O)NH—, —OC(═O)—, —CO₂—, —(OCH₂)ₚ—, and —(OCH₂CH₂)ₚ—, where p is 1-20, such as 1-10, 1-6 or 1-3, e.g., 1 or 2.

In some embodiments of formula (IIa), each L¹ is independently

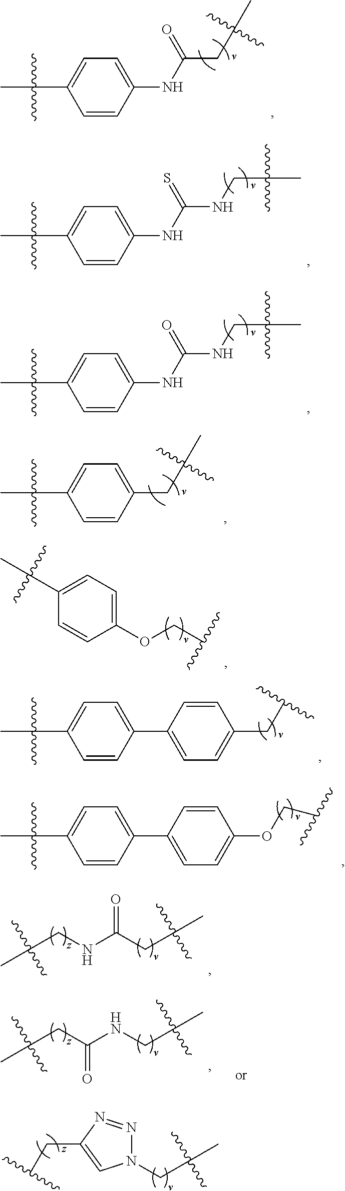

where z and v are independently 0-10, such as 0-6 or 0-3, e.g., 0, 1 or 2.

In certain embodiments of formula (IIa), L¹ is

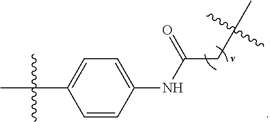

In certain embodiments of formula (IIa), L¹ is

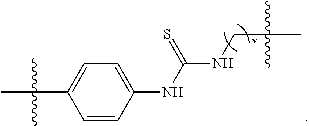

In certain embodiments of formula (IIa), L¹ is

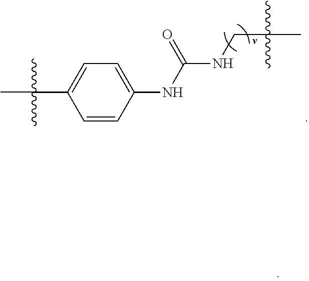

In certain embodiments of formula (IIa), L¹ is

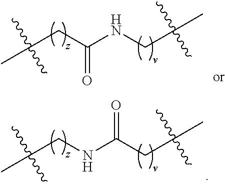

In certain embodiments of formula (IIa), L¹ is

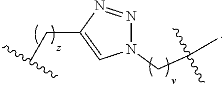

In certain embodiments of formula (IIa), each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—, where p is 1-20, such as 1-10, 1-6 or 1-3, e.g., 1 or 2.

In certain embodiments of formula (IIa), each L³ is independently

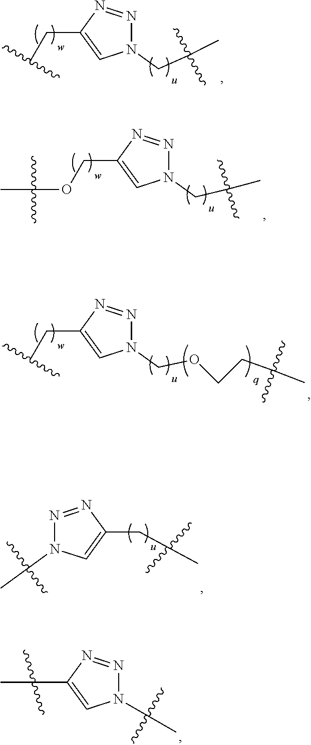

or —(OCH₂CH₂)_{q}—, where w and u are independently 0-10, such as 1-10, 1-6 or 1-3, e.g., 1 or 2, and q is 1-20 such as 1-10, 1-6 or 1-3, e.g., 1 or 2.

In some embodiments of formula (IIa), each L⁴ is a linear or branched linking moiety.

In some embodiments of formula (IIa), L⁴ is a branched linking moiety, e.g., a trivalent linking moiety. For example, an L⁴ linking moiety can be of the one of the following general formula:

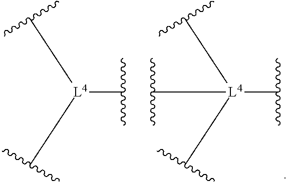

In some embodiments of formula (IIa), the branched linking moiety can be of higher valency and be described by one of the one of the following general formula:

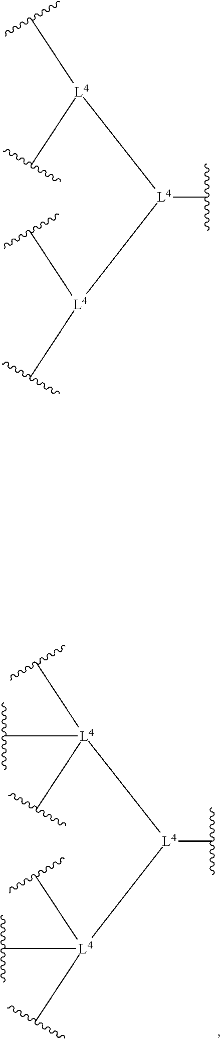

where any two L⁴ groups can be directed linked or connected via optional linear linking moieties (e.g., as described herein).

In some embodiments of formula (IIa), the branched linking moiety can include one, two or more L4 linking moieties, each being trivalent moieties, which when linked together can provide for multiple branching points for covalent attachment of the ligands and be described by one of the one of the following general formula:

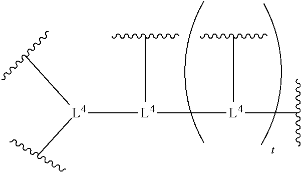

where t is 0 to 500, such as 0 to 100, 0 to 20, or 0 to 10.

In some embodiments, the branched linking moiety (e.g., L⁴) comprises one or more of: an amino acid residue (e.g., Asp, Lys, Orn, Glu), N-substituted amido (—N(—)C(═O)—), tertiary amino, polyol (e.g., O-substituted glycerol), and the like.

In some embodiments of formula (IIa), one or more L⁴ is selected from

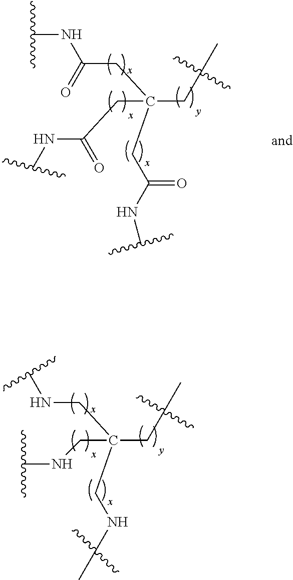

wherein each x and y is independently 1 to 20. In some cases, each x is 1, 2 or 3, e.g., 2.

In some embodiments of formula (IIa), each L⁴ is independently —OCH₂CH₂—,

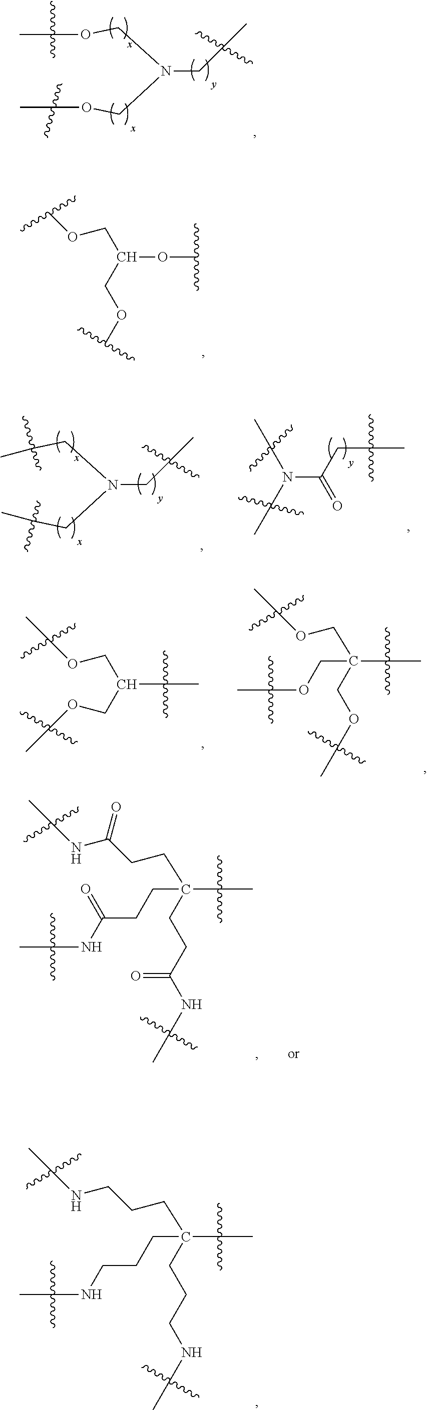

where each x and y are independently 1-10, such as 1-6 or 1-3, e.g., 1 or 2.

In some embodiments of formula (IIa), each L⁵ is independently —NHCO—C₁₋₆-alkylene-, —CONH—C₁₋₆-alkylene-, —C₁₋₆-alkylene-,

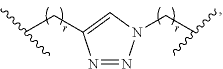

or —(OCH₂CH₂)ᵣ—, where each r is independently 1-20, such as 1-10, 1-6 or 1-3, e.g., 1 or 2.

In some embodiments of formula (IIa), each L⁶ is independently —NHCO—C₁₋₆-alkylene-, —CONH—C₁₋₆-alkylene-, —C₁₋₆alkylene-, or —(OCH₂CH₂)ₛ—, where s is 1-20, such as 1-10, 1-6 or 1-3, e.g., 1 or 2.

In some embodiments of formula (IIa), each L⁷ is independently —NHCO—C₁₋₆-alkylene-, —CONH—C₁₋₆-alkylene-, —C₁₋₆-alkylene-, —(OCH₂CH₂)ₜ—, or —OCH₂—, where t is 1-20, such as 1-10, 1-6 or 1-3, e.g., 1 or 2.

In certain embodiments of formula (IIa), a is 1. In certain embodiments of formula (IIa), a is 1, and b, c, d, c, f, and g are 0.

In certain embodiments of formula (IIa), at least one of b, c, e, f, and g is not 0. In certain embodiments of formula (IIa), a, b, c and d are 1 and e, f and g are 0. In certain embodiments of formula (IIa), a, b, c, d and g are 1 and e and f are 0. In certain embodiments of formula (IIa), a, b, d, e and f are 1; c and g are 0; z is an integer from 2 to 10 and n is an integer of 1 to 5.

In certain embodiments of formula (IIa), at least one of b or c is not 0 and at least one of e, f, and g is not 0. In certain embodiments of formula (IIa), a, b, c, d, e and f are 1 and g is 0 or 1. In certain embodiments of formula (IIa), a, b, c, d, e, f and g are 1.

In certain embodiments of formula (IIa), a, b, and c are each independently 1 or 2.

In certain embodiments, k, p, q, r, s, and t are each independently an integer of 1 to 20. In certain embodiments, k, p, q, r, s, and t are each independently an integer of 1 to 10. In certain embodiments, k, p, q, r, s, and t are each independently an integer of 1 to 5. In certain embodiments, k, p, q, r, s, and t are each independently an integer of 1 to 3.

In certain embodiments, p, q, r, s, and t are each independently an integer of 1 to 20. In certain embodiments, p, q, r, s, and t are each independently an integer of 1 to 10. In certain embodiments, p, q, r, s, and t are each independently an integer of 1 to 5. In certain embodiments, p, q, r, s, and t are each independently an integer of 1 to 3.

In certain embodiments, u, v, w, x, y, and z are each independently an integer of 1 to 10. In certain embodiments, u, v, w, x, y, and z are each independently an integer of 1 to 5. In certain embodiments, u, v, w, x, y, and z are each independently an integer of 1 to 3.

In certain embodiments of formula (IIa), n is 1. In certain embodiments of formula (IIa), n is 2. In certain embodiments of formula (IIa), n is 3. In certain embodiments of formula (IIa), n is 4. In certain embodiments of formula (IIa), n is 5.

Tables 2-3 shows a variety of exemplary linkers or linking moieties that find use in the modified viral compositions described herein. In some embodiments of formula (I)-(IIe) or (XI)-(XVIa), the compound includes any one of the linkers or linking moieties set forth in Tables 2-3.

TABLE 4
Exemplary linear linkers and linking moieties

| Linker | |
|---|---|
| No. | Linker structure |
| 1 | 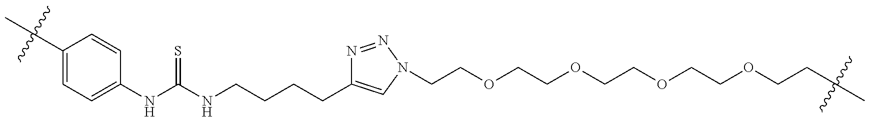 [SMILES: *CCOCCOCCOCCOCCn1cc(CCCCNC(=S)Nc2ccc(*)cc2)nn1] |
| 2 | 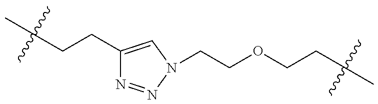 [SMILES: *CCOCCn1cc(CC*)nn1] |
| 3 | 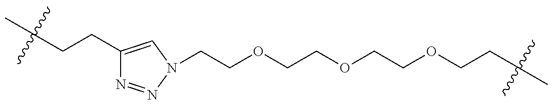 [SMILES: *CCOCCOCCOCCn1cc(CC*)nn1] |
| 4 | 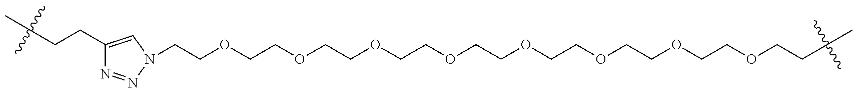 [SMILES: *CCOCCOCCOCCOCCOCCOCCOCCOCCn1cc(CC*)nn1] |
| 5 | 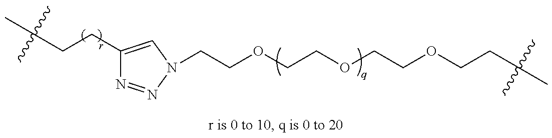 |
| 6 | 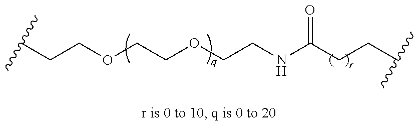 |
| 7 | 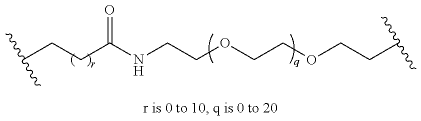 |
| 8 | 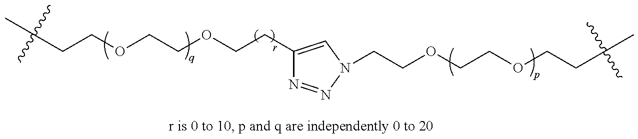 |
| 9 | 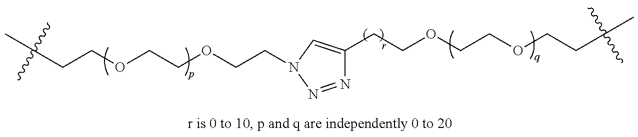 |
| 10 | 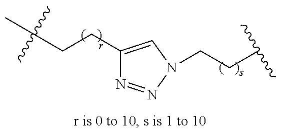 |

TABLE 5
Exemplary branched linkers and branched linking moieties

| Linker | |
|---|---|
| No. | Linker structure |
| 11 | 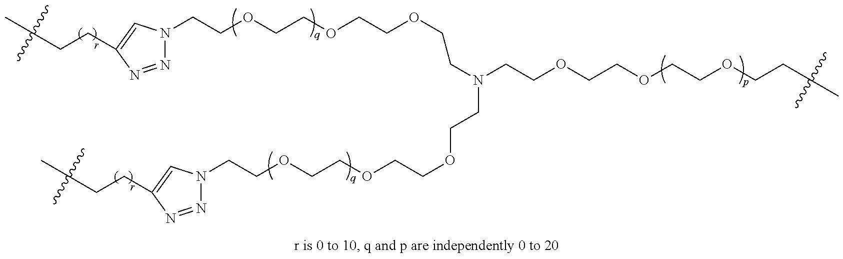 |
| 12 | 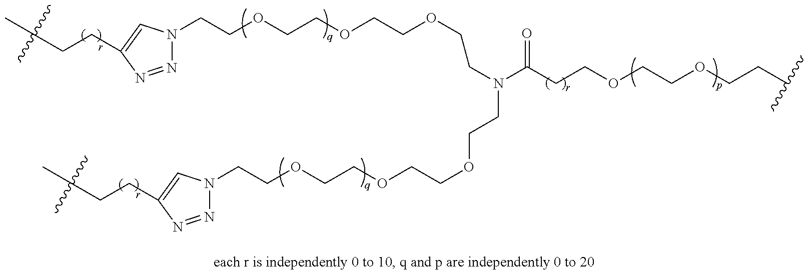 |
| 13 | 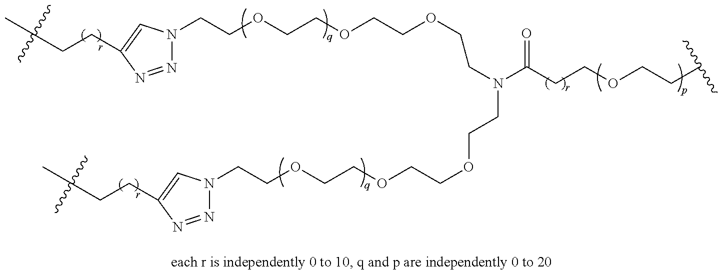 |
| 14 | 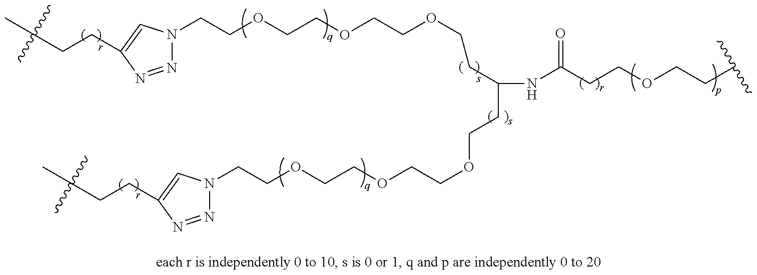 |
| 15 | 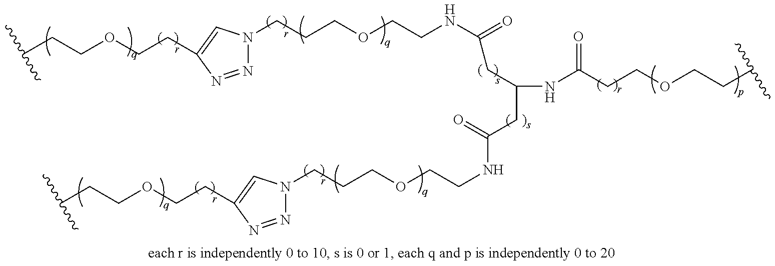 |
| 16 | 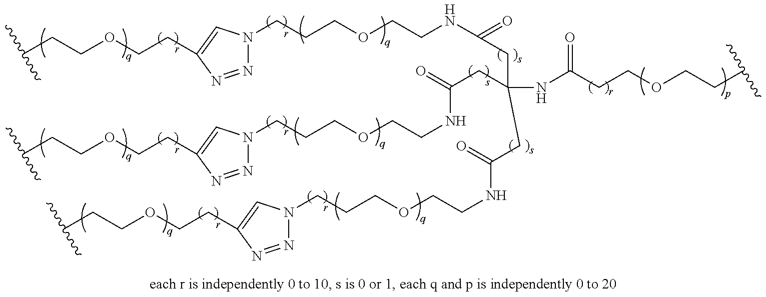 |
| 17 | 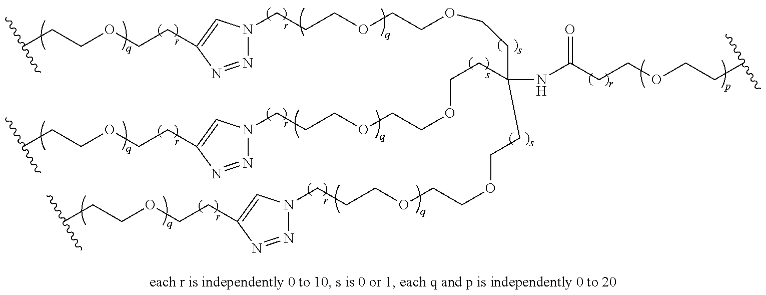 |

As summarized above, the cell surface receptor binding compounds that find use in preparing the bridging compositions and modified viral compositions of this disclosure, e.g., compositions including a bridging moiety conjugate, generally include, or derive from, a chemoselective ligation group capable of conjugation to a compatible reactive group of another moiety of interest, e.g., a bridging moiety as described herein.

5.2.3.2 Chemoselective Ligation Groups

A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, reductive amination, dialkyl squarate chemistry, etc.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

Conjugates of the bridging moiety and binding moiety for cell surface receptor may be made using a variety of linkers and/or bifunctional protein coupling agents such as BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances of formula (Ia)-(XVIa), Y is a precursor of the reactive functionality or function group capable of conjugation to a compatible group of a second moiety. For example, a carboxylic acid is a precursor of an active ester chemoselective ligation group.

In certain embodiments of formula (Ia)-(XVIa), Y is a reactive moiety capable forming a covalent bond to a polypeptide (e.g., with an amino acid sidechain of a polypeptide having a compatible reactive group). The reactive moiety can be referred to as a chemoselective ligation group.

In certain embodiments of formula (Ia)-(XVIa), Y is a thio-reactive chemoselective ligation group (e.g., as described in Table 6). In some cases, Y can produce a residual moiety Z resulting from the covalent linkage of a thiol-reactive chemoselective ligation group to one or more cysteine residue(s) of a protein, e.g., Ab.

In certain embodiments of formula (Ia)-(XVIa), Y is an amino-reactive chemoselective ligation group (e.g., as described in Table 6). In some cases, Y can produce a residual moiety Z resulting from the covalent linkage of an amine-reactive chemoselective ligation group to one or more lysine residue(s) a protein, e.g., of a viral composition.

In certain embodiments of the bridging compositions, L is bonded through an amide bond to a lysine residue of P. In certain embodiments of the conjugates described herein. L is bonded through a thioether bond to a cysteine residue of P. In certain embodiments of the conjugates described herein, L is bonded through an amide bond to a lysine residue of Ab, as depicted above. In certain embodiments of the conjugates described herein, L is bonded through a thioether bond to a cysteine residue of Ab, as depicted above. In certain embodiments of the conjugates described herein, L is bonded through two thioether bonds to two cysteine residues of Ab, wherein the two cysteine residues are from an opened cysteine-cysteine disulfide bond in Ab, as depicted above. In certain embodiments, the opened cysteine-cysteine disulfide bond is an interchain disulfide bond.

Exemplary chemoselective ligation groups, and synthetic precursors thereof, which may be adapted for use in preparing the conjugates of this disclosure, e.g., including a bridging moiety, are shown in Table 6.

TABLE 6
Exemplary chemoselective ligation groups and precursors

| Groups | Exemplary structures |
|---|---|
| carboxylic acid or active ester | 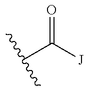 |
| | where J is selected from —OH, —Cl, —Br, —I, —F, —OH, —O—N-succinimide, —O- |
| | (4-nitrophenyl), —O-pentafluorophenyl, —O-tetrafluorophenyl, and |
| | —O—C(O)—Or^{J′}, and R^{J′} is -C₁-C₈ alkyl or -aryl, |
| | 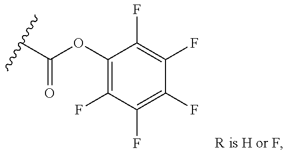 [SMILES: *C(=O)Oc1c(F)c(F)c(F)c(F)c1F] |
| | 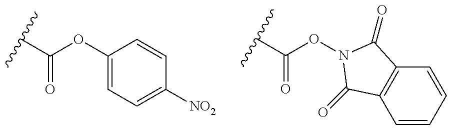 [SMILES: *C(=O)Oc1ccc([N+](=O)[O-])cc1 ; *C(=O)ON1C(=O)c2ccccc2C1=O] |
| | 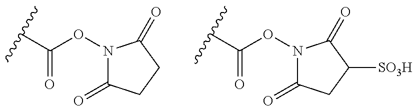 [SMILES: *C(=O)ON1C(=O)CCC1=O ; *C(=O)ON1C(=O)CC(S(=O)(=O)O)C1=O] |
| | 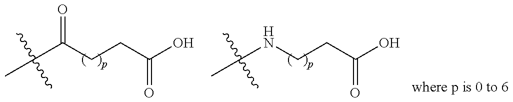 |
| maleimide | 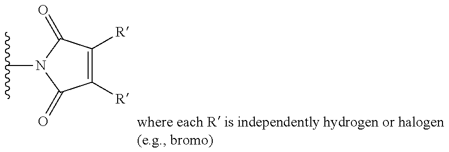 |
| isocyanate or | —NCS |
| isothiocyanate | —NCO |
| | 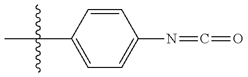 [SMILES: *c1ccc(N=C=O)cc1] |
| | 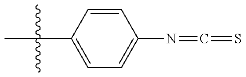 [SMILES: *c1ccc(N=C=S)cc1] |
| alkyl halide | 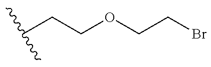 [SMILES: *CCOCCBr] |
| alkyl tosylate | 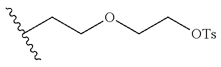 [SMILES: *CCOCCOS(=O)(=O)c1ccc(C)cc1] |
| aldehyde | 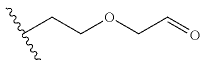 [SMILES: *CCOCC=O] |
| haloacetamide or alpha-leaving group acetamide | 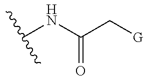 |
| | where G is selected from —F, —Cl, —Br, —I, —O-mesyl, and —O-tosyl |
| 2-sulfonylpyridine | 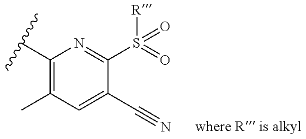 |
| diazirine | 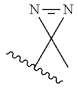 [SMILES: *C1(C)N=N1] |
| sulfonyl halide or vinyl sulfone | 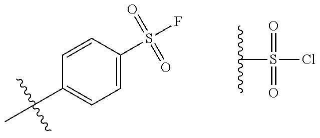 [SMILES: *c1ccc(S(=O)(=O)F)cc1 ; *S(=O)(=O)Cl] |
| | 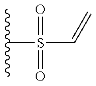 [SMILES: *S(=O)(=O)C=C] |
| hydrazide hydrazino hydroxylamino | 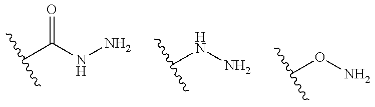 |
| pyridyl disulfide | 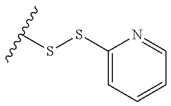 [SMILES: *SSc1ccccn1] |
| (HIPS) hydrazinyl- indolyl group, or (aza-HIPS) hydrazinyl- pyrrolo-pyridinyl group | 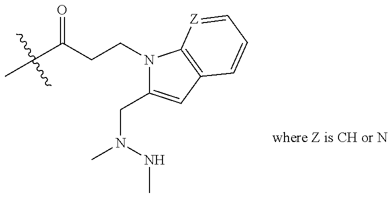 |
| alkyne or cyclooctyne | 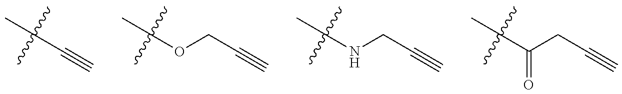 |
| | 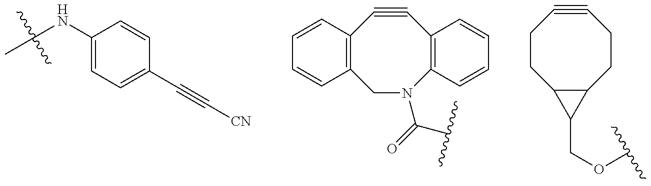 |
| azide | 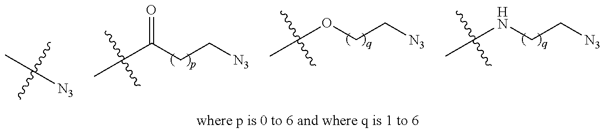 |
| amine | 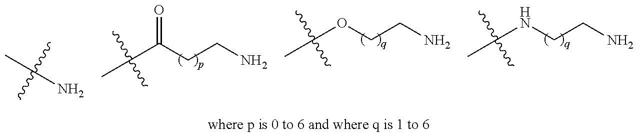 |

In Table 6, the [symbol] can represent a point of attachment of Y to a linking moiety or a linked X moiety.

Conjugation of a chemoselective ligation group of a compounds of formula (Ta) with a bridging moiety produces a conjugate (e.g., of formula (I) that includes a residual group produced during conjugation, e.g., group Z of formula (I)-(Ib). It is understood that particular residue Z groups are produced in the conjugates of this disclosure from the reaction of compatible chemoselective ligation groups. Exemplary residual Z groups, e.g., of formula (I) and (Ib) are shown below:

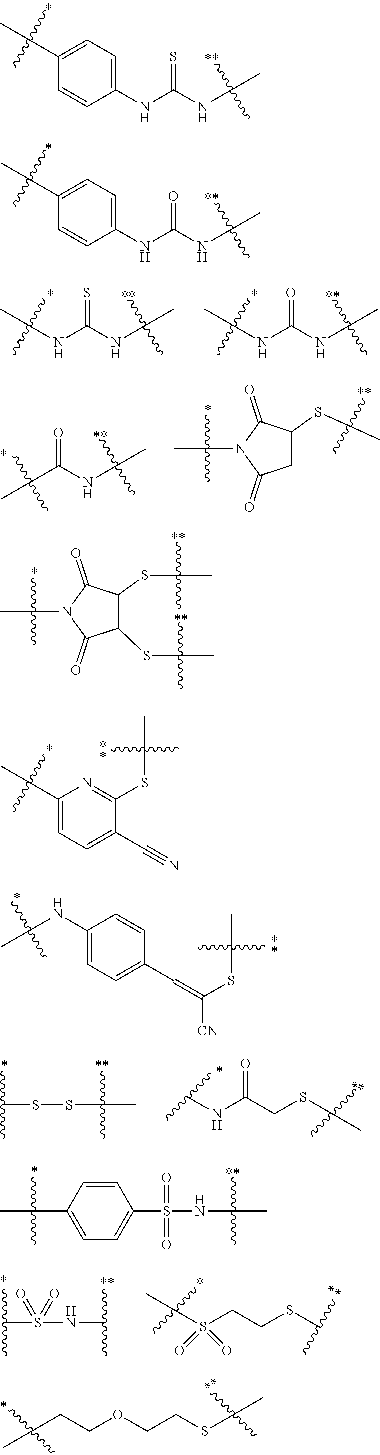

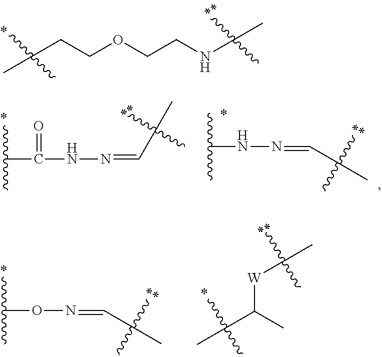

where W is CH₂, N, O or S.

In some embodiments, [symbol] represents the point of attachment to linker L or X, and [symbol] represents the point of attachment to bridging moiety P.

5.2.4. Exemplary Compounds with Chemoselective Ligation Group

This disclosure includes bridging compositions which can include: (1) one or more particular M6PR ligand (X) (e.g., as described herein, such as ligands X1-X38 of Table 1) or a particular ASGPR ligand (X) (e.g., as described herein) or a particular folate receptor ligand (X) (e.g., as described herein), (2) a linker including one or more linking moieties (e.g., as described herein, such as any one or more of the linking moieties of Tables 4-5); and (3) a chemoselective ligation group (Y) e.g., as described herein, such as any one of the groups of Table 6) that has been conjugated to a bridging moiety.

Tables 7-10 illustrate several exemplary M6PR binding compounds of this disclosure that include a chemoselective ligation group, or a precursor thereof. It is understood that this disclosure includes Y (e.g., bridging moiety, as described herein) conjugates of each of the exemplary compounds of Tables 7-10.

Tables 11-12 illustrate several exemplary ASGPR binding compounds of this disclosure that include a chemoselective ligation group, or a precursor thereof. It is understood that this disclosure includes Y conjugates (e.g., with a bridging moiety, as described herein) of each of the exemplary compounds of Tables 8-9.

The experimental section illustrates several exemplary folate receptor binding compounds of this disclosure that include a chemoselective ligation group, or a precursor thereof. It is understood that this disclosure includes Y (e.g., bridging moiety, as described herein) conjugates of each of the exemplary folate receptor binding compounds.

TABLE 7
Exemplary M6PR binding compounds of Formula (XIa)

| # | Compound structure |
|---|---|
| 501 (I-1) | 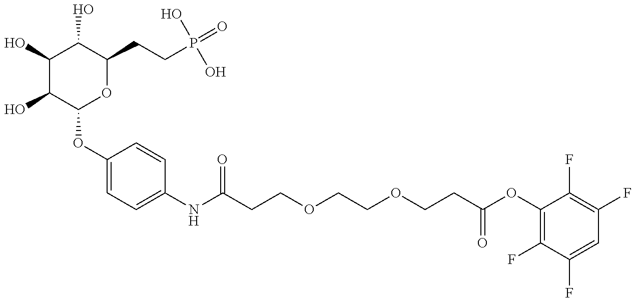 [SMILES: O=C(CCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 502 | 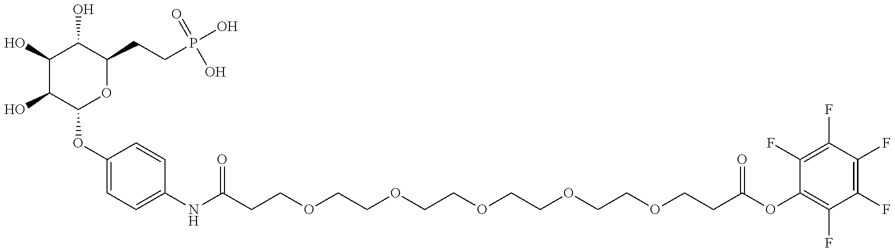 [SMILES: O=C(CCOCCOCCOCCOCCOCCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 503 | 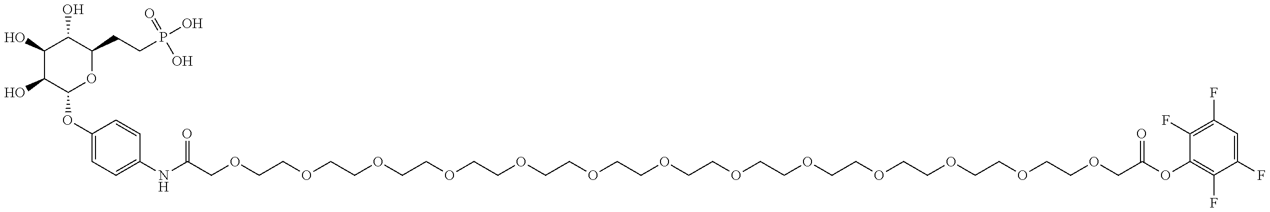 [SMILES: O=C(COCCOCCOCCOCCOCCOCCOCCOCCOCCOCCOCCOCCOCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 504 | 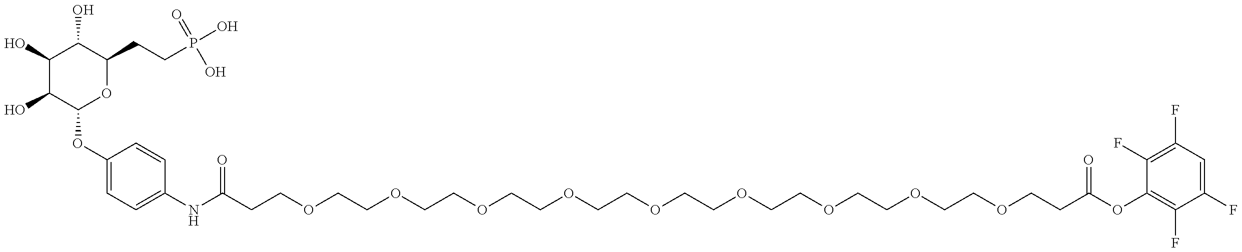 [SMILES: O=C(CCOCCOCCOCCOCCOCCOCCOCCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 505 (I-2) | 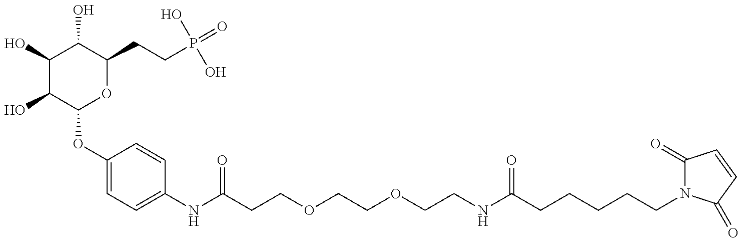 [SMILES: O=C(CCCCCN1C(=O)C=CC1=O)NCCOCCOCCC(=O)Nc1ccc(O[C@H]2O[C@H](CCP(=O)(O)O)[C@@H](O)[C@H](O)[C@@H]2O)cc1] |
| 506 | 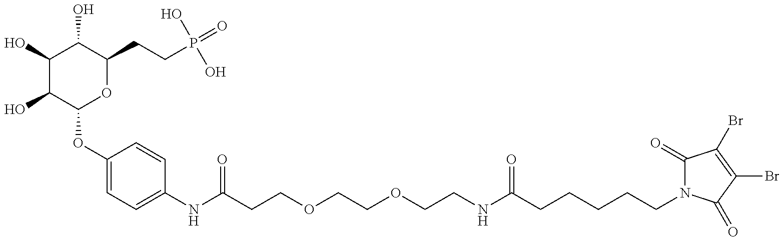 [SMILES: O=C(CCCCCN1C(=O)C(Br)=C(Br)C1=O)NCCOCCOCCC(=O)Nc1ccc(O[C@H]2O[C@H](CCP(=O)(O)O)[C@@H](O)[C@H](O)[C@@H]2O)cc1] |
| 507 | 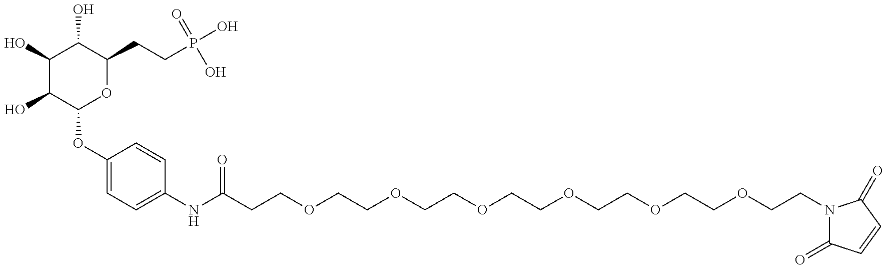 [SMILES: O=C(CCOCCOCCOCCOCCOCCOCCN1C(=O)C=CC1=O)Nc1ccc(O[C@H]2O[C@H](CCP(=O)(O)O)[C@@H](O)[C@H](O)[C@@H]2O)cc1] |
| 508 (I-3) | 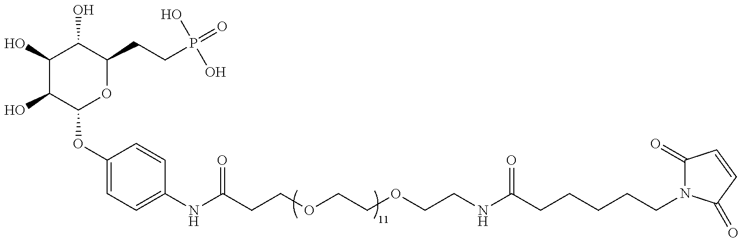 |
| 509 | 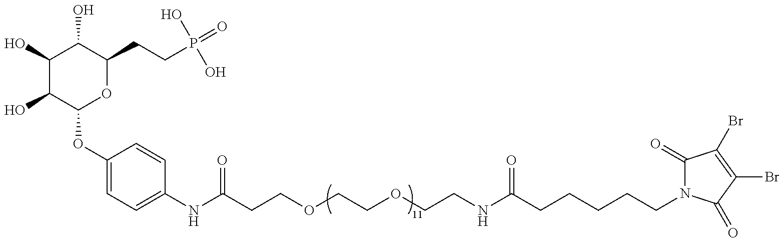 |
| 510 | 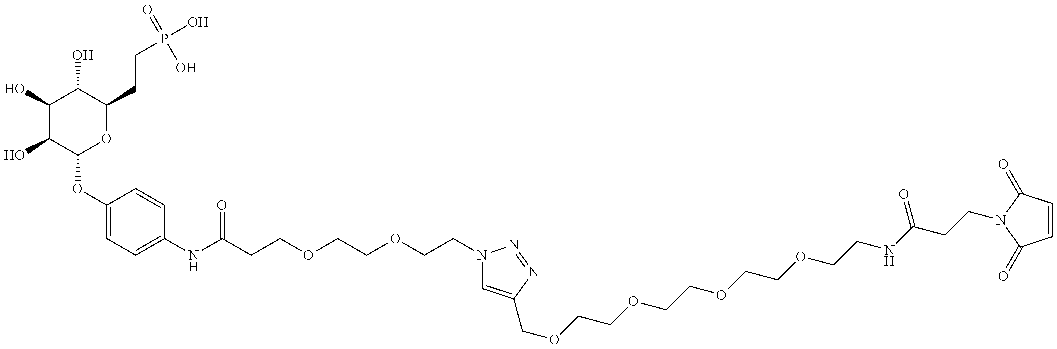 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 511 (I-4) | 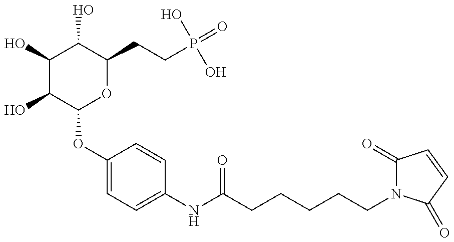 [SMILES: O=C(CCCCCN1C(=O)C=CC1=O)Nc1ccc(O[C@H]2O[C@H](CCP(=O)(O)O)[C@@H](O)[C@H](O)[C@@H]2O)cc1] |
| 512 (I-5) | 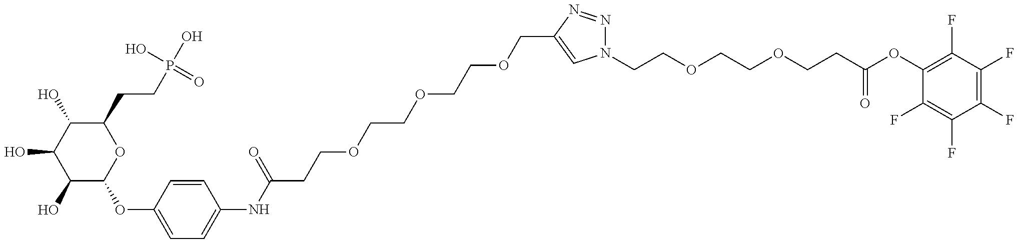 [SMILES: O=C(CCOCCOCCOCc1cn(CCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 513 (I-39) | 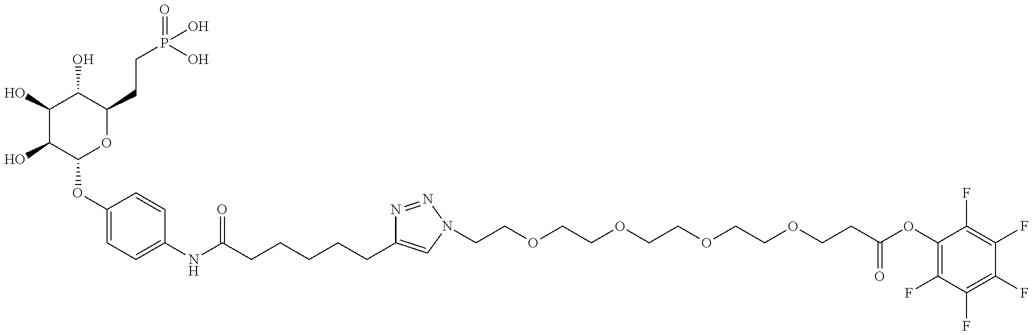 [SMILES: O=C(CCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 514 (I-57) | 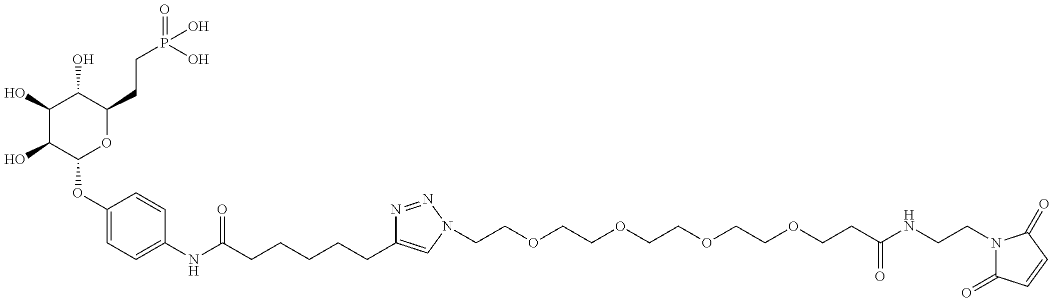 [SMILES: O=C(CCOCCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 515 (I-16) | 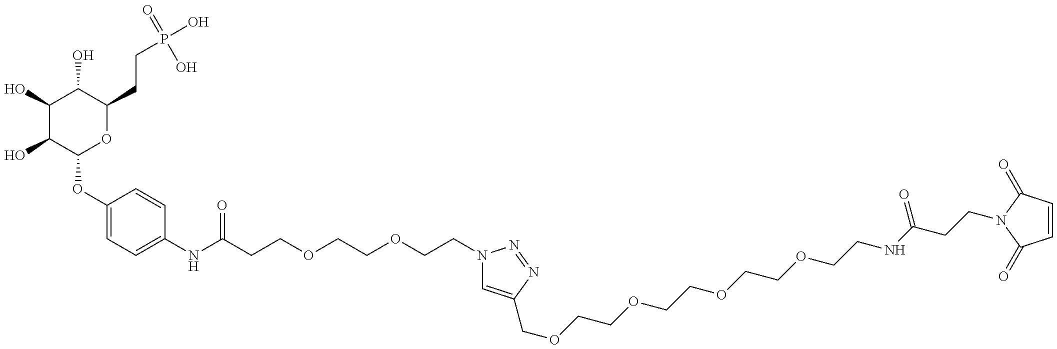 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 516 (I-6) | 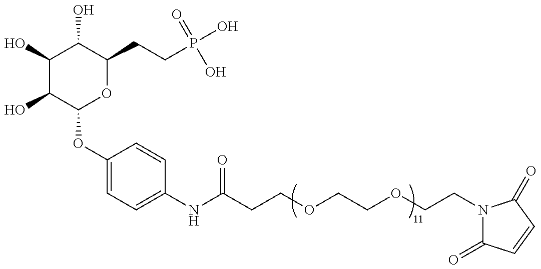 |
| 517 | 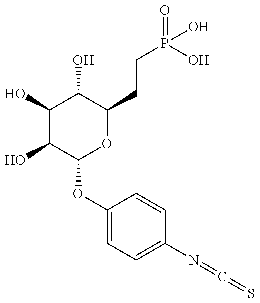 [SMILES: O=P(O)(O)CC[C@H]1O[C@H](Oc2ccc(N=C=S)cc2)[C@@H](O)[C@@H](O)[C@@H]1O] |
| 518 | 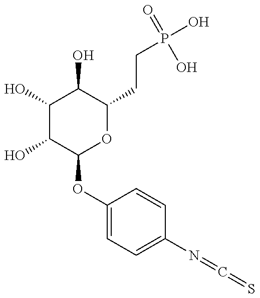 [SMILES: O=P(O)(O)CC[C@@H]1O[C@@H](Oc2ccc(N=C=S)cc2)[C@H](O)[C@H](O)[C@H]1O] |
| 519 (I-47) | 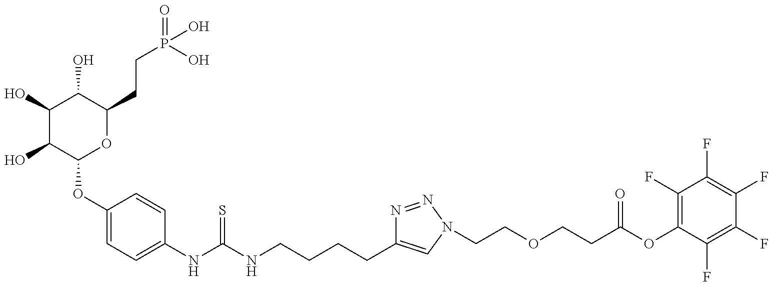 [SMILES: O=C(CCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 520 (I-7) | 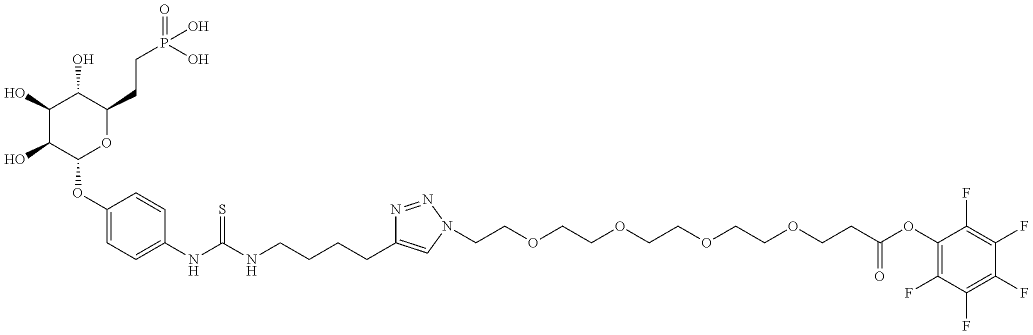 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 521 | 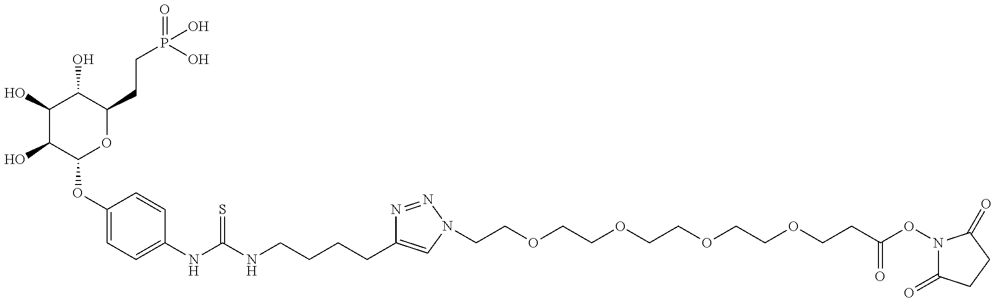 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)ON1C(=O)CCC1=O] |
| 522 (I-49) | 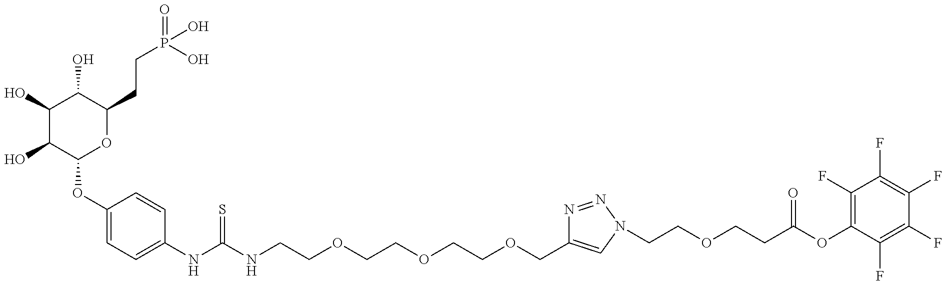 [SMILES: O=C(CCOCCn1cc(COCCOCCOCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 523 (I-17) | 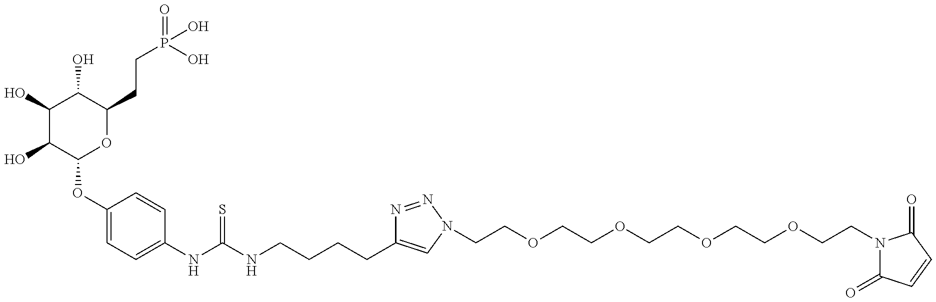 [SMILES: O=C1C=CC(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 524 (I-18) | 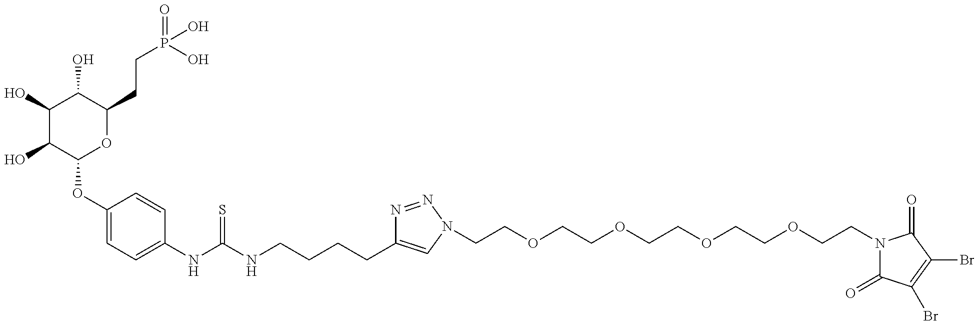 [SMILES: O=C1C(Br)=C(Br)C(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 525 | 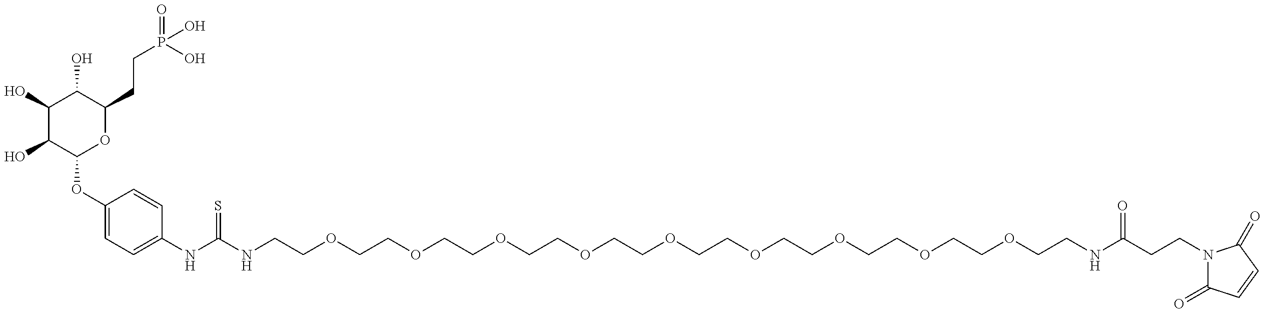 [SMILES: O=C(CCN1C(=O)C=CC1=O)NCCOCCOCCOCCOCCOCCOCCOCCOCCOCCNC(=S)Nc1ccc(O[C@H]2O[C@H](CCP(=O)(O)O)[C@@H](O)[C@H](O)[C@@H]2O)cc1] |
| 526 (I-48) | 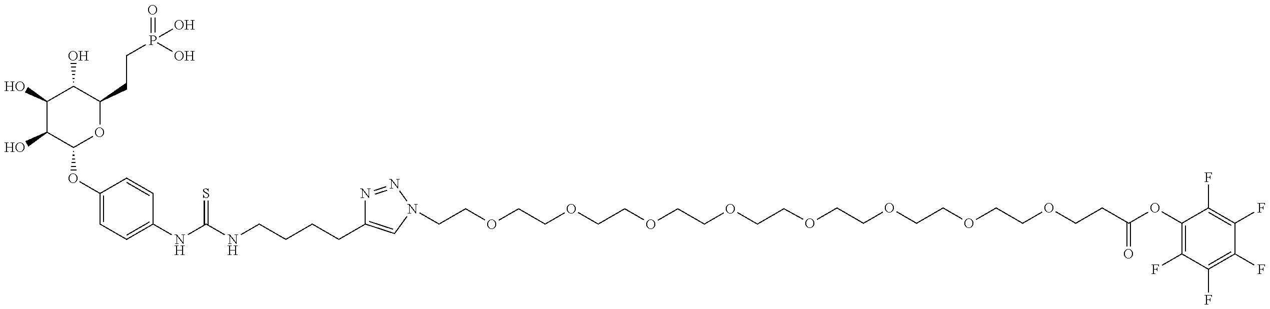 [SMILES: O=C(CCOCCOCCOCCOCCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 527 | 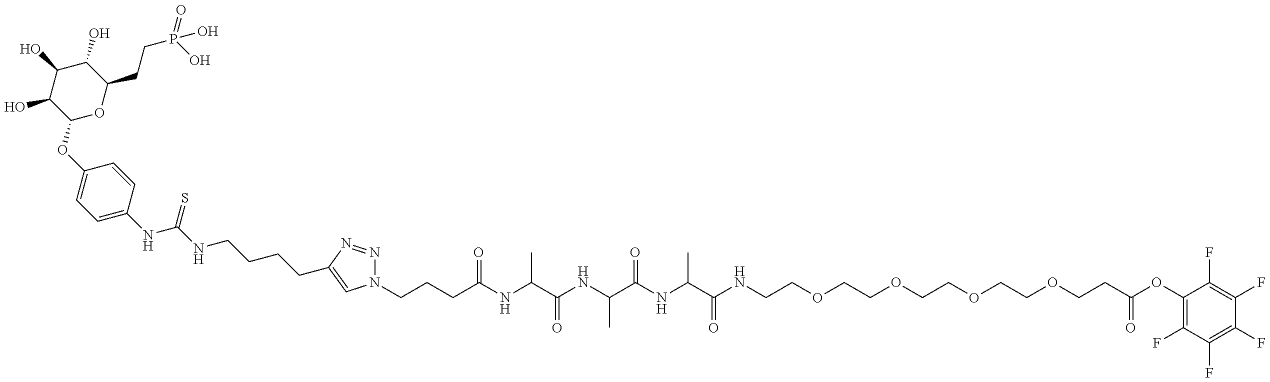 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)Oc1c(F)c(F)c(F)c(F)c1F] |
| 528 (I-51) | 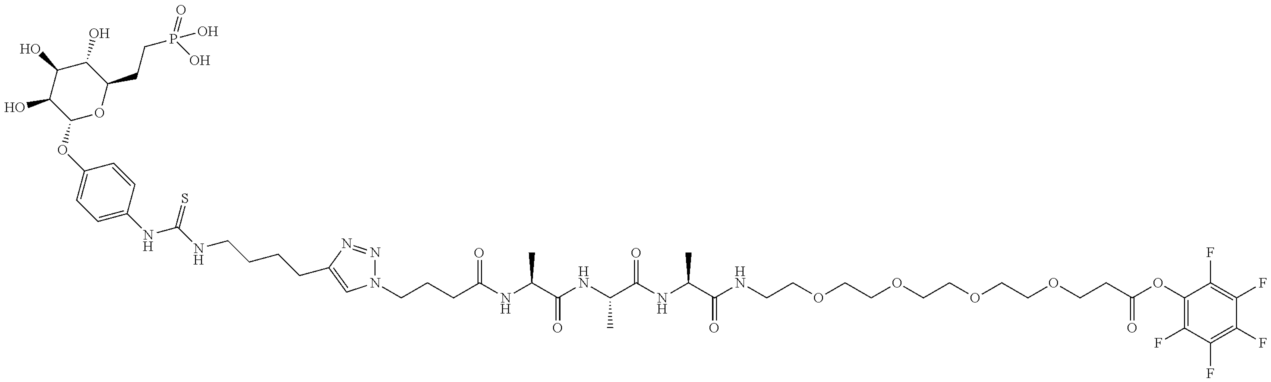 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)Oc1c(F)c(F)c(F)c(F)c1F] |
| 529 (I-38) | 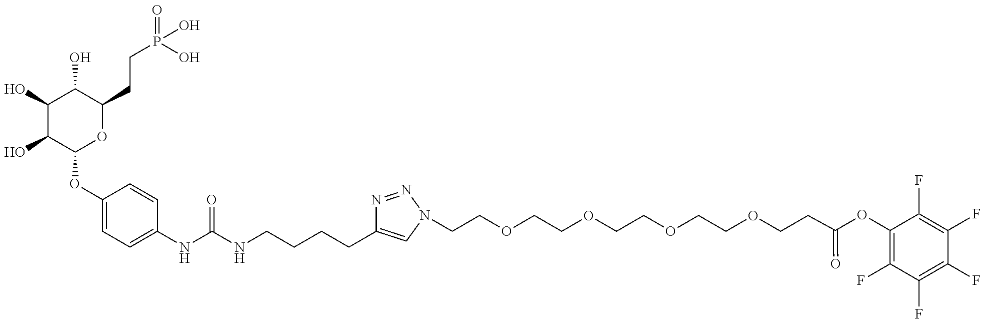 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 530 (I-50) | 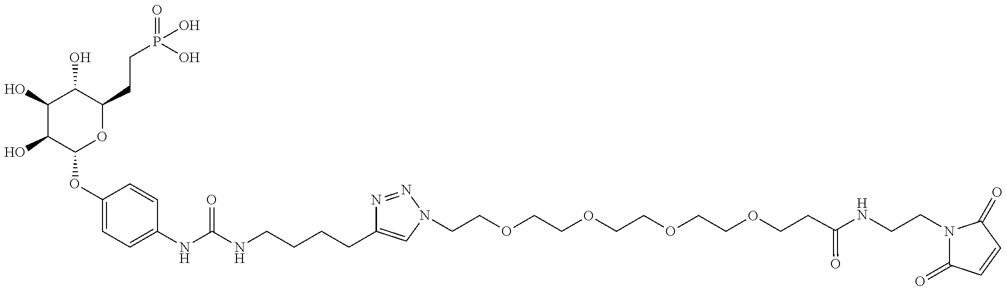 [SMILES: O=C(CCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 531 | 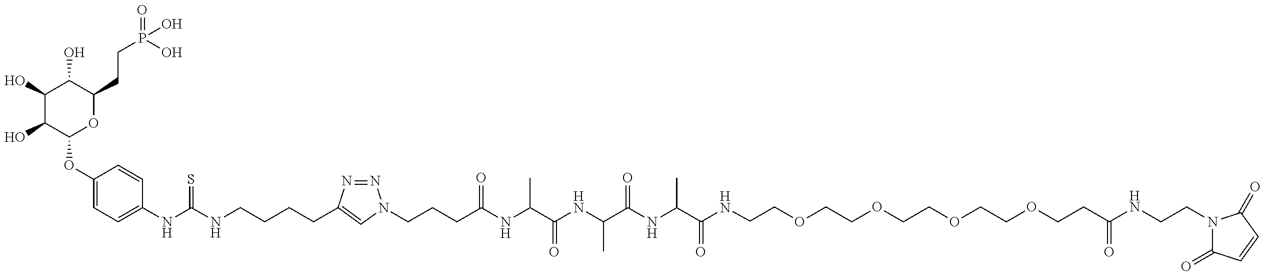 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)NCCN1C(=O)C=CC1=O] |
| 532 (I-55) | 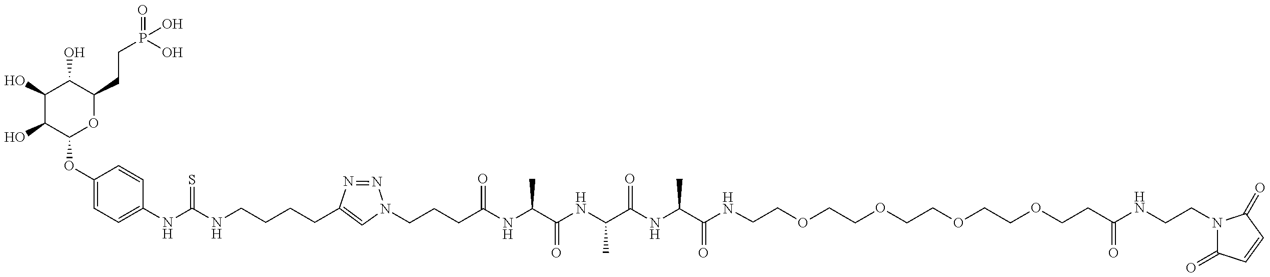 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)NCCN1C(=O)C=CC1=O] |
| 533 (I-61) | 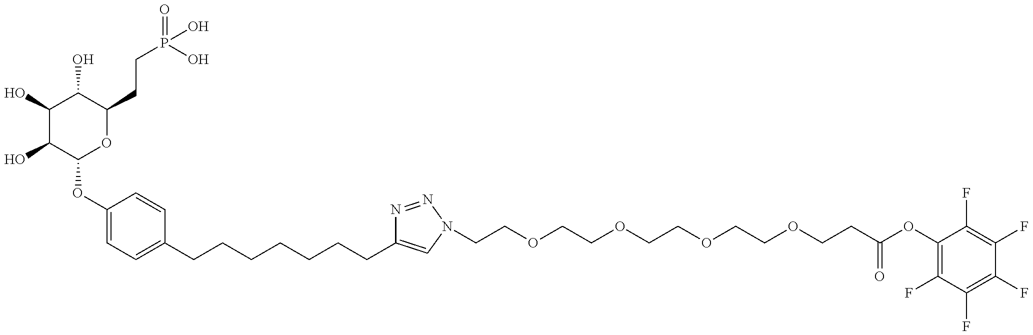 [SMILES: O=C(CCOCCOCCOCCOCCn1cc(CCCCCCCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 534 (I-62) | 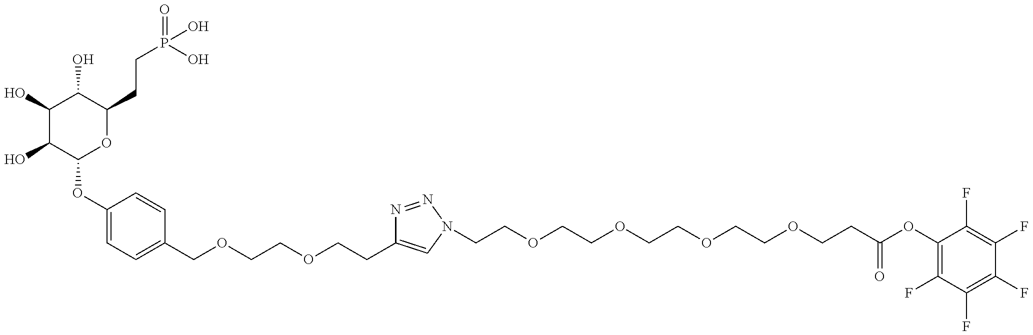 [SMILES: O=C(CCOCCOCCOCCOCCn1cc(CCOCCOCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 535 (I-88) | 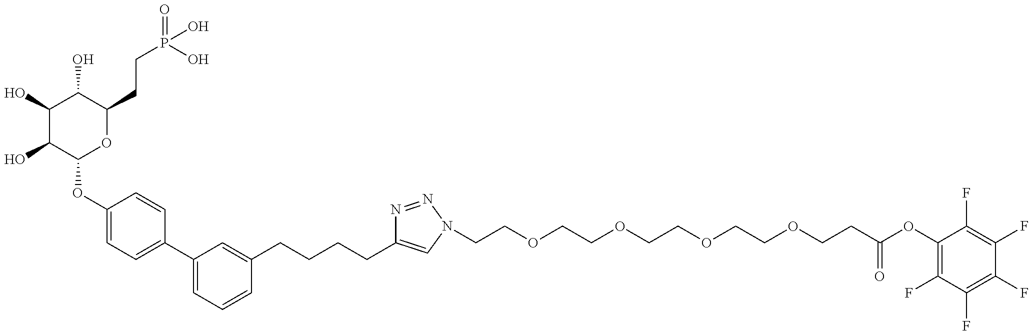 [SMILES: O=C(CCOCCOCCOCCOCCn1cc(CCCCc2cccc(-c3ccc(O[C@H]4O[C@H](CCP(=O)(O)O)[C@@H](O)[C@H](O)[C@@H]4O)cc3)c2)nn1)Oc1c(F)c(F)c(F)c(F)c1F] |
| 536 (I-60) | 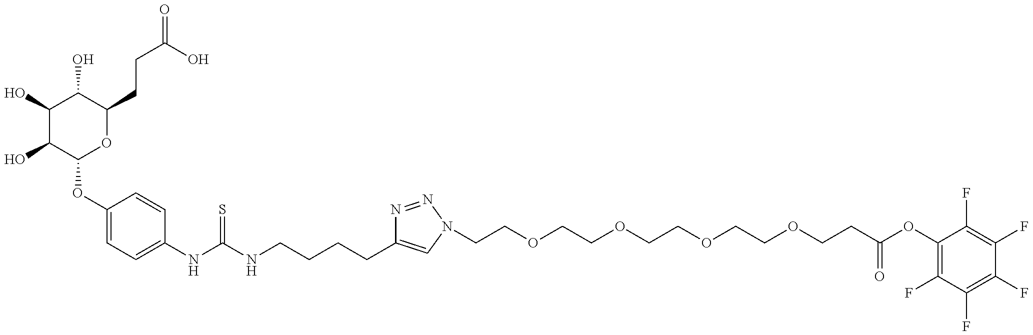 [SMILES: O=C(O)CC[C@H]1O[C@H](Oc2ccc(NC(=S)NCCCCc3cn(CCOCCOCCOCCOCCC(=O)Oc4c(F)c(F)c(F)c(F)c4F)nn3)cc2)[C@@H](O)[C@@H](O)[C@@H]1O] |
| 537 (I-66) | 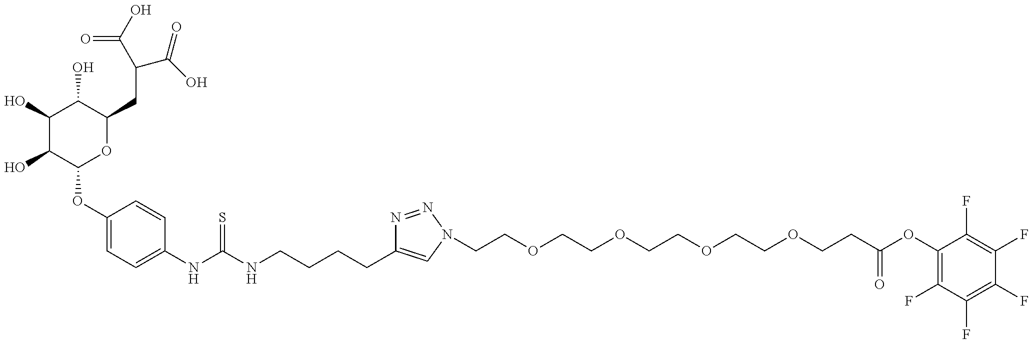 [SMILES: O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F] |
| 538 (I-64) | 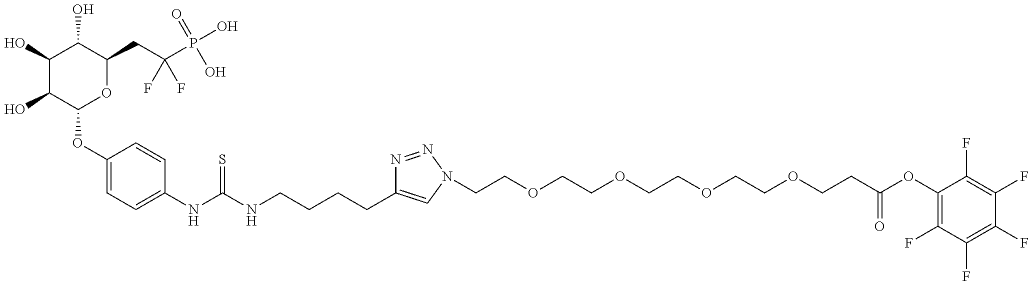 [SMILES: O=C(CCOCCOCCOCCOCCn1cc(CCCCNC(=S)Nc2ccc(O[C@H]3O[C@H](CC(F)(F)P(=O)(O)O)[C@@H](O)[C@H](O)[C@@H]3O)cc2)nn1)Oc1c(F)c(F)c(F)c(F)c1F] |
| 539 (I-65) | 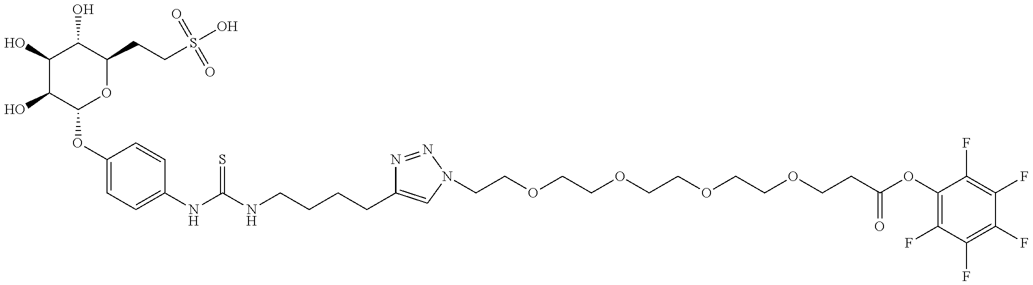 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 540 | 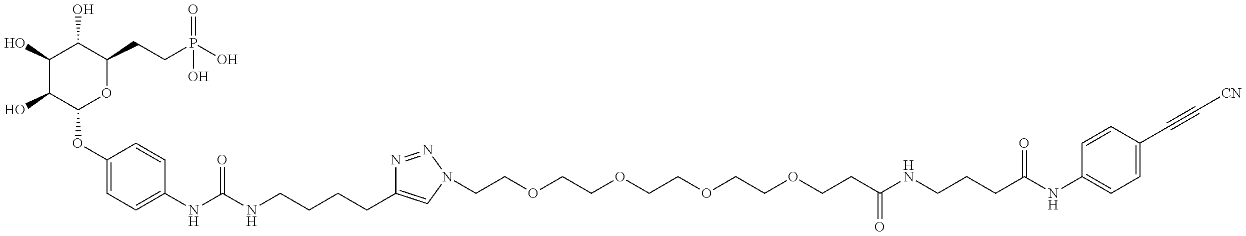 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1] |
| 541 (I-83) | 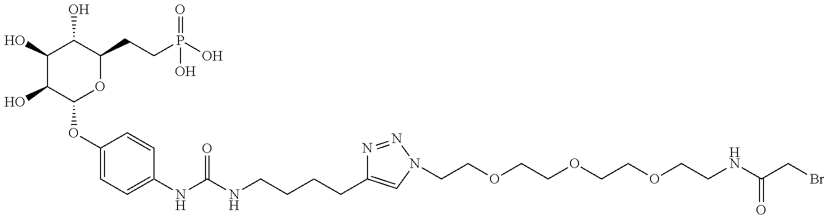 [SMILES: O=C(CBr)NCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1] |
| 542 (I-84) | 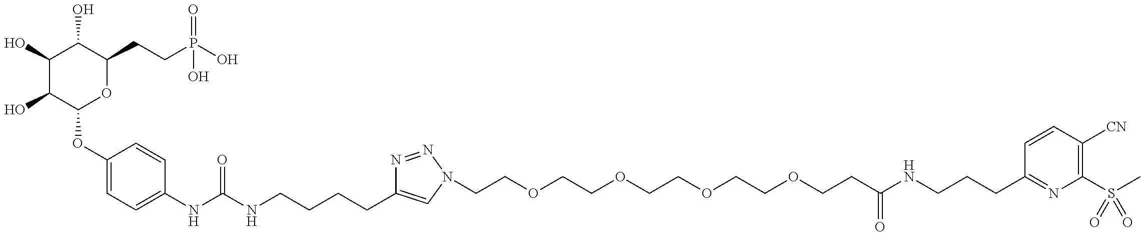 [SMILES: CS(=O)(=O)c1nc(CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)ccc1C#N] |
| 543 (I-85) | 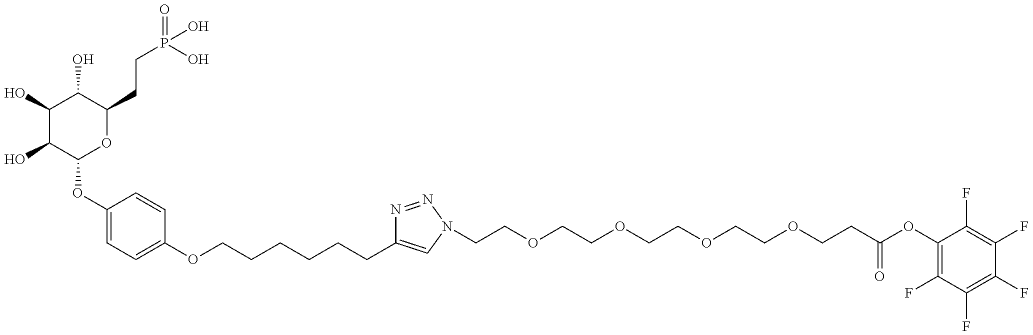 [SMILES: O=C(CCOCCOCCOCCOCCn1cc(CCCCCCOc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 544 (I-86) | 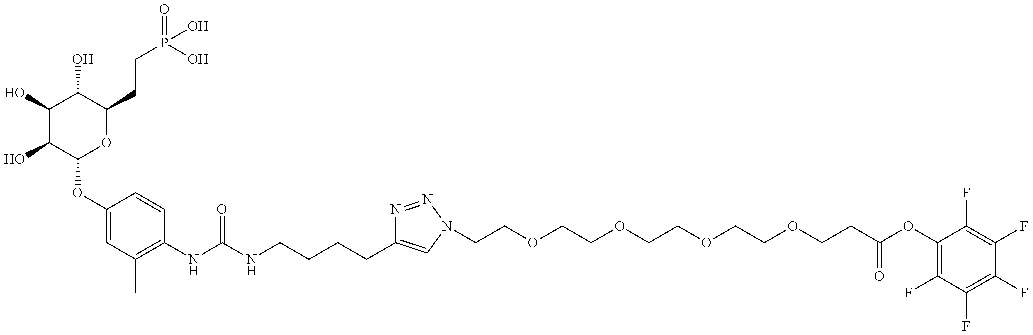 [SMILES: Cc1cc(O[C@H]2O[C@H](CCP(=O)(O)O)[C@@H](O)[C@H](O)[C@@H]2O)ccc1NC(=O)NCCCCc1cn(CCOCCOCCOCCOCCC(=O)Oc2c(F)c(F)c(F)c(F)c2F)nn1] |
| 545 (I-187) | 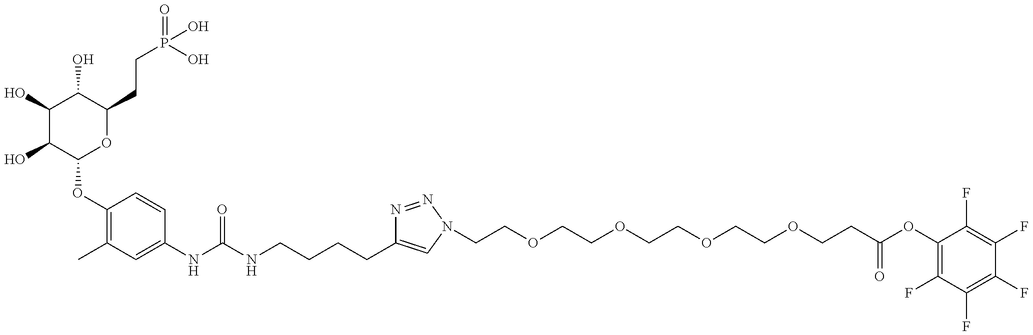 [SMILES: Cc1cc(NC(=O)NCCCCc2cn(CCOCCOCCOCCOCCC(=O)Oc3c(F)c(F)c(F)c(F)c3F)nn2)ccc1O[C@H]1O[C@H](CCP(=O)(O)O)[C@@H](O)[C@H](O)[C@@H]1O] |
| 546 (I-89) | 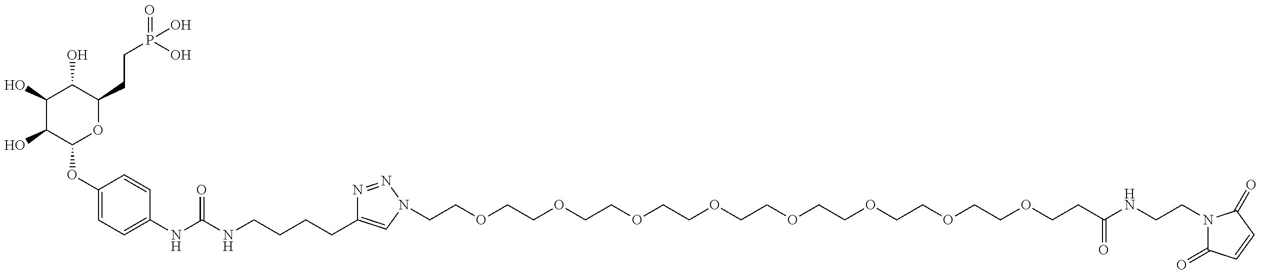 [SMILES: O=C(CCOCCOCCOCCOCCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 547 (I-90) | 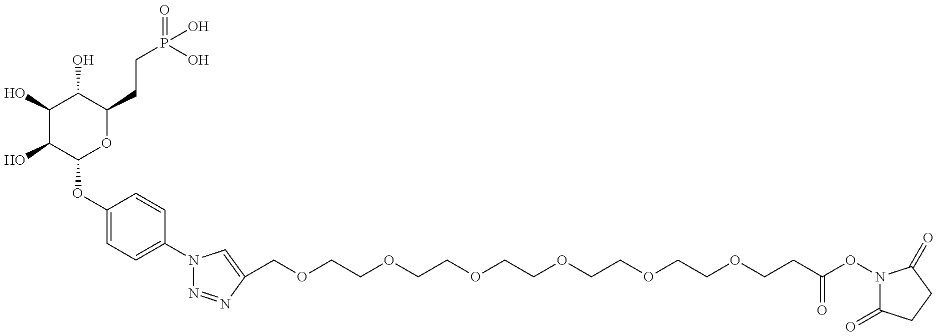 [SMILES: O=C(CCOCCOCCOCCOCCOCCOCc1cn(-c2ccc(O[C@H]3O[C@H](CCP(=O)(O)O)[C@@H](O)[C@H](O)[C@@H]3O)cc2)nn1)ON1C(=O)CCC1=O] |
| 548 (I-91) | 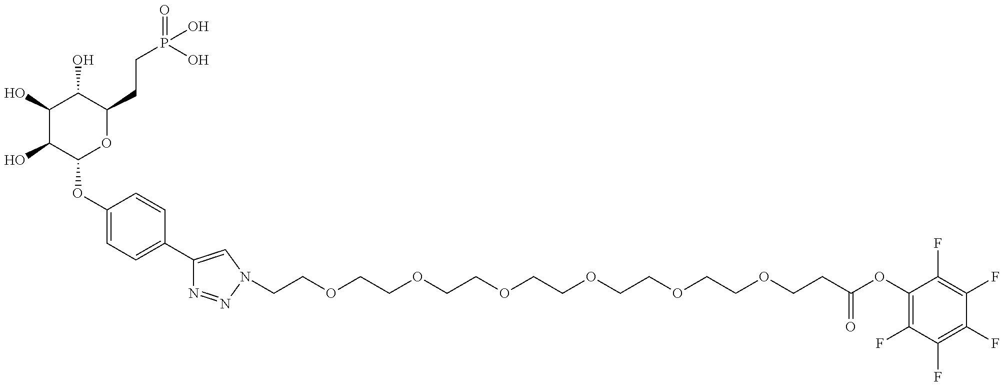 [SMILES: O=C(CCOCCOCCOCCOCCOCCOCCn1cc(-c2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 549 (I-92) | 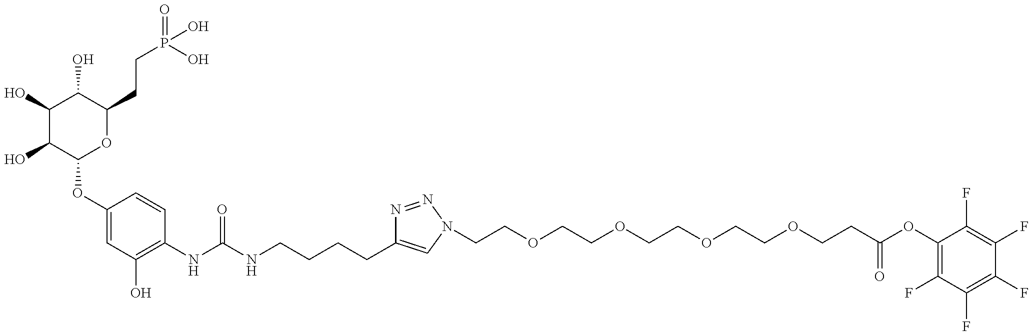 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1O] |
| 550 (I-93) | 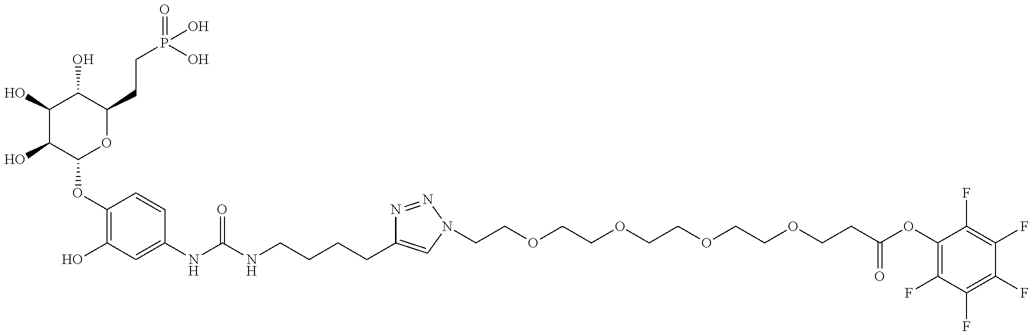 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)c(O)c1] |
| 551 (I-94) | 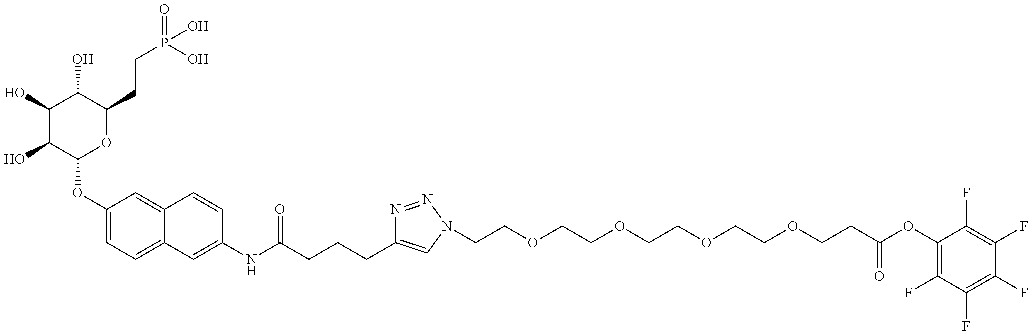 [SMILES: O=C(CCCc1cn(CCOCCOCCOCCOCCC(=O)Oc2c(F)c(F)c(F)c(F)c2F)nn1)Nc1ccc2cc(O[C@H]3O[C@H](CCP(=O)(O)O)[C@@H](O)[C@H](O)[C@@H]3O)ccc2c1] |
| 552 (I-95) | 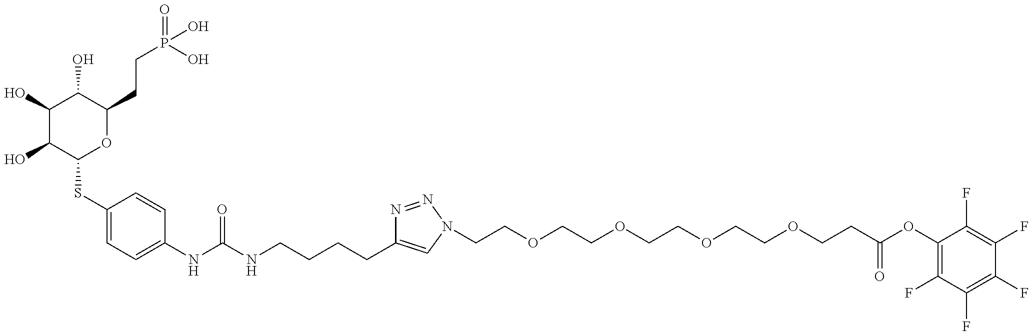 [SMILES: O=C(NCCCCc1cn(CCOCCOCCOCCOCCC(=O)Oc2c(F)c(F)c(F)c(F)c2F)nn1)Nc1ccc(S[C@H]2O[C@H](CCP(=O)(O)O)[C@@H](O)[C@H](O)[C@@H]2O)cc1] |
| 553 | 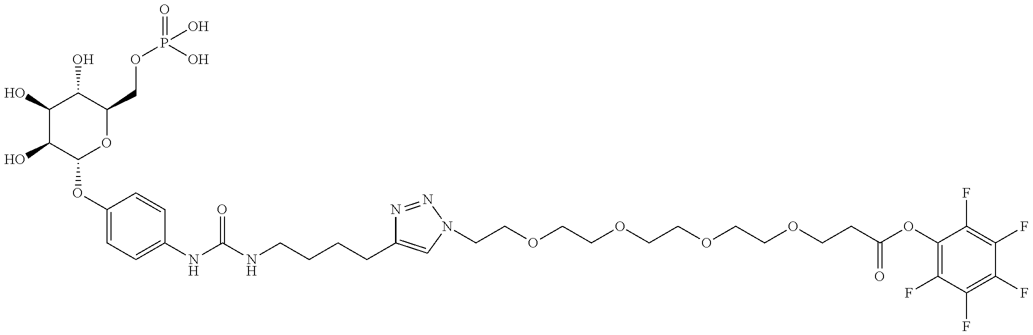 [SMILES: O=C(NCCCCc1cn(CCOCCOCCOCCOCCC(=O)Oc2c(F)c(F)c(F)c(F)c2F)nn1)Nc1ccc(O[C@H]2O[C@H](COP(=O)(O)O)[C@@H](O)[C@H](O)[C@@H]2O)cc1] |
| 554 (I- 101B) | 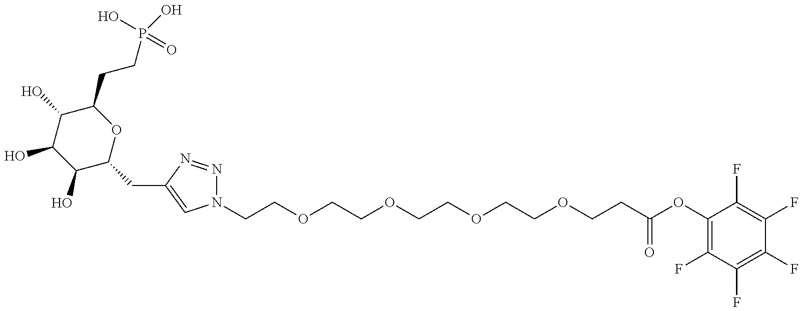 [SMILES: O=C(CCOCCOCCOCCOCCn1cc(C[C@H]2O[C@H](CCP(=O)(O)O)[C@@H](O)[C@H](O)[C@@H]2O)nn1)Oc1c(F)c(F)c(F)c(F)c1F] |
| 555 | 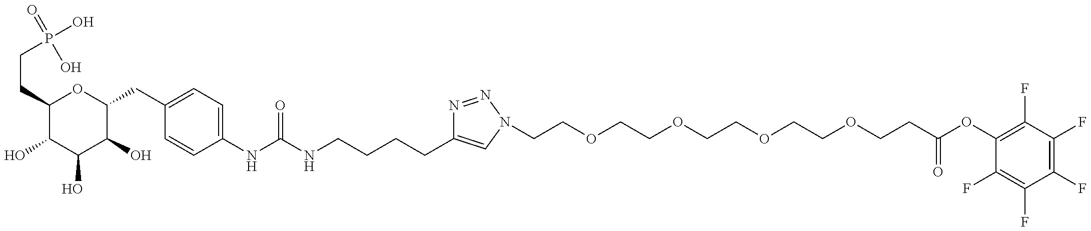 [SMILES: O=C(NCCCCc1cn(CCOCCOCCOCCOCCC(=O)Oc2c(F)c(F)c(F)c(F)c2F)nn1)Nc1ccc(C[C@H]2O[C@H](CCP(=O)(O)O)[C@@H](O)[C@H](O)[C@@H]2O)cc1] |
| 556 (I-104) | 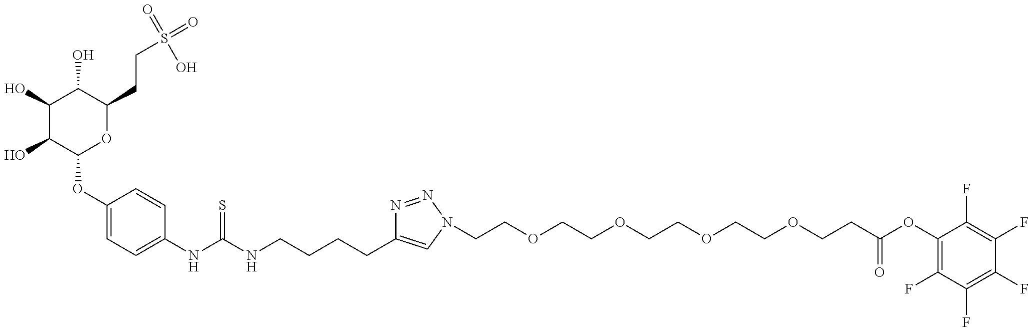 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 557 (I-103) | 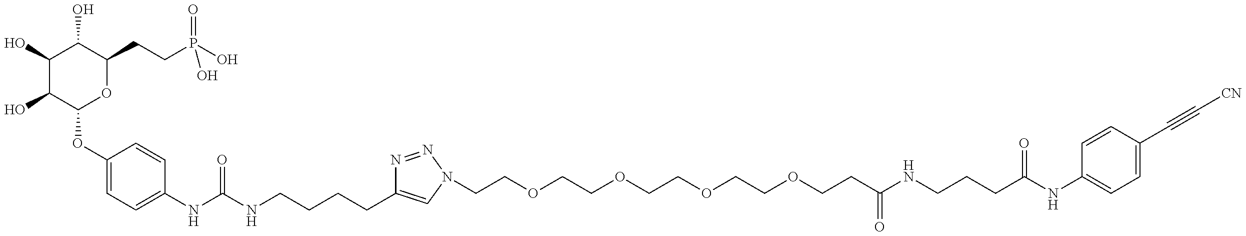 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1] |

TABLE 8
Exemplary M6PR binding compounds of formula (Ia)

| # | Compound Structure |
|---|---|
| 601 (I-8) | 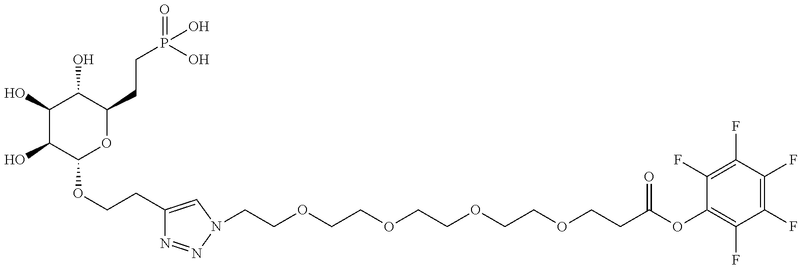 [SMILES: O=C(CCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 602 (I-9) | 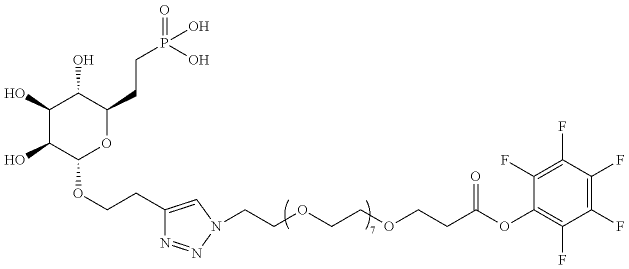 |
| 603 (I-10) | 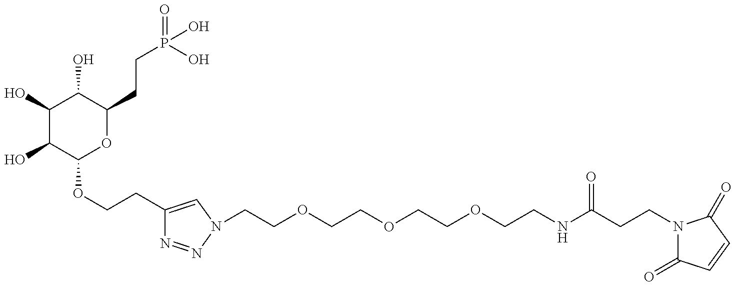 [SMILES: O=C(CCN1C(=O)C=CC1=O)NCCOCCOCCOCCn1cc(CCO[C@H]2O[C@H](CCP(=O)(O)O)[C@@H](O)[C@H](O)[C@@H]2O)nn1] |
| 604 (I-11) | 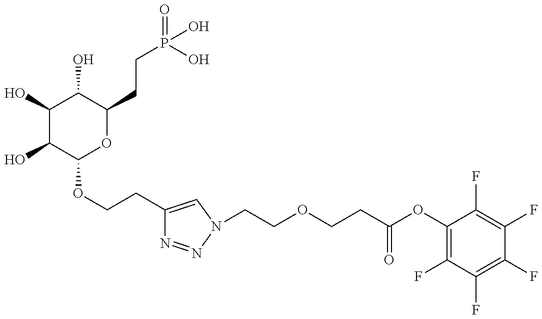 [SMILES: O=C(CCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 605 | 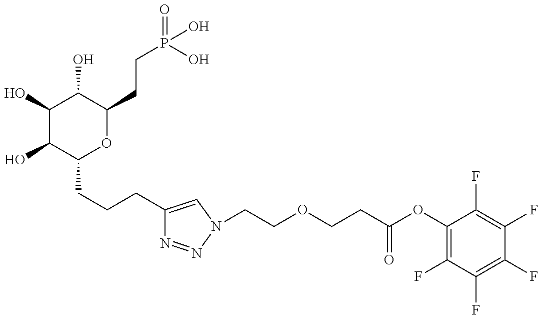 [SMILES: O=C(CCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 606 (I-13) | 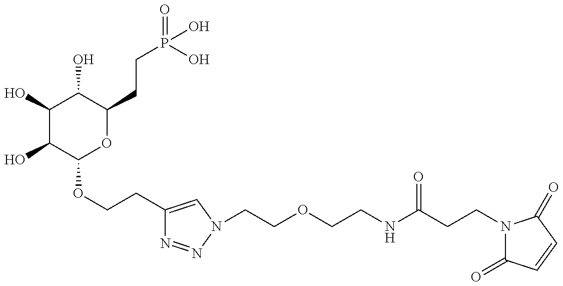 [SMILES: O=C(CCN1C(=O)C=CC1=O)NCCOCCn1cc(CCO[C@H]2O[C@H](CCP(=O)(O)O)[C@@H](O)[C@H](O)[C@@H]2O)nn1] |
| 607 (I-14) | 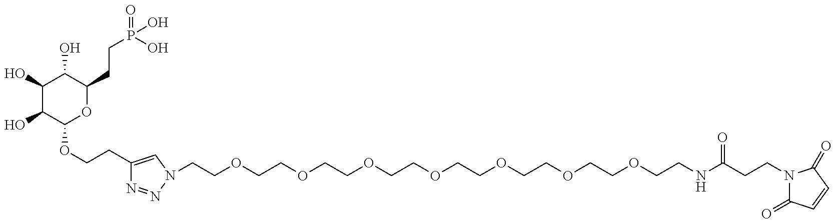 [SMILES: O=C(CCN1C(=O)C=CC1=O)NCCOCCOCCOCCOCCOCCOCCOCCn1cc(CCO[C@H]2O[C@H](CCP(=O)(O)O)[C@@H](O)[C@H](O)[C@@H]2O)nn1] |
| 608 (I-15) | 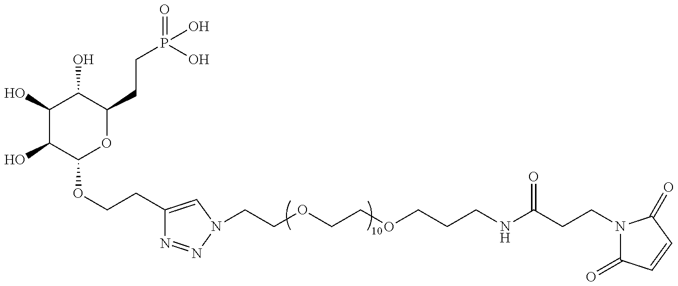 |
| 609 | 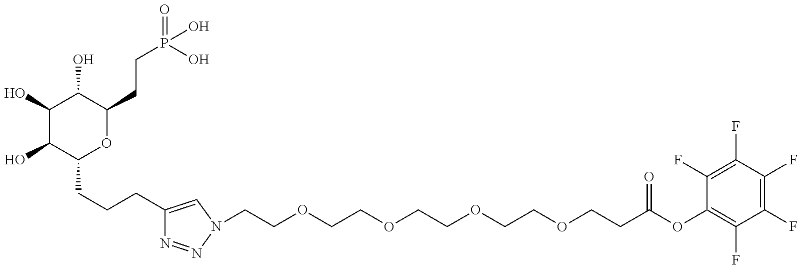 [SMILES: O=C(CCOCCOCCOCCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 610 | 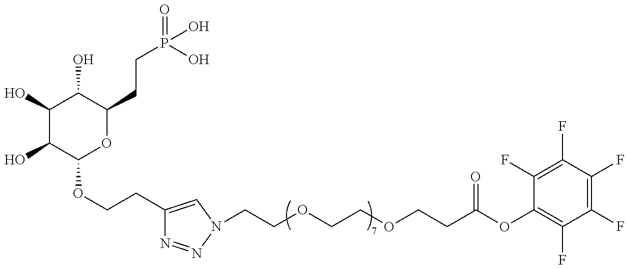 |
| 611 | 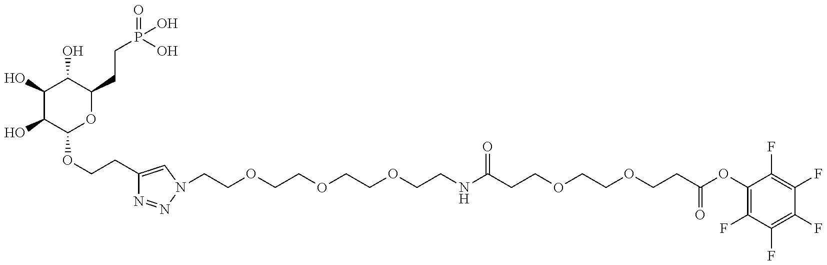 [SMILES: O=C(CCOCCOCCC(=O)Oc1c(F)c(F)c(F)c(F)c1F)NCCOCCOCCOCCn1cc(CCO[C@H]2O[C@H](CCP(=O)(O)O)[C@@H](O)[C@H](O)[C@@H]2O)nn1] |
| 612 (k = 4, l = 0) (I-33) 613 (k = 0, l = 12) (I-34) 614 (k = 2, l = 6), (I-35) | 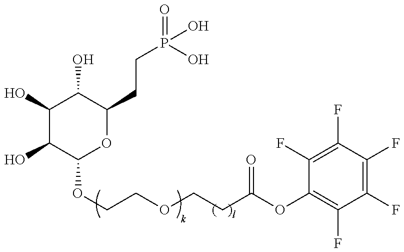 |
| 615 | 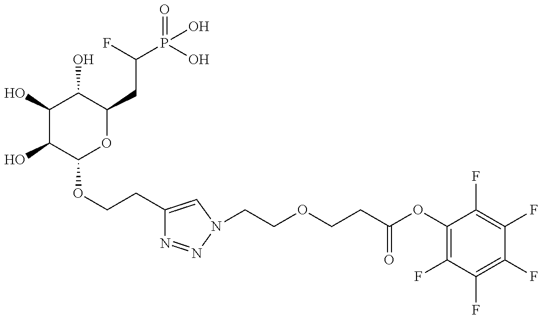 [SMILES: O=C(CCOCCn1cc(CCO[C@H]2O[C@H](CC(F)P(=O)(O)O)[C@@H](O)[C@H](O)[C@@H]2O)nn1)Oc1c(F)c(F)c(F)c(F)c1F] |
| 616 | 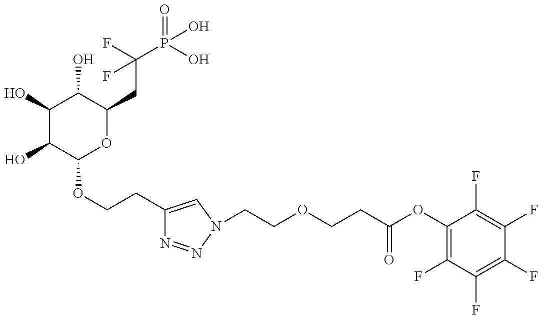 [SMILES: O=C(CCOCCn1cc(CCO[C@H]2O[C@H](CC(F)(F)P(=O)(O)O)[C@@H](O)[C@H](O)[C@@H]2O)nn1)Oc1c(F)c(F)c(F)c(F)c1F] |
| 617 | 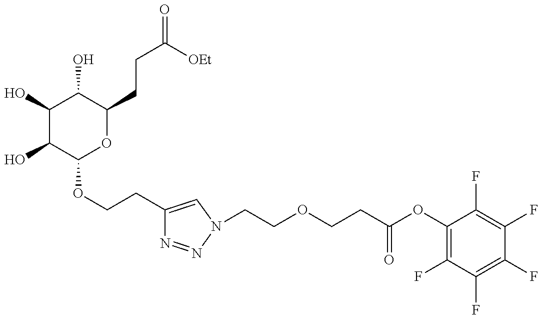 [SMILES: CCOC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 618 | 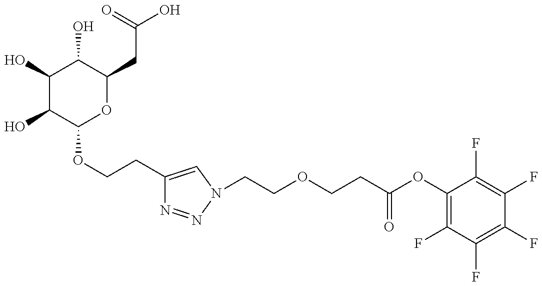 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 619 | 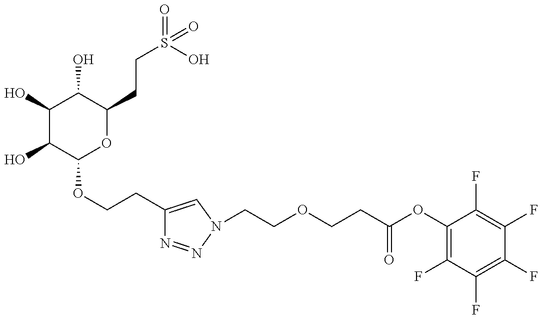 [SMILES: O=C(CCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 620 | 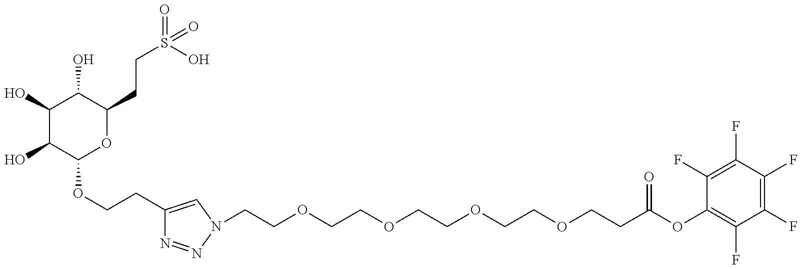 [SMILES: O=C(CCOCCOCCOCCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 621 | 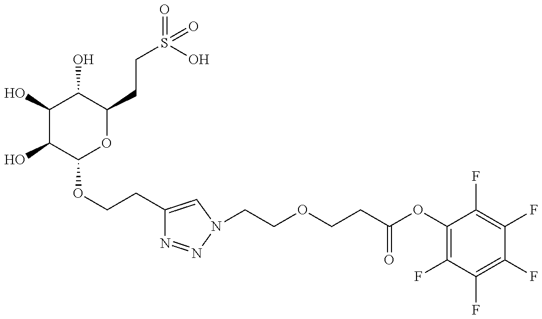 [SMILES: O=C(CCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 622 | 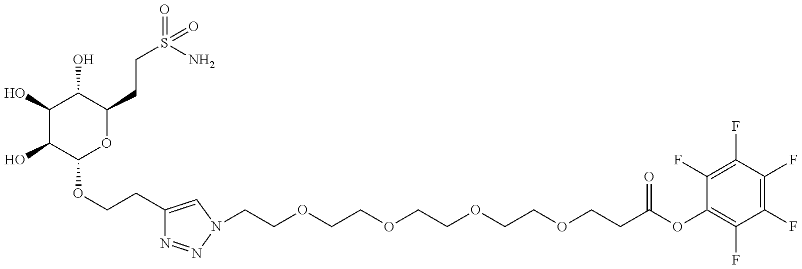 [SMILES: NS(=O)(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 623 | 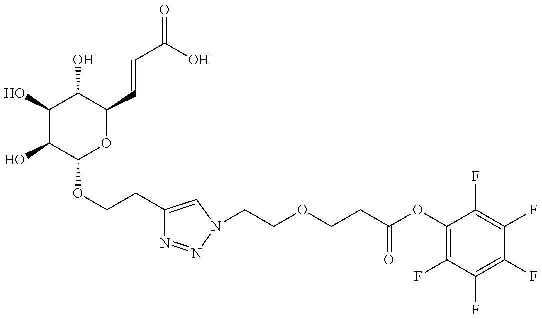 [SMILES: O=C(O)/C=C/[C@H]1O[C@H](OCCc2cn(CCOCCC(=O)Oc3c(F)c(F)c(F)c(F)c3F)nn2)[C@@H](O)[C@@H](O)[C@@H]1O] |
| 624 | 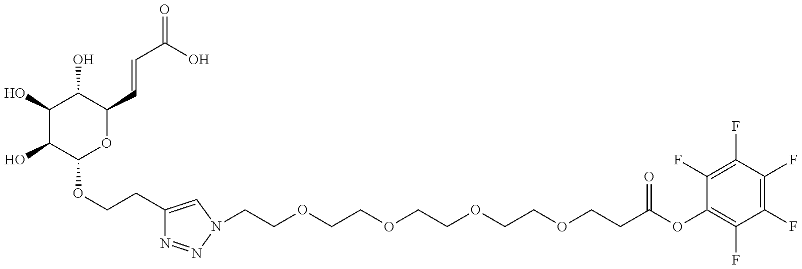 [SMILES: O=C(O)/C=C/[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 625 | 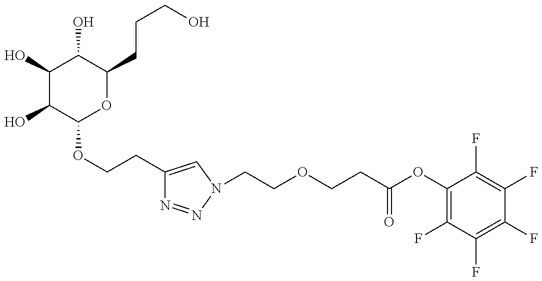 [SMILES: O=C(CCOCCn1cc(CCO[C@H]2O[C@H](CCCO)[C@@H](O)[C@H](O)[C@@H]2O)nn1)Oc1c(F)c(F)c(F)c(F)c1F] |
| 626 | 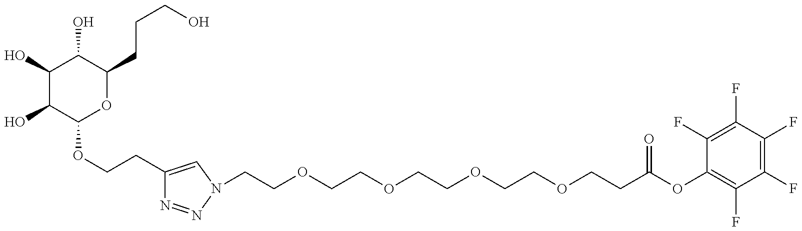 [SMILES: O=C(CCOCCOCCOCCOCCn1cc(CCO[C@H]2O[C@H](CCCO)[C@@H](O)[C@H](O)[C@@H]2O)nn1)Oc1c(F)c(F)c(F)c(F)c1F] |
| 627 | 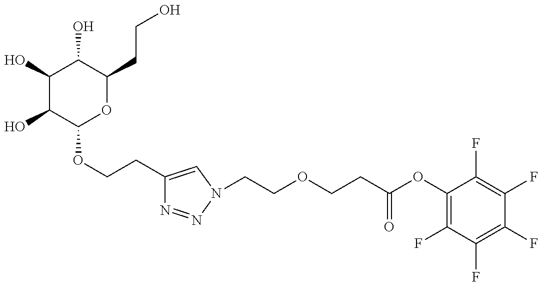 [SMILES: O=C(CCOCCn1cc(CCO[C@H]2O[C@H](CCO)[C@@H](O)[C@H](O)[C@@H]2O)nn1)Oc1c(F)c(F)c(F)c(F)c1F] |
| 628 | 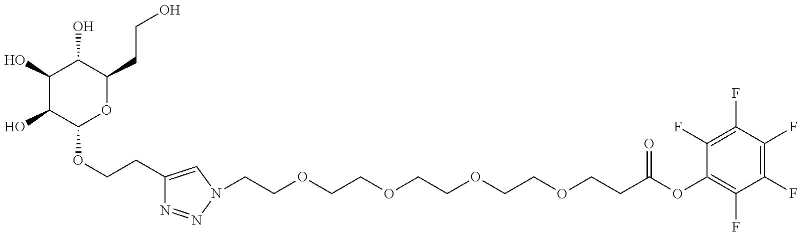 [SMILES: O=C(CCOCCOCCOCCOCCn1cc(CCO[C@H]2O[C@H](CCO)[C@@H](O)[C@H](O)[C@@H]2O)nn1)Oc1c(F)c(F)c(F)c(F)c1F] |
| 629 | 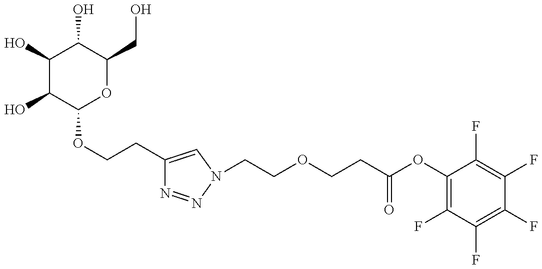 [SMILES: O=C(CCOCCn1cc(CCO[C@H]2O[C@H](CO)[C@@H](O)[C@H](O)[C@@H]2O)nn1)Oc1c(F)c(F)c(F)c(F)c1F] |
| 630 | 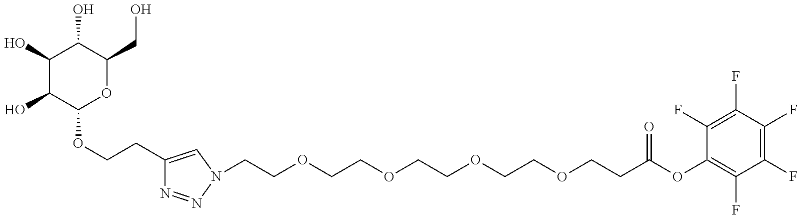 [SMILES: O=C(CCOCCOCCOCCOCCn1cc(CCO[C@H]2O[C@H](CO)[C@@H](O)[C@H](O)[C@@H]2O)nn1)Oc1c(F)c(F)c(F)c(F)c1F] |
| 631 | 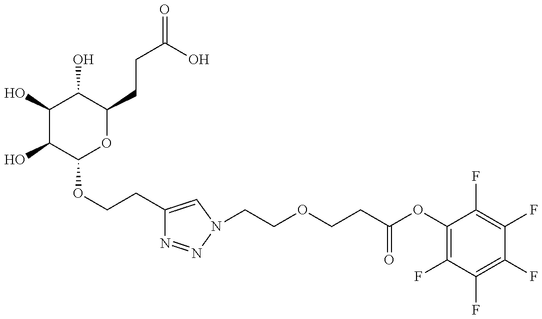 [SMILES: O=C(O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 632 | 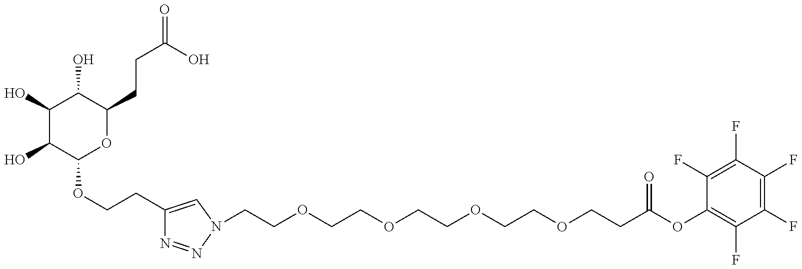 [SMILES: O=C(O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 633 | 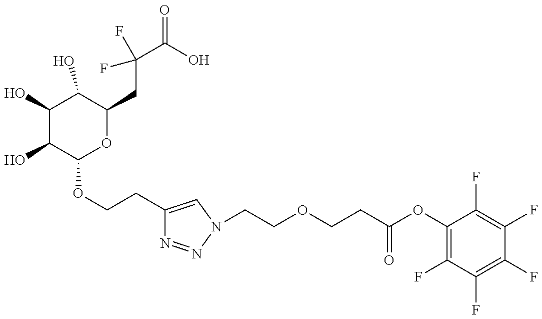 [SMILES: O=C(CCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 634 | 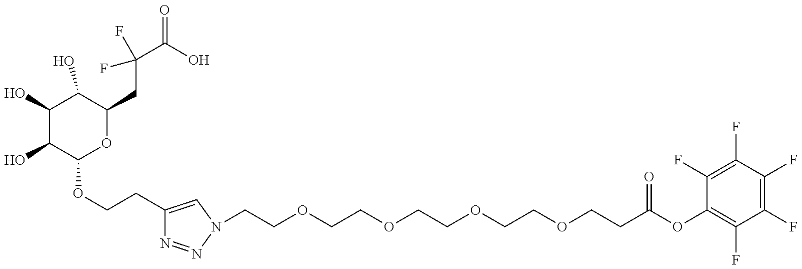 [SMILES: O=C(CCOCCOCCOCCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 635 | 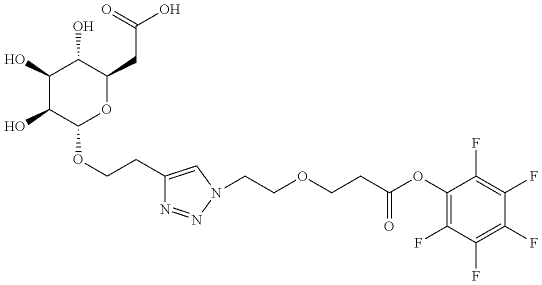 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 636 | 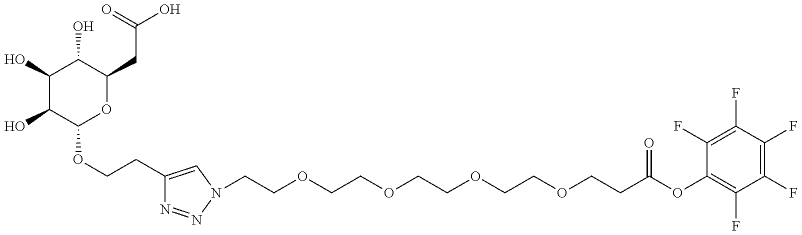 [SMILES: O=C(O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 637 | 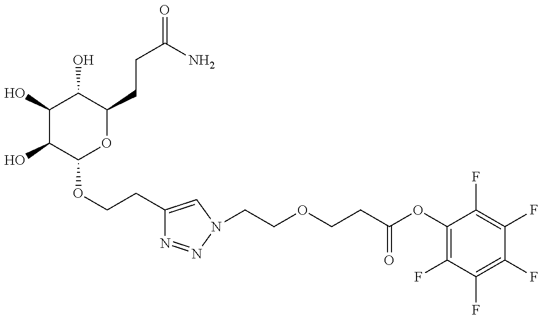 [SMILES: NC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 638 | 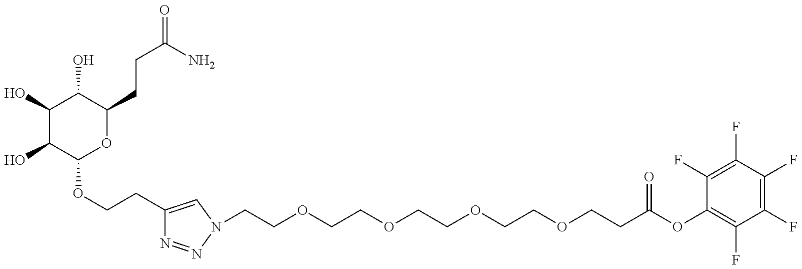 [SMILES: NC(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 639 | 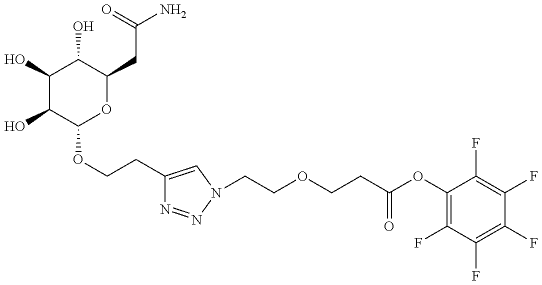 [SMILES: NC(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 640 | 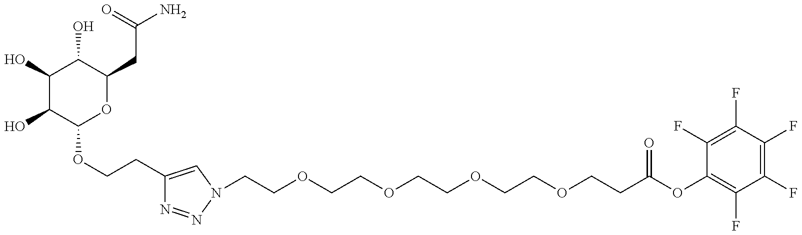 [SMILES: NC(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 641 | 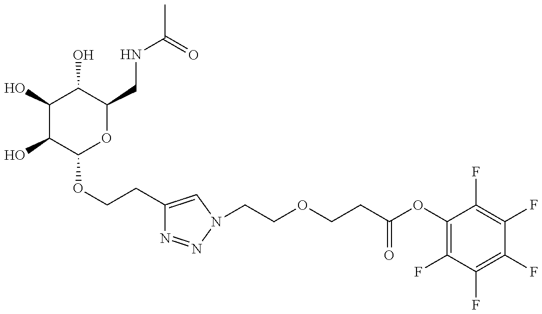 [SMILES: CC(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 642 | 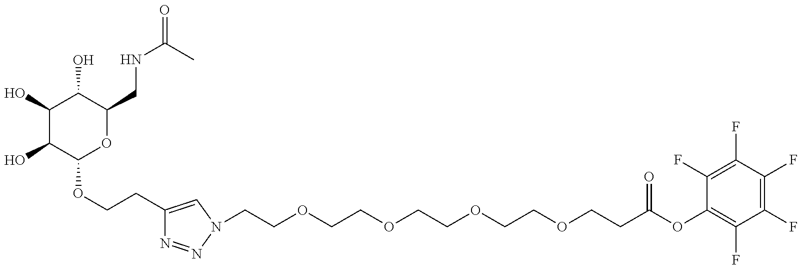 [SMILES: CC(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 643 | 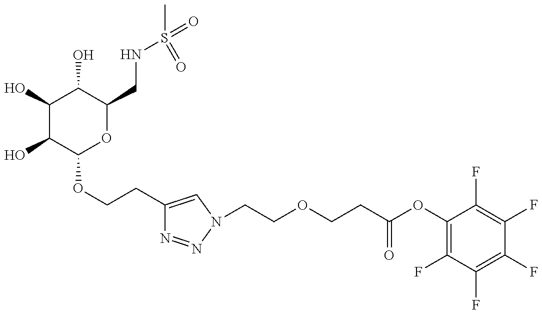 [SMILES: CS(=O)(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 644 | 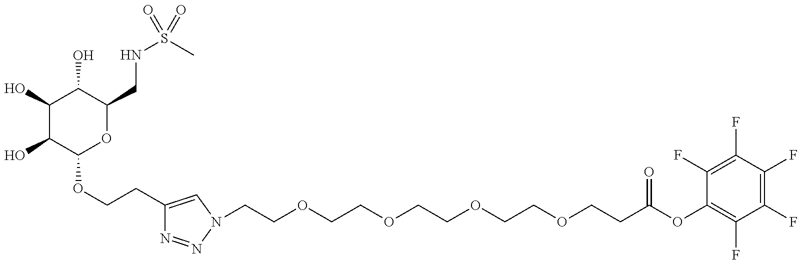 [SMILES: CS(=O)(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 645 | 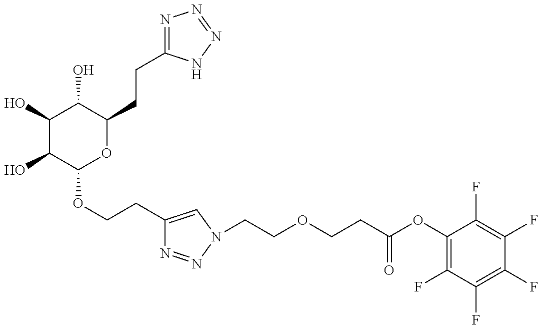 [SMILES: O=C(CCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 646 | 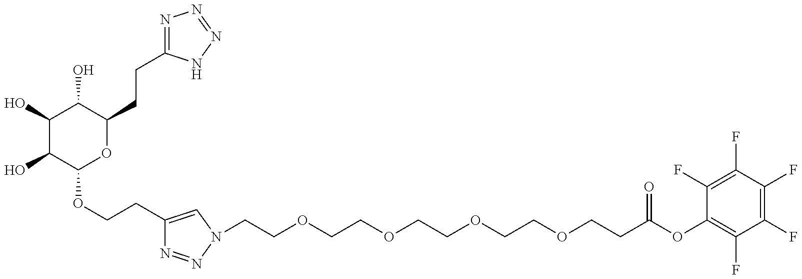 [SMILES: O=C(CCOCCOCCOCCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 647 | 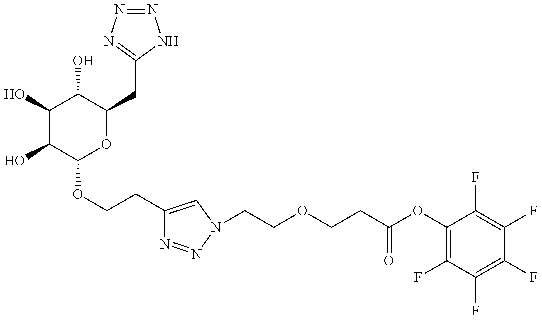 [SMILES: O=C(CCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 648 | 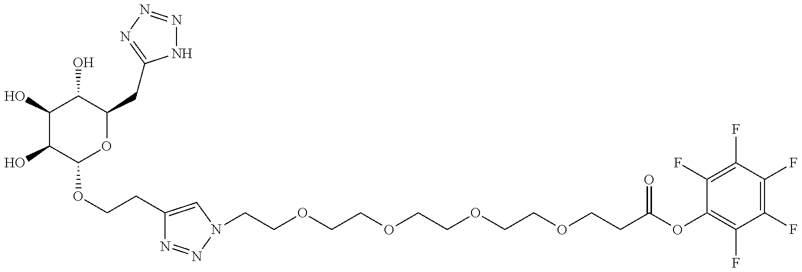 [SMILES: O=C(CCOCCOCCOCCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 649 | 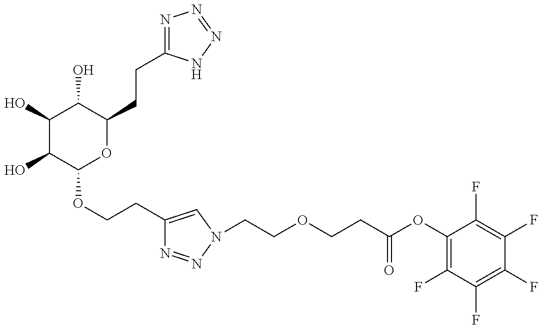 [SMILES: O=C(CCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 650 | 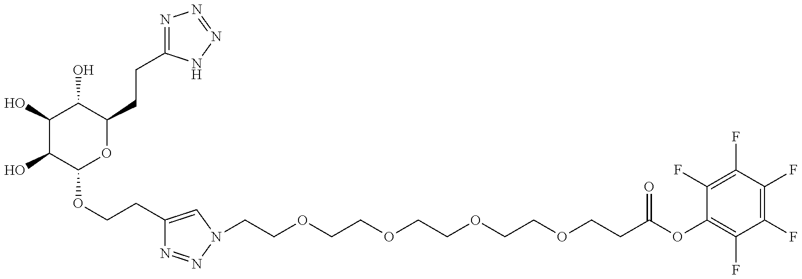 [SMILES: O=C(CCOCCOCCOCCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 651 | 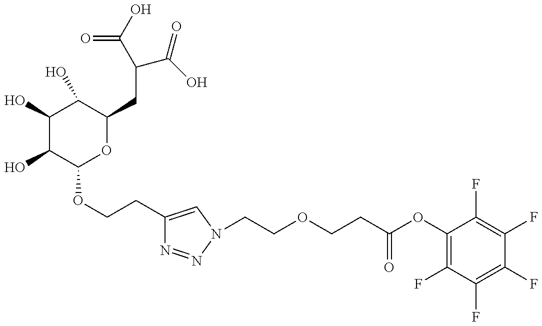 [SMILES: O=C(CCOCCn1cc(CCO[C@H]2O[C@H](CC(C(=O)O)C(=O)O)[C@@H](O)[C@H](O)[C@@H]2O)nn1)Oc1c(F)c(F)c(F)c(F)c1F] |
| 652 | 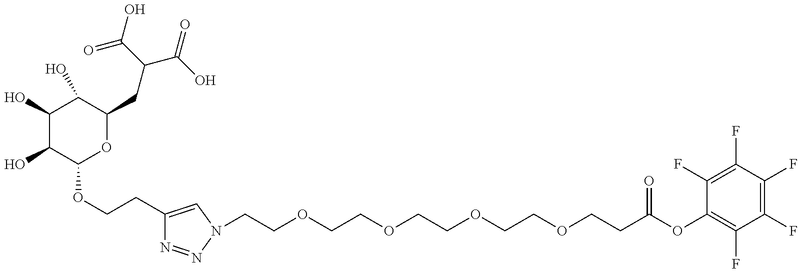 [SMILES: O=C(CCOCCOCCOCCOCCn1cc(CCO[C@H]2O[C@H](CC(C(=O)O)C(=O)O)[C@@H](O)[C@H](O)[C@@H]2O)nn1)Oc1c(F)c(F)c(F)c(F)c1F] |
| 653 | 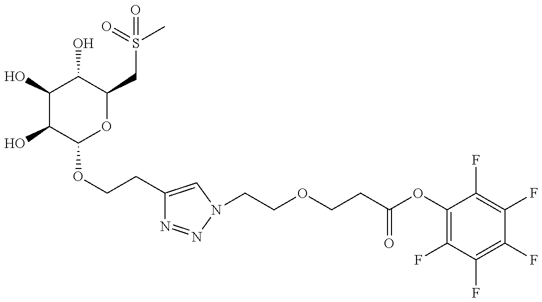 [SMILES: CS(=O)(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 654 | 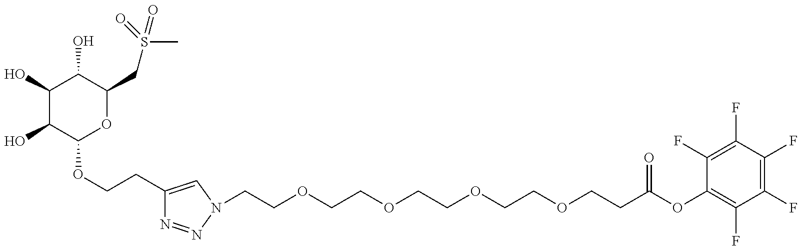 [SMILES: CS(=O)(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 656 (I-37) | 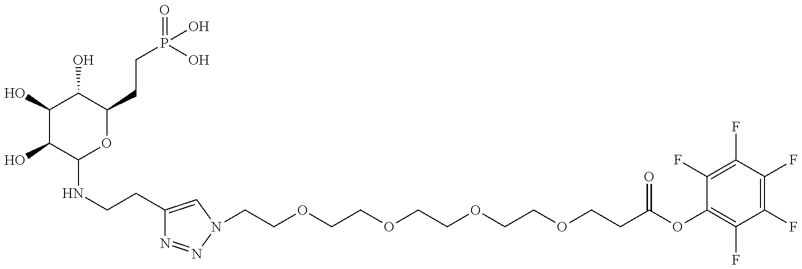 [SMILES: O=C(CCOCCOCCOCCOCCn1cc(CCNC2O[C@H](CCP(=O)(O)O)[C@@H](O)[C@H](O)[C@@H]2O)nn1)Oc1c(F)c(F)c(F)c(F)c1F] |

In certain embodiments of formula (Ia), n is 2. In certain embodiments of formula (Ia)-(Ib), n is 2, and Y is a chemoselective ligation group that can be conjugated to a bridging moiety. In certain embodiments of formula (Ia), n is 3. In certain embodiments of formula (Ta), n is 3, and Y is a chemoselective ligation group.

Exemplary multivalent M6PR binding compounds for use in preparing a bridging composition of this disclosure are shown in Tables 9-10.

Exemplary multivalent ASGPR binding compounds for use in preparing a bridging composition of this disclosure are shown in Tables 11-12.

TABLE 9
Multivalent M6PR binding compounds having chemo selective ligation group

| # | Structure |
|---|---|
| 701 (I-12) | 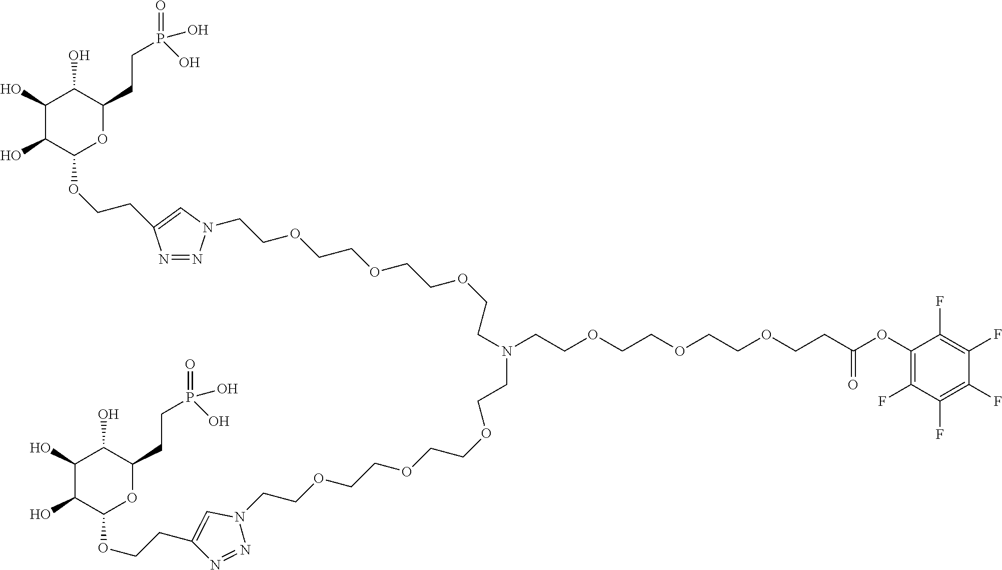 [SMILES: O=C(CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 702 | 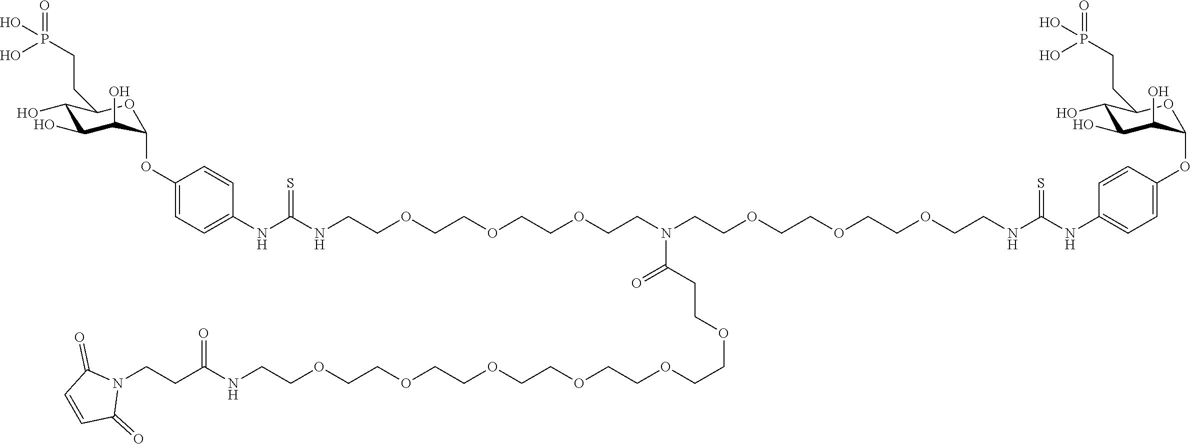 |
| 703 | 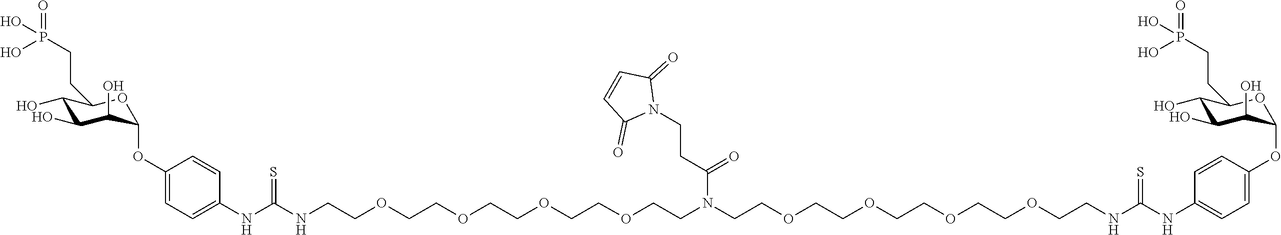 |
| 704 | 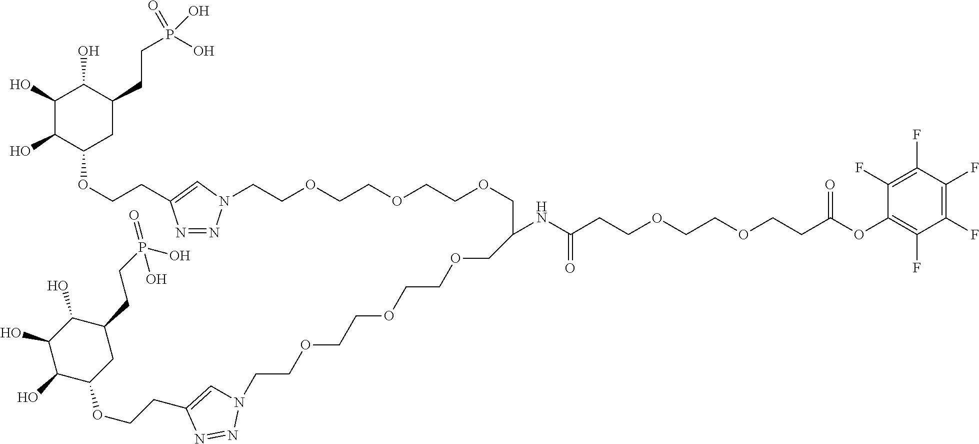 [SMILES: O=C(CCOCCOCCC(=O)Oc1c(F)c(F)c(F)c(F)c1F)NC(COCCOCCOCCn1cc(CCO[C@H]2C[C@H](CCP(=O)(O)O)[C@@H](O)[C@H](O)[C@@H]2O)nn1)COCCOCCOCCn1cc(CCO[C@H]2C[C@H](CCP(=O)(O)O)[C@@H](O)[C@H](O)[C@@H]2O)nn1] |
| 705 (I-40) | 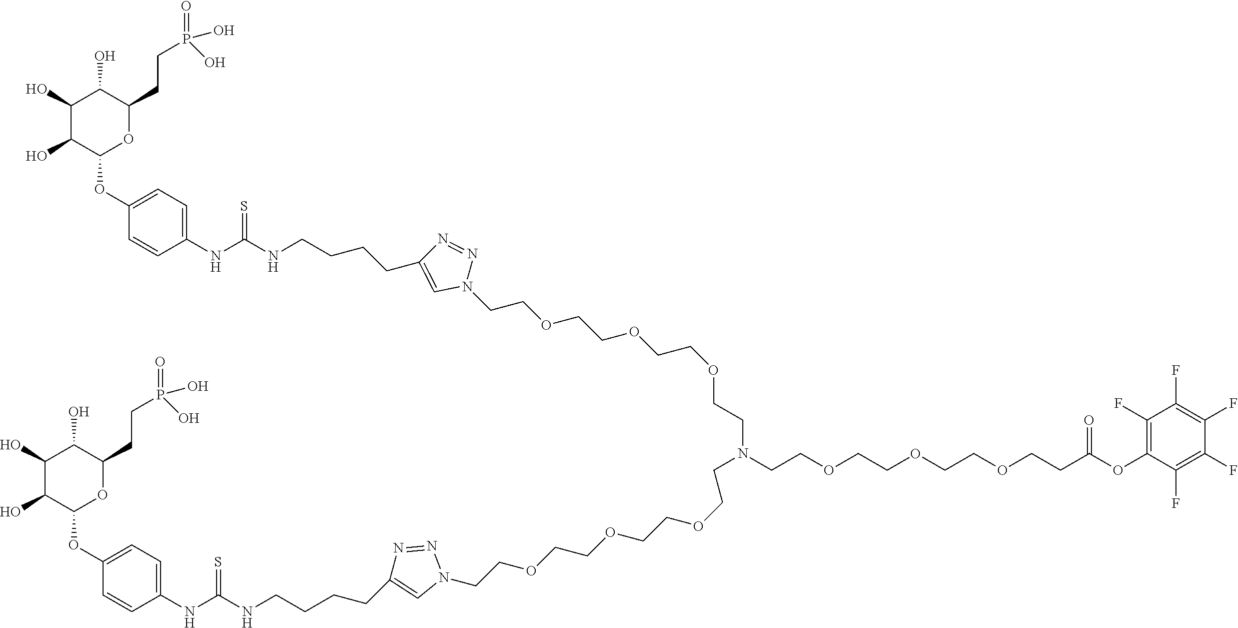 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 706 (I-41) | 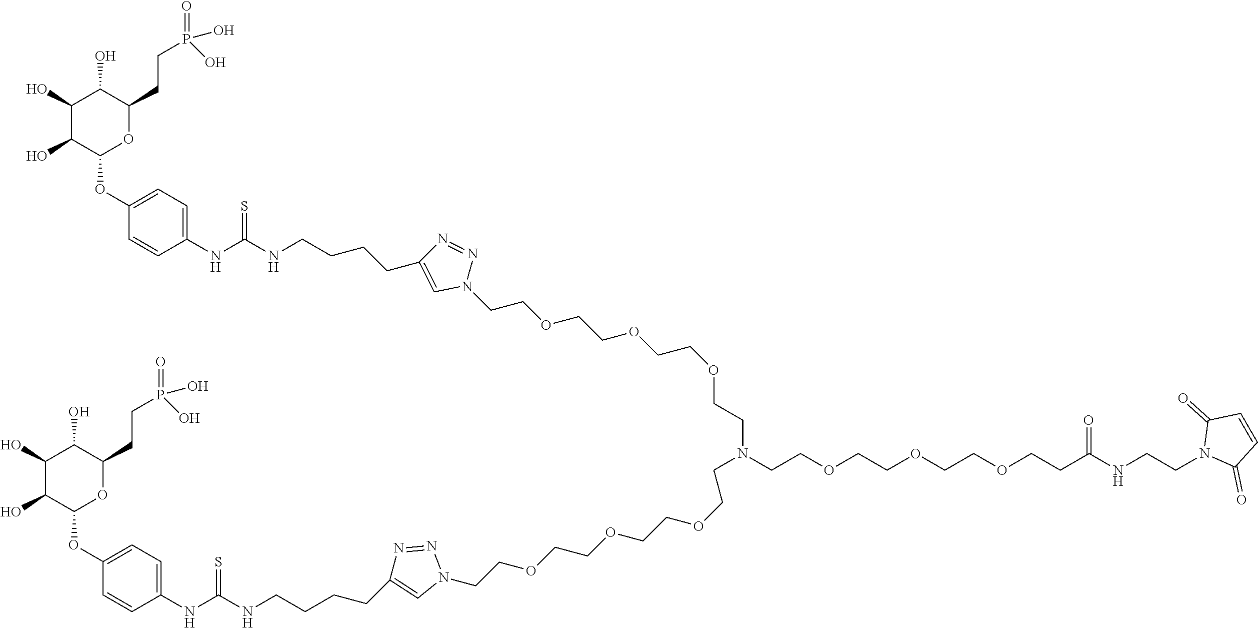 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 707 (I-43) | 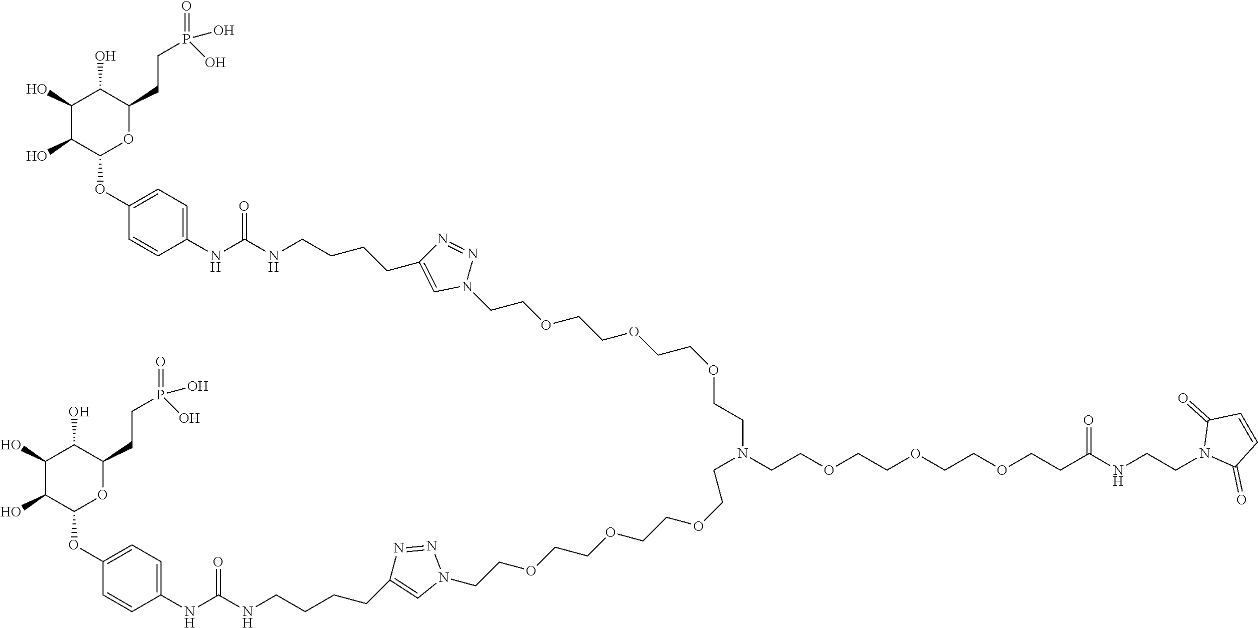 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 708 (I-58) | 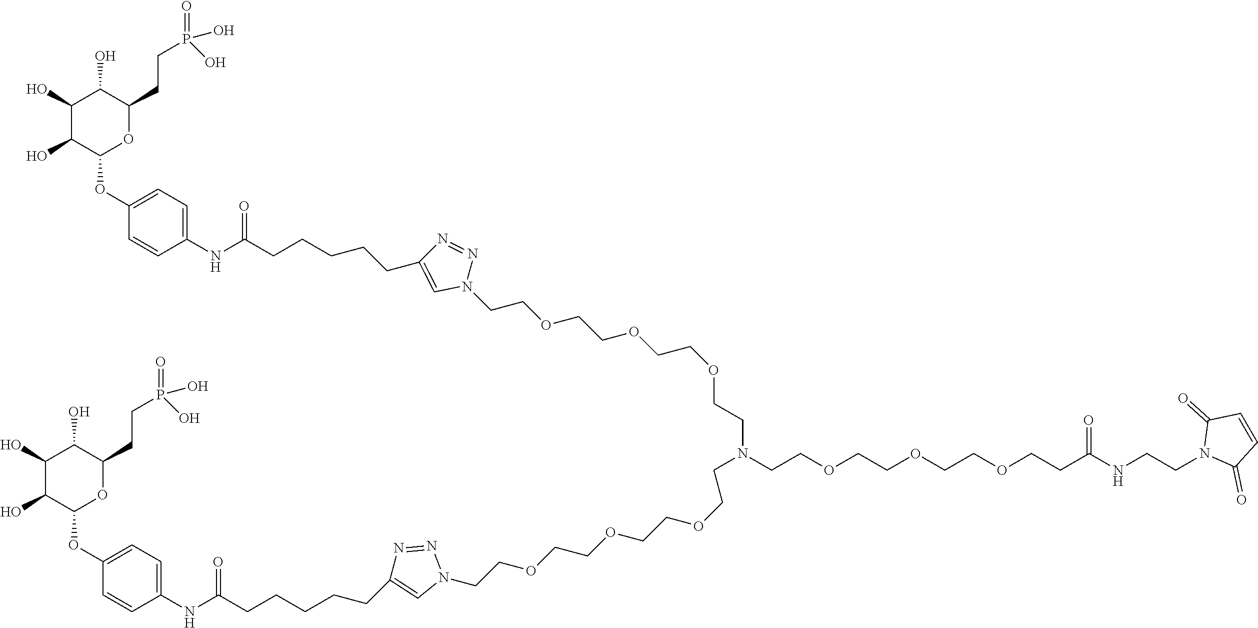 [SMILES: O=C(CCOCCOCCOCCN(CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 709 (I-42) | 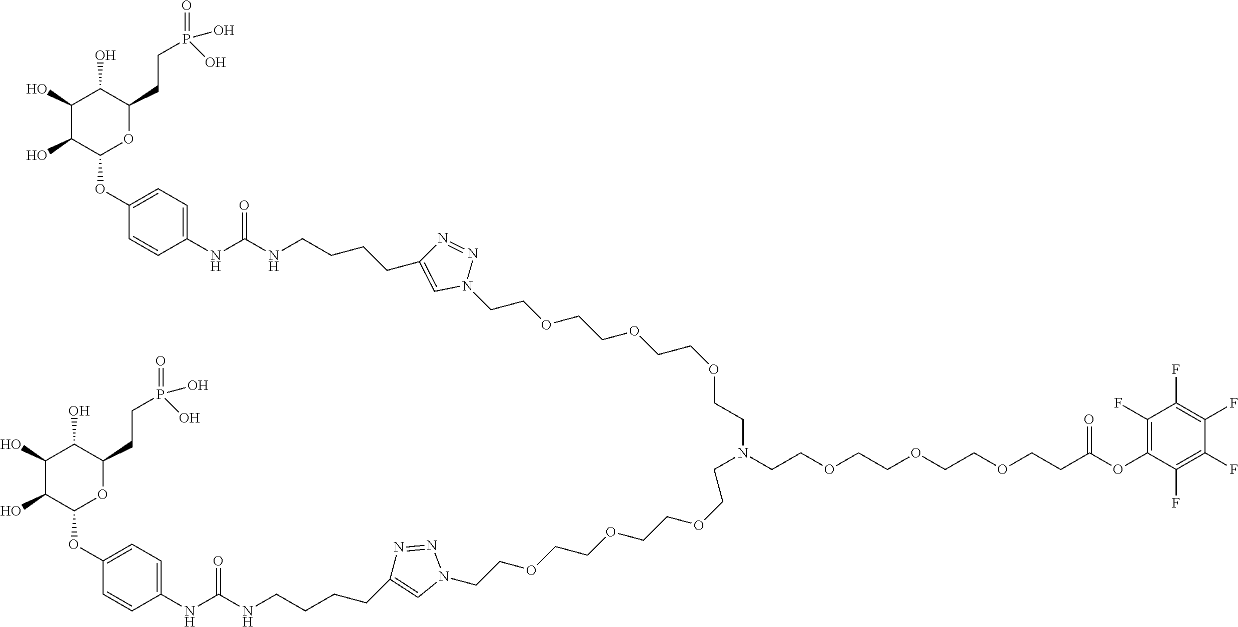 [SMILES: O=C(NCCCCc1cn(CCOCCOCCOCCN(CCOCCOCCOCCC(=O)Oc2c(F)c(F)c(F)c(F)c2F)CCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)nn1)Nc1ccc(O[C@H]2O[C@H](CCP(=O)(O)O)[C@@H](O)[C@H](O)[C@@H]2O)cc1] |
| 710 (I-53) | 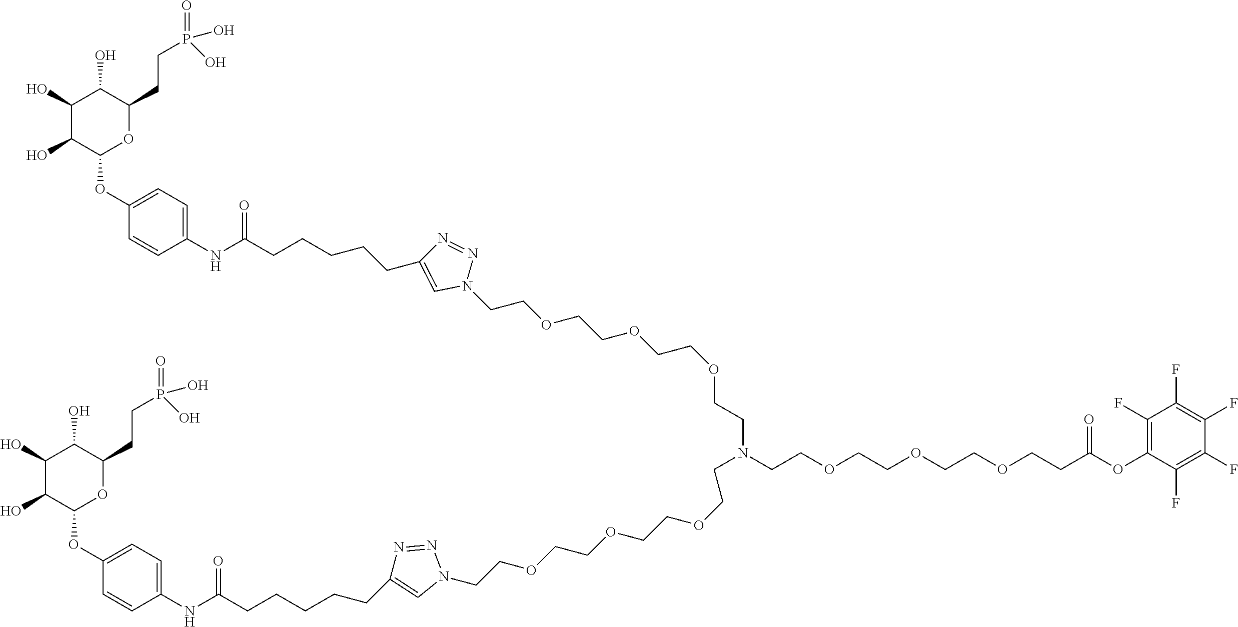 [SMILES: O=C(CCCCCc1cn(CCOCCOCCOCCN(CCOCCOCCOCCC(=O)Oc2c(F)c(F)c(F)c(F)c2F)CCOCCOCCOCCn2cc(CCCCCC(=O)Nc3ccc(O[C@H]4O[C@H](CCP(=O)(O)O)[C@@H](O)[C@H](O)[C@@H]4O)cc3)nn2)nn1)Nc1ccc(O[C@H]2O[C@H](CCP(=O)(O)O)[C@@H](O)[C@H](O)[C@@H]2O)cc1] |
| 711 (I-96) | 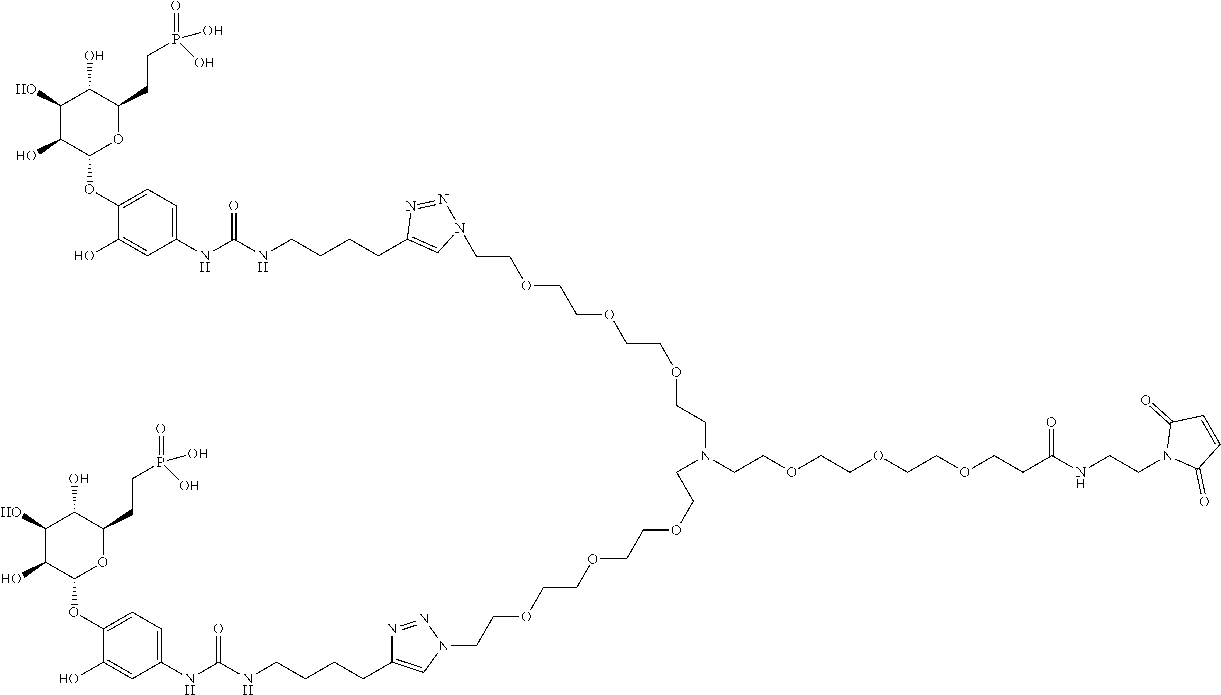 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)NCCN1C(=O)C=CC1=O] |
| 712 | 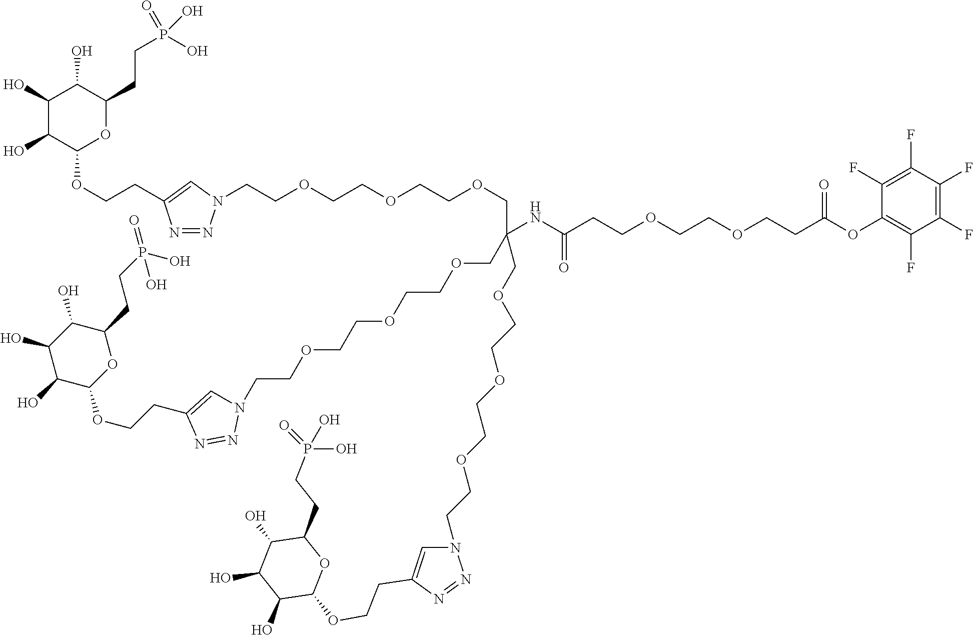 [SMILES: O=C(CCOCCOCCC(=O)Oc1c(F)c(F)c(F)c(F)c1F)NC(COCCOCCOCCn1cc(CCO[C@H]2O[C@H](CCP(=O)(O)O)[C@@H](O)[C@H](O)[C@@H]2O)nn1)(COCCOCCOCCn1cc(CCO[C@H]2O[C@H](CCP(=O)(O)O)[C@@H](O)[C@H](O)[C@@H]2O)nn1)COCCOCCOCCn1cc(CCO[C@H]2O[C@H](CCP(=O)(O)O)[C@@H](O)[C@H](O)[C@@H]2O)nn1] |
| 713 (I-44) | 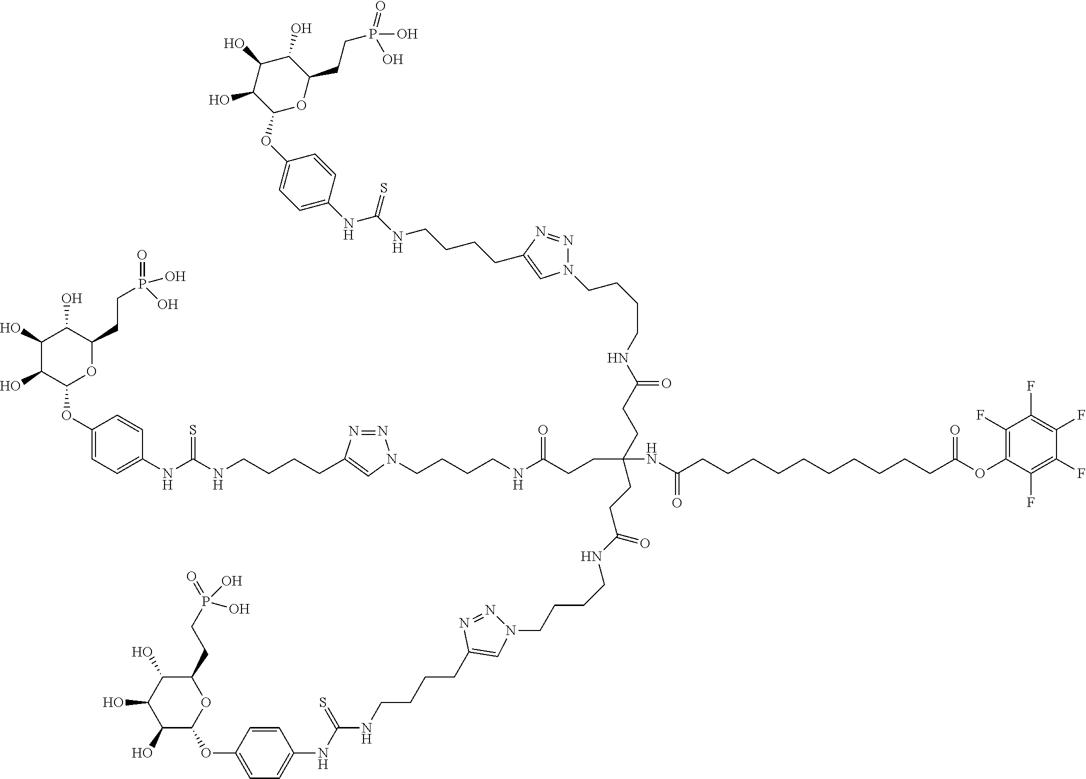 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCCCCCCCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 714 (I-45) | 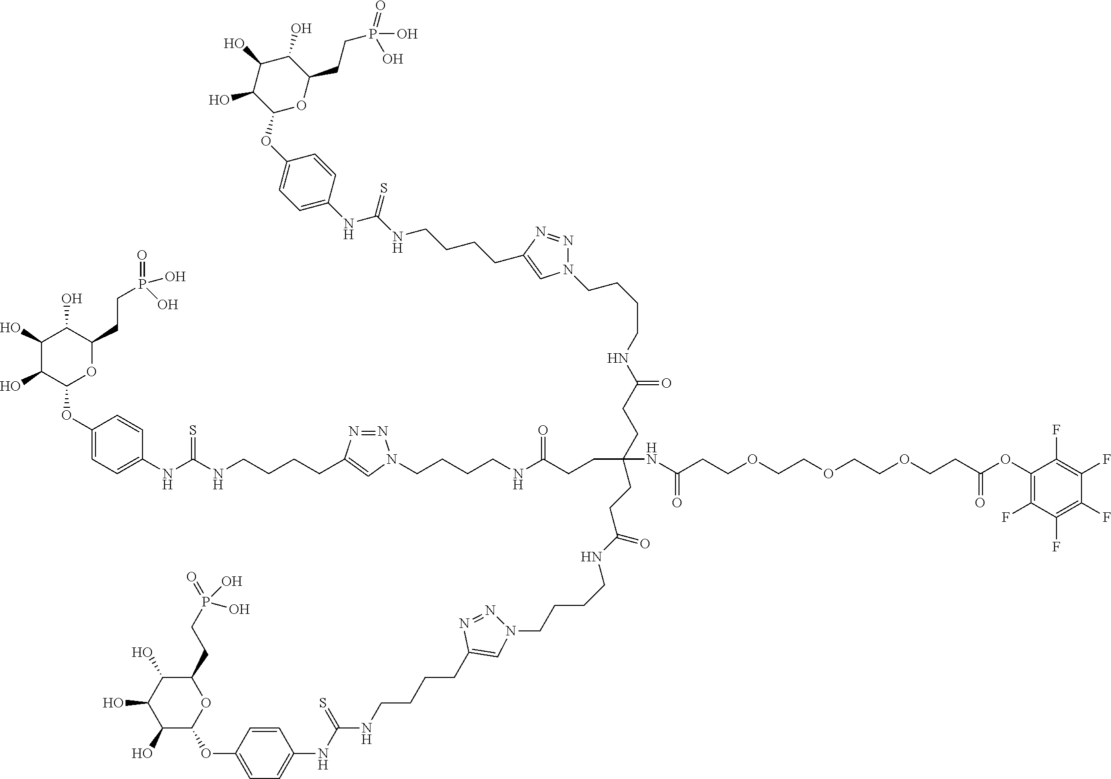 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCOCCOCCOCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 715 (I-54) | 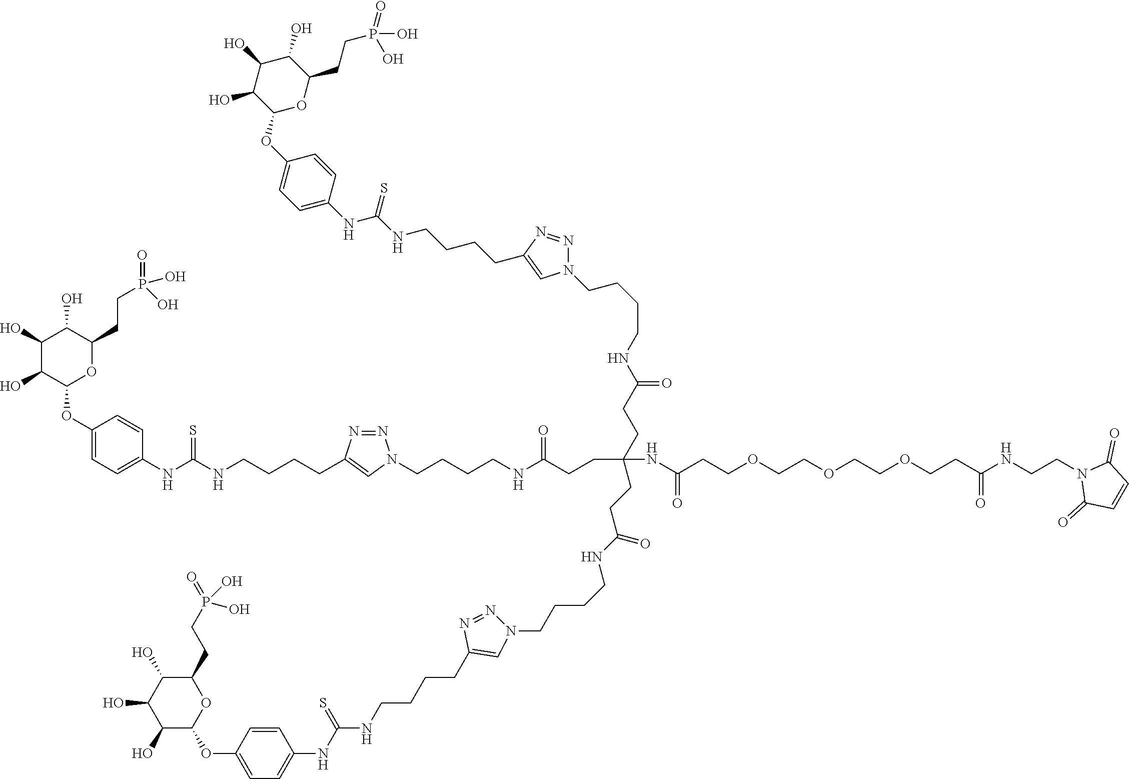 [SMILES: O=C(CCOCCOCCOCCC(=O)NC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 716 | 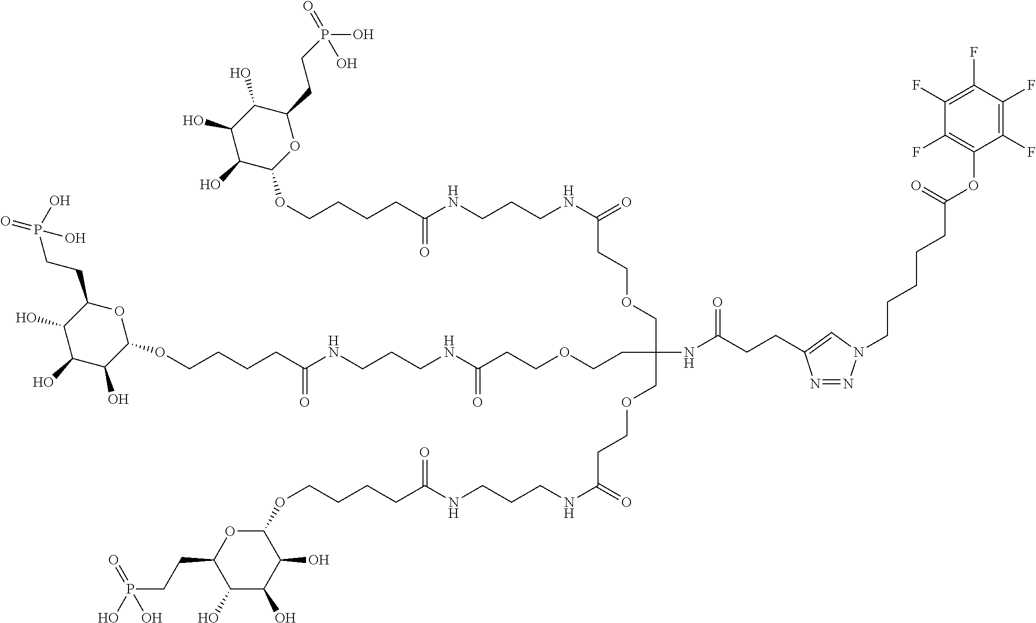 [SMILES: O=C(CCCCO[C@H]1O[C@H](CCP(=O)(O)O)[C@@H](O)[C@H](O)[C@@H]1O)NCCCNC(=O)CCOCCC(COCCC(=O)NCCCNC(=O)CCCCO[C@H]1O[C@H](CCP(=O)(O)O)[C@@H](O)[C@H](O)[C@@H]1O)(COCCC(=O)NCCCNC(=O)CCCCO[C@H]1O[C@H](CCP(=O)(O)O)[C@@H](O)[C@H](O)[C@@H]1O)NC(=O)CCc1cn(CCCCCC(=O)Oc2c(F)c(F)c(F)c(F)c2F)nn1] |
| 717 (I-81) | 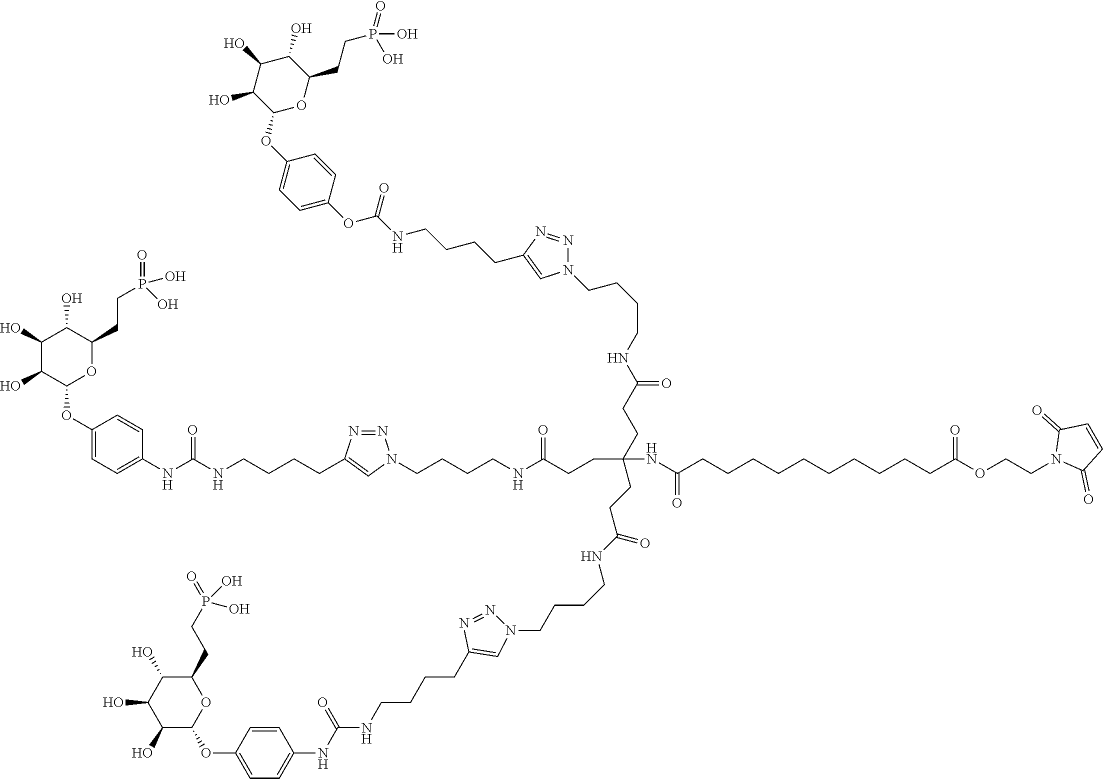 [SMILES: O=C(CCC(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=O)Oc2ccc(O[C@H]3O[C@H](CCP(=O)(O)O)[C@@H](O)[C@H](O)[C@@H]3O)cc2)nn1)NC(=O)CCCCCCCCCCC(=O)OCCN1C(=O)C=CC1=O)NCCCCn1cc(CCCCNC(=O)Nc2ccc(O[C@H]3O[C@H](CCP(=O)(O)O)[C@@H](O)[C@H](O)[C@@H]3O)cc2)nn1] |

In certain embodiments of formula (Ia), n is 2 or more (e.g., 3 or more, such as 3, 4, 5, or 6 or more) and the linker includes amino acid linking moieties that are branched and can be linked in a sequence together to provide for linkages via their sidechains (and optionally terminal groups) to multiple X ligands. In certain embodiments of formula (Ia), n is 3 or more, and Y is a chemoselective ligation group. In certain embodiments of formula (Ia), n is 4 or more, and Y is a chemoselective ligation group.

Exemplary multivalent compounds including amino acid residue linking moieties for use in preparing a bridging composition of this disclosure are shown in Table 10.

TABLE 10
Exemplary multivalent compounds including amino acid linking moieties

| # | Structure |
|---|---|
| 716 | 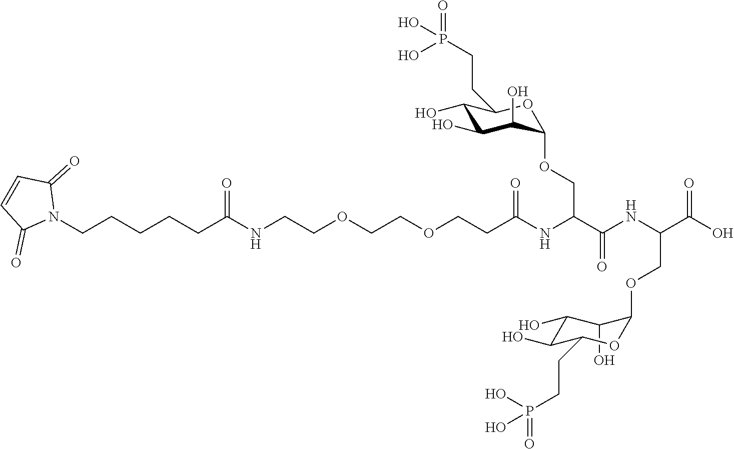 |
| 717 | 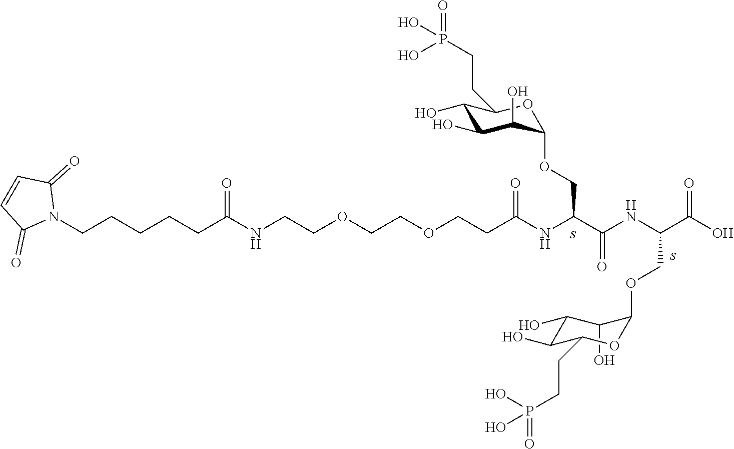 |
| 718 | 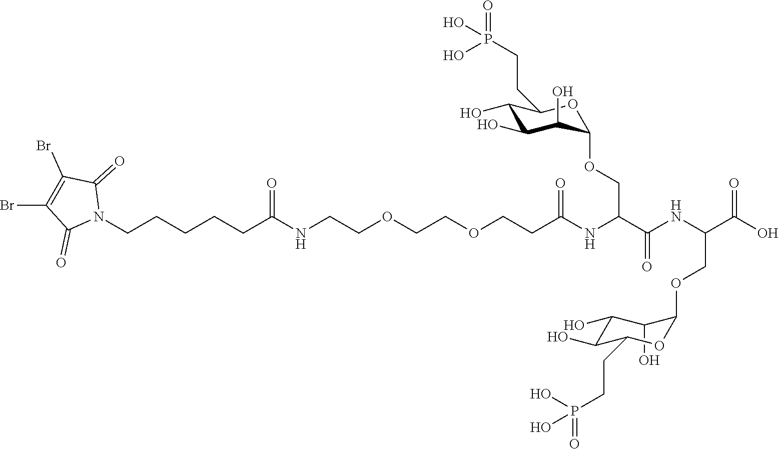 |
| 719 | 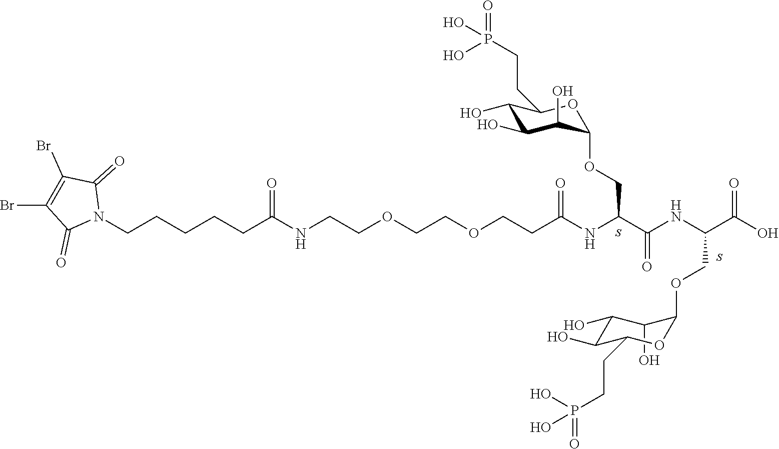 |
| 720 | 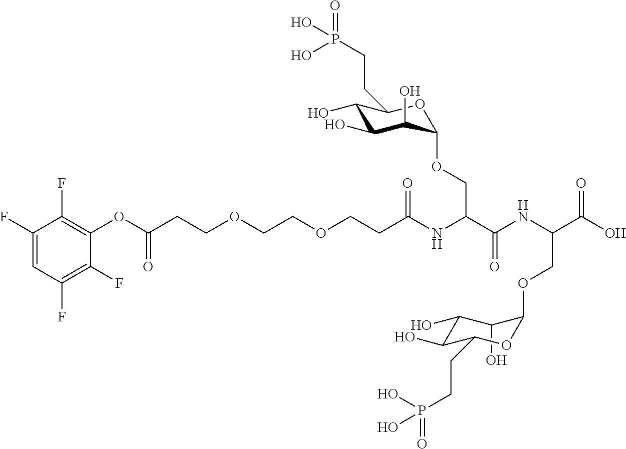 |
| 721 | 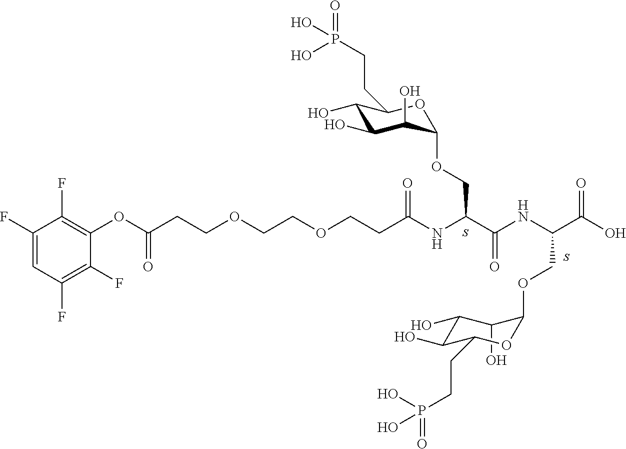 |
| 722 | 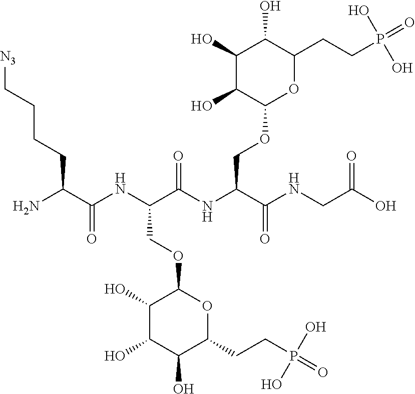 [SMILES: [N-]=[N+]=NCCCC[C@H](N)C(=O)N[C@@H](CO[C@H]1O[C@H](CCP(=O)(O)O)[C@@H](O)[C@H](O)[C@@H]1O)C(=O)N[C@@H](CO[C@H]1OC(CCP(=O)(O)O)[C@@H](O)[C@H](O)[C@@H]1O)C(=O)NCC(=O)O] |
| 723 (I-97) | 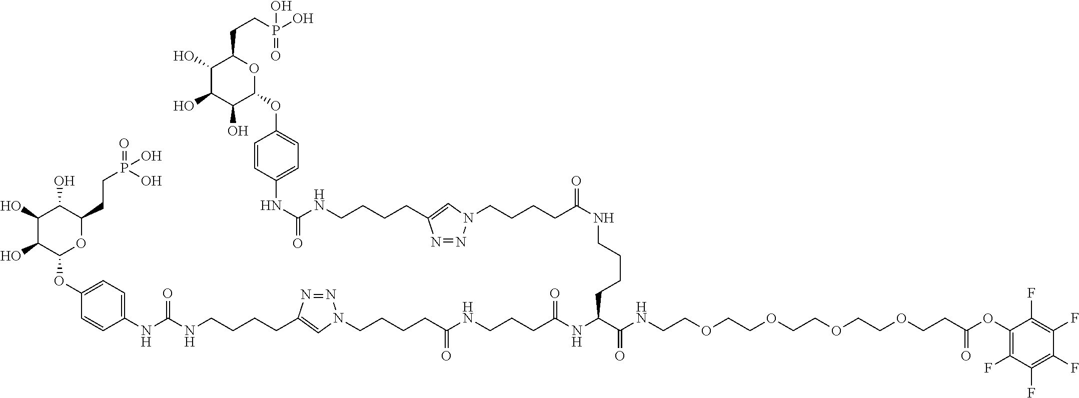 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 724 (I-98) | 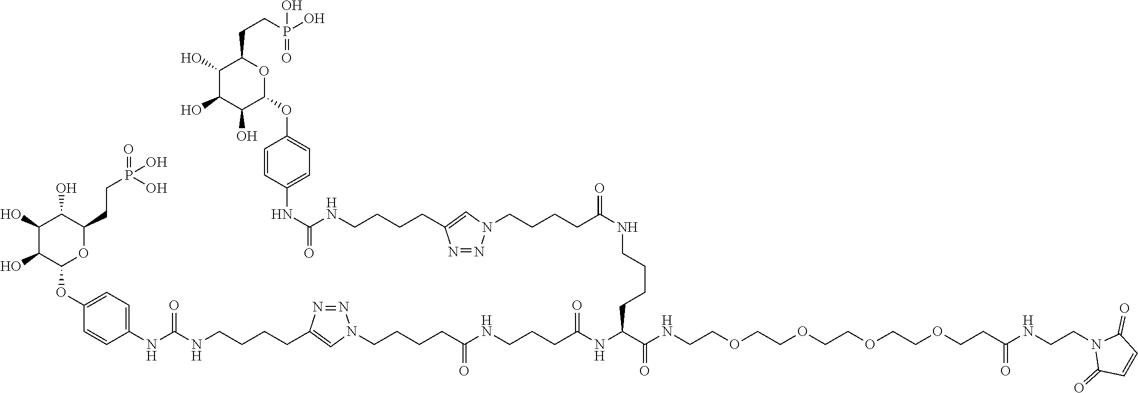 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| 725 (I-99) | 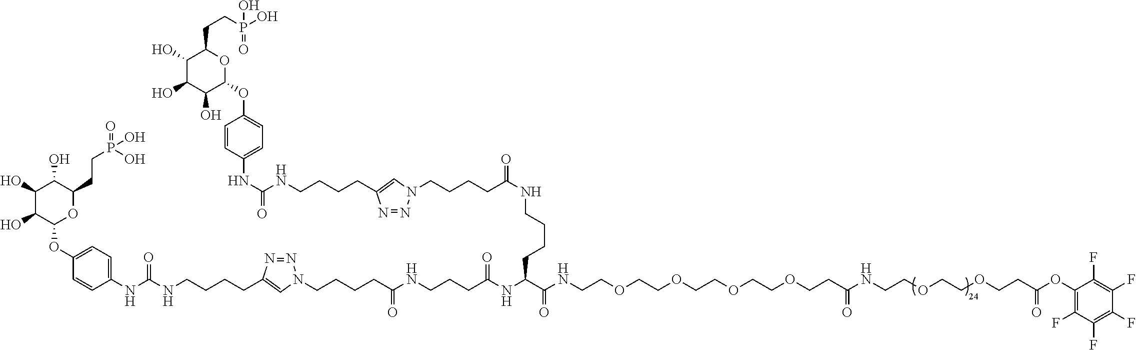 |
| 726 (I-100) | 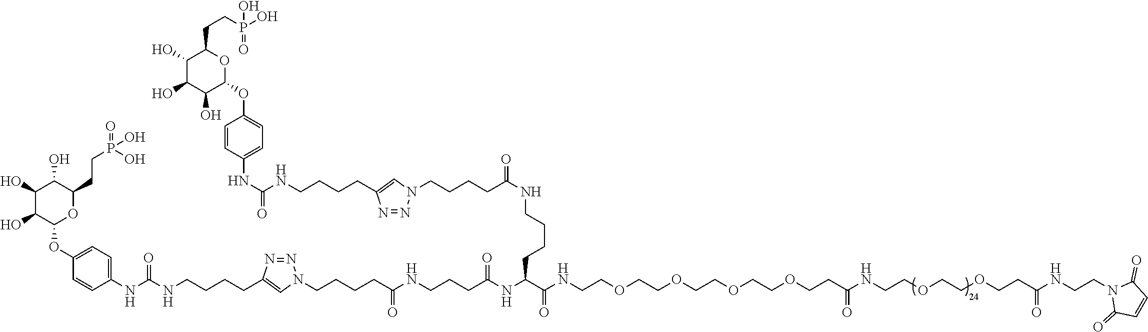 |
| 727 | 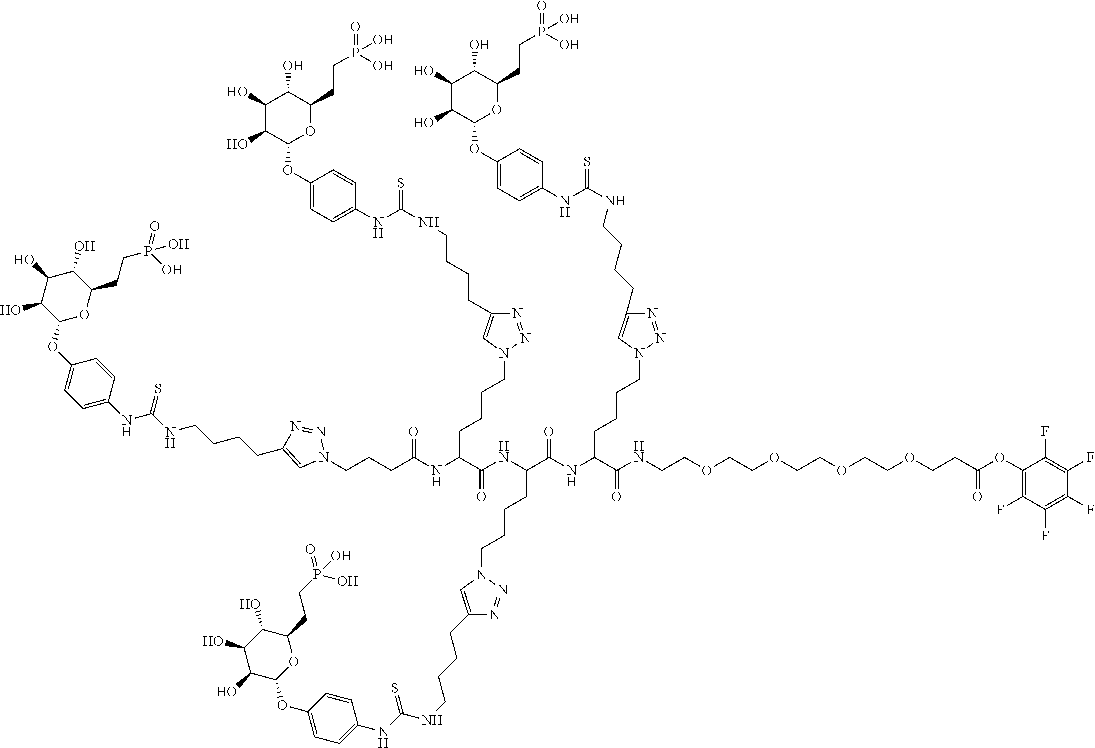 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 728 (I-52) | 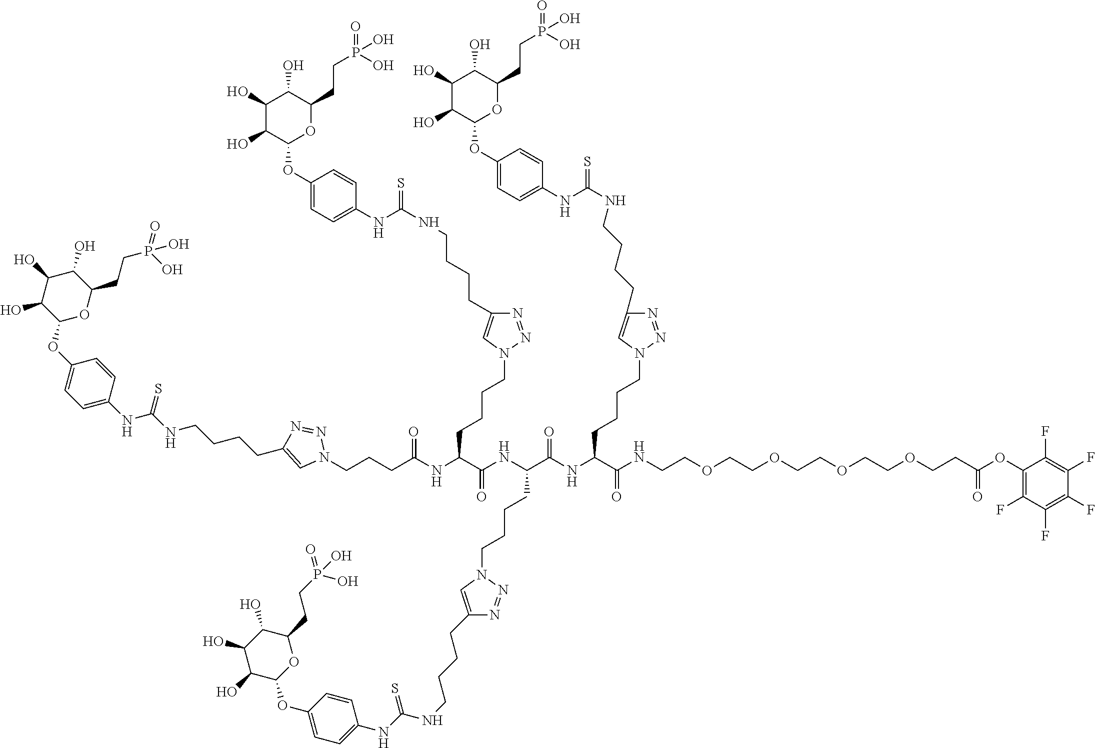 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 729 | 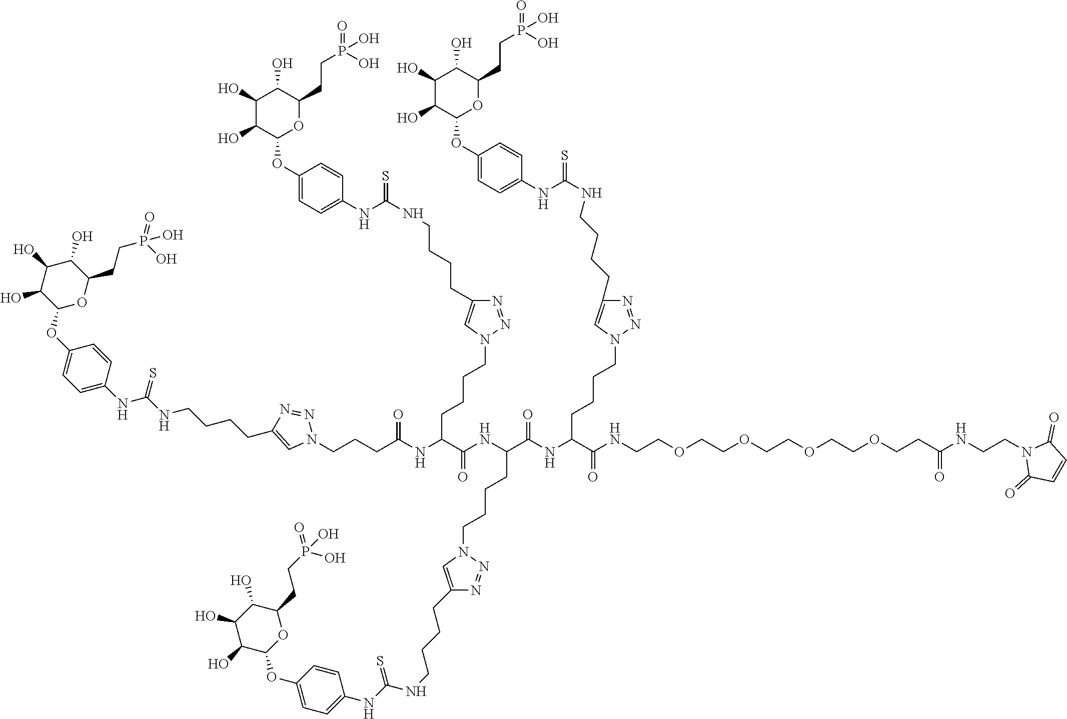 [SMILES: O=C(CCOCCOCCOCCOCCNC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 730 (I-56) | 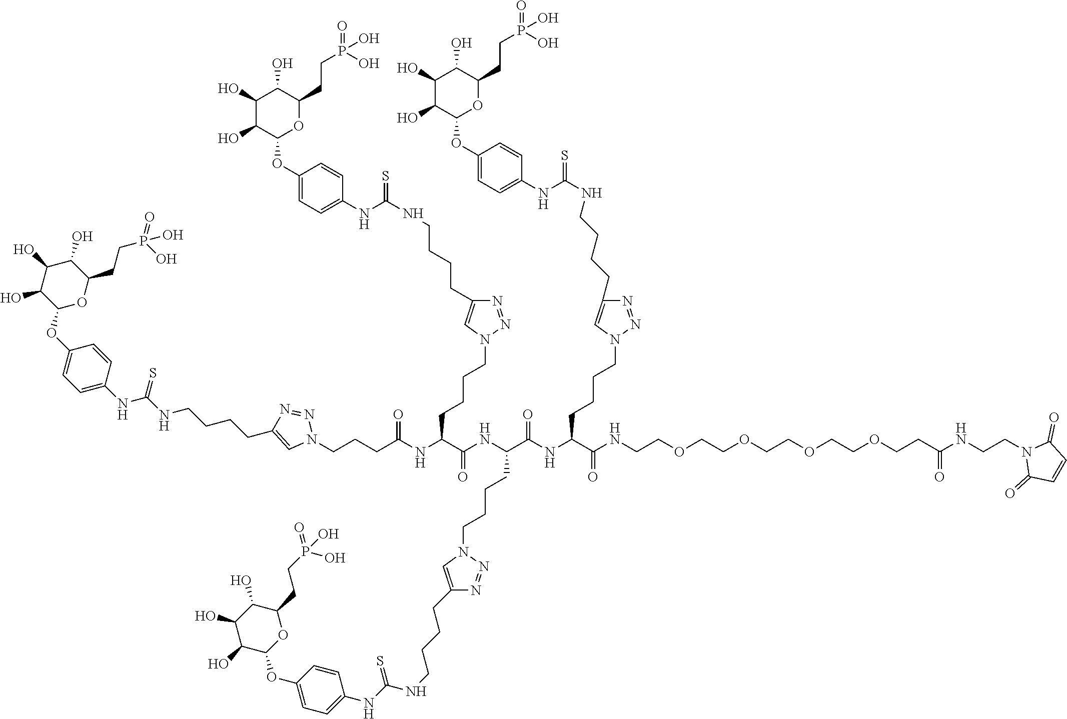 [SMILES: O=C(CCOCCOCCOCCOCCNC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 731 | 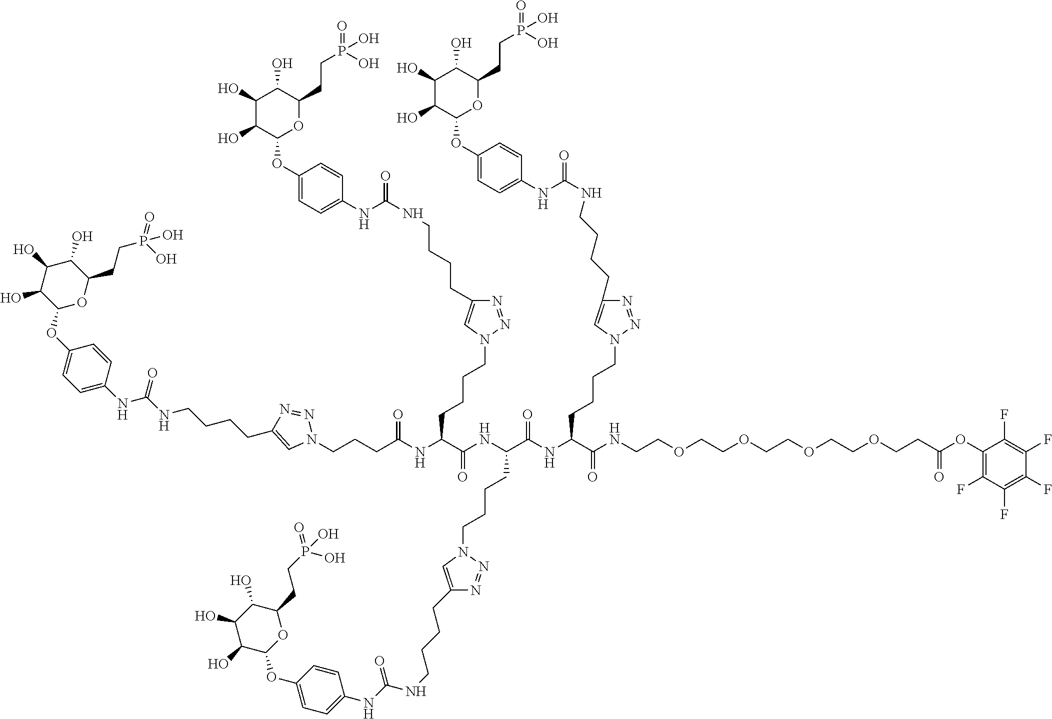 [SMILES: O=C(CCCn1cc(CCCCNC(=O)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 732 (I-82) | 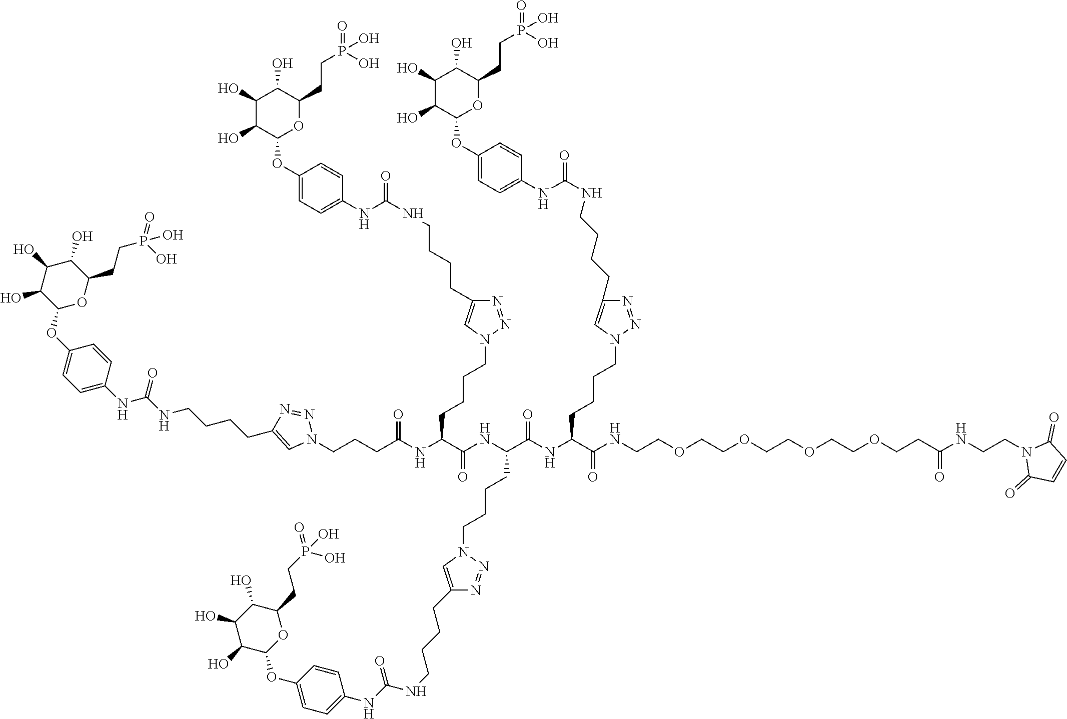 [SMILES: O=C(CCOCCOCCOCCOCCNC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)CCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |

The present disclosure is meant to encompass stereoisomers of any one of the compounds described herein. In some instance, the compound includes an enantiomer of the D-mannopyrannose ring, or analog thereof.

Exemplary ASGPR binding compounds of formula (Ib) are shown in Tables 11-12.

TABLE 11
ASGPR binding compounds of formula (Ib) and (IIIj)

| # | Structure |
|---|---|
| 801 (I-117) | 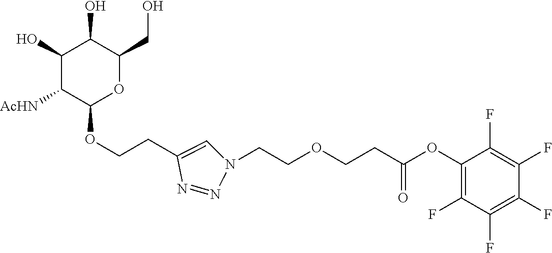 [SMILES: CC(=O)N[C@H]1[C@H](OCCc2cn(CCOCCC(=O)Oc3c(F)c(F)c(F)c(F)c3F)nn2)O[C@H](CO)[C@H](O)[C@@H]1O] |
| 802 (I-115) | 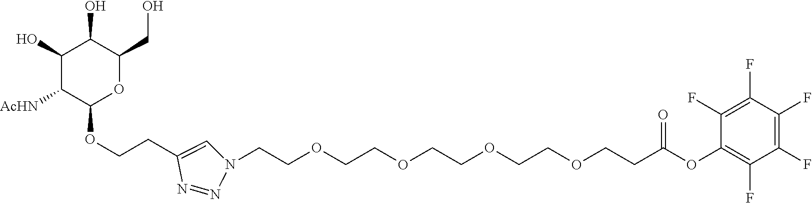 [SMILES: CC(=O)N[C@H]1[C@H](OCCc2cn(CCOCCOCCOCCOCCC(=O)Oc3c(F)c(F)c(F)c(F)c3F)nn2)O[C@H](CO)[C@H](O)[C@@H]1O] |
| 803 | 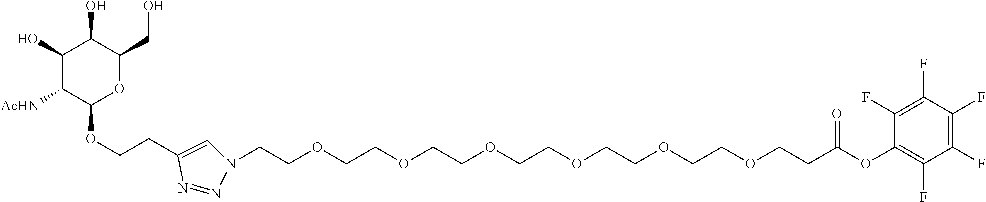 [SMILES: CC(=O)N[C@H]1[C@H](OCCc2cn(CCOCCOCCOCCOCCOCCOCCC(=O)Oc3c(F)c(F)c(F)c(F)c3F)nn2)O[C@H](CO)[C@H](O)[C@@H]1O] |
| 804 | 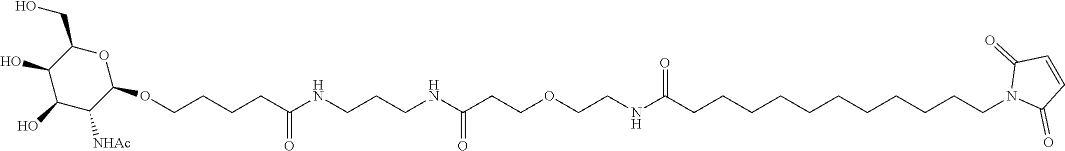 [SMILES: CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O] |
| 805 | 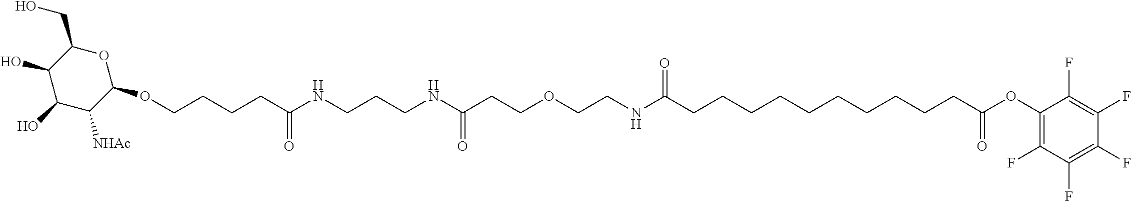 [SMILES: CC(=O)N[C@H]1[C@H](OCCCCC(=O)NCCCNC(=O)CCOCCNC(=O)CCCCCCCCCCC(=O)Oc2c(F)c(F)c(F)c(F)c2F)O[C@H](CO)[C@H](O)[C@@H]1O] |
| 806 (I-112) | 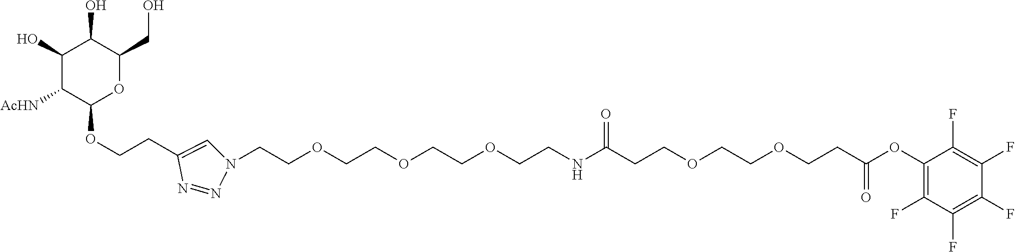 [SMILES: CC(=O)N[C@H]1[C@H](OCCc2cn(CCOCCOCCOCCNC(=O)CCOCCOCCC(=O)Oc3c(F)c(F)c(F)c(F)c3F)nn2)O[C@H](CO)[C@H](O)[C@@H]1O] |
| 807 | 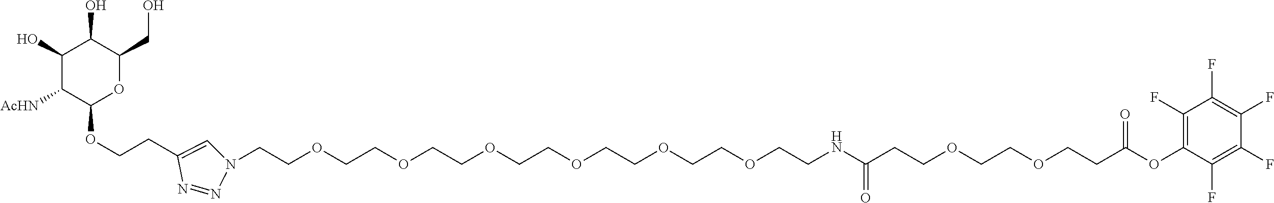 [SMILES: CC(=O)N[C@H]1[C@H](OCCc2cn(CCOCCOCCOCCOCCOCCOCCNC(=O)CCOCCOCCC(=O)Oc3c(F)c(F)c(F)c(F)c3F)nn2)O[C@H](CO)[C@H](O)[C@@H]1O] |
| 808 | 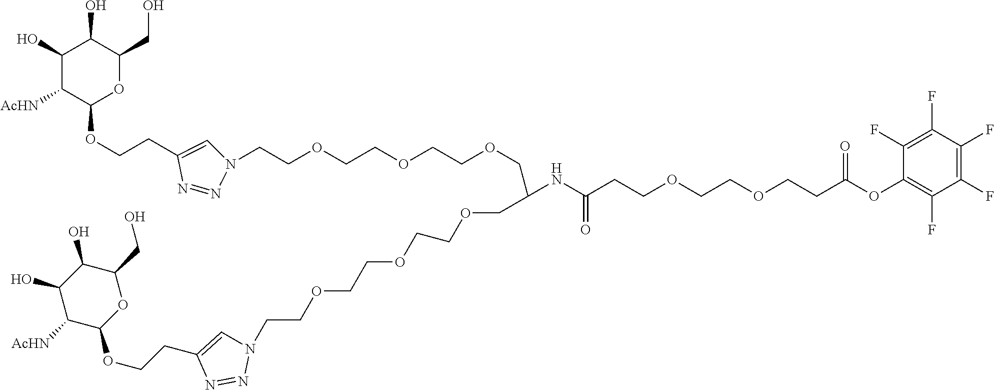 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 809 | 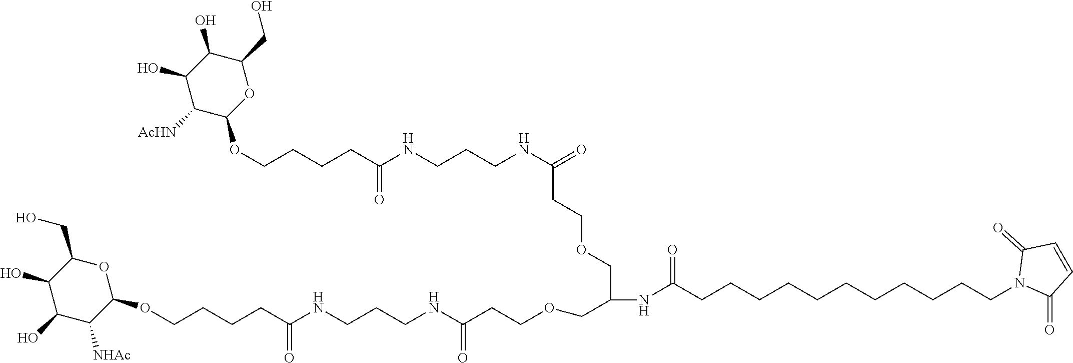 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 810 | 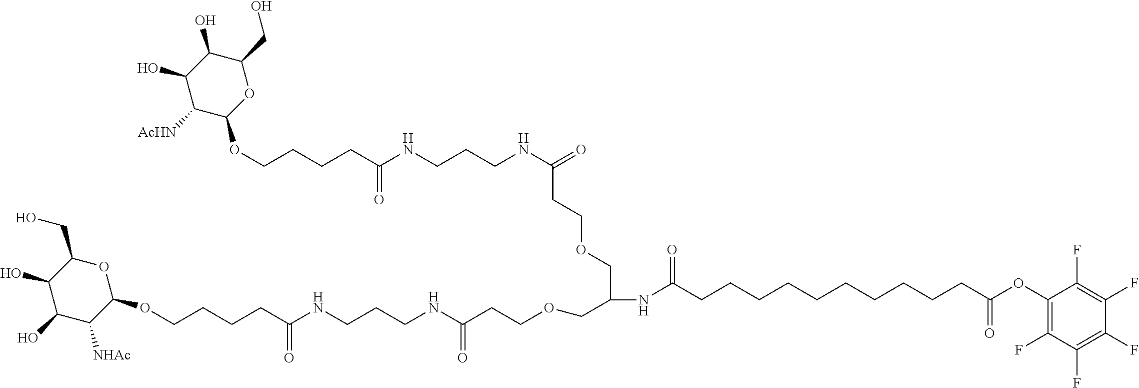 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 811 (I-111) | 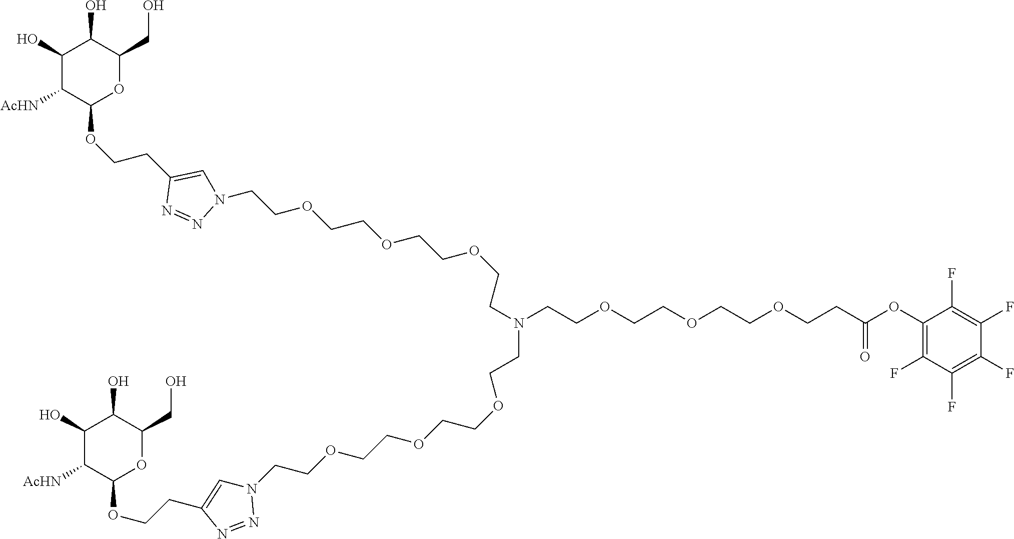 [SMILES: CC(=O)N[C@H]1[C@H](OCCc2cn(CCOCCOCCOCCN(CCOCCOCCOCCC(=O)Oc3c(F)c(F)c(F)c(F)c3F)CCOCCOCCOCCn3cc(CCO[C@@H]4O[C@H](CO)[C@H](O)[C@H](O)[C@H]4NC(C)=O)nn3)nn2)O[C@H](CO)[C@H](O)[C@@H]1O] |
| 812 (I-127) | 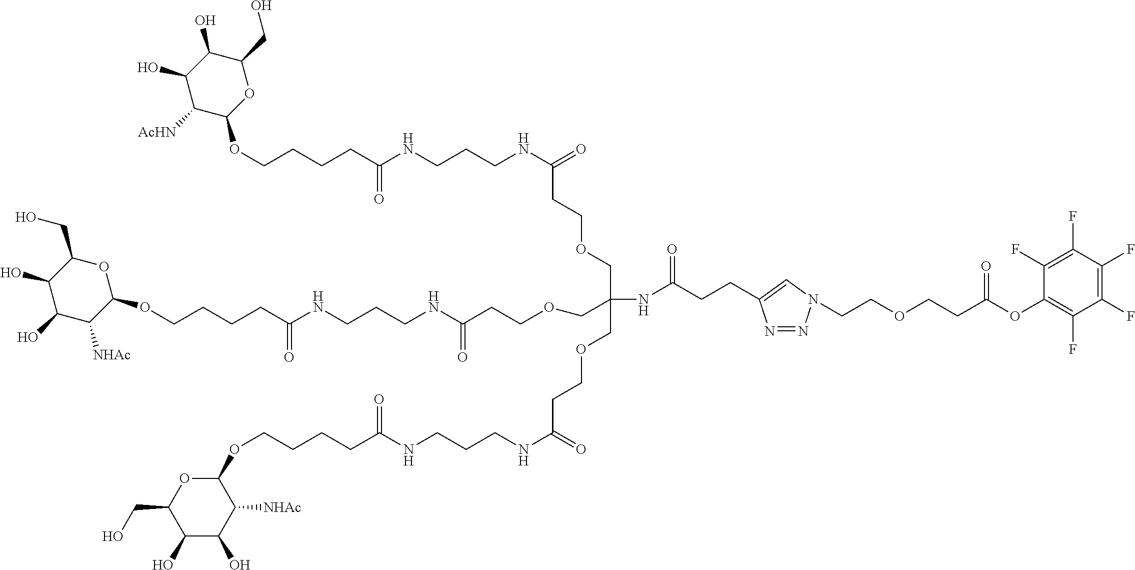 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCC(=O)Oc3c(F)c(F)c(F)c(F)c3F)nn2)O[C@H](CO)[C@H](O)[C@@H]1O] |
| 813 | 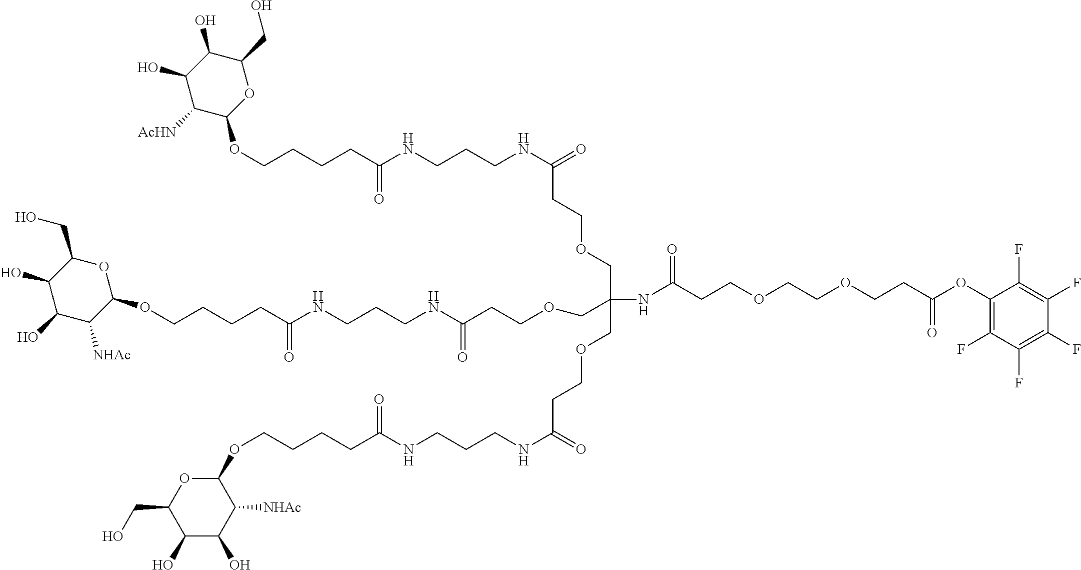 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCC(=O)Oc2c(F)c(F)c(F)c(F)c2F)O[C@H](CO)[C@H](O)[C@@H]1O] |
| 814 (I-107) | 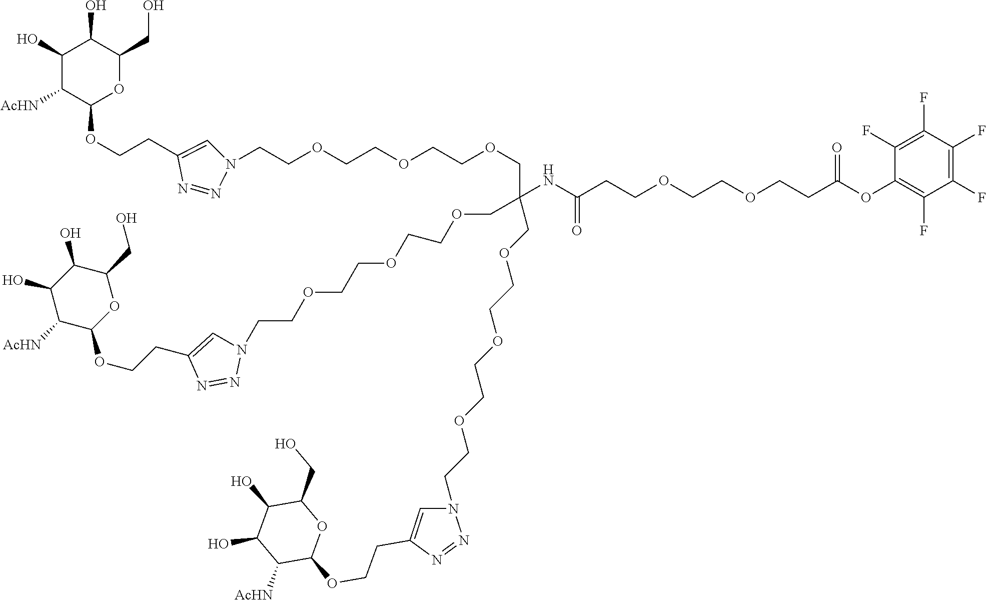 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 815 | 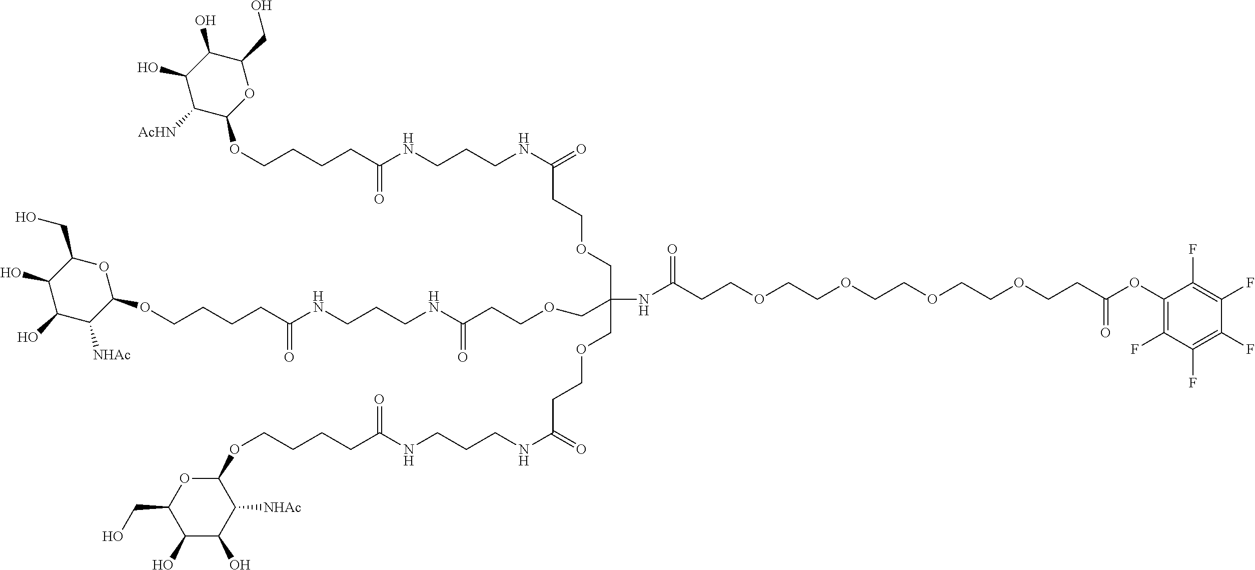 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCC(=O)Oc2c(F)c(F)c(F)c(F)c2F)O[C@H](CO)[C@H](O)[C@@H]1O] |
| 816 (I-124) | 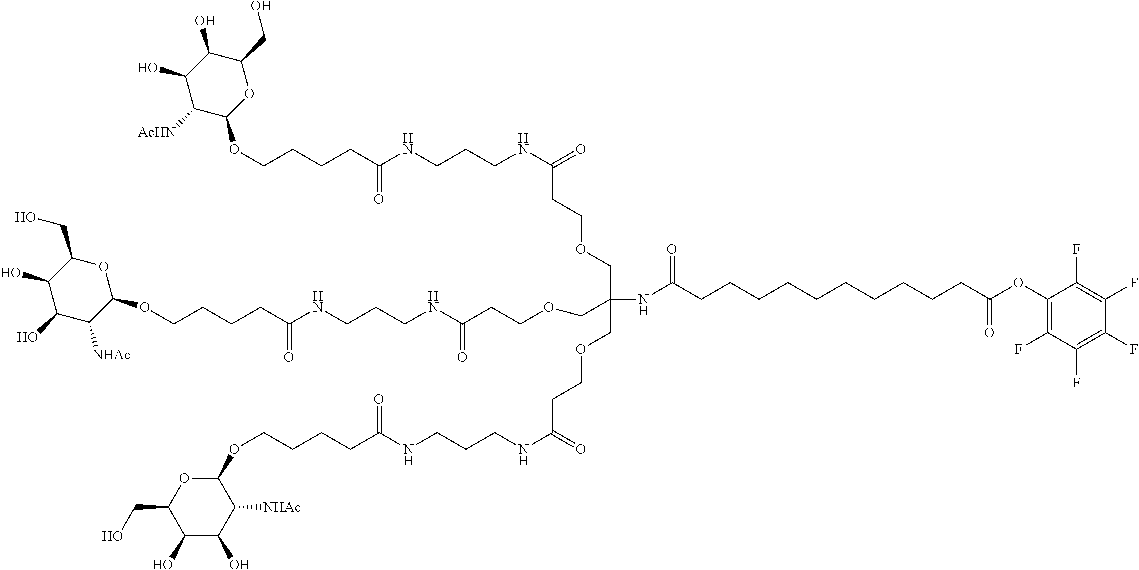 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 817 (I-123) | 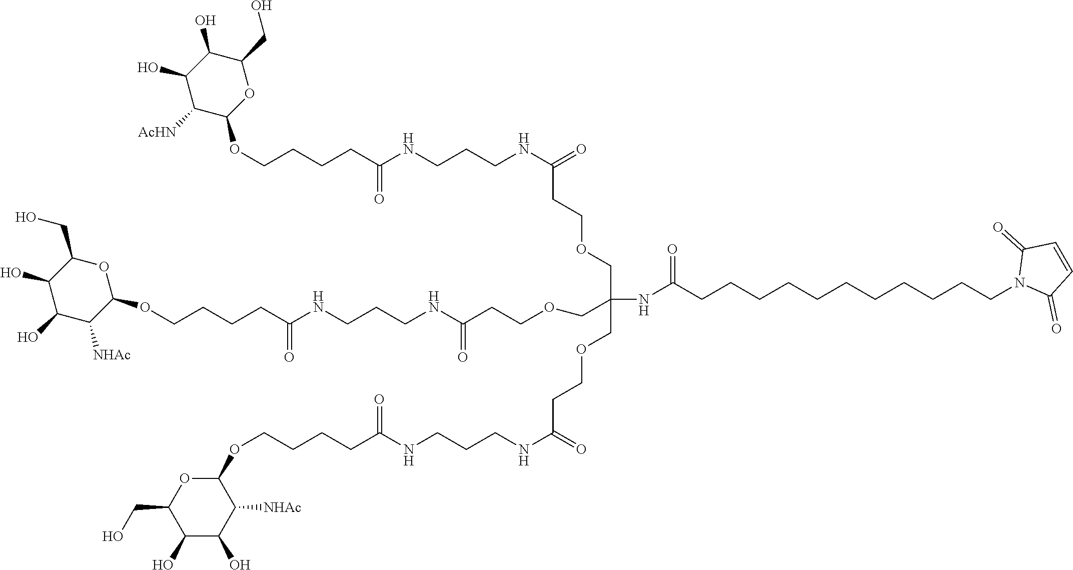 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 818 (I-129) | 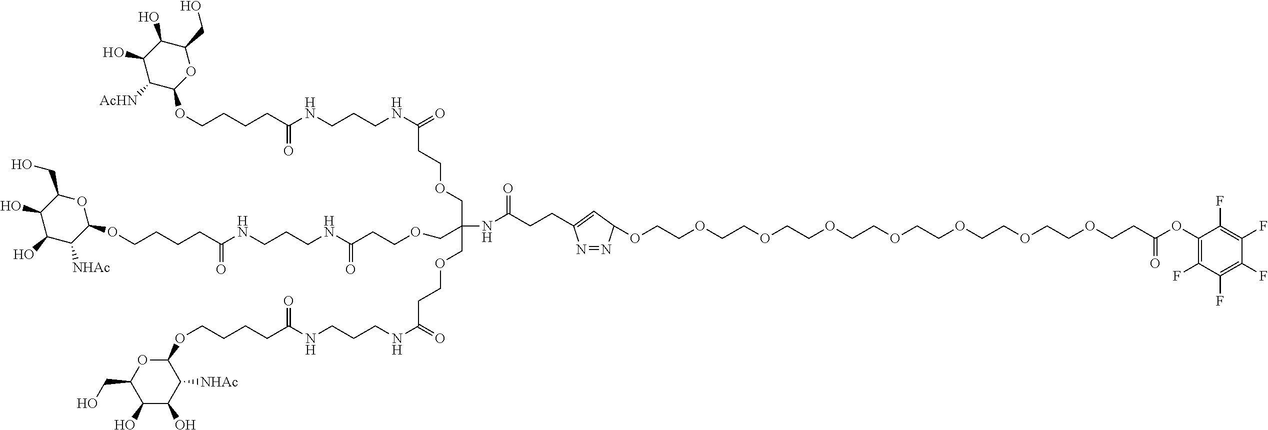 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCC2=CC(OCCOCCOCCOCCOCCOCCOCCOCCC(=O)Oc3c(F)c(F)c(F)c(F)c3F)N=N2)O[C@H](CO)[C@H](O)[C@@H]1O] |
| 819 | 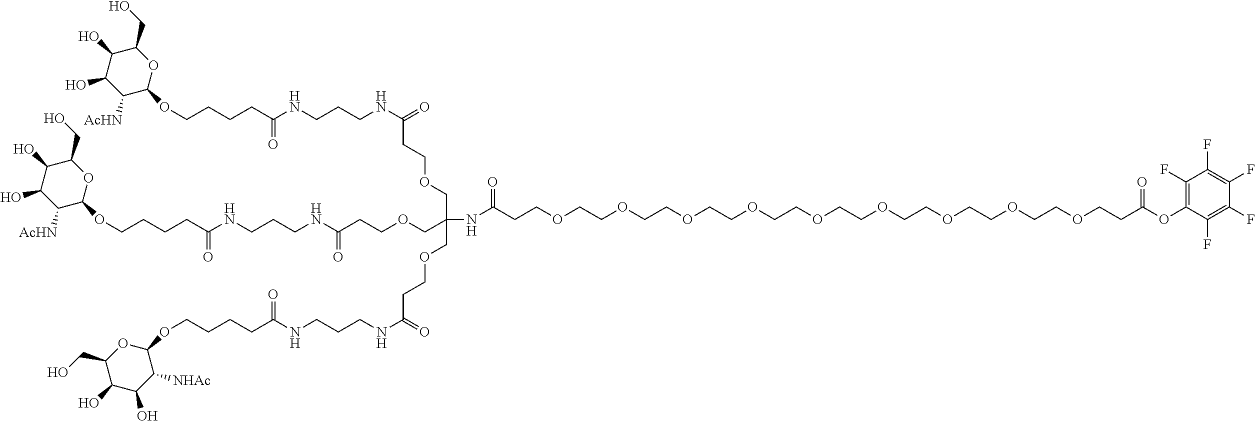 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCOCCOCCOCCOCCOCCC(=O)Oc2c(F)c(F)c(F)c(F)c2F)O[C@H](CO)[C@H](O)[C@@H]1O] |
| 820 | 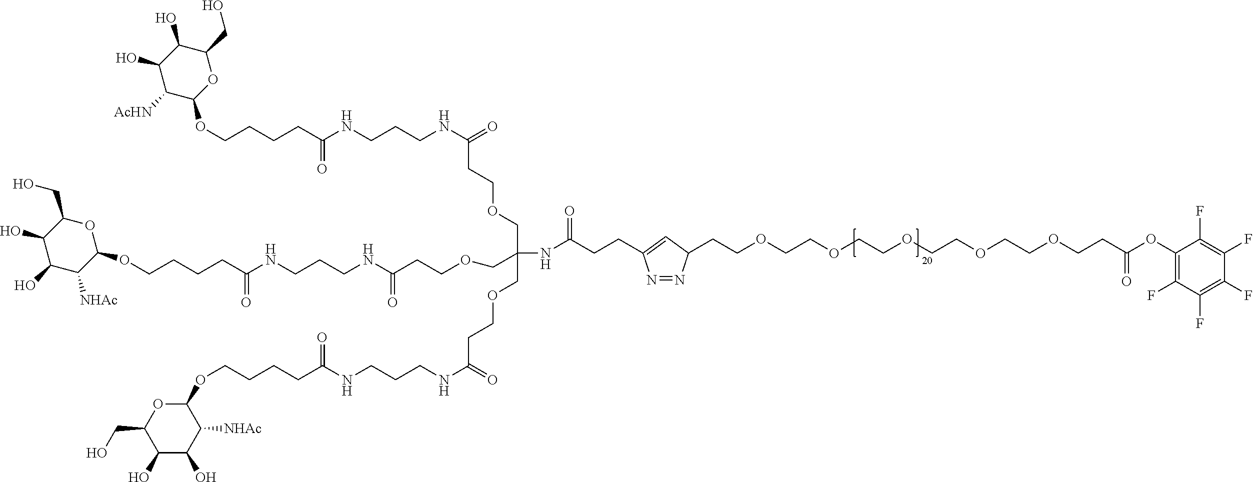 |
| 821 (I-135) | 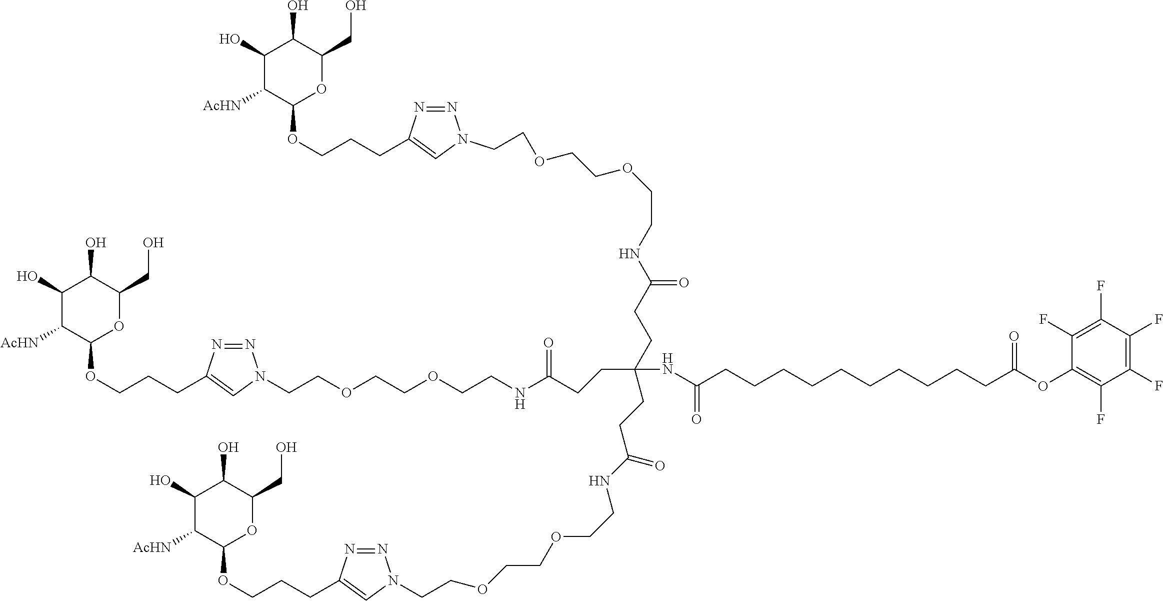 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](O)[C@H]4NC(C)=O)nn3)(CCC(=O)NCCOCCOCCn3cc(CCCO[C@@H]4O[C@H](CO)[C@H](O)[C@H](O)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1O] |

TABLE 12
ASGPR binding compounds of formula (Ib) and (IIIk)

| # | Structure |
|---|---|
| 901 (I-118) | 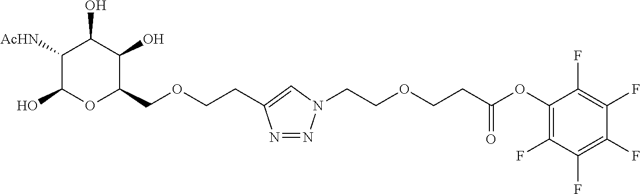 [SMILES: CC(=O)N[C@@H]1[C@@H](O)[C@@H](O)[C@@H](COCCc2cn(CCOCCC(=O)Oc3c(F)c(F)c(F)c(F)c3F)nn2)O[C@H]1O] |
| 902 (I-116) | 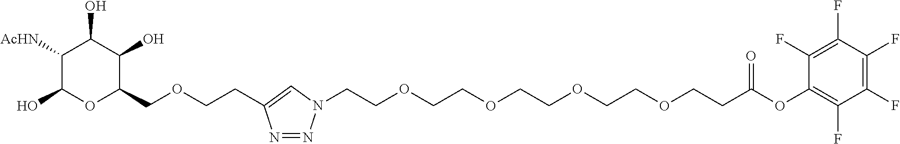 [SMILES: CC(=O)N[C@@H]1[C@@H](O)[C@@H](O)[C@@H](COCCc2cn(CCOCCOCCOCCOCCC(=O)Oc3c(F)c(F)c(F)c(F)c3F)nn2)O[C@H]1O] |
| 903 (I-113) | 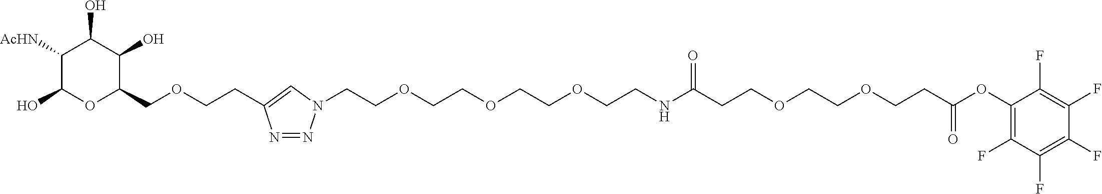 [SMILES: CC(=O)N[C@@H]1[C@@H](O)[C@@H](O)[C@@H](COCCc2cn(CCOCCOCCOCCNC(=O)CCOCCOCCC(=O)Oc3c(F)c(F)c(F)c(F)c3F)nn2)O[C@H]1O] |
| 904 (I-110) | 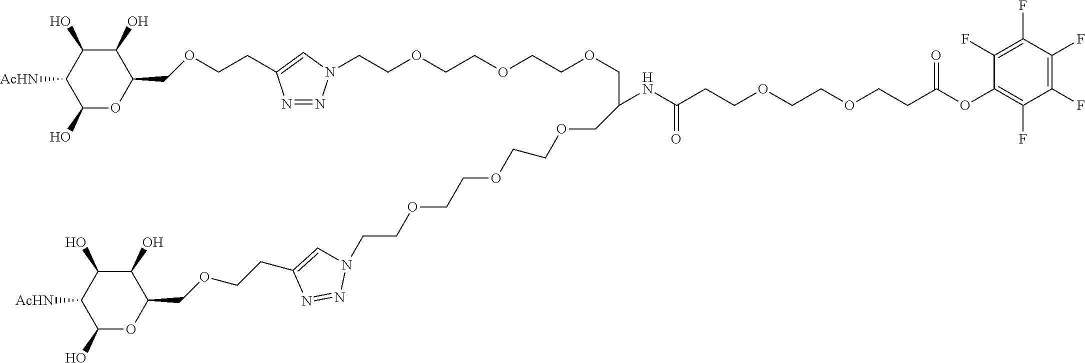 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 905 (I-108) | 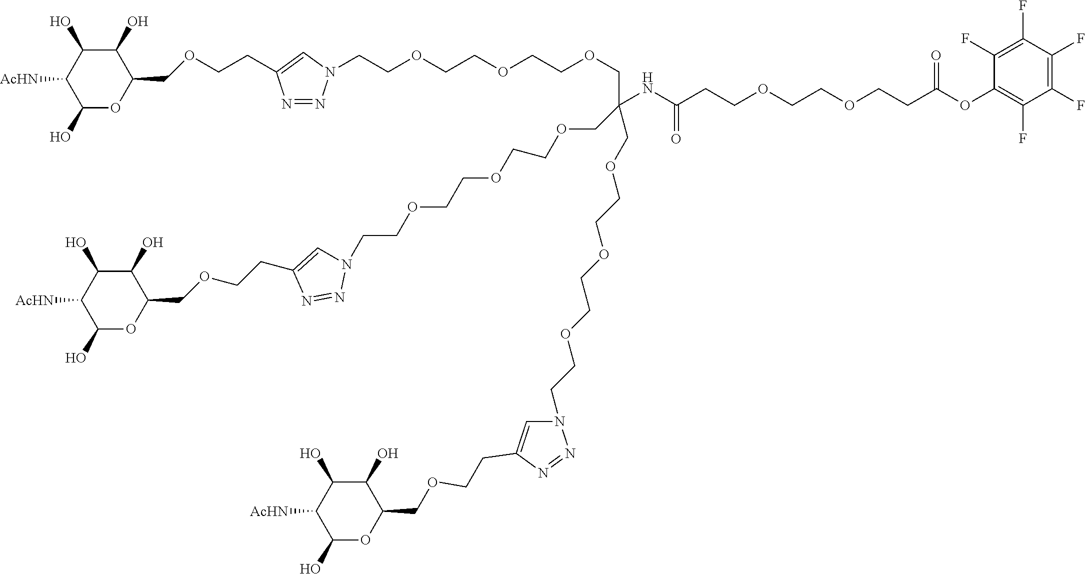 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 906 (I-136) | 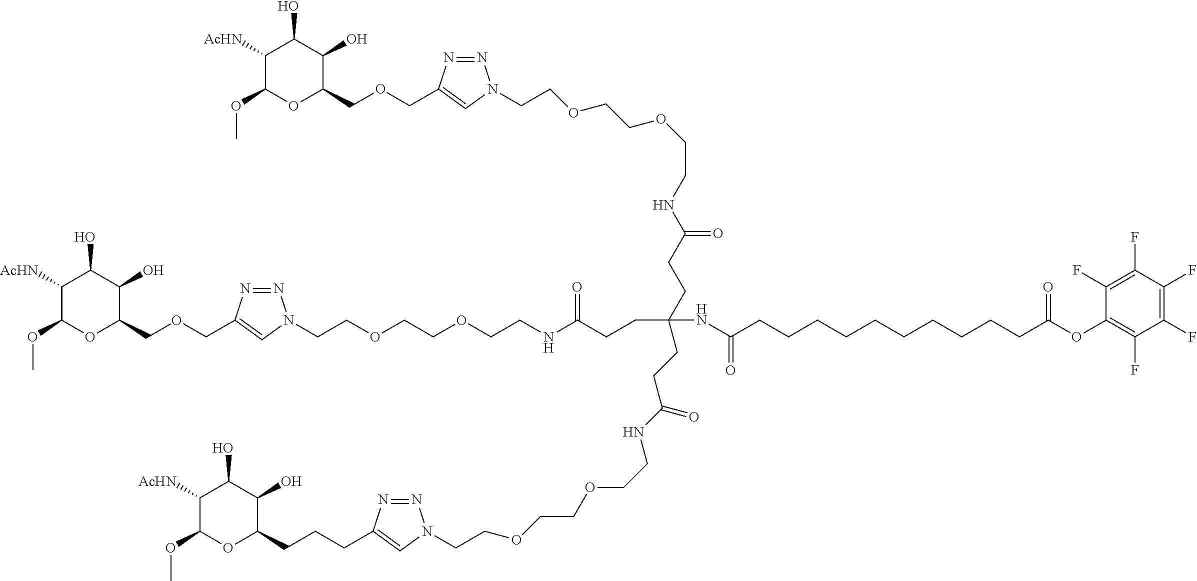 [SMILES: CO[C@@H]1O[C@H](CCCc2cn(CCOCCOCCNC(=O)CCC(CCC(=O)NCCOCCOCCn3cc(COC[C@H]4O[C@@H](OC)[C@H](NC(C)=O)[C@@H](O)[C@H]4O)nn3)(CCC(=O)NCCOCCOCCn3cc(COC[C@H]4O[C@@H](OC)[C@H](NC(C)=O)[C@@H](O)[C@H]4O)nn3)NC(=O)CCCCCCCCCCC(=O)Oc3c(F)c(F)c(F)c(F)c3F)nn2)[C@H](O)[C@H](O)[C@H]1NC(C)=O] |

In some embodiments, a folate receptor binding moiety-linker reagent that can be utilized to prepare a bridging composition of this disclosure is:

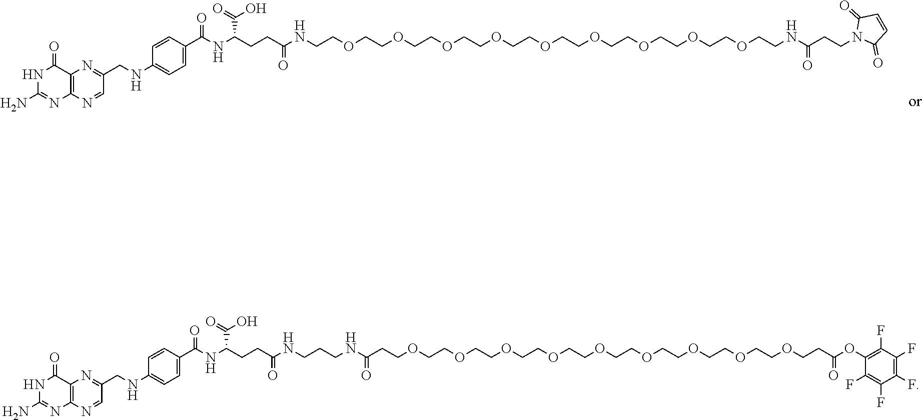 [SMILES: Nc1nc2ncc(CNc3ccc(C(=O)N[C@@H](CCC(=O)NCCOCCOCCOCCOCCOCCOCCOCCOCCOCCNC(=O)CCN4C(=O)C=CC4=O)C(=O)O)cc3)nc2c(=O)[nH]1 ; Nc1nc2ncc(CNc3ccc(C(=O)N[C@@H](CCC(=O)NCCCNC(=O)CCOCCOCCOCCOCCOCCOCCOCCOCCOCCC(=O)Oc4c(F)c(F)c(F)c(F)c4F)C(=O)O)cc3)nc2c(=O)[nH]1]

In another embodiment, a folate receptor binding moiety-linker reagent that can be utilized to prepare a bridging composition of this disclosure is selected from:

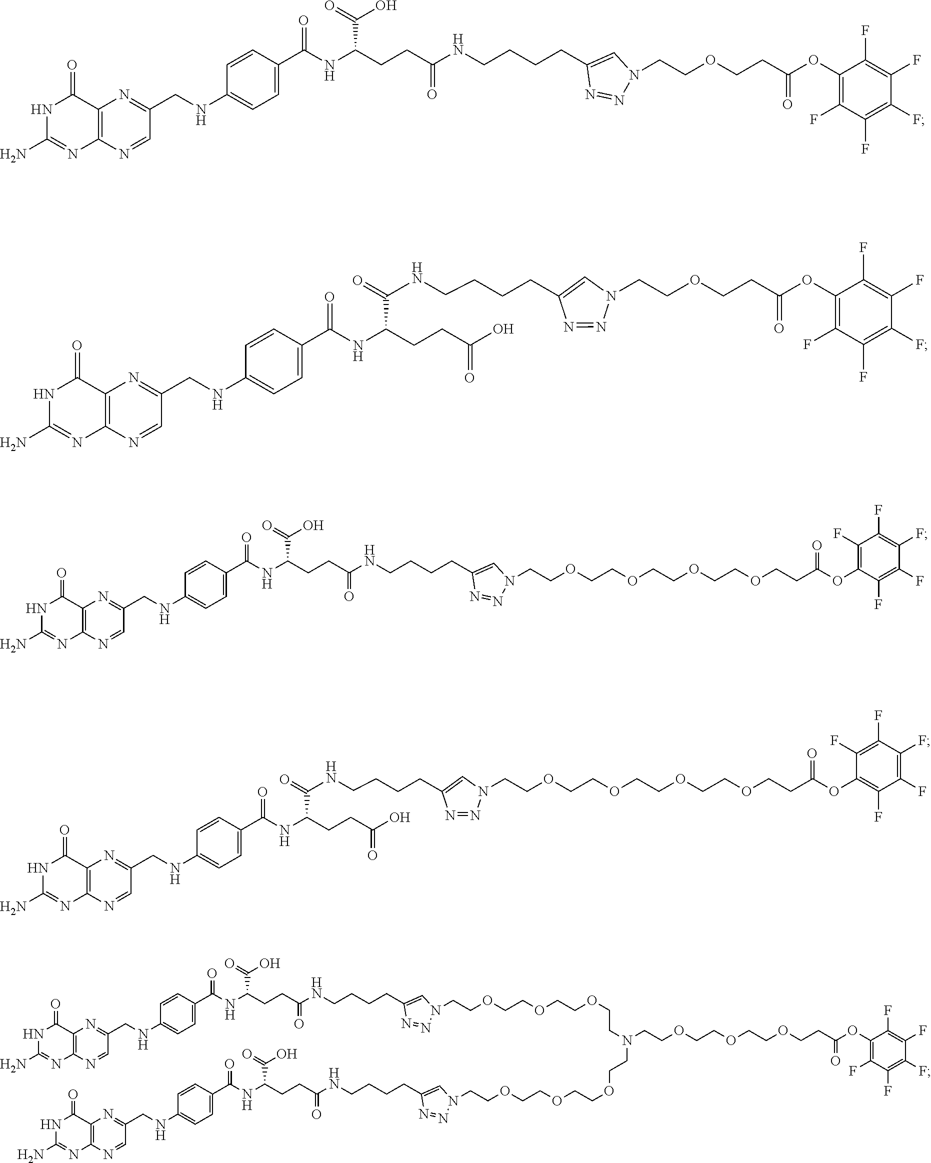

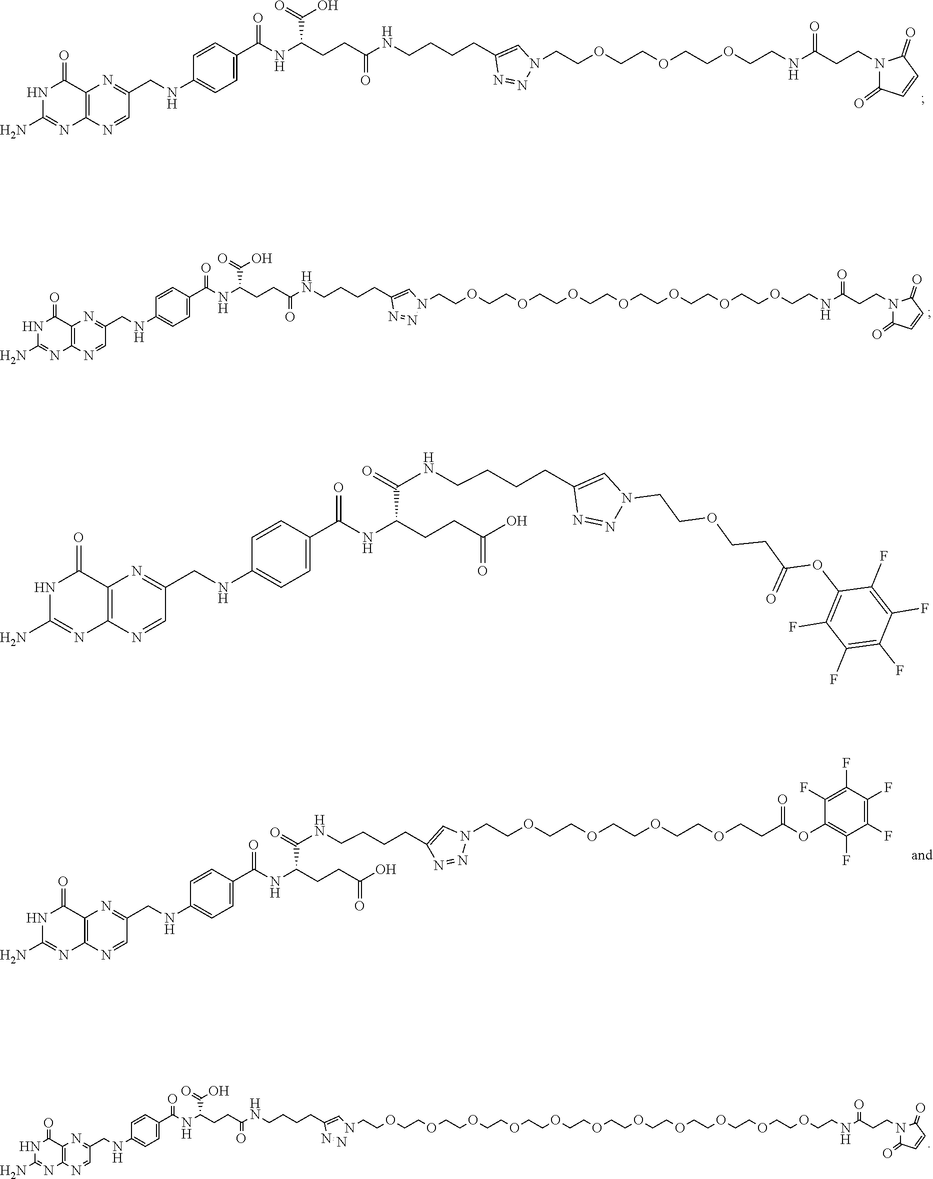

5.2.5. Exemplary Bridging Compositions

This disclosure includes bridging compositions which can include: (1) one or more particular M6PR ligand (X) (e.g., as described herein, such as ligands X1-X38 of Table 1) or a particular ASGPR ligand (X) (e.g., as described herein) or a particular folate receptor ligand (X) (e.g., as described herein), (2) a linker including one or more linking moieties (e.g., as described herein, such as any one or more of the linking moieties of Tables 8-9); and (3) a residual group produced by conjugation of a bridging moiety and a chemoselective ligation group (Y) e.g., as described herein, such as any one of the groups of Table 8) that has been conjugated to a bridging moiety. Exemplary bridging compositions are described herein and below.

In certain embodiments, the bridging composition of formulas (I)-(Ib) is selected from:

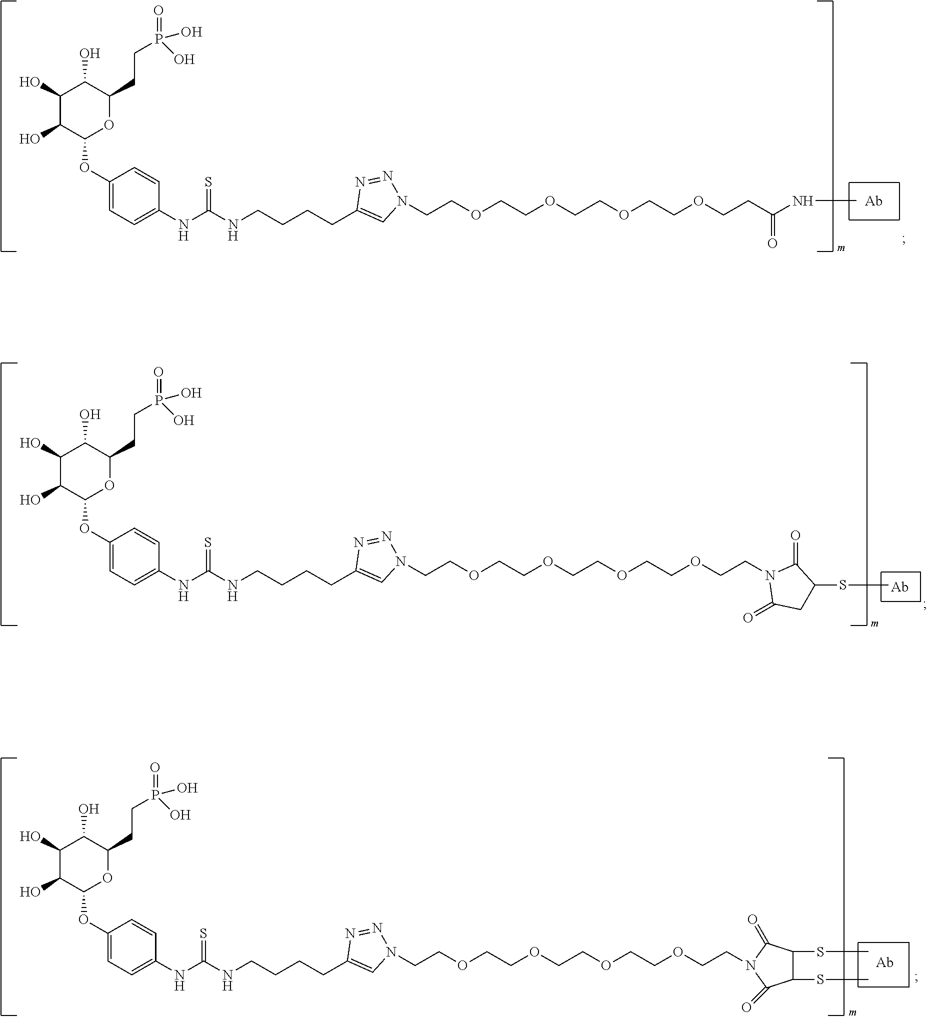

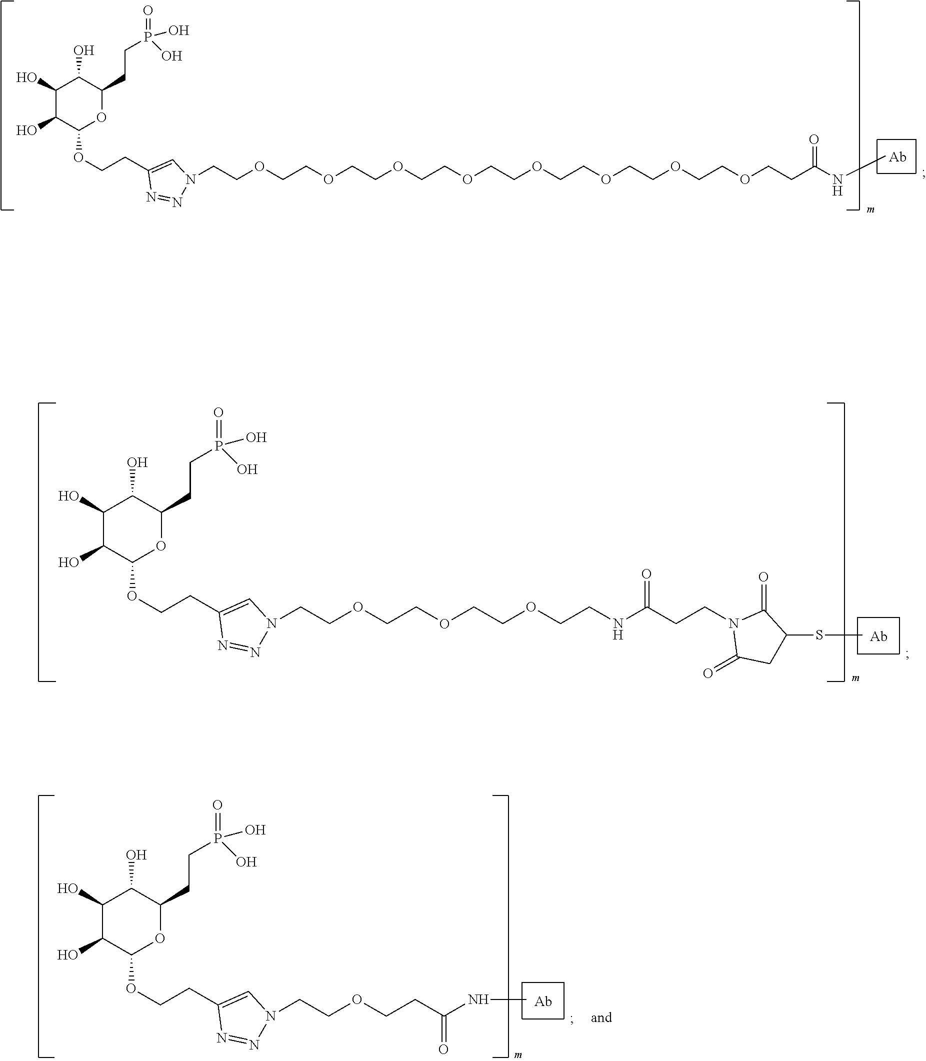

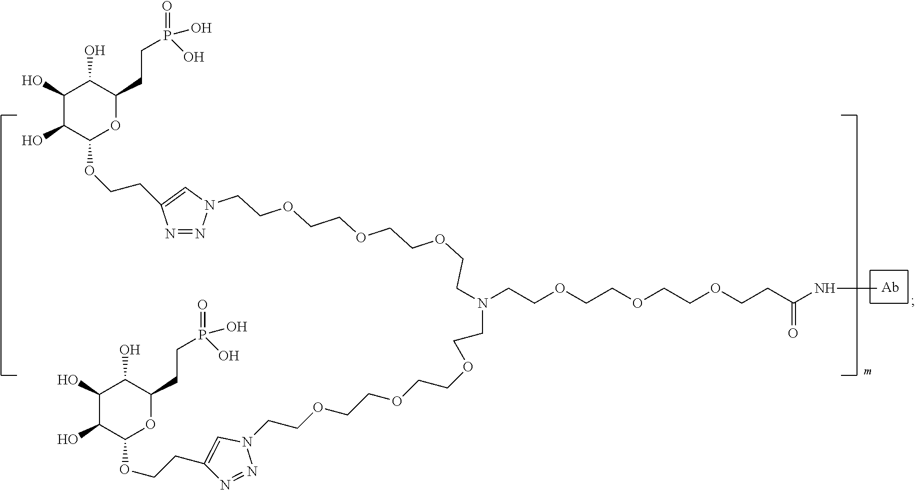

or a pharmaceutically acceptable salt thereof, wherein:
m is from 1 to 80; and

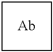

is an antibody or antibody fragment.

In certain embodiments, the bridging composition of formulas (I)-(Ib) is selected from:

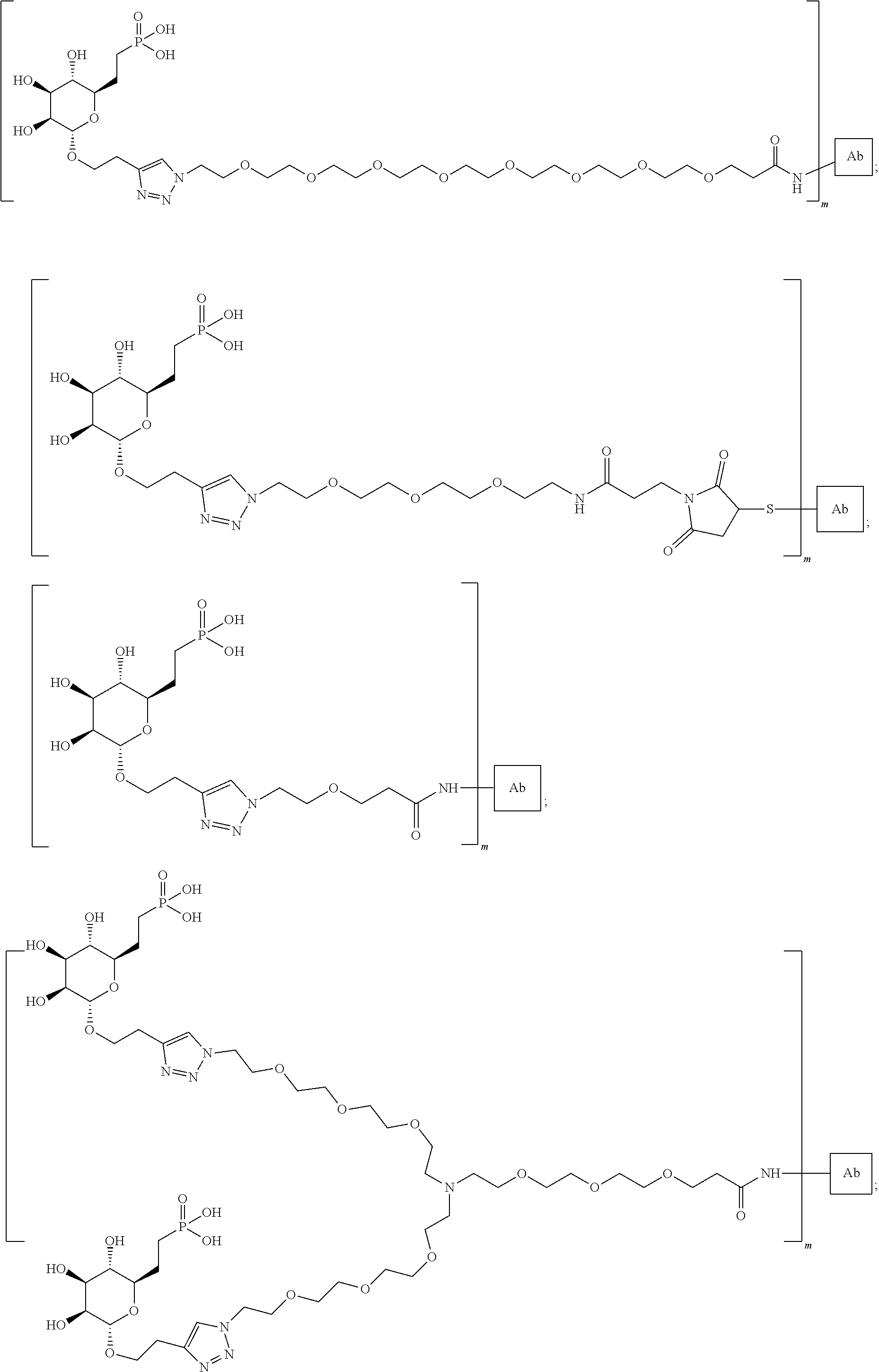

or a pharmaceutically acceptable salt thereof, wherein:
m is an integer from 1 to 80; and

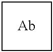

is an antibody or antibody fragment.

In certain embodiments, the bridging composition of formulas (I)-(Ib) is selected from:

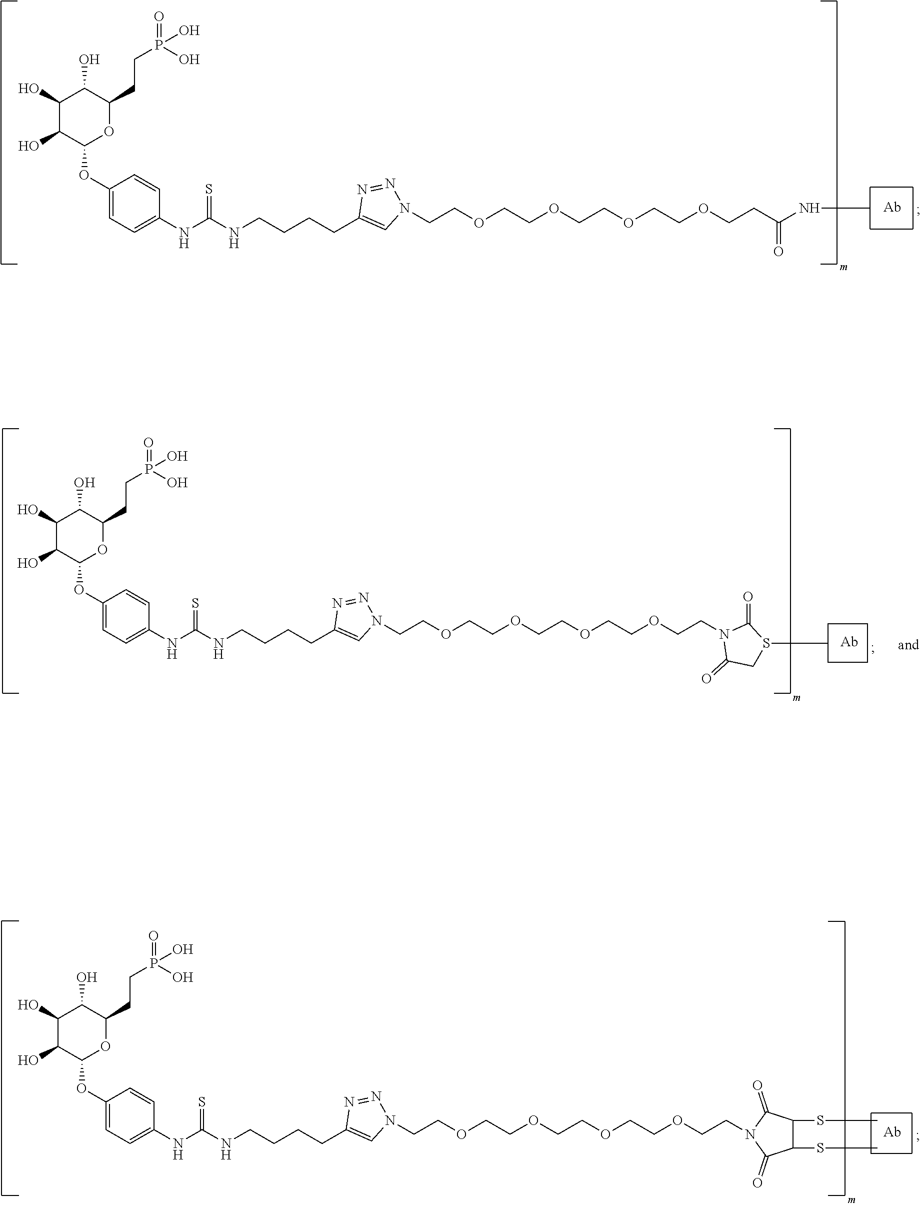

or a pharmaceutically acceptable salt thereof, wherein:
m is an integer from 1 to 80; and

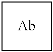

is an antibody or antibody fragment.

In certain embodiments, the bridging composition of formulas (I), (Ib) is of formula (IX):

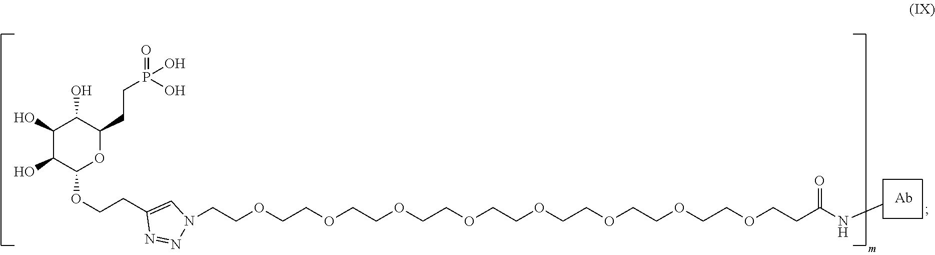

or a pharmaceutically acceptable salt thereof, wherein:

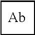

is an antibody or antibody fragment.

In certain embodiments, the bridging composition of formulas (I)-(Ib) is of formula (X):

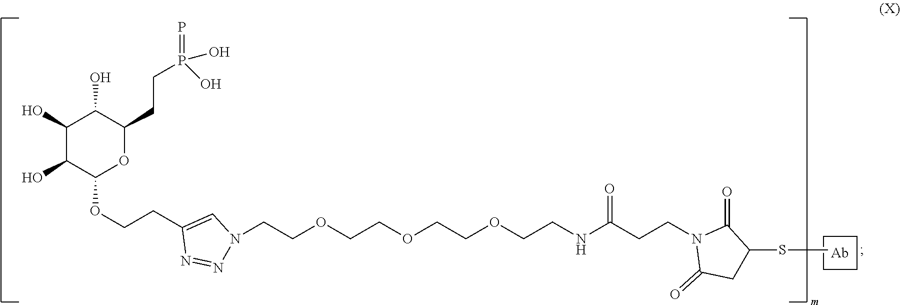

or a pharmaceutically acceptable salt thereof, wherein:

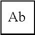

is an antibody or antibody fragment.

In certain embodiments, the bridging composition of formulas (I)-(Ib) is of formula (XI):

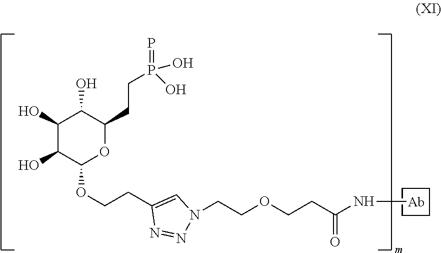

or a pharmaceutically acceptable salt thereof, wherein:

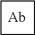

is an antibody or antibody fragment.

In certain embodiments, the bridging composition of formulas (I)-(Ib) is of formula (XII):

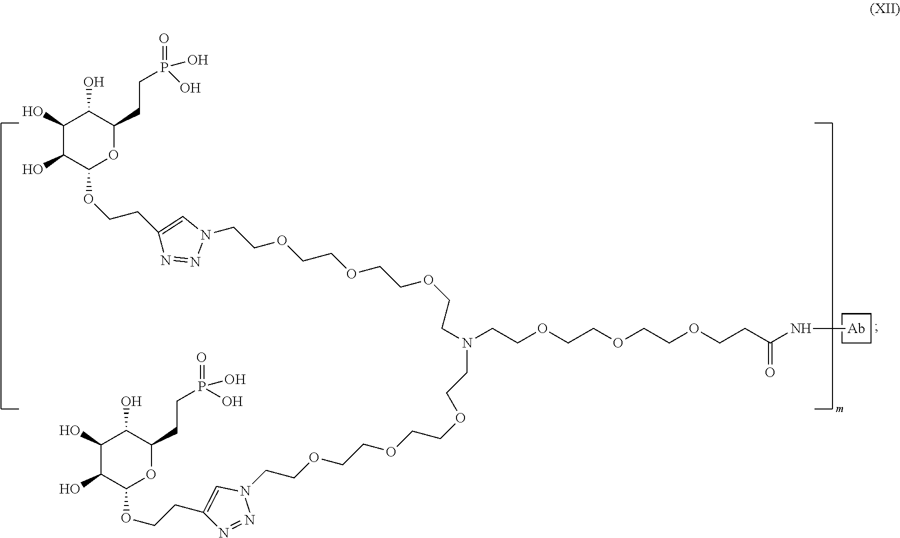

or a pharmaceutically acceptable salt thereof, wherein:

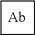

is an antibody or antibody fragment.

In some embodiments of the exemplary Ab containing bridging compositions described above, e.g., conjugates of formula (IX) to (XII), m is from 1 to 10. In some embodiments of the exemplary Ab containing bridging compositions described above, e.g., conjugates of formula (IX) to (XII), m is from 1 to 8. In some embodiments of the exemplary Ab containing bridging compositions described above, e.g., conjugates of formula (IX) to (XII), m is from 1 to 4. It is understood that depending on the conjugation chemistry used and site(s) of Ab conjugation, m can be referred to as an average loading per Ab.

5.2.6. Fusion Proteins

In certain embodiments, the modified viral composition may comprise a viral composition specifically bound to a bridging composition that comprises a bridging moiety and a cell surface binding moiety that are fused directly. For example, in particular embodiments, the bridging moiety comprises a protein and the cell surface receptor binding moiety comprises a protein, and the two proteins are directly fused to each other, e.g., directly or via a peptide linkage.

In certain embodiments, the bridging moiety and a cell surface binding moiety are fused indirectly. For example, in particular embodiments, the bridging moiety comprises a protein and the cell surface binding moiety comprises a protein, wherein the two proteins are indirectly fused to each other, e.g., via an intervening amino acid sequence and flanking peptide linkages.

The cell surface binding moiety (either with or without an intervening amino acid sequence at one or both ends) may be genetically encoded to be fused to the amino terminus of, the carboxy terminus of, or inserted within the bridging moiety as a heterologous peptide at a residue position that will permit the cell surface binding moiety to be exposed and continue to be capable of binding a cell surface receptor when present as part of a modified viral composition.

For example, routine techniques may be followed for engineering a coding sequence for the cell surface binding moiety protein amino acid to be placed, in-frame, into a coding sequence for the bridging moiety protein such that the cell surface binding moiety protein and the bridging moiety protein are expressed as a single fusion polypeptide wherein the cell surface binding moiety is inserted within the bridging moiety protein amino acid sequence at the desired position. Alternatively, a split-intein system may be utilized such that fusion polypeptide is formed post-translationally via protein splicing.

In particular embodiments, the modified viral composition comprises an AAV composition specifically bound to a bridging composition.

In certain embodiments, the N-terminus of the bridging moiety polypeptide is fused to the C-terminus of the cell surface binding moiety polypeptide directly, for example are directly fused via a peptide linkage, or indirectly fused via an intervening amino acid sequence.

In certain embodiments, the C-terminus of the bridging moiety polypeptide is fused to the N-terminus of the cell surface binding moiety polypeptide directly, or indirectly, for example are indirectly fused via an intervening amino acid sequence.

In certain embodiments, the modified viral composition is a modified AAV composition comprising a viral composition bound to a bridging composition, wherein the bridging composition comprises a polypeptide bridging moiety and a cell surface binding moiety wherein the N-terminus or the C-terminus of the bridging moiety polypeptide is attached.

In certain embodiments, the cell surface binding moiety polypeptide and the bridging moiety polypeptide are present as a single fusion polypeptide, and wherein the amino acid sequence of the cell surface binding moiety is present in the fusion polypeptide within the amino acid sequence of the bridging moiety polypeptide, wherein the cell surface binding moiety polypeptide sequence is optionally accompanied by an intervening amino acid sequence at the amino end, the carboxy end, or the amino and carboxy ends of the cell surface binding moiety sequence.

In certain embodiments, the bridging composition comprises a bridging moiety polypeptide and a cell surface binding moiety polypeptide and one or more linker sequences. Such linker sequences may, for example, comprise a linker sequence or sequences comprising glycine, serine and/or alanine amino acid residues, e.g., Gly-Gly-Gly-Gly-Ala (SEQ ID NO: 17) (for example 1-3 repeats of such a sequence) or Gly-Gly-Gly-Gly-Ser (SEQ ID NO: 18) (for example 1-3 repeats of such a sequence). A linker sequence may, for example, be present in the bridging composition between the binding moiety polypeptide and the cell surface binding moiety polypeptide (whether the bridging moiety is present amino to or carboxy to the cell surface binding moiety), or may be present amino to and/or carboxy to the cell surface binding moiety sequence (when the cell surface binding moiety polypeptide is present within the bridging moiety polypeptide sequence). An intervening linker sequence may also be present in instances when the cell surface binding moiety is attached, e.g., conjugated to fused to the binding moiety at a bridging moiety amino acid side chain, or alternatively when the bridging moiety polypeptide is attached (e.g., conjugated or fused) to the cell surface binding moiety polypeptide at a cell surface binding moiety polypeptide amino acid side chain.

Throughout, it is to be understood that reference to attachment of the bridging moiety to the cell surface binding moiety encompasses attachment when either or both of the moieties comprises more than one molecule, for example when the bridging moiety is an antibody or an antigen-binding fragment of an antibody, wherein the antibody or antigen-binding fragment comprise more than one molecule, e.g., comprises an antibody light chain and an antibody heavy chain, or two light chain-heavy chain pairs. In such instances, attachment may include attachment of any molecule of the bridging moiety to any molecule of the cell surface binding moiety. In such instances, reference to attachment to or of an N-terminus or C-terminus of one of the moieties to the other encompasses attachment to or of any (or all) such termini. Further, reference to insertion of one moiety within the sequence of the other encompasses insertion into any of the molecules of the moiety into which the sequence is being inserted. Likewise, in instances where a bridging moiety and/or a cell surface binding moiety comprise more than one polypeptide strand, reference to a bridging composition comprising a fusion polypeptide encompasses a bridging composition containing a fusion polypeptide comprising one strand of a bridging moiety and/or one strand of a cell surface binding moiety.

For example, in instances wherein a bridging moiety comprises a multi-strand (e.g., two or four polypeptide) antibody or antigen-binding fragment of an antibody, the cell surface binding moiety may be attached to any (or all) of the strands. Similarly, in a non-limiting example, in instances wherein a bridging moiety comprises a multi-strand (e.g., two or four polypeptide) antibody or antigen-binding fragment of an antibody, and a cell surface binding moiety comprises a polypeptide, the cell surface binding moiety may be attached to the N-terminus of any (or all) of the strands of the bridging moiety. In another non-limiting example, in instances wherein a bridging moiety comprises a multi-strand (e.g., two or four polypeptide) antibody or antigen-binding fragment of an antibody, and a cell surface binding moiety comprises a polypeptide, the cell surface moiety sequence may be inserted into any (or all) of the strands of the bridging moiety. In yet another non-limiting example, in instances wherein a bridging composition comprises a fusion protein and wherein the bridging moiety comprises a multi-strand (e.g., two or four polypeptide) antibody or antigen-binding fragment of an antibody, and the cell surface binding moiety comprises a polypeptide, a fusion protein may comprise the cell surface binding moiety and any of the strands of the bridging moiety.

In certain embodiments, the cell surface binding moiety amino acid sequence is attached to or inserted into the bridging moiety amino sequence along with a linker sequence, e.g., a linker sequence amino to the cell surface binding moiety amino acid sequence, a linker carboxy to the cell surface binding moiety amino acid sequence, or linker sequences amino to and carboxy to the cell surface binding moiety amino acid sequence. In a specific embodiment, the linker sequence may, for example, comprise a flanking linker sequence or sequences comprising glycine, serine and/or alanine amino acid residues, e.g., Gly-Gly-Gly-Gly-Ala (SEQ ID NO: 17) (for example 1-3 repeats of such a sequence) or Gly-Gly-Gly-Gly-Ser (SEQ ID NO: 18) (for example 1-3 repeats of such a sequence).

In certain embodiments, the cell surface binding moiety (either with or without an intervening amino acid sequence at one or both ends) may be genetically encoded to be fused to the amino terminus of, the carboxy terminus of, or inserted within bridging moiety polypeptide at a residue position that will permit the cell surface binding moiety to be exposed and continue to be capable of binding a cell surface receptor even when present as part of a modified viral composition as presented herein.

For example, routine techniques may be followed for engineering a coding sequence for the cell surface binding moiety protein amino acid to be placed, in-frame, into a coding sequence for the bridging moiety polypeptide such that the that the cell surface binding moiety protein and the bridging moiety polypeptide are expressed as a single fusion polypeptide wherein the cell surface binding moiety is inserted within the bridging moiety amino acid sequence at the desired position. Alternatively, a split-intein system may be utilized such that a fusion polypeptide is formed post-translationally via protein splicing.

In certain embodiments, the cell surface binding moiety of the modified viral composition comprises a polypeptide that binds to a cell surface receptor, for example, an M6PR, e.g., CI-M6PR, a folate receptor, e.g., a folate receptor 1 (FRα), or 2 (FRβ) receptor, or an asialoglycoprotein receptor.

In a specific embodiment, the cell surface binding moiety polypeptide comprises an insulin-like growth factor 2 (IGF2) amino acid sequence that binds an M6PR, e.g., a CI-M6PR. In another specific embodiment, the bridging moiety polypeptide comprises an antibody or antigen-binding fragment of an antibody, that specifically binds to a viral composition, for example, a virus particle, a virus capsid, or a viral protein, e.g., a viral capsid protein or envelope protein.

In certain embodiments, the cell surface receptor binding moiety that is fused to a bridging moiety is an IGF-2 polypeptide (e.g., as described herein). In some embodiments, the bridging moiety fused to an IGF-2 polypeptide is an antibody or antibody fragment. In some embodiments, the bridging moiety is an anti-AAV antibody or antibody fragment. In some embodiments, the bridging moiety is an anti-AAV8 antibody.

In some embodiments of formula (I), the bridging composition includes a polypeptide of the following formula (II):

V₁-L₁-X-L₂-V₂ (II)

wherein:

X is cell surface binding moiety heterologous to P (e.g., a IGF-2 polypeptide), that binds to cell surface receptor (e.g., CI-M6PR);

L₁ and L₂ are independently optional linkers which, if present are each attached to X via a peptide linkage;

V₁ is an amino-terminal amino acid sequence of P and V₂ is a carboxy-terminal portion of P, wherein P is a bridging moiety (e.g., an antibody or antibody fragment) that comprises, in an amino to carboxy direction, V₁ and V₂, and V₁ and V₂ are attached to X (or, if present, to L₁ and L₂, respectively).

In particular embodiments of formula (II), L₁ is present. In particular embodiments of formula (II), L₂ is present. In particular embodiments, X is a glycoprotein. In specific embodiments, X comprises an insulin-like growth factor 2 (IGF-2) polypeptide.

Exemplary fusion protein constructs for bifunctional bridging compositions and sequences are shown in Table 13 below. ADK8 is an exemplary anti-AAV8 antibody utilized in the examples of this disclosure that is incorporated into a fusion protein with IGF-2 polypeptide. A20 is an exemplary monoclonal anti-AAV2 antibody (3JIS in RCSB protein data bank (PDB)) utilized in the examples of this disclosure that is incorporated into a fusion protein with IGF-2 polypeptide. B1 is an exemplary monoclonal anti-AAV (VP1/VP2/VP3) antibody that recognizes multiple serotypes, utilized in the examples of this disclosure that is incorporated into a fusion protein with IGF-2 polypeptide. The IGF-2 polypeptide is fused to the N and C-terminus of the light and heavy chains.

TABLE 13
Exemplary IGF-2-Ab fusion bifunctional protein

| Ab | construct | Exemplary fusion protein sequence |
|---|---|---|
| ADK8 | Mouse | DVVMTQTPLTLSVTIGQPASISCKSSQSLLESDGKTYLNWLLQRPGQSP |
| | kappa | KRLIYLVSTLDSGVPDRFTGSGSGTDFTLKISRLEAEDLGVYYCWQGTH |
| | light | FPPTFGGGTKLEIKRADAAPTVSIFPPSSEQLTSGGASVVCFLNNFYPKDI |
| | chain | NVKWKIDGSERQNGVLNSWTDQDSKDSTYSMSSTLTLTKDEYERHNS |
| | | YTCEATHKTSTSPIVKSFNRNEC (SEQ ID NO: 19) |
| | Mouse | DVVMTQTPLTLSVTIGQPASISCKSSQSLLESDGKTYLNWLLQRPGQSP |
| | LC-IGF2 | KRLIYLVSTLDSGVPDRFTGSGSGTDFTLKISRLEAEDLGVYYCWQGTH |
| | fusion | FPPTFGGGTKLEIKRADAAPTVSIFPPSSEQLTSGGASVVCFLNNFYPKDI |
| | | NVKWKIDGSERQNGVLNSWTDQDSKDSTYSMSSTLTLTKDEYERHNS |
| | | YTCEATHKTSTSPIVKSFNRNECGGGGSLCGGELVDTLQFVCGDRGFYF |
| | | SRPASRVSRRSRGIVEECCFRSCDLALLETYCATPAKSE (SEQ ID NO: 20) |
| | IGF2- | LCGGELVDTLQFVCGDRGFYFSRPASRVSRRSRGIVEECCFRSCDLALL |
| | Mouse | ETYCATPAKSEGGGGSDVVMTQTPLTLSVTIGQPASISCKSSQSLLESDG |
| | LC | KTYLNWLLQRPGQSPKRLIYLVSTLDSGVPDRFTGSGSGTDFTLKISRLE |
| | fusion | AEDLGVYYCWQGTHFPPTFGGGTKLEIKRADAAPTVSIFPPSSEQLTSG |
| | | GASVVCFLNNFYPKDINVKWKIDGSERQNGVLNSWTDQDSKDSTYSM |
| | | SSTLTLTKDEYERHNSYTCEATHKTSTSPIVKSFNRNEC (SEQ ID NO: 21) |
| | Human | DVVMTQTPLTLSVTIGQPASISCKSSQSLLESDGKTYLNWLLQRPGQSP |
| | kappa | KRLIYLVSTLDSGVPDRFTGSGSGTDFTLKISRLEAEDLGVYYCWQGTH |
| | light | FPPTFGGGTKLEIKRTVAAPSVFIFPPSDEQLKSGTASVVCLLNNFYPRE |
| | chain | AKVQWKVDNALQSGNSQESVTEQDSKDSTYSLSSTLTLSKADYEKHK |
| | | VYACEVTHQGLSSPVTKSFNRGEC (SEQ ID NO: 22) |
| | Human | DVVMTQTPLTLSVTIGQPASISCKSSQSLLESDGKTYLNWLLQRPGQSP |
| | LC-IGF2 | KRLIYLVSTLDSGVPDRFTGSGSGTDFTLKISRLEAEDLGVYYCWQGTH |
| | fusion | FPPTFGGGTKLEIKRTVAAPSVFIFPPSDEQLKSGTASVVCLLNNFYPRE |
| | | AKVQWKVDNALQSGNSQESVTEQDSKDSTYSLSSTLTLSKADYEKHK |
| | | VYACEVTHQGLSSPVTKSFNRGECGGGGSLCGGELVDTLQFVCGDRGF |
| | | YFSRPASRVSRRSRGIVEECCFRSCDLALLETYCATPAKSE (SEQ ID NO: |
| | | 23) |
| | IGF2- | LCGGELVDTLQFVCGDRGFYFSRPASRVSRRSRGIVEECCFRSCDLALL |
| | Human | ETYCATPAKSEGGGGSDVVMTQTPLTLSVTIGQPASISCKSSQSLLESDG |
| | LC | KTYLNWLLQRPGQSPKRLIYLVSTLDSGVPDRFTGSGSGTDFTLKISRLE |
| | fusion | AEDLGVYYCWQGTHFPPTFGGGTKLEIKRTVAAPSVFIFPPSDEQLKSG |
| | | TASVVCLLNNFYPREAKVQWKVDNALQSGNSQESVTEQDSKDSTYSLS |
| | | STLTLSKADYEKHKVYACEVTHQGLSSPVTKSFNRGEC (SEQ ID NO: |
| | | 24) |
| | Mouse | DVKLVESGGGLVKPGGSLKLSCADSGFSFSSYSMSWVRQTPEKRLEWV |
| | IgG2a | ATITSGGDYTYYPDSVKGRFTISKDNARNTLYLQMSSLRSEDTAMYYCI |
| | heavy | RDFYGSTYWYFDVWGAGTTVTVSSAKTTAPSVYPLAPVCGDTTGSSVT |
| | chain | LGCLVKGYFPEPVTLTWNSGSLSSGVHTFPAVLQSDLYTLSSSVTVTSST |
| | | WPSQSITCNVAHPASSTKVDKKIEPRGPTIKPCPPCKCPAPNAAGGPSVFI |
| | | FPPKIKDVLMISLSPIVTCVVVDVSEDDPDVQISWFVNNVEVHTAQTQT |
| | | HREDYNSTLRVVSALPIQHQDWMSGKEFKCKVNNKDLGAPIERTISKP |
| | | KGSVRAPQVYVLPPPEEEMTKKQVTLTCMVTDFMPEDIYVEWTNNGK |
| | | TELNYKNTEPVLDSDGSYFMYSKLRVEKKNWVERNSYSCSVVHEGLH |
| | | NHHTTKSFSRTPGK (SEQ ID NO: 25) |
| | Human | DVKLVESGGGLVKPGGSLKLSCADSGFSFSSYSMSWVRQTPEKRLEWV |
| | IgG1 | ATITSGGDYTYYPDSVKGRFTISKDNARNTLYLQMSSLRSEDTAMYYCI |
| | heavy | RDFYGSTYWYFDVWGAGTTVTVSSASTKGPSVFPLAPSSKSTSGGTAA |
| | chain | LGCLVKDYFPEPVTVSWNSGALTSGVHTFPAVLQSSGLYSLSSVVTVPS |
| | | SSLGTQTYTCNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK (SEQ ID NO: 26) |
| | Mouse | DVKLVESGGGLVKPGGSLKLSCADSGFSFSSYSMSWVRQTPEKRLEWV |
| | HC-IGF2 | ATITSGGDYTYYPDSVKGRFTISKDNARNTLYLQMSSLRSEDTAMYYCI |
| | fusion | RDFYGSTYWYFDVWGAGTTVTVSSAKTTAPSVYPLAPVCGDTTGSSVT |
| | | LGCLVKGYFPEPVTLTWNSGSLSSGVHTFPAVLQSDLYTLSSSVTVTSST |
| | | WPSQSITCNVAHPASSTKVDKKIEPRGPTIKPCPPCKCPAPNAAGGPSVFI |
| | | FPPKIKDVLMISLSPIVTCVVVDVSEDDPDVQISWFVNNVEVHTAQTQT |
| | | HREDYNSTLRVVSALPIQHQDWMSGKEFKCKVNNKDLGAPIERTISKP |
| | | KGSVRAPQVYVLPPPEEEMTKKQVTLTCMVTDFMPEDIYVEWTNNGK |
| | | TELNYKNTEPVLDSDGSYFMYSKLRVEKKNWVERNSYSCSVVHEGLH |
| | | NHHTTKSFSRTPGKGGGGSLCGGELVDTLQFVCGDRGFYFSRPASRVSR |
| | | RSRGIVEECCFRSCDLALLETYCATPAKSE (SEQ ID NO: 27) |
| | IGF2- | LCGGELVDTLQFVCGDRGFYFSRPASRVSRRSRGIVEECCFRSCDLALL |
| | Mouse | ETYCATPAKSEGGGGSDVKLVESGGGLVKPGGSLKLSCADSGFSFSSYS |
| | HC | MSWVRQTPEKRLEWVATITSGGDYTYYPDSVKGRFTISKDNARNTLYL |
| | fusion | QMSSLRSEDTAMYYCTRDFYGSTYWYFDVWGAGTTVTVSSAKTTAPS |
| | | VYPLAPVCGDTTGSSVTLGCLVKGYFPEPVTLTWNSGSLSSGVHTFPAV |
| | | PPCKCPAPNAAGGPSVFIFPPKTKDVLMISLSPIVTCVVVDVSEDDPDVQI |
| | | SWFVNNVEVHTAQTQTHREDYNSTLRVVSALPIQHQDWMSGKEFKCK |
| | | VNNKDLGAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWV |
| | | ERNSYSCSVVHEGLHNHHTTKSFSRTPGK (SEQ ID NO: 28) |
| | Human | DVKLVESGGGLVKPGGSLKLSCADSGFSFSSYSMSWVRQTPEKRLEWV |
| | HC-IGF2 | ATITSGGDYTYYPDSVKGRFTISKDNARNTLYLQMSSLRSEDTAMYYCI |
| | fusion | RDFYGSTYWYFDVWGAGTTVTVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGKGGGGSLCGGELVDTLQFVCGDRGFYFSRPASRV |
| | | SRRSRGIVEECCFRSCDLALLETYCATPAKSE (SEQ ID NO: 29) |
| | IGF2- | LCGGELVDTLQFVCGDRGFYFSRPASRVSRRSRGIVEECCFRSCDLALL |
| | Human | ETYCATPAKSEGGGGSDVKLVESGGGLVKPGGSLKLSCADSGFSFSSYS |
| | HC | MSWVRQTPEKRLEWVATITSGGDYTYYPDSVKGRFTISKDNARNTLYL |
| | fusion | QMSSLRSEDTAMYYCIRDFYGSTYWYFDVWGAGTTVTVSSASTKGPS |
| | | VFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSCDK |
| | | THTCPPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSR |
| | | WQQGNVFSCSVMHEALHNHYTQKSLSLSPGK (SEQ ID NO: 30) |
| A20 | Mouse | DIQMTQSSSSFSVSLGDRVTITCKASEDIHNRLAWYKQKPGNAPRLLISG |
| | kappa | ATSLETGVPSRFSGSGSGKDYTLSITSLQNEDVATYYCQQYWIGPFTFGS |
| | light | GTNLEIKRADAAPTVSIFPPSSEQLTSGGASVVCFLNNFYPKDINVKWKI |
| | chain | DGSERQNGVLNSWTDQDSKDSTYSMSSTLTLTKDEYERHNSYTCEATH |
| | | KTSTSPIVKSFNRNEC (SEQ ID NO: 31) |
| | Mouse | DIQMTQSSSSFSVSLGDRVTITCKASEDIHNRLAWYKQKPGNAPRLLISG |
| | LC-IGF2 | ATSLETGVPSRFSGSGSGKDYTLSITSLQNEDVATYYCQQYWIGPFTFGS |
| | fusion | GTNLEIKRADAAPTVSIFPPSSEQLTSGGASVVCFLNNFYPKDINVKWKI |
| | | DGSERQNGVLNSWTDQDSKDSTYSMSSTLTLTKDEYERHNSYTCEATH |
| | | KTSTSPIVKSFNRNECGGGGSLCGGELVDTLQFVCGDRGFYFSRPASRV |
| | | SRRSRGIVEECCFRSCDLALLETYCATPAKSE (SEQ ID NO: 32) |
| | IGF2- | LCGGELVDTLQFVCGDRGFYFSRPASRVSRRSRGIVEECCFRSCDLALL |
| | Mouse | ETYCATPAKSEGGGGSDIQMTQSSSSFSVSLGDRVTITCKASEDIHNRLA |
| | LC | WYKQKPGNAPRLLISGATSLETGVPSRFSGSGSGKDYTLSITSLQNEDV |
| | fusion | ATYYCQQYWIGPFTFGSGTNLEIKRADAAPTVSIFPPSSEQLTSGGASVV |
| | | CFLNNFYPKDINVKWKIDGSERQNGVLNSWTDQDSKDSTYSMSSTLTL |
| | | TKDEYERHNSYTCEATHKTSTSPIVKSFNRNEC (SEQ ID NO: 33) |
| | Human | DIQMTQSSSSFSVSLGDRVTITCKASEDIHNRLAWYKQKPGNAPRLLISG |
| | kappa | ATSLETGVPSRFSGSGSGKDYTLSITSLQNEDVATYYCQQYWIGPFTFGS |
| | light | GTNLEIKRTVAAPSVFIFPPSDEQLKSGTASVVCLLNNFYPREAKVQWK |
| | chain | VDNALQSGNSQESVTEQDSKDSTYSLSSTLTLSKADYEKHKVYACEVT |
| | | HQGLSSPVTKSFNRGEC (SEQ ID NO: 34) |
| | Human | DIQMTQSSSSFSVSLGDRVTITCKASEDIHNRLAWYKQKPGNAPRLLISG |
| | LC-IGF2 | ATSLETGVPSRFSGSGSGKDYTLSITSLQNEDVATYYCQQYWIGPFTFGS |
| | fusion | GTNLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGECGGGGSLCGGELVDTLQFVCGDRGFYFSRPASR |
| | | VSRRSRGIVEECCFRSCDLALLETYCATPAKSE (SEQ ID NO: 35) |
| | IGF2- | LCGGELVDTLQFVCGDRGFYFSRPASRVSRRSRGIVEECCFRSCDLALL |
| | Human | ETYCATPAKSEGGGGSDIQMTQSSSSFSVSLGDRVTTTCKASEDIHNRLA |
| | LC | WYKQKPGNAPRLLISGATSLETGVPSRFSGSGSGKDYTLSITSLQNEDV |
| | fusion | ATYYCQQYWIGPFTFGSGTNLEIKRTVAAPSVFIFPPSDEQLKSGTASVV |
| | | CLLNNFYPREAKVQWKVDNALQSGNSQESVTEQDSKDSTYSLSSTLTL |
| | | SKADYEKHKVYACEVTHQGLSSPVTKSFNRGEC (SEQ ID NO: 36) |
| | Mouse | SDVQLQESGPDLVKPSQSLSLTCTVTGYSITSGYTWHWIRQFPGNKQEW |
| | IgG2a | MGYIHFSGYTNYNPSLKSRVSITRDTSKNQFFLHLNSVTTEDTATYYCA |
| | heavy | RGDYGYEWFTYWGQGTLVTVSAAKTTAPSVYPLAPVCGDTTGSSVTL |
| | chain | GCLVKGYFPEPVTLTWNSGSLSSGVHTFPAVLQSDLYTLSSSVTVTSST |
| | | WPSQSITCNVAHPASSTKVDKKIEPRGPTTKPCPPCKCPAPNAAGGPSVFI |
| | | FPPKIKDVLMISLSPIVTCVVVDVSEDDPDVQISWFVNNVEVHTAQTQT |
| | | HREDYNSTLRVVSALPIQHQDWMSGKEFKCKVNNKDLGAPIERTISKP |
| | | KGSVRAPQVYVLPPPEEEMTKKQVTLTCMVTDFMPEDIYVEWTNNGK |
| | | TELNYKNTEPVLDSDGSYFMYSKLRVEKKNWVERNSYSCSVVHEGLH |
| | | NHHTTKSFSRTPGK (SEQ ID NO: 37) |
| | Human | SDVQLQESGPDLVKPSQSLSLTCTVTGYSITSGYTWHWIRQFPGNKQEW |
| | IgG1 | MGYIHFSGYTNYNPSLKSRVSITRDTSKNQFFLHLNSVTIEDTATYYCA |
| | heavy | RGDYGYEWFTYWGQGTLVTVSAASTKGPSVFPLAPSSKSTSGGTAALG |
| | chain | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPSREEMTKNQVSLTCLVKGFYPSDTAVEWESNG |
| | | QPENNYKTTPPVLDSDGSFFLYSKLTVDKSRWQQGNVFSCSVMHEALH |
| | | NHYTQKSLSLSPGK (SEQ ID NO: 38) |
| | Mouse | SDVQLQESGPDLVKPSQSLSLTCTVTGYSTTSGYTWHWIRQFPGNKQEW |
| | HC-IGF2 | MGYIHFSGYTNYNPSLKSRVSITRDTSKNQFFLHLNSVTTEDTATYYCA |
| | fusion | RGDYGYEWFTYWGQGTLVTVSAAKTTAPSVYPLAPVCGDTTGSSVTL |
| | | GCLVKGYFPEPVTLTWNSGSLSSGVHTFPAVLQSDLYTLSSSVTVTSST |
| | | WPSQSITCNVAHPASSTKVDKKTEPRGPTIKPCPPCKCPAPNAAGGPSVFI |
| | | FPPKIKDVLMISLSPIVTCVVVDVSEDDPDVQISWFVNNVEVHTAQTQT |
| | | HREDYNSTLRWSALPIQHQDWMSGKEFKCKXWIKDLGAPIERTISKP |
| | | KGSVRAPQVYVLPPPEEEMTKKQVTLTCMVTDFMPEDIYVEWTNNGK |
| | | TELNYKNTEPVLDSDGSYFMYSKLRVEKKNWVERNSYSCSVVHEGLH |
| | | NHHTTKSFSRTPGKGGGGSLCGGELVDTLQFVCGDRGFYFSRPASRVSR |
| | | RSRGIVEECCFRSCDLALLETYCATPAKSE (SEQ ID NO: 39) |
| | IGF2- | LCGGELVDTLQFVCGDRGFYFSRPASRVSRRSRGIVEECCFRSCDLALL |
| | Mouse | ETYCATPAKSEGGGGSSDVQLQESGPDLVKPSQSLSLTCTVTGYSITSG |
| | HC | YTWHWIRQFPGNKQEWMGYIHFSGYTNYNPSLKSRVSITRDTSKNQFF |
| | fusion | LHLNSVTTEDTATYYCARGDYGYEWFTYWGQGTLVTVSAAKTTAPSV |
| | | YPLAPVCGDTTGSSVTLGCLVKGYFPEPVTLTWNSGSLSSGVHTFPAVL |
| | | QSDLYTLSSSVTVTSSTWPSQSITCNVAHPASSTKVDKKIEPRGPTIKPCP |
| | | PCKCPAPNAAGGPSVFIFPPKIKDVLMISLSPIVTCVVVDVSEDDPDVQIS |
| | | WFVNNVEVHTAQTQTHREDYNSTLRVVSALPIQHQDWMSGKEFKCKV |
| | | NNKDLGAPIERTISKPKGSVRAPQVYVLPPPEEEMTKKQVTLTCMVTDF |
| | | MPEDIYVEWTNNGKTELNYKNTEPVLDSDGSYFMYSKLRVEKKNWVE |
| | | RNSYSCSVVHEGLHNHHTTKSFSRTPGK (SEQ ID NO: 40) |
| | Human | SDVQLQESGPDLVKPSQSLSLTCTVTGYSITSGYTWHWIRQFPGNKQEW |
| | HC-IGF2 | MGYIHFSGYTNYNPSLKSRVSITRDTSKNQFFLHLNSVTTEDTATYYCA |
| | fusion | RGDYGYEWFTYWGQGTLVTVSAASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGKGGGGSLCGGELVDTLQFVCGDRGFYFSRPASRVS |
| | | RRSRGIVEECCFRSCDLALLETYCATPAKSE (SEQ ID NO: 41) |
| | IGF2- | LCGGELVDTLQFVCGDRGFYFSRPASRVSRRSRGIVEECCFRSCDLALL |
| | Human | ETYCATPAKSEGGGGSSDVQLQESGPDLVKPSQSLSLTCTVTGYSITSG |
| | HC | YTWHWIRQFPGNKQEWMGYIHFSGYTNYNPSLKSRVSITRDTSKNQFF |
| | fusion | LHLNSVTTEDTATYYCARGDYGYEWFTYWGQGTLVTVSAASTKGPSV |
| | | FPLAPSSKSTSGGTAALGCLVKDYFPEPVTVSWNSGALTSGVHTFPAVL |
| | | QSSGLYSLSSVVTVPSSSLGTQTYTCNVNHKPSNTKVDKKVEPKSCDKT |
| | | HTCPPCPAPEAAGGPSVFLFPPKPKDTLMISRTPEVTCVVVDVSHEDPEV |
| | | KFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSR |
| | | WQQGNVFSCSVMHEALHNHYTQKSLSLSPGK (SEQ ID NO: 42) |
| B1 | Mouse | DIVMSQSPSSLTVSVGEKVTMSCKSSQSLLYSTNQKNYLAWYQQKPGQ |
| | kappa | SPKLLIFWASTRESGVPDRFTGSGSGTDFTLTISSVKAEDLAVYYCQQY |
| | light | YRYLTFGTGTKLELRRADAAPTVSIFPPSSEQLTSGGASVVCFLNNFYPK |
| | chain | DINVKWKIDGSERQNGVLNSWTDQDSKDSTYSMSSTLTLTKDEYERHN |
| | | SYTCEATHKTSTSPIVKSFNRNEC (SEQ ID NO: 43) |
| | Mouse | DIVMSQSPSSLTVSVGEKVTMSCKSSQSLLYSTNQKNYLAWYQQKPGQ |
| | LC-IGF2 | SPKLLIFWASTRESGVPDRFTGSGSGTDFTLTISSVKAEDLAVYYCQQY |
| | fusion | YRYLTFGTGTKLELRRADAAPTVSIFPPSSEQLTSGGASVVCFLNNFYPK |
| | | DINVKWKIDGSERQNGVLNSWTDQDSKDSTYSMSSTLTLTKDEYERHN |
| | | SYTCEATHKTSTSPIVKSFNRNECGGGGSLCGGELVDTLQFVCGDRGFY |
| | | FSRPASRVSRRSRGIVEECCFRSCDLALLETYCATPAKSE (SEQ ID NO: |
| | | 44) |
| | IGF2- | LCGGELVDTLQFVCGDRGFYFSRPASRVSRRSRGIVEECCFRSCDLALL |
| | Mouse | ETYCATPAKSEGGGGSDIVMSQSPSSLTVSVGEKVTMSCKSSQSLLYST |
| | LC | NQKNYLAWYQQKPGQSPKLLIFWASTRESGVPDRFTGSGSGTDFTLTIS |
| | fusion | SVKAEDLAVYYCQQYYRYLTFGTGTKLELRRADAAPTVSIFPPSSEQLT |
| | | SGGASVVCFLNNFYPKDINVKWKIDGSERQNGVLNSWTDQDSKDSTYS |
| | | MSSTLTLTKDEYERHNSYTCEATHKTSTSPIVKSFNRNEC (SEQ ID NO: ) |
| | Human | DIVMSQSPSSLTVSVGEKVTMSCKSSQSLLYSTNQKNYLAWYQQKPGQ |
| | kappa | SPKLLIFWASTRESGVPDRFTGSGSGTDFTLTISSVKAEDLAVYYCQQY |
| | light | YRYLTFGTGTKLELRRTVAAPSVFIFPPSDEQLKSGTASVVCLLNNFYPR |
| | chain | EAKVQWKVDNALQSGNSQESVTEQDSKDSTYSLSSTLTLSKADYEKHK |
| | | VYACEVTHQGLSSPVTKSFNRGEC (SEQ ID NO: 45) |
| | Human | DIVMSQSPSSLTVSVGEKVTMSCKSSQSLLYSTNQKNYLAWYQQKPGQ |
| | LC-IGF2 | SPKLLIFWASTRESGVPDRFTGSGSGTDFTLTISSVKAEDLAVYYCQQY |
| | fusion | YRYLTFGTGTKLELRRTVAAPSVFIFPPSDEQLKSGTASVVCLLNNFYPR |
| | | EAKVQWKVDNALQSGNSQESVTEQDSKDSTYSLSSTLTLSKADYEKHK |
| | | VYACEVTHQGLSSPVTKSFNRGECGGGGSLCGGELVDTLQFVCGDRGF |
| | | YFSRPASRVSRRSRGIVEECCFRSCDLALLETYCATPAKSE (SEQ ID NO: |
| | | 46) |
| | IGF2- | LCGGELVDTLQFVCGDRGFYFSRPASRVSRRSRGIVEECCFRSCDLALL |
| | Human | ETYCATPAKSEGGGGSDIVMSQSPSSLTVSVGEKVTMSCKSSQSLLYST |
| | LC | NQKNYLAWYQQKPGQSPKLLIFWASTRESGVPDRFTGSGSGTDFTLTIS |
| | fusion | SVKAEDLAVYYCQQYYRYLTFGTGTKLELRRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 47) |
| | Mouse | QVQLQQSAAELVRPGASVTLSCKASGYTFNDHEMHWVKQTPVYGLE |
| | IgG2a | WIGAIDPETGGTAYNQKFKGKATLTADKSSSTAYMELRSLTSEDSAVY |
| | heavy | YCTGEGYWGQGTSVTVSSAKTTAPSVYPLAPVCGDTTGSSVTLGCLVK |
| | chain | GYFPEPVTLTWNSGSLSSGVHTFPAVLQSDLYTLSSSVTVTSSTWPSQSI |
| | | TCNVAHPASSTKVDKKIEPRGPTIKPCPPCKCPAPNAAGGPSVFIFPPKIK |
| | | DVLMISLSPIVTCVVVDVSEDDPDVQISWFVNNVEVHTAQTQTHREDY |
| | | NSTLRVVSALPIQHQDWMSGKEFKCKVNNKDLGAPIERTISKPKGSVRA |
| | | PQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTK |
| | | SFSRTPGK (SEQ ID NO: 48) |
| | Human | QVQLQQSAAELVRPGASVTLSCKASGYTFNDHEMHWVKQTPVYGLE |
| | IgG1 | WIGAIDPETGGTAYNQKFKGKATLTADKSSSTAYMELRSLTSEDSAVY |
| | heavy | YCTGEGYWGQGTSVTVSSASTKGPSVFPLAPSSKSTSGGTAALGCLVK |
| | chain | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYT |
| | | QKSLSLSPGK (SEQ ID NO: 49) |
| | Mouse | QVQLQQSAAELVRPGASVTLSCKASGYTFNDHEMHWVKQTPVYGLE |
| | HC-IGF2 | WIGAIDPETGGTAYNQKFKGKATLTADKSSSTAYMELRSLTSEDSAVY |
| | fusion | YCTGEGYWGQGTSVTVSSAKTTAPSVYPLAPVCGDTTGSSVTLGCLVK |
| | | GYFPEPVTLTWNSGSLSSGVHTFPAVLQSDLYTLSSSVTVTSSTWPSQSI |
| | | TCNVAHPASSTKVDKKIEPRGPTIKPCPPCKCPAPNAAGGPSVFIFPPKIK |
| | | DVLMISLSPIVTCVVVDVSEDDPDVQISWFVNNVEVHTAQTQTHREDY |
| | | NSTLRVVSALPIQHQDWMSGKEFKCKVNNKDLGAPIERTISKPKGSVRA |
| | | PQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTK |
| | | SFSRTPGKGGGGSLCGGELVDTLQFVCGDRGFYFSRPASRVSRRSRGIV |
| | | EECCFRSCDLALLETYCATPAKSE (SEQ ID NO: 50) |
| | IGF2- | LCGGELVDTLQFVCGDRGFYFSRPASRVSRRSRGIVEECCFRSCDLALL |
| | Mouse | ETYCATPAKSEGGGGSQVQLQQSAAELVRPGASVTLSCKASGYTFNDH |
| | HC | EMHWVKQTPVYGLEWIGAIDPETGGTAYNQKFKGKATLTADKSSSTA |
| | fusion | YMELRSLTSEDSAVYYCTGEGYWGQGTSVTVSSAKTTAPSVYPLAPVC |
| | | GDTTGSSVTLGCLVKGYFPEPVTLTWNSGSLSSGVHTFPAVLQSDLYTL |
| | | SSSVTVTSSTWPSQSITCNVAHPASSTKVDKKIEPRGPTIKPCPPCKCPAP |
| | | NAAGGPSVFIFPPKIKDVLMISLSPrVTCVVVDVSEDDPDVQISWFVNNV |
| | | EVHTAQTQTHREDYNSTLRVVSALPIQHQDWMSGKEFKCKVNNKDLG |
| | | APIERTISKPKGSVRAPQVYVLPPPEEEMTKKQVTLTCMVTDFMPEDIY |
| | | VEWTNNGKTELNYKNTEPVLDSDGSYFMYSKLRVEKKNWVERNSYSC |
| | | SVVHEGLHNHHTTKSFSRTPGK (SEQ ID NO: 51) |
| | Human | QVQLQQSAAELVRPGASVTLSCKASGYTFNDHEMHWVKQTPVYGLE |
| | HC-IGF2 | WIGAIDPETGGTAYNQKFKGKATLTADKSSSTAYMELRSLTSEDSAVY |
| | fusion | YCTGEGYWGQGTSVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYT |
| | | QKSLSLSPGKGGGGSLCGGELVDTLQFVCGDRGFYFSRPASRVSRRSRG |
| | | IVEECCFRSCDLALLETYCATPAKSE (SEQ ID NO: 52) |
| | IGF2- | LCGGELVDTLQFVCGDRGFYFSRPASRVSRRSRGIVEECCFRSCDLALL |
| | Human | ETYCATPAKSEGGGGSQVQLQQSAAELVRPGASVTLSCKASGYTFNDH |
| | HC | EMHWVKQTPVYGLEWIGAIDPETGGTAYNQKFKGKATLTADKSSSTA |
| | fusion | YMELRSLTSEDSAVYYCTGEGYWGQGTSVTVSSASTKGPSVFPLAPSSK |
| | | STSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPA |
| | | PEAAGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK(SEQ ID NO: 53) |

### 5.3. Modified Viral Compositions

As summarized above, aspects of this disclosure include modified viral compositions that comprise a viral composition and a separate bridging composition capable of binding the viral composition, or a component thereof. The bridging composition can further bind to an endocytic cell surface receptor (via binding of a binding moiety or ligand for a endocytic cell surface receptor) that mediates internalization of the modified viral composition. In certain embodiments, the modified viral composition includes a viral composition wherein the viral composition includes a viral protein, e.g., a viral capsid protein or viral envelope protein, bound to a bridging composition. In some embodiments, the viral protein is part of a virus particle or is capable of being assembled into a virus particle.

A viral composition can include, for example, a virus particle, a virus capsid or a viral protein (e.g., a viral capsid protein or an envelope protein). In certain embodiments, a modified viral composition comprises a virus particle that comprises a polynucleotide that optionally comprises a transgene.

The terms “virus particle,” “viral particle,” “virus vector” or “viral vector” are used interchangeably herein. A “virus particle” refers to a virus capsid and a polynucleotide (DNA or RNA), which may comprise a viral genome, a portion of a viral genome, or a polynucleotide derived from a viral genome (e.g., one or more ITRs), which polynucleotide optionally comprises a transgene. In certain instances, a virus particle further comprises an envelope (which generally comprises lipid moieties and envelope proteins), surrounding or partially surrounding the capsid.

A viral particle may be referred to as a “recombinant viral particle,” or “recombinant virus particle,” which terms as used herein refer to a virus particle that has been genetically altered, e.g., by the deletion or other mutation of an endogenous viral gene and/or the addition or insertion of a heterologous nucleic acid construct into the polynucleotide of the virus particle. Thus, a recombinant virus particle generally refers to a virus particle comprising a capsid coat or shell (and an optional outer envelope) within which is packaged a polynucleotide sequence that comprises sequences of viral origin and sequences not of viral origin (i.e., a polynucleotide heterologous to the virus). This polynucleotide sequence is typically a sequence of interest for the genetic alteration of a cell.

In certain aspects of this disclosure, a viral composition described herein may comprise an “viral capsid,” “empty viral particle,” “empty virus particle,” or “capsid,” or “empty particle” when referred to herein in the context of the virus, which terms as used herein refer to a three-dimensional shell or coat comprising a viral capsid protein, optionally surrounded or partially surrounded by an outer envelope. In particular embodiments, the viral composition is a virus particle or a fragment thereof, virus capsid or fragment thereof, a viral protein, for example, a virus capsid protein or fragment thereof or envelope protein, or fragment thereof, of a virus of Table 14, or is derived from a virus of Table 14.

Any viral vector of interest, for example one capable of being applied for a therapeutic use, e.g., in a gene therapy, or manufacturing use, can be used in the present disclosure, for example, vectors derived from adenovirus (AV); adeno-associated virus (AAV); retroviruses (e.g., lentiviruses (LV), rhabdoviruses, murine leukemia virus); herpes simplex virus, and the like. Non-limiting examples are listed in Table 14 below. One of skill in the art will be able to routinely determine which properties of a virus are advantageous for a particular application and choose a suitable virus.

In certain embodiments, a virus used in a modified viral composition disclosed herein is a virus of the Duplodnaviria realm. In certain embodiments, a virus used in a modified viral composition disclosed herein is a virus of the Monodnaviria realm. In certain embodiments, a virus used in a modified viral composition disclosed herein is a virus of the Riboviria realm. In certain embodiments, a virus used in a modified viral composition disclosed herein is a virus of the Varidnaviria realm.

In certain embodiments, a virus used in a modified viral composition disclosed herein is a virus of the Bamfordvirae kingdom. In certain embodiments, a virus used in a modified viral composition disclosed herein is a virus of the Helvetiaviraa kingdom. In certain embodiments, a virus used in a modified viral composition disclosed herein is a virus of the Heunggongvirae kingdom. In certain embodiments, a virus used in a modified viral composition disclosed herein is a virus of the Loebvirae kingdom. In certain embodiments, a virus used in a modified viral composition disclosed herein is a virus of the Orthornavirae kingdom. In certain embodiments, a virus used in a modified viral composition disclosed herein is a virus of the Pararnavirae kingdom. In certain embodiments, a virus used in a modified viral composition disclosed herein is a virus of the Sangervirae kingdom. In certain embodiments, a virus used in a modified viral composition disclosed herein is a virus of the Shotokuvirae kingdom.

In certain embodiments, a virus used in a modified viral composition disclosed herein is a virus of the Artverviricota phylum. In certain embodiments, a virus used in a modified viral composition disclosed herein is a virus of the Cossaviricota phylum. In certain embodiments, a virus used in a modified viral composition disclosed herein is a virus of the Cressdnaviricota phylum. In certain embodiments, a virus used in a modified viral composition disclosed herein is a virus of the Dividoviricota phylum. In certain embodiments, a virus used in a modified viral composition disclosed herein is a virus of the Duplornaviricota phylum. In certain embodiments, a virus used in a modified viral composition disclosed herein is a virus of the Hofneiviricota phylum. In certain embodiments, a virus used in a modified viral composition disclosed herein is a virus of the Kitrinoviricota phylum. In certain embodiments, a virus used in a modified viral composition disclosed herein is a virus of the Lenarviricotaphylum. In certain embodiments, a virus used in a modified viral composition disclosed herein is a virus of the Negarnaviricota phylum. In certain embodiments, a virus used in a modified viral composition disclosed herein is a virus of the Nucleocytoviricota phylum. In certain embodiments, a virus used in a modified viral composition disclosed herein is a virus of the Peploviricota phylum. In certain embodiments, a virus used in a modified viral composition disclosed herein is a virus of the Phixviricota phylum. In certain embodiments, a virus used in a modified viral composition disclosed herein is a virus of the Pisuviricota phylum. In certain embodiments, a virus used in a modified viral composition disclosed herein is a virus of the Preplasmiviricota.

In certain embodiments, the virus used in a modified viral composition provided herein is a part of a virus family set forth in Table 14 below. In specific embodiments, the virus used in a modified viral composition provided herein falls within one of the non-limiting exemplary genera of viruses listed in Table 14 below.

For example, in some embodiments, the virus used in a modified viral composition provided herein is a Hepadnavirus, e.g., a Hepatitis B virus. In other embodiments, the virus used in a modified viral composition provided herein is a retrovirus, e.g., a lentivirus, for example, Human immunodeficiency virus 1 or Human immunodeficiency virus 2. In other specific embodiments, the virus used in a modified viral composition provided herein is papillomavirus, a human Alphapapillomavirus. In other specific embodiments, the virus used in a modified viral composition provided herein is a polyoma virus, for example, Macaca mulatta polyomavirus 1 (also called SV40). In other specific embodiments, the virus used in a modified viral composition provided herein is a togavirus, for example, Semliki Forest virus. In other specific embodiments, the virus used in a modified viral composition provided herein is an orthomyxovirus, for example, Influenza A virus, Influenza B virus, or Influenza D virus. In other specific embodiments, the virus used in a modified viral composition provided herein is a paramyxovirus, for example, Measles morbillivirus, or Avian orthoavulavirus 1 (e.g., Newcastle Disease virus). In other specific embodiments, the virus used in a modified viral composition provided herein is a rhabdovirus, for example. Indiana vesiculovirus (also called vesicular stomatitis virus) or Maraba vesiculovirus. In other specific embodiments, the virus used in a modified viral composition provided herein is a poxvirus, for example, vaccinia virus. In other specific embodiments, the virus used in a modified viral composition provided herein is an alloherpesvirus, for example, Human alphaherpesvirus 1 or Human alphaherpesvirus 2. In other specific embodiments, the virus used in a modified viral composition provided herein is a picornavirus, for example, an Enterovirus (e.g., Coxsackievirus, Echovirus or poliovirus). In other specific embodiments, the virus used in a modified viral composition provided herein is a herpesvirus, for example, Cytomegalovirus.

In other specific embodiments, the virus used in a modified viral composition provided herein is a coronavirus. In other specific embodiments, the virus used in a modified viral composition provided herein is an adenovirus. In other specific embodiments, the virus used in a modified viral composition provided herein is a reovirus.

In certain embodiments, a virus used in a modified viral composition provided herein is a human virus. In other embodiments, a virus used in a modified viral composition provided herein is an avian virus. In other embodiments, a virus used in a modified viral composition provided herein is a primate virus. In other embodiments, a virus used in a modified viral composition provided herein is an insect virus (e.g., Baculovirus).

In some embodiments, the virus used in a modified viral composition provided herein is derived from a virus listed in Table 14 below, or is derived from a virus of one of the exemplary genera listed in Table 14 below. For example, a virus used in a modified viral composition provided herein may not able to replicate in a host, e.g., a human host in the absence of a helper virus, e.g., may be engineered to not be able to do so.

A virus used in a modified viral composition provided herein may be a DNA virus or an RNA virus. For example, in some embodiments, a virus used in a modified viral composition provided herein is a double-stranded DNA virus. In other embodiments, a virus used in a modified viral composition provided herein is a single-stranded DNA virus. In other embodiments, a virus used in a modified viral composition provided herein is a single-stranded RNA virus, for example, a positive strand single-stranded RNA virus or a negative strand single-stranded RNA virus. In other embodiments, a virus used in a modified viral composition provided herein is a double-stranded RNA virus.

TABLE 14
Exemplary viruses.

| Realm | Kingdom | Phylum | Class | Order | Family |
|---|---|---|---|---|---|
| Duplodnaviria | Heunggongvirae | Peploviricota | Herviviricetes | Herpesvirales | Alloherpesviridae |
| | | | | | Herpesviridae |
| | | | | | Malacoherpesviridae |
| Monodnaviria | Loebvirae | Hofneiviricota | Faserviricetes | Tubulavirales | Inoviridae |
| | Sangervirae | Phixviricota | Malgrandaviricetes | Petitvirales | Microviridae |
| | Shotokuvirae | Cossaviricota | Mouviricetes | Polivirales | Bidnaviridae |
| | | | Papovaviricetes | Sepolyvirales | Polyomaviridae |
| | | | | Zurhausenvirales | Papillomaviridae |
| | | | Quintoviricetes | Piccovirales | Parvoviridae |
| | | Cre [symbol] | Arfiviricetes | Cirlivirales | Circoviridae |
| | | | | Cremevirales | Smacoviridae |
| | | | | Recrevirales | Redondoviridae |
| | | | Repensiviricetes | Geplafuvirales | Geminiviridae |
| | | | | | Genomoviridae |
| Riboviria | Orhornavirae | Duplornaviricota | Chrymotiviricetes | Ghabrivirales | Megabirnaviridae |
| | | | | | Quadriviridae |
| | | | Resentoviricetes | Reovirales | Reoviridae |
| | | Kitrinovincota | Alsuviricetes | Hepelivirales | Atphatetraviridae |
| | | | | | Hepeviridae |
| | | | | | Matonaviridae |
| | | | | Martellivirales | Mayoviridae |
| | | | | | Togaviridae |
| | | | | | Virgaviridae |
| | | | | Tymovirales | Alphaflexiviridae |
| | | | | | Tymoviridae |
| | | | Flasuviricetes | Amarillovirales | Flaviviridae |
| | | | Magsaviricetes | Nodamuvirales | Nodaviridae |
| | | | | | Sinhaliviridae |
| | | | Tolucaviricetes | Tolivirales | Carmotetraviridae |
| | | | | | Luteoviridae |
| | | | | | Tombusviridae |
| | | Lenarviricota | Miaviricetes | Ourlivirales | Botourmiaviridae |
| | | Negarnaviricota | Chunqiuviricetes | Muvirales | Qinviridae |
| | | | Ellioviricetes | Bunyavirales | Arenaviridae |
| | | | | | Cruliviridae |
| | | | | | Hantaviridae |
| | | | | | Leishbuviridae |
| | | | | | Mypoviridae |
| | | | | | Nairoviridae |
| | | | | | Peribunyaviridae |
| | | | | | Phasmaviridae |
| | | | | | Phenuiviridae |
| | | | | | Wupedeviridae |
| | | | Insthoviricetes | Articulavirales | Amnoonviridae |
| | | | | | Orthomyxoviridae |
| | | | Monjiviricetes | Jingchuvirales | Chuviridae |
| | | | | Mononegavirales | Artoviridae |
| | | | | | Bornaviridae |
| | | | | | Filoviridae |
| | | | | | Lispiviridae |
| | | | | | Nyamiviridae |
| | | | | | Paramyxoviridae |
| | | | | | Pneumoviridae |
| | | | | | Rhabdoviridae |
| | | | | | Sunviridae |
| | | | | | Xinmoviridae |
| | | | Yunchangviricetes | Goujianvirales | Yueviridae |
| | | Pisuviricota | Duplopiviricetes | Durnavirales | Amalgaviridae |
| | | | | | Picobirnaviridae |
| | | | Pisoniviricetes | Nidovirales | Abyssoviridae |
| | | | | | Arteriviridae |
| | | | | | Coronaviridae |
| | | | | | Cremegaviridae |
| | | | | | Euroniviridae |
| | | | | | Gresnaviridae |
| | | | | | Medioniviridae |
| | | | | | Mesoniviridae |
| | | | | | Mononiviridae |
| | | | | | Nanghoshaviridae |
| | | | | | Nanhypoviridae |
| | | | | | Olifoviridae |
| | | | | | Roniviridae |
| | | | | | Tobaniviridae |
| | | | | Picornavirales | Caliciviridae |
| | | | | | Dicistroviridae |
| | | | | | Iflaviridae |
| | | | | | Marnaviridae |
| | | | | | Picornaviridae |
| | | | | | Polycipiviridae |
| | | | | | Solinviviridae |
| | | | | Sobelivirales | Alvernaviridae |
| | | | | | Solemoviridae |
| | | | Stelpaviricetes | Stellavirales | Astroviridae |
| | | | | | Birnaviridae |
| | | | | | Permutotetraviridae |
| | Paramavirae | Artverviricota | Revtraviricetes | Blubervirales | Hepadnaviridae |
| | | | | Ortervirales | Belpaoviridae |
| | | | | | Metaviridae |
| | | | | | Pseudoviridae |
| | | | | | Retroviridae |
| | | | | | Sarthroviridae |
| Varidnaviria | Bamfordvirae | Nucleocytoviricota | Megaviricetes | Imitervirales | Mimiviridae |
| | | | | Pimascovirales | Ascoviridae |
| | | | | | Iridoviridae |
| | | | | | Marseilleviridae |
| | | | Pokkesviricetes | Asfuvirales | Asfarviridae |
| | | | | Chitovirales | Poxviridae |
| | | Preplasmiviricota | Maveriviricetes | Priklausovirales | Lavidaviridae |
| | | | Tectiliviricetes | Rowavirales | Adenoviridae |
| | Helvetiavirae | Dividoviricota | Laserviricetes | Halopanivirales | Sphaerolipoviridae |
| | | | | Ligamenvirales | Lipothrixviridae |
| | | | | | Alphasatellitidae |
| | | | | | Ampullaviridae |
| | | | | | Anelloviridae |
| | | | | | Baculoviridae |
| | | | | | Halspiviridae |
| | | | | | Hytrosaviridae |
| | | | | | Nimaviridae |
| | | | | | Nudiviridae |
| | | | | | Polydnaviridae |
| | | | | | Portogloboviridae |
| | | | | | Thaspiviridae |

| | | | Non- | Non- |
|---|---|---|---|---|
| | | | limiting, | limiting, |
| Realm | Kingdom | Phylum | exemplary genera | exemplary species |
| Duplodnaviria | Heunggongvirae | Peploviricota | Batrachovirus, Cyprinivirus, | |
| | | | Ictalurivirus, Salmonivirus | |
| | | | Cytomegalovirus, | Human |
| | | | Lymphocryptovirus, | alphaherpes- |
| | | | Macavirus, Mardivirus, | virus 1, |
| | | | Percavirus, Rhadinovirus, | Human |
| | | | Roseolovirus, Simplexvirus, | alphaherpes- |
| | | | Varicellovirus | virus 2 |
| | | | Aurivirus, Ostreavirus | |
| Monodnaviria | Loebvirae | Hofneiviricota | Thomixvirus | |
| | Sangervirae | Phixviricota | Chlamydiamicrovirus | |
| | Shotokuvirae | Cossaviricota | Bidensovirus | |
| | | | Alphapolyomavirus, | Macaca mulatta |
| | | | Betapolyomavirus, | polyomavirus 1 |
| | | | Deltapolyomavirus, | |
| | | | Gammapolyomavirus | |
| | | | Alefpapillomavirus, | Alphapapilloma- |
| | | | Alphapapillomavirus, | virus 9, |
| | | | Betapapillomavirus, | Alphapapilloma- |
| | | | Deltapapillomavirus, | virus 7 |
| | | | Dyokappapapillomavirus, | |
| | | | Dyoxipapillomavirus, | |
| | | | Dyozetapapillomavirus, | |
| | | | Gammapapillomavirus, | |
| | | | Iotapapillomavirus, | |
| | | | Kappapapillomavirus, | |
| | | | Lambdapapillomavirus, | |
| | | | Mupapillomavirus, | |
| | | | Psipapillomavirus, | |
| | | | Taupapillomavirus, | |
| | | | Thetapapillomavirus, | |
| | | | Upsilonpapillomavirus, | |
| | | | Xipapillomavirus, | |
| | | | Amdoparvovirus, | Adeno- |
| | | | Bocaparvovirus, | associated |
| | | | Chaphamaparvovirus, | dependoparvo- |
| | | | Copiparvovirus, | virus A |
| | | | Dependoparvovirus, | |
| | | | Erythroparvovirus, | |
| | | | Iteradensovirus, | |
| | | | Loriparvovirus, | |
| | | | Protoparvovirus, | |
| | | | Scindoambidensovirus, | |
| | | | Tetraparvovirus | |
| | | Cre [symbol] | Circovirus, Cyclovirus | |
| | | | Bovismacovirus, Cosmacovirus, | |
| | | | Dragsmacovirus, Drosmacovirus, | |
| | | | Huchismacovirus, Porprismacovirus | |
| | | | Torbevirus | |
| | | | Begomovirus, Mastrevirus | |
| | | | Gemycircularvirus, Gemyduguivirus, | |
| | | | Gemygorvirus, Gemykibivirus, | |
| | | | Gemykroznavirus, Gemytondvirus, | |
| | | | Gemyvongvirus | |
| Riboviria | Orhornavirae | Duplornaviricota | Megabirnavirus | |
| | | | Quadrivirus | |
| | | | Aquareovirus, Cardoreovirus, | |
| | | | Coltivirus, Cypovirus, | |
| | | | Dinovernavirus, Fijivirus, | |
| | | | Idnoreovirus, Mimoreovirus, | |
| | | | Orbivirus, Orthoreovirus, | |
| | | | Phytoreovirus, Rotavirus, | |
| | | | Seadornavirus | |
| | | Kitrinovincota | Betatetravirus, Omegatetravirus | |
| | | | Orthohepevirus, Piscihepevirus | |
| | | | Rubivirus | |
| | | | Idaeovirus | |
| | | | Alphavirus | Semliki |
| | | | | Forest virus |
| | | | Hordeivirus, Tobamovirus | |
| | | | Botrexvirus, Lolavirus, | |
| | | | Platypuvirus, Sclerodarnavirus | |
| | | | Flavivirus, Hepacivirus, | Zika virus |
| | | | Pegivirus, Pestivirus | |
| | | | Alphanodavirus, Betanodavirus | |
| | | | Sinaivirus | |
| | | | Alphacarmotetravirus | |
| | | | Polerovirus | |
| | | | Panicovirus, Tombusvirus | |
| | | Lenarviricota | Botoulivirus, Magoulivirus, | |
| | | | Ourmiavirus, Scleroulivirus | |
| | | Negarnaviricota | Yingvirus | |
| | | | Antennavirus, Hartmanivirus, | |
| | | | Mammarenavirus, Reptarenavirus | |
| | | | Lincruvirus | |
| | | | Actinovirus, Agnathovirus, | |
| | | | Loanvirus, Mobatvirus, | |
| | | | Orthohantavirus, Reptillovirus, | |
| | | | Thottimvirus | |
| | | | Shilevirus | |
| | | | Hubavirus | |
| | | | Orthonairovirus, Shaspivirus, | |
| | | | Striwavirus | |
| | | | Herbevirus, Orthobunyavirus, | |
| | | | Pacuvirus, Shangavirus | |
| | | | Feravirus, Jonvirus, | |
| | | | Orthophasmavirus, Sawastrivirus, | |
| | | | Wuhivirus | |
| | | | Bandavirus, Goukovirus, | |
| | | | Ixovirus,, Phasivirus, | |
| | | | Phlebovirus, Pidchovirus, | |
| | | | Tenuivirus, Uukuvirus, | |
| | | | Wenrivirus | |
| | | | Wumivirus | |
| | | | Tilapinevirus | |
| | | | Alphainfluenzavirus, | Influenza A |
| | | | Betainfluenzavirus, | virus, |
| | | | Deltainfluenzavirus, | Influenza B |
| | | | Gammainfluenzavirus, Isavirus, | virus, |
| | | | Quaranjavirus, Thogotovirus | Influenza D |
| | | | | virus, |
| | | | | Influenza C |
| | | | | virus |
| | | | Mivirus | |
| | | | Hexartovirus, Peropuvinis | |
| | | | Carbovirus, Cultervirus, | |
| | | | Orthobornavirus | |
| | | | Cuevavirus, Dianlovirus, | |
| | | | Ebolavirus, Marburgvirus, | |
| | | | Striavirus, Thamnovirus | |
| | | | Arlivirus | |
| | | | Berhavirus, Crustavirus, | |
| | | | Nyavirus, Orinovirus, | |
| | | | Tapwovirus | |
| | | | Henipavirus, Jeilongvirus, | Measles |
| | | | Metaavulavirus, Morbillivirus, | morbillivirus, |
| | | | Orthoavulavirus, | Avian |
| | | | Orthorubulavirus, | orthoavulavirus |
| | | | Paraavulavirus, Pararubulavirus, | 1 |
| | | | Respirovirus, Salemvirus, | |
| | | | Scoliodonvirus, Synodonvirus | |
| | | | Metapneumovirus, Orthopneumovirus | |
| | | | Almendravirus, Arurhavirus, | Indiana |
| | | | Caligrhavirus, Curiovirus, | vesiculovirus, |
| | | | Cytorhabdovirus, Ephemerovirus, | Maraba |
| | | | Hapavirus, Ledantevirus, | vesiculovirus |
| | | | Lyssavirus, Novirhabdovirus, | |
| | | | Ohlsrhavirus, Perhabdovirus, | |
| | | | Sawgrhavirus, Sigmavirus, | |
| | | | Sprivivirus, Sunrhavirus, | |
| | | | Tibrovirus, Tupavirus, | |
| | | | Vesiculovirus, | |
| | | | Sunshinevirus | |
| | | | Anphevirus | |
| | | | Yuyuevirus | |
| | | Pisuviricota | Amalgavirus, Zybavirus | |
| | | | Picobirnavirus | |
| | | | Alphaabyssovirus | |
| | | | Betaarterivinis, | |
| | | | Epsilonarterivirus,, | |
| | | | Iotaarterivirus, | |
| | | | Thetaarterivirus, | |
| | | | Alphacoronavirus, Alphaletovirus, | |
| | | | Betacoronavirus, Deltacoronavirus, | |
| | | | Gammacoronavirus | |
| | | | Pontunivirus | |
| | | | Charybnivirus, Paguronivirus | |
| | | | Cyclophivirus | |
| | | | Bolenivirus, Turrinivirus | |
| | | | Alphamesonivirus | |
| | | | Alphamononivirus | |
| | | | Chimshavirus | |
| | | | Sajorinivirus | |
| | | | Kukrinivirus | |
| | | | Okavirus | |
| | | | Bafinivirus, Bostovirus, | |
| | | | Infratovirus, Lyctovirus, | |
| | | | Oncotshavirus, Pregotovirus, | |
| | | | Sectovirus, Torovirus | |
| | | | Bavovirus, Lagovirus, | |
| | | | Minovirus, Nacovirus, | |
| | | | Nebovirus, Norovirus, | |
| | | | Recovirus, Salovirus, | |
| | | | Sapovirus, Valovirus, | |
| | | | Vesivirus | |
| | | | Aparavirus, Cripavirus, | |
| | | | Triatovirus | |
| | | | Iflavirus | |
| | | | Kusarnavirus, Locarnavirus, | |
| | | | Marnavirus, Salisharnavirus, | |
| | | | Sogarnavirus | |
| | | | Anativirus, Aphthovirus, | Enterovirus A, |
| | | | Avisivirus, Boosepivirus, | Enterovirus B, |
| | | | Cardiovirus, Cosavirus, | Enterovirus C |
| | | | Enterovirus, Fipivirus, | |
| | | | Grusopivirus, Hepatovirus, | |
| | | | Kobuvirus, Kunsagivirus, | |
| | | | Limnipivirus, Megrivirus, | |
| | | | Mischivirus, Mosavirus, | |
| | | | Parabovirus, Parechovinis, | |
| | | | Passerivirus, Potamipivirus, | |
| | | | Rabovirus, Rafivirus, | |
| | | | Rosavirus, Sapelovirus,, | |
| | | | Teschovirus, Tremovirus, | |
| | | | Chipolycivirus, Hupolycivirus, | |
| | | | Sopolycivirus | |
| | | | Invictavirus, Nyfulvavirus | |
| | | | Dinomavirus | |
| | | | Sobemovirus | |
| | | | Avastrovirus, Mamastrovirus, | |
| | | | Aquabirnavirus, Blosnavirus, | |
| | | | Entomobirnavirus, | |
| | | | Alphapermutotetravirus | |
| | | | Botybirnavirus | |
| | Paramavirae | Artverviricota | Avihepadnavirus, | Hepatitis B |
| | | | Herpetohepadnavirus, | virus |
| | | | Metahepadnavirus, | |
| | | | Orthohepadnavirus, | |
| | | | Parahepadnavirus | |
| | | | Semotivirus | |
| | | | Errantivirus, Metavirus | |
| | | | Hemivirus | |
| | | | Alpharetrovirus, Betaretrovirus, | Rous sarcoma |
| | | | Bovispumavirus, Deltaretrovirus, | virus, |
| | | | | Mouse mammary |
| | | | Epsilonretrovirus, Equispumavirus, | tumor virus, |
| | | | Felispumavirus, Gammaretrovirus, | Murine leukemia |
| | | | Lentivirus, Prosimiispumavirus, | virus, Simian |
| | | | Simiispumavirus | immunodeficiency |
| | | | | virus, Human |
| | | | | immunodeficiency |
| | | | | virus 1, Human |
| | | | | immunodeficiency |
| | | | | virus 2 |
| | | | Macronovirus | |
| Varidnaviria | Bamfordvirae | Nucleocytoviricota | Cafeteriavirus, Mimivirus | |
| | | | Ascovirus, Toursvirus | |
| | | | Chloriridovirus, | |
| | | | Lymphocystivirus, | |
| | | | Megalocytivirus, Ranavirus | |
| | | | Marseillevirus | |
| | | | Asfivirus | |
| | | | Alphaentomopoxvirus, Avipoxvirus, | Vaccinia |
| | | | Betaentomopoxvirus, Capripoxvirus, | virus |
| | | | Gammaentomopoxvirus, | |
| | | | Leporipoxvirus, | |
| | | | Orthopoxvirus, Oryzopoxvirus, | |
| | | | Parapoxvirus, | |
| | | Preplasmiviricota | Mavirus, Sputnikvirus | |
| | | | Atadenovirus, Aviadenovirus, | |
| | | | Ichtadenovirus, Mastadenovirus, | |
| | | | Siadenovirus | |
| | Helvetiavirae | Dividoviricota | Betasphaerolipovirus, | |
| | | | Gammasphaerolipovirus | |
| | | | Alphalipothrixvirus | |
| | | | Clecrusatellite | |
| | | | Ampullavirus | |
| | | | Alphatorquevirus, Betatorquevirus, | |
| | | | Gammatorquevirus, Lambdatorquevirus, | |
| | | | Alphabaculovirus, Betabaculovirus, | |
| | | | Deltabaculovirus, Gammabaculovirus | |
| | | | Salterprovirus | |
| | | | Glossinavirus, Muscavirus | |
| | | | Whispovirus | |
| | | | Alphanudivirus, Betanudivirus | |
| | | | Bracovirus, Ichnovirus | |
| | | | Alphaportoglobovirus | |
| | | | Nitmarvirus | |
| | | | Deltavirus | |
| | | | Dinodnavirus | |

[symbol]

indicates data missing or illegible when filed

Non-limiting examples of viruses that may be utilized in the present disclosure are listed in Table 14 above. Viruses utilized in the present disclosure may also include oncolytic viruses. Some of the viruses listed in Table 14 above are oncolytic viruses. Oncolytic viruses are viruses that preferentially replicate in and destroy tumor cells, compared to non-neoplastic host cells. Non-limiting examples of oncolytic viruses that may be used in the compositions presented herein include adenovirus, measles virus, poliovirus, rhinovirus, reovirus, vaccinia virus, herpes simplex virus (HSV) type 1, coxsackie virus, retrovirus, Newcastle Disease virus, vesicular stomatitis virus (VSV) and Zikavirus. Oncolytic viruses can target tumor cells indirectly by stimulating an immune response, e.g., production of cytokines and chemokines leading to the recruitment of immune cells to the tumor. Oncolytic viruses may also target tumor cells by directly infecting and lysing them. Many clinical studies evaluating oncolytic viruses, especially in the context of cancer, are ongoing. Even though oncolytic viruses are promising tools for therapeutic applications and research, some limitations remain to be overcome. Such limitations include developing resistance (e.g., due to neutralizing antibodies) and lack of targeting of oncolytic viruses to tumors. See, e.g., Martinez-Quintanilla et al., J Clin Invest. 2019; 129(4):1407-1418 and Zheng et al., Molecular Therapy—Oncolytics, Volume 15, 234-247.

In certain embodiments, the viral vector, viral particle or viral protein used in the present disclosure is derived from an enveloped virus. For example, in some embodiments, the viral vector, viral particle or viral protein used in the present disclosure is derived from a lentivirus. Lentiviral vectors can be produced according to the known methods in the art, e.g., as described in Cribbs et al., BMC Biotechnology, 13:98 (2003); Merten et al., Mol Ther Methods Clin Dev., 13 (3):16017 (2016); Durand and Cimarelli, Viruses, 3:132-159 (2011). Generation of high-titer lentivirus can be accomplished with an optimized ultracentrifuge speed during viral concentration and modified culturing conditions. In some embodiments, third-generation self-inactivating lentiviral vectors are used herein, and such vectors have been used in clinical trials to introduce genes into hematopoietic stem cells to correct primary immunodeficiency and hemoglobinopathies. In some embodiments, lentiviral vectors can be used for CAR-T gene delivery, vaccines, or research tools, e.g., to introduce genes into mature T cells to generate immunity to cancer through the delivery of chimeric antigen receptors (CARs) or cloned T-cell receptors.

In other embodiments, the viral vector, viral particle or viral protein used in the present disclosure is derived from another enveloped virus, a herpes simplex virus (HSV) (see, e.g., NCBI Accession No. NC_001806). A mature HSV virion consists of an enveloped icosahedral capsid with a viral genome consisting of a linear double-stranded DNA molecule of about 152 kb and encoding approximately 84 genes. The linear double-stranded genome is composed of a long (UL) and short (US) genomic segment that contain both essential and non-essential genes. In general, accessory genes can be individually deleted without substantially compromising virus replication in standard cell cultures. By contrast, deletion of any essential gene completely blocks productive virus infection. Each genomic segment is flanked by inverted repeats creating an internal region referred to as the joint. Several genes that regulate virus replication are located in repeat regions and are therefore diploid. Consequently, the approximately 19 kb joint region can be deleted without substantially compromising virus replication, creating a large space for insertion of one or more expression cassettes.

Examples of herpes simplex virus glycoproteins may include, but are not limited to, the glycoproteins gB, gD, gH, and gL. In some embodiments, the modified envelope alters the herpes simplex virus tissue tropism relative to a wild-type herpes simplex virus. In some embodiments, the herpes simplex virus is a herpes simplex type 1 virus (HSV-1), a herpes simplex type 2 virus (HSV-2), of any derivatives thereof.

HSV-based vectors can be constructed according the methods known in the art, e.g., as described in U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties. The manipulation of particular viral genes has led to the creation of three types of HSV-based vectors: amplicon, replication-defective, and replication-competent vectors, each of which is included in the present disclosure.

The amplicons are plasmid-derived vectors engineered to contain both the origin of HSV DNA replication (ori) and HSV cleavage-packaging recognition sequences (pac). When amplicons are transfected into mammalian cells with HSV helper functions, they are replicated, form head-to-tail linked concatamers and are then packaged into viral particles. There are two major methods currently used for producing amplicon particles, one based on infection with defective helper HSVs and the other based on transfection of HSV-1 genes, such as a set of pac-deleted overlapping cosmids or a pac-deleted and ICP27-deleted BAC-HSV-1. In some embodiments, amplicons used herein can accommodate large fragments of foreign DNA (e.g., up to 152 kb), including multiple copies of the transgene (e.g., up to 15 copies), and are non-toxic.

In some embodiments, an HSV-based vector used herein is deficient in at least one essential HSV gene, and the HSV-based vector may also comprise one or more deletions of non-essential genes. In some embodiments, the HSV-based vector is replication-deficient. Most replication-deficient HSV-based vectors contain a deletion to remove one or more intermediate-early, early, or late HSV genes to prevent replication. In other embodiments, the HSV-based vector is deficient in an immediate early gene selected from the group consisting of ICP0, ICP4, ICP22, ICP27, ICP47, and a combination thereof. In a specific embodiment, the HSV-based vector is deficient for all of ICP0, ICP4, ICP22, ICP27, and ICP47. Exemplary replication-competent vectors include NV-1020 (HSV-1), RAV9395 (HSV-2), AD-472 (HSV-2), NS-gEnull (HSV-1), and ImmunoVEX (HSV2). Exemplary replication-defective vectors include d15-29 (HSV-2), d15-29-41L (HSV-1), DISC-dH (HSV-1 and HSV-2), CJ9gD (HSV-1), TOH-OVA (HSV-1), d106 (HSV-1), d81(HSV-1), HSV-SIV d106(HSV-1), and d106 (HSV-1)

Replication-deficient HSV-based vectors are typically produced in complementing cell lines that provide gene functions not present in the replication-deficient HSV-based vectors, but required for viral propagation, at appropriate levels in order to generate high titers of viral vector stock. An exemplary cell line complements for at least one and, in some embodiments, all replication-essential gene functions not present in a replication-deficient HSV-based vector. For example, a HSV-based vector deficient in ICP0, ICP4, ICP22, ICP27, and ICP47 can be complemented by the human osteosarcoma line U2OS. The cell line can also complement non-essential genes that, when missing, reduce growth or replication efficiency (e.g., UL55). The complementing cell line can complement for a deficiency in at least one replication-essential gene function encoded by the early regions, immediate-early regions, late regions, viral packaging regions, virus-associated regions, or combinations thereof, including all HSV functions (e.g., to enable propagation of HSV amplicons, which comprise minimal HSV sequences, such as only inverted terminal repeats and the packaging signal or only ITRs and an HSV promoter). In some embodiments, the cell line is further characterized in that it contains the complementing genes in a non-overlapping fashion with the HSV-based vector, which minimizes, and practically eliminates, the possibility of the HSV-based vector genome recombining with the cellular DNA. Accordingly, the presence of replication competent HSV is minimized, if not avoided in the vector stock, which, therefore, is suitable for certain therapeutic purposes, especially gene therapy purposes. The construction of complementing cell lines involves standard molecular biology and cell culture techniques well known in the art.

HSV-based vectors can be used as attenuated vaccine, or used to deliver transgenes, e.g., to the nervous system. Exemplary therapeutic transgenes using HSV vectors are described in Manservigi et al., Open Virol J., 4:123-156 (2010), which is incorporated herein by reference in its entirety. Such transgenes include, but not limited to, FGF-2, BDNF, IL-4, IL-1ra, shRNA, neprilysin, GDNF bcl-2 erithropoietin, neurotrophic factors, preproenkephalin, hexA α subunit, β-glucoronidase, IL4, IL-10, HSV-2 ICP0PK, and preproenkephalin.

In certain embodiments, the viral vector, viral particle or viral protein used in the present disclosure is derived from a non-enveloped virus. For example, in some embodiments, the viral vector, viral particle or viral protein used in the present disclosure is derived from an adenovirus. Production of adenovirus (e.g., in HEK cells) is well known in the art. Recombinant adenovirus vectors can be constructed according to known methods in the art. See, e.g., O'Connor et al., Virology, 217(1):11-22 (1996): Hardy et al., Journal of Virology, 73(9):7835-7841 (1999). For example, adenovirus vectors can be constructed through Cre-lox recombination as described in Hardy et al., Journal of Virology, 71(3):1842-1849 (1997). In some embodiments, third-generation adenoviral vectors (also called “high capacity adenoviral vectors” (HCAds), helper-dependent or “gutless” adenoviral vectors) can be used herein to cargo sequences up to 36 kb. For example, the vector is produced in HEK293 cells that constitutively express Cre recombinase by simultaneously transducing helper virus and the HCAd genome. This allows the synthesis of adenoviral proteins by the helper virus and enables assembly of viral capsids, resulting in the packaging of HCAd genome. In some embodiments, the polynucleotide of interest, e.g., a transgene is cloned into an adenoviral vector that only contains the ITRs and a packaging signal. A helper adenoviral vector may be co-transfected into HEK cells to generate the adenoviral particle. See Lee et al., Genes and Diseases, 4(2):43-63 (2007). Adenovirus derived vectors can be used in vaccines, gene therapies, or as research tools (e.g., in vitro transduction experiments and preclinical in vivo studies).

In other embodiments, the viral vector, viral particle or viral protein used in the present disclosure is derived from another non-enveloped virus, an adeno-associated virus (AAV). More detailed description related to AAV is provided in 5.3.2.1 below.

5.3.1. Transgenes

In certain aspects, a virus particle as described and utilized herein comprises a polynucleotide that comprises a transgene. Such a transgene may encode any polypeptide or polynucleotide sequence of interest.

The term “transgene” as used in a broad sense means any heterologous nucleotide sequence that encodes a gene product. A transgene may be incorporated in a vector, e.g., for expression in a target cell, that is a cell within which transgene expression is desired. A transgene can be associated with regulatory sequences, e.g., with promoter and/or regulatory control sequences such as enhancers. It is appreciated by those of skill in the art that regulatory control sequences will be selected based on ability to promote expression of the transgene in a target cell. An example of a transgene is a nucleic acid encoding a polypeptide, for example, a therapeutic polypeptide, a polynucleotide, e.g., an inhibitory polynucleotide, for example, a siRNA or a miRNA, or a detectable marker.

In certain embodiments, the transgene may encode a sequence useful for therapeutic applications. For example, in certain embodiments, the transgene may encode an antibody, for example a monospecific, bispecific, trispecific or multispecific antibody, a single chain antibody. e.g., an ScFv, or an antigen-binding fragment of an antibody. In other embodiments, for example, the transgene may encode an enzyme. In yet other embodiments, a transgene may encode a polypeptide useful for immunotherapy applications. For example, in certain embodiments, a transgene may encode an immune checkpoint inhibitor, a chimeric antigen receptor, bi-specific T-cell engager (BITE), or a T cell receptor. In certain embodiments, for example, a transgene may encode a sequence useful for gene therapy applications, e.g., may encode a sequence useful for gene replacement, gene silencing, gene addition or gene editing applications of gene therapy. In certain embodiments, a transgene may encode a sequence useful for vaccine applications, e.g., may encode an antigen to which an immune response in a subject is to be induced (for example, an infectious agent antigen, a tumor antigen or a tumor-associated antigen).

In certain embodiments, the transgene may encode a sequence useful for manufacturing or research purposes. For example, in certain embodiments, the transgene may encode a sequence useful for increasing the success of a manufacturing process, for example, success of a cell culture process, e.g., the yield of a protein expressed by the cell culture. In particular embodiments, the transgene encodes a sequence beneficial for the propagation of a cell culture, or the stability or purification of a product, e.g., a protein product of the cell culture. In certain embodiments, the transgene encodes a detectable marker useful for research purposes. In a specific embodiment, the transgene comprises a guide RNA and/or a nucleotide sequence encoding a cas gene.

In certain embodiments, the transgene encodes a polypeptide, for example a biologically active copy of a protein, e.g., a protein useful for treating a disease or disorder. In specific embodiments, the transgene encodes two or more biologically active proteins. In certain embodiments, the transgene encodes a detectable reporter protein, such as β-lactamase, β-galactosidase (LacZ), alkaline phosphatase, thymidine kinase, secreted alkaline phosphatase (SEAP), green fluorescent protein (GFP), chloramphenicol acetyltransferase (CAT), luciferase, membrane bound proteins including, for example, CD2, CD4, CD8, the influenza hemagglutinin protein, and others well known in the art. In some embodiments, the transgene is expressed in the target cell in the subject.

In some embodiments, the proteins (e.g., therapeutic proteins) encoded by the transgene include, but are not limited to, angiogenic agents, such as vascular endothelial growth factors (VEGFs, e.g., VEGF121, VEGF165. VEGF-C, VEGF-2), glioma-derived growth factor, angiogenin, angiogenin-2; and the like; anti-angiogenic agents, such as a soluble VEGF receptor; soluble receptors, such as soluble TNF-α, receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as α-glucosidase, imiglucarase, β-glucocerebrosidase, and alglucerase; enzyme activators, such as tissue plasminogen activator; chemokines, such as 1P-10, monokine induced by interferon-gamma (Mig), Groα/IL-8, RANTES, MIP-1α, MIP-1β, MCP-1, PF-4, and the like; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin: glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin: angiotropin; fibrin; hirudin; IL-1 receptor antagonists; and the like; ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoictin (TPO), transforming growth factor alpha (TGF-α), transforming growth factor beta (TGF-β), and the like; hemophilia related clotting proteins, such as Factor VIII, Factor IX, Factor X; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In certain embodiments, the transgene encodes an AAT (alpha-1 anti-trypsin) polypeptide, an ADCC (aromatic L-amino acid decarboxylase) polypeptide, an APOE2 (apolipoprotein E2) polypeptide, an α-Gal A (galactosidase alpha) polypeptide, an AQP1 (aquaporin-1 polypeptide), a ARSB (arylsulfatase B) polypeptide, a CFTR (cystic fibrosis transmembrane conductance regulator) polypeptide, a CHM (CHM Rab Escort Protein) polypeptide, a channelrhodopsin ChrimsonR-tdTomato polypeptide, a CLN2 (ceroid lipofuscinosis, neuronal, 2) polypeptide, a CLN3 (ceroid lipofuscinosis, neuronal, 3) polypeptide, a CLN6 (ceroid lipofuscinosis, neuronal, 6) polypeptide, a CNGA3 (cyclic nucleotide gated channel subunit alpha 3) polypeptide, a CNGB3 (cyclic nucleotide gated channel subunit beta 3) polypeptide, a dysferlin polypeptide, a dystrophin polypeptide (e.g., a microdystrophin polypeptide or a miniature dystrophin polypeptide), a Factor VIII polypeptide (e.g., a B-domain deleted Factor VIII polypeptide), a Factor IX polypeptide, an Flt-1 (Fins related receptor tyrosine kinase 1) polypeptide, a G6Pase (glucose-6-phosphatase) polypeptide, a GAA (acid alpha-glucosidase) polypeptide (e.g., a secretable GAA polypeptide), a GAD (glutamate decarboxylase) polypeptide, a gamma-sarcoglycan polypeptide, a GBA1 (glucosylceramidase beta) polypeptide, a GDNF (glial cell line-derived neurotrophic factor) polypeptide, a GLA (galactosidase alpha) polypeptide, a GLB1 (galactosidase beta 1) polypeptide, a GRN (granulin precursor) polypeptide, a haSG (human alpha-sarcoglycan) polypeptide, an HTT (huntingtin) polypeptide, a human lipoprotein lipase polypeptide (e.g., human lipoprotein lipase S447X), an IDS (iduronate 2-sulfatase) polypeptide, an IFN-β (interferon beta) polypeptide, an IDUA (α-L-iduronidase) polypeptide, an MTM1 (myotubularin 1) polypeptide, a LAMP2B (lysosome-associated membrane protein 2 isoform B) polypeptide, an LDLR (low density lipoprotein receptor) polypeptide, a MERTK (Mer tyrosine kinase) polypeptide, a NAGLU (N-acetyl-alpha-glucosaminidase) polypeptide, an ND4 (NADH dehydrogenase 4) polypeptide (e.g., a G1778G ND4 polypeptide), a neurturin polypeptide, an NGF (nerve growth factor) polypeptide, an NTF3 (neurotrophin 3) polypeptide, an OTC (ornithine transcarbamylase) polypeptide, a PGBD (hydroxymethylbilane synthase) polypeptide, a PDE6B (phosphodiesterase 6B) polypeptide, a REP (Rab-escort protein) polypeptide, a REP65 (retinal pigment epithelium-specific 65) polypeptide, a RPGR (retinitis pigmentosa GTPase regulator) polypeptide, an RSI (retinoschisin 1 polypeptide), a SERCA2a (sarcoplasmic reticulum calcium ATPase) polypeptide, an SGSH (N-sulfoglucosamine sulfohydrolase) polypeptide, an SMN (survival motor neuron) polypeptide, an anti-VEGF polypeptide, a VEGF-binding polypeptide, a TNFR (tumor necrosis factor receptor) polypeptide (e.g., a TNFR:immunoglobulin (IgG1) Fc fusion polypeptide), a telomerase polypeptide, or an UGT1A1 (UDP glucuronosyltransferase family 1 member A1) polypeptide.

In certain embodiments, the transgene expresses an immune checkpoint molecule or an immune checkpoint inhibitor. For example, in certain embodiments, the transgene encodes a PD1 molecule or a PD1 inhibitor, for example, an anti-PD1 antibody, a PD-L1 molecule or a PD-L1 inhibitor for example, an anti-PD-L1 inhibitor, a TIM3 molecule or TIM3 inhibitor, for example, an anti-TIM3 antibody, a LAG3 molecule or a LAG3 inhibitor, for example, an anti-LAG3 antibody, or a CTLA4 molecule, for example, an anti-CTLA4 antibody.

In some embodiments, the transgene expresses a human polypeptide. In some embodiments, the transgene expresses a truncated polypeptide.

In other embodiments, the transgene encodes a polynucleotide, for example a therapeutic polynucleotide. In specific embodiments, the polynucleotide is an inhibitory polynucleotide that inhibits the expression or activity of a gene or mRNA. In particular embodiments, the polynucleotide is an inhibitory RNA, for example, a micro RNA (miRNA) or a silencer RNA (siRNA). In specific embodiments, the transgene encodes a gene editing system or a component of a gene editing system, e.g., a zinc-finger nuclease (ZFN), transcription activator-like effector nuclease (TALEN) or CRISPR gene editing system. In certain embodiments, the transgene encodes a CRISPR gene editing component, e.g., a CRISPR/Cas guide polynucleotide or a Cas, e.g., a Cas9, polypeptide.

In certain embodiments, the transgene encodes an inhibitor polynucleotide, for example a polynucleotide that utilizes RNAi. In some embodiments, the transgene encodes an miRNA. In some embodiments, the transgene encodes an siRNA. In certain embodiments, the inhibitor polynucleotide inhibits expression or activity of Factor VIII inhibitors, HTT (huntingtin), SOD1 (superoxide dismutase 1), VEGF (vascular endothelial growth factor), human immune deficiency virus (HIV) or herpes virus C (HVC).

In specific embodiments, the transgene encodes a polypeptide that modulates the splicing of an mRNA transcript. In specific embodiments, the transgene encodes a polypeptide that increases exon inclusion in an mRNA transcript.

In certain embodiments, the transgene is operatively linked to at least one regulatory sequence. Regulatory sequences may, for example, include ITRs, sequences for transcription initiation, modulation and/or termination. In certain embodiments, regulatory sequences may, for example, include promoter sequences, enhancer sequences, e.g., upstream enhancer sequences (USEs), RNA processing signals, e.g., splicing signals, polyadenylation signal sequences, sequences that stabilize cytoplasmic mRNA, post-transcriptional regulatory elements (PREs) and/or microRNA (miRNA) target sequences. In certain embodiments, regulatory sequences may include sequences that enhance translation efficiency (e.g., Kozak sequences), sequences that enhance protein stability, and/or sequences that enhance protein processing and/or secretion. In certain embodiments, the polynucleotide may encode regulatory miRNAs.

In certain embodiments, a regulatory sequence comprises a constitutive promoter and/or regulatory control element. In certain embodiments, a regulatory sequence comprises a regulatable promoter and/or regulatory control element. In certain embodiments, a regulatory sequence comprises a ubiquitous promoter and/or regulatory control element. In certain embodiments, a regulatory sequence comprises a cell- or tissue-specific promoter and/or regulatory control element. In certain embodiments, the regulatory control element is 5′ of the coding sequence of the transgene (that is, is present in ′5 untranslated regions; 5′ UTRs). In other embodiments, the regulatory control element is 3′ of the coding sequence of the transgene (that is, is present in ′3 untranslated regions; 3′ UTRs). In certain embodiments, the polynucleotide comprises more than one regulatory control element, for example may comprise two, three, four or five control elements. In instances wherein the polynucleotide comprises more than one control element, each control element may independently be 5′ of, e.g., may flank, within, or 3′ of, e.g., may flank, the coding sequence of the transgene.

In certain embodiments, the control element is an enhancer, for example, a CMV enhancer. In some embodiments, the control elements included direct the transcription or expression of the polynucleotide of interest in the subject in vivo. Control elements can comprise control sequences normally associated with the selected polynucleotide of interest or alternatively heterologous control sequences. Exemplary control sequences include those derived from sequences encoding mammalian or viral genes, such as neuron-specific enolase promoter, a GFAP promoter, the SV40 early promoter, mouse mammary tumor virus LTR promoter, adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, and hybrid promoters.

In certain embodiments, a promoter is not cell- or tissue-specific, e.g., the promoter is considered a ubiquitous promoter. Examples of promoter sequences that may promote expression in multiple cell or tissue types include, for example, human elongation factor 1a-subunit (EF1a), cytomegalovirus (CMV) immediate-early enhancer and/or promoter, chicken beta-actin (CBA) and its derivatives, e.g., CAG, for example, a CBA promoter with an S40 intron, beta glucuronidase (GUSB), or ubiquitin C (UBC).

In certain embodiments, a promoter sequence can promote expression in particular cell types or tissues. For example, in certain embodiments, a promoter may be a muscle-specific promoter, e.g., may be a mammalian muscle creatine kinase (MCK) promoter, mammalian desmin (DES) promoter, mammalian troponin 1 (TNNI2) promoter, or a mammalian skeletal alpha-actin (ASKA) promoter.

In other embodiments, a promoter sequence may be able to promote expression in neural cells or cell types, e.g., may be a neuron-specific enolase (NSE), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), beta-globin minigene hb2, preproenkephalin (PPE), enkephalin (Enk) or excitatory amino acid transporter 2 (EAAT2) promoter.

In yet other embodiments, a promoter sequence may promote expression in the liver, e.g., may be an alpha-1-antitrypsin (hAAT) or thyroxine binding globulin (TBG) promoter.

In certain embodiments, a promoter sequence may promote expression in cardiac tissue, e.g., may be a cardiomyocyte-specific promoter such as an MHC, cTnT, or CMV-MUC2k promoter.

In certain embodiments, the promoter is a RNA pol III promoter, for example, is a U6 promoter or an H1 promoter.

In certain instances, the regulatory sequence is a sequence that increases translation efficiency, for example is a Kozak sequence. Kozak sequences are well known and have a consensus sequence of CCR(A/G)CCAUGG, where R is a purine (adenine or guanine) three bases upstream of the start codon (ATG), which is followed by another G.

In certain embodiments, the polynucleotide may comprise at least one polyadenylation (polyA) signal sequence, which are well known in the art, and which can, for example comprise polynucleotide sequences that result in addition of a 5′-AAUAAA-3′ sequence into the mRNA transcribed from the transgene. In instances where a polyadenylation sequence is present, it is generally located between the 3′ end of the transgene coding sequence and the 5 end of the 3′ ITR. In certain embodiments, the polynucleotide further comprises a polyA upstream enhancer sequence 5′ of the polyA signal sequence.

In certain embodiments, the polynucleotide comprises an intron. In certain embodiments, the intron is present within the coding sequence of the transgene. In certain embodiments, the intron is 5′ or 3′ of the coding sequence of the transgene. In certain embodiments, the intron flanks the 5′ or 3′ terminus of the coding sequence of the transgene. In certain embodiments, the polynucleotide comprises two introns. In particular embodiments, one intron is 5′ of and one intron is 3′ of the coding sequence of the transgene. In certain embodiments, one intron flanks the 5′ terminus of the coding sequence of the transgene and the second intron flanks the 3′ terminus of the coding sequence of the transgene. In certain embodiments, the intron is an SV40 intron, for example, a 5′ UTR SV40 intron.

5.3.2. Modified AAV Compositions

In certain aspects, modified viral compositions provided herein are modified AAV compositions comprising a bridging composition as presented herein specifically bound to an AAV particle, wherein the bridging composition comprises a cell surface binding moiety and a bridging moiety that binds to the AAV particle. In certain aspects, modified viral compositions provided herein are modified AAV compositions comprising a bridging composition as presented herein specifically bound to an AAV capsid, wherein the bridging composition comprises a cell surface binding moiety and a bridging moiety that binds to the AAV particle. In certain aspects, modified viral compositions provided herein are modified AAV compositions comprising a bridging composition as presented herein specifically bound to an AAV capsid protein, e.g., a VP1, VP2 or VP3 protein, wherein the bridging composition comprises a cell surface binding moiety and a bridging moiety that binds to the AAV capsid protein, e.g., a VP1, VP2 or VP3 protein.

5.3.2.1 Adeno-Associated Virus (AAV)

Adeno-associated virus (AAV) is a well-known non-enveloped virus that is widely used in gene therapy (see, e.g., Naso et al., BioDrugs. 2017: 31(4): 317-334). Naturally occurring AAV forms a virus particle that comprises a three-dimensional capsid coat or shell (a “capsid”) made up of capsid proteins (VP1, VP2 and VP3) and, contained within the capsid, an AAV viral genome.

The modified AAV compositions presented herein may comprise any AAV composition described herein e.g., any AAV particle, capsid or capsid protein, or fragment thereof, as described herein.

In certain aspects, an AAV composition described herein may comprise an AAV particle. The terms “AAV virus particle.” “AAV viral particle,” “AAV vector” or “AAV particle” are well-known terms of art, used interchangeably herein. An “AAV particle” refers to an AAV capsid and a polynucleotide (generally DNA), which may comprise an AAV genome, a portion of an AAV genome, or a polynucleotide derived from an AAV genome (e.g., one or more ITRs), which polynucleotide optionally comprises a transgene.

An AAV particle may be referred to as a “recombinant AAV particle,” “recombinant AAV viral particle,” “recombinant AAV virus particle.” or “rAAV,” which terms as used herein refer to an AAV particle that has been genetically altered, e.g., by the deletion or other mutation of an endogenous AAV gene and/or the addition or insertion of a heterologous nucleic acid construct into the polynucleotide of the AAV particle. Thus, a recombinant AAV particle generally refers to a virus particle comprising a capsid coat or shell within which is packaged a polynucleotide sequence that comprises sequences of AAV origin and sequences not of AAV origin (i.e., a polynucleotide heterologous to AAV). This polynucleotide sequence is typically a sequence of interest for the genetic alteration of a cell.

In certain aspects, an AAV composition described herein may comprise an “AAV capsid,” “empty AAV virus particle,” empty AAV viral particle,” or “capsid,” or “empty particle” when referred to herein in the context of AAV, refers to a three-dimensional shell or coat comprising an AAV capsid protein, e.g., AAV capsid proteins VP1 and VP3, or VP1, VP2 and VP3. In certain aspects, an AAV composition described herein may comprise an AAV capsid protein or a fragment of an AAV capsid. The term “AAV capsid protein” or “AAV cap protein” as used herein refers to a protein encoded by an AAV capsid (cap) gene (e.g., VP1, VP2, and VP3) or a variant or fragment thereof. The term includes a capsid protein expressed by or derived from an AAV, e.g., a recombinant AAV, such as a chimeric AAV. For example, the term includes but not limited to a capsid protein derived from any AAV serotype such as AAV1, AAV2, AAV2i8, AAV3, AAV4, AAV5, AAV6, AAV7, AAV8, AAV9, AAV10, AAV rh10, AAV 11, AAV12, AAV13, AAV-DJ, AAV3b, AAV LK03, AAV rh74, AAV Anc81, Anc82, Anc83, Anc84, Anc110, Anc13, Anc126, or Anc127, AAV_go·1, AAV hu·37, or AAV rh·8 or a variant thereof.

5.3.2.2 AAV Serotypes

AAVs may be classified into different serotypes. An AAV serotype designation is defined primarily by the AAV capsid. AAV serotypes may also be distinguished by differences in tropism profiles and capsid protein amino acid sequence. Owing mainly to differences in capsid coats, different AAV serotypes may exhibit differing traits, such differing cellular tropism, affinity to extracellular matrix proteins, and immunogenicity. Many naturally occurring and engineered AAV serotypes are known in the art, any of which may be used as described herein.

AAVs that can be utilized herein also include chimeric AAVs and pseudotyped AAVs. By a “chimeric” AAV it is generally meant an AAV comprising capsid proteins from more than one source. i.e. more than one serotype or even more than one virus. As one nonlimiting example, the subject chimeric AAV may comprise a VP1 protein from AAV2 and a VP2 protein from AAV5, or a VP1 protein of AAV8 and a VP3 protein of AAV9. As another nonlimiting example, the subject chimeric AAV may comprise capsid proteins from AAV and capsid proteins from, e.g., bocavirus or parvovirus. See, e.g. Fakhiri et al. Mol Ther Methods & Clinical Dev 2019. By a “pseudotyped” or “hybrid” AAV it is generally meant an AAV comprising a genome flanked by ITRs that are heterologous to the AAV capsid. For example, the AAV may comprise a genome comprising ITRs from AAV2 but a capsid from another AAV, e.g. as described further below (e.g., the designation AAV2/9 refers to an AAV particle comprising AAV2 ITRs and AAV9 capsid proteins). Alternatively, the ITRs may be derived the same serotype as the capsid, e.g. AAV6 ITRs and an AAV6 capsid.

In some instances, the ITRs (see below) of an AAV particle are also used to described the AAV serotype (e.g., the designation AAV2/9 refers to an AAV particle comprising AAV2 ITRs and AAV9 capsid proteins).

The modified AAV compositions described herein may comprise an AAV particle of any serotype. The modified AAV compositions described herein may comprise an AAV capsid protein or protein fragment of any serotype.

AAV serotypes may include, for example, AAV1 (Genbank Accession No. NC_002077.1; HC000057.1), AAV2 (Genbank Accession No. NC_001401.2, JC527779.1), AAV2i8 (Asokan, A., 2010, Discov. Med. 9:399), AAV3 (Genbank Accession No. NC_001729.1), AAV3-B (Genbank Accession No. AF028705.1), AAV4 (Genbank Accession No. NC_001829.1), AAV5 (Genbank Accession No. NC_006152.1; JC527780.1), AAV6 (Genbank Accession No. AF028704.1; JC527781.1), AAV7 (Genbank Accession No. NC_006260.1; JC527782.1), AAV8 (Genbank Accession No. NC_006261.1; JC527783.1), AAV9 (Genbank Accession No AX753250.1; JC527784.1), AAV10 (Genbank Accession No AY631965.1), AAVrh10 (Genbank Accession No. AY243015.1). AAV 11 (Genbank Accession No AY631966.1). AAV12 (Genbank Accession No DQ813647.1), AAV13 (Genbank Accession No EU285562.1), AAV LK03, AAVrh74, AAV DJ (Wu Z, et al., 2006, J Virol. 80:11393)-7), AAV Anc81, Anc82, Anc83, Anc84, Anc110, Anc113, Anc126, or Anc127 (Zin, E. et al., 2016, Cell. Rep. 12:1056), AAV_go·1 (Arbetum, A. E. et al., 2005, J. Virol. 79:15238), AAV hu·37, or AAVrh8, AAVrh8R, or AAV rh·8 (Wang et al., 2010, Mol. Ther. 18:119-125, or variants thereof. The citations included in this non-limiting list provide representative genome and/or capsid protein sequences.

AAV variants that can be used herein include, for example, variants of any of the above such AAV variants as AAV1 variants, e.g., AAV comprising AAV1 variant capsid proteins, AAV2 variants, e.g., AAV comprising AAV2 variant capsid proteins. AAV3 variants, e.g., AAV comprising AAV3 variant capsid proteins, AAV3-B variants, e.g., AAV comprising AAV3-B variant capsid proteins, AAV4 variants, e.g., AAV comprising AAV4 variant capsid proteins, AAV5 variants, e.g., AAV comprising AAV5 variant capsid proteins, AAV6 variants, e.g., AAV comprising AAV6 variant capsid proteins, AAV7 variants, e.g., AAV comprising AAV7 variant capsid proteins, AAV8 variants, e.g., AAV comprising AAV8 variant capsid proteins, AAVrh8, AAVrh8R, or AAV rh·8 variants, e.g., AAV comprising AAVrh8, AAVrh8R or AAV rh·8 variant capsid proteins, AAV9 variants, e.g., AAV comprising AAV9 variant capsid proteins, AAV10 variants, e.g., AAV comprising AAV10 variant capsid proteins, AAVrh10 variants, e.g., AAV comprising AAVrh10 variant capsid proteins, AAV 11 variants, e.g., AAV comprising AAV11 variant capsid proteins, AAV12 variants, e.g., AAV comprising AAV12 variant capsid proteins, AAV13 variants, e.g., AAV comprising AAV13 variant capsid proteins, AAV LK03 variants, e.g., AAV comprising AAV LK03 variant capsid proteins, or AAVrh74 variants, e.g., AAV comprising AAVrh74 variant capsid proteins, AAV Anc81 variants, e.g., AAV comprising Anc81 variant capsid proteins, AAV Anc82 variants, e.g., AAV comprising Anc82 variant capsid proteins, AAV Anc83 variants, e.g., AAV comprising Anc83 variant capsid proteins. AAV Anc84 variants, e.g., AAV comprising Anc84 variant capsid proteins, AAV Anc10 variants, e.g., AAV comprising Anc10 variant capsid proteins, AAV Anc113 variants, e.g., AAV comprising Anc113 variant capsid proteins, AAV Anc126 variants, e.g., AAV comprising Anc126 variant capsid proteins, AAV Anc127 variants, e.g., AAV comprising Anc127 variant capsid proteins, AAV Anc127 variants, e.g., AAV comprising Anc127 variant capsid proteins, AAV hu·37 variants, e.g., AAV comprising hu·37 variant capsid proteins, or AAV_go·1 variants, e.g., AAV comprising AAV_go·1 variant capsid proteins.

5.3.2.3 AAV Particles and AAV Capsid Proteins

In certain aspects, an AAV particle comprises at least one AAV capsid protein and comprises a polynucleotide which comprises a sequence from an AAV genome or a sequence derived from an AAV genome, e.g., one or more ITRs from an AAV genome or derived therefrom, which polynucleotide optionally comprises an expression cassette that optionally comprises a transgene. Expression cassettes are well known and generally comprise one or more regulatory sequences useful or necessary for expression of a transgene in a target cell. In certain embodiments, an AAV particle comprises at least one AAV capsid protein and comprises a polynucleotide which comprises a sequence from an AAV genome or a sequence derived from an AAV genome, e.g., one or more ITRs from an AAV genome or derived therefrom, which polynucleotide comprises an expression cassette that optionally comprises a transgene. In certain embodiments, an AAV particle comprises at least one AAV capsid protein and comprises a polynucleotide which comprises a sequence from an AAV genome or a sequence derived from an AAV genome, e.g., one or more ITRs from an AAV genome or derived therefrom, which polynucleotide comprises an expression cassette that comprises a transgene. In certain embodiments, an AAV particle comprises at least one AAV capsid protein and comprises a polynucleotide which comprises a sequence from an AAV genome or a sequence derived from an AAV genome, e.g., one or more ITRs from an AAV genome or derived therefrom, which polynucleotide comprises a transgene.

In some embodiments, an AAV particle comprises AAV capsid proteins and a polynucleotide from an AAV genome or a polynucleotide derived from an AAV genome, wherein the capsid proteins and the AAV genome are from an AAV of the same serotype. In some embodiments, an AAV particle comprises AAV capsid proteins and a polynucleotide from an AAV genome or a polynucleotide derived from an AAV genome, wherein the capsid proteins and the AAV genome are from AAVs of different serotypes, i.e. the AAV virus particle is a “pseudotyped” virus.

In some embodiments, the AAV particle is an empty AAV particle.

An AAV particle comprises a capsid, comprising at least one AAV capsid protein. Naturally occurring AAV capsids comprise AAV VP1, VP2 and VP3 capsid proteins, which are each encoded by splice variants of the AAV cap gene. Typically, an AAV capsid contains approximately 60 capsid proteins form the capsid, which is thought to contain an approximate ratio of 1:1:10 VP1:VP2:VP3 proteins arranged in an icosahedral structure.

The modified AAV compositions presented herein may comprise any AAV capsid or particle that comprises any AAV capsid protein as described herein. The modified AAV compositions presented herein may comprise any AAV capsid protein as described herein.

In certain embodiments, an AAV capsid protein (e.g., VP1, VP2 and/or VP3) is a naturally occurring AAV capsid protein. In certain embodiments, an AAV capsid protein (e.g., VP1, VP2 and/or VP3) is not a naturally occurring capsid protein. In some embodiments, an AAV capsid protein (e.g., VP1, VP2 and/or VP3) is derived from a naturally occurring capsid protein.

Representative, non-limiting examples of VP1, VP2 and VP3 sequences are presented in Table 15, below. In certain embodiments, an AAV capsid protein can comprise a VP1, VP2 or VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table 15 Table, respectively. In certain embodiments, an AAV capsid protein can comprise a VP1, VP2 or VP3 capsid protein sequence of Table 15.

Likewise, in certain embodiments, an AAV particle or capsid can comprise AAV VP1, VP2 and/or VP3 capsid proteins that comprises a VP1, VP2 and/or VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table, respectively. In certain embodiments, an AAV particle or capsid can comprise a VP1, VP2 and/or VP3 capsid protein sequence of Table 15 Table.

In particular embodiments, an AAV particle can comprise AAV VP1, VP2 and VP3 capsid proteins that comprises a VP1, VP2 and VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table 15, respectively. In certain embodiments, an AAV particle can comprise a VP1, VP2 and VP3 capsid protein sequence of Table 15.

In particular embodiments, an AAV capsid can comprise an AAV capsid protein that comprises a VP1, VP2 and VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table 15, respectively. In certain embodiments, an AAV capsid can comprise a VP1, VP2 and VP3 capsid protein sequence of Table 15.

In particular embodiments, an AAV particle can comprise an AAV capsid protein that comprises a VP1, VP2 and VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table 15, respectively, wherein the VP1, VP2 and VP3 capsid proteins are of the same serotype. In certain embodiments, an AAV particle can comprise a VP1, VP2 and VP3 capsid protein sequence of Table 15, wherein the VP1, VP2 and VP3 proteins of are the same serotype.

In particular embodiments, an AAV capsid can comprise an AAV capsid protein that comprises a VP1, VP2 and VP3 capsid protein sequence having 75% or more sequence identity, for example, 80%, 85%, 90%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of Table 15, respectively, wherein the VP1, VP2 and VP3 capsid proteins are of the same serotype. In certain embodiments, an AAV capsid can comprise a VP1, VP2 and VP3 capsid protein sequence of Table 15, wherein the VP1, VP2 and VP3 proteins of are the same serotype.

In some embodiments, the AAV capsid protein is a VP1 capsid protein. In other embodiments, the AAV capsid protein is a VP2 capsid protein. In other embodiments, the AAV capsid protein is a VP3 capsid protein. In some embodiments, the AAV particle or capsid comprises a VP1 capsid protein, a VP2 capsid protein and/or a VP3 capsid protein. In other embodiments, the AAV particle or capsid comprises a VP1 capsid protein, a VP2 capsid protein and a VP3 capsid protein. In some embodiments, the AAV particle or capsid comprises a VP1 capsid protein, a VP2 capsid protein and/or a VP3 capsid protein, wherein the capsid proteins of the AAV particle or capsid are of the same serotype. In other embodiments, the AAV particle or capsid comprises a VP1 capsid protein, a VP2 capsid protein and a VP3 capsid protein, wherein the capsid proteins of the AAV particle are of the same serotype.

In specific embodiments, the capsid protein is an AAV1 capsid protein. In other specific embodiments, the capsid protein is an AAV2 capsid protein. In other specific embodiments, the capsid protein is an AAV2i8 capsid protein. In other specific embodiments, the capsid protein is an AAV3 capsid protein. In other specific embodiments, the capsid protein is an AAV3b capsid protein. In other specific embodiments, the capsid protein is an AAV4 capsid protein. In other specific embodiments, the capsid protein is an AAV5 capsid protein. In other specific embodiments, the capsid protein is an AAV6 capsid protein. In other specific embodiments, the capsid protein is an AAV7 capsid protein. In other specific embodiments, the capsid protein is an AAV8 capsid protein. In other specific embodiments, the capsid protein is an AAV9 capsid protein. In other specific embodiments, the capsid protein is an AAV10 capsid protein. In other specific embodiments, the capsid protein is an AAV11 capsid protein. In other specific embodiments, the capsid protein is an AAV12 capsid protein. In other specific embodiments, the capsid protein is an AAV13 capsid protein. In other specific embodiments, the capsid protein is an AAV-DJ capsid protein. In other specific embodiments, the capsid protein is an AAV LK03 capsid protein. In other specific embodiments, the capsid protein is an AAV rh10 capsid protein. In other specific embodiments, the capsid protein is an AAV rh74 capsid protein. In other specific embodiments, the capsid protein is an AAV Anc81, Anc82, Anc83, Anc84, Anc110, Anc113, Anc126, or Anc127 capsid protein. In other specific embodiments, the capsid protein is an AAV_go·1 capsid protein. In other specific embodiments, the capsid protein is an AAV hu·37 capsid protein. In other specific embodiments, the capsid protein is an AAV rh·8 capsid protein. In some embodiments, the capsid protein provided herein is derived from an AAV1, AAV2, AAV2i8, AAV3, AAV4, AAV5, AAV6, AAV7, AAV8, AAV9, AAV10, AAV rh10, AAV11, AAV12, AAV13, AAV-DJ, AAV3b, AAV LK03, AAV rh74, AAV Anc81, Anc82, Anc83, Anc84, Anc110, Anc113, Anc126, or Anc127, AAV_go·1, AAV hu·37, or AAV rh·8 capsid protein. In specific embodiments, the capsid protein has an amino acid sequence that is at least 80%, at least 85%, at least 90%, at least 95%, at least 96%, at least 97%, at least 98%, at least 99%, at least 99.9%, or 100% identical to the amino acid sequence of an AAV1, AAV2, AAV2i8, AAV3, AAV4, AAV5, AAV6, AAV7, AAV8, AAV9, AAV10, AAV rh10, AAV 11, AAV12, AAV13, AAV-DJ, AAV3b, AAV LK03, AAV rh74, AAV Anc81, Anc82, Anc83, Anc84, Anc110, Anc113, Anc126, or Anc127, AAV hu·37, AAV rh·8, or AAV_go·1 capsid protein.

In some aspects, the capsid protein is a variant capsid protein. A variant capsid protein may comprise one or more mutations, e.g. amino acid substitutions, amino acid deletions, and heterologous peptide insertions, compared to a corresponding reference capsid protein such as the naturally occurring parental capsid protein, i.e. the capsid protein from which it was derived. In some embodiments the amino acid sequence of the AAV capsid protein is identical to the amino acid sequence of the wild type, or reference, or parent AAV capsid protein except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, e.g., except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residue substitutions. For example, in certain embodiments, a variant AAV capsid protein is identical to the amino acid sequence of any of the VP1, VP2 or VP3 amino acid sequences of Table 15, except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, e.g., except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residue substitutions.

In some embodiments, the capsid protein or AAV particle described herein may be a chimeric capsid protein or AAV particle, respectively, comprising a protein sequence of two or more AAV serotype capsid proteins or particles, respectively, as discussed above.

In some embodiments, the capsid protein is an AAV1 VP1 capsid protein or a variant thereof. In some embodiments, the capsid protein is an AAV1 VP2 capsid protein or a variant thereof. In other embodiments, the capsid protein is an AAV1 VP3 capsid protein or a variant thereof.

In some embodiments, the capsid protein is an AAV2 VP1 capsid protein or a variant thereof. In some embodiments, the capsid protein is an AAV2 VP2 capsid protein or a variant thereof. In other embodiments, the capsid protein is an AAV2 VP3 capsid protein or a variant thereof.

In some embodiments, the capsid protein is an AAV3 VP1 capsid protein or a variant thereof. In some embodiments, the capsid protein is an AAV3 VP2 capsid protein or a variant thereof. In other embodiments, the capsid protein is an AAV3 VP3 capsid protein or a variant thereof.

In some embodiments, the capsid protein is an AAV4 VP1 capsid protein or a variant thereof. In some embodiments, the capsid protein is an AAV4 VP2 capsid protein or a variant thereof. In other embodiments, the capsid protein is an AAV4 VP3 capsid protein or a variant thereof.

In some embodiments, the capsid protein is an AAV5 VP1 capsid protein or a variant thereof. In some embodiments, the capsid protein is an AAV5 VP2 capsid protein or a variant thereof. In other embodiments, the capsid protein is an AAV5 VP3 capsid protein or a variant thereof.

In some embodiments, the capsid protein is an AAV6 VP1 capsid protein or a variant thereof. In some embodiments, the capsid protein is an AAV6 VP2 capsid protein or a variant thereof. In other embodiments, the capsid protein is an AAV6 VP3 capsid protein or a variant thereof.

In some embodiments, the capsid protein is an AAV7 VP1 capsid protein or a variant thereof. In some embodiments, the capsid protein is an AAV7 VP2 capsid protein or a variant thereof. In other embodiments, the capsid protein is an AAV7 VP3 capsid protein or a variant thereof.

In some embodiments, the capsid protein is an AAV8 VP1 capsid protein or a variant thereof. In some embodiments, the capsid protein is an AAV8 VP2 capsid protein or a variant thereof. In other embodiments, the capsid protein is an AAV8 VP3 capsid protein or a variant thereof.

In some embodiments, the capsid protein is an AAV9 VP1 capsid protein or a variant thereof. In some embodiments, the capsid protein is an AAV9 VP2 capsid protein or a variant thereof. In other embodiments, the capsid protein is an AAV9 VP3 capsid protein or a variant thereof.

In some embodiments, the capsid protein is an AAV10 VP1 capsid protein or a variant thereof. In some embodiments, the capsid protein is an AAV10 VP2 capsid protein or a variant thereof. In other embodiments, the capsid protein is an AAV10 VP3 capsid protein or a variant thereof.

In some embodiments, the capsid protein is an AAV11 VP1 capsid protein or a variant thereof. In some embodiments, the capsid protein is an AAV 11 VP2 capsid protein or a variant thereof. In other embodiments, the capsid protein is an AAV11 VP3 capsid protein or a variant thereof.

In some embodiments, the capsid protein is an AAV12 VP1 capsid protein or a variant thereof. In some embodiments, the capsid protein is an AAV12 VP2 capsid protein or a variant thereof. In other embodiments, the capsid protein is an AAV12 VP3 capsid protein or a variant thereof.

In some embodiments, the capsid protein is an AAV13 VP1 capsid protein or a variant thereof. In some embodiments, the capsid protein is an AAV13 VP2 capsid protein or a variant thereof. In other embodiments, the capsid protein is an AAV13 VP3 capsid protein or a variant thereof.

In some embodiments, the capsid protein is an AAV-DJ VP1 capsid protein or a variant thereof. In some embodiments, the capsid protein is an AAV-DJ VP2 capsid protein or a variant thereof. In other embodiments, the capsid protein is an AAV-DJ VP3 capsid protein or a variant thereof.

In some embodiments, the capsid protein is an AAV LK03 VP1 capsid protein or a variant thereof. In some embodiments, the capsid protein is an AAV LK03 VP2 capsid protein or a variant thereof. In other embodiments, the capsid protein is an AAV LK03 VP3 capsid protein or a variant thereof.

In some embodiments, the capsid protein is an AAV rh10 VP1 capsid protein or a variant thereof. In some embodiments, the capsid protein is an AAV rhl0 VP2 capsid protein or a variant thereof. In other embodiments, the capsid protein is an AAV rh10 VP3 capsid protein or a variant thereof.

In some embodiments, the capsid protein is an AAV rh74 VP1 capsid protein or a variant thereof. In some embodiments, the capsid protein is an AAV rh74 VP2 capsid protein or a variant thereof. In other embodiments, the capsid protein is an AAV rh74 VP3 capsid protein or a variant thereof.

In some embodiments, the capsid protein is an AAV Anc8l, Anc82, Anc83, Anc84, Anc110, Anc113, Anc126, or Anc127VP1 capsid protein or a variant thereof. In some embodiments, the capsid protein is an AAV Anc81, Anc82, Anc83, Anc84, Anc110, Anc113, Anc126, or Anc127 VP2 capsid protein or a variant thereof. In other embodiments, the capsid protein is an AAV Anc81, Anc82, Anc83, Anc84, Anc110, Anc113, Anc126, or Anc127 VP3 capsid protein or a variant thereof.

In some embodiments, the capsid protein is an AAV hu·37 VP1 capsid protein or a variant thereof. In some embodiments, the capsid protein is an AAV hu·37 VP2 capsid protein or a variant thereof. In other embodiments, the capsid protein is an AAV hu·37 VP3 capsid protein or a variant thereof.

In some embodiments, the capsid protein is an AAV rh·8 VP1 capsid protein or a variant thereof. In some embodiments, the capsid protein is an AAV rh·8 VP2 capsid protein or a variant thereof. In other embodiments, the capsid protein is an AAV rh·8 VP3 capsid protein or a variant thereof.

In some embodiments, the capsid protein is an AAV_go·1 VP1 capsid protein or a variant thereof. In some embodiments, the capsid protein is an AAV_go·1 VP2 capsid protein or a variant thereof. In other embodiments, the capsid protein is an AAV_go·1 VP3 capsid protein or a variant thereof.

In some embodiments, the capsid protein is an AAV2i8 VP1 capsid protein or a variant thereof. In some embodiments, the capsid protein is an AAV2i8 VP2 capsid protein or a variant thereof. In other embodiments, the capsid protein is an AAV2i8 VP3 capsid protein or a variant thereof.

In certain embodiments, an AAV conjugate or fusion provided herein comprises a fragment of an AAV particle or an AAV capsid protein.

In some embodiments, an AAV capsid protein fragment is a fragment of an AAV VP1 protein that is not a full-length AAV VP2 or VP3 protein. In other embodiments, an AAV capsid protein fragment is a fragment of an AAV VP2 protein that is not a full-length AAV VP3 protein. In certain embodiments, an AAV capsid protein fragment is a polypeptide of 10-700 amino acids, 10-600 amino acids, 10-500 amino acids, 10-400 amino acids, 10-300 amino acids, 10-200 amino acids, 10-100 amino acids, 50-700 amino acids, 50-600 amino acids, 50-500 amino acids, 50-400 amino acids, 50-300 amino acids, 50-200 amino acids, 100-700 amino acids, 100-600 amino acids, 100-500 amino acids, 100400 amino acids, 100-300 amino acids, 100-200 amino acids, 200-700 amino acid, 200-600 amino acids, 200-500 amino acids, 200-400 amino acids, 200-300 amino acids, 300-700 amino acids, 300-600 amino acids, 300-500 amino acids, 300-400 amino acids, 400-700 amino acids, 400-600 amino acids, 400-500 amino acids, 10-50 amino acids, 50-100 amino acids, 100-150 amino acids, 150-200 amino acids, 200-250 amino acids, 300-350 amino acids, 350400 amino acids, 400-450 amino acids, 500-550 amino acids, 550-600 amino acids, 600-650 amino acids, 650-700 amino acids, or 700-730 amino acids. In some embodiments, a fragment of an AAV particle is a protein complex of 10-50 kDa, 50-100 kDa, 100-150 kDa, 200-250 kDa, 250-300 kDa, 300-350 kDa, 350-400 kDa, 400450 kDa, 450-500 kDa, 500-550 kDa, 550-600 kDa, 600-650 kDa, 650-700 kDa, 700-750 kDa, 750-800 kDa, 800-850 kDa, 850-900 kDa, 900-950 kDa, 950-1000 kDa, 1000-1500 kDa, 1500-2000 kDa, 2000-2500 kDa, 2500-3000 kDa, 3000-3500 kDa, or 35004000 kDa in size. In some embodiments, a fragment of an AAV particle contains 1-5 VP capsid proteins (e.g., VP1, VP2 or VP3 capsid proteins or a combination thereof), 5-10 VP capsid proteins (e.g., VP1, VP2 or VP3 capsid proteins or a combination thereof), 10-15 VP capsid proteins (e.g., VP1, VP2 or VP3 capsid proteins or a combination thereof), 15-20 VP capsid proteins (e.g., VP1, VP2 or VP3 capsid proteins or a combination thereof), 20-25 VP capsid proteins (e.g., VP1, VP2 or VP3 capsid proteins or a combination thereof), 25-30 VP capsid proteins (e.g., VP1, VP2 or VP3 capsid proteins or a combination thereof), 30-35 VP capsid proteins (e.g., VP1, VP2 or VP3 capsid proteins or a combination thereof), 35-40 VP capsid proteins (e.g., VP1, VP2 or VP3 capsid proteins or a combination thereof), 40-45 VP capsid proteins (e.g., VP1, VP2 or VP3 capsid proteins or a combination thereof), 45-50 VP capsid proteins (e.g., VP1, VP2 or VP3 capsid proteins or a combination thereof), 50-55 VP capsid proteins (e.g., VP1, VP2 or VP3 capsid proteins or a combination thereof), or 55-60 VP capsid proteins (e.g., VP1, VP2 or VP3 capsid proteins or a combination thereof), or 1-55, 1-50, 1-45, 1-40, 1-35, 1-30, 1-25, 1-20, 1-15, 1-10, 5-55, 5-50, 5-45, 5-40, 5-35, 5-30, 5-25, 5-20, 5-15, 10-55, 10-50, 10-45, 1040, 10-35, 10-30, 10-25, 10-20, 15-55, 15-50, 15-45, 1540, 15-35, 15-30, 15-25, 20-55, 20-50, 2545, 2540, 25-35, 30-55, 30-50, 30-45, 3040, 40-55, 40-50, or 45-55 VP capsid proteins (e.g., VP1, VP2 or VP3 capsid proteins).

In certain specific embodiments wherein an AAV conjugate or fusion described herein comprises an AAV capsid protein or capsid protein fragment, such a capsid protein may be fused or conjugated to an agent such as an immunoglobulin Fc region. In such embodiments, it is understood that the AAV conjugate fusion may be conjugated to either or both portions (AAV capsid protein portion or agent portion).

5.3.3. AAV Particle Polynucleotides

In certain aspects, an AAV particle (used herein interchangeably with “AAV vector”) comprises a polynucleotide comprising a sequence from an AAV genome or a polynucleotide derived from an AAV genome (e.g., one or more AAV or AAV-derived ITRs), and an expression cassette, which may further optionally comprise a transgene. In certain aspects, an AAV particle comprises a polynucleotide comprising a sequence from an AAV genome or a polynucleotide derived from an AAV genome (e.g., one or more AAV or AAV-derived ITRs), and a transgene.

Naturally occurring AAV is typically a single-stranded DNA virus. Its linear genome is 4681 nucleotides long and contains a rep gene which encodes the Rep40, Rep52, Rep68 and Rep78 proteins required for replication and packaging of the viral genome, a cap gene, encoding the VP1, VP2 and VP3 capsid proteins (via splice variants), and an aap gene which encodes the assembly activating protein (AAP) (Naso et al., BioDrugs. 2017; 31(4); 317-334). The rep and cap genes are usually found adjacent to each other in the viral genome and they are generally conserved among AAV serotypes. Rep78 and Rep68 are transcribed from the p5 promoter, and Rep 52 and Rep40 are transcribed from the p19 promoter. The cap genes are transcribed from the p40 promoter. In general, the AAV genome comprising the Rep, Cap, and aap genes are flanked by 3′ and 5′ inverted terminal repeats (ITRs). The terms “inverted terminal repeat” and “ITR” sequence are well known terms of art that refer to relatively short sequences found at the termini of viral genomes which are in opposite orientation. ITR sequences suitable for use with AAV are well known in the art, and are usually an approximately 145-nucleotide sequence that is present at both termini of the native single-stranded AAV genome, or a derivative thereof. The outermost nucleotides, e.g., the outermost 125 nucleotides, of the ITR can be present in either of two alternative orientations, leading to heterogeneity between different AAV genomes and between the two ends of a single AAV genome. The outermost nucleotides, e.g., the outermost 125 nucleotides, also contain several shorter regions of self-complementarity (designated A, A′, B, B′, C, C′ and D regions), allowing intrastrand base-pairing to occur within this portion of the ITR.

In certain embodiment, the ITRs are approximately 145 nucleotides in length. In certain embodiments, the ITRS are 145 nucleotides in length. In certain embodiments, the ITRS are 100-150 nucleotides in length. In certain embodiments, the ITRS are 140-150 nucleotides in length. In certain embodiments, the ITRS are 140-150 nucleotides in length. In certain embodiments, the ITRS are 146-150 nucleotides in length.

In specific embodiments, the ITRs are of AAV origin or are derived from AAV. For example, in certain embodiments, e.g., ITRs of any of AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV11, AAV12, AAV13, AAV LK03, AAVrh74, AAV DJ, AAV Anc81, Anc82, Anc83, Anc84, Anc110, Anc13, Anc126, or Anc127, AAV hu·37, AAV rh·8, AAV_go·1, AAV LK03 serotype.

AAV particles provided herein also include pseudotyped AAV particles. By a “pseudotyped” or “hybrid” AAV particle it is generally meant an AAV comprising a genome flanked by ITRs that are heterologous to the AAV capsid. For example, the AAV may comprise a genome comprising ITRs from AAV2 but a capsid from another AAV, e.g. AAV9. In such instances, the nomenclature AAVx/y may be used, where the “x” represents the ITR source and the “y” represents the capsid source. Thus, for example, AAV2/9 refers to an AAV particle comprising AAV2 ITRs and AAV9 capsid proteins, while AAV6/3B would refer to an AAV particle comprising AAV6 ITRs and an AAV3B capsid protein. Traditionally, in the absence of any ITR designation, the ITRs from AAV2 are being used.

The ITRs may be derived from the same serotype as the capsid, or a derivative thereof. The ITR may be of a different serotype than the capsid. In certain embodiments, the polynucleotide of the AAV particle has two AAV ITRs, e.g., a 5′ AAV ITR and a ′3 AAV ITR. In certain embodiments, the ITRs are of the same serotype as one another. In another embodiment, the ITRs are of different serotypes. For example, in certain embodiments, an AAV particle comprises AAV2 ITRs and an AAV6 capsid (AAV 2/6), AAV2 ITRs and an AAV7 capsid (AAV 2/7), AAV2 ITRs and an AAV8 capsid (AAV 2/8), or AAV2 ITRs and an AAV9 capsid (AAV 2/9). In general, the capsid comprises three proteins, VP1, VP2 and VP3, with VP2 an VP3 being truncated version of VP1 so having sequences that are also comprised by VP1. Generally, the amino acid sequence of VP1 defines the serotype of the capsid. Thus, for example, if the VP1 capsid protein encodes for an AAV2 VP1 protein, AAV will be of the AAV2 serotype, whereas if the VP1 capsid protein encodes an AAV9 VP1 protein, the AAV will be of the AAV9 serotype.

In a specific embodiment, the ITRs are derived from AAV1. In a specific embodiment, the ITRs are derived from AAV2. In a specific embodiment, the ITRs are derived from AAV3. In a specific embodiment, the ITRs are derived from AAV3b. In a specific embodiment, the ITRs are derived from AAV4. In a specific embodiment, the ITRs are derived from AAV5. In a specific embodiment, the ITRs are derived from AAV6. In a specific embodiment, the ITRs are derived from AAV7. In a specific embodiment, the ITRs are derived from AAV8. In a specific embodiment, the ITRs are derived from AAVrh8, AAVrh8R or AAV rh·8. In a specific embodiment, the ITRs are derived from AAV9. In a specific embodiment, the ITRs are derived from AAV10. In a specific embodiment, the ITRs are derived from AAV11. In a specific embodiment, the ITRs are derived from AAV12. In a specific embodiment, the ITRs are derived from AAV13. In a specific embodiment, the ITRs are derived from AAV rh10. In a specific embodiment, the ITRs are derived from AAV rh74. In the absence of any ITR designation, it is assumed that the ITRs of AAV2 or a variant thereof are being employed.

The polynucleotide may comprise a sequence from an AAV genome or a polynucleotide derived from an AAV genome (e.g., one or more AAV or AAV-derived ITRs), and may optionally comprise a transgene. In some-embodiments, the polynucleotide is self-complimentary.

The total length of the polynucleotide should not exceed the total packaging capacity of the AAV capsid. For example, in certain embodiments, the polynucleotide comprises a transgene, a 5′ ITR and a 3′ ITR, and the total length of the polynucleotide containing such sequences should not exceed the total packaging capacity of the capsid. In other embodiments, the polynucleotide comprises a transgene, regulatory sequences, a 5′ ITR and a 3′ ITR, and the total length of the polynucleotide should not exceed the packaging capacity of the capsid. In certain embodiments, the polynucleotide comprises about 2 to 5 kilobases (kb) about 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4,3, 4.4, 4.45, 4.5, 4.6, 4.61, 4.62, 4.63, 4.64, 4.65, 4.66, 4.67, 4.68, 4.69, 4.7, 4.75, 4.8, 4.85 or 4.9 kb.

In certain embodiments, the desired polynucleotide, for example, a polynucleotide comprising a transgene encoding a polypeptide of interest, a 5′ ITR and a 3′ ITR, or a polynucleotide comprising a transgene, regulatory sequences, a 5′ ITR and a 3′ ITR, exceeds the total packaging capacity of the capsid. In such embodiments, a first modified AAV composition described herein may comprise a first AAV particle comprising a polynucleotide that comprises a first portion of the desired polynucleotide, and a second modified AAV composition described herein may comprise a second AAV particle comprising a polynucleotide that comprises a second portion of the desired polynucleotide. In such embodiments, the nucleotide sequence of the polynucleotide of the AAV particle and the nucleotide sequence of the polynucleotide of the second AAV particle are designed such that recombination between the two polynucleotides results in the formation of the desired polynucleotide. Thus, by transducing a cell with the first modified AAV composition and the second AAV composition the desired polynucleotide may be constructed intracellularly via recombination between the polynucleotide of the first and second AAV particles.

In other embodiments, a first modified AAV composition described herein may comprise a first AAV particle comprising a polynucleotide that encodes a first portion of the polypeptide of interest, and a second modified AAV composition described herein may comprise a second AAV particle comprising a polynucleotide that encodes a second portion of the polypeptide of interest. In such embodiments, the first portion of the polypeptide of interest and the second portion of the polypeptide of interest are designed to be joined via a split intein system to produce the polypeptide of interest. Thus, by transducing a cell with the first modified AAV composition and the second AAV composition the polypeptide may be constructed intracellularly after the transgenes have been expressed first portion and the second portion of the polypeptide, via protein splicing between the two polypeptides.

The polynucleotide may comprise a sequence from an AAV genome or a polynucleotide derived from an AAV genome (e.g., one or more AAV or AAV-derived ITRs), and may optionally comprise a transgene.

In certain embodiments, an AAV particle comprises a polynucleotide that comprises a sequence heterologous to an AAV genome. In certain embodiments, the heterologous sequence comprises an expression cassette which comprises nucleotide sequences useful for expression of a transgene in a target cell. In certain embodiments, the heterologous sequence comprises the expression cassette further comprises nucleotide sequences useful for expression of the transgene. In certain embodiments, the heterologous sequence comprises a transgene. In particular embodiments, an AAV particle comprises a polynucleotide that comprises a transgene and at least one inverted terminal repeat (“ITR”), for example a flanking ITR 5′ of the transgene (a “5′ ITR”), a flanking ITR 3′ of the transgene (a “3′ ITR”), or a 5′ ITR and a 3′ ITR. ITRs include sequences which can be complementary and symmetrically arranged. The AAV genome may either be single-stranded (ssAAV) or self-complementary (scAAV), as when the heterologous polynucleotide is engineered to be complementary to itself.

In certain embodiments, an AAV particle comprises a polynucleotide that comprises a transgene and at least one inverted terminal repeat, for example a 5′ ITR”), a 3′ ITR”, or a 5′ ITR and a 3′ ITR, and at least one regulatory sequence. Regulatory sequences may, for example, include sequences for transcription initiation, modulation and/or termination. In certain embodiments, regulatory sequences may, for example, include promoter sequences, enhancer sequences, e.g., upstream enhancer sequences (USEs), RNA processing signals, e.g., splicing signals, polyadenylation signal sequences, sequences that stabilize cytoplasmic mRNA, post-transcriptional regulatory elements (PREs), e.g., Woodchuck PREs (WPREs) and/or microRNA (miRNA) target sequences. In certain embodiments, regulatory sequences may include sequences that enhance translation efficiency (e.g., Kozak sequences), sequences that enhance protein stability, and/or sequences that enhance protein processing and/or secretion. In certain embodiments, the polynucleotide may encode regulatory miRNAs.

In certain embodiments, a regulatory sequence comprises a constitutive promoter and/or regulatory control element. In certain embodiments, a regulatory sequence comprises a regulatable promoter and/or regulatory control element. In certain embodiments, a regulatory sequence comprises a ubiquitous promoter and/or regulatory control element. In certain embodiments, a regulatory sequence comprises a cell- or tissue-specific promoter and/or regulatory control element. In certain embodiments, the regulatory control element is 5′ of the coding sequence of the transgene (that is, is present in ′5 untranslated regions; 5′ UTRs). In other embodiments, the regulatory control element is 3′ of the coding sequence of the transgene (that is, is present in ′3 untranslated regions; 3′ UTRs). In certain embodiments, the polynucleotide comprises more than one regulatory control element, for example may comprise two, three, four or five control elements. In instances wherein the polynucleotide comprises more than one control element, each control element may independently be 5′ of, e.g., may flank, within, or 3′ of, e.g., may flank, the coding sequence of the transgene.

In certain embodiments, the control element is an enhancer, for example, a CMV enhancer. In some embodiments, the control elements included in the present polynucleotide direct the transcription or expression of the polynucleotide of interest in the subject in vivo. Control elements can comprise control sequences normally associated with the selected polynucleotide of interest or alternatively heterologous control sequences. Exemplary control sequences include those derived from sequences encoding mammalian or viral genes, such as neuron-specific enolase promoter, a GFAP promoter, the SV40 early promoter, mouse mammary tumor virus LTR promoter, adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, and hybrid promoters.

In certain embodiments, a promoter is not cell- or tissue-specific, e.g., the promoter is considered a ubiquitous promoter. Examples of promoter sequences that may promote expression in multiple cell or tissue types include, for example, human elongation factor 1a-subunit (EF1a), cytomegalovirus (CMV) immediate-early enhancer and/or promoter, chicken beta-actin (CBA) and its derivatives, e.g., CAG, for example, a CBA promoter with an S40 intron, beta glucuronidase (GUSB), or ubiquitin C (UBC).

In certain embodiments, a promoter sequence can promote expression in particular cell types or tissues. For example, in certain embodiments, a promoter may be a muscle-specific promoter, e.g., may be a mammalian muscle creatine kinase (MCK) promoter, mammalian desmin (DES) promoter, mammalian troponin 1 (TNN12) promoter, or a mammalian skeletal alpha-actin (ASKA) promoter.

In other embodiments, a promoter sequence may be able to promote expression in neural cells or cell types, e.g., may be a neuron-specific enolase (NSE), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), beta-globin minigene hb2, preproenkephalin (PPE), enkephalin (Enk) or excitatory amino acid transporter 2 (EAAT2) promoter.

In yet other embodiments, a promoter sequence may promote expression in the liver, e.g., may be an alpha-1-antitrypsin (hAAT) or thyroxine binding globulin (TBG) promoter.

In certain embodiments, a promoter sequence may promote expression in cardiac tissue, e.g., may be a cardiomyocyte-specific promoter such as an MHC, cTnT, or CMV-MUC2k promoter.

In certain embodiments, the promoter is a RNA pol III promoter, for example, is a U6 promoter or an H1 promoter.

In certain instances, the regulatory sequence is a sequence that increases translation efficiency, for example is a Kozak sequence. Kozak sequences are well known and have a consensus sequence of CCR(A/G)CCAUGG, where R is a purine (adenine or guanine) three bases upstream of the start codon (ATG), which is followed by another G. Generally, when a Kozak sequence is present, it is located in the 5′ UTR.

In certain embodiments, the polynucleotide may comprise at least one polyadenylation (polyA) signal sequence, which are well known in the art, and which can, for example comprise polynucleotide sequences that result in addition of a 5′-AAUAAA-3′ sequence into the mRNA transcribed from the transgene. In instances where a polyadenylation sequence is present, it is generally located between the 3′ end of the transgene coding sequence and the 5′ end of the 3′ ITR. In certain embodiments, the polynucleotide further comprises a polyA upstream enhancer sequence 5′ of the polyA signal sequence.

In certain embodiments, the polynucleotide comprises an intron. In certain embodiments, the intron is present within the coding sequence of the transgene. In certain embodiments, the intron is 5′ or 3′ of the coding sequence of the transgene. In certain embodiments, the intron flanks the 5′ or 3′ terminus of the coding sequence of the transgene. In certain embodiments, the polynucleotide comprises two introns. In particular embodiments, one intron is 5′ of and one intron is 3′ of the coding sequence of the transgene. In certain embodiments, one intron flanks the 5′ terminus of the coding sequence of the transgene and the second intron flanks the 3′ terminus of the coding sequence of the transgene. In certain embodiments, the intron is an SV40 intron, for example, a 5′ UTR SV40 intron.

In other embodiments, the polynucleotide comprises a filler, or stuffer, sequence which may be included to improve packaging efficiency and expression. In certain embodiments, a stuffer or filler sequence may, for example comprise an albumin and/or alpha-1 antitrypsin sequence.

In specific embodiments, an AAV particle comprises a polynucleotide that comprises at least one ITR, a transgene, a promoter sequence, and at least one enhancer sequence. In specific embodiments, an AAV particle comprises a polynucleotide that comprises at least one ITR, a transgene, a promoter sequence, at least one enhancer sequence and at least one intron. In specific embodiments, an AAV particle comprises a polynucleotide that comprises, in 5′ to 3′ order, a 5′ ITR, an enhancer sequence, e.g., a CMV enhancer sequence, a promoter, and a transgene coding sequence. In specific embodiments, an AAV particle comprises a polynucleotide that comprises, in 5′ to 3′ order, a 5′ ITR, an enhancer sequence, e.g., a CMV enhancer sequence, a promoter, an intron and a transgene coding sequence.

In specific embodiments, an AAV particle comprises a polynucleotide that comprises at least one ITR, a transgene, a polyA signal sequence and, optionally, a polyA upstream enhancer sequence. In specific embodiments, an AAV particle comprises a polynucleotide that comprises at least one ITR, a transgene, a polyA signal sequence, optionally, a polyA upstream enhancer sequence and at least one intron.

In specific embodiments, an AAV particle comprises a polynucleotide that comprises, in 5′ to 3′ order, a transgene coding sequence, a polyA signal sequence and a 3′ ITR. In specific embodiments, an AAV particle comprises a polynucleotide that comprises, in 5′ to 3′ order, a transgene coding sequence, a polyA upstream enhancer sequence, a polyA signal sequence and a 3′ ITR. In specific embodiments, an AAV particle comprises a polynucleotide that comprises, in 5′ to 3′ order, a transgene coding sequence, an intron, a polyA signal sequence and a 3′ ITR. In specific embodiments, an AAV particle comprises a polynucleotide that comprises, in 5′ to 3′ order, a transgene coding sequence, an intron, a polyA upstream enhancer sequence, a polyA signal sequence and a 3′ ITR.

In specific embodiments, an AAV particle comprises a polynucleotide that comprises, in 5′ to 3′ order, a 5′ ITR, an enhancer sequence, e.g., a CMV enhancer sequence, a promoter, optionally an intron, a transgene coding sequence, optionally an intron, optionally a polyA upstream enhancer sequence, a polyA signal sequence and a 3′ ITR. In certain embodiments, the polynucleotide comprises two introns, which may be the same or different from each other.

TABLE 15
Representative AAV VP1, VP2 and VP3 amino acid sequences

| SEQ ID | | |
|---|---|---|
| NO. | AAV | Sequence |
| 54 | AAV1 | MAADGYLPDWLEDNLSEGIREWWDLKPGAPKPKANQQKQDDGR |
| | (UniProt | GLVLPGYKYLGPFNGLDKGEPVNAADAAALEHDKAYDQQLKAG |
| | Q9WBP8_9VIRU) | DNPYLRYNHADAEFQERLQEDTSFGGNLGRAVFQAKKRVLEPLGL |
| | VP1: amino | VEEGAKTAPGKKRPVEQSPQEPDSSSGIGKTGQQPAKKRLNFGQT |
| | acids 1-736; | GDSESVPDPQPLGEPPATPAAVGPTTMASGGGAPMADNNEGADG |
| | VP2: amino | VGNASGNWHCDSTWLGDRVITTSTRTWALPTYNNHLYKQISSAST |
| | acids 138-736 | GASNDNHYFGYSTPWGYFDFNRFHCHFSPRDWQRLINNNWGFRP |
| | VP3: amino | KRLNFKLFNIQVKEVTTNDGVTTIANNLTSTVQVFSDSEYQLPYVL |
| | acids 203-736 | GSAHQGCLPPFPADVFMIPQYGYLTLNNGSQAVGRSSFYCLEYFPS |
| | | QMLRTGNNFTFSYTFEEVPFHSSYAHSQSLDRLMNPLIDQYLYYLN |
| | | RTQNQSGSAQNKDLLFSRGSPAGMSVQPKNWLPGPCYRQQRVSK |
| | | TKTDNNNSNFTWTGASKYNLNGRESIINPGTAMASHKDDEDKFFP |
| | | MSGVMIFGKESAGASNTALDNVMITDEEEIKATNPVATERFGTVA |
| | | VNFQSSSTDPATGDVHAMGALPGMVWQDRDVYLQGPIWAKIPHT |
| | | DGHFHPSPLMGGFGLKNPPPQILIKNTPVPANPPAEFSATKFASFITQ |
| | | YSTGQVSVEIEWELQKENSKRWNPEVQYTSNYAKSANVDFTVDN |
| | | NGLYTEPRPIGTRYLTRPL |
| 55 | AAV2 | MAADGYLPDWLEDTLSEGIRQWWKLKPGPPPPKPAERHKDDSRG |
| | (UniProt: | LVLPGYKYLGPFNGLDKGEPVNEADAAALEHDKAYDRQLDSGDN |
| | P03135-1) | PYLKYNHADAEFQERLKEDTSFGGNLGRAVFQAKKRVLEPLGLVE |
| | VP1: amino | EPVKTAPGKKRPVEHSPVEPDSSSGTGKAGQQPARKRLNFGQTGD |
| | acids 1-735 | ADSVPDPQPLGQPPAAPSGLGTNTMATGSGAPMADNNEGADGVG |
| | VP2: amino | NSSGNWHCDSTWMGDRVITTSTRTWALPTYNNHLYKQISSQSGAS |
| | acids 138-735 | NDNHYFGYSTPWGYFDFNRFHCHFSPRDWQRLINNNWGFRPKRL |
| | VP3: amino | NFKLFNIQVKEVTQNDGTTTIANNLTSTVQVFTDSEYQLPYVLGSA |
| | acids 203-735 | HQGCLPPFPADVFMVPQYGYLTLNNGSQAVGRSSFYCLEYFPSQM |
| | | LRTGNNFTFSYTFEDVPFHSSYAHSQSLDRLMNPLIDQYLYYLSRT |
| | | NTPSGTTTQSRLQFSQAGASDIRDQSRNWLPGPCYRQQRVSKTSA |
| | | DNNNSEYSWTGATKYHLNGRDSLVNPGPAMASHKDDEEKFFPQS |
| | | GVLIFGKQGSEKTWDIEKVMITDEEEIRTTNPVATEQYGSVSTNL |
| | | QRGNRQAATADVNTQGVLPGMVWQDRDVYLQGPIWAKIPHTDG |
| | | HFHPSPLMGGFGLKHPPPQILIKNTPVPANPSTTFSAAKFASFITQYS |
| | | TGQVSVEIEWELQKENSKRWNPEIQYTSNYNKSVNVDFTVDTNGV |
| | | YSEPRPIGTRYLTRNL |
| 56 | AAV3B | MAADGYLPDWLEDNLSEGIREWWALKPGVPQPKANQQHQDNRR |
| | (Uniprot | GLVLPGYKYLGPGNGLDKGEPVNEADAAALEHDKAYDQQLKAG |
| | O56139_9VIRU) | DNPYLKYNHADAEFQERLQEDTSFGGNLGRAVFQAKKRILEPLGL |
| | VP1: amino | VEEAAKTAPGKKRPVDQSPQEPDSSSGVGKSGKQPARKRLNFGQT |
| | acids 1-736 | GDSESVPDPQPLGEPPAAPTSLGSNTMASGGGAPMADNNEGADGV |
| | VP2: amino | GNSSGNWHCDSQWLGDRVITTSTRTWALPTYNNHLYKQISSQSGA |
| | acids 138-736 | SNDNHYFGYSTPWGYFDFNRFHCHFSPRDWQRLINNNWGFRPKK |
| | VP3: amino | LSFKLFNIQVKEVTQNDGTTTIANNLTSTVQVFTDSEYQLPYVLGS |
| | acids 203-736 | AHQGCLPPFPADVFMVPQYGYLTLNNGSQAVGRSSFYCLEYFPSQ |
| | | MLRTGNNFQFSYTFEDVPFHSSYAHSQSLDRLMNPLIDQYLYYLN |
| | | RTQGTTSGTTNQSRLLFSQAGPQSMSLQARNWLPGPCYRQQRLSK |
| | | TANDNNNSNFPWTAASKYHLNGRDSLVNPGPAMASHKDDEEKFF |
| | | PMHGNLIFGKEGTTASNAELDNVMITDEEEIRTTNPVATEQYGTVA |
| | | NNLQSSNTAPTTRTVNDQGALPGMVWQDRDVYLQGPIWAKIPHT |
| | | DGHFHPSPLMGGFGLKHPPPQIMIKNTPVPANPPTTFSPAKFASFIT |
| | | QYSTGQVSVEIEWELQKENSKRWNPEIQYTSNYNKSVNVDFTVDT |
| | | NGVYSEPRPIGTRYLTRNL |
| 57 | AAV4 | MTDGYLPDWLEDNLSEGVREWWALQPGAPKPKANQQHQDNARG |
| | (Uniprot | LVLPGYKYLGPGNGLDKGEPVNAADAAALEHDKAYDQQLKAGD |
| | O41855_9VIRU) | NPYLKYNHADAEFQQRLQGDTSFGGNLGRAVFQAKKRVLEPLGL |
| | VP1: amino | VEQAGETAPGKKRPLIESPQQPDSSTGIGKKGKQPAKKKLVFEDET |
| | acids 1-734 | GAGDGPPEGSTSGAMSDDSEMRAAAGGAAVEGGQGADGVGNAS |
| | VP2: amino | GDWHCDSTWSEGHVTTTSTRTWVLPTYNNHLYKRLGESLQSNTY |
| | acids 137-734 | NGFSTPWGYFDFNRFHCHHSPRDWQRLINNNWGMRPKAMRVKIF |
| | VP3: amino | NIQVKEVTTSNGETTVANNLTSTVQIFADSSYELPYVMDAGQEGSL |
| | acids 197-734 | PPFPNDVFMVPQYGYCGLVTGNTSQQQTDRNAFYCLEYFPSQMLR |
| | | TGNNFEITYSFEKVPFHSMYAHSQSLDRLMNPLIDQYLWGLQSTTT |
| | | GTTLNAGTATTNFTKLRPTNFSNFKKNWLPGPSIKQQGFSKTANQ |
| | | NYKIPATGSDSLIKYETHSTLDGRWSALTPGPPMATAGPADSKFSN |
| | | SQLIFAGPKQNGNTATVPGTLIFTSEEELAATNATDTDMWGNLPG |
| | | GDQSNSNLPTVDRLTALGAVPGMVWQNRDIYYQGPIWAKIPHTD |
| | | GHFHPSPLIGGFGLKHPPPQIFIKNTPVPANPATTFSSTPVNSFITQYS |
| | | TGQVSVQIDWEIQKERSKRWNPEVQFTSNYGQQNSLLWAPDAAG |
| | | KYTEPRAIGTRYLTHHL |
| 58 | AAV5 | MSFVDHPPDWLEEVGEGLREFLGLEAGPPKPKPNQQHQDQARGL |
| | (Uniprot | VLPGYNYLGPGNGLDRGEPVNRADEVAREHDISYNEQLEAGDNP |
| | Q9YIJ1_9VIRU) | YLKYNHADAEFQEKLADDTSFGGNLGKAVFQAKKRVLEPFGLVE |
| | VP1: amino | EGAKTAPTGKRIDDHFPKRKKARTEEDSKPSTSSDAEAGPSGSQQL |
| | acids 1-724; | QTPAQPASSLGADTMSAGGGGPLGDNNQGADGVGNASGDWHCDS |
| | VP2: amino | TWMGDRVVTKSTRTWVLPSYNNHQYREIKSGSVDGSNANAYFGY |
| | acids 137-724; | STPWGYFDFNRFHSHWSPRDWQRLINNYWGFRPRSLRVKIFNIQV |
| | VP3: amino | KEVTVQDSTTTIANNLTSTVQVFTDDDYQLPYVVGNGTEGCLPAF |
| | acids 193-724. | PPQVFTLPQYGYATLNRDNTENPTERSSFFCLEYFPSKMLRTGNNF |
| | | EFTYNFEEVPFHSSFAPSQNLFKLANPLVDQYLYRFVSTNNTGGVQ |
| | | FNKNLAGRYANTYKNWFPGPMGRTQGWNLGSGVNRASVSAFAT |
| | | TNRMELEGASYQVPPQPNGMTNNLQGSNTYALENTMIFNSQPANP |
| | | GTTATYLEGNMLITSESETQPVNRVAYNVGGQMATNNQSSTTAPA |
| | | TGTYNLQEIVPGSVWMERDVYLQGPIWAKIPETGAHFHPSPAMGG |
| | | FGLKHPPPMMLIKNTPVPGNITSFSDVPVSSFITQYSTGQVTVEME |
| | | WELKKENSKRWNPEIQYTNNYNDPQFVDFAPDSTGEYRTTRPIGT |
| | | RYLTRPL |
| 59 | AAV6 | MAADGYLPDWLEDNLSEGIREWWDLKPGAPKPKANQQKQDDGR |
| | (Uniprot | GLVLPGYKYLGPFNGLDKGEPVNAADAAALEHDKAYDQQLKAG |
| | O56137_9VIRU) | DNPYLRYNHADAEFQERLQEDTSFGGNLGRAVFQAKKRVLEPFGL |
| | VP1: amino | VEEGAKTAPGKKRPVEQSPQEPDSSSGIGKTGQQPAKKRLNFGQT |
| | acids 1-736 | GDSESVPDPQPLGEPPATPAAVGPTTMASGGGAPMADNNEGADG |
| | VP2: amino | VGNASGNWHCDSTWLGDRVITTSTRTWALPTYNNHLYKQISSAST |
| | acids 138-736 | GASNDNHYFGYSTPWGYFDFNRFHCHFSPRDWQRLINNNWGFRP |
| | VP3: amino | KRLNFKLFNIQVKEVTTNDGVTTIANNLTSTVQVFSDSEYQLPYVL |
| | acids 203-736 | GSAHQGCLPPFPADVFMTPQYGYLTLNNGSQAVGRSSFYCLEYFPS |
| | | QMLRTGNNFTFSYTFEDVPFHSSYAHSQSLDRLMNPLIDQYLYYL |
| | | NRTQNQSGSAQNKDLLFSRGSPAGMSVQPKNWLPGPCYRQQRVS |
| | | KTKTDNNNSNFTWTGASKYNLNGRESIINPGTAMASHKDDKDKFF |
| | | PMSGVMIFGKESAGASNTALDNVMITDEEEIKATNPVATERFGTV |
| | | AVNLQSSSTDPATGDVHVMGALPGMVWQDRDVYLQGPIWAKIPH |
| | | TDGHFHPSPLMGGFGLKHPPPQILIKNTPVPANPPAEFSATKFASFTT |
| | | QYSTGQVSVEIEWELQKENSKRWNPEVQYTSNYAKSANVDFTVD |
| | | NNGLYTEPRPIGTRYLTRPL |
| 60 | AAV7 | MAADGYLPDWLEDNLSEGIREWWDLKPGAPKPKANQQKQDNGR |
| | (Uniprot | GLVLPGYKYLGPFNGLDKGEPVNAADAAALEHDKAYDQQLKAG |
| | Q8JQG0_9VIRU) | DNPYLRYNHADAEFQERLQEDTSFGGNLGRAVFQAKKRVLEPLGL |
| | VP1: amino | VEEGAKTAPAKKRPVEPSPQRSPDSSTGIGKKGQQPARKRLNFGQT |
| | acids 1-737 | GDSESVPDPQPLGEPPAAPSSVGSGTVAAGGGAPMADNNEGADG |
| | VP2: amino | VGNASGNWHCDSTWLGDRVITTSTRTWALPTYNNHLYKQISSETA |
| | acids 138-737 | GSTNDNTYFGYSTPWGYFDFNRFHCHFSPRDWQRLINNNWGFRPK |
| | VP3: amino | KLRFKLFNIQVKEVTTNDGVTTIANNLTSTIQVFSDSEYQLPYVLGS |
| | acids 204-737 | AHQGCLPPFPADVFMIPQYGYLTLNNGSQSVGRSSFYCLEYFPSQM |
| | | LRTGNNFEFSYSFEDVPFHSSYAHSQSLDRLMNPLIDQYLYYLART |
| | | QSNPGGTAGNRELQFYQGGPSTMAEQAKNWLPGPCFRQQRVSKT |
| | | LDQNNNSNFAWTGATKYHLNGRNSLVNPGVAMATHKDDEDRFF |
| | | PSSGVLIFGKTGATNKTTLENVLMTNEEEIRPTNPVATEEYGIVSSN |
| | | LQAANTAAQTQVVNNQGALPGMVWQNRDVYLQGPIWAKIPHTD |
| | | GNFHPSPLMGGFGLKHPPPQILIKNTPVPANPPEVFTPAKFASFITQY |
| | | STGQVSVEIEWELQKENSKRWNPEIQYTSNFEKQTGVDFAVDSQG |
| | | VYSEPRPIGTRYLTRNL |
| 61 | AAV8 | MAADGYLPDWLEDNLSEGIREWWALKPGAPKPKANQQKQDDGR |
| | (Uniprot | GLVLPGYKYLGPFNGLDKGEPVNAADAAALEHDKAYDQQLQAG |
| | Q8JQF8_9VIRU) | DNPYLRYNHADAEFQERLQEDTSFGGNLGRAVFQAKKRVLEPLGL |
| | VP1: amino | VEEGAKTAPGKKRPVEPSPQRSPDSSTGIGKKGQQPARKRLNFGQT |
| | acids 1-738; | GDSESVPDPQPLGEPPAAPSGVGPNTMAAGGGAPMADNNEGADG |
| | VP2: amino | VGSSSGNWHCDSTWLGDRVITTSTRTWALPTYNNHLYKQISNGTS |
| | acids 138-738; | GGATNDNTYFGYSTPWGYFDFNRFHCHFSPRDWQRLINNNWGFR |
| | VP3: amino | PKRLSFKLFNIQVKEVTQNEGTKTIANNLTSTIQVFTDSEYQLPYVL |
| | acids 204-738. | GSAHQGCLPPFPADVFMIPQYGYLTLNNGSQAVGRSSFYCLEYFPS |
| | | QMLRTGNNFQFTYTFEDVPFHSSYAHSQSLDRLMNPLIDQYLYYL |
| | | SRTQTTGGTANTQTLGFSQGGPNTMANQAKNWLPGPCYRQQRVS |
| | | TTTGQNNNSNFAWTAGTKYHLNGRNSLANPGIAMATHKDDEERF |
| | | FPSNGILIFGKQNAARDNADYSDVMLTSEEEIKTTNPVATEEYGIV |
| | | ADNLQQQNTAPQIGTVNSQGALPGMVWQNRDVYLQGPIWAKIPH |
| | | TDGNFHPSPLMGGFGLKHPPPQILIKNTPVPADPPTTFNQSKLNSFIT |
| | | QYSTGQVSVEIEWELQKENSKRWNPEIQYTSNYYKSTSVDFAVNT |
| | | EGVYSEPRPIGTRYLTRNL |
| 62 | AAV9 | MAADGYLPDWLEDNLSEGIREWWALKPGAPQPKANQQHQDNAR |
| | (Uniprot | GLVLPGYKYLGPGNGLDKGEPVNAADAAALEHDKAYDQQLKAG |
| | Q6JC40_9VIRU) | DNPYLKYNHADAEFQERLKEDTSFGGNLGRAVFQAKKRLLEPLGL |
| | VP1: amino | VEEAAKTAPGKKRPVEQSPQEPDSSAGIGKSGAQPAKKRLNFGQT |
| | acids 1-736 | GDTESVPDPQPIGEPPAAPSGVGSLTMASGGGAPVADNNEGADGV |
| | VP2: amino | GSSSGNWHCDSQWLGDRVTTTSTRTWALPTYNNHLYKQTSNSTSG |
| | acids 138-736 | GSSNDNAYFGYSTPWGYFDFNRFHCHFSPRDWQRLINNNWGFRP |
| | VP3: amino | KRLNFKLFNIQVKEVTDNNGVKTIANNLTSTVQVFTDSDYQLPYV |
| | acids 203-736 | LGSAHEGCLPPFPADVFMIPQYGYLTLNDGSQAVGRSSFYCLEYFP |
| | | SQMLRTGNNFQFSYEFENVPFHSSYAHSQSLDRLMNPLIDQYLYYL |
| | | SKTINGSGQNQQTLKFSVAGPSNMAVQGRNYIPGPSYRQQRVSTT |
| | | VTQNNNSEFAWPGASSWALNGRNSLMNPGPAMASHKEGEDRFFP |
| | | LSGSLIFGKQGTGRDNVDADKVMITNEEEIKTTNPVATESYGQVAT |
| | | NHQSAQAQAQTGWVQNQGILPGMVWQDRDVYLQGPIWAKIPHT |
| | | DGNFHPSPLMGGFGMKHPPPQILIKNTPVPADPPTAFNKDKLNSFIT |
| | | QYSTGQVSVEIEWELQKENSKRWNPEIQYTSNYYKSNNVEFAVNT |
| | | EGVYSEPRPIGTRYLTRNL |
| 63 | AAV12 | MAADGYLPDWLEDNLSEGIREWWALKPGAPQPKANQQHQDNGR |
| | UniProt: | GLVLPGYKYLGPFNGLDKGEPVNEADAAALEHDKAYDKQLEQGD |
| | A9RAI0_9VIRU | NPYLKYNHADAEFQQRLATDTSFGGNLGRAVFQAKKRILEPLGLV |
| | VP1: amino | EEGVKTAPGKKRPLEKTPNRPTNPDSGKAPAKKKQKDGEPADSAR |
| | acids 1-742 | RTLDFEDSGAGDGPPEGSSSGEMSHDAEMRAAPGGNAVEAGQGA |
| | VP2: amino | DGVGNASGDWHCDSTWSEGRVTTTSTRTWVLPTYNNHLYLRIGT |
| | acids 138-742 | TANSNTYNGFSTPWGYFDFNRFHCHFSPRDWQRLINNNWGLRPKS |
| | VP3: amino | MRVKIFNIQVKEVTTSNGETTVANNLTSTVQIFADSTYELPYVMDA |
| | acids 206-742 | GQEGSFPPFPNDVFMVPQYGYCGVVTGKNQNQTDRNAFYCLEYF |
| | | PSQMLRTGNNFEVSYQFEKVPFHSMYAHSQSLDRMMNPLLDQYL |
| | | WHLQSTTTGNSLNQGTATTTYGKITTGDFAYYRKNWLPGACIKQQ |
| | | KFSKNANQNYKIPASGGDALLKYDTHTTLNGRWSNMAPGPPMAT |
| | | AGAGDSDFSNSQLIFAGPNPSGNTTTSSNNLLFTSEEEIATTNPRDT |
| | | DMFGQIADNNQNATTAPHLANLDAMGIVPGMVWQNRDIYYQGPI |
| | | WAKVPHTDGHFHPSPLMGGFGLKHPPPQIFIKNTPVPANPNTTFSA |
| | | ARINSFLTQYSTGQVAVQIDWEIQKEHSKRWNPEVQFTSNYGTQN |
| | | SMLWAPDNAGNYHELRAIGSRFLTHHL |
| 64 | AAVrh8 | MAADGYLPDWLEDNLSEGIREWWDLKPGAPKPKANQQKQDDGR |
| | (Uniprot | GLVLPGYKYLGPFNGLDKGEPVNAADAAALEHDKAYDQQLKAG |
| | Q808Y3_9VIRU) | DNPYLRYNHADAEFQERLQEDTSFGGNLGRAVFQAKKRVLEPLGL |
| | VP1: amino | VEEGAKTAPGKKRPVEQSPQEPDSSSGIGKTGQQPAKKRLNFGQT |
| | acids 1-736 | GDSESVPDPQPLGEPPAAPSGLGPNTMASGGGAPMADNNEGADG |
| | VP2: amino | VGNSSGNWHCDSTWLGDRVITTSTRTWALPTYNNHLYKQISNGTS |
| | acids 138-736 | GGSTNDNTYFGYSTPWGYFDFNRFHCHFSPRDWQRLINNNWGFRP |
| | VP3: amino | KRLNFKLFNIQVKEVTTNEGTKTIANNLTSTVQVFTDSEYQLPYVL |
| | acids 203-736 | GSAHQGCLPPFPADVFMVPQYGYLTLNNGSQALGRSSFYCLEYFP |
| | | SQMLRTGNNFQFSYTFEDVPFHSSYAHSQSLDRLMNPLIDQYLYYL |
| | | VRTQTTGTGGTQTLAFSQAGPSSMANQARNWVPGPCYRQQRVST |
| | | TTNQNNNSNFAWTGAAKFKLNGRDSLMNPGVAMASHKDDDDRF |
| | | FPSSGVLIFGKQGAGNDGVDYSQVLITDEEEIKATNPVATEEYGAV |
| | | AINNQAANTQAQTGLVHNQGVIPGMVWQNRDVYLQGPIWAKIPH |
| | | TDGNFHPSPLMGGFGLKHPPPQILIKNTPVPADPPLTFNQAKLNSFI |
| | | TQYSTGQVSVEIEWELQKENSKRWNPEIQYTSNYYKSTNVDFAVN |
| | | TEGVYSEPRPIGTRYLTRNL |
| 65 | AAVrh10 | MAADGYLPDWLEDNLSEGIREWWDLKPGAPKPKANQQKQDDGR |
| | (Uniprot | GLVLPGYKYLGPFNGLDKGEPVNAADAAALEHDKAYDQQLKAG |
| | Q808W5_9VIRU) | DNPYLRYNHADAEFQERLQEDTSFGGNLGRAVFQAKKRVLEPLGL |
| | VP1: amino | VEEGAKTAPGKKRPVETSPQRSPDSSTGIGKKGQQPAKKRLNFGQT |
| | acids 1-738; | GDSESVPDPQPIGEPPAGPSGLGSGTMAAGGGAPMADNNEGADGV |
| | VP2: amino | GSSSGNWHCDSTWLGDRVITTSTRTWALPTYNNHLYKQISNGTSG |
| | acids 138-738; | GSTNDNTYFGYSTPWGYFDFNRFHCHFSPRDWQRLINNNWGFRPK |
| | VP3: amino | RLNFKLFNIQVKEVTQNEGTKTIANNLTSTTQVFTDSEYQLPYVLGS |
| | acids 204-738. | AHQGCLPPFPADVFMIPQYGYLTLNNGSQAVGRSSFYCLEYFPSQ |
| | | MLRTGNNFEFSYQFEDVPFHSSYAHSQSLDRLMNPLIDQYLYYLSR |
| | | TQSTGGTAGTQQLLFSQAGPNNMSAQAKNWLPGPCYRQQRVSTT |
| | | LSQNNNSNFAWTGATKYHLNGRDSLVNPGVAMATHKDDEERFFP |
| | | SSGVLMFGKQGAGKDNVDYSSVMLTSEEEIKTTNPVATEQYGVV |
| | | ADNLQQQNAAPIVGAVNSQGALPGMVWQNRDVYLQGPIWAKIPH |
| | | TDGNFHPSPLMGGFGLKHPPPQILIKNTPVPADPPTTFSQAKLASFIT |
| | | QYSTGQVSVEIEWELQKENSKRWPEIQYTSNYYKSTNVDFAVNT |
| | | DGTYSEPRPIGTRYLTRNL |
| 66 | AAV110 | MAADGYLPDWLEDNLSEGIREWWDLKPGAPKPKANQQKQDDGR |
| | (Uniprot | GLVLPGYKYLGPFNGLDKGEPVNAADAAALEHDKAYDQQLKAG |
| | A0A0K1P7V4_ | DNPYLRYNHADAEFQERLQEDTSFGGNLGRAVFQAKKRVLEPLGL |
| | 9VIRU) | VEEGAKTAPGKKRPVEQSPQEPDSSSGIGKTGQQPAKKRLNFGQT |
| | VP1: amino | GDSESVPDPQPLGEPPAAPSGVGSNTMASGGGAPMADNNEGADG |
| | acids 1-736 | VGNSSGNWHCDSTWLGDRVITTSTRTWALPTYNNHLYKQISNGTS |
| | VP2: amino | GGSTNDNTYFGYSTPWGYFDFNRFHCHFSPRDWQRLINNNWGFRP |
| | acids 138-736 | KRLNFKLFNIQVKEVTTNEGTKTIANNLTSTVQVFTDSEYQLPYVL |
| | VP3: amino | GSAHQGCLPPFPADVFMIPQYGYLTLNNGSQAVGRSSFYCLEYFPS |
| | acids 203-736 | QMLRTGNNFQFSYTFEDVPFHSSYAHSQSLDRLMNPLIDQYLYYLS |
| | | RTQTTGTAGTQTLQFSQAGPSSMANQARNWVPGPCYRQQRVSTT |
| | | TNQNNNSNFAWTGATKYHLNGRDSLMNPGVAMASHKDDEDRFF |
| | | PSSGVLIFGKQGAGNDNVDYSQVMITNEEEIKTTNPVATEEYGAV |
| | | ATNNQSANTQAQTGLVHNQGVLPGMVWQNRDVYLQGPIWAKIP |
| | | HTDGNFHPSPLMGGFGLKHPPPQILIKNTPVPADPPTTFNQAKLNSF |
| | | ITQYSTGQVSVEIEWELQKENSKRWNPEIQYTSNYYKSTNVDFAV |
| | | NTEGVYSEPRPIGTRYLTRNL |
| 67 | LK03 | MAADGYLPDWLEDNLSEGIREWWALQPGAPKPKANQQHQDNAR |
| | (NCBI | GLVLPGYKYLGPGNGLDKGEPVNAADAAALEHDKAYDQQLKAG |
| | Accession | DNPYLKYNHADAEFQERLKEDTSFGGNLGRAVFQAKKRLLEPLGL |
| | AGT20780.1) | VEEAAKTAPGKKRPVDQSPQEPDSSSGVGKSGKQPARKRLNFGQT |
| | VP1: amino | GDSESVPDPQPLGEPPAAPTSLGSNTMASGGGAPMADNNEGADGV |
| | acids 1-736; | GNSSGNWHCDSQWLGDRVITTSTRTWALPTYNNHLYKQISSQSGA |
| | VP2: amino | SNDNHYFGYSTPWGYFDFNRFHCHFSPRDWQRLINNNVVGFRPKK |
| | acids 138-736; | LSFKLFNIQVKEVTQNDGTTTIANNLTSTVQVFTDSEYQLPYVLGS |
| | VP3: amino | AHQGCLPPFPADVFMVPQYGYLTLNNGSQAVGRSSFYCLEYFPSQ |
| | acids 203-736. | MLRTGNNFQFSYTFEDVPFHSSYAHSQSLDRLMNPLIDQYLYYLN |
| | | RTQGTTSGTTNQSRLLFSQAGPQSMSLQARNWLPGPCYRQQRLSK |
| | | TANDNNNSNFPWTAASKYHLNGRDSLVNPGPAMASHKDDEEKFF |
| | | PMHGNLIFGKEGTTASNAELDNVMITDEEEIRTTNPVATEQYGTVA |
| | | NNLQSSNTAPTTRTVNDQGALPGMVWQDRDVYLQGPIWAKIPHT |
| | | DGHFHPSPLMGGFGLKHPPPQIMTKNTPVPANPPTTFSPAKFASFIT |
| | | QYSTGQVSVEIEWELQKENSKRWNPEIQYTSNYNKSVNVDFTVDT |
| | | NGVYSEPRPIGTRYLTRPL |

### 5.4. Pharmaceutical Compositions

Also provided herein are pharmaceutical compositions comprising modified viral compositions (e.g., as described herein), comprising a viral composition bound to a bridging composition that comprises a bridging moiety covalently linked to a cell surface receptor binding moiety, and a pharmaceutically acceptable carrier.

In some embodiments, the pharmaceutical composition is a modified AAV composition including an AAV particle, and a pharmaceutically acceptable carrier. In some embodiments, the modified AAV composition includes an AAV capsid protein, and a pharmaceutically acceptable carrier.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.: Rowe et al., Eds.; The Pharmaceutical Press and the American Pharmaceutical Association: 2009. Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

Modified viral compositions, for example, modified viral compositions comprising an AAV particle or an AAV capsid protein to be used in a pharmaceutical composition described herein may be purified by a method known in the art. A pharmaceutical composition is substantially free from contaminants of e.g., the expression systems.

In certain embodiments, provided herein are pharmaceutical compositions comprising a therapeutically effective amount of one or more of the modified viral compositions and a pharmaceutically acceptable carrier.

In certain embodiments, the pharmaceutical compositions contain therapeutically effective amounts of one or more of the modified viral compositions, and optionally one or more additional prophylactic or therapeutic agents, in a pharmaceutically acceptable carrier. Pharmaceutical compositions may be useful for the prevention, treatment, management or amelioration of a disease or disorder described herein or one or more symptoms thereof.

Pharmaceutical carriers suitable for administration of the modified viral compositions include any such carriers known to those skilled in the art to be suitable for the particular mode of administration.

The modified viral compositions can be formulated as the sole pharmaceutically active ingredient in the composition or can be combined with other active ingredients.

In certain embodiments, the modified viral composition is formulated into one or more suitable pharmaceutical preparations, such as solutions, suspensions, sustained release formulations or elixirs in sterile solutions or suspensions for parenteral administration.

In pharmaceutical compositions provided herein, a modified viral composition, e.g., a modified AAV composition, described herein may be mixed with a suitable pharmaceutical carrier. The concentration of the modified viral composition in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of the composition is dissolved, suspended, dispersed or otherwise mixed in a selected carrier at an effective concentration such that the treated condition is relieved, prevented, or one or more symptoms are ameliorated.

Concentrations of the modified viral composition in a pharmaceutical composition provided herein will depend on, e.g., the physicochemical characteristics of the modified viral composition, the dosage schedule, and amount administered as well as other factors known to those of skill in the art.

In some embodiments, the modified viral composition described above is a modified AAV composition (e.g., as described herein).

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. The modified viral composition, e.g., the modified AAV composition, is, in certain embodiments, formulated and administered in unit-dosage forms or multiple-dosage forms. Unit-dose forms as used herein refers to physically discrete units suitable for human or animal (e.g., mammal) subjects and packaged individually as is known in the art, e.g., genome copies (GC) or vector genomes (vg). Each unit-dose contains a predetermined quantity of a modified viral composition, e.g., an modified AAV composition, sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the modified viral compositions, e.g., modified AAV compositions, provided herein are in a liquid pharmaceutical formulation. Liquid pharmaceutically administrable formulations can, for example, be prepared by dissolving, dispersing, or otherwise mixing the modified viral composition, e.g., the modified AAV composition, and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, Pa. Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intra-arterial infusion of a sterile aqueous solution containing a modified viral composition, e.g., modified AAV composition, described herein is an effective mode of administration. Another embodiment is a sterile aqueous or oily solution or suspension containing a modified viral composition, e.g., an modified AAV composition, described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a modified viral composition, e.g., a modified AAV composition, provided herein in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the modified viral compositions, e.g., modified AAV compositions s, provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

In specific embodiments, the a pharmaceutical composition described herein comprises 1×10⁶, 2×10⁶, 3×10⁶, 4×10⁶, 5×10⁶, 6×10⁶, 7×10⁶, 8×10⁶, 1×10⁶, 1×10⁷, 1×10⁶, 1×10⁷, 2×10⁷, 3×⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹ 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴ 9×10¹⁴ vector genomes (vg) of the modified viral composition, for example, the modified AAV composition.

### 5.5. Kits

In some embodiments, provided herein are kits comprising a modified viral composition described herein. In some embodiments, the kit further comprises instructions for administration of the modified viral composition. In some embodiments, the kit further comprises a solvent for the reconstitution of the viral composition.

In particular embodiments, provided herein are kits comprising a modified AAV composition described herein. In some embodiments, the kit further comprises instructions for administration of the modified AAV composition. In some embodiments, the kit further comprises a solvent for the reconstitution of the modified AAV composition.

In some embodiments, the kit comprises a bridging composition and/or a modified viral composition. In some embodiments, the kit further comprises instructions for administration of the compositions. In some embodiments, the kit further comprises a solvent for the reconstitution of the bridging composition and/or viral composition.

### 5.6. Gene Therapy Compositions

In certain aspects, a modified AAV composition, comprises an AAV particle described herein, that comprises a polynucleotide that comprises a transgene, e.g., any transgene described herein. Such a transgene may encode any polypeptide or polynucleotide sequence of interest. For example, the transgene may encode a sequence useful for gene therapy applications, e.g., may encode a sequence useful for gene replacement, gene silencing, gene addition or gene editing applications of gene therapy.

5.6.1. Transgenes

In certain embodiments, the transgene encodes a polypeptide, for example a biologically active copy of a protein, e.g., a protein useful for treating a disease or disorder. In specific embodiments, the transgene encodes two or more biologically active proteins. In other embodiments, the transgene encodes a detectable reporter protein, such as β-lactamase, β-galactosidase (LacZ), alkaline phosphatase, thymidine kinase, secreted alkaline phosphatase (SEAP), green fluorescent protein (GFP), chloramphenicol acetyltransferase (CAT), luciferase, membrane bound proteins including, for example, CD2, CD4, CD8, the influenza hemagglutinin protein, and others well known in the art. In some embodiments, the transgene is expressed in the target cell in the subject.

5.6.2. Gene Therapy Compositions

In certain embodiments, the transgene encodes a sequence useful for gene therapy applications. For example, certain diseases come about when one or more loss-of-function mutations within a gene reduce or abolish the amount or activity of the protein encoded by the gene. In certain embodiments, a transgene utilized herein encodes a functional, e.g., normal or wildtype, version of the protein.

In other embodiments, the transgene encodes a sequence useful for gene therapy applications that benefit from gene silencing. For example, certain diseases come about when gain-of-function mutations within a gene result in an aberrant amount or activity of the protein encoded by the gene. In certain embodiments, a transgene utilized herein encodes an inhibitory polynucleotide, e.g., an inhibitory RNA such as an miRNA or siRNA, or one or more components of gene editing system. e.g., a CRISPR gene editing system.

In other embodiments, the transgene encodes a sequence useful for gene therapy applications that benefit from gene addition. In certain embodiments, a transgene utilized herein encodes a gene product, e.g., a protein, not present in the recipient, e.g., the human subject, of the gene therapy.

In certain embodiments, the modified AAV compositions described herein comprise an AAV particle known in the art, e.g., an AAV particle that has been designed for use in gene therapy. In certain embodiments, for example, the modified AAV compositions described comprise an AAV particle of a gene therapy composition of Table 16. In certain embodiments, the modified AAV compositions described herein comprise an AAV particle that comprises a transgene listed in Table 16.

In Table 16, below, each row lists a gene therapy composition (right column), a transgene contained in the gene therapy composition (left column) and the disease or disorder (“conditions;” middle column) the gene therapy composition and transgene are associated with, that is, are designed to treat.

TABLE 16
Representative Gene Therapy Compositions

| Transgene | Conditions | Gene Therapy Composition |
|---|---|---|
| AADC (Aromatic L- | Aromatic L-amino Acid | PTC-AADC (PTC Therapeutics) |
| amino Acid | Decarboxylase (AADC) | AAV2-hAADC |
| Decarboxylase) | Deficiency | |
| | | VY-AADC01 (Voyager Therapeutics) |
| | Parkinson's Disease; Basal Ganglia | AAV-hAADC-2 |
| | Disease; Central Nervous System | |
| | Diseases; Movement Disorders; | |
| | Neurodegenerative Diseases; | |
| | Parkinsonian Disorders | |
| ABCD1 | Adrenoleukodystrophy | Lenti-D ™ (bluebird bio, Inc.) |
| ADA (Adenosine | severe combined immune | OTL-101 (Orchard Therapeutics), |
| deaminase) | deficiency due to adenosine | Strimvelis (Orchard Therapeutics) |
| | deaminase deficiency | |
| AAT (alpha-1 | Alpha 1-Antitrypsin Deficiency | rAAV2-CB-hAAT |
| antitrypsin) | | ADVM-043 (Adverum) |
| | | rAAV1-CB-hAAT |
| | | (Applied Genetic Technologies Corp) |
| AQP1 (aquaporin-1) | Squamous Cell Head and Neck | AAV2hAQP1 (MeiraGTx UK II Ltd) |
| | Cancer; Radiation Induced | |
| | Xerostomia; Salivary | |
| | Hypofunction | |
| ARSA (arylsulfatase A) | metachromatic leukodystrophy | OTL-200 (Orchard Therapeutics) |
| ARSB (arylsulfatase B) | Mucopolysaccharidosis Type VI | AAV2/8.TBG.hARSB |
| Ataxin-3 (silencing | Spinocerebellar ataxia type 3 | AMT-150 (Uniqure) |
| miRNA) | | |
| Beta globulin | beta-thalassemia major and severe | LentiGlobin ® BB305 (bluebird bio, Inc.) |
| | sickle cell disease | |
| Brain Derived | Glaucoma | QTA020V (Astellas) |
| Neurotrophic Factor | | |
| (BDNF) signaling | | |
| pathway | | |
| CFTR (cystic fibrosis | Cystic Fibrosis | AAV-CFTR |
| transmembrane | | |
| conductance regulator) | | |
| channelrhodopsin | Non-syndromic Retinitis | GS030-DP (GenSight Biologies) |
| ChrimsonR-tdTomato | Pigmentosa | |
| CLN2 | Late Infantile Neuronal Ceroid | AAV2CUhCLN2 |
| (ceroid lipofuscinosis, | Lipofuscinosis; Batten Disease | AAVrh.10CUCLN2 |
| neuronal, 2) | | |
| CLN3 (ceroid | CLN3; Batten Disease | AT-GTX-502 (Amicus Therapeutics) |
| lipofuscinosis, | | |
| neuronal, 3) | | |
| CLN6 (ceroid | Variant Late-Infantile Neuronal | AT-GTX-501 (Amicus Therapeutics) |
| lipofuscinosis, | Ceroid Lipofuscinosis | |
| neuronal, 6) | | |
| CNGA3 | Achromatopsia | AAV-CNGA3 (MeiraGTx UK II Ltd |
| (cyclic nucleotide gated | | AGTC-402 (Applied Genetic Technologies |
| channel subunit alpha 3) | | Corp) |
| CNGB3 | Achromatopsia | AAV-CNGB3 (MeiraGTx UK II Ltd) |
| (cyclic nucleotide gated | | rAAV2tYF-PR1.7-hCNGB3 (Applied |
| channel subunit beta 3) | | Genetic Technologies Corp) |
| Colagen C7 | Recessive Dystrophic | EB-101 (Abeona Therapeutics) |
| | Epidermolysis Bullosa | |
| Cytosine Deaminase | Cancer, high grade glioma | Toca 511 and Toca FC (Tocagen) |
| Dysferlin | Dysferlinopathy | rAAVrh74.MHCK7.DYSF.DV |
| Dystrophin | Duchenne Muscular Dystrophy | rAAVrh74.MHCK7.micro-dystrophin |
| | | (Sarepta Therapeutics, Inc.) |
| | | SGT-001 (Solid Biosciences, LLC); |
| | | rAAV2.5-CMV-minidystrophin (d3990, |
| | | Asklepios Biopharmaceutical, Inc.); |
| | | PF-06939926 (Pfizer), AT702 AT751 and |
| | | AT753 (all Audentes) |
| Factor IX | Hemophilia B | AAV2-hFIX16 (Spark Therapeutics) |
| | | AAV5-hFIXco-Padua (AMT-061) |
| | | (UniQure Biopharma B.V.) |
| | | AAV8-hFIX19 (Spark Therapeutics) |
| | | AAV with Human Factor IX (Avigen) |
| | | AskBio009 (Baxalta, now part of Shire) |
| | | BBM-H901 |
| | | DTX101 (Ultragenyx Pharmaceutical Inc) |
| | | FLT180a (Freeline) |
| | | SB-FIX (Sangamo Therapeutics) |
| | | scAAV2/8-LPl-hFIXco |
| | | SHP648 (Baxalta, now part of Shire) |
| | | SPK-9001 (Pfizer) |
| Factor VIII | Hemophilia A | SPK-8011, SPK-8001 (Spark |
| | | Therapeutics) |
| | | AMT-180 (Uniqure) |
| | | AAV2/8-HLP-FVIII-V3 |
| | | Recombinant AAV2/6 Human Factor VIII |
| | | Gene Therapy (Pfizer) |
| | | BMN 270 (BioMarin Pharmaceutical) |
| | | Valoctocogene Roxaparvovec (BioMarin |
| | | Pharmaceutical) |
| Factor VIII | Hemophilia A | BAX 888 (Baxalta now part of Shire) |
| (B-domain deleted) | | BAY2599023 (DTX201) (Bayer, |
| | | Ultragenyx Pharmaceutical Inc) |
| | | SB-525 (Pfizer) |
| Fit-1 | Macular Degeneration; Age- | rAAV.sFlt-1 (Adverum Biotechnologies, |
| (Fms related receptor | related Maculopathies; Retinal | Inc.) |
| tyrosine kinase 1) | Degeneration; Retinal | |
| | Neovascularization; Eye Diseases | |
| follistatin (FS344) | Becker Muscular Dystrophy; | rAAV1.CMV.huFollistatin344 (Milo |
| | Sporadic Inclusion Body Myositis | Therapeutics) |
| | Duchenne Muscular Dystrophy | |
| G11778G ND4 | Leber's Hereditary Optic | scAAV2-PIND4v2 |
| | Neuropathy | |
| G6Pase (Glucose-6- | GSD1 | DTX401 |
| Phosphatase) | Glycogen Storage Disease Type | (Ultragenyx Pharmaceutical Inc.) |
| | IA; Von Gierke's Disease | |
| GAA (acid alpha- | Pompe Disease | AT845 (Audentes Therapeutics): |
| glucosidase) | | rAAV1-CMV-GAA; |
| 6. | | Recombinant Adeno-Associated Virus |
| | | Acid Alpha-Glucosidase (Lacerta |
| | | Therapeutics, Inc); ATB200/AT2221 |
| | | (Amicus Therapeutics) |
| GAA (novel secretable | Pompe Disease: Glycogen Storage | SPK-3006 (Spark Therapeutics) |
| transgene) | Disease Type 2; Lysosomal | |
| | Storage Diseases; Acid Maltase | |
| | Deficiency | |
| GAD (Glutamate | Parkinson's Disease | rAAV-GAD (Neurologix, Inc.) |
| Decarboxylase) | | |
| gamma-sarcoglycan | Limb Girdle Muscular Dystrophy | AAV1-gamma-sarcoglycan (Genethon) |
| | Type 2C; Gamma- | |
| | sarcoglycanopathy | |
| GBA1 | Parkinson Disease | PR001A (Prevail Therapeutics) |
| (glucosylceramidase | | |
| beta) | | |
| GDNF (Glial cell line- | Parkinson's Disease | AAV2-GDNF (Brain Neurotherapy Bio, |
| derived neurotrophic | | Inc.) |
| factor) | | Convection enhanced delivery/AAV2- |
| | | GDNF |
| GLA (galactosidase | Fabry Disease; Lysosomal Storage | FLT190 (Freeline Therapeutics); ST-920 |
| alpha) | Diseases | (Sangamo) |
| | | AMT-190 (Uniqure) |
| GLB1 (galactosidase | Lysosomal Diseases; | AAV9-GLB1 (Axovant Sciences, Inc.) |
| beta 1) | Gangliosidosis\|GM1 | |
| | GM1 Gangliosidosis | LYS-GM101 (LYSOGENE) |
| GRN (granulin | Frontotemporal Dementia | PR006 (Prevail Therapeutics) |
| precursor) | | |
| CHM (Rab Escort | Choroideremia; CHM | AAV2-hCHM (SPK-7001, Spark |
| Protein) | (Choroideremia) Gene Mutations | Therapeutics); NSR-REP1 (NightstaRx |
| | | Ltd, a Biogen Company) |
| IFN-B (interferon beta) | Arthritis, Rheumatoid; Osteo | ART-I02 (Arthrogen) |
| | Arthritis | |
| apolipoprotein E2 | Alzheimer Disease; Early Onset | AAVrh.10hPOE2 |
| (APOE2) | Alzheimer Disease | |
| Human Lipoprotein | Familial Lipoprotein Lipase | Alipogene Tiparvovec (AMT-011, |
| Lipase [S447X] | Deficiency | Glybera ®) (Uniqure) |
| MTM1 (myotubularin) | X-Linked Myotubular Myopathy | AT132 (Audentes Therapeutics) |
| hαSG (human alpha- | Muscular Dystrophies | rAAV1.tMCK.human-alpha-sarcoglycan |
| sarcoglycan) | | |
| IDS | Mucopolysaccharidosis II; MPS II | SB-913 (Sangamo Therapeutics), RGX- |
| | | 121 (Regenxbio, Inc.) |
| IDUA (α-L-iduronidase) | MPS I | SB-318 (Sangamo Therapeutics), RGX- |
| | | 111 (Regenxbio, Inc.) |
| LAMP2B (lysosome- | Danon Disease | RP-A501 (Rocket Pharmaceuticals Inc.) |
| associated membrane | | |
| protein 2 isoform B) | | |
| LDLR (low density | Homozygous Familial | AAV directed hLDLR gene therapy |
| lipoprotein receptor) | Hypercholesterolemia (HoFH) | (Regenxbio Inc.) |
| MERTK (Mer tyrosine | Retinal Disease | rAAV2-VMD2-hMERTK |
| kinase) | | |
| NAGLU (N-acctyl- | Sanfilippo Syndrome B | rAAV2/5-hNAGLU (UniQure Biopharma |
| alpha-glucosaminidase) | | B.V.) |
| ND4 (NADH | Leber Hereditary Optic | GS010 (GenSight Biologics) |
| dehydrogenase 4 | Neuropathy | |
| Neurturin | Parkinson's Disease | CERE-120: Adeno-Associated Virus |
| | | Delivery of Neurturin (Sangamo |
| | | Therapeutics (Ceregene )) |
| NGF (nerve growth | Alzheimer's Disease | CERE-110: Adeno-Associated Virus |
| factor | | Delivery of NGF (Sangamo Therapeutics |
| | | (Ceregene )) |
| NTF3 | Charcot-Marie-Tooth Neuropathy | scAAV1.tMCK.NTF3 |
| (neurotrophin 3) | Type 1A | |
| OTC (Ornithine | Ornithine Transcarbamylase | DTX301 (Ultragenyx Pharmaceutical Inc) |
| Transcarbamylase) | (OTC) Deficiency | |
| p53 | Head and neck cancer | Gendicine (Shenzhen SiBiono GeneTech |
| | | Co. Ltd.) |
| PBGD | Acute Intermittent Porphyria | AAV2/5-PBGD (Digna Biotech S.L.) |
| (hydroxymethylbilane | | |
| synthase) | | |
| PDE6B | Retinitis Pigmentosa | AAV2/5-hPDE6B (Horama S.A.) |
| (phosphodiesterase 6B) | | |
| REP1 Rab Escort | Choroideremia | rAAV2.REP1 |
| Protein-1 (REP1) | | AAV-mediated REP1 gene replacement |
| | | AAV2-REP1 (NightstaRx Ltd. a Biogen |
| | | Company; Genzyme, a Sanofi Company) |
| REP65 (Retinal pigment | Leber Congenital Amaurosis | AAV OPTIRPE65 (MeiraGTx UK II Ltd) |
| epithelium-specific 65) | (LCA); Eye Diseases; Hereditary | |
| | Eye Diseases; Retinal Diseases | |
| | Leber Congenital Amaurosis | AAV RPE65 (MeiraGTx UK II Ltd) |
| | Inherited Retinal Dystrophy Due to | AAV2-hRPE65v2 (voretigene neparvovec- |
| | RPE65 Mutations; Leber | rzyl, Luxturna ®) |
| | Congenital Amaurosis | (Spark Therapeutics) |
| | Retinal Degeneration | rAAV2-CB-hRPE65 (Applied Genetic |
| | | Technologies Corp) |
| | | tgAAG76 (rAAV 2/2.hRPE65p.hRPE65) |
| | | (Targeted Genetics Corporation) |
| RPGR (Retinitis | X-Linked Retinitis Pigmentosa | AAV2/5-RPGR (MeiraGTx UK II Ltd) |
| Pigmentosa GTPase | | rAAV2YF-GRK1-RPGR (Applied |
| Regulator) | | Genetic Technologies Corp) |
| | | AAV-RPGR (MeiraGTx UK II Ltd) |
| | | AAV8-RPGR (NightstaRx Ltd, a Biogen |
| | | Company) |
| RS1 (retinoschisin 1 | X-linked Retinoschisis | rAAV2tYF-CB-hRS1 (Applied Genetic |
| polypeptide) | | Technologies Corp) |
| | Retinoschisis; X-Linked | RS1 AAV |
| SERCA2a (sarcoplasmic | Heart Failure, Congestive; Dilated | MYDICAR (SERCAZa gene) (Celladon |
| reticulum calcium | Cardiomyopathy | Corporation) |
| ATPase) | Ischemic Cardiomyopathy; Non- | AAV1/SERCA2a |
| | ischemic Cardiomyopathy; Heart | |
| | Failure; | |
| | Chronic Heart Failure; Patients | |
| | That Have Received a Left | |
| | Ventricular Assist Device | |
| SGSH (N- | Mucopolysaccharidosis III-A; | LYS-SAF302 (LYSOGENE) |
| sulfoglucosamine | MPS IIIA; Sanfilippo Syndrome; | ABO-102 (Abcona Therapeutics, Inc) |
| sulfohydrolase) | Sanfilippo A; | |
| SGSH and SUMF1 | Mucopolysaccharidosis Type III | SAF-301 (LYSOGENE) |
| | A; Sanfilippo Disease Type A | |
| | Spinal Muscular Atrophy | AVXS-101 (AveXis, Inc.) |
| SMN (survival motor | | onasemnogene abeparvovec-xioi |
| neuron) | | (Zolgensma ®) (AveXis, Inc., a Novartis |
| | | company) |
| telomerase (hTERT) | Aging | AAV-hTERT (Libella Gene Therapeutics) |
| | Critical Limb Ischemia | |
| | Alzheimer Disease | |
| TNFR:Fc (human tumor | Rheumatoid Arthritis; Psoriatic | tgAAC94 (Targeted Genetics Corporation) |
| necrosis factor receptor | Arthritis; Ankylosing Spondylitis | |
| (TNFR)- | | |
| immunoglobulin (IgG1) | | |
| Fc fusion gene) | | |
| UGT1A1 (UDP | Crigler-Najjar Syndrome | AT342 (Audentes Therapeutics) |
| glucuronosyltransferase | | GNT0003 (Genethon) |
| family 1 member A1) | | |
| α-Gal A (galactosidase | Fabry Disease | ST-920 (Sangamo Therapeutics) |
| alpha) | | |
| small nuclear RNA | Duchenne Muscular Dystrophy | scAAV9.U7.ACCA (Audentes |
| (saRNA) construct | | Therapeutics) |
| which induces exon | | |
| skipping | | |
| soluble decoy receptor | Wet Age-related Macular | ADVM-022 (Adverum Biotechnologies, |
| that binds vascular | Degeneration; Neovascular Age- | Inc.) |
| endothelial growth | related Macular Degeneration | |
| factor-A (VEGF-A), | | |
| VEGF-B and placental | | |
| growth factor (PIGF) | | |
| Suppressor of Factor | Blood Coagulation Disorders; | SPK-8016 (Spark Therapeutics) |
| VIII inhibitors | Coagulation Protein Disorders; | |
| | Factor VIII (FVIII) Deficiency; | |
| | Hematologic Diseases; | |
| | Hemorrhagic Disorders | |
| a soluble anti-VEGF | Neovascular Age-related Macular | RGX-314 (Regenxbio Inc.) |
| protein | Degeneration; Wet Age-related | |
| | Macular Degeneration | |
| genetically modified | melanoma | talimogene laherparepvec (Imlygic ™) |
| herpes simplex virus | | (Amgen) |
| type 1 designed to | | |
| replicate within tumors | | |
| and produce an | | |
| immunostimulatory | | |
| protein | | |
| Anti HCV shRNA | Hepatitis C | TT-034 (Tacere Therapeutics, Inc.) |
| HTT (huntingtin) | Hunting ton Disease | rAAV5-miHTT (AMT-130, UniQure |
| miRNA | | Biopharma B.V.) |
| VRC07 HIV-1 | HIV-1 Infected Adults With | VRC-HIVAAV070-00-GT (AAV8- |
| Neutralizing Antibody | Controlled Viremia | VRC07) |
| Wiskott-Aldrich | Wiskott-Aldrich Syndrome | OTL-103 (Orchard Therapeutics) |
| Syndrome (W AS) | | |

### 6.1. Modified Viral Compositions for Neutralizing Antibody Reduction

As summarized above, aspects of this disclosure include modified viral compositions that are useful for reducing levels or titers of neutralizing autoantibodies in a subject in need of viral therapy, e.g., gene therapy. In some embodiments, the modified viral composition includes empty viral particles that can bind to and internalize autoantibodies that can neutralize a target viral particle. Thus, aspects of this disclosure include a modified viral composition that has empty decoy viral particles of a target viral particle serotype.

It is understood that any embodiments of the modified viral compositions described herein can include be used in the methods of reducing levels or titers of neutralizing autoantibodies in a subject, e.g., a subject in need of viral therapy.

### 6.2. Methods of Use

6.2.1. Methods of Viral Transduction

As described above, upon binding of the cell surface receptor ligand or binding moiety to a target receptor present on a target cell, the modified viral composition is internalized into the cell. In some embodiments, the modified viral composition includes a transgene for delivery to the cell.

The term “transduction” as used herein in the context of a virus, for example AAV, refers to transfer of a virus particle, for example an AAV particle, whether alone or fused or conjugated or specifically bound to another molecule or molecules, into a cell.

Accordingly, provided herein are methods of viral transduction, comprising contacting a cell with a modified viral composition, e.g., as described herein, that comprises a virus particle, such that the modified viral composition enters the cell. In general, the efficiency of transduction of the modified viral composition into the cell is greater than that of an unmodified virus particle composition. In some embodiments of the method, the modified viral composition is a modified AAV composition (e.g., as described herein).

In certain aspects, presented herein are methods of viral transduction, comprising administering to a subject a pharmaceutical composition comprising a modified viral composition described herein, wherein the modified viral composition enters a target cell in the subject. In some embodiments of the method, the modified viral composition is a modified AAV composition (e.g., as described herein).

In certain embodiments, the subject is a human. In certain embodiments, the modified viral composition utilized comprises a virus particle, e.g., an AAV particle, which comprises a transgene. In particular embodiments, the modified viral composition utilized comprises an AAV particle which comprises a transgene useful for gene therapy. In particular embodiments, the modified viral composition utilized comprises an AAV particle which comprises a transgene useful for gene therapy, the subject is a human in need of the gene therapy, and the method of viral transduction is for use in treating the subject with the gene therapy.

In certain aspects, the method of viral transduction, e.g., AAV transduction, comprises contacting a cell with a modified viral composition, e.g., a modified AAV composition as described herein at a multiplicity of infection (MOI) of 30,000-100,000, wherein the modified viral composition, e.g., the modified AAV composition, enters the cells.

In certain embodiments, the cell comprises a cell surface mannose-6-phosphate receptor (M6PR), e.g., a cation-independent M6PR (CI-M6PR). In certain embodiments, the cell comprises a cell surface asialoglycoprotein receptor (ASGPR). In certain embodiments, the cell comprises a cell surface folate receptor, e.g., folate receptor 1 (FRα), or folate receptor 2 (FRβ) cell surface receptor.

In certain embodiments, a modified viral composition provided herein, e.g., a modified AAV composition provided herein, exhibits a higher transduction efficiency than the virus particle, e.g., the AAV particle, contained therein alone.

In some embodiments, the target cell is resistant to transduction by a particular virus (is a “virus transduction-resistant cell,” a “virus particle transduction-resistant cell” or a “viral particle transduction-resistant cell”). A cell is generally considered to be resistant to a particular virus if the cell is not transduced by the virus under normal transduction conditions, such conditions being well known to those of skill in the art, or if the cell is transduced by the virus under such conditions, but at substantially reduced levels, e.g., at less than 5%, less than 10%, less than 20%, less than 30%, less than 40%, or less than 50% of transduction levels observed under such conditions when using a reference cell known to be susceptible to transduction by the virus. In some embodiments, a cell may be resistant to transduction by a particular virus due to the presence of neutralizing antibodies (“NAbs”).

For example, in some embodiments, the cell is an AAV transduction-resistant cell (e.g., a Jurkat cell). An AAV transduction-resistant cell may be transduction-resistant for all AAV serotypes, a subset of AAV serotypes or one AAV serotype. A cell is generally considered to be AAV-resistant to a particular AAV, e.g., a particular AAV serotype, if the cell is not transduced by the AAV under normal transduction conditions, such conditions being well known to those of skill in the art, or if the cell is transduced by the AAV under such conditions, but at substantially reduced levels, e.g., at less than 5%, less than 10%, less than 20%, less than 30%, less than 40%, or less than 50% of transduction levels observed under such conditions when using a reference cell known to be susceptible to transduction by the AAV. In some embodiments, a cell may be resistant to transduction by one or more AAV serotypes virus due to the presence of NAbs.

In some embodiments, the transduced cell is a mammalian cell. In some embodiments, the cell is a muscle cell, neural cell, liver cell, cardiac cell, lung cell, immune cell, or kidney cell.

In some embodiments, a virus particle alone does not exhibit tropism for a cell. A given virus exhibits tropism for a cell when, under normal in vivo or in vitro conditions well known to those of skill in the art, the virus transduces the cell, for example, transduces the cell with a particular efficiency and/or transduces the cell with a particular level of preference relative to another cell. Generally, a given virus exhibits tropism for a particular set of cells, cell types and/or tissues. A virus's tropism for a cell, cell type or tissue may be assessed qualitatively or quantitatively.

In certain embodiments, a virus does not exhibit tropism for a cell, cell type or tissue if that virus does not normally transduce the cell, cell type, or cells or cells types of the tissue. In certain embodiments, a virus does not exhibit tropism for a cell, cell type or tissue if that virus transduces the cell, cell type, or cells or cells types of the tissue under normal in vivo or in vitro conditions, but does so at a substantially reduced level e.g., at less than 5%, less than 10%, less than 20%, less than 30%, less than 40%, or less than 50% of transduction levels the virus exhibits under such conditions when using a reference cell, cell type or tissue the virus is known to exhibit tropism for.

In some embodiments, the AAV particle alone does not exhibit tropism for the cell. A given AAV exhibits tropism for a cell when, under normal in vivo or in vitro conditions well known to those of skill in the art the AAV transduces the cell, for example, transduces the cell with a particular efficiency and/or transduces the cell with a particular level of preference relative to another cell. Generally, a given AAV serotype exhibits tropism for a particular set of cells, cell types and/or tissues. An AAV's tropism for a cell, cell type or tissue may be assessed qualitatively or quantitatively.

In certain embodiments, an AAV particle does not exhibit tropism for a cell, cell type or tissue if that AAV particles does not normally transduce the cell, cell type, or cells or cells types of the tissue. In certain embodiments, an AAV particle does not exhibit tropism for a cell, cell type or tissue if that AAV particles transduces the cell, cell type, or cells or cells types of the tissue under normal in vivo or in vitro conditions, but does so at a substantially reduced level e.g., at less than 5%, less than 10%, less than 20%, less than 30%, less than 40%, or less than 50% of transduction levels the AAV particle exhibits under such conditions when using a reference cell, cell type or tissue the AAV capsid is known to exhibit tropism for.

In certain embodiments, a modified viral composition presented herein exhibits a modified tropism relative to a viral composition, e.g., a viral particle, contained therein alone. For example, in certain embodiments, a modified viral composition presented herein exhibits a tropism for a cell, cell type or tissue that is at least 5%, 10%, 20%, 30%, 40%, 50%, 60%, 70%, 80%, 90%, 100%, 1.5-fold, 2-fold, 5-fold, 10-fold, 25-fold, 50-fold, 100-fold, 500-fold, or 1000-fold greater than that of the viral composition, e.g., the viral particle alone, wherein tropism measured by transduction into the cell, cell type, or tissue under normal in vitro or in vivo conditions.

In particular embodiments, such a modified viral composition maintains a tropism exhibited by the viral composition, e.g., viral particle, contained therein alone. For example, in certain embodiments, a modified viral composition maintains tropism for a cell, cell type or tissue exhibited by the viral composition, e.g., viral particle, alone, while also exhibiting a modified tropism as described above. In particular embodiments, a tropism exhibited by a viral composition, e.g., viral particle, alone is reduced or abolished when the viral composition, e.g., viral particle, is contained within such a modified viral composition. For example, in certain embodiments, a tropism exhibited by a viral composition, e.g., viral particle, alone is reduced or abolished when the viral composition, e.g., viral particle, is contained within such a modified viral composition, while the modified viral composition also exhibits a modified tropism as described above.

In certain embodiments, a modified AAV composition presented herein exhibits a modified tropism relative to an AAV composition, e.g., an AAV particle, contained therein. For example, in certain embodiments, a modified AAV composition presented herein exhibits a tropism for a cell, cell type or tissue that is at least 5%, 10%, 20%, 30%, 40%, 50%, 60%, 70%, 80%, 90%, 100%, 1.5-fold, 2-fold, 5-fold, 10-fold, 25-fold, 50-fold, 100-fold, 500-fold, or 1000-fold greater than that of the AAV composition, e.g., the AAV particle alone, wherein tropism measured by transduction into the cell, cell type, or tissue under normal in vitro or in vivo conditions.

In particular embodiments, such a modified AAV composition maintains a tropism exhibited by the AAV composition, e.g., AAV particle, contained therein alone. For example, in certain embodiments, a modified AAV composition maintains tropism for a cell, cell type or tissue exhibited by the AAV composition, e.g., AAV particle, alone, while also exhibiting a modified tropism as described above. In particular embodiments, a tropism exhibited by an AAV composition, e.g., AAV particle, alone is reduced or abolished when the viral composition, e.g., AAV particle, is contained within such a modified AAV composition. For example, in certain embodiments, a tropism exhibited by an AAV composition, e.g., AAV particle, alone is reduced or abolished when the AAV composition, e.g., AAV particle, is contained within such a modified AAV composition, while the modified AAV composition also exhibits a modified tropism as described above.

In some embodiments, the modified viral composition comprises a modified viral composition, e.g., a modified AAV composition provided herein that increases the transduction efficiency of a viral particle, e.g., an AAV particle, into a cell by at least 5% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 10% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 15% relative to the viral particle. e.g., the AAV particle, alone. In some embodiments, the modified viral composition, e.g., modified AAV composition, provided herein increases the transduction efficiency of a viral particle, e.g., an AAV particle, into a cell by at least 20% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 25% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 30% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 35% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 400 relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 45% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 50% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 55% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 60% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 65% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 70% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 75% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 80% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 85% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 90% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 95% relative to the viral particle, e.g., the AAV particle, alone. In some embodiments, the modified viral composition, e.g., modified AAV composition, provided herein increases the transduction efficiency of a viral particle, e.g., an AAV particle, into a cell by at least 2-fold, 3-fold, 4-fold, 5-fold, 6-fold, 7-fold, 8-fold, 9-fold, 10-fold, or more, relative to the AAV viral particle, e.g., the AAV particle, alone.

Methods of evaluating viral transduction, e.g., AAV transduction, are well known in the art. Representative methods are presented in the Examples, below. Successful viral transduction, e.g., AAV transduction, may be indicated, e.g., by expression of a transgene contained in a polynucleotide in the virus, e.g., AAV particle, in the cell into which the virus, e.g., AAV, is transduced. For example, expression of a transgene in a cell may be determined by measuring the expression level of the protein encoded by the transgene by, e.g., Western Blot, ELISA, immunofluorescence or tissue staining. Transgene expression in a cell may also be determined by measuring the presence of the RNA transcribed from the transgene, e.g., by RNA sequencing, real-time PCR or Northern Blot.

In some embodiments, the transgene is expressed at a sufficient level to be disease-modifying. Successful viral transduction, e.g., AAV transduction, may also be indicated by a minimal immune response to the virus, e.g., the AAV. A minimal immune response may be, e.g., an immune response that does not interfere with duration of transgene expression or with future re-administration of viral, e.g., AAV, therapy. In some embodiments, successful viral, e.g., AAV, transduction is indicated by expression of a transgene comprised by the viral, e.g., AAV, particle in a target tissue with limited toxicity. Limited toxicity is a level of toxicity that is acceptable in view of the benefits of successful transgene expression (e.g., expression of a transgene that is disease-modifying).

6.2.2. Methods of Reducing Neutralizing Antibody Titers

Many therapies employ viral therapy compositions, for example, viral therapy compositions for delivery of transgene of interest to a target cell. For example, many gene therapy compositions employ viral vectors such as AAV virus vectors to deliver a transgene of interest to a target cell. Since to differing degrees viruses are immunogenic, subjects undergoing viral therapy, such as gene therapy, who have already been exposed to the virus which is used in the particular viral therapy composition which the subject is administered, may have pre-existing immunity, which can include neutralizing antibodies (NAbs) to the viral therapy, e.g., gene therapy, composition. Viral therapy compositions can also lose efficacy with repeat dosing, due to NAbs generation.

NAbs are a very common issue in gene therapy using AAV (such as those gene therapy compositions set forth in Table 16), and have also been reported for other viral compositions that do not include AAV. For example, NAbs against viral therapy, e.g., gene therapy compositions comprising Newcastle Disease Virus or Herpes Simplex Virus (see Tayeb et al., Oncolytic Virotherapy 2015; 4 49-6; and Liu et al., Drug Delivery 2018, VOL. 25, NO. 01, 1950-1962, respectively) have been reported.

With respect to AAV, while AAV is generally considered to exhibit low immunogenicity, the virus still elicits an immune response, which immune response may, for example, be sufficient to reduce efficacy of AAV-based treatments, e.g., AAV-based gene therapies, or exclude a subject from being eligible for AAV-based gene therapy treatment. The immune response to AAV is known to vary between individuals, and by serotype. See, e.g., Louis Jeune et al. Hum Gene Ther Methods. 2013; 24(2):59-67. The modified viral compositions, e.g., modified AAV compositions, described herein may be utilized to reduce NAb titers (e.g., AAV NAb titers) in a subject in need thereof, e.g., a subject in need of viral treatment, for example, gene therapy treatment, such as AAV-based gene therapy treatment.

In certain aspects, presented herein is a method of reducing NAb titer (e.g., AAV NAb titer) in a subject in need thereof, comprising administering an amount of an modified viral compositions, e.g., a modified AAV composition, described herein to reduce NAb titer (e.g., AAV NAb titer) in the subject. In a particular embodiment, the modified viral composition comprises a viral protein or a viral particle, e.g., an empty viral particle. In particular embodiments, the modified AAV composition comprises an AAV virus particle. In certain embodiments, the AAV virus particle is an empty AAV virus particle. In particular embodiments, the modified AAV composition comprises a fragment of an AAV virus particle that specifically binds an AAV NAb. In particular embodiments, the modified AAV composition comprises an AAV viral proteins, for example, a VP1, VP2 or VP3 protein.

For example, in certain embodiments, provided herein is a method of reducing NAb titer (e.g., AAV NAb titer) in a subject in need thereof, comprising administering to the subject an amount of a modified viral composition, e.g., a modified AAV composition presented herein, that is effective to do so, wherein the modified viral composition, e.g., the modified AAV composition, comprises a viral particle or protein. In specific embodiments, the modified viral composition is a modified AAV composition and comprises an AAV virus particle, e.g., an empty AAV virus particle.

In certain aspects, presented herein is a method of reducing NAb titer (e.g., AAV NAb titer) in a subject in need thereof, comprising administering an effective amount of a modified viral composition, e.g., a modified AAV composition presented herein to reduce NAb titer in the subject, wherein the modified viral composition, e.g., the modified AAV composition, comprises a viral protein or particle that specifically binds to NAb. In specific embodiments, the modified viral composition is a modified AAV composition presented herein that comprises an AAV capsid protein or a fragment of an AAV capsid protein that specifically binds an AAV NAb.

For example, in certain embodiments, provided herein is a method of reducing neutralizing antibody titer in a subject in need thereof, comprising administering to the subject an amount of a modified viral composition, e.g., a modified AAV composition presented herein that is effective to do so, wherein modified viral composition comprises a viral particle or protein, e.g., an AAV capsid protein.

In certain embodiments, a method of reducing NAb titer is performed in a subject in need of viral therapy that has not previously received viral therapy treatment, and the method is performed prior to the subject beginning the viral therapy treatment. In certain embodiments, a method of reducing NAb titer is performed in a subject that has undergone viral therapy treatment or is undergoing viral therapy treatment and the method of reducing NAb titer is performed prior to the resumption of viral therapy treatment (e.g., prior to the next dose in a viral therapy treatment regimen) in the subject. In certain embodiments, a method of reducing NAb titer is performed in the subject concurrently with the viral therapy treatment. In certain embodiments, a method of reducing NAb titer is performed in a subject that has previously undergone a viral therapy treatment and the method of reducing NAb titer is performed prior to the subject beginning another, e.g., different, viral therapy treatment.

In particular embodiments, a method of reducing NAb titer is performed in a subject in need of gene therapy that has not previously received gene therapy treatment, and the method is performed prior to the subject beginning the gene therapy treatment. In certain embodiments, a method of reducing NAb titer is performed in a subject that has undergone gene therapy treatment or is undergoing gene therapy treatment and the method of reducing NAb titer is performed prior to the resumption of gene therapy treatment (e.g., prior to the next dose in a gene therapy treatment regimen) in the subject. In certain embodiments, a method of reducing NAb titer is performed in the subject concurrently with the gene therapy treatment. In certain embodiments, a method of reducing NAb titer is performed in a subject that has previously undergone a gene therapy treatment and the method of reducing NAb titer is performed prior to the subject beginning another, e.g., different, gene therapy treatment.

This disclosure includes a method of reducing neutralizing antibody (Nab) titer in a subject in need thereof, comprising: administering any of the modified viral compositions of the preceding embodiments or a pharmaceutical composition comprising any of the modified viral compositions of the preceding embodiments to the subject, such that NAb titer in the subject is reduced. In some embodiments, the modified viral composition comprises an empty virus particle. In some embodiments, the modified viral composition comprises a viral protein. In some embodiments, the subject is a human in need of viral therapy, and wherein administering the modified viral composition is performed prior to the onset of the viral therapy. In some embodiments, administering the modified viral composition is performed 1 to 6 hours prior to the onset of the viral therapy. In some embodiments, the method further comprises administering the viral therapy to the subject following the administering of the modified viral composition.

In some embodiments of the method of reducing neutralizing antibody titer, the subject is a human undergoing viral therapy. In some embodiments, the modified viral composition is administered to the subject concurrently with the viral therapy. In some embodiments, the subject is a human who has previously undergone viral therapy and is in need of additional viral therapy. In some embodiments, administering the modified viral composition is performed 1 to 6 hours prior to onset of the additional viral therapy. In some embodiments, the method further comprises administering the additional viral therapy to the subject following administering the modified viral composition.

In another aspect, provided herein is a method of reducing AAV neutralizing antibody (Nab) titer in a subject in need thereof, comprising administering any of the modified viral compositions of the preceding embodiments or a pharmaceutical composition comprising any of the modified viral compositions of the preceding embodiments to the subject, wherein the viral composition comprises an AAV composition, such that NAb titer in the subject is reduced. In some embodiments, the modified viral composition comprises an empty AAV particle. In some embodiments, the modified viral composition comprises an AAV viral protein. In some embodiments, the AAV viral protein is an AAV VP1, VP2 or VP3 protein.

In some embodiments of the method of reducing AAV neutralizing antibody titer in a subject, the subject is a human in need of AAV-based gene therapy, and administering the modified viral composition comprising the AAV composition is performed prior to the onset of the gene therapy. In some embodiments, administering the modified viral composition comprising the AAV composition is performed 1 to 6 hours prior to the onset of the gene therapy. In some embodiments, the method further comprises administering the gene therapy to the subject following the administering of the modified viral composition. In some embodiments, the subject is a human undergoing AAV-based gene therapy. In some embodiments, the modified viral composition comprising the AAV composition is administered to the subject concurrently with the gene therapy.

In some embodiments of the method of reducing AAV neutralizing antibody titer in a subject, the subject is a human who has previously undergone gene therapy and is in need of additional AAV-based gene therapy. In some embodiments, administering the modified viral composition comprising the AAV composition is performed 1 to 6 hours prior to onset of the additional gene therapy. In some embodiments, the additional gene therapy to the subject following administering the modified viral composition. In some embodiments, the AAV of the AAV composition is an AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV11, AAV12, AAV13, AAV LK03, AAVrh74, AAV DJ, AAV Anc81, Anc82, Anc83, Anc84, Anc110, Anc113, Anc126, or Anc127, AAV hu·37, AAV rh·8, AAV_go·1, AAV LK03, or AAV rh74 serotype.

Also provided herein are methods of reducing neutralizing antibody titers in a subject, comprising administering to the subject an effective amount of a modified viral composition presented herein, wherein the modified viral composition comprises a bridging composition presented herein specifically bound to an AAV particle.

In certain embodiments, a method of reducing neutralizing antibodies is performed in conjunction with a method of treating a disease or disorder comprising a viral therapy. In a particular embodiment, a method of reducing neutralizing antibody titer as described herein may be used in conjunction with a method of treating a disease or disorder that comprises use of a modified viral composition presented herein. In other particular embodiments, a method of reducing neutralizing antibody titer as presented herein may be performed in conjunction with a method of treating a disease or disorder that comprises a viral treatment known in the art. For example, the method of reducing neutralizing antibody titers may be performed prior to the onset of the viral therapy or concurrently with the viral therapy, in a subject who has not previously received the viral therapy, is undergoing viral therapy, or who is in need of additional viral therapy. In particular embodiments, a method of reducing AAV neutralizing antibodies is performed in conjunction with a method of treating a disease or disorder comprising an AAV-based gene therapy. For example, the method of reducing AAV neutralizing antibody titers may be performed prior to the onset of the gene therapy or concurrently with the gene therapy, in a subject who has not previously received the gene therapy, is undergoing the gene therapy, or who is in need of additional AAV-based gene therapy. In certain embodiments, the gene therapy is AAV-based gene therapy.

In certain embodiments, the methods of reducing NAb titer described herein are utilized in conjunction with a method of treating a disease or disorder in a subject in need thereof, that comprises administering to the subject an effective amount of a gene therapy composition. In particular embodiments, the NAb titer is reduced using a modified viral composition presented herein, wherein the gene therapy composition comprises a viral vector that is not AAV. In specific embodiments, the gene therapy comprises a viral vector that is a lentivirus (e.g., HIV-1, HIV-2) a human herpes virus (e.g., HSV-1, HSV-2, varicella-zoster virus, Epstein-Barr virus, or cytomegalovirus), an adenovirus, a Newcastle Disease Virus, a vaccinia virus, or a vesicular stomatitis virus, for example, a modified viral composition a described herein that comprises such a viral vector. In other specific embodiments, the NAb titer is reduced using a modified viral composition presented herein, and the gene therapy composition may comprise a modified AAV composition described herein. In other specific embodiments, the NAb titer is reduced using a modified viral composition presented herein, wherein the gene therapy composition need not comprise an AAV composition described herein.

### 6.3. Methods of Treatment

Also provided herein are methods of treating a disease or disorder in a subject, comprising administering to the subject an effective amount of a modified viral composition presented herein, wherein the modified viral composition comprises a virus particle. In certain embodiments, provided herein are methods of treating a disease or disorder in a subject, comprising administering to the subject a pharmaceutical composition comprising an effective amount of a modified viral composition presented herein, wherein the modified viral composition comprises a virus particle.

In some embodiments, the transduction efficiency of a modified viral composition utilized in a method of treatment described herein is at least 10% greater than that of the viral particle contained therein alone. In some embodiments, the transduction efficiency of a modified viral composition utilized in a method of treatment described herein is at least 20% greater than that of the viral particle contained therein alone. In some embodiments, the transduction efficiency of a modified viral composition utilized in a method of treatment described herein is at least 30% greater than that of the viral particle contained therein alone. In some embodiments, the transduction efficiency of a modified viral composition utilized in a method of treatment described herein is at least 30% greater than that of the viral particle contained therein alone. In some embodiments, the transduction efficiency of a modified viral composition utilized in a method of treatment described herein is at least 40% greater than that of the viral particle contained therein alone. In some embodiments, the transduction efficiency of a modified viral composition utilized in a method of treatment described herein is at least 50% greater than that of the viral particle contained therein alone. In some embodiments, the transduction efficiency of a modified viral composition utilized in a method of treatment described herein is at least 60% greater than that of the viral particle contained therein alone. In some embodiments, the transduction efficiency of a modified viral composition utilized in a method of treatment described herein is at least 70% greater than that of the viral particle contained therein alone. In some embodiments, the transduction efficiency of a modified viral composition utilized in a method of treatment described herein is at least 80% greater than that of the viral particle contained therein alone. In some embodiments, the transduction efficiency of a modified viral composition utilized in a method of treatment described herein is at least 90% greater than that of the viral particle contained therein alone. In some embodiments, the transduction efficiency of a modified viral composition utilized in a method of treatment described herein is at least 2-fold, 3-fold, 4-fold, 5-fold, 6-fold, 7-fold, 8-fold, 9-fold, 10-fold or more, greater than that of the viral particle contained therein alone.

In some embodiments, the method of treatment provided herein utilizes a modified viral composition provided herein to deliver a transgene to a cell that is virus transduction-resistant. In specific embodiments, expression of a transgene of the modified viral composition in a target cell can be achieved by administering a lower dose of the modified viral composition than would be required of a viral composition comprising the viral particle alone, for example, a viral composition comprising a similar viral composition but no cell surface receptor binding moiety. In specific embodiments, transgene expression in a target cell can be achieved by administering a vector genome (vg) dose of the modified viral composition that is 10%, 20%, 30%, 40%, 50%, 60%, 70% 80% or 90% lower than the dose than would be required of a viral composition comprising the viral particle alone.

In some embodiments, a subject to be treated with a method provided herein has NAbs against a virus, e.g., a virus of the serotype as that used in a modified viral composition provided herein. In specific embodiments, the subject has NAb titers that exclude the subject from participation in the method of treatment with a viral composition comprising the viral particle alone against which the subject has NAbs. Titers of neutralizing antibodies may be expressed as units per volume (e.g., u/mL). Titers of neutralizing antibodies may be expressed as a dilution of a test sample at which dilution at which at least 50% inhibition of transgene expression is measured. Methods of determining NAb titers are well known in the art; see below for an exemplary method of measuring NAb titers. In certain embodiments, the subject has a NAb titer of about 1:2, 1:5, 1:10 or 1:400. In certain embodiments, the subject has a NAb titer of above 200 U/mL.

In certain embodiments, the disease or disorder treated in accordance with the methods described herein is cancer. In certain embodiments, the diseases treated in accordance with the methods described herein is a neurological disease. In certain embodiments, the diseases treated in accordance with the methods described herein is a neurodegenerative disease. In certain embodiments, the diseases treated in accordance with the methods described herein is a cardiovascular disorder. In certain embodiments, the diseases treated in accordance with the methods described herein is an ocular disease.

6.3.1. AAV Methods

In certain aspects, provided herein are methods of treating a disease or disorder in a subject, comprising administering to the subject an effective amount of a modified AAV composition presented herein, wherein the modified AAV composition an AAV particle. For example, in certain aspects, presented herein are methods of treating a disease or disorder by administering to a subject in need thereof a pharmaceutical composition comprising an effective amount of a modified AAV composition described herein.

In certain embodiments, an AAV particle utilized as part of a method of treatment described herein comprises a polynucleotide that comprises a transgene. Such a transgene may, for example, encode any polypeptide or polynucleotide sequence useful for treatments involving such applications of gene therapy as gene replacement, gene silencing, gene addition or gene editing.

In some embodiments, the transduction efficiency of a modified AAV composition utilized in a method of treatment described herein is at least 10% greater than that of the AAV particle contained therein alone. In some embodiments, the transduction efficiency of a modified AAV composition utilized in a method of treatment described herein is at least 20% greater than that of the AAV particle contained therein alone. In some embodiments, the transduction efficiency of a modified AAV composition utilized in a method of treatment described herein is at least 30% greater than that of the AAV particle contained therein alone. In some embodiments, the transduction efficiency of a modified AAV composition utilized in a method of treatment described herein is at least 30% greater than that of the AAV particle contained therein alone. In some embodiments, the transduction efficiency of a modified AAV composition utilized in a method of treatment described herein is at least 40% greater than that of the AAV particle contained therein alone. In some embodiments, the transduction efficiency of a modified AAV composition utilized in a method of treatment described herein is at least 50% greater than that of the AAV particle contained therein alone. In some embodiments, the transduction efficiency of a modified AAV composition utilized in a method of treatment described herein is at least 60% greater than that of the AAV particle contained therein alone. In some embodiments, the transduction efficiency of a modified AAV composition utilized in a method of treatment described herein is at least 70% greater than that of the AAV particle contained therein alone. In some embodiments, the transduction efficiency of a modified AAV composition utilized in a method of treatment described herein is at least 80% greater than that of the AAV particle contained therein alone. In some embodiments, the transduction efficiency of a modified AAV composition utilized in a method of treatment described herein is at least 90% greater than that of the AAV particle contained therein alone. In some embodiments, the transduction efficiency of a modified AAV composition utilized in a method of treatment described herein is at least 2-fold, 3-fold, 4-fold, 5-fold, 6-fold, 7-fold, 8-fold, 9-fold, 10-fold or more, greater than that of the AAV particle contained therein alone.

In some embodiments, the method of treatment provided herein utilizes a modified AAV composition provided herein to deliver a transgene to a cell that is AAV transduction-resistant. In specific embodiments, expression of transgene of the modified AAV composition in a target cell can be achieved by administering a lower dose of the modified AAV composition than would be required of an AAV composition, e.g., a gene therapy composition, comprising the AAV particle alone, for example, an AAV composition, e.g., a gene therapy composition comprising a similar AAV composition but no cell surface-binding moiety. In specific embodiments, transgene expression in a target cell can be achieved by administering a vector genome (vg) dose of the modified AAV composition that is 10%, 20%, 30%, 40%, 50%, 60%, 70% 80% or 90% lower than the dose than would be required of for an AAV composition, e.g., gene therapy composition, comprising the AAV particle alone.

In some embodiments, a subject to be treated with a method provided herein has NAbs against AAV. In specific embodiments, the subject has anti-AAV NAb titers that exclude the subject from participation in the method of treatment with an AAV composition comprising the AAV particle alone. Titers of neutralizing antibodies may be expressed as units per volume (e.g., u/mL). Titers of neutralizing antibodies may be expressed as a dilution of the test sample at which dilution at which at least 50% inhibition of transgene expression is measured. See below for an exemplary method of measuring NAb titers. In certain embodiments, the subject has an anti-AAV, e.g., anti-AAV2, AAV5, AAV8 or anti-AAV9, NAb titer of about 1:2, 1:5, 1:10 or 1:400. In certain embodiments, the subject has an anti-AAV, e.g., anti-AAV2, anti-AAV5, anti-AAV8 or anti-AAV9. NAb titer of above 200 U/mL.

Methods of determining NAb titers in a subject are well-known in the art. Generally, NAb titers are determined by exposing cells to increasing levels of a biological sample that contains the NAbs (e.g., plasma, serum, synovial fluid, cerebrospinal fluid, etc.) and determining transduction efficiency of a reporter vector (e.g., an AAV vector expressing a transgene encoding a detectable protein, such as GFP or luciferase). The neutralizing titer of a sample is then calculated by determining the first dilution at which at least 50% inhibition of transgene expression is measured. See Meliani et al., Hum Gene Ther Methods. 2015; 26:45-53 for an exemplary assay protocol.

Methods to detect pre-existing AAV immunity also include cell-based in vitro TI assays, in vivo (e.g., mice) TI assays, and enzyme-linked immunosorbent assay (ELISA)-based detection of total anti-capsid antibody (TAb) assays. (Masat et al., Discov Med 2013 15:379-389; Boutin et al., Hum Gene Ther 2010 21:704-712). The TAb assay may be able to detect low potency NAb that are below the threshold of T1 assays, but it may not detect non-antibody neutralizing factors. In vivo and in vitro TI assays screen samples for anti-AAV Nab (Manno et al., Nat Med 2006, 12:342-347, Boutin et al., Hum Gene Ther 2010; 21: 704-712, Calcedo et al. Clin Vaccine Immunol 2011; 18: 1586-1588, Mingozzi et al., Gene Ther 2013; 20: 417-424, Calcedo et al., J Infect Dis 2009: 199: 381-390, Halbert et al., Hum Gene Ther 2006; 17: 440-447, Li et al., Gene Ther 2012; 19: 288-294, Moskalenko et al., J Virol 2000; 74: 1761-1766, Wang et al. Mol Ther 2010; 18: 126-134, Grimm et al., J Virol 2008; 82: 5887-5911, Greenberg et al. Gene Ther 2016; 23: 313-319, Sun et al., J Immunol Methods 2013; 387: 114-120) and other factors that modulate AAV transduction efficiency (Berry et al. Mol Ther 2016: 24(Suppl 1): S14 (abstract 30), Weinberg et al. J Virol 2014; 88: 12472-12484, Hirosue et al. Virology 2007; 367: 10-18, Virella-Lowell et al., Gene Ther 2000: 7: 1783-1789, Mitchell et al. J Virol 2013: 87: 13035-13041, Mitchell et al., J Virol 2013; 87: 4571-4583, Berry et al. J Biol Chem 2016; 291: 939-947, Nonnenmacher et al. Gene Ther 2012; 19: 649-658). Similar methods may be employed to determine NAb titer against other viruses.

In certain embodiments, an AAV particle utilized as part of a method of treatment described herein comprises a polynucleotide that comprises a transgene useful for implementing gene replacement applications of gene therapies. In certain embodiments, a method of treatment described herein treats a disease or disorder that comes about when one or more loss-of-function mutations within a gene reduce or abolish the amount or activity of the protein encoded by the gene. In certain embodiments, a method of treatment described herein utilizes an AAV particle that comprises a transgene that encodes a functional, e.g., normal or wildtype, version of the protein. For example, in certain embodiments, a method of treatment described herein utilizes an AAV particle that comprises a transgene encoding a biologically active copy of a protein useful for treating such a disease or disorder, whereby the transgene expresses the polypeptide in a target cell of the subject in need of treatment. In certain embodiments, such a method of treatment utilizes an AAV particle that comprises a transgene described herein that encodes a biologically active form of a polypeptide described herein, and expresses the polypeptide in a target cell of the subject in need of treatment.

In particular embodiments, the subject treated in such a method of treatment is a human subject and the method utilizes an AAV particle that comprises a transgene that encodes a biologically active form of a human a polypeptide, and expresses the polypeptide in a target cell of the human subject in need of treatment.

In certain embodiments, an AAV particle utilized as part of a method of treatment described herein comprises a polynucleotide that comprises a transgene useful for implementing gene addition applications of gene therapies. In certain embodiments, a method of treatment described herein treats a disease or disorder by delivering to a subject in need thereof a transgene that encodes a gene product not present in the subject, and expresses the gene product, e.g., polypeptide, in a target cell of the subject. In certain embodiments, a method of treatment described herein utilizes an AAV particle that comprises a transgene encoding the gene product, e.g., protein, whereby the transgene expresses the gene product, e.g., protein, in a target cell of the subject in need of treatment In certain embodiments, such a method of treatment utilizes an AAV particle that comprises a transgene described herein that encodes a protein described herein, and expresses the protein in a target cell of the subject in need of treatment.

In particular embodiments, the subject treated in such a method of treatment is a human subject and the method utilizes an AAV particle that comprises a transgene that encodes a human a polypeptide, and expresses the polypeptide in a target cell of the human subject in need of treatment.

In certain embodiments, an AAV particle utilized as part of a method of treatment described herein comprises a polynucleotide that comprises a transgene useful for implementing gene silencing applications of gene therapies. In certain embodiments, a method of treatment described herein treats a disease or disorder that comes about when gain-of-function mutations within a gene result in an aberrant amount or activity of the protein encoded by the gene. In certain embodiments, a method of treatment described herein utilizes an AAV particle that comprises a transgene that encodes a polynucleotide, e.g., an RNA such as an inhibitory RNA, that inhibits the expression or activity of the gene or mRNA product(s) of the gene. In particular embodiments, the transgene encodes a micro RNA (miRNA) or a silencer RNA (siRNA). For example, in certain embodiments, a method of treatment described herein utilizes an AAV particle that comprises a transgene encoding an RNA that inhibits the expression or activity of the gene or mRNA product(s) of the gene, e.g., encodes an miRNA or an siRNA, useful for treating such a disease or disorder, whereby the transgene expresses the RNA in a target cell of the subject in need of treatment. In certain embodiments, such a method of treatment utilizes an AAV particle that comprises a transgene described herein that encodes an RNA, e.g., miRNA or siRNA, described herein, and expresses the RNA in a target cell of the subject in need of treatment.

In particular embodiments, the subject treated in such a method of treatment is a human subject and the method utilizes an AAV particle that comprises a transgene that expresses the RNA in a target cell of the human subject in need of treatment.

In yet other embodiments, an AAV particle utilized as part of a method of treatment described herein comprises a polynucleotide that comprises a transgene encoding gene editing system or a component of a gene editing system, e.g., a zinc-finger nuclease (ZFN), transcription activator-like effector nuclease (TALEN) or CRISPR gene editing system. The particular transgene or transgenes utilized as part of such methods may be designed, as necessary, to be useful for treatment of diseases and disorders caused by loss-of-function mutations or gain-of-function mutations, or for treatment of disorder that benefit from gene addition.

In certain embodiments, a method of treatment presented herein utilizes a modified AAV composition described herein that comprises an AAV particle comprising a transgene listed in Table. In specific embodiments, a method of treatment presented herein utilizes a modified AAV composition described herein, comprising a gene therapy composition listed in Table. In certain embodiments, a method of treatment presented herein utilizes an AAV composition described herein that comprises an AAV particle comprising a transgene listed in Table, wherein the transgene is expressed in a target cell of the subject in need of treatment. In certain embodiments, a method of treatment presented herein utilizes a modified AAV composition described herein that comprises a gene therapy composition listed in Table, wherein the transgene of the gene therapy composition is expressed in a target cell of the subject in need of treatment.

In certain embodiments, a method of treatment presented herein treats a disease or disorder listed in Table. In a particular embodiment, a method of treatment presented herein comprises administering to a subject in need of treatment of a disease or disorder listed in Table a pharmaceutical composition, wherein the pharmaceutical composition comprises an effective amount of a modified AAV composition described herein, wherein the AAV composition comprises an AAV particle, and wherein the AAV particle comprises a polynucleotide that comprises a transgene listed in Table as associated with the disease or disorder. In a specific embodiment of such a method, the transgene is expressed in a target cell of the subject. In a particular embodiment, a method of treatment presented herein comprises administering to a subject in need of treatment of a disease or disorder listed in Table a pharmaceutical composition, wherein the pharmaceutical composition that comprises an effective amount of a modified AAV composition described herein, wherein the AAV composition comprises a gene therapy composition listed in Table as associated with the disease or disorder. In a specific embodiment of such a method, the transgene of the gene therapy composition is expressed in a target cell of the subject.

In certain embodiments, a method of treatment presented herein utilizes a modified AAV composition described herein that comprises an AAV particle comprising a transgene encoding human Factor 1X. In specific embodiments, a method of treatment presented herein utilizes a modified AAV conjugate described herein, comprising AMT-061 or SPK-9001. In certain embodiments, a method of treatment presented herein utilizes a modified AAV composition described herein, that comprises an AAV particle comprising a transgene encoding human Factor IX, wherein the transgene is expressed in a target cell of the subject in need of treatment. In certain embodiments, a method of treatment presented herein utilizes a modified AAV composition described herein, that comprises AMT-061 or SPK-9001, wherein the transgene of the gene therapy composition is expressed in a target cell of the subject in need of treatment.

In certain embodiments, a method of treatment presented herein treats a hemophilia B. In a particular embodiment, a method of treatment presented herein comprises administering to a subject in need of treatment of hemophilia B a pharmaceutical composition, wherein the pharmaceutical composition comprises an effective amount of a modified AAV composition described herein, wherein the modified AAV composition comprises an AAV particle, and wherein the AAV particle comprises a polynucleotide that comprises a transgene encoding human Factor IX In a specific embodiment of such a method, the transgene is expressed in a target cell of the subject. In a particular embodiment, a method of treatment presented herein comprises administering to a subject in need of treatment of hemophilia B a pharmaceutical composition, wherein the pharmaceutical composition comprises an effective amount of a modified AAV composition described herein, wherein the modified AAV composition comprises AMT-061 or SPK-9001 as associated with the disease or disorder. In a specific embodiment of such a method, the Factor IX transgene of the gene therapy composition is expressed in a target cell of the subject.

In certain embodiments, a method of treatment presented herein utilizes a modified AAV composition described herein, that comprises an AAV particle comprising a transgene encoding RPE65. In specific embodiments, a method of treatment presented herein utilizes a modified AAV composition described herein comprising voretigene neparvovec-rzyl. In certain embodiments, a method of treatment presented herein utilizes a modified AAV composition described herein that comprises an AAV particle comprising a transgene encoding RPE65, wherein the transgene is expressed in a target cell of the subject in need of treatment. In certain embodiments, a method of treatment presented herein utilizes a modified AAV composition described herein, that comprises voretigene neparvovec-rzyl, wherein the transgene of the gene therapy composition is expressed in a target cell of the subject in need of treatment.

In certain embodiments, a method of treatment presented herein treats inherited retinal dystrophy. In a particular embodiment, a method of treatment presented herein comprises administering to a subject in need of inherent retinal dystrophy a pharmaceutical composition, wherein the pharmaceutical composition comprises an effective amount of a modified AAV composition described herein, wherein the modified AAV composition comprises an AAV particle, and wherein the AAV particle comprises a polynucleotide that comprises a transgene encoding RPE65 as associated with the disease or disorder. In a specific embodiment of such a method, the transgene is expressed in a target cell of the subject. In a particular embodiment, a method of treatment presented herein comprises administering to a subject in need of treatment of inherited retinal dystrophy a pharmaceutical composition, wherein the pharmaceutical composition comprises an effective amount of a modified AAV composition described herein, wherein the AAV composition comprises voretigene neparvovec-rzyl. In a specific embodiment of such a method, the RPE65 transgene of the gene therapy composition is expressed in a target cell of the subject.

### 6.4. Methods of Producing Virus and Viral Proteins

The viruses described herein may be produced using any suitable method known in the art. For example, a host cell (e.g., an insect or mammalian cell) may be engineered to stably expresses the necessary components for virus particle production. The use of a selectable marker allows for large-scale production of recombinant virus.

### 6.5. Methods of Producing AAV Particles and Capsid Proteins

The AAV particles described herein may be produced using any suitable method known in the art. For example, a host cell (e.g., an insect or mammalian cell) may be engineered to stably expresses the necessary components for AAV particle production. This can be achieved by integrating a plasmid (or multiple plasmids) comprising AAV rep and cap genes, and a selectable marker, such as an antibiotic (e.g., neomycin or ampicillin) resistance gene into the genome of the cell. The cell can be an insect or mammalian cell which can then be co-infected with a helper virus (e.g., adenovirus or baculovirus providing the helper functions) and the viral vector comprising the 5′ and 3′ AAV ITR. The use of a selectable marker allows for large-scale production of the recombinant AAV. As another non-limiting example, adenovirus or baculovirus rather than plasmids can be used to introduce rep and cap genes into packaging cells. As yet another non-limiting example, both the viral vector containing the 5′ and 3′ AAV LTRs and the rep and cap genes can be stably integrated into the DNA of producer cells, and the helper functions can be provided by a wild-type adenovirus to produce the recombinant AAV.

A “helper virus” for AAV refers to a virus that allows AAV to be replicated and packaged by a host cell. A helper virus provides helper functions which allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions.

A preparation of AAV is said to be “substantially free” of helper virus if the ratio of infectious AAV particles to infectious helper virus particles is at least about 102:1; at least about 104:1, at least about 106:1; or at least about 108 1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

A “helper virus” for AAV refers to a virus that allows AAV to be replicated and packaged by a host cell. A helper virus provides helper functions which allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions.

A preparation of AAV is said to be “substantially free” of helper virus if the ratio of infectious AAV particles to infectious helper virus particles is at least about 102:1; at least about 104:1, at least about 106:1; or at least about 108:1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

### 6.6. Polynucleotide Construction

AAV particles may comprise a polynucleotide, as discussed herein. Polynucleotides, used in the present disclosure can be constructed according to known techniques. For example, the polynucleotide, may be constructed to include operatively linked components as described herein. The regulatory sequences to be included can be selected based on the cell of interest.

In some embodiments, a polynucleotide comprising, e.g., a transgene and selected regulatory sequences flanked by AAV ITRs can be constructed by directly inserting the polynucleotide of interest into an AAV genome, e.g., into excised AAV open reading frames, and certain portions of the AAV genome can optionally be deleted, as described in, e.g., WO 1993/003769; Kotin (1994) Human Gene Therapy 5:793-801; Shelling and Smith (1994) Gene Therapy 1:165-169; and Zhou et al. (1994) J. Exp. Med. 179:1867-1875.

In other embodiments, AAV ITRs are excised from an AAV genome containing such ITRs, and then are fused to 5′ and 3′ of the polynucleotide sequence of interest that is present in another polynucleotide using standard ligation techniques.

In certain embodiments, the polynucleotide provided herein comprises a recombinant self-complementing genome. A polynucleotide comprising a self-complementing genome can usually quickly form a double stranded DNA molecule by its partially complementing sequences (e.g., complementing coding and non-coding strands of a transgene). More specifically, in some embodiments, an AAV vector provided herein comprises an AAV genome that comprises a first heterologous polynucleotide sequence (e.g., a therapeutic transgene coding strand) and a second heterologous polynucleotide sequence (e.g., the noncoding or antisense strand of the therapeutic transgene), and the first heterologous polynucleotide sequence can form intrastrand base pairs with the second polynucleotide sequence. In some embodiments, the first heterologous polynucleotide sequence and a second heterologous polynucleotide sequence are linked by a sequence that facilitates intrastrand basepairing, e.g., a hairpin DNA structure. In some embodiments, the first heterologous polynucleotide sequence and a second heterologous polynucleotide sequence are linked by a mutated ITR, so that the rep proteins do not cleave the viral genome at the mutated ITR. In a specific embodiment, a recombinant viral genome comprises the following in 5′ to 3′ order: an AAV ITR, the first heterologous polynucleotide sequence including regulatory sequences, the mutated AAV ITR, the second heterologous polynucleotide in reverse orientation to the first heterologous polynucleotide and a third AAV ITR. AAV vectors comprising self-complementing genomes can be made using the methods known in the art, e.g., as described in U.S. Pat. Nos. 7,125,717; 7,785,888; 7,790,154; 7,846,729; 8,093,054; and 8,361,457.

In some embodiments, the polynucleotide molecules in the AAV vectors provided herein is less than about 5 kilobases (kb) in size. In some embodiments, the polynucleotide molecules in the AAV vectors provided herein is less than about 4.5 kb in size. In some embodiments, the polynucleotide molecules in the AAV vectors provided herein is less than about 4.0 kb in size. In some embodiments, the polynucleotide molecules in the AAV vectors provided herein is less than about 3.5 kb in size. In some embodiments, the polynucleotide molecules in the AAV vectors provided herein is less than about 3.0 kb in size. In some embodiments, the polynucleotide molecules in the AAV vectors provided herein is less than about 2.5 kb in size.

In certain embodiments, host cells containing polynucleotides of the AAV vectors described above are rendered capable of providing AAV helper functions to replicate and encapsidate the polynucleotide of interest flanked by the AAV ITRs to produce AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. In some embodiments, AAV helper functions include one, or both of the major AAV ORFs, namely the rep and cap coding regions, or functional homologues thereof.

AAV helper functions can be introduced into the host cell by transfecting the host cell with an AAV helper construct either prior to, or concurrently with, the transfection of the AAV vector polynucleotide sequences. For example, AAV helper constructs can be used to provide at least transient expression of AAV rep and/or cap genes to complement missing AAV functions that are necessary for productive AAV transduction. Typically, AAV helper constructs lack AAV ITRs and can neither replicate nor package themselves. The AAV helper constructs can be in the form of, e.g., a plasmid, phage, transposon, cosmid, virus, or virion.

In certain embodiments, the host cell is also capable of providing or is provided with non AAV-derived functions or “accessory functions” to produce AAV particles. Accessory functions are non AAV-derived viral and/or cellular functions upon which AAV is dependent for its replication, such as non AAV proteins and RNAs that are required in AAV replication, including those involved in activation of AAV gene transcription, stage specific AAV m RNA splicing. AAV DNA replication, synthesis of Cap expression products and AAV capsid assembly. In some embodiments, viral-based accessory functions can be derived from a known helper virus.

In some embodiments, as a result of the infection of the host cell with a helper virus and/or an accessory function vector, a recombinant AAV virion or a recombinant AAV particle is produced, and the produced AAV virion or AAV particle is infectious, replication-defective virus, and includes an AAV protein capsid that encapsidates a heterologous nucleotide sequence of interest flanked on both sides by AAV ITRs.

AAV virions or particles can be purified from the host cell using a purification method known in the art, such as chromatography. CsCl gradients, and other methods as described, for example, in U.S. Pat. Nos. 6,989,264 and 8,137,948 and WO 2010/148143. In some embodiments, residual helper virus can be inactivated using known methods, e.g., by heating.

### 6.7. Production of AAV Capsid Proteins

Also provided herein are methods of producing AAV capsid proteins. AAV capsid protein may be expressed recombinantly, using any method known in the art, for example, using polynucleotides encoding an AAV particle described herein or an antigenic fragment thereof or an AAV capsid protein described herein or an antigenic fragment thereof.

A polynucleotide encoding an AAV capsid protein may be operably linked to regulatory expression control sequences for expression in a specific cell type, such as Sf9 or HEK cells.

Recombinant protein expression systems may include bacterial cells, yeast cells, insect cells or mammalian expression systems. Bacterial cells may be transformed with an expression vector such as pUR278, pIN, pGEX and others. Mammalian host cells may be transformed with viral vectors, e.g., adenoviral vectors. Expression in mammalian host cells allows for post-translation modifications of the protein. The efficiency of expression can be enhanced by the inclusion of appropriate transcription enhancer elements, transcription terminators, etc. Cell lines which stably express a capsid protein described herein may be used for long term production of a high yield of the recombinant capsid protein. Selectable markers such as antibiotic resistance genes allow for the selection of cells which have stably integrated the polynucleotide encoding the recombinant capsid protein. Methods of purifying recombinant expressed proteins are well known in the art and include, for example, ion exchange chromatography, affinity chromatography and others.

An AAV capsid protein may be generated by other methods known in the art, including, e.g., by chemical synthesis, by other synthetic techniques, or by other methods. For example, the sequences of any of the capsids described herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well-known solid phase peptide synthesis methods (Merrifield, J. Am. Chem. Soc., 85:2149 (1962); Stewart and Young, Solid Phase Peptide Synthesis Freeman, (San Francisco, 1969) pp. 27-62. These and other suitable production methods are within the knowledge of those of skill in the art and are not a limitation of the present disclosure.

In certain embodiments, the method of making the AAV particle or AAV capsid protein described herein comprises (a) transfecting a host cell with a polynucleotide encoding the AAV particle or AAV capsid protein described herein such that the host cell expresses the AAV particle or AAV capsid protein, and (b) purifying the AAV particle or AAV capsid protein.

### 6.8. Cells

A variety of host cells can be used to express the virus particles and capsid proteins described herein. In certain embodiments, the cell is a mammalian host cell, for example, a HEK293, HEK293-T, A549, WEHI, 10T1/2, BHK, MDCK, COS1, COS7, BSC 1, BSC 40, BMT 10, VERO, W138, HeLa, 293, Jurkat, 2V6·11, Saos, C2C12, L, HT1080, HepG2, primary fibroblast, hepatocyte, and myoblast cells. In other aspect, the cell is an insect cell, for example an Sf9, SF21, SF900+, or a drosophila cell lines, mosquito cell lines, e.g., Aedes albopictus derived cell lines, domestic silkworm cell lines, e.g. Bombyx mori cell lines, Trichoplusia ni cell lines such as High Five cells or Lepidoptera cell lines such as Ascalapha odorata cell lines. Preferred insect cells are cells from the insect species which are susceptible to baculovirus infection, including High Five, Sf9, Sc301, SeIZD2109, SeUCR1, Sf900+, Sf21, BTI-TN-5B1-4, MG-1, Tn368, HzAm1, BM-N, Ha2302, Hz2E5 and Ao38. The efficiency of expression can be enhanced by the inclusion of appropriate transcription enhancer elements, transcription terminators, etc.

Suitable host cells for producing AAV particles from the polynucleotides and AAV vectors provided herein include microorganisms, yeast cells, insect cells, and mammalian cells. Typically such cells can be, or have been, used as recipients of a heterologous nucleic acid molecule and can grow in, e.g., suspension culture and a bioreactor.

### 6.9. Methods of Producing AAV Particles Using Insect Cells

Large scale production of recombinant AAV in cells, including Sf9 insect cells, has been described by Kotin R M. Large-scale recombinant adeno-associated virus production. Hum Mol Genet. 2011; 20(R1):R2-R6. doi:10.1093/hmg/ddr141. Methodology for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system. Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. A particularly suitable promoter for transcription of a nucleotide sequence encoding an AAV capsid protein is, e.g., the polyhedron promoter. However, other promoters that are active in insect cells are known in the art, e.g., the p10, p35 or IE-1 promoters, and further promoters described in the above references are also contemplated. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059.

In some embodiments, the nucleic acid construct encoding AAV in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori (Bm)NPV) (Kato et al., Appl. Microbiol. Biotechnol. 85(3):459-470 (2010)). Baculoviruses are commonly used for the infection of insect cells for the expression of recombinant proteins. In particular, expression of heterologous genes in insects can be accomplished as described in for instance U.S. Pat. No. 4,745,051; Friesen et al., Curr. Top. Microbiol. Immunol. 131:31-49. (1986); EP 127,839; EP 155,476; Miller et al., Ann. Rev. of Microbiol. 42: 177-199 (1988): Carbonell et al., Gene 73(2):409-18 (1988); Maeda et al., Nature 315(6020):592-4 (1985); Lebacq-Verheyden et al., Mol. Cell. Biol. 8(8):3129-35 (1988); Smith et al., Proc. Natl. Acad. Sci. USA. 82(24):8404-8 (1985); Miyajima et al., Gene 58(2-3):273-81 (1987); and Martin et al., DNA 7(2):99-106 (1988). Numerous baculovirus strains and variants and corresponding permissive insect host cells that can be used for protein production are described in Luckow et al., Nature Biotechnology 6:47-55 (1988), and Maeda et al., Nature 315(6020):592-4 (1985).

### 6.10. Definitions

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer. In instances where integers are required or expected, and instances of percentages, it is understood that the scope of this term includes rounding up to the next integer and rounding down to the next integer. For clarity, use herein of phrases such as “about X,” and “at least about X,” are understood to encompass and particularly recite “X.”

The terms “administer”, “administration”, or “administering” refer to the act of injecting or otherwise physically delivering a substance (e.g., a compound or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion. A composition provided herein may be delivered systemically or to a specific tissue. In certain embodiments, a composition provided herein may be administered directly to a tumor (i.e., is administered intratumorally).

The terms “antibody” and “immunoglobulin” are terms of art and can be used interchangeably herein, and refer to a molecule with an antigen binding site that specifically binds an antigen. In some embodiments, an isolated antibody (e.g., monoclonal antibody) described herein, or an antigen-binding fragment thereof, which specifically binds to a protein of interest is conjugated to one or more cell surface receptor ligands, for example, via a linker, or fused to an IGF-2 polypeptides via option spacer(s).

An “antibody fragment” includes only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, conjugate function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

“Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)2 fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.”

The terms “constant region”. “constant domain”, and “Fc”, are used interchangeably and refer to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The terms “variable region” and “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; A1-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82: U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

An “antigen” is a moiety or molecule that contains an epitope to which an antibody can specifically bind. Thus, an antigen is also is specifically bound by an antibody.

“The terms “binds,” “binds to,” “binding,” “specifically binds,” “specifically binds to,” “specifically binding,” “specifically binding to,” “specifically bound to” and grammatical variants of such terms refer to an interaction between molecules including, for example, to form a complex. Interactions can be, for example, such non-covalent interactions as hydrogen bonds, ionic bonds, hydrophobic interactions, and/or van der Waals interactions. The ratio of dissociation rate (koff) to association rate (kon) of a binding molecule to a monovalent partner, e.g., an antigen (koff/kon) is the dissociation constant KD, which is inversely related to affinity. The lower the KD value, the higher the affinity of the binding molecule, which depends on both kon and koff. The dissociation constant KD may be determined using any method provided herein or via any other method well known to those skilled in the art.

A binding molecule that specifically binds to a partner molecule can be identified, for example, by immunoassays, Octet®, Biacore®, or other techniques known to those of skill in the art. In some embodiments, a binding molecule specifically binds to a binding partner when it binds to the binding partner with a higher affinity than to any cross-reactive binding as determined using experimental techniques, such as radioimmunoassays (RIA) and enzyme linked immunosorbent assays (ELISAs). Typically, a specific or selective reaction will result in at least twice the background signal or noise and may be more than 10 times the background signal or noise. See, e.g., Fundamental Immunology 332-36 (Paul ed., 2d ed. 1989) for a discussion regarding binding specificity. In certain embodiments, the extent of binding of a binding molecule to a “non-partner” molecule, e.g., protein, is less than about 10% of the binding of the binding molecule to its particular partner, as determined, e.g., by fluorescence activated cell sorting (FACS) analysis or RIA.”

Unless otherwise indicated, any heteroatom with unsatisfied valences is assumed to have hydrogen atoms sufficient to satisfy the valences.

Throughout the specification, groups and substituents thereof may be chosen by one skilled in the field to provide stable moieties and compounds.

The terms “halo” and “halogen,” refer to F, Cl, Br, and I.

The term “cyano” refers to the group —CN.

The term “amino” refers to the group —NH₂.

The term “hydroxy” refers to the group —OH.

The term “nitro” refers to the group —NO₂.

The term “oxo” refers to the group ═O.

The term “alkyl” refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The term “haloalkyl” is intended to include both branched and straight-chain saturated aliphatic hydrocarbon groups substituted with one or more halogen atoms. For example, “C₁₋₄ haloalkyl” is intended to include C₁, C₂, C₃, and C₄ alkyl groups substituted with one or more halogen atoms. Representative examples of haloalkyl groups include, but are not limited to, —CF₃, —CCl₃, —CFCl₂, and —CH₂CF₃.

The term “fluoroalkyl” is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “alkenyl” refers to a straight or branched chain hydrocarbon radical containing from 2 to 12 carbon atoms and at least one carbon-carbon double bond.

Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond.

Exemplary such groups include ethynyl. For example, “C₂₋₆ alkynyl” denotes straight and branched chain alkynyl groups with two to six carbon atoms.

The term “cycloalkyl,” as used herein, refers to a group derived from a saturated monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “cycloalkenyl,” as used herein, refers to a group derived from a non-aromatic monocyclic or polycyclic hydrocarbon molecule having at least one carbon-carbon double bond, by removal of one hydrogen atom from a saturated ring carbon atom. Representative examples of cycloalkenyl groups include, but are not limited to, cyclobutenyl, cyclopentenyl, and cyclohexenyl. When numbers appear in a subscript after the symbol “C”, the subscript defines with more specificity the number of carbon atoms that a particular cycloalkyl group may contain. For example, “C₄ cycloalkenyl” denotes cycloalkenyl groups with four to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “haloalkoxy” and “—O(haloalkyl)” represent a haloalkyl group as defined above attached through an oxygen linkage (—O—). For example, “C₁₋₄ haloalkoxy” is intended to include C₁, C₂, C₃, and C₄ haloalkoxy groups.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The terms “hydroxyalkoxy” and “—O(hydroxyalkyl)” represent a hydroxyalkyl group as defined above attached through an oxygen linkage (—O—). For example, “C₁₋₄ hydroxyalkoxy” is intended to include C₁, C₂, C₃, and C₄ hydroxyalkoxy groups.

The term “alkylthio,” refers to an alkyl group attached to the parent molecular moiety through a sulfur atom, for example, methylthio group (—SCH₃). For example, “C₁₋₃ alkylthio” denotes alkylthio groups with one to three carbon atoms.

The term “arylthio,” refers to an aryl group attached to the parent molecular moiety through a sulfur atom, for example, phenylthio group (—S(phenyl)).

The terms “carbocycle”, “carbocyclo”, “carbocyclic” or “carbocyclyl” are used interchangeably and refer to cyclic groups having at least one saturated or partially saturated non-aromatic ring wherein all atoms of all rings are carbon. The carbocyclyl ring may be unsubstituted or may contain one or more substituents as valence allows. Thus, the term includes nonaromatic rings such as for example, cycloalkyl, cycloalkenyl, and cycloalkynyl rings. Exemplary bicyclic carbocyclyl groups include, indanyl, indenyl, dihydronaphthalenyl, tetrahydronaphthenyl, hexahydronaphthalenyl, octahydronaphthalenyl, decahydronaphthalenyl, bicycloheptanyl, bicyclooctanyl, and bicyclononanyl.

The term “aryl” refers to a group of atoms derived from a molecule containing aromatic ring(s) by removing one hydrogen that is bonded to the aromatic ring(s). Heteroaryl groups that have two or more rings must include only aromatic rings. Representative examples of aryl groups include, but are not limited to, phenyl and naphthyl. The aryl ring may be unsubstituted or may contain one or more substituents as valence allows.

The term “benzyl” refers to a methyl group in which one of the hydrogen atoms is replaced by a phenyl group. The phenyl ring may be unsubstituted or may contain one or more substituents as valence allows.

The term “aryloxy” refers to an aryl group attached to the parent molecular moiety through an oxygen atom, for example, phenoxy group (—O(phenyl)).

The term “heteroatom” refers to oxygen (O), sulfur (S), and nitrogen (N).

The terms “heterocycle”, “heterocyclo”, “heterocyclic”, and “heterocyclyl” are used interchangeably and refer to cyclic groups having at least saturated or partially saturated non-aromatic ring and wherein one or more of the rings have at least one heteroatom (O, S or N), said heteroatom containing ring preferably having 1 to 3 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include pyrrolidinyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, dihydroisoindolyl, and tetrahydroquinolinyl.

The term “heteroaryl” refers to substituted and unsubstituted aromatic 5- or 6-membered monocyclic groups and 9- or 10-membered bicyclic groups that have at least one heteroatom (0, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl,

benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The term “spirocarbocyclo”, “spirocarbocyclic”, or “spirocarbocyclyl” refers to a carbocyclyl ring attached to the molecular moiety by a carbon atom in the carbocyclyl ring that is shared with the molecular moiety.

The term “spiroheterocyclo”, “spiroheterocyclic”, or “spiroheterocyclyl” refers to a heterocyclyl ring attached to the molecular moiety by a carbon atom in the heterocyclyl ring that is shared with the molecular moiety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as 2H (deuterium), 3H (tritium), 13C, 14C, 15N, 17O, 18O, 18F, 36Cl, 82Br, 123I, 124I, 125I, 129I and 131I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with 3H, 14C and/or 18F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

In the description herein, if there is any discrepancy between a chemical name and chemical structure, the chemical structure shall prevail.

The terms “chimeric” and “pseudotype” or “pseudotyped” as used herein with respect to a virus (e.g., an AAV particle), mean that the virus (e.g., AAV particle), includes sequences from different viruses, e.g., different viral serotypes. For example, a “chimeric AAV particle” may refer to an AAV particle that comprises at least one capsid protein of, or derived from, one AAV serotype, and a second capsid protein of, or derived from, another AAV serotype. A pseudotyped AAV particle, for example, may refer to an AAV particle comprising at last one capsid protein of, or derived from, one AAV serotype, and a polynucleotide comprising a sequence of, or derived from, a different AAV serotype, for example, may comprise an AAV capsid protein from one serotype and an inverted terminal repeat (“ITR”) from a different AAV serotype.

A “coding sequence” or a sequence which “encodes” a selected gene product, e.g., a polypeptide, is a nucleic acid molecule which is transcribed (in the case of DNA) into RNA and translated (in the case of mRNA) into a polypeptide when placed under the control of appropriate regulatory sequences. The gene product may be a polypeptide or an RNA. When the coding sequence encodes a polypeptide, the boundaries of the coding sequence are determined by a start codon at the 5′ terminus and a translation stop codon at the 3′ terminus. A transcription termination sequence may be located 3′ to the coding sequence.

The terms “control sequences” and “regulatory sequences” refer to nucleic acid sequences that initiate, modulate and/or terminate the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “DAR” refers to the average value of “m” or the loading of the conjugate. The number of “X” moieties per each unit of “Xn-L-” or “Xn-” is represented by “n” in the formulas depicted herein. It will be understood that loading, or DAR, is not necessarily equivalent to the number of “X” moieties per conjugate molecule. By means of example, where there is one “X” moiety per unit (n=1), and one “Xn-L-” unit per conjugate (m=1), there will be 1×1=1 “X” moiety per conjugate. However, where there are two “X” moieties per unit (n=2), and four “Xn-L-” units per conjugate (m=4), there will be 2×4=8 “X” moieties per conjugate. Accordingly, for the conjugates described herein, the total number of “X” moieties per conjugate molecule will be n×m.

The terms “effective amount” and “therapeutically effective amount” refer to an amount of a therapeutic agent (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a composition described herein to achieve a specified result.

An “epitope” refers to a localized region of an antigen to which an antibody can specifically bind. An epitope can be a linear epitope of contiguous amino acids or can comprise amino acids from two or more non-contiguous regions of the antigen.

The term “flanked” as used with respect to a sequence that is flanked by other elements, indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid comprising the transgene and a flanking element. A sequence (e.g., a transgene) that is “flanked” by two other elements (e.g., ITRs) indicates that one element is located 5′ to the sequence and the other is located 3′ to the sequence; however, there may be intervening sequences between a sequence and its “flanking” sequence.

The term “homology” refers to the percent identity between two polynucleotide or two polypeptide moieties. Two DNA sequences or two polypeptide sequences are “substantially homologous” to each other when the sequences exhibit at least about 50%, at least about 75%, at least about 80%-85%, at least about 90%, at least about 95%-98% sequence identity, at least about 99%, or any percent therebetween over a defined length of the molecules. As used herein, substantially homologous also refers to sequences showing complete identity to the specified DNA or polypeptide sequence.

The term “host cell” refers to a particular cell that may be transfected with a nucleic acid molecule and the progeny or potential progeny of such a cell. Host cells may be bacterial cells, yeast cells, insect cells or mammalian cell.

The term “identity” as used herein refers to an exact nucleotide-to-nucleotide or amino acid-to-amino acid correspondence of two polynucleotides or polypeptide sequences, respectively. Methods for determining percent identity are well known in the art. For example, percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions. Another method of establishing percent identity in the context of nucleotide sequences provided herein is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PR. Details of these programs are well known in the art. Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

IGF1R refers to insulin-like growth factor 1 receptor

The term “operatively linked.” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (expression of the open reading frame). As another example, an operatively linked protein is one in which the functional domains are placed with appropriate distance from each other to impart the intended function of each domain.

The term “pharmaceutically acceptable salt” refers to those salts of the conjugate provided herein, which are formed by the process of the present application which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences. 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “promoter” as used herein in its ordinary sense refers to a nucleotide region comprising a DNA regulatory sequence which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.). “repressible promoters” (where expression of a polynucleotide sequence operably linked to the promoter is induced by an analyte, cofactor, regulatory protein, etc.), and “constitutive promoters.”

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, by disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. In certain embodiments, a polypeptide can occur as a single chain or as two or more associated chains, e.g., may be present as a multimer, e.g., dimer, a trimer. An antibody, for example, is a polypeptide. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example, chains that are non-covalently or covalently attached. e.g., linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides, polypeptides and virus particles of interest are well-known in the art and include, for example, ion-exchange chromatography, affinity chromatography and sedimentation according to density.

The term “recombinant virus particle,” “recombinant viral particle,” or reference to a particular “viral particle” or “virus particle” as “recombinant” or “r” as used herein refers to a virus that has been genetically altered, e.g., by the deletion or other mutation of an endogenous viral gene and/or the addition or insertion of a heterologous nucleic acid construct into the polynucleotide of the particle.

A “subject” is a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder disclosed herein. In another embodiment, the subject is a mammal, e.g., a human, at risk of developing a disease or disorder provided herein. In a specific embodiment, the subject is human. The terms “subject” and “patient” are used interchangeably.

The terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease. “Treatment” or “treating” includes: (1) preventing the disease, i.e., preventing the development of the disease or causing the disease to occur with less intensity in a subject that may be exposed to or predisposed to the disease but does not yet experience or display symptoms of the disease, (2) inhibiting the disease, i.e., arresting the development, preventing or retarding progression, or reversing the disease state, (3) relieving symptoms of the disease i.e., decreasing the number of symptoms experienced by the subject, and (4) reducing, preventing or retarding progression of the disease or a symptom thereof. The terms “prevent,” “preventing,” and “prevention” refer to reducing the likelihood of the onset (or recurrence) of a disease, disorder, condition, or associated symptom(s). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a composition described herein or pharmaceutical composition thereof.

A “variant” is a polypeptide having one or more different amino acid residues as compared to a corresponding parental polypeptide sequence, or a fragment thereof having a similar or identical length to the variant. In some embodiments, a parental polypeptide sequence is the wild type or naturally occurring polypeptide sequence. In some embodiments, a variant polypeptide as used herein in connection with a polypeptide refers to a polypeptide having certain percent sequence identity to a reference polypeptide, for example, having at least about 80% amino acid sequence identity with a reference polypeptide, e.g., the corresponding full-length native sequence. Such polypeptide variants include, for instance, polypeptides wherein one or more amino acid residues are added, or deleted. In embodiments, a variant has at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, alternatively at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity, or at least about 99% amino acid sequence identity to the reference polypeptide, e.g., the corresponding full-length native sequence. In embodiments, variant polypeptides are at least about 10 amino acids in length, at least about 20 amino acids in length, at least about 30 amino acids in length, at least about 40 amino acids in length, at least about 50 amino acids in length, at least about 60 amino acids in length, at least about 70 amino acids in length, at least about 80 amino acids in length, at least about 90 amino acids in length, at least about 100 amino acids in length, at least about 150 amino acids in length, at least about 200 amino acids in length, at least about 300 amino acids in length, or more. Variants include substitutions that are conservative or non-conservative in nature. For example, the polypeptide of interest may include up to about 5-10 conservative or non-conservative amino acid substitutions, or even up to about 15-25 or 50 conservative or non-conservative amino acid substitutions, or any number between 5-50.

The term “vector” refers to a substance that is used to carry or include a nucleic acid sequence, for example, in order to introduce a nucleic acid sequence into a host cell. Vectors applicable for use include, for example, expression vectors, plasmids, phage vectors, viruses, virus capsids, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed both nucleic acid molecules can be inserted, for example, into a single expression vector or in separate expression vectors. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art. The term “vector” includes cloning and expression vehicles, as well as viral vectors.

The terms “vector genome” (vg), “genome particles” (gp), “genome equivalents,” or “genome copies” as used in reference to a viral titer, refer to the number of viral particles containing a viral genome, such as an AAV DNA genome or a polynucleotide contained in a viral particle, e.g., contained in an AAV particle described herein, regardless of infectivity or functionality. The number of genome particles in a particular preparation can be measured, for example, using the procedure set forth in Clark et al. (1999) Hum. Gene Ther., 10:1031-1039; Veldwijk et al. (2002) Mol. Ther., 6:272-278.

The terms “virus particle,” “viral particle,” “virus vector” or “viral vector” are used interchangeably herein. A “virus particle” refers to a virus capsid and a polynucleotide (DNA or RNA), which may comprise a viral genome, a portion of a viral genome, or a polynucleotide derived from a viral genome (e.g., one or more ITRs), which polynucleotide optionally comprises a transgene.

### Additional Embodiments

Aspects of this disclosure include additional embodiments that are described in the following numbered clauses:

1. A bifunctional bridging compound, comprising:

an IGF-2 polypeptide capable of binding to a cell surface receptor; and

a bridging moiety that specifically binds a target viral particle.

2. The compound of clause 1, wherein the IGF-2 polypeptide is a variant IGF-2 polypeptide having diminished or no affinity for the insulin receptor and/or IGFR1 as compared to naturally occurring human IGF-2 polypeptide.
3. The compound of clause 1 or 2, wherein the IGF-2 polypeptide is a variant IGF-2 polypeptide having enhanced affinity for a CI-M6PR as compared to naturally occurring human IGF-2 polypeptide.
4. The compound of any one of clauses 1 to 3, wherein the IGF-2 polypeptide comprises an amino acid sequence that is at least 80% identical (e.g., at least 90%, at least 95%, at least 96%, at least 97%, or at least 98% identical) to a sequence of Table 1 or 2.
5. The compound of any one of clauses 1 to 4, wherein the IGF-2 polypeptide comprises a sequence selected from SEQ ID NO: 1-13.
6. The compound of clause 5, wherein the IGF-2 polypeptide consists essentially of a sequence of SEQ ID NO: 1-6.
7. The compound of any one of clauses 1 to 6, wherein the bridging moiety specifically binds a virus capsid, virus envelope, or virus protein of the viral particle.
8. The compound of any one of clauses 1 to 7, wherein the viral particle is an adenoviral (AV) particle, an adeno-associated viral (AAV) particle, a retrovirus particle, a lentiviral (LV) particle, or herpes simplex viral particle.
9. The compound of clause 8, wherein the viral particle is an AAV particle.
10. The compound of clause 9, wherein the AAV particle is of AAV serotype AAV1, AAV2, AAV2i8, AAV3, AAV3-B. AAV4, AAV5, AAV6, AAV7, AAV8, AAVrh8, AAVrh8R, or AAV rh·8, AAV9, AAV10, AAVrh10, AAV11, AAV12, AAV13, AAV LK03, AAVrh74, AAV DJ, AAV Anc81, Anc82, Anc83, Anc84, Anc110, Anc113, Anc126, or Anc127, AAV hu·37, AAV_go·1, AAV LK03, or AAV rh74.
11. The compound of any one of clauses 1 to 10, wherein the compound is of formula (I):

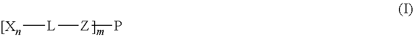

or a pharmaceutically acceptable salt thereof,
wherein:

X is the IGF-2 polypeptide;

n is 1 to 5 (e.g., n is 1 to 3, such as n is 1 or 2):

L is an optional linker;

Z is a residual linking moiety resulting from the attachment of Xₙ (or L, if present) to P via a chemoselective ligation group;

P is the bridging moiety that is capable of binding to the target viral particle (e.g., a viral particle, viral capsid, a viral envelope or a viral protein); and

m is an integer of 1 to 20 (e.g., m is 1 to 10, such as 2 to 10 or 2 to 6).

12. The compound of any one of clauses 1 to 11, wherein the bridging moiety is an antibody or antibody fragment.
13. The compound of clause 12, wherein the bridging moiety is an antibody that specifically binds an AAV serotype.
14. The compound of clause 12, wherein the bridging moiety is an antibody that exhibits pan-reactivity against a plurality of AAV serotypes.
15. The compound of clause 12, wherein the bridging moiety is ADK8 antibody.
16. The compound of any one of clauses 11 to 15, wherein the linker L is a linear linker.
17. The compound of clause 16, wherein the bifunctional compound comprises a ratio of IGF-2 polypeptide to bridging moiety of about 2:1.
18. The compound of clause 16, wherein the bifunctional compound comprises two or more IGF-2 polypeptides each linked to the bridging moiety via a linear linker.
19. The compound of any one of clauses 11 to 15, wherein the linker L is a branched linker (i.e., n is at least 2).
20. The compound of clause 19, wherein the branched linker connects two IGF-2 polypeptides to the bridging moiety.
21. The compound of clause 19, wherein the bifunctional compound comprises a ratio of IGF-2 polypeptide to bridging moiety of about 4:1.
22. The compound of any one of clauses 12 to 21, wherein the IGF-2 polypeptide is site-specifically covalently linked to the antibody or antibody fragment.
23. The compound of clause 22, wherein IGF-2 polypeptide is covalently linked to the antibody or antibody fragment via a site-specific cysteine modification on the antibody or antibody fragment (e.g., L443C) and a thiol-reactive chemoselective ligation group.
24. The compound of any one of clauses 12 to 21, wherein the IGF-2 polypeptide is covalently linked to the antibody or antibody fragment via one or more lysine residues of the antibody or antibody fragment and an amine-reactive chemoselective ligation group.
25. The compound of any one of clauses 12 to 14, wherein the bifunctional compound is a fusion protein comprising the IGF-2 polypeptide and the antibody or antibody fragment bridging moiety.
26. The compound of clause 25, further comprising a spacer polypeptide (e.g., an intervening amino acid sequence) between the IGF-2 polypeptide and the antibody or antibody fragment bridging moiety.
27. The compound of clause 25 or 26, wherein the IGF-2 polypeptide is fused to the antibody or antibody fragment bridging moiety via its C-terminal amino acid residue.
28. The compound of clause 25 or 26, wherein the IGF-2 polypeptide is fused to the antibody or antibody fragment bridging moiety via its N-terminal amino acid residue.
29. A method of viral transduction, comprising contacting a cell with a modified viral composition comprising a complex of:

(i) a viral particle; and

(ii) a bifunctional bridging compound, comprising:
an IGF-2 polypeptide capable of binding to a cell surface receptor; and
a bridging moiety that specifically binds the viral particle;

to transduce the cell with the modified viral composition.

30. The method of clause 29, wherein transduction efficiency of the viral particle into a cell is increased compared to transduction efficiency of a viral particle alone.
31. The method of clause 30, wherein the transduction efficiency is increased by 5% or more (e.g., 10%, 15%, 20%, 25% or 30% or more, or 2-fold or more, 3-fold or more, 4-fold or more, 5-fold or more, or 10-fold or more) compared to transduction efficiency of a viral particle alone.
32. The method of any one of clauses 29 to 31, wherein the transduced cell is a virus transduction-resistant cell.
33. The method of clause 32, wherein the transduced cell is an AAV transduction-resistant cell.
34. The method of any one of clauses 29 to 33, wherein the transduced cell is a mammalian cell.
35. The method of clause 34, wherein the transduced cell is a muscle cell, neural cell, liver cell, cardiac cell, lung cell, immune cell, or kidney cell.
36. The method of any one of clauses 29 to 35, wherein the cell surface receptor is a cation independent mannose-6-phosphate receptor (CI-M6PR).
37. The method of any one of clauses 29 to 36, wherein the IGF-2 polypeptide is a variant IGF-2 polypeptide having diminished or no affinity for the insulin receptor and/or IGFR1 as compared to naturally occurring human IGF-2 polypeptide.
38. The method of any one of clauses 29 to 36, wherein the IGF-2 polypeptide is a variant IGF-2 polypeptide having enhanced affinity for a CI-M6PR as compared to naturally occurring human IGF-2 polypeptide.
39. The method of any one of clauses 29 to 38, wherein the IGF-2 polypeptide comprises an amino acid sequence that is at least 80% identical (e.g., at least 90%, at least 95%, at least 96%, at least 97%, or at least 98% identical) to a sequence of Table 1.
40. The method of any one of clauses 29 to 39, wherein the IGF-2 polypeptide comprises a sequence selected from SEQ ID NO: 1-13.
41. The method of clause 40, wherein the IGF-2 polypeptide consists essentially of a sequence of SEQ ID NO: 1-6.
42. The method of any one of clauses 29 to 41, wherein the bridging moiety specifically binds a virus capsid, virus envelope, or virus protein of the viral particle.
43. The method of any one of clauses 29 to 42, wherein the viral particle is an adenoviral (AV) particle, an adeno-associated viral (AAV) particle, a retrovirus particle, a lentiviral (LV) particle, or herpes simplex viral particle.
44. The method of clause 43, wherein the viral particle is an AAV particle.
45. The method of clause 44, wherein the AAV particle is of AAV serotype AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV11, AAV12. AAV13, AAV LK03, AAVrh74, AAV DJ, AAV Anc81, Anc82, Anc83, Anc84, Anc110, Anc113, Anc126, or Anc127, AAV hu·37, AAV_go·1, AAV LK03, or AAV rh74.
46. The method of any one of clauses 29 to 45, wherein the bridging moiety is an antibody or antibody fragment.
47. The method of clause 46, wherein the bridging moiety is an antibody that specifically binds an AAV serotype.
48. The method of clause 46, wherein the bridging moiety is an antibody that exhibits pan-reactivity against a plurality of AAV serotypes.
49. The method of clause 46, wherein the bridging moiety is ADK8 antibody.
50. The method of any one of clauses 29 to 49, wherein the IGF-2 polypeptide is covalently linked to the bridging moiety via a linker (e.g., a chemoselective ligation linker as described herein).
51. The method of clause 50, wherein the linker is a linear linker.
52. The method of clause 51, wherein the bifunctional compound comprises a ratio of IGF-2 polypeptide to bridging moiety of about 2:1.
53. The method of clause 51, wherein the bifunctional compound comprises two or more IGF-2 polypeptides each linked to the bridging moiety via a linear linker.
54. The method of clause 50, wherein the linker is a branched linker.
55. The method of clause 54, wherein the branched linker connects two or more IGF-2 polypeptides to the bridging moiety.
56. The method of clause 54, wherein the bifunctional compound comprises a ratio of IGF-2 polypeptide to bridging moiety of about 4:1.
57. The method of any one of clauses 46 to 56, wherein the IGF-2 polypeptide is site-specifically covalently linked to the antibody or antibody fragment.
58. The method of clause 57, wherein the IGF-2 polypeptide is covalently linked to the antibody or antibody fragment via a site-specific cysteine modification on the antibody or antibody fragment (e.g., L443C) and a thiol-reactive chemoselective ligation group.
59. The method of any one of clauses 46 to 56, wherein IGF-2 polypeptide is covalently linked to the antibody or antibody fragment via one or more lysine residues of the antibody or antibody fragment and an amine-reactive chemoselective ligation group.
60. The method of any one of clauses 46 to 49, wherein the bifunctional compound is a fusion protein comprising the IGF-2 polypeptide and the antibody or antibody fragment bridging moiety.
61. The method of clause 60, further comprising a spacer polypeptide between the IGF-2 polypeptide and a proteinaceous target-binding moiety.
62. The method of clause 60 or 61, wherein the IGF-2 polypeptide is fused to the antibody or antibody fragment bridging moiety via its C-terminal amino acid residue.
63. The method of clause 60 or 61, wherein the IGF-2 polypeptide is fused to the antibody or antibody fragment bridging moiety via its N-terminal amino acid residue.
64. The method of any one of clauses 29 to 63, wherein the viral particle comprises a heterologous nucleic acid.
65. The method of clause 64, wherein the viral particle comprises a transgene.
66. The method of clause 64 or 65, wherein the viral particle is an AAV particle.
67. The method of clause 66, wherein the AAV particle comprises a polynucleotide comprising a transgene and at least one inverted terminal repeat (ITR).
68. The method of clause 67, wherein the polynucleotide comprises at least an ITR 5′ of the transgene (a “5′ ITR”) or an ITR 3′ of the transgene (a “3′ ITR”).
69. The method of clause 68, wherein the polynucleotide comprises a transgene flanked by a 5′ ITR and a 3′ ITR.
70. The method of any one of clauses 65 to 69, wherein the transgene is expressed in the transduced cell.
71. The method of clause 70, wherein the transgene encodes a polypeptide or RNA.
72. The method of clause 71, wherein the transgene encodes a polypeptide that is an AAT (alpha-1 anti-trypsin) polypeptide, an ADCC (aromatic L-amino acid decarboxylase) polypeptide, an antibody or an antigen-binding fragment of an antibody, a dystrophin polypeptide, a Factor VIII polypeptide, a Factor IX polypeptide, a GAA (acid alpha-glucosidase) polypeptide, a GAD (glutamate decarboxylase) polypeptide, a GDNF (glial cell line-derived neurotrophic factor) polypeptide, an ND4 (NADH dehydrogenase 4) polypeptide, a REP1 (Rab-escort protein 1) polypeptide, a REP65 (Retinal pigment epithelium-specific 65) polypeptide, a RPGR (retinitis pigmentosa GTPase regulator) polypeptide, a SERCA2a (sarcoplasmic reticulum calcium ATPase) polypeptide, an SMN (survival motor neuron) polypeptide, an anti-VEGF polypeptide, a VEGF-binding polypeptide, a TNFR (tumor necrosis factor receptor) polypeptide or a telomerase polypeptide.
73. The method of clause 70, wherein the transgene expression is achieved by administering a vector genome (vg) dose of the modified viral composition that is less than the dose that would be required of a viral composition comprising the viral particle alone.
74. The method of any one of clauses 29 to 73, wherein the modified viral composition exhibits tropism for at least one cell type or tissue when compared to a viral composition comprising a viral particle alone.
75. The method of clause 74, wherein the at least one cell type or tissue is liver.
76. The method of clause 74, wherein the modified viral composition has increased ability to transduce at least one tissue as compared to a viral composition comprising a viral particle alone.
77. The method of clause 76, wherein the at least one tissue is muscle tissue.
78. The method of clause 29, wherein the modified viral composition does not exhibit tropism for the cell.
79. The method of any one of clauses 29 to 78, wherein the contacting comprises pre-incubating the viral particle and the bifunctional bridging compound to produce the modified viral composition and combining the modified viral composition with a biological system comprising the cell.
80. The method of any one of clauses 29 to 79, wherein the contacting occurs in the presence of neutralizing antibodies.
81. A method of viral transduction, comprising administering to a subject a pharmaceutical composition comprising a modified viral composition, comprising:

(i) a viral particle; and

(ii) a bifunctional bridging compound, comprising:
an IGF-2 polypeptide capable of binding to a cell surface receptor; and
a bridging moiety that specifically binds the viral particle;

wherein the modified viral composition enters a target cell in the subject to generate a transduced cell.

82. A method of viral transduction, comprising:

(a) contacting a target cell from a subject ex vivo with a modified viral composition to generate a transduced cell, wherein the modified viral composition comprises:
(i) a viral particle; and
(ii) a bifunctional bridging compound, comprising:
   an IGF-2 polypeptide capable of binding to a cell surface receptor; and
   a bridging moiety that specifically binds the viral particle; and

(b) administering the transduced cell to the subject.

83. The method of clause 81 or 82, wherein the modified viral composition exhibits tropism for at least one cell type or tissue when compared to a viral composition comprising a viral particle alone.
84. The method of clause 83, wherein the at least one cell type or tissue is liver.
85. The method of clause 83 or 84, wherein the modified viral composition has increased ability to transduce at least one tissue as compared to a viral composition comprising a viral particle alone.
86. The method of clause 85, wherein the at least one tissue is muscle tissue.
87. The method of clause 81 or 82, wherein transduction efficiency of the viral particle into a target cell is increased compared to transduction efficiency of a viral particle alone.
88. The method of clause 87, wherein the transduction efficiency is increased by 5% or more (e.g., 10%, 15%, 20%, 25% or 30% or more, or 2-fold or more, 3-fold or more, 4-fold or more, 5-fold or more, or 10-fold or more) compared to transduction efficiency of a viral particle alone.
89. The method of any one of clauses 81 to 88, wherein the transduced cell is a virus transduction-resistant cell.
90. The method of clause 89, wherein the transduced cell is an AAV transduction-resistant cell.
91. The method of any one of clauses 81 to 90, wherein the transduced cell is a mammalian cell.
92. The method of clause 91, wherein the transduced cell is a muscle cell, neural cell, liver cell, cardiac cell, lung cell, immune cell, or kidney cell.
93. The method of any one of clauses 81 to 92, wherein the cell surface receptor is a cation independent mannose-6-phosphate receptor (CI-M6PR).
94. The method of any one of clauses 81 to 93, wherein the IGF-2 polypeptide is a variant IGF-2 polypeptide having diminished or no affinity for the insulin receptor and/or IGFR1 as compared to naturally occurring human IGF-2 polypeptide.
95. The method of any one of clauses 81 to 94, wherein the IGF-2 polypeptide is a variant IGF-2 polypeptide having enhanced affinity for a CI-M6PR as compared to naturally occurring human IGF-2 polypeptide.
96. The method of any one of clauses 81 to 95, wherein the IGF-2 polypeptide comprises an amino acid sequence that is at least 80% identical (e.g., at least 90%, at least 95%, at least 96%, at least 97%, or at least 98% identical) to a sequence of Table 1 or 2.
97. The method of any one of clauses 81 to 96, wherein the IGF-2 polypeptide comprises a sequence selected from SEQ ID NO: 1-13.
98. The method of clause 97, wherein the IGF-2 polypeptide consists essentially of a sequence of SEQ ID NO: 1-6.
99. The method of any one of clauses 81 to 98, wherein the bridging moiety specifically binds a virus capsid, virus envelope, or virus protein of the viral particle.
100. The method of any one of clauses 81 to 99, wherein the viral particle is an adenoviral (AV) particle, an adeno-associated viral (AAV) particle, a retrovirus particle, a lentiviral (LV) particle, or herpes simplex viral particle.
101. The method of clause 100, wherein the viral particle is an AAV particle.
102. The method of clause 101, wherein the AAV particle is of AAV serotype AAV1, AAV2, AAV2i8, AAV3, AAV3-B. AAV4, AAV5, AAV6, AAV7, AAV8, AAVrh8, AAVrh8R, or AAV rh·8, AAV9, AAV10, AAVrh10, AAV11, AAV12, AAV13. AAV LK03, AAVrh74, AAV DJ, AAV Anc81, Anc82, Anc83, Anc84, Anc110, Anc113, Anc126, or Anc127, AAV hu·37, AAV_go·1, AAV LK03, or AAV rh74.
103. The method of any one of clauses 81 to 102, wherein the bridging moiety is an antibody or antibody fragment.
104. The method of clause 103, wherein the bridging moiety is an antibody that specifically binds an AAV serotype.
105. The method of clause 103, wherein the bridging moiety is an antibody that exhibits pan-reactivity against a plurality of AAV serotypes.
106. The method of clause 103, wherein the bridging moiety is ADK8 antibody.
107. The method of any one of clauses 81 to 106, wherein the IGF-2 polypeptide is covalently linked to the bridging moiety via a linker (e.g., a chemoselective ligation linker as described herein).
108. The method of clause 107, wherein the linker is a linear linker.
109. The method of clause 108, wherein the bifunctional compound comprises a ratio of IGF-2 polypeptide to bridging moiety of about 2:1.
110. The method of clause 108, wherein the bifunctional compound comprises two or more IGF-2 polypeptides each linked to the bridging moiety via a linear linker.
111. The method of clause 107, wherein the linker is a branched linker.
112. The method of clause 111, wherein the branched linker connects two or more IGF-2 polypeptides to the bridging moiety.
113. The method of clause 111, wherein the bifunctional compound comprises a ratio of IGF-2 polypeptide to bridging moiety of about 4:1.
114. The method of any one of clauses 81 to 113, wherein the IGF-2 polypeptide is site-specifically covalently linked to the antibody or antibody fragment.
115. The method of clause 114, wherein the IGF-2 polypeptide is covalently linked to the antibody or antibody fragment via a site-specific cysteine modification on the antibody or antibody fragment (e.g., L443C) and a thiol-reactive chemoselective ligation group.
116. The method of any one of clauses 81 to 113, wherein IGF-2 polypeptide is covalently linked to the antibody or antibody fragment via one or more lysine residues of the antibody or antibody fragment and an amine-reactive chemoselective ligation group.
117. The method of any one of clauses 103 to 106, wherein the bifunctional compound is a fusion protein comprising the IGF-2 polypeptide and the antibody or antibody fragment bridging moiety.
118. The method of clause 117, further comprising a spacer polypeptide between the IGF-2 polypeptide and a proteinaceous target-binding moiety.
119. The method of clause 117 or 118, wherein the IGF-2 polypeptide is fused to the antibody or antibody fragment bridging moiety via its C-terminal amino acid residue.
120. The method of clause 117 or 118, wherein the IGF-2 polypeptide is fused to the antibody or antibody fragment bridging moiety via its N-terminal amino acid residue.
121. The method of any one of clauses 81 to 120, wherein the viral particle comprises a heterologous nucleic acid.
122. The method of clause 121, wherein the viral particle comprises a transgene.
123. The method of clause 121 or 122, wherein the viral particle is an AAV particle.
124. The method of clause 123, wherein the AAV particle comprises a polynucleotide comprising a transgene and at least one inverted terminal repeat (ITR).
125. The method of clause 124, wherein the polynucleotide comprises at least an ITR 5′ of the transgene (a “5′ ITR”) or an ITR 3′ of the transgene (a “3′ ITR”).
126. The method of clause 125, wherein the polynucleotide comprises a transgene flanked by a 5′ ITR and a 3′ ITR.
127. The method of any one of clauses 122 to 126, wherein the transgene is expressed in the transduced cell.
128. The method of clause 127, wherein the transgene encodes a polypeptide or RNA.
129. The method of clause 128, wherein the transgene encodes a polypeptide that is an AAT (alpha-1 anti-trypsin) polypeptide, an ADCC (aromatic L-amino acid decarboxylase) polypeptide, an antibody or an antigen-binding fragment of an antibody, a dystrophin polypeptide, a Factor VIII polypeptide, a Factor IX polypeptide, a GAA (acid alpha-glucosidase) polypeptide, a GAD (glutamate decarboxylase) polypeptide, a GDNF (glial cell line-derived neurotrophic factor) polypeptide, an ND4 (NADH dehydrogenase 4) polypeptide, a REP1 (Rab-escort protein 1) polypeptide, a REP65 (Retinal pigment epithelium-specific 65) polypeptide, a RPGR (retinitis pigmentosa GTPase regulator) polypeptide, a SERCA2a (sarcoplasmic reticulum calcium ATPase) polypeptide, an SMN (survival motor neuron) polypeptide, an anti-VEGF polypeptide, a VEGF-binding polypeptide, a TNFR (tumor necrosis factor receptor) polypeptide or a telomerase polypeptide.
130. The method of clause 128, wherein the transgene expression is achieved by administering a vector genome (vg) dose of the modified viral composition that is less than the dose that would be required of a viral composition comprising the viral particle alone.
131. The method of clause 81, wherein the modified viral composition does not exhibit tropism for the cell.
132. The method of any one of clauses 81 to 131, wherein the contacting occurs in the presence of neutralizing antibodies.
133. A pharmaceutical composition comprising:

a modified viral composition comprising:
a viral particle; and
a bifunctional bridging compound, comprising:
   an IGF-2 polypeptide capable of binding to a cell surface receptor; and
   a bridging moiety that specifically binds the viral particle; and

a pharmaceutically acceptable carrier.

134. The pharmaceutical composition of clause 133, wherein the viral particle comprises a transgene.
135. The pharmaceutical composition of clause 134, wherein the transgene encodes a therapeutic polypeptide.
136. The pharmaceutical composition of clause 135, wherein the therapeutic polypeptide is an enzyme.
137. The pharmaceutical composition of clause 136, wherein the enzyme is acid alpha-glucosidase (GAA), phenylalanine ammonia-lyase, alpha-galactosidase A, glucocerebrosidase (GCase), aspartylglucosaminidase (AGA), alpha-L-iduronidase, iduronate sulfatase, sulfaminase, alpha-N-acetylglucosaminidase (NAGLU), alpha-glucosaminide N-acetyltransferase (HGSNAT), N-acetylglucosamine 6-sulfatase (GNS), N-glucosamine 3-O-sulfatase (ARSG), N-acetylgalactosamine 6-sulfatase, beta-glucuronidase, palmitoyl protein tioesterase (PPT1), tripeptidyl peptidase (TPP1), acid sphingomyelinase, or lysosomal acid lipase.
138. The pharmaceutical composition of any one of clauses 135 to 137, wherein the bifunctional bridging compound is according to any one of clauses 1 to 28.
139. The pharmaceutical composition of any one of clauses 135 to 138, wherein the viral particle is an adenoviral (AV) particle, an adeno-associated viral (AAV) particle, a retrovirus particle, a lentiviral (LV) particle, or herpes simplex viral particle.
140. The pharmaceutical composition of clause 139, wherein the viral particle is an AAV particle.
141. The pharmaceutical composition of clause 140, wherein the AAV particle is of AAV serotype AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV11, AAV12, AAV13. AAV LK03, AAVrh74, AAV DJ, AAV Anc81, Anc82, Anc83, Anc84, Anc110, Anc113, Anc126, or Anc127, AAV hu·37, AAV_go·1, AAV LK03, or AAV rh74.
142. A method of delivering a transgene to cells of a subject, comprising administering an effective amount of the pharmaceutical composition according to clause 134 to a subject in need thereof.
143. The method of clause 142, wherein the subject has previously been administered a viral composition.
144. The method of clause 142 or 143, wherein the method generates cells transduced with the viral composition in the subject.
145. The method of clause 142 or 143, wherein the effective amount of the pharmaceutical composition administered is less than the effective amount of a pharmaceutical composition comprising a viral particle alone.
146. The method of clause 142, wherein the pharmaceutical composition is according to any one of clauses 135 to 141.

### 7. EXAMPLES

The examples in this section are offered by way of illustration, and not by way of limitation.

### 7.1. Preparation of Cell Surface Receptor Ligands

The following are illustrative schemes and examples of how the ligand compounds described herein can be prepared, conjugated to bridging moieties, and tested. Although the examples can represent only some embodiments, it should be understood that the following examples are illustrative and not limiting. The reagents and starting materials are readily available to one of ordinary skill in the art. The specific synthetic steps for each of the routes described may be combined in different ways, or in conjunction with steps from different schemes, to prepare the compounds described herein.

7.1.1. Mannose-6-Phosphate Receptor (M6PR) Ligands

### Compound A. Synthesis of (2-((2R,3S,4S,5S,6R)-3,4,5-trihydroxy-6-(4-isothiocyanatophenoxy)tetrahydro-2H-pyran-2-yl)ethyl)phosphonic acid (Compound A)

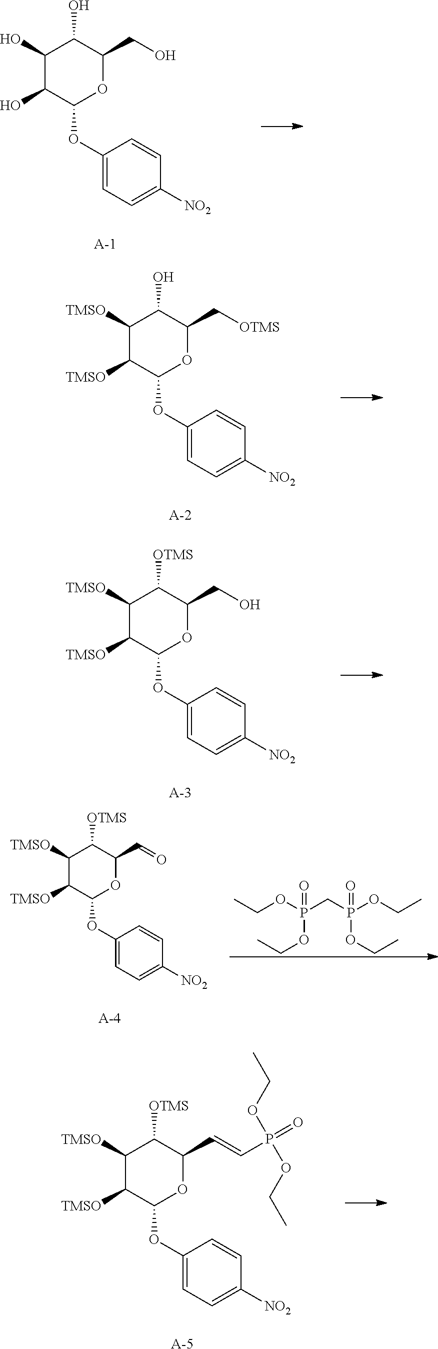

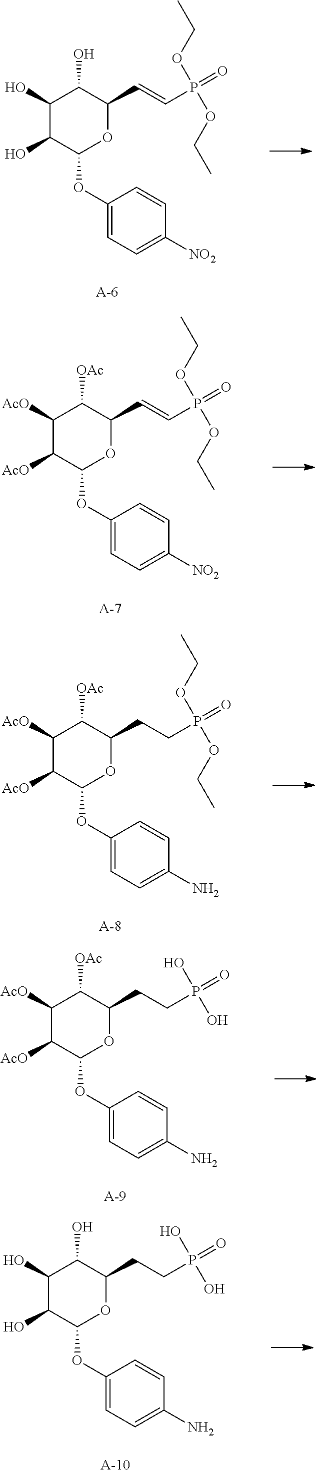

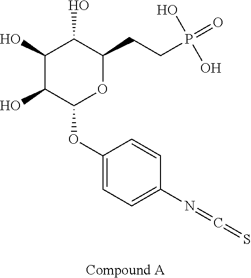 [SMILES: O=P(O)(O)CC[C@H]1O[C@H](Oc2ccc(N=C=S)cc2)[C@@H](O)[C@@H](O)[C@@H]1O ; "Compound A"]

### (((2R,3S,4S,5R,6R)-2-(4-nitrophenoxy)-6(((trimethylsilyl)oxy)methyl)tetrahydro-2H-pyran-3,4,5-triyl)tris(oxy))tris(trimethylsilane) (A-2)

A solution of (2R,3S,4S,5S,6R)-2-(hydroxymethyl)-6-(4-nitrophenoxy)tetrahydro-2H-pyran-3,4,5-triol (A-1) (1.0 eq, 26.0 g, 86.37 mmol) in DMF (500 mL) was cooled to 0° C. Then triethylamine (6.4 eq, 288 mL, 552.0 mmol) and trimethylsilyl chloride (24.0 eq 70 mL, 2071.0 mmol) were added under nitrogen atmosphere to above solution. The resulting mixture was stirred at room temperature under nitrogen for 24 h. The reaction mixture was partitioned between ethyl acetate and water. The water layer was extracted again with ethyl acetate. The combined organic layers were dried over sodium sulfate, filtered, and purified via silica gel chromatography (0 to 5% ethyl acetate in hexane) to afford Intermediate A-2 as colorless oil. Yield: 36.8 g (72.3%): ¹H NMR (400 MHz, CDCl₃) δ 8.18 (dd, J=12.36, 3.16 Hz, 2H), 7.16 (dd, J=12.4, 3.12 Hz, 2H), 5.37 (d, J=2.36 Hz, 1H), 3.99-3.87 (m, 3H), 3.72-3.69 (m, 2H), 3.50-3.48 (m, 1H), 0.2-0.07 (m, 36H).

### ((2R,3R,4S,5S,6R)-6-(4-nitrophenoxy)-3,4,5-tris((trimethylsilyl)oxy)tetrahydro-2H-pyran-2-yl)methanol (A-3)

To a stirred solution of Intermediate A-2 (1.0 eq, 10.0 g, 16.97 mmol) in mixture of DCM:methanol (8:2 ratio, 100 mL) ammonium acetate (1.5 eq, 1.96 g, 25.46 mmol) was added at room temperature under nitrogen. The resulting mixture was stirred at room temperature under nitrogen for 16 h. The reaction mixture was partitioned between ethyl acetate and water. The water layer was extracted again with ethyl acetate. The combined organic layers were dried over sodium sulfate, filtered, concentrated under vacuum and purified via silica gel chromatography (20-30% ethyl acetate in hexane) to afford Intermediate A-3 as white solid. Yield: 7.0 g (80%); LC-MS m/z 516.13 [M-1].

### (2S,3R,4S,5S,6R)-6-(4-nitrophenoxy)-3,4,5-tris((trimethylsilyl)oxy)tetrahydro-2H-pyran-2-carbaldehyde (A4)

To a stirred solution of oxalyl chloride (1.1 eq, 0.5 mL, 5.31 mmol) in DCM (5 mL) at −78° C. was added a solution of DMSO (2.2 eq, 0.76 mL, 10.62 mmol) in DCM (5 mL) over 5 min. After being stirred at −78° C. for 20 min, a solution of Intermediate A-3 (1.0 eq, 2.5 g, 4.83 mmol) in DCM (10 mL) was added to the mixture. The reaction mixture was further stirred at −78° C. for 60 min, followed by addition of triethylamine (5.0 eq, 3.4 mL, 24.15 mmol). The resulting mixture was allowed to reach room temperature over 1 h. The turbid mixture was diluted with DCM and washed with water followed by brine solution. The organic layer was dried over sodium sulfate, filtered, and concentrated under high vacuum to afford Intermediate A-4 as light brown gel (2.2 g, crude), which was used without further purification for the next step.

### Diethyl ((E)-2-((2R,3R,4S,5S,6R)-6-(4-nitrophenoxy)-3,4,5-tris((trimethylsilyl)oxy)tetrahydro-2H-pyran-2-yl)vinyl)phosphonate (A-5)

A stirred suspension of tetraethyl methylenebis(phosphonate) (1.5 eq, 1.85 g, 6.40 mmol) in dry THF (20 mL) was cooled to −78° C. and added n-BuLi in hexane 2.0 M (1.25 eq, 2.6 ml, 5.33 mmol). The resulting mixture was stirred for 1 h at −78° C., then Intermediate A-4 (1.0 eq. 2.2 g, 4.27 mmol) in dry THF (10 mL) was added at −78° C. The bath was removed and the reaction mixture was allowed to room temperature and stirring continued for 12 h. A saturated aqueous solution of NH₄Cl was added and extracted with ethyl acetate. Ethyl acetate layer washed with water followed by saturated brine solution. The organic layer was dried over sodium sulfate, filtered and concentrated. The crude was purified via silica gel chromatography (30-40% ethyl acetate in hexane) to afford Intermediate A-5 as colorless gel. Yield (1.3 g, 48%); LC-MS m/z 650.57 [M+1]⁺.

### Diethyl ((E)-2-((2R,3S,4S,5S,6R)-3,4,5-trihydroxy-6-(4-nitrophenoxy)tetrahydro-2H-pyran-2-yl)vinyl)phosphonate (A-6)

To a stirred solution of Intermediate A-5 (1.0 eq, 1.3 g, 1.54 mmol) in methanol (15 mL) was added Dowex 50WX8 hydrogen form at room temperature under nitrogen atmosphere. The resulting mixture was stirred at room temperature under nitrogen for 2 h. The reaction mixture filtered and washed with methanol, filtrate concentrated under vacuum to afford diethyl ((E)-2-((2R,3S,4S,5S,6R)-3,4,5-trihydroxy-6-(4-nitrophenoxy)tetrahydro-2H-pyran-2-yl)vinyl)phosphonate (6) as white solid Yield: 0.78 g (90%); LC-MS m/z 434.17 [M+1]⁺.

### (2R,3R,4S,5S,6R)-2-((E)-2-(diethoxyphosphoryl)vinyl)-6-(4-nitrophenoxy)tetrahydro-2H-pyran-3,4,5-triyl triacetate (A-7)

To a stirred solution of Intermediate A-6, (1.00 eq, 0.78 g, 1.80 mmol) in pyridine (10 mL) was added an acetic anhydride (10.0 eq. 1.8 mL, 18.0 mmol) dropwise at 0° C. under nitrogen. The cold bath was removed and the resulting mixture was stirred at room temperature under nitrogen for 16 h. Pyridine was removed on a high vacuum and the residue was partitioned between ethyl acetate and aqueous 1N HCl. The water layer was extracted again with ethyl acetate. The combined organic layers were dried over sodium sulfate, filtered, concentrated and purified via silica gel chromatography (2.5% methanol in dichloromethane) to afford Intermediate A-7 as white solid. Yield: 1.0 g (100%); LC-MS m/z 560.17 [M+1]⁺.

### (2R,3S,4S,5R,6R)-2-(4-aminophenoxy)-6-(2-(diethoxyphosphoryl)ethyl)tetrahydro-2H-pyran-3,4,5-triyl triacetate (A-8)

To a stirred solution of Intermediate A-7 (1.0 eq, 1.0 g, 1.78 mmol) in methanol (15 mL) 10% palladium on carbon (0.200 g) was added at room temperature under nitrogen. The resulting mixture was stirred at room temperature under hydrogen gas pressure (100 psi) for 16 h. The reaction mixture filtered through Celite bed and washed with methanol, filtrate concentrated under vacuum to afford Intermediate A-8 as brown sticky gel. Yield: 0.700 g (73.6%); LC-MS m/z 532.21 [M+1]⁺.

### (2-((2R,3R,4S,5S,6R)-3,4,5-triacetoxy-6-(4-aminophenoxy)tetrahydro-2H-pyran-2-yl)ethyl)phosphonic acid (A-9)

To a stirred solution of Intermediate A-8 (1.00 eq, 2.0 g, 5.73 mmol) in acetonitrile (15 mL) bromotrimethylsilane (5.0 eq, 3.8 mL, 28.65 mmol) was added dropwise at 0° C. under nitrogen. The cold bath removed and the resulting mixture was stirred at room temperature under nitrogen for 16 h. Volatiles were removed on a rotary evaporator and the residue was dried under high vacuum. The crude residue was triturated with diethyl ether and dried under high vacuum to afford Intermediate A-9 as brown solid. Yield: 2.2 g, crude. LC-MS m/z 476.0 [M+1]⁺.

### (2-((2R,3S,4S,5S,6R)-6-(4-aminophenoxy)-3,4,5-trihydroxytetrahydro-2H-pyran-2-yl)ethyl)phosphonic acid (A-10)

To a stirred solution of Intermediate A-9 (1.0 eq. 2.0 g, 4.21 mmol) in mixture of methanol:water (8:2, 15 mL) triethylamine (5.0 eq, 2.93 mL, 21.05 mmol) was added dropwise at 0° C. under nitrogen. The cold bath removed and the resulting mixture was stirred at room temperature for 16 h. Methanol was removed on a rotary evaporator and the residue was dried under high vacuum. The residue was taken up in water and purified via preparatory HPLC (2-10% acetonitrile in water with 5 mM ammonium acetate). Fractions containing the desired product were combined and lyophilized to dryness to afford Intermediate A-10 as brown solid. Yield: 0.350 g (25%); LC-MS m/z 348.0 [M-H]⁻.

### (2-((2R,3S,4S,5S,6R)-3,4,5-trihydroxy-6-(4-isothiocyanatophenoxy)tetrahydro-2H-pyran-2-yl)ethyl)phosphonic acid (Compound A)

To a stirred solution of Intermediate A-10 (1.0 eq, 1.75 g, 5.01 mmol) in mixture of ethanol:water (7:3) (20 ml) was added thiophosgene (5.00 eq. 1.92 mL, 25.05 mmol) dropwise at 0° C. under nitrogen. The cold bath removed and the resulting mixture was stirred at room temperature under nitrogen for 3 h. Volatiles were removed on a rotary evaporator and the residue was dried under high vacuum. The residue was taken up in water and purified via prep-HPLC (20-40% acetonitrile in water with 5.0 mmol ammonium acetate). Fractions containing the desired product were combined and lyophilized to dryness to afford Compound A as a white solid. Yield: 0.135 g (6.8%) LC-MS m/z 392.08 [M+1]⁺; ¹H NMR (400 MHz, D₂O) δ 7.32 (d, J=8.92 Hz, 2H), 7.12 (d, J=8.96 Hz, 2H), 5.57 (s, 1H), 4.13 (s, 1H), 3.96 (dd, J=9.16, 3.44 Hz, 1H), 3.59-3.48 (m, 2H), 2.03-1.88 (m, 1H), 1.68-1.54 (m, 2H), 1.27-1.15 (m, 1H).

Synthesis of Compound I-7

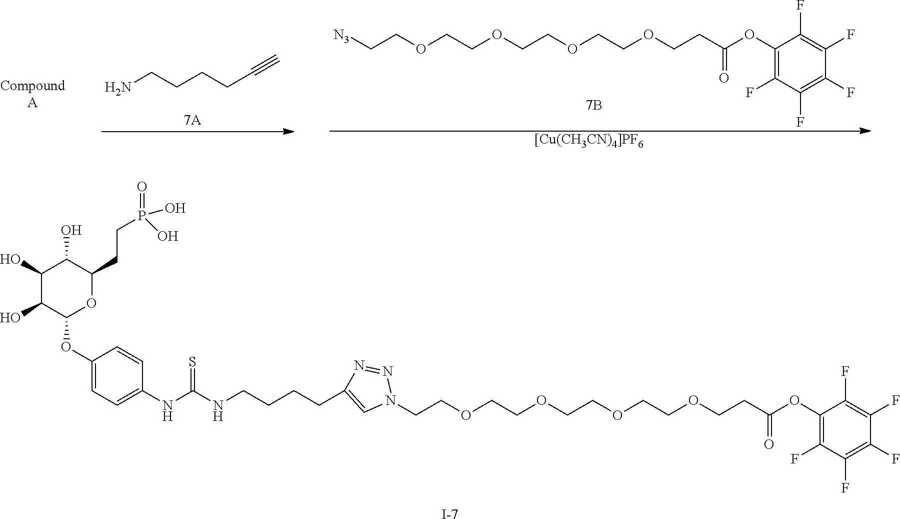

A solution of hex-5-yn-1-amine (7A) (1.20 eq. 3.9 mg, 0.0405 mmol) in NMP (0.15 mL) was added to Compound A (1.00 eq, 13.2 mg, 0.0337 mmol) in a 1 dram vial with a stirbar. The resulting mixture was capped and stirred at room temperature for 30 min (Solids slowly dissolved to give a clear yellow solution). A solution of azido-PEG4-pentafluorophenol ester (7B) (1.50 eq, 23.1 mg, 0.0506 mmol) in NMP (0.20 mL) was added followed by tetrakis(acetonitrile)copper(I) hexafluorophosphate (3.00 eq, 37.7 mg, 0.101 mmol). The resulting clear dark yellow solution was capped and stirred at room temperature for 30 min. The reaction mixture was diluted with mixture of NMP, ethanol, and acetic acid, filtered, and purified via preparatory HPLC (15-60% acetonitrile in water with 0.1% TFA). Fractions containing the desired product were combined and lyophilized to dryness to afford Compound I-7 as a white solid. Yield: 11.1 mg, 35%; LC-MS m/z 946.5 [M+1]+; ¹H NMR (300 MHz, DMSO-d₆ with D₂O) δ 7.80 (s, 1H), 7.25 (d, J=8.4 Hz, 2H), 6.98 (d, J=8.4 Hz, 2H), 5.32 (s, 1H), 4.44 (s, 2H), 3.86-3.68 (m, 5H), 3.67-3.23 (m, 17H), 3.05-2.91 (m, 2H), 2.67-2.56 (m, 2H), 2.00-1.81 (m, 1H), 1.69-1.41 (m, 6H), 1.30-1.07 (m, 1H).

7.1.2. Synthesis of Other Compounds

The synthetic methods described above can be adapted to prepare a variety of M6PR ligand-linker compounds. Several compounds were prepared for use in conjugations, e.g., to a bridging moiety such as an antibody. Full details of the synthetic methods and compounds prepared are disclosed in International Application No. PCT/US2021/012846, filed Jan. 8, 2021 and entitled “Cell Surface Receptor Binding Compounds and Conjugates”, the disclosure of which is incorporated herein by reference in its entirety. For example, compounds I-8 to I-12 were prepared as described in disclosed in International Application No. PCT/US2021/012846.

Compound I-8.

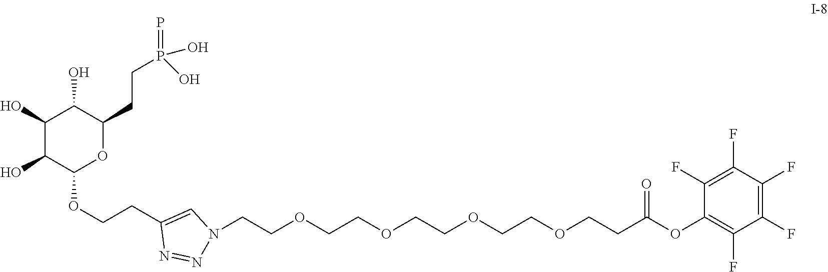

Compound I-8. LC-MS m/z 768.5 [M+1]+; ¹H NMR (300 MHz, DMSO-d₆) δ 7.81 (s, 1H), 4.59 (s, 1H), 4.44 (bs, 2H), 3.60-3.30 (m, 17H), 3.27-2.76 (m, 9H), 2.01-1.84 (m, 1H), 1.77-1.58 (m, 1H), 1.56-1.32 (m, 2H).

Compound I-9.

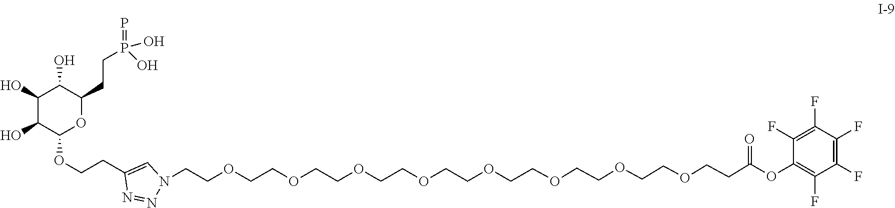

Compound I-9. LC-MS m/z 944.6 [M+1]+; 1H NMR (300 MHz, DMSO-d₄ with D₂O) δ 7.81 (s, 1H), 4.59 (s, 1H), 4.44 (s, 2H), 3.86-3.29 (m, 34H), 3.29-2.69 (m, 8H), 2.01-1.80 (m, 1H), 1.80-1.57 (m, 1H), 1.56-1.30 (m, 2H).

Compound I-10.

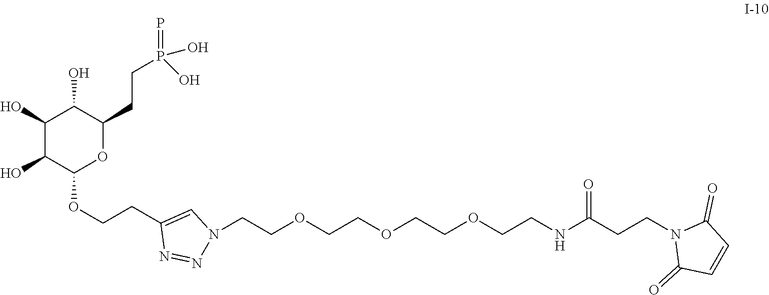

Compound I-10. LC-MS m/z 680.5 [M+1]+¹H NMR (300 MHz, DMSO-d₆ with D₂O) δ 7.81 (s, 1H), 6.92 (s, 2H), 4.59 (s, 1H), 4.44 (s, 2H), 3.63-3.26 (m, 15H), 3.26-2.70 (m, 9H), 2.36-2.21 (m, 2H), 2.05-1.83 (m, 1H), 1.79-1.60 (m, 1H), 1.54-1.30 (m, 2H).

Compound I-11

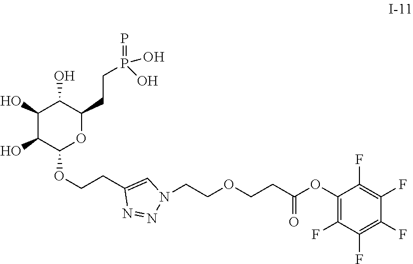

Compound I-11. LC-MS m/z 636.4 [M+1]+; ¹H NMR (300 MHz, DMSO-d₆ with D₂O) δ 7.75 (s, 1H), 4.57 (s, 1H), 4.51-4.35 (m, 2H), 3.84-3.65 (m, 5H), 3.60-3.45 (m, 2H), 3.41-3.29 (m, 1H), 3.21 (t, J=9.3 Hz, 1H), 3.15-3.03 (m, 1H), 3.03-2.88 (m, 2H), 2.88-2.74 (m, 2H), 2.02-1.82 (m, 1H), 1.79-1.59 (m, 1H), 1.56-1.28 (m, 2H).

Compound I-12.

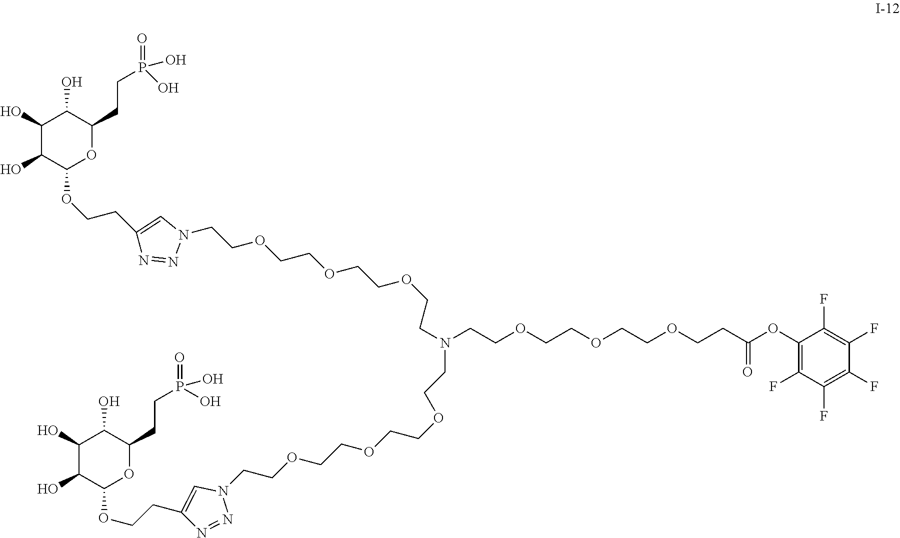 [SMILES: O=C(CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F]

Compound I-12 as a white solid. Yield: 8.7 mg, 21%; LC-MS m/z 1410.9 [M+1]⁺; ¹H NMR (300 MHz, DMSO-d₆ with D₂O) δ 7.81 (s, 2H), 4.60 (s, 2H), 4.45 (s, 4H), 3.87-2.76 (m, 50H), 2.03-1.83 (m, 2H), 1.79-1.59 (m, 2H), 1.55-1.29 (m, 4H).

### 7.2. ASGPR Ligand-Linkers

### Synthesis of [(2R,3R,4R,5R,6R)-3,4-bis(acetyloxy)4-(but-3-yn-1-yloxy)-5-acetamidooxan-2-yl]methyl acetate (Intermediate A)

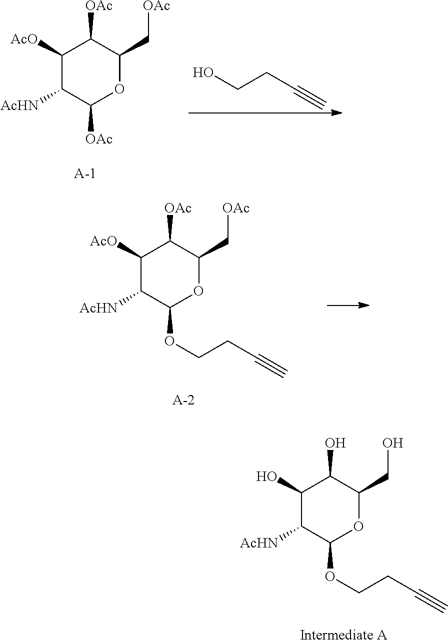

To an activated 4 Å molecular sieves (5.0 g) and [(2R,3R,4R,5R,6S)-3,4,6-tris(acetyloxy)-5-acetamidooxan-2-yl]methyl acetate (A-1) (5.0 g, 12.8 mmol), was added dichloromethane (50 mL) and stirred at room temperature for 5 min followed by addition of but-3-yn-1-ol (2.92 mL, 3.0 eq., 38.5 mmol). Stirred the reaction mixture for 10 min at room temperature and then cooled to 0° C. Diethyl trifluoroborinate (4.75 mL, 38.5 mmol) added dropwise to above reaction mixture and again stirred for 10 min at room temperature followed by 5 h refluxing at 51° C. TLC checked for the completion of reaction and triethylamine added to quench the diethyl trifluoroborinate (up to neutral pH) and filtered through celite bed followed by concentration on rotary evaporator. Obtained thick residue was purified by silica gel column purification with 60-75% ethyl acetate in dichloromethane as eluent that afforded Intermediate A-2 as an off white foam. Yield: 4.50 g, 87%; R_{f}=0.45 (7.5% methanol in dichloromethane); LC-MS m/z 400.0 [M+1]⁺; ¹H NMR (400 MHz, CDCl₃) δ 5.44 (d, J=8.6 Hz, 1H), 5.35 (d, J=7.0 Hz, 1H), 5.30 (dd, J=11.2, 3.0 Hz, 1H), 4.79 (d, J=8.2 Hz, 1H), 4.14-4.09 (m, 2H), 3.99-3.90 (m, 3H), 3.71-3.65 (m, 1H), 2.49-2.47 (m, 2H), 2.14 (s, 3H), 2.05 (s, 3H), 2.00 (s, 3H), 1.96 (s, 3H).

Intermediate A-2 (7.8 g, 17.5 mmol) was dissolved in methanol (50 mL) and cooled to 0° C. Sodium methoxide 25% w/v (2.48 mL, 11.3 mmol) in methanol added drop-wise to this solution and reaction maintained at room temperature for 3 h. TLC Checked and after completion of reaction IN HCl was added drop-wise to quench the sodium methoxide. Methanol evaporated and obtained residue was washed with diethyl ether (30 mL×4). The crude residue obtained was purified with prep-HPLC (5-20% acetonitrile in water with 0.1% TFAH) to afford Intermediate A as a white solid. Yield: 2.6 g, 84%; LC-MS m/z 274.0 [M+1]⁺; ¹H NMR (400 MHz, D₂O) δ 4.58 (d, J=8.4 Hz, 1H), 3.97-3.86 (m, 3H), 3.82-3.73 (m, 5H), 2.49-2.44 (m, 2H), 2.04 (s, 3H).

### Synthesis of N-((2R,3R,4R,5R,6R)-6-((but-3-yn-1-yloxy)methyl)-2,4,5-trihydroxtetrahydro-2H-pyran-3-yl)acetamide (Intermediate B)

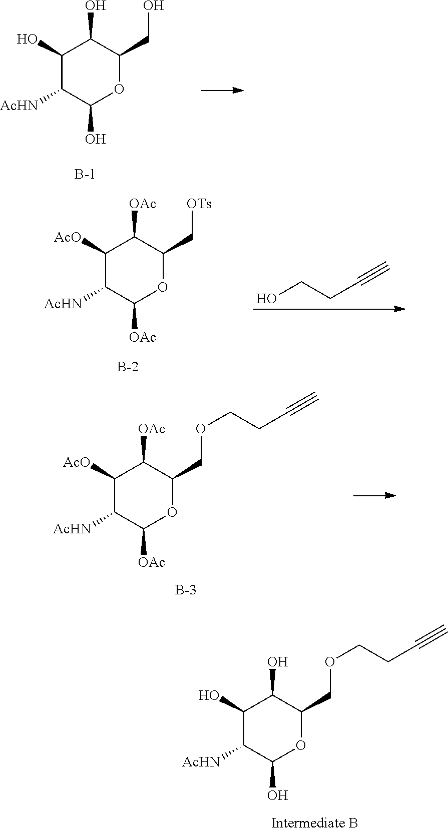

A solution of p-toluenesulfonyl chloride (1.1 eq.) in dichloromethane is added slowly to a stirred solution of N-((2R,3R,4R,5R,6R)-2,4,5-trihydroxy-6-(hydroxymethyl)tetrahydro-2H-pyran-3-yl)acetamide (B-1) (1 eq.) in dichloromethane at 0° C. The reaction mixture is warmed to room temperature and monitored by LC-MS to indicate complete formation of the desired primary alcohol tosylate. Pyridine (3.5 eq.) is added followed by acetic anhydride (3.1 eq.). The reaction mixture is stirred at room temperature and monitored by LC-MS to indicate complete formation of Intermediate B-2, which is isolated by silica gel chromatography. Sodium hydride (1.1 eq.) is added to a stirred solution of but-3-yn-1-ol (1.1 eq.) in tetrahydrofuran at 0° C. After stirring at 0° C. for 10 min a solution of Intermediate B-2 (1 eq.) in tetrahydrofuran is added. The resulting mixture is warmed to room temperature and monitored by LC-MS to indicate complete formation of Intermediate B-3, which is isolated by silica gel chromatography. Sodium methoxide in methanol (3 eq.) is added to a stirred solution of Intermediate B-3 (1 eq.) in methanol at 0° C. The resulting mixture is stirred at 0° until LC-MS indicates complete conversion to Intermediate B, which is isolated by reverse phase chromatography.

Synthesis of Trivalent GalNAc Ligand A Perfluorophenyl Ester (Compound I-107)

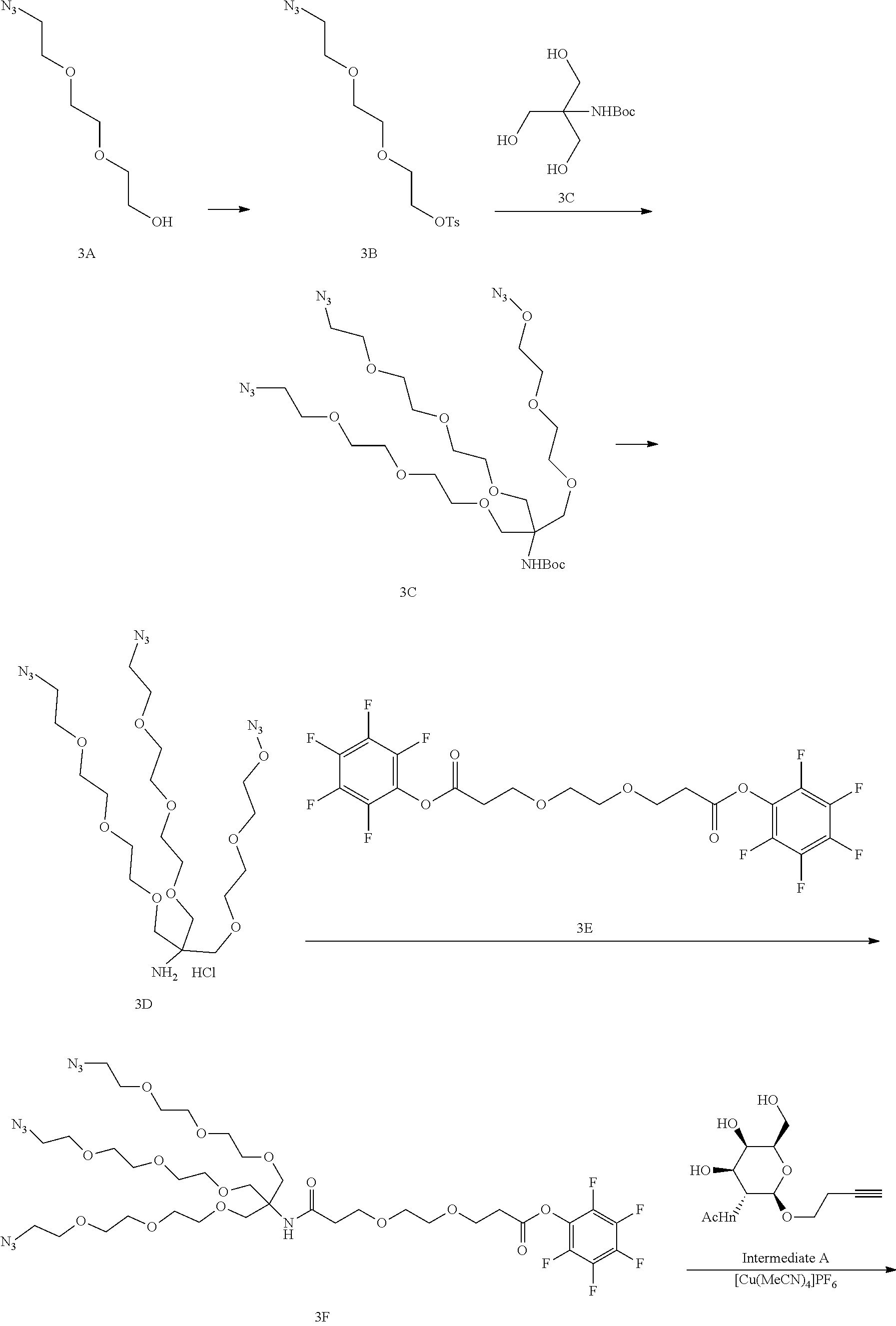

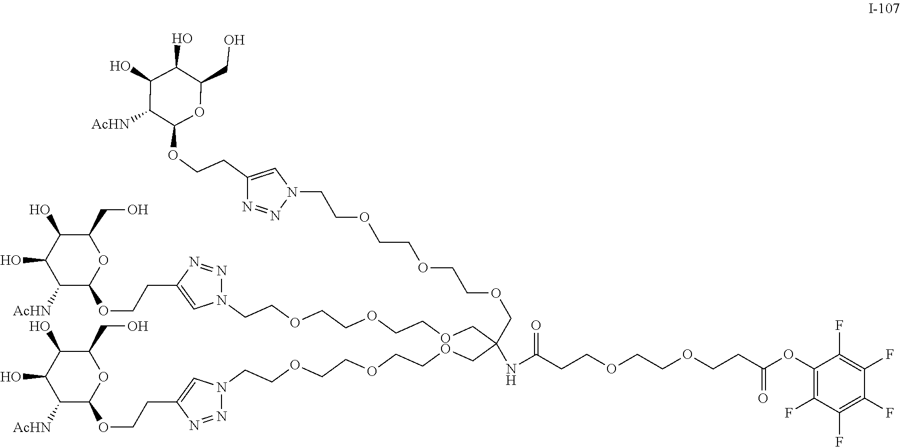 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O]

A solution of p-toluenesulfonyl chloride (1.1 eq.) in dichloromethane is added to a stirred solution of 2-(2-(2-azidoethoxy)ethoxy)ethan-1-ol (3A) (1 eq.) and pyridine (1.2 eq.) in dichloromethane. The resulting mixture is stirred at room temperature and monitored by LC-MS to indicate complete formation of Compound 3B, which is isolated by silica gel chromatography. Sodium hydride is added to a stirred mixture of tert-butyl (1,3-dihydroxy-2-(hydroxymethyl)propan-2-yl)carbamate (3C) (1 eq.) and Compound 3B (3.3 eq.) in THF at −78° C. The cold bath is removed and the resulting mixture is stirred at room temperature until LC-MS indicates complete conversion to Compound 3C, which is isolated by silica gel chromatography. HCl in diethyl ether (3 eq.) is added to a stirred solution of tert-Compound 3C (1 eq.) in dichloromethane at room temperature. The resulting mixture is stirred at room temperature until LC-MS indicates complete conversion and then volatiles are removed on a rotary evaporator to afford Compound 3D. Diisopropylethylamine (2 eq.) is added to a stirred solution of Compound 3D (1 eq.) in dichloromethane at room temperature. Bis(perfluorophenyl) 3,3′-(ethane-1,2-diylbis(oxy))dipropionate (3E) (1.1 eq.) is added and the resulting mixture is stirred at room temperature until LC-MS indicates complete conversion to Compound 3F, which is isolated by silica gel chromatography. Compound 3F (1 eq.) and Intermediate A (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-107, which is purified via reverse-phase preparatory HPLC followed by lyophilization.

Synthesis of Compound I-122

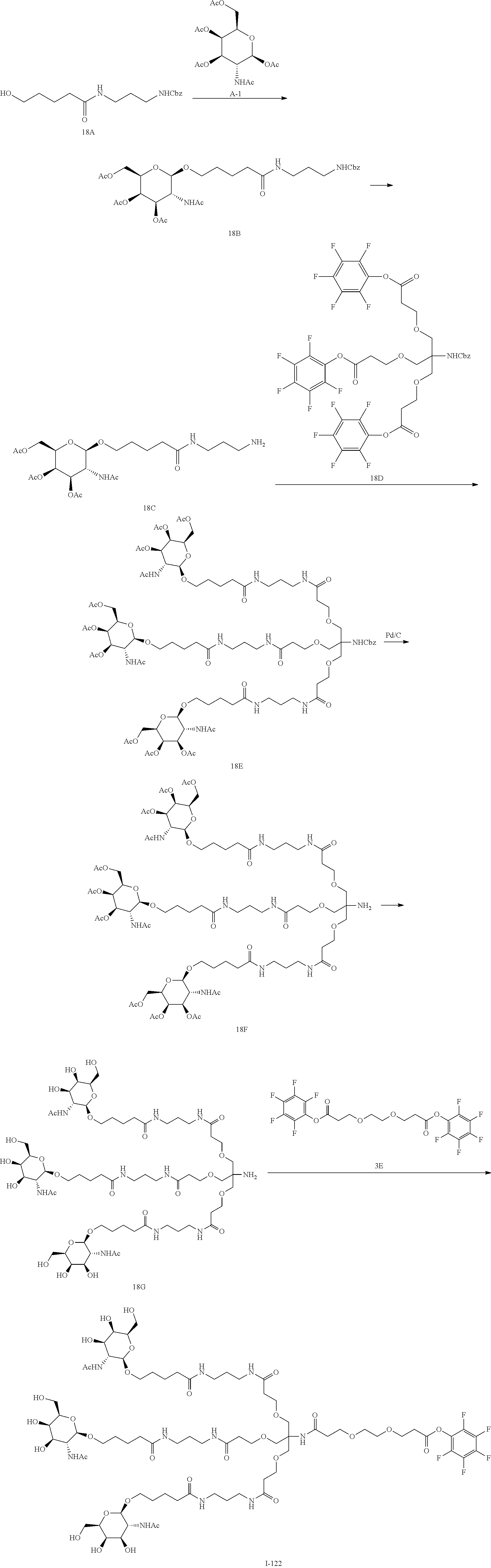

To the solution of Compound A-1 (1.0 eq, 5.05 g, 13.0 mmol) and benzyl N-[3-(5-hydroxypentanamido) prop yl]carbamate (Compound 18A) (1.0 eq, 4.00 g, 13.0 mmol) in dichloromethane (50.0 mL), trimethylsilyl trifluoromethanesulfonate (1.1 eq, 2.52 mL, 14.3 mmol) was added dropwise at room temperature. The reaction mixture was stirred at 40° C. for 5 h. After completion, the reaction mixture was quenched with saturated sodium bicarbonate solution and extracted with dichloromethane. The organic layer was dried over sodium sulfate, filtered, and concentrated under high vacuum to get crude. The crude was purified by reverse phase chromatography using 0-30% acetonitrile in water to afford Compound 18B as yellow viscous liquid, Yield: (5.80 g, 70.12%); LCMS m/z 638.2 [M+1]⁺

To a solution of Compound 18B (1.0 eq, 4.80 g, 7.53 mmol) in methanol (40.0 mL), 10% Palladium on carbon (1.60 g) was added and stirred at room temperature under hydrogen atmosphere for 4 h. After completion, the reaction mixture was filtered through syringe filter, filtrate was concentrated and dried to get crude. The crude was triturated with diethyl ether to afford Compound 18C as a pale yellow viscous liquid. Yield: (3.4 g, 80.73%); LCMS m/z 504.37 [M+1]⁺.

A solution of 2,3,4,5,6-pentafluorophenyl 3-(2-{[(benzyloxy)carbonyl]amino}-3-[3-oxo-3-(2,3,4,5,6-pentafluorophenoxy)propoxy]-2-{[3-oxo-3-(2,3,4,5,6-pentafluorophenoxy)propoxy]methyl}propoxy)propanoate (18D) (1.0 eq, 1.20 g, 1.24 mmol) and Compound 18C (3.0 eq, 1.87 g, 3.71 mmol) in N,N-dimethylformamide (30.0 mL) was stirred at room temperature for 1 h. After completion, the reaction mixture was concentrated and dried to get crude. The crude was purified by flash column chromatography using 20% methanol in dichloromethane to afford Compound 18E as pale yellow viscous liquid. Yield: (1.60 g; 67.05%); LCMS m/z 1926.78 [M−1]⁻.

To a solution of Compound 18E (1.0 eq, 1.60 g, 0.830 mmol) in methanol (20 mL) and acetic acid (1.0 mL), 10% Palladium on carbon (250 mg) was added. The reaction mixture was stirred at room temperature under hydrogen atmosphere for 16 h. After completion, the reaction mixture was filtered through celite bed, filtrate was concentrated and dried to afford Compound 18F as pale yellow viscous liquid. Yield: 1.45 g (Crude); LCMS m/z 1794.05 [M+1]⁺.

To a solution of Compound 18F (1.0 eq, 1.45 g, 0.808 mmol) in methanol (10 mL), 25% sodium methanolate solution (8.0 eq, 1.45 mL, 6.47 mmol) was added at 0° C. The reaction mixture was stirred at room temperature for 1 h. After completion reaction, reaction mixture was concentrated and dry to get crude. The crude was diluted with acetonitrile and purified by prep HPLC (30% acetonitrile in water with 0.1% TFA). Fractions containing the desired product were combined and lyophilized to dryness to afford Compound 18G as an off white semi solid. Yield: (0.20 g, 17.4%); LCMS m/z 1415.77 [M+1]⁺.

To a solution of Compound 18G (1.0 eq, 0.090 g, 0.0636 mmol) in dimethyl sulfoxide (1.00 mL), Compound 3E (1.0 eq, 0.030 g, 0.0636 mmol) was added and stirred at room temperature for 16 h. After completion, reaction mixture was diluted with acetonitrile and purified by prep HPLC (42% acetonitrile in water with 0.1% Acetic acid (0-13 min)). Fractions containing the desired product were combined and lyophilized to dryness to afford Compound I-122 as off white solid. Yield: 0.004 g, 3.55%; LC-MS m/z 1769.93 [M+1]⁺; ¹H NMR (400 MHz, DMSO-d₆) δ 7.84 (bs, 3H), 7.73 (bs, 3H), 7.63 (d, J=9.2 Hz, 3H), 7.13 (s, 1H), 4.58-4.54 (m, 4H), 4.47 (bs, 3H), 4.22 (d, J=8.8 Hz, 3H), 3.77-3.67 (m, 12H), 3.53-3.52 (m, 30H), 3.32-3.27 (m, 4H), 3.02 (bs, 14H), 2.29 (t, J=6.0 Hz, 6H), 2.05 (t, J=7.2 Hz, 6H), 1.79 (s, 9H), 1.50-1.41 (m, 18H).

Synthesis of Compound I-124

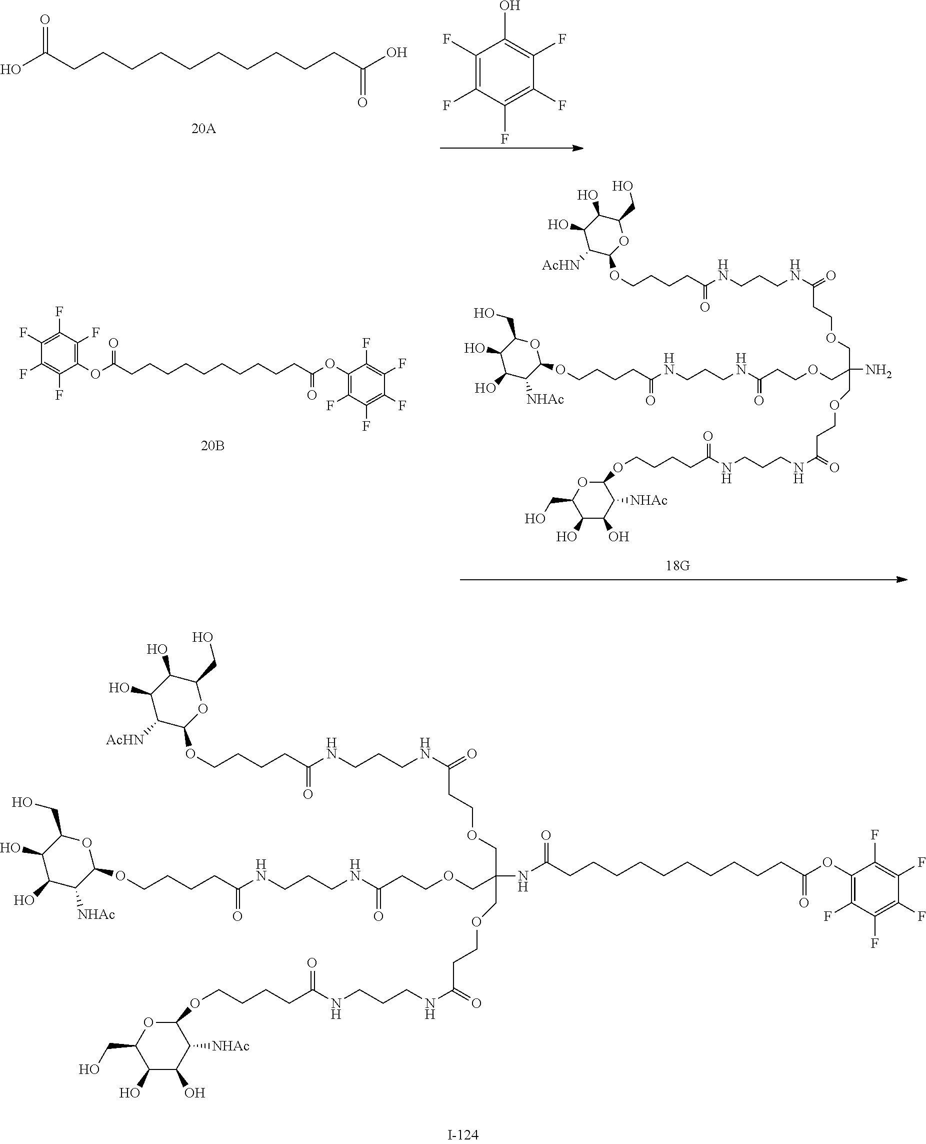

To the solution of dodecanedioic acid (20A) (1.00 g, 4.34 mmol) in ethyl acetate (10.00 mL) at 0° C., pentafluorophenol (1.60 g, 8.68 mmol) and diisopropylmethanediimine (1.91 mL, 13.0 mmol) were added and reaction mixture stirred at room temperature for 1 h. After completion, reaction mixture was filtered through celite bed and filtrate was concentrated under reduced pressure to get crude compound. Crude compound obtained was purified by flash column chromatography on silica gel column using 5% ethyl acetate in hexanes as eluents to afford Compound 20B as off white solid. Yield: 1.00 g (40.95%); LCMS m/z 580.39 [M+18]⁺.

To a solution Compound 18G (45.0 mg, 0.031 mmol) in dimethyl sulfoxide (1.0 mL) was added N,N-diisopropylethylamine (0.016 mL, 0.093 mmol) and Compound 20B (17.9 mg, 0.031 mmol). Reaction mixture was stirred at room temperature for 2 h. After completion, the reaction mixture was purified via preparatory HPLC (40-60% acetonitrile in water with 0.1% trifluoroacetic acid). Fractions containing the desired product were combined and lyophilized to dryness to afford Compound I-124 as an off white solid. Yield: 0.006 g (10.52%); LCMS m/z 1793.94 [M+1]⁺, 897.99 [M/2+1]⁺. ¹H NMR (400 MHz, DMSO-d6) δ 7.83 (t, J=5.6 Hz, 3H), 7.73 (t, J=5.2 Hz, 3H), 7.60 (d, J=9.2 Hz, 3H), 6.99 (s, 1H), 4.57-4.47 (m, 6H), 4.46 (d, J=4.4 Hz, 3H), 4.21 (d. J=8.4 Hz, 3H), 3.70-3.63 (m, 9H), 3.55-3.49 (m, 21H), 3.32-3.28 (m, 4H), 3.02 (t, J=5.6 Hz, 12H), 2.76 (t, J=5.6 Hz, 2H), 2.27 (t, J=6.4 Hz, 6H), 2.03 (t, J=7.2 Hz, 8H), 1.79 (s, 9H), 1.70-1.67 (m, 2H), 1.52-1.41 (m, 20H), 1.23 (bs, 14H).

### 7.3. Preparation of Bifunctional Compounds

Conjugation of Isothiocyanate-Based Ligand-Linker Compounds with Antibodies.

This example provides a general protocol for the conjugation of the isothiocyanate-based ligand-linker compounds (e.g., Compound A) with the primary amines on lysine residues of anti-EGFR antibodies (e.g., matuzumab, cetuximab) and anti-PD-L1 antibodies (e.g., atezolizumab, anti-PD-L1(29E·2A3)). The conjugates thus obtained are listed in Table 2.

The antibody was buffer exchanged into 100 mM sodium bicarbonate buffer pH 9.0 at 5 mg/mL concentration, after which about 30 equivalents of the isothiocyanate-based ligand-linker compound (e.g., Compound A; freshly prepared as 20 mM stock solution in DMSO) was added and incubated overnight at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

Conjugation of Perfluorophenoxy-Based Ligand-Linker Compounds with Antibodies.

This example provides a general protocol for the conjugation of the perfluorophenoxy-based ligand-linker compounds (e.g., Compound I-7) with the primary amines on lysine residues of anti-EGFR antibodies (e.g., matuzumab, cetuximab) and IgG antibodies (e.g., IgG2a-UNLB). The conjugates thus obtained are listed in Table 27.

The antibody was buffer exchanged into 50 mM sodium phosphate buffer pH 8.0 at 5 mg/mL concentration, after which about 22 equivalents of perfluorophenoxy-based ligand-linker compound (e.g., Compound I-7; freshly prepared as 20 mM stock solution in DMSO) was added and incubated for 3 hours at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

Preparation of Antibody Conjugates

Anti-IgG2a conjugates and matuzumab conjugates were generated by adding anti-IgG2a rIgG1 (Southern Biotech, Cat #1155-01) or matuzumab to a solution of PBS buffer pH 7.2 containing 10% DMSO (final antibody concentration 5 mg/mL). Linker (e.g., Compound I-7) was added (at a 5-40× linker:antibody molar ratio), and the reaction was incubated at room temperature for 3 hours. Excess linker was removed by size exclusion chromatography (Superdex 200 Increase, Cat #28990944) and final DAR was determined by LC/MS (Sciex 5600+, Acquity UPLC BEH C4 column, Cat #186004495). The resulting antibody conjugates were used in the experiments below.

Determination of Ligand to Antibody Ratios (i.e., DAR) Values by Mass Spectrometry.

This example provides the method for determining DAR values for the conjugates prepared as described herein. To determine the DAR value, 10 μg of the antibody (unconjugated or conjugated) was treated 2 μL of non-reducing denaturing buffer (10×, New England Biolabs) for 10 minutes at 75° C. The denatured antibody solution was then deglycosylated by adding 1.5 μL of Rapid-PNGase F (New England Biolabs) and incubated for 10 minutes at 50° C. Deglycosylated samples were diluted 50-fold in water and analyzed on a Waters ACQUITY UPLC interfaced to Xevo G2-S QToF mass spectrometer. Deconvoluted masses were obtained using Waters MassLynx 4.2 Software. DAR values were calculated using a weighted average of the peak intensities corresponding to each loading species using the formula below:

DAR=Σ(drug load distribution (%) of each Ab with drug load n)(n)/100

DAR values for the conjugates prepared are shown in Table 27.

Determination of Purity of Bifunctional Antibody Conjugates by SEC.

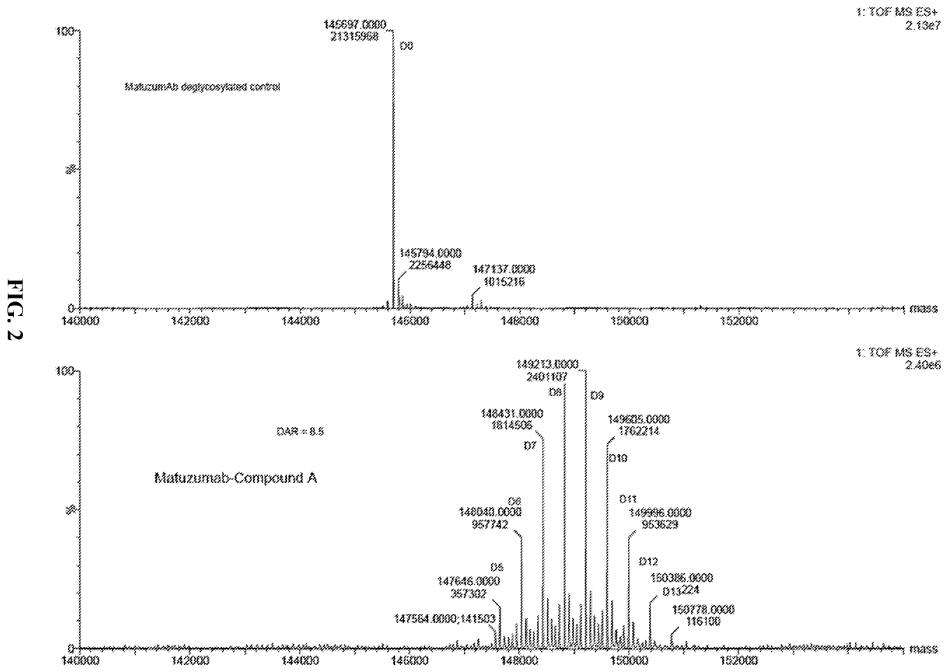

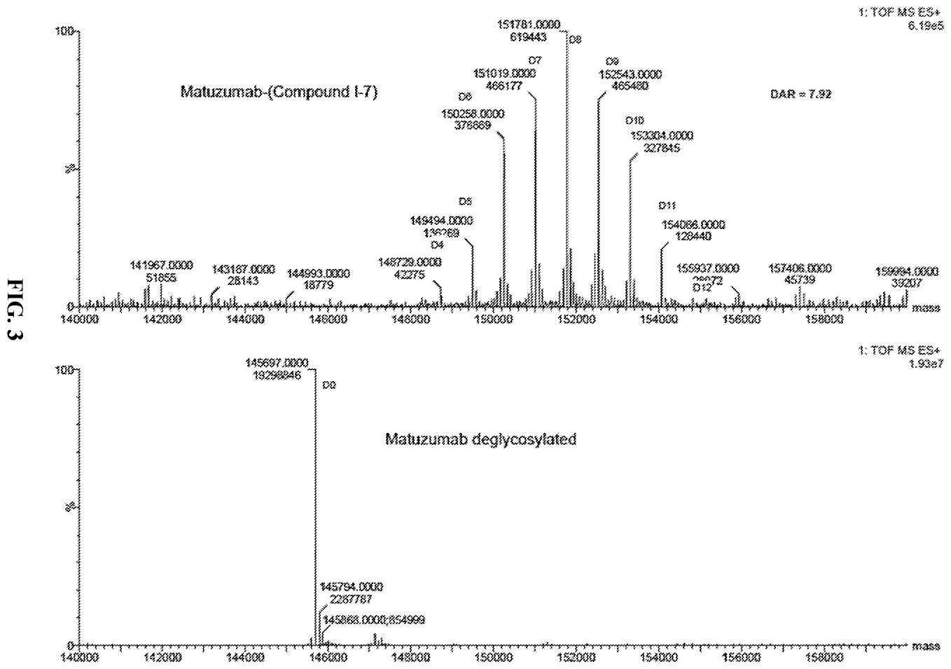

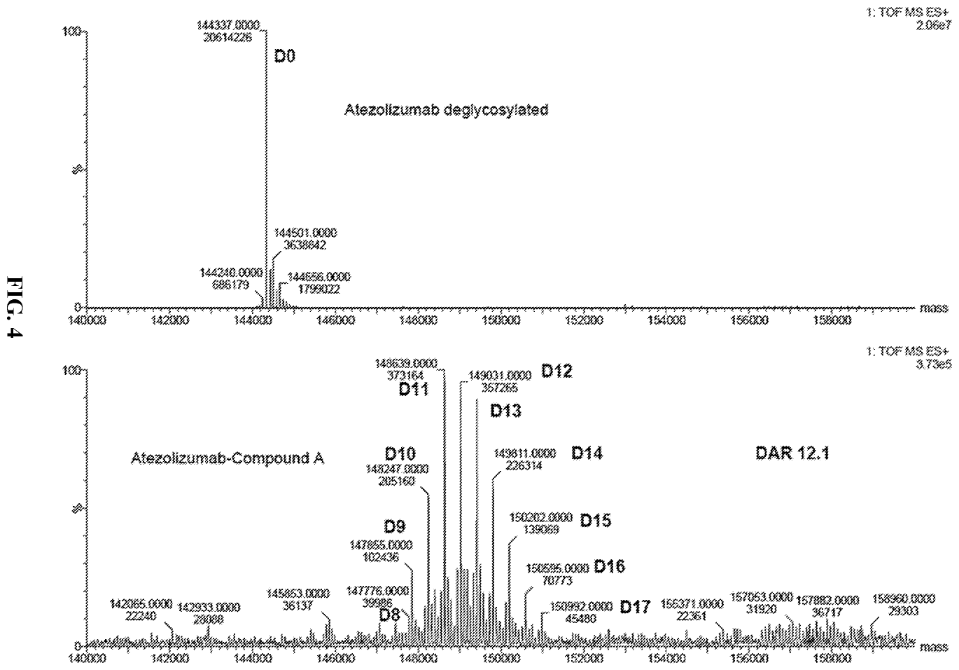

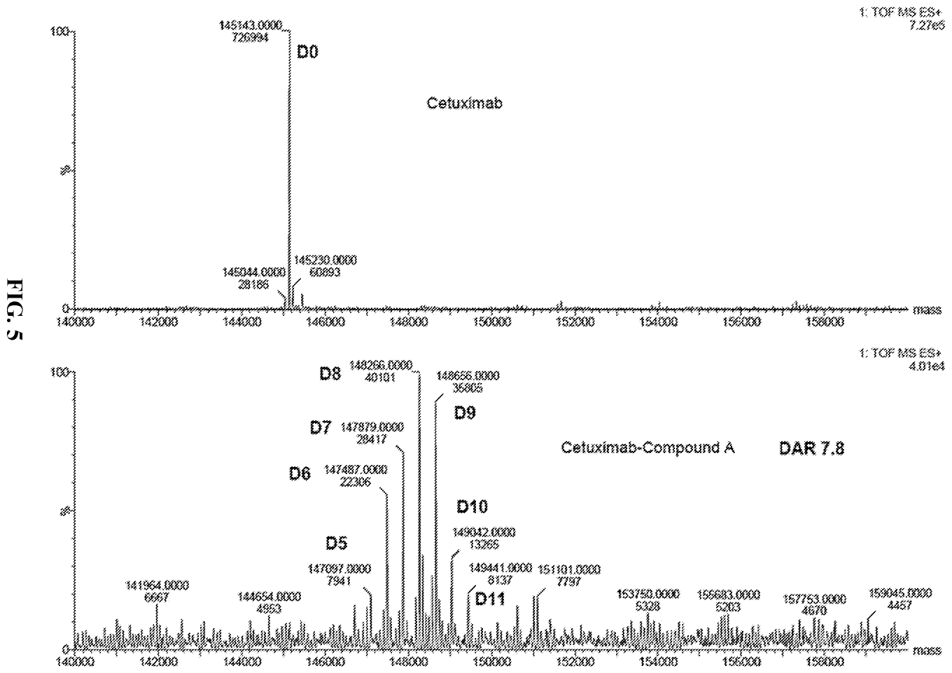

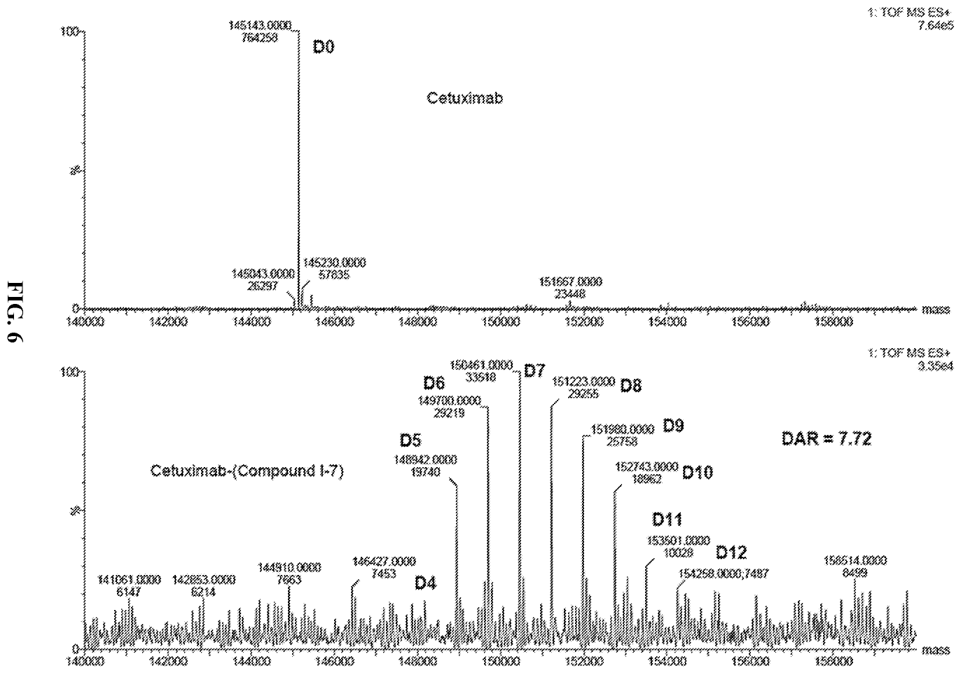

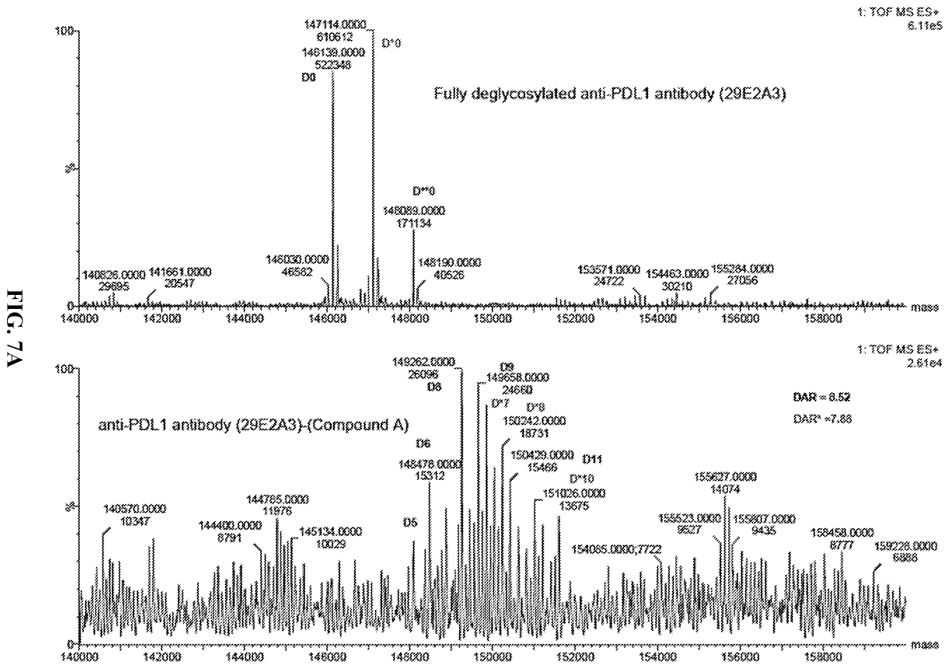

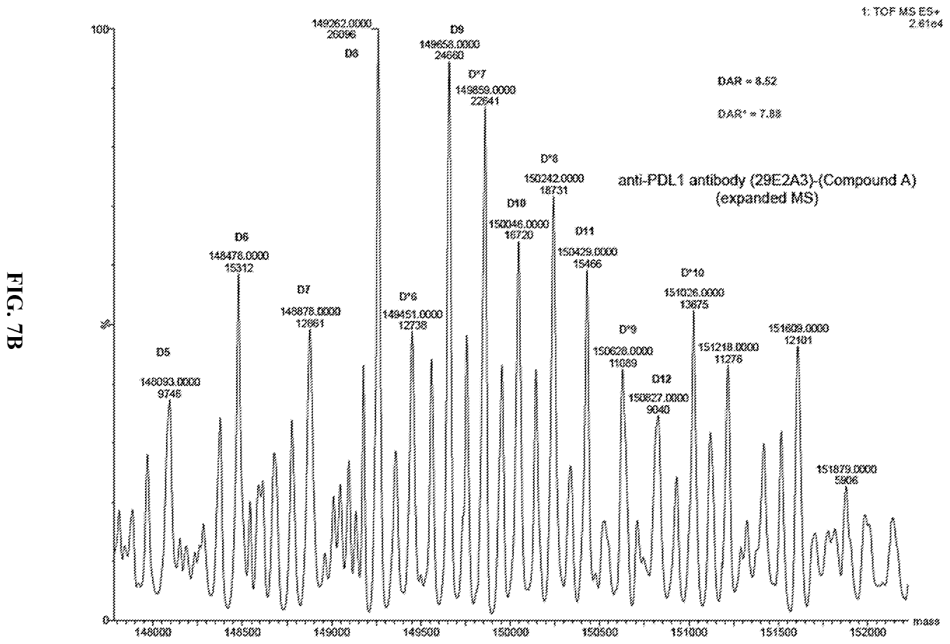

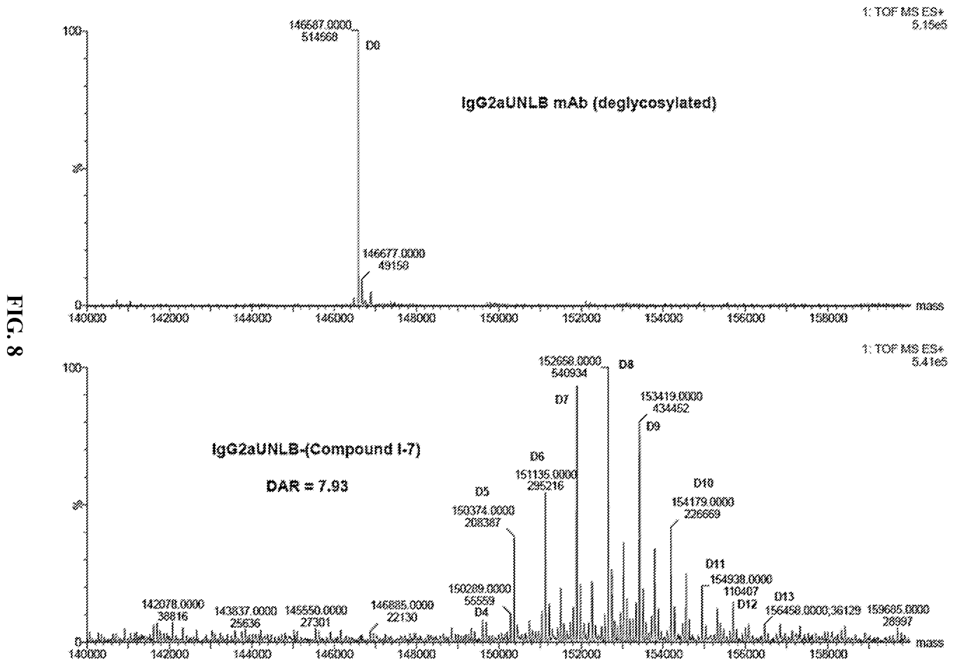

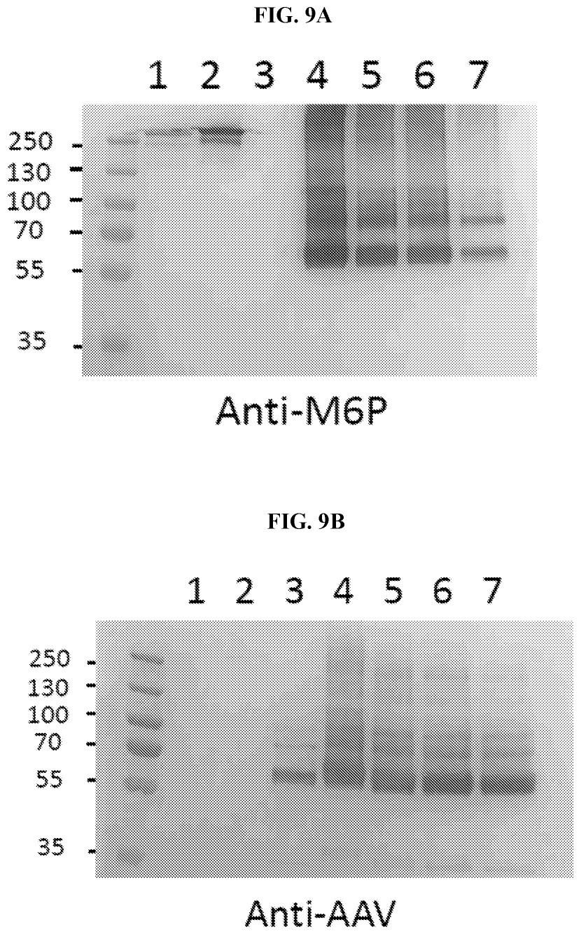

Purity of the conjugates prepared as described herein was determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL was loaded to a TSKgel SuperSW3000 column, at a constant flow rate of 0.35 mL/min. Chromatographs were integrated based on elution time to calculate the purity of monomeric conjugate species. LC-MS data for the conjugates prepared are depicted in FIGS. 2 to 8.

TABLE 27

| | | Ligand-Linker | DAR | Purity |
|---|---|---|---|---|
| Conjugate Name | Antibody | (Compd. No.) | (by MS) | (by SEC) |
| Matuzumab-(Compound A) | Matuzumab | Compound A | 8.5 | >98% |
| Matuzumab-(Compound I-7) | Matuzumab | Compound I-7 | 7.92 | >98% |
| Atezolizumab-(Compound A) | Atezolizumab | Compound A | 12.1 | >96% |
| Cetuximab-(Compound A) | Cetuximab | Compound A | 7.8 | >97% |
| Cetuximab-(Compound I-7) | Cetuximab | Compound I-7 | 7.72 | >98% |
| anti-PD-L1(29E.2A3)-(Compound A) | anti-PD-L1(29E.2A3) | Compound A | 7.9-8.5 | >96% |
| IgG2a-UNLB-(Compound I-7) | IgG2a-UNLB | Compound I-7 | 7.93 | >99% |

Antibody disulfide reduction and ligand-linker conjugation to antibody.

This example provides an exemplary protocol for reduction of the disulfides of the antibodies described herein, and conjugation of the reduced antibodies to the ligand-linker compounds described herein.

Protocol:

### Antibody Disulfide Reduction

A) Dilute antibody to 15 mg/mL (0.1 mM IgG) in PBS, pH 7.4.
B) Prepare a fresh 20 mM (5.7 mg/mL) stock solution of tris(2 carboxyethyl)phosphine (TCEP) in H₂O.
C) Add 25 μL of TCEP stock solution from step B) above to 1 mL of antibody from step A) above (0.5 mM final concentration TCEP).
D) Incubate at 37° C. for 2 hours (check for free thiols using 5,5′-dithiobis-(2-nitrobenzoic acid) (DTNB) test).
E) Aliquot the reduced antibody into 4 tubes (250 μL each).

### Ligand-Linker Conjugation to Antibody

A) Prepare 10 mM stock solution of ligand-linker compound in DMSO (DMA, DMF or CH₃CN are also acceptable).
B) Add 5 equivalents of 12.5 μL stock solution from step A) above to each tube of reduced antibody (0.5 mM final concentration ligand-linker compound stock solution).
C) Incubate overnight at 4° C. for 4 hours at room temperature; check for free thiols using DTNB test.
D) Run analytical hydrophobic interaction chromatography (HIC) to determine DAR and homogeneity.

Expression and Purification of AAV.

Recombinant AAV was expressed in virus production cells using the LV-MAX Lentiviral Production System (Gibco) according to manufacturer protocols.

Recombinant AAV was purified by affinity chromatography using POROS AAVX affinity resin (ThermoFisher) followed by ion-exchange using POROS HQ resin (ThermoFisher) according to manufacturer protocols.

Quantitative assessment of recombinant AAV9 particles was performed by AAV9 Xpress ELISA (Progen) according to manufacturer protocols.

### 7.4. Assessment of Conjugates

Transduction Efficiency of AAV8 and AAV9 Particle Conjugates

Described herein are experiments assessing the transduction efficiency of AAV8 and AAV9 particle conjugate produced herein. The AAV8 or AAV9 particle used here contains a GFP transgene (AAV8-CMV-GFP; Vigene) or a luciferase transgene. The results demonstrate that the addition of the AAV conjugate 1) increases AAV transduction efficiency, 2) overcomes an AAV-resistant cell phenotype and 3) allows the AAV to retain efficient transduction efficiency even in the presence of AAV neutralizing antibodies.

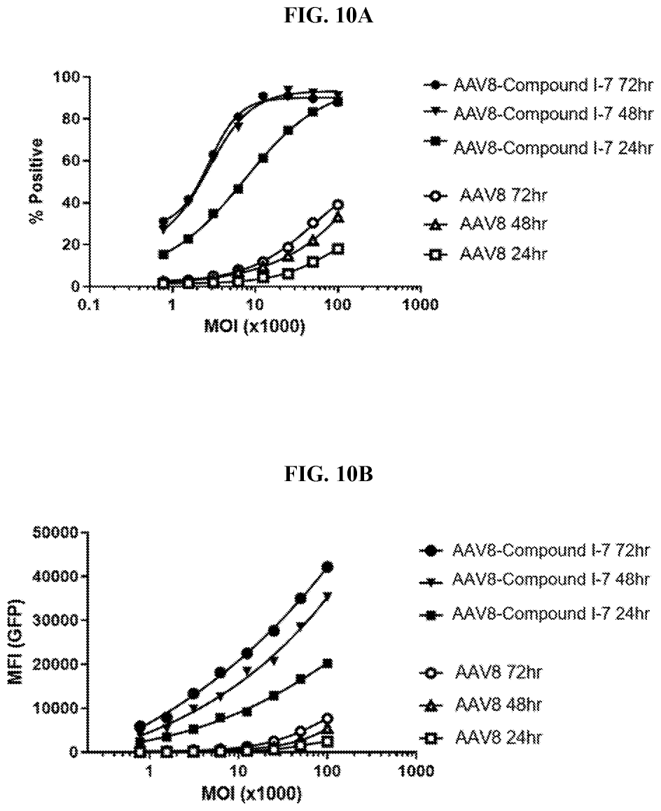

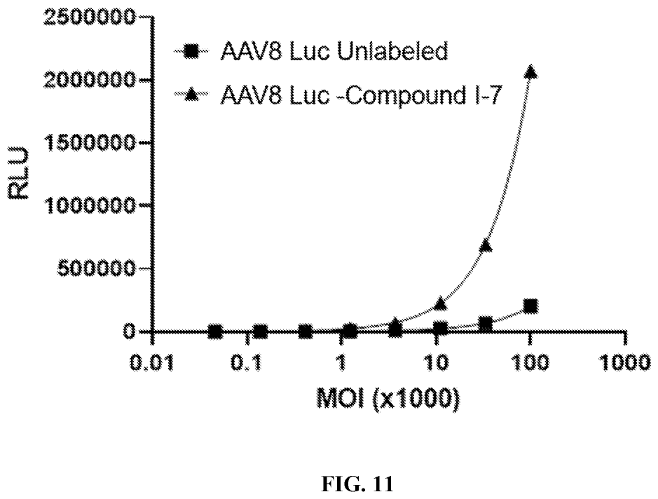

Conjugation increases AAV transduction efficiency. The transduction efficiency of the AAV8 particle conjugate was compared to that of an unconjugated AAV8-CMV-GFP control (Vigene). Transduction efficiencies were measured in human 2V6·11 cells, which express M6PR on their cell surface, and in human Jurkat cells, which have previously been shown to be resistant to AAV8 transduction. As shown in FIGS. 10A and 10B, the transduction efficiency of the AAV8 conjugated to Compound I-7 in 2V6·11 cells after 24 h, 48 h, and 72 h was substantially higher than that of AAV8 alone at 24 h, 48 h and 72 h. Data in FIG. 10A shows the transduction efficiency as a percentage of GFP positive cells, whereas FIG. 10B shows mean fluorescence intensity (MFI) of the GFP positive 2V6·11 cells. Similarly, data shown in FIG. 11 indicate that conjugation increases the transduction efficiency of AAV8 in 2V6·11 cells as measured by luciferase activity.

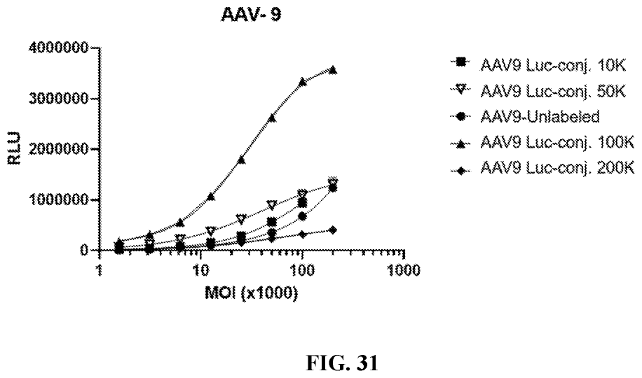

Transduction efficiency of AAV9 particle conjugates was also measured in human 2V6·11 cells and compared to that of unconjugated AAV9-CMV-Luciferase control (“AAV9-Unlabeled”). In these studies, AAV9 containing the luciferase gene was conjugated to Compound I-7 (ITX-16590) (“AAV9 Luc-conj.”). As shown in FIG. 31, increased transduction efficiency was observed for AAV9 conjugated to Compound I-7 compared to unconjugated AAV9 (“AAV9-Unlabeled”) at all but the highest molar ratio of Compound I-7 to AAV9 tested. “10K,” “50K,” “100K,” and “200K” indicate the molar ratio of Compound I-7 to AAV9. The molar ratio of Compound I-7 to AAV9 of 100,000:1 (“100K”) shows the best transduction.

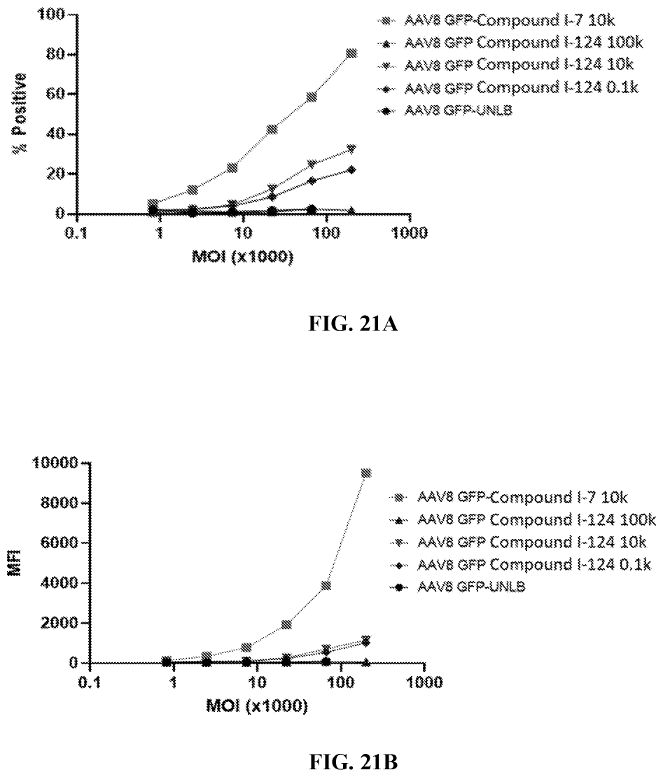

Transduction efficiency of AAV8 particle conjugates was also measured in human HepG2 cells and compared to that of an unconjugated AAV-CMV-GFP control (“AAV8 GFP-UNLB”). HepG2 cells were transduced with increasing multiplicity of infection (MOI) of unconjugated AAV8, AAV8 conjugated to Compound I-7 (ITX-16590), and AAV conjugated to Compound I-124 (ITX-22701) or different molar rations of AAV8 conjugated to Compound I-124 (ITX-22701). As shown in FIGS. 21A and 21B, the transduction efficiency of the AAV8 conjugated to Compound I-7 (ITX-16590) in HepG2 cells was substantially higher than that of unconjugated AAV8. Moderate increase in transduction efficiency was observed for AAV8 conjugated to Compound I-124 (ITX-22701) compared to unconjugated AAV8, with the molar ratio of Compound I-124 (ITX-22701) to AAV8 of 10,000:1 (“10 k”) showing the best transduction. Data in FIG. 21A shows the transduction efficiency as a percentage of GFP positive cells. FIG. 21B shows mean fluorescence intensity (MFI).

Conjugation overcomes an AAV-resistant phenotype. The AAV8 particle conjugate was also able to overcome the AAV transduction resistant phenotype of human Jurkat cells, as demonstrated by the AAV conjugate's ability to efficiently transduce into Jurkat cells. In contrast, AAV8 alone showed no transduction into Jurkat cells at 72h, as expected. See FIGS. 12A and 12B; “unlabeled” indicates AAV8 alone, “10 k” and “25 k” indicate the molar ratio of Compound I-7 to AAV8 (1×10⁴:1 and 2.5×10⁴:1, respectively). FIG. 12A shows the transduction efficiency as a percentage of GFP positive cells, whereas FIG. 12B shows mean fluorescence intensity (MFI).

In performing the experiments described above, 2V6·11 cells (Elab Science) in DMEM (Gibco) with 10% fetal bovine serum (Gibco) were seeded onto 96-well plates (Nunc) at a concentration of 20 k cells/well 24 hours prior to infection. Jurkat cells (ATCC) in RPMI (Gibco) with 10% fetal bovine serum (Gibco) were seeded onto 96-well plates (Nunc) 1 h prior to transduction. HepG2 cells (ATCC) in EMEM (Gibco) with 10% fetal bovine serum (Gibco) were seeded onto 96-well plates (Nunc) 24 hours prior to infection.

In transducing the 2V6·11 cells, the AAV8 particle conjugate and an unconjugated AAV8-GFP control (Vigene) at a multiplicity of infection (MOI) of 0.8, 1.6, 3.1, 6.2, 12.5, 25, 50 or 100×10³ were added to cells which were then incubated at 37° C. for 72 hrs. In transducing 2V6·11 cells with AAV9 particle conjugate, the AAV9 particle conjugate and an unconjugated AAV9-Luciferase at a MOI of 2-200×10³ were added to cells which were then incubated at 37° C. for 24 hrs. In transducing the HepG2 cells, the AAV8 particle conjugates and unconjugated AAV8 particle at an MOT of 0.7 to 300×10³ were added to cells which were then incubated at 37° C. for 72 hrs.

After the incubation period, the medium was aspirated, the cells were washed with PBS and analyzed for GFP expression via flow cytometry on BioRad ZES Cell Analyzer (BioRad) or analyzed for luciferase levels using a luciferase assay kit (Promega) following manufacture's protocol and using a luminometer.

Conjugation allows the AAV to retain transduction efficiency in the presence of neutralizing antibody. ADK8 is an AAV8 neutralizing antibody (NAb) that inhibits transduction efficiency of AAV8, including transduction efficiency of AAV8 into human 2V6·11 cells and Jurkat cells. See FIGS. 13A-13D.

Briefly, transductions were performed in the presence or absence of 2 ng/ml, 4 ng/ml, 8 ng/ml, or 16 ng/ml ADK8 neutralizing antibody at a MOI of 5×10⁴ of either the particle conjugate or the AAV8 particle alone. A study utilizing ADK8 demonstrates that the AAV8 particle conjugate can efficiently transduce human 2V6·11 cells (FIGS. 13A and 13B) and human Jurkat cells (FIGS. 13C and 13D) even in the presence of the AAV8 neutralizing antibody. FIGS. 13A and 13C show the transduction efficiency as a percentage of GFP positive cells, whereas FIGS. 13B and 13D show mean fluorescence intensity (MFI). “10 k” and “25 k” indicate the ratio of Compound I-7 to AAV8 (1×10⁴:1 and 2.5×10⁴, respectively).

As shown in FIG. 13A-13B, the presence of the ADK8 neutralizing antibody did not affect the transduction efficiency of the AAV8 particle conjugate, but, in contrast, substantially decreased the transduction efficiency of AAV8 alone.

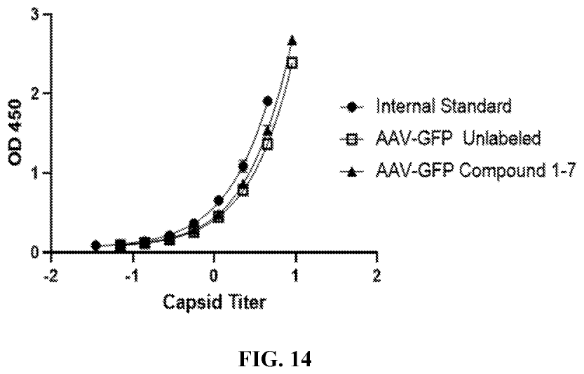

Interestingly, the AAV8 particle conjugate retains its transduction efficiency even though results indicate that the AAV8 particle conjugate also retains an ability to bind the AAV8 neutralizing antibody, ADK8. Briefly, ADK8 Nab binding to GFP-AAV8 alone and to the AAV8 particle conjugate were measured using an ELISA kit (Progen) according to manufacturer's instructions. The data shown in FIG. 14 indicates that the AAV8 particle-conjugate retains an ability to bind to ADK8 similar to that of AAV8 alone. Thus, the increased transduction efficiency observed with the AAV8-Compound I-7 conjugate is not due to lack of binding to neutralizing antibodies.

In performing these experiments, human 2V6·11 cells (Elab Science) in DMEM (Gibco) with 10% fetal bovine serum (Gibco) or Jurkat cells (ATCC) in RPMI (Gibco) were seeded onto 96-well plates (Nunc) 24 h prior to infection. Unconjugated AAV8-CMV-GFP (Vigene) or conjugated AAV8-CMV-GFP at a multiplicity of infection (MOI) of 5×10⁴ were preincubated with 0, 2, 4, 8, or 16 ng of ADK8 antibody (Origene) for 30 min at 37° C. and then added to cells. After 72 hours, the medium was aspirated, cells were washed with PBS, and DMEM with FCS was added. After removing culture supernatant, the cells were washed with PBS and analyzed for GFP expression via flow cytometry on BioRad ZES Cell Analyzer (BioRad) and GFP expression analysis was performed.

Increased Transduction Efficiency is Mediated by Mannose 6 Phosphate Receptor (M6PR) Expression

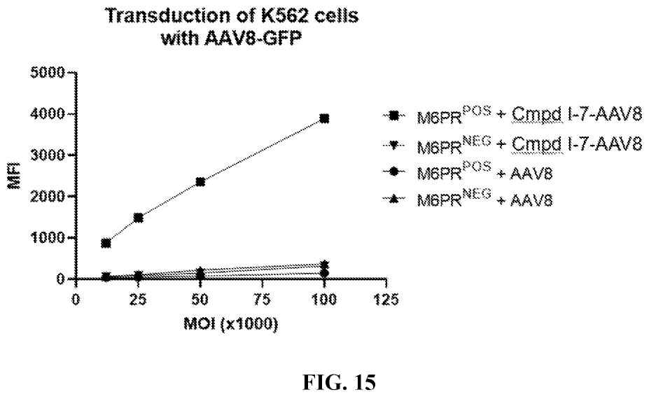

Increased transduction efficiency of AAV8 particle conjugate is dependent on the cell surface expression of M6PR. Utilizing a human K562 cell line that either expresses M6PR on the cell surface (M6PR^{POS}) and a companion K562 cell line where M6PR has been deleted (M6PR^{NULL}) in transduction studies demonstrates the increased transduction efficiency of the AAV8 particle only observed in the M6PR^{PO} K562 cell line. See FIG. 15.

Briefly, transductions were performed at an MOI of 2.5×10⁴ of either the particle conjugate or the AAV8 particle alone in K562 expressing M6PR on the cell surface (M6PR^{PRO}) or K562 that have bear a null mutation in M6PR and thus do not express M6PR on the cell surface (M6PR^{NEG}). FIG. 15 shows the transduction efficiency as mean fluorescent intensity of GFP positive cells. As shown in FIG. 15, the increased transduction efficiency up the AAV8 particle conjugate was only observed in K562 cells expressing M6PR on the cell surface (M6PR^{POS}). The transduction efficiency of the AAV8 particle conjugate was similar to the AAV8 particle alone in cell that did not express M6PR on the cell surface (M6PR^{NEG}). These results demonstrate the dependence of this receptor to facilitate increased transaction efficiency of the AAV8 particle conjugate.

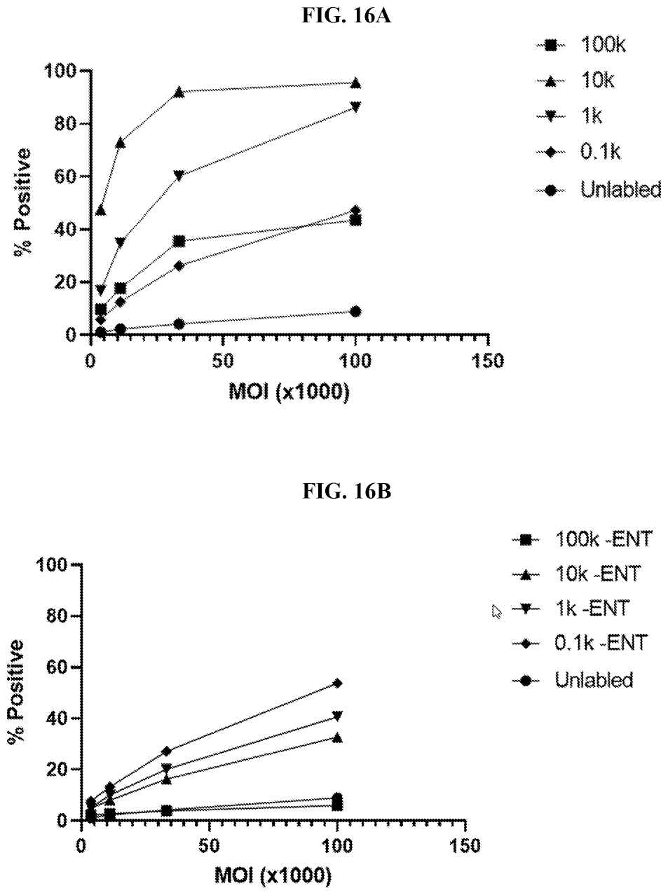

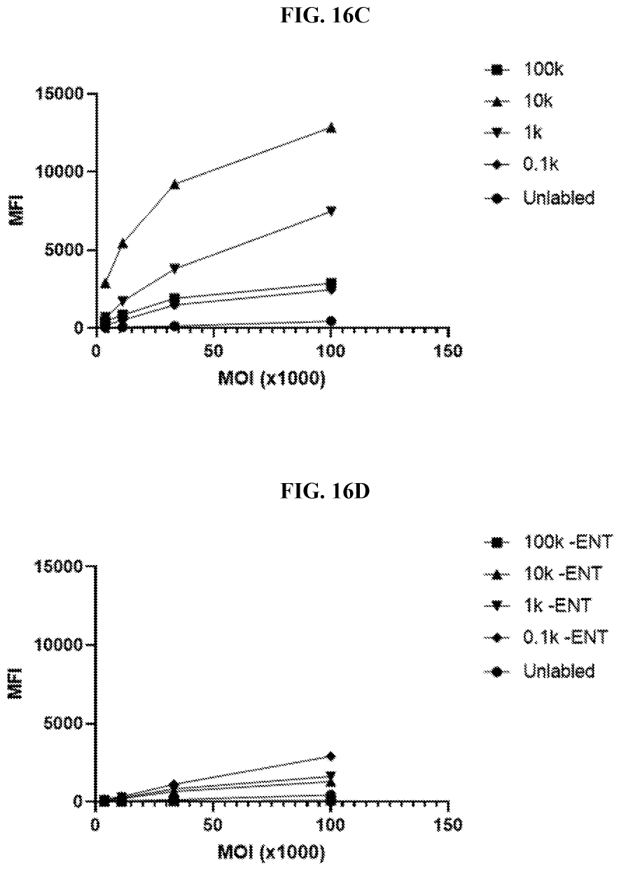

Increased transduction efficiency of AAV8 particle conjugate is not observed when conjugated to inactive enantiomer of the Compound I-7. See FIG. 16. Briefly, transductions were performed in human 2V6·11 cells at an MOI of 10, 3, 1, and 0.3×10⁴ of either the particle conjugated to Compound I-7 or capsid conjugated to the inactive enantiomer of Compound I-7. FIGS. 16A and 16B show the transduction efficiency as a percentage of GFP positive cells, whereas FIGS. 16C and 16D shows mean fluorescence intensity (MFI). “10 k” and “100 k” indicate the ratio of Compound I-7 to AAV8 (1×10⁴:1 and 2.5×10⁴, respectively). AAV8 capsid conjugated to Compound I-7 (FIGS. 16A and 16C) showed transduction efficiency when compared to AAV8 capsid conjugated to the inactive enantiomer of Compound I-7 (FIGS. 16B and 16D).

Increased Transduction Efficiency is Mediated by Mannose 6 Phosphate Receptor (M6PR) Binding

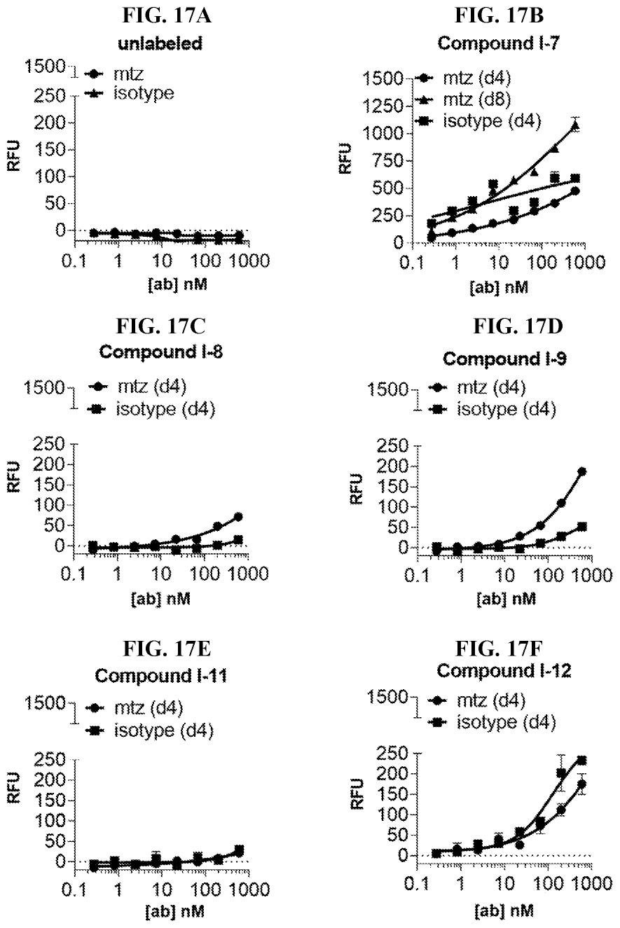

Binding affinity of matuzumab conjugated to ligand-linkers for M6PR was determined by ELISA using the following protocol: Nunc black solid bottom MaxiSorp plates were allowed to incubate overnight at 4° C. coated with 1 μg/mL of recombinant human CI-M6PR protein (R&D, 6418-GR-050) in 50 μL PBS. The next day, coating was decanted and plates were washed 3× with PBS. Wells were blocked with 350 μL of 3% BSA-PBS for 1 hour at room temperature. Blocking solution was removed and matuzumab conjugates (matuzumab-Compound I-7(d4), matuzumab-Compound I-7(d8), matuzumab-Compound I-8 (d4), matuzumab-Compound I-9(d4), matuzumab-Compound I-11 (d4) and matuzumab-Compound I-12 (d4)) and their respective isotype controls (human IgG (bioxcell, BP0297) conjugated to the ligand-linker compounds being tested) were diluted in 3% BSA-PBS. 50 μL of diluted conjugates were added to the plate and allowed to incubate at room temperature for 2 hours. After incubation, solutions in plate were decanted and washed with 350 μL of 0.05% PBS-Tween20 three times, drying the plate each wash on a clean paper towel. 50 μL of peroxidase AffiniPure Mouse Anti-Human IgG (Jackson Immuno, 209-035-088) diluted in 3% BSA-PBS to 0.2 μg/mL was added to the plate and allowed to incubate for 1 hour at room temperature in the dark. After incubation, solutions in plate were decanted and washed with 350 μL of 0.05% PBS-Tween20 3 times, drying the plate each wash on a clean paper towel. QuantaBlu fluorogenic peroxidase substrate (ThermoFisher, 15169) was prepared per manufacturer's suggestions and equilibrated to room temperature. 50 μL of QuantaBlu solution was added to wells and allowed to incubate for 5-10 minutes at room temperature. After incubation, plates were read on a Perkin Elmer EnVision using photometric 340 and Umbelliferone 460 filter sets for excitation and emission, respectively. Data analysis and non-linear curve-fitting was performed using GraphPad Prism. FIGS. 17A-17E shows the binding affinities of the conjugates tested for M6PR, with Compound I-7d8 and Compound I-11d4 displaying the highest and lowest binding affinity, respectively.

Serum Pharmacokinetic (PK) Analysis for rIgG1 Antibody Conjugates of Varying Binding Affinities

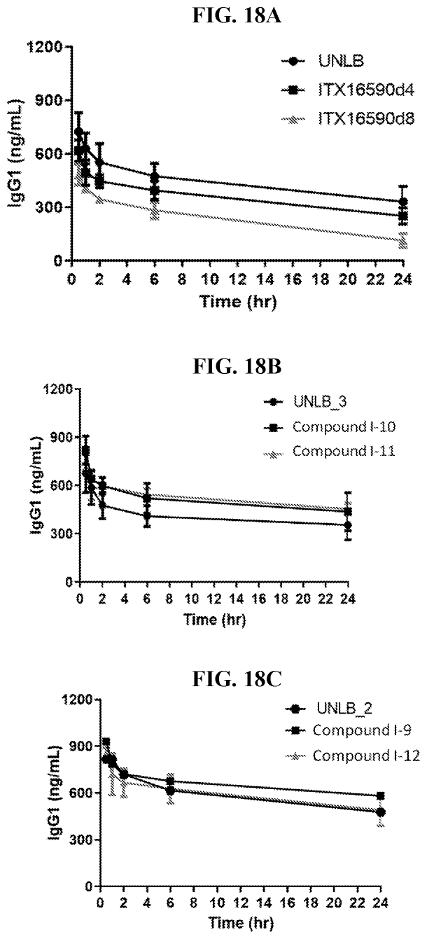

A pharmacokinetic analysis of the rIgG1 (anti-IgG2a) antibody conjugates described in the previous example was performed in mice. In particular, C57B6 mice were intravenously administered each rIgG1 antibody conjugate at 10 μg/mouse (5 mice per group). Blood was collected at 0.5, 1, 2, 6, and 24 hours and serum rIgG1 was analyzed using an ELISA kit (Abcam) according to the manufacturer's instructions. Samples were run across 3 different plates with unconjugated rIgG1 controls (UNLB-anti-IgG2a rIgG1) included on all 3 plates. FIGS. 18A-18C show the serum levels of aIgG2a conjugated to Compound I-7 (dar8) and (dar4) (FIG. 18A), aIgG2a conjugated to Compound I-10 and aIgG2a conjugated to Compound I-11 (FIG. 18B), and aIgG2a conjugated to Compound I-9 and aIgG2a conjugated to Compound I-12 (FIG. 18C) over time.

As shown in FIGS. 18A-18C, the results demonstrate that conjugates of ligand linkers such as Compounds I-9, I-10, I-11, and I-12 which have weaker binding affinity to M6PR compared to Compound I-7 exhibit longer half-life, and therefore may be useful for tuning the pharmacokinetic properties of the conjugate.

Conjugates of Varying Binding Affinities Mediate Uptake of IgG2a into Cells Over Time

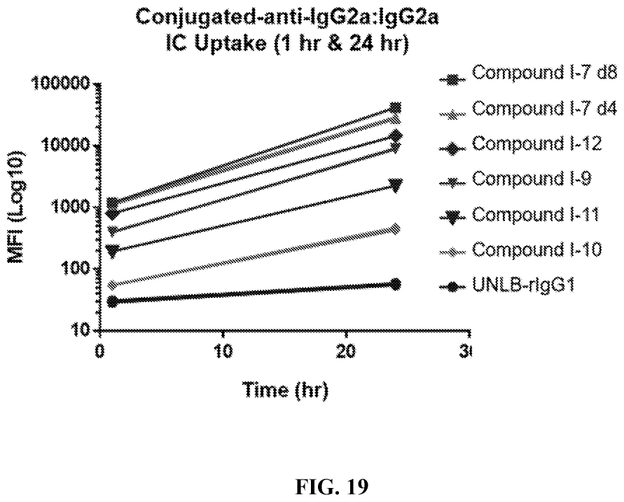

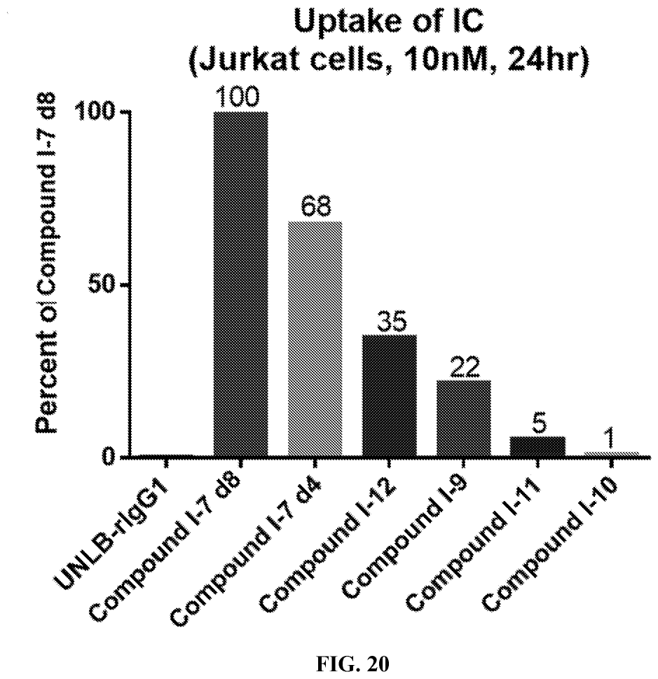

The anti-IgG2a conjugates were bound to IgG2a-Alexa488, as follows: equal molar ratios of anti-IgG2a and IgG2a-Alexa488 were added in tissue culture media for 30 minutes at room temperature. The resulting anti-IgG2a:IgG2a antibody-Alexa488 compositions were added to human Jurkat cells (100 k cells/50 ul per well, n=2), and Alexa488 fluorescence levels were measured (via Alexa488 measurement) at 1 hour and 24 hours by flow cytometry. Because Alexa488 accumulates in cells, this presents a way to measure total intracellular uptake by cells over time. FIG. 19 shows the intracellular levels of aIgG2a conjugates Compound I-7 (dar8) and (dar4), Compound I-10, Compound I-11, Compound I-9, and Compound I-12 at 1 h and 24 h. FIG. 20 shows the intracellular uptake of the tested conjugates into Jurkat cells at 10 nM after 24 hours as a percentage of the uptake of aIgG2a conjugate-Compound I-7d8. These data indicate that conjugates of ligand linkers with weaker binding affinity to M6PR than Compound I-7, such as Compounds 1-9, 1-10, 1-11 and 1-12, still exhibit sufficiently robust uptake, and may therefore be useful for tuning the pharmacokinetic properties of the conjugate, while still capable of mediating uptake.

Transduction Efficiency and Transgene Expression of AAV8 Particle Conjugates in the Presence of Anti-AAV8 Neutralizing Antibodies

In order to evaluate whether AAV8 particle conjugates can express the luciferase transgene contained in the viral particle in the presence of AAV8 neutralizing antibodies, human 2v6.11 cells were transduced with AAV8-CMV-Luciferase conjugated to Compound I-7 (ITX-16590) or unconjugated AAV8-CMV-Luciferase in increasing dilutions of pooled human serum.

Briefly, 2V6·11 cells (Elab Science) in DMEM (Gibco) with 10% fetal bovine serum (Gibco) were seeded onto 96-well plates (Nunc) 24 hours prior to infection. Unconjugated AAV8-CMV-Luciferase or conjugated-AAV8-CMV-Luciferase at a multiplicity of infection (MOI) of 5×10⁴ were mixed with three-fold dilutions (ranging from 1 to 1/3000) of a pool of human serum known to contain anti-AAV8 neutralizing antibodies and were incubated for 30 minutes at 37° C. Following incubation, the AAV8/serum mixtures were added to the cells. The cells were incubated with the AAV8/serum mixtures, the medium was then aspirated and the cells were washed with PBS. The cells were analyzed for luciferase expression using a luciferase assay kit (Promega) and a luminometer.

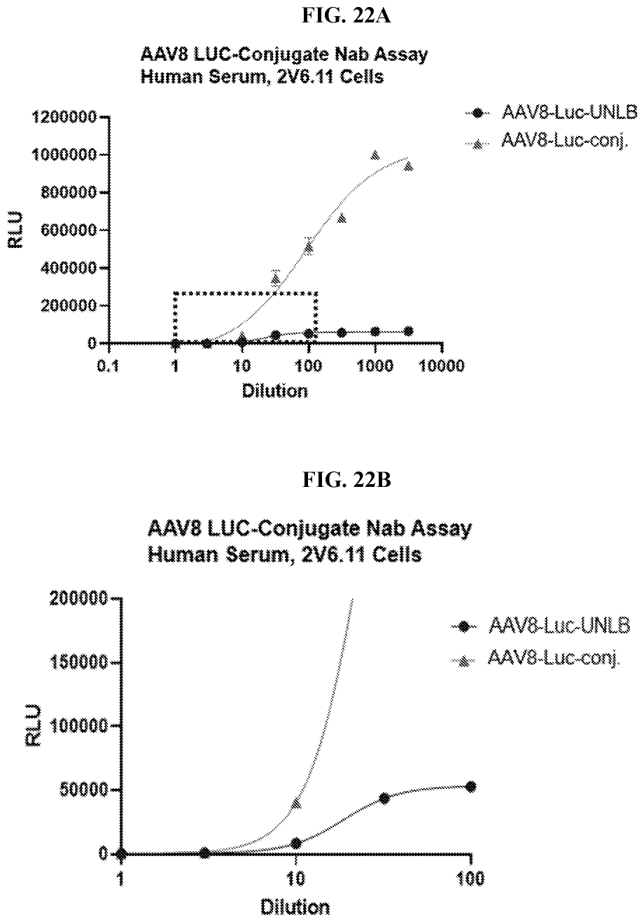

As shown in FIGS. 22A and 22B, robust luciferase expression was observed from conjugated AAV8 even at very low dilutions of human serum. Although some neutralization was observed, considerable transgene expression was shown even in the presence of AAV8 neutralizing antibodies.

AAV8 GalNAc Conjugates Demonstrate Robust Transgene Expression and Restricted Tropism to Liver In Vivo

In order to assess transgene expression of AAV8 particle conjugates in vivo, mice were treated with conjugated and unconjugated AAV8 containing the luciferase transgene and subjected to bioluminescence imaging. Conjugated AAV8 included, separately, AAV8 particles conjugated to N-Acetylgalactosamine (GalNAc) and GalNAc enantiomer.

Briefly, BALB/c mice were housed in vivarium under SPF2 conditions with 12 hour light cycles and ad libitum access to food and water. Mice were injected intravenously with doses of 1×10¹¹, 3×10¹⁰, or 1×10¹⁰ vector genome (vg) per mouse of GalNAc-conjugated AAV8-luciferase, enantiomer GalNAc-conjugated AAV8-luciferase, or unconjugated AAV8-luciferase. Tissue-specific expression of luciferase in each animal was assessed via in vivo bioluminescence imaging on days 3, 7, 14, and 21 post-dosing.

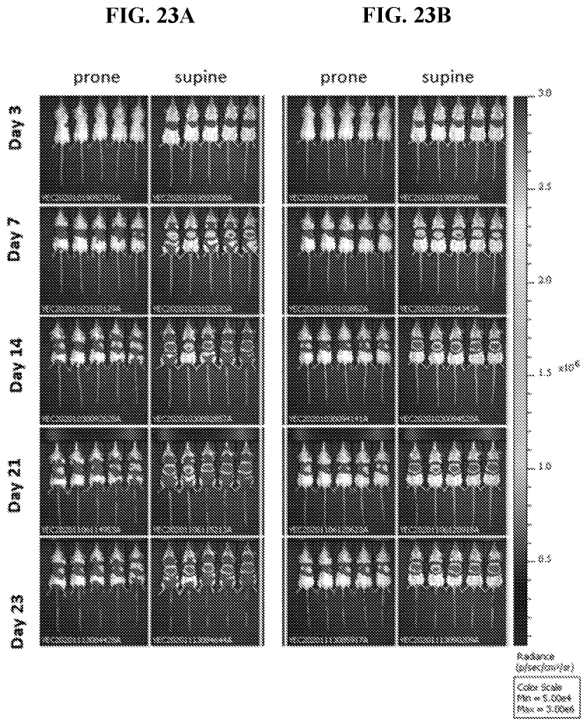

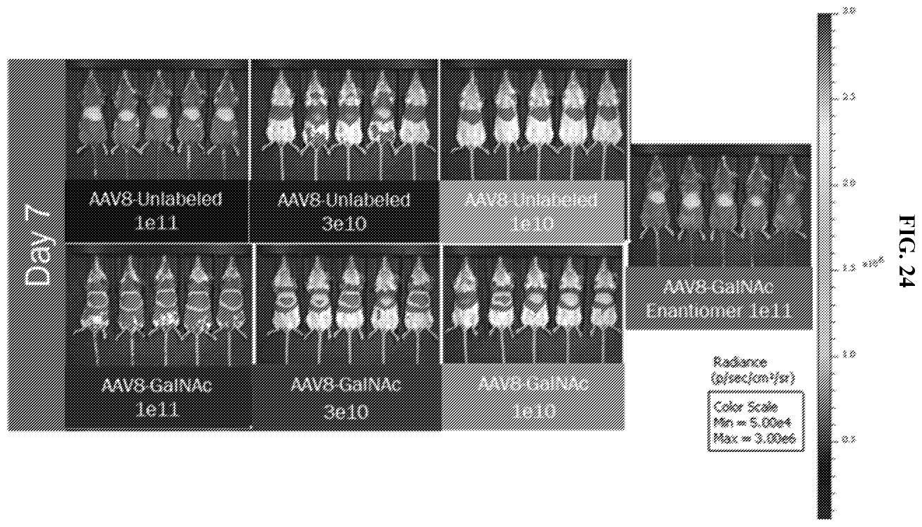

Results in FIGS. 23A and 23B show luciferase expression in representative animals at 3, 7, 14, 21, and 24 days post-dosing with unconjugated AAV-luciferase (FIG. 23A) and GalNAc-conjugated AAV8-luciferase (FIG. 23B). Robust luciferase expression was seen throughout the animals dosed with unconjugated AAV8-luciferase within 7 days (FIG. 23A). By contrast, animals dosed with GalNAc-conjugated AAV8-luciferase show luciferase expression primarily in the liver, demonstrating restricted liver tropism of GalNAc-conjugated AAV8.

As shown in FIG. 24 (right panel), mice dosed with AAV8-luciferase conjugated to the enantiomer of GalNAc show luciferase expression throughout the body, similar to mice dosed with the same amount of unconjugated AAV8-luciferase (FIG. 24, top left). These results suggest that the restricted liver tropism that is observed of GalNAc-conjugated AAV8 is mediated by the asialoglycoprotein receptor (ASGPR) since GalNAc but not its enantiomer can bind ASGPR.

Transduction Efficiency and Transgene Expression of AAV8 Particle Conjugates in Human Primary Cell Lines

In order to assess transduction efficiency of AAV8 particle conjugates in human primary cell lines, cells were transduced with AAV8-CMV-Luciferase conjugated to Compound I-7 (ITX-16590) or unconjugated AAV-CMV-Luciferase and then analyzed for expression of luciferase.

Briefly, primary human fibroblasts (Cell Biologics), primary human endothelial cells (Cell Biologics), primary human hepatocytes (Sigma), and primary human skeletal muscle cells (Cell Biologics) were cultured according to according to supplier's protocols and were seeded 24 hours prior to infection. Unconjugated AAV8-CMV-Luciferase or conjugated-AAV8-CMV-Luciferase at a multiplicity of infection (MOI) of 2-200×10³ were added to cells which were then incubated at 37° C. for 24 hours. Following incubation, the cells were washed with PBS and analyzed for luciferase levels using a luciferase assay kit (Promega) and a luminometer.

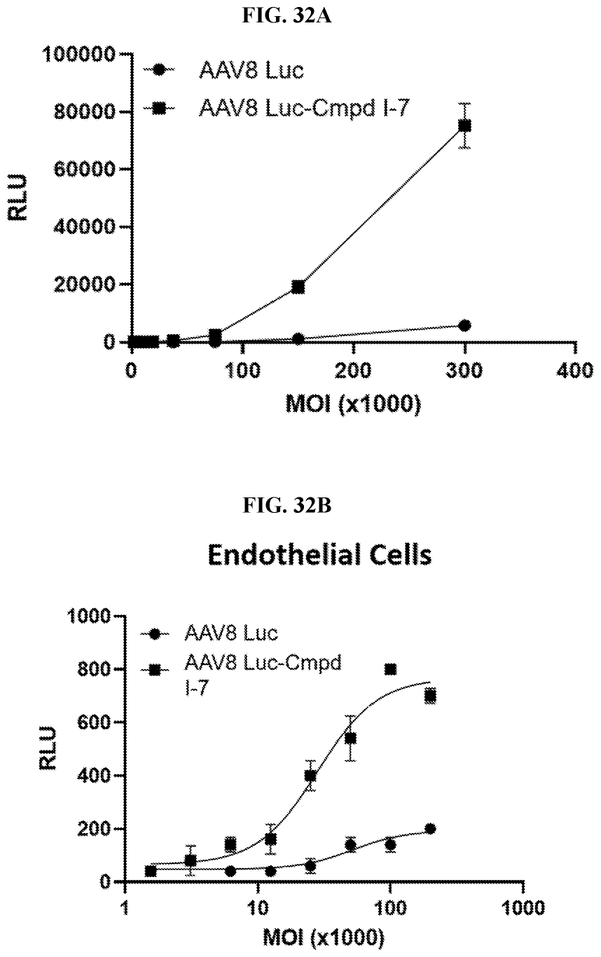

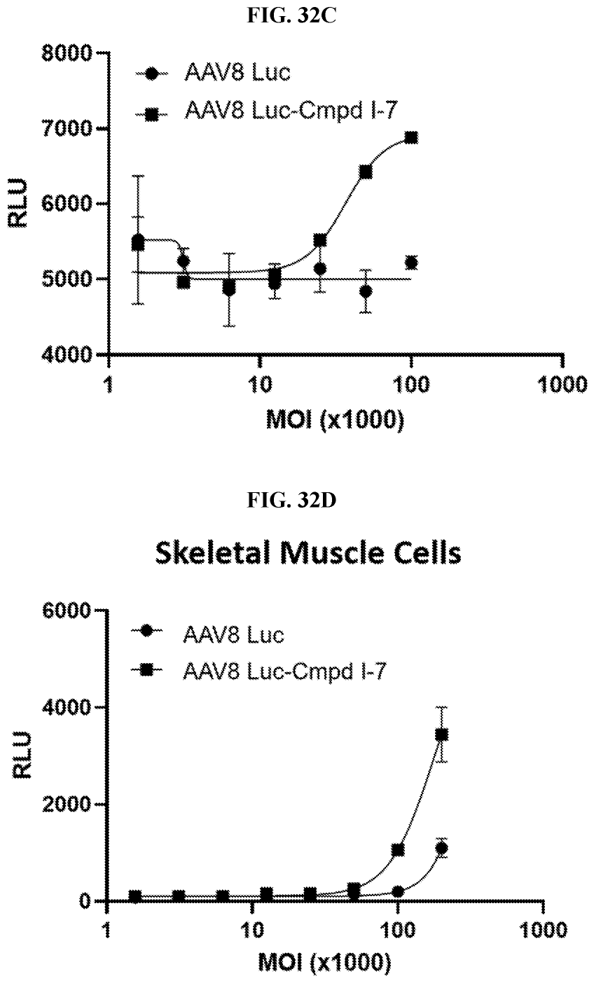

Results in FIGS. 32A-32D show improved transduction efficiency of the AAV8 conjugates compared to unconjugated AAV8 in all four human primary cell lines tested. Transduction with AAV8 Luciferase conjugated to Compound I-7 (“AAV8 Luc-Cmpd I-7”) resulted in increased transgene expression compared to unconjugated AAV8 Luciferase (“AAV8 Luc”) in primary human fibroblasts (FIG. 32A), primary human endothelial cells (FIG. 32B), primary human hepatocytes (FIG. 32C), and primary human skeletal muscle cells (FIG. 32D). Luciferase expression was particularly high in fibroblasts (FIG. 32A) and hepatocytes (FIG. 32C) transduced with AAV8 particle conjugates, demonstrating that conjugation improves transduction efficiency and transgene expression especially well in these human cell types.

Transduction Efficiency of AAV8-Luciferase Linked to a Cell Surface Binding Moiety Via a Bridging Antibody

In order to assess transduction efficiency of luciferase-encoding AAV8 particles linked to a cell surface binding moiety via an antibody, cells were transduced with AAV8-luciferase and either unconjugated anti-AAV8 antibody (ADK8) or ADK8 conjugated to Compound I-7 (ITX-16590) and then analyzed for luciferase expression.

In performing these experiments, human 2V6·11 cells (Elab Science) in DMEM (Gibco) with 10% fetal bovine serum (Gibco) were seeded onto 96-well plates (Nunc) 24 hours prior to infection. AAV8-CMV-Luciferase (Vector Biolabs) at a multiplicity of infection (MOI) of 5×10⁴ were preincubated with 1.56-200 ng of ADK8 antibody or Compound I-7 (ITX-16590) conjugated ADK8 antibody for 30 minutes at 37° C. and then added to cells. After 24 hours, the cells were washed with PBS and analyzed for luciferase levels using a luciferase assay kit (Promega) and a luminometer.

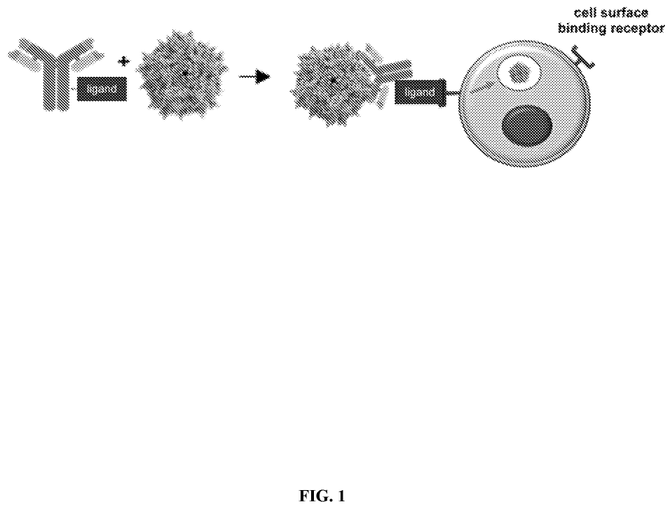

A schematic of the interaction between the AAV8 particle and the ADK8 antibody conjugated to ligand (e.g., Compound I-7) is shown in FIG. 1. Results shown in FIG. 25 demonstrate that in the presence of the Compound I-7-conjugated ADK8 antibody transduction efficiency of AAV8-luciferase is increased compared to transduction of AAV8-luciferase alone. Increased levels of ADK8 resulted in lower transduction, possibly due to ADK8 functioning as a neutralizing antibody.

### 7.5. Preparation and Assessment of Exemplary IGF-2 Polypeptide Containing Bifunctional Compounds

Preparation of Omalizumab-IGF2 Bifunctional Compound

A bifunctional compound (1) was prepared via conjugation of omalizumab (anti-IgE antibody) and IGF-2 polypeptide using the bivalent linker 6-maleimidocaproic acid sulfo-NHS. Recombinant IGF-2 polypeptide was obtained from R&D Systems (Ala25-Glu91), and conjugated with the NHS ester of the bivalent linker, e.g., at the N-terminal amine group and/or the sidechain amine group of the lysine residue of IGF-2. The linker modified IGF-2 polypeptide was then conjugated with the antibody. Omalizumab antibody having site-specific mutation L443C was used for conjugation of the cysteine sidechain group to the maleimide group of the linker.

The purity of the conjugate is determined through size exclusion high performance liquid chromatography (SEC-HPLC). The ratio of IGF-2 polypeptide to antibody in the conjugates is determined using mass spectrometry.

For comparison, conjugates of the omalizumab antibody with alternative cell surface receptor ligands were prepared using similar methods, including a mannose-6-phosphate-ligand (M6P)-linker precursor or a M6Pn ligand-linker precursor (see e.g., Compound I-7).

Preparation of Fluorescently Labelled Compounds

The omalizumab antibody conjugates are labelled with Alexa Fluor 488 (AF488). Protein Labeling Kit (Invitrogen) per the manufacturer's protocol. In brief, antibodies to be labeled are diluted to 2 mg/mL in PBS to a total volume of 500 μL. A 15 DOL (degree of labeling) is used for the conjugation with the fluorophore. Free dye is removed by pre-wetting an Amicon 30 kDa filter with PBS. After incubation, the conjugation reaction is then added to the filter and spun at high speed for 10 minutes. Retained solution is then resuspended in PBS to a final volume of 1 mL and stored at 4° C.

Assessment of Exemplary Bifunctional Compounds

In Vitro Cell Uptake Assay

Omalizumab was conjugated to M6P, M6Pn, or IGF-2 polypeptide as described above or left unconjugated (UNLB). The omalizumab compositions were then fluorescently labelled with Alexa 488 fluorescent dye as described above.

Uptake of the omalizumab-AF488 compositions was evaluated in Jurkat (human), C2C12 (mouse) and primary mouse fibroblasts cells. Cells were incubated for 1 hour with the compositions and then cellular uptake was assessed by measuring mean fluorescent intensity (MFI) of cells using flow cytometry.

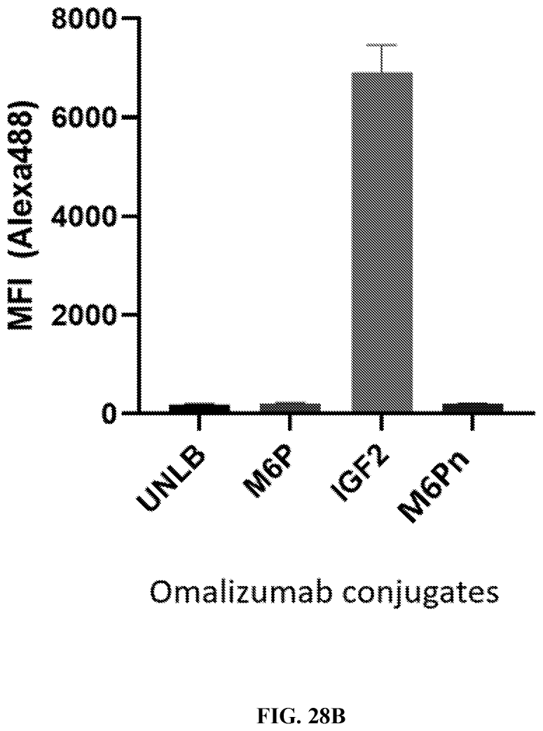

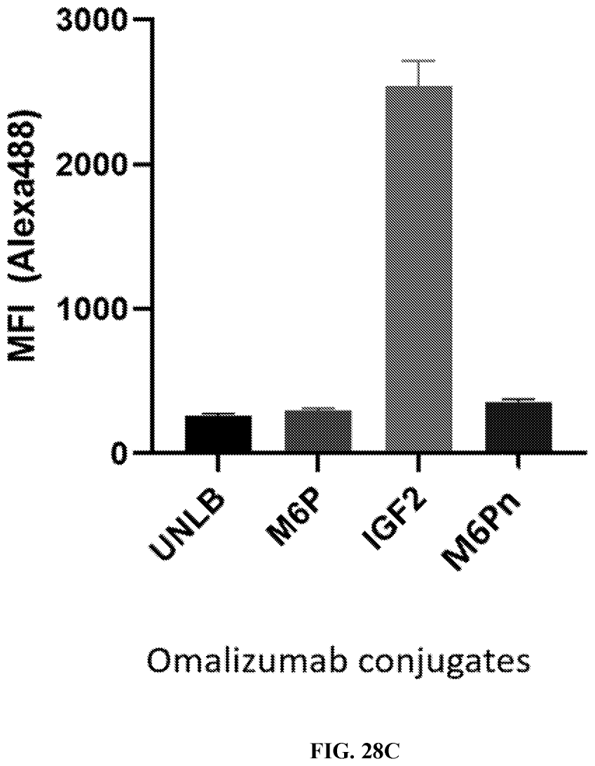

FIG. 28A shows a graph of MFI indicating extent of uptake for each composition in human Jurkat cells. FIG. 28B shows uptake in mouse C2C12 cells. FIG. 28C shows uptake in mouse fibroblasts. The cellular uptake is compared to omalizumab conjugates with glycan ligands for M6PR (mannose-6-phosphate ligand (M6P) or mannose-6-phosphonate analog (M6Pn, e.g., Compound I-7)) and unconjugated omalizumab (UNLB).

Exemplary bifunctional compound (1) IGF-2-omalizumab was internalized to similar degree as M6Pn-omalizumab in Jurkat cells. Internalization of compound (1) IGF-2-omalizumab was also observed in the mouse myocyte cell line C2C12 as well as primary mouse fibroblasts. No internalization of M6Pn-omalizumab or M6Pn-omalizumab conjugates was observed in either mouse cell type.

In Vitro Cell Uptake Assay with IR or IGF1R Receptor Inhibitors

The cell uptake assay is repeated in presence of IR and IGF1R blocking antibodies. Internalization of compound (1) IGF-2-omalizumab via IR and IGF1R cell surface receptors is reduced or eliminated in select cell types.

IGF2-Omalizumab is Internalized in K562^{M6PR-WT} Cells but not K562^{M6PR-KO} Cells

Omalizumab was conjugated to M6P, M6Pn, or IGF2 as described above, or left unconjugated (UNLB). The omalizumab compositions were then fluorescently labelled with Alexa 488 fluorescent dye as described above.

Uptake of the omalizumab-AF488 compositions was evaluated in human K562 M6PR-wild type (WT) cells or human K562 M6PR-knockout (KO) cells. Cells were incubated for 1 hour with composition and then analyzed by flow cytometry to measure mean fluorescent intensity (MFI) of cells.

FIG. 29 shows the results of a cell uptake assay that illustrate that exemplary bifunctional compound (1) IGF-2-omalizumab is internalized in wild type K562 cells having M6PR but not M6PR-knockout (KO) K562 cells. Similar results were observed for omalizumab conjugates with glycan ligands for M6PR (“M6P” or “M6Pn” (Compound I-7)). UNLB is the control unconjugated omalizumab.

No internalization of exemplary bifunctional compound (1) IGF-2-omalizumab was observed in K562^{M6PR-KO} cells suggesting uptake in this cell line is mediated via M6PR. K562 cells do not express the receptors 1R or IGF1R.

AAV8 Virus Particle Conjugates Bind to Neutralizing Antibody, ADK8

The AAV8-Compound I-7 conjugate described above, was tested for binding to ADK8, an AAV8 neutralizing antibody (NAb) that inhibits transduction efficiency of AAV8, including transduction efficiency of AAV8 into 2V6·11 cells and Jurkat cells. A shown in FIGS. 13A-13D, the presence of the ADK8 neutralizing antibody did not affect the transduction efficiency of the AAV8-Compound I-7 conjugate in 2V6·11 cells (FIGS. 13A and 13B) and Jurkat cells (FIGS. 13C and 13D), but, in contrast, substantially decreased the transduction efficiency of AAV8 alone. FIGS. 13A and 13C show the transduction efficiency as a percentage of GFP positive cells, whereas FIGS. 13B and 13D show mean fluorescence intensity (MFI). “Unlabeled” denotes AAV8 alone; “10 k” and “25 k” indicate the molar ratio of Compound I-7 to AAV8 (10 000:1 and 25 000:1, respectively).

Briefly, ADK8 Nab binding to GFP-AAV8 alone and to the AAV8-Compound I-7 conjugate were measured using an ELISA kit (Progen) according to manufacturer's instructions. The data shown in FIG. 14 indicate that the AAV8-Compound I-7 conjugate retains an ability to bind to ADK8 similar to that of GFP-AAV8 conjugate without Compound I-7. Binding to by the AAV8-Compound I-7 conjugate is a prerequisite for clearance of ADK8 by the Compound I-7 conjugate.

In performing these experiments. 2V6·11 cells (Elab Science) in DMEM (Gibco) with 10% fetal bovine serum (Gibco) or Jurkat cells (ATCC) in RPMI (Gibco) were seeded onto 96-well plates (Nunc) 24 h prior to infection. Unconjugated AAV8-GFP (Vigene) or conjugated AAV8-CMV-GFP at a multiplicity of infection (MOI) of 5×10⁴, were preincubated with 0, 2, 4, 8, or 16 ng of ADK8 antibody (Origene) for 30 min at 37° C. and then added to cells. After 72 hours, the medium was aspirated, cells were washed with PBS, and DMEM and FCS was added. After removing culture supernatant, the cells were washed with PBS and analyzed for GFP expression via flow cytometry on BioRad ZES Cell Analyzer (BioRad) and GFP expression analysis was performed.

Clearance of ADK8 by Anti-IgG2a Conjugate

AAV8 neutralizing antibody ADK8 (4 ng/mL) was incubated with increasing concentrations of Compound I-7-aIgG2a or aIgG2a alone for 30 minutes at room temperature. As an additional control, an isotype control antibody (mouse IgG2a) was incubated with increasing concentrations of Compound 1-7-αIgG. Complexes were then added to different densities of Jurkat cells (25 k, 50 k or 100 k/well) and incubated for 72 h. The supernatant was then removed and incubated with 15,000 AAV8-LUC particles for 30 min at 37° C. The complex was added to 2V6·11 cells and luciferase levels were evaluated 24h later using a luciferase assay kit (Promega) following manufacture's protocol and using a luminometer.

Data shown in FIG. 30 indicate that increasing concentrations of Compound I-7-aIgG2a conjugate can clear ADK8 antibody and increase in vitro AAV8 transduction. IC80 concentration, a 25-fold excess molar ratio of Compound I-7-aIgG2a is required to bring AAV8 transduction to isotype control level in 2V6·11 cells. Moreover, data shown in FIG. 30 also indicates that aIgG2a alone does not promote clearing of ADK8.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method of viral transduction, comprising:
contacting a cell with a modified viral composition to transduce the cell with the modified viral composition, wherein the modified viral composition comprises a bridging composition comprising a bridging moiety and a cell surface binding moiety, wherein the bridging moiety is capable of binding to a viral particle and the cell surface binding moiety is capable of binding to a cell surface receptor.

2. The method of claim 1, wherein the modified viral composition exhibits tropism for at least one cell type or tissue when compared to a viral composition comprising a viral particle alone.

3. The method of claim 2, wherein the at least one cell type or tissue is liver.

4. The method of claim 1, wherein the modified viral composition has increased ability to transduce at least one tissue as compared to a viral composition comprising a viral particle alone.

5. The method of claim 4, wherein the at least dine tissue is muscle tissue.

6. The method of claim 1, wherein the modified viral composition does not exhibit tropism for the cell.

7. The method of claim 1, wherein transduction efficiency of the viral particle into a cell is increased compared to transduction efficiency of a viral particle alone.

8. The method of claim 7, wherein the transduction efficiency is increased by 5%, 10%, 15%, 20%, 25% or 30% or more.

9. The method of claim 7, wherein the transduction efficiency is increased by at least 2-fold, 3-fold, 4-fold, 5-fold, 6-fold, 7-fold or greater.

10. The method of claim 1, wherein die viral particle of the modified viral composition is more potent than a viral particle alone.

11. The method of claim 1, wherein the cell is a virus transduction-resistant cell.

12. The method of claim 1, wherein the viral particle is an adenoviral (AV) particle, an adeno-associated viral (AAV) particle, or a lentiviral (LV) particle.

13. The method of claim 1 or 12, wherein the viral particle comprises a transgene.

14. The method of claim 13, wherein the viral particle is an AAV particle.

15. The method of claim 14, wherein the AAV particle comprises a polynucleotide comprising a transgene and at least one inverted terminal repeat (ITR).

16. The method of claim 15, wherein the polynucleotide comprises at least an ITR 5′ of the transgene (a “5′ ITR”) or an ITR 3′ of the transgene (a “3′ ITR”).

17. The method of claim 16, wherein the polynucleotide comprises a transgene flanked by a 5′ ITR and a 3′ ITR.

18. The method of claim 13 or 15, wherein the transgene is expressed in the transduced cell.

19. The method of any one of claim 13, 15, or 18, wherein the transgene encodes a polypeptide or RNA.

20. The method of claim 19, wherein the polypeptide is an AAT (alpha-1 anti-trypsin) polypeptide, an ADCC (aromatic L-amino acid decarboxylase) polypeptide, an antibody or an antigen-binding fragment of an antibody, a dystrophin polypeptide, a Factor VIII polypeptide, a Factor IX polypeptide, a GAA (acid alpha-glucosidase) polypeptide, a GAD (glutamate decarboxylase) polypeptide, a GDNF (glial cell line-derived neurotrophic factor) polypeptide, an ND4 (NADH dehydrogenase 4) polypeptide, a REP1 (Rab-escort protein 1) polypeptide, a REP65 (Retinal pigment epithelium-specific 65) polypeptide, a RPGR (retinitis pigmentosa GTPase regulator) polypeptide, a SERCA2a (sarcoplasmic reticulum calcium ATPase) polypeptide, an SMN (survival motor neuron) polypeptide, an anti-VEGF polypeptide, a VEGF-binding polypeptide, a TNFR (tumor necrosis factor receptor) polypeptide or a telomerase polypeptide.

21. The method of claim 18, wherein the transgene expression is achieved by administering a vector genome (vg) dose of the modified viral composition that is less than the dose that would be required of a viral composition comprising the viral particle alone.

22. The method of any one of the preceding claims, wherein the cell surface receptor is an internalizing receptor.

23. The method of claim 22, wherein the internalizing receptor is an endocytic receptor.

24. The method of any one of the preceding claims, wherein the cell surface receptor is mannose 6 phosphate receptor (M6PR), asialoglycoprotein receptor (ASGPR), or folate receptor.

25. The method of any of the preceding claims, wherein the cell surface binding moiety is a ligand capable of binding to an internalizing receptor.

26. The method of claim 25, wherein the ligand is insulin-like growth factor 2 (IFG2), galactose, N-acetylgalactosamine (GalNAc), folate, folic acid, mannose-6-phosphate (M6P), mannose-6-phosphonate (M6Pn), or derivatives thereof.

27. The method of any of the preceding claims, wherein the viral particle is directly attached to the Heterologous cell surface binding moiety.

28. The method of any of claims 1-26, wherein the viral particle is attached to the heterologous cell surface binding moiety via a linker.

29. The method of any of the preceding claims, wherein the transduced cell is a mammalian cell.

30. The method of claim 29, wherein the transduced cell is a muscle cell, neural cell, liver cell, cardiac cell, lung cell, immune cell, or kidney cell.

31. The method of any of the preceding claims, wherein the transduced cell is an AAV transduction-resistant cell.

32. The method of any of the preceding claims, wherein the contacting occurs in the presence of neutralizing antibodies.

33. A method of viral transduction, comprising:
administering to a subject a pharmaceutical composition comprising a modified viral composition, wherein the modified viral composition comprises a bridging composition comprising a bridging moiety and a cell surface binding moiety, wherein the bridging moiety is capable of binding to a viral particle, the cell surface binding moiety is capable of binding to a cell surface receptor, and the modified viral composition enters a target cell in the subject.

34. A method of viral transduction, comprising:
contacting a target cell from a subject ex vivo with a modified viral composition to generate a transduced cell, wherein the modified viral composition comprises a bridging composition comprising a bridging moiety and a cell surface binding moiety, wherein the bridging moiety is capable of binding to a viral particle and the cell surface binding moiety is capable of binding to a cell surface receptor; and
administering the transduced cell to the subject.

35. The method of claim 33 or 34, wherein the modified viral composition exhibits tropism for at least one cell type or tissue when compared to a viral composition comprising a viral particle alone.

36. The method of claim 35, wherein the at least one cell type or tissue is liver.

37. The method of claim 33 or 34, wherein the modified viral composition has increased ability to transduce at least one tissue as compared to a viral composition comprising a viral particle alone.

38. The method of claim 37, wherein the at least one tissue is muscle tissue.

39. The method of claim 33 or 34, wherein transduction efficiency of the viral particle into a cell is increased compared to transduction efficiency of a viral particle alone.

40. The method of claim 39, wherein the transduction efficiency is increased by 5%, 10%, 15%, 20%, 25% or 30% or more.

41. The method of claim 39, wherein the transduction efficiency is increased by at least 2-fold, 3-fold, 4-fold, 5-fold, 6-fold, 7-fold or greater.

42. The method of claim 33 or 34, wherein the viral particle of the modified viral composition is more potent than a viral particle alone.

43. The method of claim 33 or 34, wherein the target cell is a virus transduction-resistant cell.

44. The method of claim 33 or 34, wherein the viral particle is an adenoviral (AV) particle, an adeno-associated viral (AAV) particle, or a lentiviral (LV) particle.

45. The method of any one of claim 33, 34, or 44, wherein the viral particle comprises a transgene.

46. The method of claim 45, wherein the viral particle is an AAV particle.

47. The method of claim 46, wherein the AAV particle comprises a polynucleotide comprising a transgene and at least one inverted terminal repeat (ITR).

48. The method of claim 47, wherein the polynucleotide comprises at least an ITR 5′ of the transgene (a “5′ ITR”) or an ITR 3′ of the transgene (a “3′ ITR”).

49. The method of claim 48, wherein the polynucleotide comprises a transgene flanked by a 5′ ITR and a 3′ ITR.

50. The method of claim 45 or 47, wherein the transgene is expressed in the transduced cell.

51. The method of any one of claim 45, 47, or 50, wherein the transgene encodes a polypeptide or RNA.

52. The method of claim 51, wherein the polypeptide is an AAT (alpha-1 anti-trypsin) polypeptide, an ADCC (aromatic L-amino acid decarboxylase) polypeptide, an antibody or an antigen-binding fragment of an antibody, a dystrophin polypeptide, a Factor VIII polypeptide, a Factor IX polypeptide, a GAA (acid alpha-glucosidase) polypeptide, a GAD (glutamate decarboxylase) polypeptide, a GDNF (glial cell line-derived neurotrophic factor) polypeptide, an ND4 (NADH dehydrogenase 4) polypeptide, a REP1 (Rab-escort protein 1) polypeptide, a REP65 (Retinal pigment epithelium-specific 65) polypeptide, a RPGR (retinitis pigmentosa GTPase regulator) polypeptide, a SERCA2a (sarcoplasmic reticulum calcium ATPase) polypeptide, an SAM (survival motor neuron) polypeptide, anti-VEGF polypeptide, a VEGF-binding polypeptide, a TNFR (tumor necrosis factor receptor) polypeptide or a telomerase polypeptide.

53. The method of claim 50, wherein the transgene expression is achieved by administering a vector genome (vg) dose of the modified viral composition that is less than the dose that would be required of a viral composition comprising the viral particle alone.

54. The method of any one of claims 33-53, wherein the cell surface receptor is an internalizing receptor.

55. The method of claim 54, wherein the internalizing receptor is an endocytic receptor.

56. The method of any one of claims 33-55, wherein the cell surface receptor is mannose 6 phosphate receptor (M6PR), asialoglycoprotein receptor (ASGPR), or folate receptor.

57. The method of any one of claims 33-56, wherein the cell surface binding moiety is a ligand capable of binding to an internalizing receptor.

58. The method of claim 57, wherein the ligand is insulin-like growth factor 2 (IFG2), galactose, N-acetylgalactosamine (GalNAc), folate, folic acid, mannose-6-phosphate (M6P), mannose-6-phosphonate (M6Pn), or derivatives thereof.

59. The method of any one of claims 33-58, wherein the viral particle is directly attached to the heterologous cell surface binding moiety.

60. The method of any of claims 33-58, wherein the viral particle is attached to the heterologous surface binding moiety via a linker.

61. The method of any one of claims 33-60, wherein the target cell is a mammalian cell.

62. The method of claim 61, wherein the target cell is a muscle cell, neural cell, liver cell, cardiac cell, lung cell, immune cell, or kidney cell.

63. The method of any one of claims 33-62, wherein the target cell is an AAV transduction-resistant cell.

64. The method of any one of claims 33-63, wherein the administering occurs in the presence of neutralizing antibodies

65. A pharmaceutical composition comprising a modified viral composition, wherein the modified viral composition comprises a bridging composition comprising a bridging moiety and a cell surface binding moiety, wherein the bridging moiety is attached to a viral particle and the cell surface binding moiety, and a pharmaceutically acceptable carrier.

66. The pharmaceutical composition of claim 65, wherein the viral particle comprises a transgene.

67. The pharmaceutical composition of claim 66, wherein the transgene encodes a therapeutic polypeptide.

68. The pharmaceutical composition of claim 67, wherein the therapeutic polypeptide is an enzyme.

69. The pharmaceutical composition of claim 68, wherein the enzyme is acid alpha-glucosidase (GAA), phenylalanine ammonia-lyase, alpha-galactosidase A, glucocerebrosidase (GCase) aspartylglucosaminidase (AGA), alpha-L-iduronidase, iduronate sulfatase, sulfaminase, alpha-N-acetylgtucosaminidase (NAGLU), alpha-glucosaminide N-acetyltransferase (1-1GSNAT), N-acetylglucosamine 6-sulfatase (GNS), N-glucosamine 3-O-sulfatase (ARSG), N-acetylgalactosamine 6-sulfatase, beta-glucuronidase, palmitoyl protein tioesterase (PPT1), tripeptidyl peptidase (TPP1), acid sphingomyelinase, or lysosomal acid lipase.

70. A method of treatment, comprising administering an effective amount of the pharmaceutical composition of any one of claims 65-69 to a subject in need thereof.

71. The method of claim 70, wherein the subject has previously been administered a viral composition.

72. The method of claim 70 or 71, wherein the method generates cells transduced with the viral composition in the subject.

73. The method of claim 70 or 72, wherein the effective amount of the pharmaceutical composition administered is less than the effective amount of a pharmaceutical composition comprising an unmodified viral particle that comprises the transgene.

74. A bridging composition, comprising a bridging moiety and a cell surface binding moiety, wherein the bridging moiety is capable of binding to a viral composition and the cell surface binding moiety is capable of binding to a cell surface receptor.

75. The bridging composition of claim 74, wherein the bridging moiety binds to a viral particle, virus capsid, virus envelope, or viral protein.

76. The bridging composition of claim 75, wherein the bridging moiety binds to a viral particle comprising a transgene.

77. The bridging composition of any one of claims 74 to 76, wherein the bridging moiety binds an adenovirus adeno-associated virus (AAV), retrovirus, or herpes simplex viral composition.

78. The bridging composition of claim 77, wherein the bridging moiety binds an adeno-associated virus (AAV) composition.

79. The bridging composition of claim 78, wherein the bridging moiety binds an AAV composition of AAV serotype AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV 11, AAV12, AAV13, AAV LK03, AAVrh74, AAV DJ, AAV Anc81, Anc82, Anc83, Anc84, Anc110, Anc113, Anc126, Or Anc127, AAV hu·37, AAV_go·1, AAV LK03, or AAV rh74

80. The bridging composition of any one of claims 74 to 79, wherein the cell surface receptor is an internalizing receptor.

81. The bridging composition of claim 80, wherein the internalizing receptor is an endocytic receptor.

82. The bridging composition of any one of claims 74 to 80, wherein the cell surface receptor is a mannose-6-phosphate receptor (M6PR), an asialoglycoprotein receptor (ASGPR), or a folate receptor.

83. The bridging composition of claim 82, wherein the M6PR is a cation-independent M6P receptor (CI-M6PR).

84. The bridging composition of any one of claims 74 to 83, wherein the cell surface binding moiety is a ligand capable of binding to an internalizing receptor.

85. The bridging composition of any one of claims 74 to 84, wherein the heterologous cell surface binding moiety comprises a sugar moiety.

86. The bridging composition of claim 85, wherein the sugar moiety is mannose-6-phosphate (M6P), mannose-6-phosphonate (M6Pn), or a variant thereof.

87. The bridging composition of any one of claims 74 to 84, wherein the cell surface binding moiety comprises a protein.

88. The bridging composition of claim 87, wherein the protein is insulin-like growth factor 2 (IGF2).

89. The bridging composition of any one of claims 74 to 88, wherein the bridging moiety comprises a protein.

90. The bridging composition of claim 89, wherein the protein is an antibody or an antigen-binding fragment of an antibody.

91. The bridging composition of claim 90, wherein the antibody is an IgG antibody.

92. The bridging composition of claim 91, wherein the antibody is an IgG1 antibody.

93. The bridging composition of claim 91, wherein the antibody is an IgG2 antibody.

94. The bridging composition of any one of claims 90 to 93, wherein the antibody is a humanized antibody.

95. The bridging composition of any one of claims 90 to 93, wherein the antibody is a human antibody.

96. The bridging composition of any one of claims 90 to 93, wherein the antibody is a Fab.

97. The bridging composition of any one of claims 90 to 93, wherein the antibody or antigen-binding fragment of an antibody is a single-chain antibody or single-chain antigen binding fragment thereof.

98. The bridging composition of any one of claims 90 to 97, wherein the antibody or antigen-binding fragment is capable of binding to an AAV composition of more than one AAV serotype.

99. The bridging composition of any one of claims 90 to 98, wherein the bridging moiety and the cell surface binding moiety are conjugated to each other.

100. The bridging composition of claim 99, wherein the bridging moiety and the cell surface binding moiety are conjugated to each other via a linker.

101. The bridging composition of any one of claims 74 to 100, wherein the bridging moiety comprises a protein and the cell surface binding moiety comprises a protein, and the proteins are fused to each other.

102. The bridging composition of claim 101, wherein the bridging moiety protein and the cell surface binding moiety protein are fused to each other via an intervening amino acid sequence.

103. The bridging composition of any one of claims 74 to 102, wherein the bridging composition is of formula (I): 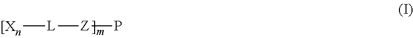
or a pharmaceutically acceptable salt thereof,
wherein:
X is the cell surface binding moiety capable of binding to a cell surface receptor;
L is an optional linker;
Z is a residual moiety resulting from the attachment of Xₙ (or L, if present) to P; and
P is a bridging moiety that is capable of binding to a viral composition (e.g., a viral particle, viral capsid, a viral envelope or a viral protein).

104. A modified viral composition, comprising:
a viral composition; and
a bridging composition according to any one of claims 74 to 103 comprising a bridging moiety and a cell surface binding moiety, wherein the bridging moiety is capable of binding to the viral composition (e.g., viral particle) and the cell surface binding moiety is capable of binding to a cell surface receptor.

105. The modified viral composition of claim 104, wherein the viral composition is an empty virus particle.

106. A pharmaceutical composition comprising the modified viral composition of claim 104 or 105, and a pharmaceutically acceptable carrier.

107. A method of reducing neutralizing antibody (Nab) titer in a subject in need thereof, comprising administering a modified viral composition of claim 105, or the pharmaceutical composition of claim 106 to the subject, such that NAb titer in the subject is reduced.

108. The method of claim 107, wherein the modified viral composition comprises an empty virus particle.

109. The method of claim 107, wherein the modified viral composition comprises a viral protein.

110. The method of any one of claims 107 to 109, wherein the subject is a human in need of viral therapy, and wherein administering the modified viral composition is performed prior to the onset of the viral therapy.

111. The method of claim 110, wherein administering the modified viral composition is performed 1 to 6 hours prior to the onset of the viral therapy.

112. The method of claim 110 or 111, further comprising administering the viral therapy to the subject following the administering of the modified viral composition.

113. The method of any one of claims 107 to 112, wherein the subject is a human undergoing viral therapy.

114. The method of claim 113, wherein the modified viral composition is administered to the subject concurrently with the viral therapy.

115. The method of any one of claims 107 to 114, wherein the subject is a human who has previously undergone viral therapy and is in need of additional viral therapy.

116. The method of claim 115, wherein administering the modified viral composition is performed 1 to 6 hours prior to onset of the additional viral therapy.

117. The method of claim 115 or 116, further comprising administering the additional viral therapy to the subject following administering the modified viral composition.

118. A method of reducing AAV neutralizing antibody (Nab) titer in a subject in need thereof, comprising: administering a modified viral composition of claim 105 or the pharmaceutical composition of claim 106 to the subject, wherein the viral composition comprises an AAV composition, such that NAb titer in the subject is reduced.

119. The method of claim 118, wherein the modified viral composition comprises an empty AAV particle.

120. The method of claim 119, wherein the modified viral composition comprises an AAV viral protein.

121. The method of claim 120, wherein the AAV viral protein is an AAV VP1, VP2 or VP3 protein.

122. The method of any one of claims 120 to 121, wherein the subject is a human in need of AAV-based gene therapy, and wherein administering the modified viral composition comprising the AAV composition is performed prior to the onset of the gene therapy.

123. The method of claim 122, wherein administering the modified viral composition comprising the AAV composition is performed 1 to 6 hours prior to the onset of the gene therapy.

124. The method of claim 122 or 123, further comprising administering the gene therapy to the subject following the administering of the modified viral composition.

125. The method of any one of claims 118 to 124, wherein the subject is a human undergoing AAV-based gene therapy.

126. The method of claim 125, wherein the modified viral composition comprising the AAV composition is administered to the subject concurrently with the gene therapy.

127. The method of any one of claims 118 to 126, wherein the subject is a human who has previously undergone gene therapy and is in need of additional AAV-based gene therapy.

128. The method of claim 127, wherein administering the modified viral composition comprising the AAV composition is performed 1 to 6 hours prior to onset of the additional gene therapy.

129. The method of claim 126 or 127, further comprising administering the additional gene therapy to the subject following administering the modified viral composition.

130. The method of any one of claims 118 to 129, wherein the AAV of the AAV composition is an AAV1, AAV2, AAV2i8, AAV3, AAV3-B, AAV4, AAV5, AAV6, AAV7, AAV8, AAVrh8, AAVrh8R, or AAV rh·8, AAV9, AAV10, AAVrh10, AAV11, AAV12, AAV13, AAV LK03, AAVrh74, AAV DJ, AAV Anc81, Anc82, Anc83, Anc84, Anc110, Anc113, Anc126, or Anc127, AAV hu·37, AAV rh·8, AAV_go·1, AAV LK03, or AAV rh74 serotype.


SEQUENCE LISTING (98 sequences)

SEQ ID NO: 1 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 2 - protein, 62 aa, Artificial Sequence
  Description of Artificial Sequence Synthetic polypeptide
AALCGGELVDTLQFVCGDRGFYFSRPASRVSRRSRGIVEECCFRSCDLALLETYCATPAKSE

SEQ ID NO: 3 - protein, 63 aa, Artificial Sequence
  Description of Artificial Sequence Synthetic polypeptide
SRTLCGGELVDTLQPVCGDRGFLFSRPASRVSRRSRGIVEECCFRSCDLALLETYCATPARSE

SEQ ID NO: 4 - protein, 61 aa, Homo sapiens
TLCGGELVDTLQFVCGDRGFYFSRPASRVSRRSRGIVEECCFRSCDLALLETYCATPAKSE

SEQ ID NO: 5 - protein, 60 aa, Homo sapiens
LCGGELVDTLQFVCGDRGFYFSRPASRVSRRSRGIVEECCFRSCDLALLETYCATPAKSE

SEQ ID NO: 6 - protein, 60 aa, Artificial Sequence
  Description of Artificial Sequence Synthetic polypeptide
LCGGELVDTLQFVCGDRGFYFSRPASRVSARSRGIVEECCFRSCDLALLETYCATPAKSE

SEQ ID NO: 7 - protein, 10 aa, Artificial Sequence
  Description of Artificial Sequence Synthetic peptide
ELVDTLQFVS

SEQ ID NO: 8 - protein, 10 aa, Artificial Sequence
  Description of Artificial Sequence Synthetic peptide
ELVDWLQFVS

SEQ ID NO: 9 - protein, 10 aa, Artificial Sequence
  Description of Artificial Sequence Synthetic peptide
ELVDYLQFVS

SEQ ID NO: 10 - protein, 10 aa, Artificial Sequence
  Description of Artificial Sequence Synthetic peptide
ELVDTLQFVW

SEQ ID NO: 11 - protein, 10 aa, Artificial Sequence
  Description of Artificial Sequence Synthetic peptide
ELVDFLQFVS

SEQ ID NO: 12 - protein, 10 aa, Artificial Sequence
  Description of Artificial Sequence Synthetic peptide
ELVDTLQFVR

SEQ ID NO: 13 - protein, 12 aa, Unknown
  Description of Unknown P431 peptide sequence
VHWDFRQWWQPS

SEQ ID NO: 14 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 15 - protein, 183 aa, Homo sapiens
MGIPMGKSMLVLLTFLAFASCCIAAYRPSETLCGGELVDTLQFVCGDRGFYFRLPGRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 16 - protein, 236 aa, Homo sapiens
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 17 - protein, 5 aa, Artificial Sequence
  Description of Artificial Sequence Synthetic peptide
GGGGA

SEQ ID NO: 18 - protein, 5 aa, Artificial Sequence
  Description of Artificial Sequence Synthetic peptide
GGGGS

SEQ ID NO: 19 - protein, 219 aa, Artificial Sequence
  Description of Artificial Sequence Synthetic polypeptide
DVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 284 aa, Artificial Sequence
  Description of Artificial Sequence Synthetic polypeptide
DVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSLCGGELVDTLQFVCGDRGFYFSRPASRVSRRSRGIVEECCFRSCDLALLETYCATPAKSE

SEQ ID NO: 21 - protein, 284 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 219 aa, Artificial Sequence
  Description of Artificial Sequence Synthetic polypeptide
DVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 284 aa, Artificial Sequence
  Description of Artificial Sequence Synthetic polypeptide
DVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLCGGELVDTLQFVCGDRGFYFSRPASRVSRRSRGIVEECCFRSCDLALLETYCATPAKSE

SEQ ID NO: 24 - protein, 284 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDVVMTQTPLTLSVTIGQPASISCKSSQSLLESDGKTYLNWLLQRPGQSPKRLIYLVSTLDSGVPDRFTGSGSGTDFTLKISRLEAEDLGVYYCWQGTHFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 451 aa, Artificial Sequence
  Description of Artificial Sequence Synthetic polypeptide
DVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 451 aa, Artificial Sequence
  Description of Artificial Sequence Synthetic polypeptide
DVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 516 aa, Artificial Sequence
  Description of Artificial Sequence Synthetic polypeptide
DVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSLCGGELVDTLQFVCGDRGFYFSRPASRVSRRSRGIVEECCFRSCDLALLETYCATPAKSE

SEQ ID NO: 28 - protein, 516 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 29 - protein, 516 aa, Artificial Sequence
  Description of Artificial Sequence Synthetic polypeptide
DVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLCGGELVDTLQFVCGDRGFYFSRPASRVSRRSRGIVEECCFRSCDLALLETYCATPAKSE

SEQ ID NO: 30 - protein, 516 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDVKLVESGGGLVKPGGSLKLSCADSGFSFSSYSMSWVRQTPEKRLEWVATITSGGDYTYYPDSVKGRFTISKDNARNTLYLQMSSLRSEDTAMYYCIRDFYGSTYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Artificial Sequence
  Description of Artificial Sequence Synthetic polypeptide
DIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 279 aa, Artificial Sequence
  Description of Artificial Sequence Synthetic polypeptide
DIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSLCGGELVDTLQFVCGDRGFYFSRPASRVSRRSRGIVEECCFRSCDLALLETYCATPAKSE

SEQ ID NO: 33 - protein, 279 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 34 - protein, 214 aa, Artificial Sequence
  Description of Artificial Sequence Synthetic polypeptide
DIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 279 aa, Artificial Sequence
  Description of Artificial Sequence Synthetic polypeptide
DIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLCGGELVDTLQFVCGDRGFYFSRPASRVSRRSRGIVEECCFRSCDLALLETYCATPAKSE

SEQ ID NO: 36 - protein, 279 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 450 aa, Artificial Sequence
  Description of Artificial Sequence Synthetic polypeptide
SDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 38 - protein, 450 aa, Artificial Sequence
  Description of Artificial Sequence Synthetic polypeptide
SDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 515 aa, Artificial Sequence
  Description of Artificial Sequence Synthetic polypeptide
SDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSLCGGELVDTLQFVCGDRGFYFSRPASRVSRRSRGIVEECCFRSCDLALLETYCATPAKSE

SEQ ID NO: 40 - protein, 515 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSSDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 41 - protein, 515 aa, Artificial Sequence
  Description of Artificial Sequence Synthetic polypeptide
SDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLCGGELVDTLQFVCGDRGFYFSRPASRVSRRSRGIVEECCFRSCDLALLETYCATPAKSE

SEQ ID NO: 42 - protein, 515 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSSDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 219 aa, Artificial Sequence
  Description of Artificial Sequence Synthetic polypeptide
DIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 44 - protein, 284 aa, Artificial Sequence
  Description of Artificial Sequence Synthetic polypeptide
DIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSLCGGELVDTLQFVCGDRGFYFSRPASRVSRRSRGIVEECCFRSCDLALLETYCATPAKSE

SEQ ID NO: 45 - protein, 219 aa, Artificial Sequence
  Description of Artificial Sequence Synthetic polypeptide
DIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 284 aa, Artificial Sequence
  Description of Artificial Sequence Synthetic polypeptide
DIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLCGGELVDTLQFVCGDRGFYFSRPASRVSRRSRGIVEECCFRSCDLALLETYCATPAKSE

SEQ ID NO: 47 - protein, 284 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 442 aa, Artificial Sequence
  Description of Artificial Sequence Synthetic polypeptide
QVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 49 - protein, 442 aa, Artificial Sequence
  Description of Artificial Sequence Synthetic polypeptide
QVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 507 aa, Artificial Sequence
  Description of Artificial Sequence Synthetic polypeptide
QVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSLCGGELVDTLQFVCGDRGFYFSRPASRVSRRSRGIVEECCFRSCDLALLETYCATPAKSE

SEQ ID NO: 51 - protein, 507 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSQVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 507 aa, Artificial Sequence
  Description of Artificial Sequence Synthetic polypeptide
QVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLCGGELVDTLQFVCGDRGFYFSRPASRVSRRSRGIVEECCFRSCDLALLETYCATPAKSE

SEQ ID NO: 53 - protein, 507 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSQVQLQQSAAELVRPGASVTLSCKASGYTFNDHEMHWVKQTPVYGLEWIGAIDPETGGTAYNQKFKGKATLTADKSSSTAYMELRSLTSEDSAVYYCTGEGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 55 - protein, 735 aa, Adeno-associated virus 2 [sequence omitted]

SEQ ID NO: 56 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 57 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 58 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 59 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 61 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 62 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 742 aa, Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 64 - protein, 736 aa, Non-human primate Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 65 - protein, 738 aa, Non-human primate Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 66 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGVGSNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTGTAGTQTLQFSQAGPSSMANQARNWVPGPCYRQQRVSTTTNQNNNSNFAWTGATKYHLNGRDSLMNPGVAMASHKDDEDRFFPSSGVLIFGKQGAGNDNVDYSQVMITNEEEIKTTNPVATEEYGAVATNNQSANTQAQTGLVHNQGVLPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 67 - protein, 736 aa, Recombinant adeno-associated virus vector AAV-LK19
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 68 - protein, 284 aa, Artificial Sequence
  Description of Artificial Sequence Synthetic polypeptide
LCGGELVDTLQFVCGDRGFYFSRPASRVSRRSRGIVEECCFRSCDLALLETYCATPAKSEGGGGSDIVMSQSPSSLTVSVGEKVTMSCKSSQSLLYSTNQKNYLAWYQQKPGQSPKLLIFWASTRESGVPDRFTGSGSGTDFTLTISSVKAEDLAVYYCQQYYRYLTFGTGTKLELRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 69 - protein, 5 aa, Artificial Sequence
  Description of Artificial Sequence Synthetic peptide
  MOD_RES (3)..(3): Any amino acid
LPXTG

SEQ ID NO: 70 - protein, 5 aa, Artificial Sequence
  Description of Artificial Sequence Synthetic peptide
LLQGA

SEQ ID NO: 71 - protein, 599 aa, Adeno-associated virus 1
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 72 - protein, 534 aa, Adeno-associated virus 1
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 73 - protein, 598 aa, Adeno-associated virus 2
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 74 - protein, 533 aa, Adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 75 - protein, 599 aa, Adeno-associated virus 3
TAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 76 - protein, 534 aa, Adeno-associated virus 3
MASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 77 - protein, 598 aa, Adeno-associated virus 4
TAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 78 - protein, 538 aa, Adeno-associated virus 4
MRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 79 - protein, 588 aa, Adeno-associated virus 5
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 80 - protein, 532 aa, Adeno-associated virus 5
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 81 - protein, 599 aa, Adeno-associated virus 6
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 82 - protein, 534 aa, Adeno-associated virus 6
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 83 - protein, 600 aa, Adeno-associated virus 7
TAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 84 - protein, 534 aa, Adeno-associated virus 7
VAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 85 - protein, 601 aa, Adeno-associated virus 8
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 86 - protein, 535 aa, Adeno-associated virus 8
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 87 - protein, 599 aa, Adeno-associated virus 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - protein, 534 aa, Adeno-associated virus 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 605 aa, Adeno-associated virus 12
TAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 90 - protein, 537 aa, Adeno-associated virus 12
MRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 91 - protein, 599 aa, Non-human primate Adeno-associated virus
TAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 92 - protein, 534 aa, Non-human primate Adeno-associated virus
MASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 93 - protein, 601 aa, Non-human primate Adeno-associated virus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 94 - protein, 535 aa, Non-human primate Adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 95 - protein, 599 aa, Adeno-associated virus
TAPGKKRPVEQSPQEPDSSSGIGKTGQQPAKKRLNFGQTGDSESVPDPQPLGEPPAAPSGVGSNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTGTAGTQTLQFSQAGPSSMANQARNWVPGPCYRQQRVSTTTNQNNNSNFAWTGATKYHLNGRDSLMNPGVAMASHKDDEDRFFPSSGVLIFGKQGAGNDNVDYSQVMITNEEEIKTTNPVATEEYGAVATNNQSANTQAQTGLVHNQGVLPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 96 - protein, 534 aa, Adeno-associated virus
MASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTGTAGTQTLQFSQAGPSSMANQARNWVPGPCYRQQRVSTTTNQNNNSNFAWTGATKYHLNGRDSLMNPGVAMASHKDDEDRFFPSSGVLIFGKQGAGNDNVDYSQVMITNEEEIKTTNPVATEEYGAVATNNQSANTQAQTGLVHNQGVLPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 97 - protein, 599 aa, Recombinant adeno-associated virus vector AAV-LK19
TAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 98 - protein, 534 aa, Recombinant adeno-associated virus vector AAV-LK19
MASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL